(19) United States
(12) Patent Application Publication
(10) Pub. No.: US 2019/0367612 A1
(43) Pub. Date: Dec. 5, 2019

(54) ANTI-GPRC5D ANTIBODY AND MOLECULE CONTAINING SAME

(71) Applicant: Daiichi Sankyo Company, Limited, Tokyo (JP)

(72) Inventors: Takashi CHAEN, Tokyo (JP); Toshiaki OHTSUKA, Tokyo (JP); Kenji IIDA, Tokyo (JP); Kensuke NAKAMURA, Tokyo (JP); Takahide ABURATANI, Tokyo (JP); Junya ICHIKAWA, Tokyo (JP); Shota KUDO, Tokyo (JP); Chayne Lee PISCITELLI, Vancouver (CA); Mario SANCHES, Vancouver (CA)

(73) Assignee: Daiichi Sankyo Company, Limited, Tokyo (JP)

(21) Appl. No.: 16/483,211

(22) PCT Filed: Jun. 2, 2018
(86) PCT No.: PCT/JP2018/003888

§ 371 Date: Aug. 2, 2019

(30) Foreign Application Priority Data
Feb. 7, 2017 (JP) 2017-020220

(51) Int. Cl. C07K 16/28; C07K 16/46; A61P 35/00

(52) U.S. Cl. CPC C07K 16/2809; C07K 16/46; A61P 35/00; C07K 2317/565; C07K 2317/31; C07K 2317/622; C07K 2317/54; C07K 2317/55; C07K 2317/569; C07K 2317/24

(57) ABSTRACT

The present disclosure provides a novel antibody that binds to human GPRC5D; a molecule with human GPRC5D binding activity, which comprises the antibody; and an anti-tumor pharmaceutical composition comprising the antibody or the molecule as an active.

### TECHNICAL FIELD

The present invention relates to a novel anti-GPRC5D antibody and a molecule comprising the antibody.

### BACKGROUND ART

G-protein coupled receptor family C group 5 member D (GPRC5D) is one of the G-protein coupled receptors found by the homology search of the EST database using the amino acid sequences of a series of human GPCRs (Non Patent Literature 1). GPCR family C group 5 receptors (GPRC5 receptors) have 4 subtypes (GPRC5A, GPRC5B, GPRC5C, and GPRC5D) and are also known as retinoic acid-inducible orphan G protein-coupled receptors (RAIG) because their expression is induced by retinoic acid stimulation (Non Patent Literature 2). However, the biological functions or biological ligand of GPRC5D, the subtype of G protein to be coupled therewith, etc. have not yet been revealed.

As for the association of the GPRC5D gene with cancers, GPRC5D is known to be highly expressed in multiple myeloma. Specifically, it is known that, for example: the overexpression of GPRC5D correlates with the poor prognosis of multiple myeloma patients (Non Patent Literature 3); and the proportion of cells expressing GPRC5D is decreased by the medication of multiple myeloma patients (Non Patent Literature 4). Such association of the overexpression of GPRC5D with cancers suggests the possibility that GPRC5D serves as an excellent therapeutic target for cancers. Furthermore, there is also a report on an anti-GPRC5D antibody and a bispecific antibody that comprises the antibody and an anti-CD3 antibody and exhibits binding activity against 3T3 cells expressing GPRC5D (Patent Literature 1) exogenously. However, medical pharmaceutical products targeting GPRC5D have not yet been developed.

### CITATION LIST

### Patent Literature

[Patent Literature 1] International Publication No. WO2016/090329

### Non Patent Literature

[Non Patent Literature 1] H Brauner-Osborne, et al., Biochim Biophys Acta., published in April 2001, Vol. 1518 (No. 3), p. 237-248
[Non Patent Literature 2] S Inoue, et al., Journal of Investigative Dermatology, published in March 2004, Vol. 122 (No. 3), p. 565-573
[Non Patent Literature 3] J Atamaniuk, et al., European Journal of Clinical Investigation, published in May 2012, Vol. 42 (No. 9), p. 953-960
[Non Patent Literature 4] Y Cohen, et al., Hematology), published in November 2013, Vol. 18 (No. 6), p. 348-351

### SUMMARY OF INVENTION

### Technical Problem

An object of the present invention is to provide an anti-GPRC5D antibody having an anticancer effect, an antigen-binding fragment of the antibody, and a molecule comprising the antibody or antigen-binding fragment of the antibody.

Another object of the present invention is to provide a pharmaceutical composition comprising the antibody or antigen-binding fragment of the antibody, or the molecule, etc.

An alternative object of the present invention is to provide a polynucleotide comprising a nucleotide sequence encoding the amino acid sequence of the antibody or antigen-binding fragment of the antibody, or the molecule, a vector having an insert of the polynucleotide, a cell transfected with the polynucleotide or vector, and a method for producing the antibody or antigen-binding fragment of the antibody, or the molecule, comprising the step of culturing the cell. A further alternative object of the present invention is to provide a method for treating a cancer using the antibody or antigen-binding fragment of the antibody, or the molecule.

### Solution to Problem

The present inventors have conducted diligent studies to attain the objects and have completed the present invention by developing a novel anti-GPRC5D antibody and finding that the antibody has an anticancer effect.

The present invention provides:
(1) An antibody or an antigen-binding fragment of the antibody, wherein the antibody comprises a heavy chain variable region and a light chain variable region described in any one of the following (I), (II), or (III):
(II)
a heavy chain variable region comprising
heavy chain CDR1 consisting of the amino acid sequence represented by SEQ ID NO: 48,
heavy chain CDR2 consisting of the amino acid sequence represented by SEQ ID NO: 49, and
heavy chain CDR3 consisting of the amino acid sequence represented by SEQ ID NO: 50,
and
a light chain variable region comprising
light chain CDR1 consisting of the amino acid sequence represented by SEQ ID NO: 57,
light chain CDR2 consisting of the amino acid sequence represented by SEQ ID NO: 58, and
light chain CDR3 consisting of the amino acid sequence represented by SEQ ID NO: 59,
(I)
a heavy chain variable region comprising
heavy chain CDR1 consisting of the amino acid sequence represented by SEQ ID NO: 45,
heavy chain CDR2 consisting of the amino acid sequence represented by SEQ ID NO: 46, and
heavy chain CDR3 consisting of the amino acid sequence represented by SEQ ID NO: 47,
and
a light chain variable region comprising
light chain CDR1 consisting of the amino acid sequence represented by SEQ ID NO: 54,
light chain CDR2 consisting of the amino acid sequence represented by SEQ ID NO: 55, and
light chain CDR3 consisting of the amino acid sequence represented by SEQ ID NO: 56,
and
(III)
a heavy chain variable region comprising
heavy chain CDR1 consisting of the amino acid sequence represented by SEQ ID NO: 51,
heavy chain CDR2 consisting of the amino acid sequence represented by SEQ ID NO: 52, and
heavy chain CDR3 consisting of the amino acid sequence represented by SEQ ID NO: 53,
and
a light chain variable region comprising
light chain CDR1 consisting of the amino acid sequence represented by SEQ ID NO: 60,
light chain CDR2 consisting of the amino acid sequence represented by SEQ ID NO: 61, and
light chain CDR3 consisting of the amino acid sequence represented by SEQ ID NO: 62
and binds to human GPRC5D;
(2) The antibody or antigen-binding fragment of the antibody according to (1), wherein the heavy chain variable region and the light chain variable region are the heavy chain variable region and the light chain variable region described in (II);
(3) The antibody or antigen-binding fragment of the antibody according to (1), wherein the heavy chain variable region and the light chain variable region are the heavy chain variable region and the light chain variable region described in (I);
(4) The antibody or antigen-binding fragment of the antibody according to (1), wherein the heavy chain variable region and the light chain variable region are the heavy chain variable region and the light chain variable region described in (III);
(5) The antibody or antigen-binding fragment of the antibody according to any one of (1) to (4), wherein the antibody or antigen-binding fragment is a chimeric antibody or an antigen-binding fragment of the antibody;
(6) The antibody or antigen-binding fragment of the antibody according to any one of (1) to (4), wherein the antibody or antigen-binding fragment is a humanized antibody or an antigen-binding fragment of the antibody;
(7) The antibody or antigen-binding fragment of the antibody according to any one of (1) to (4), wherein the antibody or antigen-binding fragment is a human antibody or an antigen-binding fragment of the antibody;
(8) The antibody or antigen-binding fragment of the antibody according to any one of (1), (3), and (6), wherein the antibody comprises
a light chain variable region comprising any one amino acid sequence represented by
amino acid residues 21 to 127 of the amino acid sequence represented by SEQ ID NO: 64,
amino acid residues 21 to 127 of the amino acid sequence represented by SEQ ID NO: 66,
amino acid residues 21 to 127 of the amino acid sequence represented by SEQ ID NO: 68,
amino acid residues 21 to 127 of the amino acid sequence represented by SEQ ID NO: 70, and
amino acid residues 21 to 127 of the amino acid sequence represented by SEQ ID NO: 72, and
a heavy chain variable region comprising any one amino acid sequence represented by
amino acid residues 20 to 142 of the amino acid sequence represented by SEQ ID NO: 74,
amino acid residues 20 to 142 of the amino acid sequence represented by SEQ ID NO: 76,
amino acid residues 20 to 142 of the amino acid sequence represented by SEQ ID NO: 78, and
amino acid residues 20 to 142 of the amino acid sequence represented by SEQ ID NO: 80;
(9) The antibody or antigen-binding fragment of the antibody according to any one of (1), (3), (6), and (8), wherein the antibody comprises any one combination of a heavy chain variable region and a light chain variable region of
a heavy chain variable region comprising an amino acid sequence represented by amino acid residues 20 to 142 of the amino acid sequence represented by SEQ ID NO: 74, and a light chain variable region comprising an amino acid sequence represented by amino acid residues 21 to 127 of the amino acid sequence represented by SEQ ID NO: 64,
a heavy chain variable region comprising an amino acid sequence represented by amino acid residues 20 to 142 of the amino acid sequence represented by SEQ ID NO: 74, and a light chain variable region comprising an amino acid sequence represented by amino acid residues 21 to 127 of the amino acid sequence represented by SEQ ID NO: 66,
a heavy chain variable region comprising an amino acid sequence represented by amino acid residues 20 to 142 of the amino acid sequence represented by SEQ ID NO: 76, and a light chain variable region comprising an amino acid sequence represented by amino acid residues 21 to 127 of the amino acid sequence represented by SEQ ID NO: 66,
a heavy chain variable region comprising an amino acid sequence represented by amino acid residues 20 to 142 of the amino acid sequence represented by SEQ ID NO: 76, and a light chain variable region comprising an amino acid sequence represented by amino acid residues 21 to 127 of the amino acid sequence represented by SEQ ID NO: 68,
a heavy chain variable region comprising an amino acid sequence represented by amino acid residues 20 to 142 of the amino acid sequence represented by SEQ ID NO: 76, and a light chain variable region comprising an amino acid sequence represented by amino acid residues 21 to 127 of the amino acid sequence represented by SEQ ID NO: 70,
a heavy chain variable region comprising an amino acid sequence represented by amino acid residues 20 to 142 of the amino acid sequence represented by SEQ ID NO: 76, and a light chain variable region comprising an amino acid sequence represented by amino acid residues 21 to 127 of the amino acid sequence represented by SEQ ID NO: 72,
a heavy chain variable region comprising an amino acid sequence represented by amino acid residues 20 to 142 of the amino acid sequence represented by SEQ ID NO: 78, and a light chain variable region comprising an amino acid sequence represented by amino acid residues 21 to 127 of the amino acid sequence represented by SEQ ID NO: 68,
a heavy chain variable region comprising an amino acid sequence represented by amino acid residues 20 to 142 of the amino acid sequence represented by SEQ ID NO: 78, and a light chain variable region comprising an amino acid sequence represented by amino acid residues 21 to 127 of the amino acid sequence represented by SEQ ID NO: 70,
a heavy chain variable region comprising an amino acid sequence represented by amino acid residues 20 to 142 of the amino acid sequence represented by SEQ ID NO: 78, and a light chain variable region comprising an amino acid sequence represented by amino acid residues 21 to 127 of the amino acid sequence represented by SEQ ID NO: 72,
a heavy chain variable region comprising an amino acid sequence represented by amino acid residues 20 to 142 of the amino acid sequence represented by SEQ ID NO: 80, and a light chain variable region comprising an amino acid sequence represented by amino acid residues 21 to 127 of the amino acid sequence represented by SEQ ID NO: 64,
a heavy chain variable region comprising an amino acid sequence represented by amino acid residues 20 to 142 of the amino acid sequence represented by SEQ ID NO: 80, and a light chain variable region comprising an amino acid sequence represented by amino acid residues 21 to 127 of the amino acid sequence represented by SEQ ID NO: 68,
a heavy chain variable region comprising an amino acid sequence represented by amino acid residues 20 to 142 of the amino acid sequence represented by SEQ ID NO: 80, and a light chain variable region comprising an amino acid sequence represented by amino acid residues 21 to 127 of the amino acid sequence represented by SEQ ID NO: 70, and
a heavy chain variable region comprising an amino acid sequence represented by amino acid residues 20 to 142 of the amino acid sequence represented by SEQ ID NO: 80, and a light chain variable region comprising an amino acid sequence represented by amino acid residues 21 to 127 of the amino acid sequence represented by SEQ ID NO: 72;
(10) The antibody or antigen-binding fragment of the antibody according to any one of (1), (4), and (6), wherein the antibody comprises
a light chain variable region comprising an amino acid sequence represented by
amino acid residues 21 to 126 of the amino acid sequence represented by SEQ ID NO: 82, or
amino acid residues 21 to 126 of the amino acid sequence represented by SEQ ID NO: 84, and
a heavy chain variable region comprising any one amino acid sequence represented by
amino acid residues 20 to 142 of the amino acid sequence represented by SEQ ID NO: 86,
amino acid residues 20 to 142 of the amino acid sequence represented by SEQ ID NO: 88,
amino acid residues 20 to 142 of the amino acid sequence represented by SEQ ID NO: 90, and
amino acid residues 20 to 142 of the amino acid sequence represented by SEQ ID NO: 92;
(11) The antibody or antigen-binding fragment of the antibody according to any one of (1), (4), (6), and (10), wherein the antibody comprises any one combination of a heavy chain variable region and a light chain variable region of
a heavy chain variable region comprising an amino acid sequence represented by amino acid residues 20 to 142 of the amino acid sequence represented by SEQ ID NO: 86, and a light chain variable region comprising an amino acid sequence represented by amino acid residues 21 to 126 of the amino acid sequence represented by SEQ ID NO: 84,
a heavy chain variable region comprising an amino acid sequence represented by amino acid residues 20 to 142 of the amino acid sequence represented by SEQ ID NO: 88, and a light chain variable region comprising an amino acid sequence represented by amino acid residues 21 to 126 of the amino acid sequence represented by SEQ ID NO: 84,
a heavy chain variable region comprising an amino acid sequence represented by amino acid residues 20 to 142 of the amino acid sequence represented by SEQ ID NO: 90, and a light chain variable region comprising an amino acid sequence represented by amino acid residues 21 to 126 of the amino acid sequence represented by SEQ ID NO: 82,
a heavy chain variable region comprising an amino acid sequence represented by amino acid residues 20 to 142 of the amino acid sequence represented by SEQ ID NO: 90, and a light chain variable region comprising an amino acid sequence represented by amino acid residues 21 to 126 of the amino acid sequence represented by SEQ ID NO: 84, and
a heavy chain variable region comprising an amino acid sequence represented by amino acid residues 20 to 142 of the amino acid sequence represented by SEQ ID NO: 92, and a light chain variable region comprising an amino acid sequence represented by amino acid residues 21 to 126 of the amino acid sequence represented by SEQ ID NO: 82;
(12) The antibody according to any one of (1) to (11), wherein the antibody comprises Fc;
(13) An antibody or an antigen-binding fragment of the antibody, wherein the antibody binds to human GPRC5D and comprises a heavy chain variable region and a light chain variable region described in any one of the following (A) to (D):
(A)
a heavy chain variable region comprising
heavy chain CDR1 consisting of the amino acid sequence represented by SEQ ID NO: 111,
heavy chain CDR2 consisting of the amino acid sequence represented by SEQ ID NO: 112, and
heavy chain CDR3 consisting of the amino acid sequence represented by SEQ ID NO: 113,
and
a light chain variable region comprising
light chain CDR1 consisting of the amino acid sequence represented by SEQ ID NO: 114,
light chain CDR2 consisting of the amino acid sequence represented by SEQ ID NO: 115, and
light chain CDR3 consisting of the amino acid sequence represented by SEQ ID NO: 116,
(B)
a heavy chain variable region comprising
heavy chain CDR1 consisting of the amino acid sequence represented by SEQ ID NO: 117,
heavy chain CDR2 consisting of the amino acid sequence represented by SEQ ID NO: 118, and
heavy chain CDR3 consisting of the amino acid sequence represented by SEQ ID NO: 119,
and
a light chain variable region comprising
light chain CDR1 consisting of the amino acid sequence represented by SEQ ID NO: 120,
light chain CDR2 consisting of the amino acid sequence represented by SEQ ID NO: 121, and
light chain CDR3 consisting of the amino acid sequence represented by SEQ ID NO: 122,
(C)
a heavy chain variable region comprising
heavy chain CDR1 consisting of the amino acid sequence represented by SEQ ID NO: 123,
heavy chain CDR2 consisting of the amino acid sequence represented by SEQ ID NO: 124, and
heavy chain CDR3 consisting of the amino acid sequence represented by SEQ ID NO: 125,
and
a light chain variable region comprising
light chain CDR1 consisting of the amino acid sequence represented by SEQ ID NO: 126,
light chain CDR2 consisting of the amino acid sequence represented by SEQ ID NO: 127, and
light chain CDR3 consisting of the amino acid sequence represented by SEQ ID NO: 128, and
(D)
a heavy chain variable region comprising
heavy chain CDR1 consisting of the amino acid sequence represented by SEQ ID NO: 129,
heavy chain CDR2 consisting of the amino acid sequence represented by SEQ ID NO: 130, and
heavy chain CDR3 consisting of the amino acid sequence represented by SEQ ID NO: 131,
and
a light chain variable region comprising
light chain CDR1 consisting of the amino acid sequence represented by SEQ ID NO: 132,
light chain CDR2 consisting of the amino acid sequence represented by SEQ ID NO: 133, and
light chain CDR3 consisting of the amino acid sequence represented by SEQ ID NO: 134;
(14) The antibody or antigen-binding fragment of the antibody according to (13), wherein the heavy chain variable region and the light chain variable region are the heavy chain variable region and the light chain variable region described in (A);
(15) The antibody or antigen-binding fragment of the antibody according to (13), wherein the heavy chain variable region and the light chain variable region are the heavy chain variable region and the light chain variable region described in (B);
(16) The antibody or antigen-binding fragment of the antibody according to (13), wherein the heavy chain variable region and the light chain variable region are the heavy chain variable region and the light chain variable region described in (C);
(17) The antibody or antigen-binding fragment of the antibody according to (13), wherein the heavy chain variable region and the light chain variable region are the heavy chain variable region and the light chain variable region described in (D);
(18) The antibody or antigen-binding fragment of the antibody according to any one of (13) to (17), wherein the antibody comprises
a heavy chain variable region comprising any one of
the amino acid sequence represented by SEQ ID NO: 97,
the amino acid sequence represented by SEQ ID NO: 101,
the amino acid sequence represented by SEQ ID NO: 105, and
the amino acid sequence represented by SEQ ID NO: 109,
and
a light chain variable region comprising any one of
the amino acid sequence represented by SEQ ID NO: 99,
the amino acid sequence represented by SEQ ID NO: 103,
the amino acid sequence represented by SEQ ID NO: 107, and
the amino acid sequence represented by SEQ ID NO: 135;
(19) The antibody or antigen-binding fragment of the antibody according to any one of (13) to (18), wherein the antibody comprises any one combination of a heavy chain variable region and a light chain variable region of
a heavy chain variable region comprising the amino acid sequence represented by SEQ ID NO: 97, and a light chain variable region comprising the amino acid sequence represented by SEQ ID NO: 99,
a heavy chain variable region comprising the amino acid sequence represented by SEQ ID NO: 101, and a light chain variable region comprising the amino acid sequence represented by SEQ ID NO: 103,
a heavy chain variable region comprising the amino acid sequence represented by SEQ ID NO: 105, and a light chain variable region comprising the amino acid sequence represented by SEQ ID NO: 107, and
a heavy chain variable region comprising the amino acid sequence represented by SEQ ID NO: 109, and a light chain variable region comprising the amino acid sequence represented by SEQ ID NO: 135;
(20) An antibody or an antigen-binding fragment of the antibody, wherein the antibody comprises any one combination of a heavy chain and a light chain of
a heavy chain comprising the amino acid sequence represented by SEQ ID NO: 144, and a light chain comprising the amino acid sequence represented by SEQ ID NO: 145,
a heavy chain comprising the amino acid sequence represented by SEQ ID NO: 146, and a light chain comprising the amino acid sequence represented by SEQ ID NO: 147,
a heavy chain comprising the amino acid sequence represented by SEQ ID NO: 148, and a light chain comprising the amino acid sequence represented by SEQ ID NO: 149, and
a heavy chain comprising the amino acid sequence represented by SEQ ID NO: 150, and a light chain comprising the amino acid sequence represented by SEQ ID NO: 151;
(21) An antibody or an antigen-binding fragment of the antibody, wherein the antibody or antigen-binding fragment comprises an amino acid sequence encoded by a nucleotide sequence contained in a polynucleotide hybridizing under stringent conditions to a complementary strand of a polynucleotide comprising a nucleotide sequence encoding an amino acid sequence contained in an antibody or an antigen-binding fragment of the antibody according to any one of (8) to (12) and (18) to (20), and binds to human GPRC5D;
(22) An antibody or an antigen-binding fragment of the antibody, wherein the antibody or antigen-binding fragment comprises an amino acid sequence 90% or more identical to an amino acid sequence contained in an antibody or an antigen-binding fragment of the antibody according to any one of (8) to (12) and (18) to (20), and binds to human GPRC5D;
(23) An antibody or an antigen-binding fragment of the antibody, wherein the antibody or antigen-binding fragment comprises an amino acid sequence derived by the substitution, deletion, or addition of 1 to several amino acid(s) from an amino acid sequence contained in an antibody or an antigen-binding fragment of the antibody according to any one of (8) to (12) and 18 to 20, and binds to human GPRC5D;
(24) An antibody or an antigen-binding fragment of the antibody, wherein the antibody or antigen-binding fragment binds to a site on human GPRC5D bound by an antibody or an antigen-binding fragment of the antibody according to any one of (1) to (20);
(25) An antibody or an antigen-binding fragment of the antibody, wherein the antibody or antigen-binding fragment competes with an antibody or an antigen-binding fragment of the antibody according to any one of (1) to (20) for binding to human GPRC5D.
(26) The antibody or antigen-binding fragment according to any one of (1) to (25), wherein the antibody or antigen-binding fragment binds to cynomolgus monkey GPRC5D;
(27) The antibody or antigen-binding fragment of the antibody according to any one of (1) to (26), wherein the antigen-binding fragment is Fab, F(ab)′, Fv, scFv, or sdAb;
(28) An antibody or an antigen-binding fragment of the antibody, wherein the antibody comprises a heavy chain variable region and a light chain variable region described according to (2),(8) or (9), and comprising
i) a heavy chain constant region comprising an amino acid sequence represented by amino acid residues 147 to 475 of the amino acid sequence represented by SEQ ID NO: 199, and a light chain constant region comprising an amino acid sequence represented by amino acid residues 131 to 237 of the amino acid sequence represented by SEQ ID NO: 203,
ii) a heavy chain constant region comprising an amino acid sequence represented by amino acid residues 147 to 475 of the amino acid sequence represented by SEQ ID NO: 201, and a light chain constant region comprising an amino acid sequence represented by amino acid residues 131 to 237 of the amino acid sequence represented by SEQ ID NO: 205,
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217.
(29) An antibody or an antigen-binding fragment of the antibody, wherein the antibody comprises a heavy chain variable region comprising an amino acid sequence represented by amino acid residues 20 to 142 of the amino acid sequence represented by SEQ ID NO: and a light chain variable region comprising an amino acid sequence represented by amino acid residues 21 to 127 of the amino acid sequence represented by SEQ ID NO: 72, and comprising
i) a heavy chain constant region comprising an amino acid sequence represented by amino acid residues 147 to 475 of the amino acid sequence represented by SEQ ID NO: 199, and a light chain constant region comprising an amino acid sequence represented by amino acid residues 131 to 237 of the amino acid sequence represented by SEQ ID NO: 203,
ii) a heavy chain constant region comprising an amino acid sequence represented by amino acid residues 147 to 475 of the amino acid sequence represented by SEQ ID NO: 201, and a light chain constant region comprising an amino acid sequence represented by amino acid residues 131 to 237 of the amino acid sequence represented by SEQ ID NO: 205,
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217.
(30) An antibody or an antigen-binding fragment of the antibody, wherein the antibody comprises a heavy chain variable region and a light chain variable region described according to (2), (8) or (9), and comprising mutated Fc.
(31) An antibody or an antigen-binding fragment of the antibody, wherein the antibody comprises a heavy chain variable region comprising an amino acid sequence represented by amino acid residues 20 to 142 of the amino acid sequence represented by SEQ ID NO: 76 and a light chain variable region comprising an amino acid sequence represented by amino acid residues 21 to 127 of the amino acid sequence represented by SEQ ID NO: 72, and comprising mutated Fc.
(32) A polynucleotide encoding an antibody or an antigen-binding fragment of the antibody according to any one of (1) to (31);
(33) A vector comprising any one of polynucleotides according to (32);
(34) A cell comprising any one of polynucleotides according to (32) or a vector according to (33), or producing an antibody or an antigen-binding fragment of the antibody according to any one of (1) to (31);
(35) An artificial immunocyte comprising a polynucleotide according to (32) or a vector according to (33), or expressing an antibody or an antigen-binding fragment of the antibody according to any one of (1) to (31) on the cell surface;
(36) A method for producing an antibody or an antigen-binding fragment of the antibody which binds to human GPRC5D and cynomolgus monkey GPRC5D, comprising the steps of: culturing a cell according to (34); and recovering an antibody or an antigen-binding fragment of the antibody which binds to human GPRC5D from the cultures;
(37) An antibody or an antigen-binding fragment of the antibody which binds to human GPRC5D, the antibody or antigen-binding fragment being obtained by a method according to (37);
(38) A pharmaceutical composition for treatment and/or prevention comprising an antibody or an antigen-binding fragment of the antibody according to any one of (1) to (31) and (37), a polynucleotide according to (32), a vector according to (33), or an artificial immunocyte according to (35) as an active ingredient;
(39) The pharmaceutical composition according to (38), wherein the pharmaceutical composition is for the treatment and/or prevention of a cancer;
(40) The pharmaceutical composition according to (39), wherein the cancer is breast cancer, endometrial cancer, ovary cancer, lung cancer, stomach cancer, prostate cancer, kidney cancer, liver cancer, pancreatic cancer, colorectal cancer, esophageal cancer, urinary bladder cancer, uterine cervix cancer, blood cancer, lymphoma, or malignant melanoma expressing a GPRC5D protein;
(41) The pharmaceutical composition according to (40), wherein the cancer is multiple myeloma expressing a GPRC5D protein;
(42) A molecule having antigen binding activity, comprising an antibody or an antigen-binding fragment of the antibody according to any one of (1) to (31) and (37);
(43) The molecule according to (42), wherein the molecule is multispecific;
(44) The molecule according to (42) or (43), wherein the molecule comprises an antibody that binds to human GPRC5D or an antigen-binding fragment of the antibody according to any one of (1) to (31) and (37), and
an antibody or an antigen-binding fragment of the antibody that comprises
a heavy chain variable region comprising
the amino acid sequence of heavy chain CDR1 represented by SEQ ID NO: 183,
the amino acid sequence of heavy chain CDR2 represented by SEQ ID NO: 238, and
the amino acid sequence of heavy chain CDR3 represented by SEQ ID NO: 185, and
a light chain variable region comprising
the amino acid sequence of light chain CDR1 represented by SEQ ID NO: 186,
the amino acid sequence of light chain CDR2 represented by SEQ ID NO: 239, and
the amino acid sequence of light chain CDR3 represented by SEQ ID NO: 188,
and binds to human CD3 and cynomolgus monkey CD3;
(45) The molecule according to (44), wherein, in the heavy chain CDR2, the first Xaa is selected from the group consisting of (A, E, G, H, I, L, T, V, R, and S), and the second Xaa is S; or the first Xaa is N, and the second Xaa is selected from the group consisting of (E, R, F, Y, L, V, I, K, and T), and in the light chain CDR2, Xaa is selected from the group consisting of (Q, A, G, S, N, and D)
and binds to human CD3 and cynomolgus monkey CD3;
(46) The molecule according to (44) or (45), wherein, in the heavy chain CDR2, the first Xaa is selected from the group consisting of (R and S), and the second Xaa is S, and in the light chain CDR2, Xaa is selected from the group consisting of (Q, A, G, S, N, and D), and binds to human CD3 and cynomolgus monkey CD3;
(47) The molecule according to any one of (42) to (45), wherein the antibody or antigen-binding fragment of the antibody comprises a heavy chain variable region comprising the amino acid sequence represented by SEQ ID NO: 240 and a light chain variable region comprising an amino acid sequence represented by any one of SEQ ID NOs: 241, 242, and 243;
and in the amino acid sequence represented by SEQ ID NO: 240, the first Xaa is selected from the group consisting of (A, E, G, H, I, L, T, V, R, and S), and the second Xaa is S; or the first Xaa is N, and the second Xaa is selected from the group consisting of (E, R, F, Y, L, V, I, K, and T),
and in the amino acid sequence represented by any one of SEQ ID NOs: 241, 242, and 243, Xaa is selected from the group consisting of (Q, A, G, S, N, and D);
(48) The molecule according to (47), wherein the first Xaa is selected from the group consisting of (R and S), and the second Xaa is S in SEQ ID NO: 240, and Xaa is selected from the group consisting of (Q, A, G, S, N, and D) in any one of SEQ ID NOs: 241, 242, and 243;
(49) The molecule according to (42) or (44), wherein the molecule comprises an antibody that binds to human GPRC5D or an antigen-binding fragment of the antibody according to any one of (1) to (31) and (37), and
an antibody or an antigen-binding fragment of the antibody that comprises
a heavy chain variable region comprising
the amino acid sequence of heavy chain CDR1 represented by SEQ ID NO: 183,
the amino acid sequence of heavy chain CDR2 represented by SEQ ID NO: 184, and
the amino acid sequence of heavy chain CDR3 represented by SEQ ID NO: 185, and
a light chain variable region comprising
the amino acid sequence of light chain CDR1 represented by SEQ ID NO: 186,
the amino acid sequence of light chain CDR2 represented by SEQ ID NO: 187, and
the amino acid sequence of light chain CDR3 represented by SEQ ID NO: 188,
and binds to human CD3 and cynomolgus monkey CD3;
(50) The molecule according to (49), wherein the antibody that binds to human CD3 and cynomolgus monkey CD3 or antigen-binding fragment of the antibody is an antibody or an antigen-binding fragment of the antibody comprising a heavy chain variable region comprising the amino acid sequence represented by SEQ ID NO: 155 and a light chain variable region comprising an amino acid sequence represented by any one of SEQ ID NOs: 156, 181, and 183;
(51) The molecule according to any one of (44) to (50), wherein the antigen-binding fragment of the antibody that binds to human CD3 and cynomolgus monkey CD3 is Fab, F(ab)′, Fv, scFv, or sdAb;
(52) The molecule according to any one of (44) to (51), wherein the antibody that binds to human CD3 and cynomolgus monkey CD3 is a humanized antibody or a human antibody comprising a human immunoglobulin constant region, Fc or mutated Fc;
(53) The molecule according to any one of (44) to (52), wherein the antibody that binds to human CD3 and cynomolgus monkey CD3 or antigen-binding fragment of the antibody is an antibody or an antigen-binding fragment of the antibody comprising an amino acid sequence represented by any one of SEQ ID NOs: 180, 181, and 182;
(54) The molecule according to any one of (40) to (44), wherein the antibody that binds to human CD3 and cynomolgus monkey CD3 or antigen-binding fragment of the antibody is bound with the antibody or antigen-binding fragment of the antibody according to any one of (1) to (31) and (37) via a linker or without a linker;
(55) The molecule according to any one of (42) to (44) wherein the molecule comprises an antibody that binds to human GPRC5D or an antigen-binding fragment of the antibody according to (2), (8) or (9), and
an antibody that binds to human CD3 and cynomolgus monkey CD3 or an antigen-binding fragment of the antibody comprising
   a heavy chain variable region comprising an amino acid sequence represented by amino acid residues 25 to 142 of the amino acid sequence represented by SEQ ID NO: 207 and a light chain variable region comprising an amino acid sequence represented by amino acid residues 24 to 132 of the amino acid sequence represented by SEQ ID NO: 209,
   a heavy chain variable region comprising an amino acid sequence represented by amino acid residues 25 to 142 of the amino acid sequence represented by SEQ ID NO: 211 and a light chain variable region comprising an amino acid sequence represented by amino acid residues 24 to 130 of the amino acid sequence represented by SEQ ID NO: 213,
   a heavy chain variable region comprising an amino acid sequence represented by amino acid residues 2 to 119 of the amino acid sequence represented by SEQ ID NO: 244 and a light chain variable region comprising an amino acid sequence represented by amino acid residues 135 to 243 of the amino acid sequence represented by SEQ ID NO: 244,
   a heavy chain variable region comprising an amino acid sequence represented by amino acid residues 2 to 119 of the amino acid sequence represented by SEQ ID NO: 245 and a light chain variable region comprising an amino acid sequence represented by amino acid residues 135 to 241 of the amino acid sequence represented by SEQ ID NO: 245,
   a heavy chain variable region comprising an amino acid sequence represented by amino acid residues 2 to 119 of the amino acid sequence represented by SEQ ID NO: 246 and a light chain variable region comprising an amino acid sequence represented by amino acid residues 135 to 243 of the amino acid sequence represented by SEQ ID NO: 246,
   a heavy chain variable region comprising an amino acid sequence represented by amino acid residues 2 to 119 of the amino acid sequence represented by SEQ ID NO: 247 and a light chain variable region comprising an amino acid sequence represented by amino acid residues 135 to 243 of the amino acid sequence represented by SEQ ID NO: 247,
   a heavy chain variable region comprising an amino acid sequence represented by amino acid residues 2 to 119 of the amino acid sequence represented by SEQ ID NO: 248 and a light chain variable region comprising an amino acid sequence represented by amino acid residues 135 to 243 of the amino acid sequence represented by SEQ ID NO: 248,
   a heavy chain variable region comprising an amino acid sequence represented by amino acid residues 2 to 119 of the amino acid sequence represented by SEQ ID NO: 249 and a light chain variable region comprising an amino acid sequence represented by amino acid residues 135 to 243 of the amino acid sequence represented by SEQ ID NO: 249,
   a heavy chain variable region comprising an amino acid sequence represented by amino acid residues 2 to 119 of the amino acid sequence represented by SEQ ID NO: 250 and a light chain variable region comprising an amino acid sequence represented by amino acid residues 135 to 243 of the amino acid sequence represented by SEQ ID NO: 250,
   a heavy chain variable region comprising an amino acid sequence represented by amino acid residues 2 to 119 of the amino acid sequence represented by SEQ ID NO: 251 and a light chain variable region comprising an amino acid sequence represented by amino acid residues 135 to 243 of the amino acid sequence represented by SEQ ID NO: 251,
   a heavy chain variable region comprising an amino acid sequence represented by amino acid residues 2 to 119 of the amino acid sequence represented by SEQ ID NO: 252 and a light chain variable region comprising an amino acid sequence represented by amino acid residues 135 to 243 of the amino acid sequence represented by SEQ ID NO: 252,
   a heavy chain variable region comprising an amino acid sequence represented by amino acid residues 2 to 119 of the amino acid sequence represented by SEQ ID NO: 253 and a light chain variable region comprising an amino acid sequence represented by amino acid residues 135 to 243 of the amino acid sequence represented by SEQ ID NO: 253,
   a heavy chain variable region comprising an amino acid sequence represented by amino acid residues 2 to 119 of the amino acid sequence represented by SEQ ID NO: 254 and a light chain variable region comprising an amino acid sequence represented by amino acid residues 135 to 243 of the amino acid sequence represented by SEQ ID NO: 254, or
   a heavy chain variable region comprising an amino acid sequence represented by amino acid residues 2 to 119 of the amino acid sequence represented by SEQ ID NO: 255 and a light chain variable region comprising an amino acid sequence represented by amino acid residues 135 to 243 of the amino acid sequence represented by SEQ ID NO: 255;
(56) The molecule according to (55) wherein the antibody that binds to human GPRC5D or an antigen-binding fragment of the antibody comprises a heavy chain variable region comprising an amino acid sequence represented by amino acid residues 20 to 142 of the amino acid sequence represented by SEQ ID NO: and a light chain variable region comprising an amino acid sequence represented by amino acid residues 21 to 127 of the amino acid sequence represented by SEQ ID NO: 72, and comprises
i) a heavy chain constant region comprising an amino acid sequence represented by amino acid residues 147 to 475 of the amino acid sequence represented by SEQ ID NO: 199, and a light chain constant region comprising an amino acid sequence represented by amino acid residues 131 to 237 of the amino acid sequence represented by SEQ ID NO: 203,
ii) a heavy chain constant region comprising an amino acid sequence represented by amino acid residues 147 to 475 of the amino acid sequence represented by SEQ ID NO: 201, and a light chain constant region comprising an amino acid sequence represented by amino acid residues 131 to 237 of the amino acid sequence represented by SEQ ID NO: 205,
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217,
and
the antibody that binds to human CD3 and cynomolgus monkey CD3 or antigen-binding fragment of the antibody comprises mutated Fc;
(57) The molecule according to (55) wherein the antibody that binds to human GPRC5D or an antigen-binding fragment of the antibody comprises a heavy chain variable region comprising an amino acid sequence represented by amino acid residues 20 to 142 of the amino acid sequence represented by SEQ ID NO: 76 and a light chain variable region comprising an amino acid sequence represented by amino acid residues 21 to 127 of the amino acid sequence represented by SEQ ID NO: 72, and comprises
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217,
and
the antibody that binds to human CD3 and cynomolgus monkey CD3 or antigen-binding fragment of the antibody comprises mutated Fc;
(58) The molecule according to (55) which comprises

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 219 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 221 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 225 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 227 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 229 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 231 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 233 and mutated Fc; or

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 235 and mutated Fc;
(59) The molecule according to (55) which comprises

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 225 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 227 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 229 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 231 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 233 and mutated Fc; or

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 235 and mutated Fc;
(60) The molecule according to (55) wherein the antibody that binds to human GPRC5D or an antigen-binding fragment of the antibody comprises a heavy chain variable region comprising an amino acid sequence represented by amino acid residues 20 to 142 of the amino acid sequence represented by SEQ ID NO: and a light chain variable region comprising an amino acid sequence represented by amino acid residues 21 to 127 of the amino acid sequence represented by SEQ ID NO: 72, and comprises
i) a heavy chain constant region comprising an amino acid sequence represented by amino acid residues 147 to 475 of the amino acid sequence represented by SEQ ID NO: 199, and a light chain constant region comprising an amino acid sequence represented by amino acid residues 131 to 237 of the amino acid sequence represented by SEQ ID NO: 203,
ii) a heavy chain constant region comprising an amino acid sequence represented by amino acid residues 147 to 475 of the amino acid sequence represented by SEQ ID NO: 201, and a light chain constant region comprising an amino acid sequence represented by amino acid residues 131 to 237 of the amino acid sequence represented by SEQ ID NO: 205,
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217,
and
the antibody that binds to human CD3 and cynomolgus monkey CD3 or antigen-binding fragment of the antibody comprises
v) a heavy chain constant region comprising an amino acid sequence represented by amino acid residues 147 to 471 of the amino acid sequence represented by SEQ ID NO: 207, and a light chain constant region comprising an amino acid sequence represented by amino acid residues 133 to 238 of the amino acid sequence represented by SEQ ID NO: 209, or
vi) a heavy chain constant region comprising an amino acid sequence represented by amino acid residues 143 to 471 of the amino acid sequence represented by SEQ ID NO: 211, and a light chain constant region comprising an amino acid sequence represented by amino acid residues 131 to 236 of the amino acid sequence represented by SEQ ID NO: 213;
(62) The molecule according to (55) which comprises

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 119 and a light chain comprising an amino acid sequence represented by amino acid residues 24 to 237 of the amino acid sequence represented by SEQ ID NO: 203, and
the antibody that binds to human CD3 and cynomolgus monkey CD3 or antigen-binding fragment of the antibody comprising a heavy chain comprising an amino acid sequence represented by amino acid residues 25 to 471 of the amino acid sequence represented by SEQ ID NO: 207 and a light chain comprising an amino acid sequence represented by amino acid residues 24 to 238 of the amino acid sequence represented by SEQ ID NO: 209
or
the antibody that binds to human GPRC5D or an antigen-binding fragment of the antibody comprising a heavy chain comprising an amino acid sequence represented by amino acid residues 24 to 475 of the amino acid sequence represented by SEQ ID NO: 201 and a light chain comprising an amino acid sequence represented by amino acid residues 24 to 237 of the amino acid sequence represented by SEQ ID NO: 205, and
the antibody that binds to human CD3 and cynomolgus monkey CD3 or antigen-binding fragment of the antibody comprising a heavy chain comprising an amino acid sequence represented by amino acid residues 25 to 471 of the amino acid sequence represented by SEQ ID NO: 211 and a light chain comprising an amino acid sequence represented by amino acid residues 24 to 236 of the amino acid sequence represented by SEQ ID NO: 213;
(63) The molecule according to (55) wherein the antibody that binds to human GPRC5D or an antigen-binding fragment of the antibody comprises a heavy chain variable region comprising an amino acid sequence represented by amino acid residues 20 to 142 of the amino acid sequence represented by SEQ ID NO: 76 and a light chain variable region comprising an amino acid sequence represented by amino acid residues 21 to 127 of the amino acid sequence represented by SEQ ID NO: 72, and mutated Fc, and the antibody that binds to human CD3 and cynomolgus monkey CD3 or antigen-binding fragment of the antibody comprises mutated Fc;
(64) The molecule according to (55) which comprises

the antibody that binds to human GPRC5D or an antigen-binding fragment of the antibody comprising amino acid sequence represented by amino acid residues 24 to 271 of the amino acid sequence represented by SEQ ID NO: 223 and mutated Fc, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 219 and mutated Fc
or
the antibody that binds to human GPRC5D or an antigen-binding fragment of the antibody comprising amino acid sequence represented by amino acid residues 24 to 271 of the amino acid sequence represented by SEQ ID NO: 223 and mutated Fc, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 221 and mutated Fc;
(65) The molecule according to (53), wherein the antibody that binds to human CD3 and cynomolgus monkey CD3 or antigen-binding fragment of the antibody is bound with the antibody or antigen-binding fragment of the antibody according to (19) via a linker or without a linker;
(66) The molecule according to (54) or (65), wherein the molecule has an amino acid sequence represented by any one of SEQ ID NOs: 171 to 179 and binds to human CD3 and cynomolgus monkey CD3 and to human GPRC5D;
(67) A molecule which comprises an amino acid sequence encoded by a nucleotide sequence contained in a polynucleotide hybridizing under stringent conditions to a complementary strand of a polynucleotide comprising a nucleotide sequence encoding an amino acid sequence contained in an antibody that binds to human CD3 and cynomolgus monkey CD3 or an antigen-binding fragment of the antibody contained in a molecule according to any one of (50), (53), (58), (59), (62), (64), (65) and (66), and binds to human CD3 and cynomolgus monkey CD3 and to human GPRC5D;
(68) A molecule which comprises an amino acid sequence 90% or more identical to an amino acid sequence contained in an antibody that binds to human CD3 and cynomolgus monkey CD3 or an antigen-binding fragment of the antibody according to any one of (50), (53), (58), (59), (62), (64), (65) and (66), and binds to human CD3 and cynomolgus monkey CD3 and to human GPRC5D;
(69) A molecule which comprises an amino acid sequence derived by the substitution, deletion, or addition of 1 to several amino acid(s) from an amino acid sequence contained in an antibody that binds to human CD3 and cynomolgus monkey CD3 or an antigen-binding fragment of the antibody contained in a molecule according to any one of (50), (53), (58), (59), (62), (64), (65) and (66), and binds to human CD3 and cynomolgus monkey CD3 and to human GPRC5D;
(70) The molecule according to any one of (42) to (69), wherein the molecule binds to cynomolgus monkey GPRC5D;
(71) The molecule according to any one of (43) to (70), wherein the molecule is bispecific;
(72) The molecule according to any one of (42) to (71), wherein the molecule is a polypeptide;
(73) A polynucleotide comprising a nucleotide sequence encoding the amino acid sequence of a molecule according to (72);
(74) A vector comprising a polynucleotide according to (73);
(75) A cell producing a polynucleotide according to (73) or a vector according to (74), or a molecule according to (71);
(76) A method for producing a molecule binding to human CD3 and cynomolgus monkey CD3 and to human GPRC5D, comprising the steps of: culturing a cell according to (75); and recovering a molecule binding to human CD3 and cynomolgus monkey CD3 and/or to human GPRC5D from the cultures;
(77) A molecule binding to human CD3 and cynomolgus monkey CD3 and to human GPRC5D, the molecule being obtained by a method according to (76);
(78) The molecule according to (77), wherein the molecule binds to cynomolgus monkey GPRC5D;
(79) A pharmaceutical composition for treatment and/or prevention comprising a molecule according to any one of (42) to (72), (77), and (78), a polynucleotide according to (73), or a vector according to (74) as an active ingredient;
(80) The pharmaceutical composition according to (79), wherein the pharmaceutical composition is for the treatment and/or prevention of a cancer;
(81) The pharmaceutical composition according to (80), wherein the cancer is breast cancer, endometrial cancer, ovary cancer, lung cancer, stomach cancer, prostate cancer, kidney cancer, liver cancer, pancreatic cancer, colorectal cancer, esophageal cancer, urinary bladder cancer, uterine cervix cancer, blood cancer, lymphoma, or malignant melanoma expressing a GPRC5D protein;
(82) The pharmaceutical composition according to (79) or (80), wherein the cancer is multiple myeloma expressing a GPRC5D protein;
(83) A method for treating and/or preventing a cancer, comprising administering a molecule according to any one of (42) to (72), (77), and (78) or a pharmaceutical composition according to any one of (79) to (82);
(84) The pharmaceutical composition according to any one of (79) to (82), wherein the pharmaceutical composition induces cytotoxicity to cells expressing GPRC5D by the redirection of T cells to the cells;
(85) The method according to (83), wherein the method induces cytotoxicity to cells expressing GPRC5D by the redirection of T cells to the cells;
(86) A method for inducing cytotoxicity to cells expressing GPRC5D by the redirection of T cells to the cells, comprising the step of administering a molecule according to any one of (42) to (72), (77), and (78) or a pharmaceutical composition according to any one of (79) to (82); and
(87) A method for redirecting T cells to cells expressing GPRC5D, comprising the step of administering a molecule according to any one of (42) to (72), (77), and (78) or a pharmaceutical composition according to any one of (79) to (82).

### Advantageous Effects of Invention

According to the present invention, a novel anti-GPRC5D antibody or an antigen-binding fragment of the antibody which binds to human GPRC5D, and a novel molecule comprising the antibody or antigen-binding fragment of the antibody and having antigen binding activity, are obtained. The molecule can comprise an anti-CD3 antibody.

Use of the antibody or antigen-binding fragment of the antibody, and the molecule provided by the present invention allows for treatment or prevention of various cancers, preferably, multiple myeloma, expressing a GPRC5D protein.

### BRIEF DESCRIPTION OF DRAWINGS

FIG. 1 is a diagram showing results of testing the binding activity of rat anti-GPRC5D antibodies (2A4, 2B1, and 7B4) against human GPRC5D by flow cytometry. The vertical axis represents a relative value of the mean fluorescence intensity assayed by flow cytometry.

FIG. 2 is a diagram showing the amino-terminal amino acid sequence of human GPRC5D (SEQ ID NO: 1 of the Sequence Listing).

FIG. 3 is a diagram showing the amino-terminal amino acid sequence of human GPRC5D (SEQ ID NO: 2 of the Sequence Listing).

FIG. 4 is a diagram showing results of testing the binding activity of the rat anti-GPRC5D antibodies (2A4, 2B1, and 7B4) against human GPRC5D using a flow cytometer (FACS). The vertical axis represents a relative value of the mean fluorescence intensity assayed by flow cytometry. The intramolecular disulfide bond of a peptide was present (A) or absent (B).

FIG. 5 is a diagram showing that the rat anti-GPRC5D antibodies (2A4, 2B1, and 7B4) have ADCC activity.

FIG. 6 is a diagram showing the nucleotide sequence of a primer for the PCR amplification of the variable region-encoding cDNA of the heavy chain gene of 2A4 (SEQ ID NO: 3 of the Sequence Listing).

FIG. 7 is a diagram showing the nucleotide sequence of a primer for the PCR amplification of the variable region-encoding cDNA of the light chain gene of 2A4 (SEQ ID NO: 10 of the Sequence Listing).

FIG. 8 is a diagram showing the nucleotide sequence of a cDNA encoding the heavy chain variable region of 2A4 (SEQ ID NO: 4 of the Sequence Listing).

FIG. 9 is a diagram showing the amino acid sequence of the heavy chain variable region of 2A4 (SEQ ID NO: 5 of the Sequence Listing).

FIG. 10 is a diagram showing the nucleotide sequence of a cDNA encoding the heavy chain variable region of 2B1 (SEQ ID NO: 6 of the Sequence Listing).

FIG. 11 is a diagram showing the amino acid sequence of the heavy chain variable region of 2B1 (SEQ ID NO: 7 of the Sequence Listing).

FIG. 12 is a diagram showing the nucleotide sequence of a cDNA encoding the heavy chain variable region of 7B4 (SEQ ID NO: 8 of the Sequence Listing).

FIG. 13 is a diagram showing the amino acid sequence of the heavy chain variable region of 7B4 (SEQ ID NO: 9 of the Sequence Listing).

FIG. 14 is a diagram showing the nucleotide sequence of a cDNA encoding the light chain variable region of 2A4 (SEQ ID NO: 11 of the Sequence Listing).

FIG. 15 is a diagram showing the amino acid sequence of the light chain variable region of 2A4 (SEQ ID NO: 12 of the Sequence Listing).

FIG. 16 is a diagram showing the nucleotide sequence of a cDNA encoding the light chain variable region of 2B1 (SEQ ID NO: 13 of the Sequence Listing).

FIG. 17 is a diagram showing the amino acid sequence of the light chain variable region of 2B1 (SEQ ID NO: 14 of the Sequence Listing).

FIG. 18 is a diagram showing the nucleotide sequence of a cDNA encoding the light chain variable region of 7B4 (SEQ ID NO: 15 of the Sequence Listing).

FIG. 19 is a diagram showing the amino acid sequence of the light chain variable region of 7B4 (SEQ ID NO: 16 of the Sequence Listing).

FIG. 20 is a diagram showing the nucleotide sequence of a DNA fragment comprising a DNA sequence encoding the amino acids of a human κ chain secretory signal sequence and a human κ chain constant region (SEQ ID NO: 17 of the Sequence Listing).

FIG. 21 is a diagram showing the nucleotide sequence of a primer F for a light chain expression vector (SEQ ID NO: 18 of the Sequence Listing).

FIG. 22 is a diagram showing the nucleotide sequence of a primer R for the light chain expression vector (SEQ ID NO: 19 of the Sequence Listing).

FIG. 23 is a diagram showing the nucleotide sequence of a DNA fragment comprising a DNA sequence encoding the amino acids of a human heavy chain signal sequence and a human IgG1 constant region (SEQ ID NO: 20 of the Sequence Listing).

FIG. 24 is a diagram showing the nucleotide sequence of the light chain of human chimeric 2A4 (c2A4) (SEQ ID NO: 21 of the Sequence Listing).

FIG. 25 is a diagram showing the amino acid sequence of the light chain of human chimeric 2A4 (c2A4) (signal sequence (amino acid positions 1 to 20), variable region (amino acid positions 21 to 127), constant region (amino acid positions 128 to 234)) (SEQ ID NO: 22 of the Sequence Listing).

FIG. 26 is a diagram showing the nucleotide sequence of a primer set F for the light chain of human chimeric 2A4 (c2A4) (SEQ ID NO: 23 of the Sequence Listing).

FIG. 27 is a diagram showing the nucleotide sequence of a primer set R for the light chain of human chimeric 2A4 (c2A4) (SEQ ID NO: 24 of the Sequence Listing).

FIG. 28 is a diagram showing the nucleotide sequence of the heavy chain of human chimeric 2A4 (c2A4) (SEQ ID NO: 25 of the Sequence Listing).

FIG. 29 is a diagram showing the amino acid sequence of the heavy chain of human chimeric 2A4 (c2A4) (signal sequence (amino acid positions 1 to 19), variable region (amino acid positions 20 to 141), constant region (amino acid positions 142 to 471)) (SEQ ID NO: 26 of the Sequence Listing).

FIG. 30 is a diagram showing the nucleotide sequence of a primer set F for the heavy chain of human chimeric 2A4 (c2A4) (SEQ ID NO: 27 of the Sequence Listing).

FIG. 31 is a diagram showing the nucleotide sequence of a primer set R for the heavy chain of human chimeric 2A4 (c2A4) (SEQ ID NO: 28 of the Sequence Listing).

FIG. 32 is a diagram showing the nucleotide sequence of the light chain of human chimeric 2B1 (c2B1) (SEQ ID NO: 29 of the Sequence Listing).

FIG. 33 is a diagram showing the amino acid sequence of the light chain of human chimeric 2B1 (c2B1) (signal sequence (amino acid positions 1 to 20), variable region (amino acid positions 21 to 127), constant region (amino acid positions 128 to 234)) (SEQ ID NO: 30 of the Sequence Listing).

FIG. 34 is a diagram showing the nucleotide sequence of a primer set F for the light chain of human chimeric 2B1 (c2B1) (SEQ ID NO: 31 of the Sequence Listing).

FIG. 35 is a diagram showing the nucleotide sequence of a primer set R for the light chain of human chimeric 2B1 (c2B1) (SEQ ID NO: 32 of the Sequence Listing).

FIG. 36 is a diagram showing the nucleotide sequence of the heavy chain of human chimeric 2B1 (c2B1) (SEQ ID NO: 33 of the Sequence Listing).

FIG. 37 is a diagram showing the amino acid sequence of the heavy chain of human chimeric 2B1 (c2B1) (signal sequence (amino acid positions 1 to 19), variable region (amino acid positions 20 to 142), constant region (amino acid positions 143 to 472)) (SEQ ID NO: 34 of the Sequence Listing).

FIG. 38 is a diagram showing the nucleotide sequence of a primer set F for the heavy chain of human chimeric 2B1 (c2B1) (SEQ ID NO: 35 of the Sequence Listing).

FIG. 39 is a diagram showing the nucleotide sequence of a primer set R for the heavy chain of human chimeric 2B1 (c2B1) (SEQ ID NO: 36 of the Sequence Listing).

FIG. 40 is a diagram showing the nucleotide sequence of the light chain of human chimeric 7B4 (c7B4) (SEQ ID NO: 37 of the Sequence Listing).

FIG. 41 is a diagram showing the amino acid sequence of the light chain of human chimeric 7B4 (c7B4) (signal sequence (amino acid positions 1 to 20), variable region (amino acid positions 21 to 126), constant region (amino acid positions 127 to 233)) (SEQ ID NO: 38 of the Sequence Listing).

FIG. 42 is a diagram showing the nucleotide sequence of a primer set F for the light chain of human chimeric 7B4 (c7B4) (SEQ ID NO: 39 of the Sequence Listing).

FIG. 43 is a diagram showing the nucleotide sequence of a primer set R for the light chain of human chimeric 7B4 (c7B4) (SEQ ID NO: 40 of the Sequence Listing).

FIG. 44 is a diagram showing the nucleotide sequence of the heavy chain of human chimeric 7B4 (c7B4) (SEQ ID NO: 41 of the Sequence Listing).

FIG. 45 is a diagram showing the amino acid sequence of the heavy chain of human chimeric 7B4 (c7B4) (signal sequence (amino acid positions 1 to 19), variable region (amino acid positions 20 to 142), constant region (amino acid positions 143 to 472)) (SEQ ID NO: 42 of the Sequence Listing).

FIG. 46 is a diagram showing the nucleotide sequence of a primer set F for the heavy chain of human chimeric 7B4 (c7B4) (SEQ ID NO: 43 of the Sequence Listing).

FIG. 47 is a diagram showing the nucleotide sequence of a primer set R for the heavy chain of human chimeric 7B4 (c7B4) (SEQ ID NO: 44 of the Sequence Listing).

FIG. 48 is a diagram showing results of testing the binding activity of the human chimeric antibodies (c2A4, c2B1, and c7B4) against human GPRC5D using a flow cytometer (FACS). The vertical axis represents a relative value of the mean fluorescence intensity assayed by flow cytometry.

FIG. 49 is a diagram showing results of testing the binding activity of the human chimeric antibodies (c2A4, c2B1, and c7B4) against cynomolgus monkey GPRC5D using a flow cytometer (FACS). The vertical axis represents a relative value of the mean fluorescence intensity assayed by flow cytometry.

FIG. 50 is a diagram showing that the human chimeric antibodies (c2A4, c2B1, and c7B4) have ADCC activity against human GPRC5D.

FIG. 51 is a diagram showing the in vivo tumor growth inhibitory activity of human chimeric 2A4 (c2A4) against GPRC5D-expressing human multiple myeloma cell line KHM-1B-transplanted BALB/c-nu/nu mice.

FIG. 52 is a diagram showing the in vivo tumor growth inhibitory activity of human chimeric 2B1 (c2B1) against GPRC5D-expressing human multiple myeloma cell line KHM-1B-transplanted BALB/c-nu/nu mice.

FIG. 53 is a diagram showing the in vivo tumor growth inhibitory activity of human chimeric 7B4 (c7B4) against GPRC5D-expressing human multiple myeloma cell line KHM-1B-transplanted BALB/c-nu/nu mice.

FIG. 54 is a diagram showing the amino acid sequence of the heavy chain CDR1 of the rat anti-GPRC5D antibody 2A4 (SEQ ID NO: 45 of the Sequence Listing).

FIG. 55 is a diagram showing the amino acid sequence of the heavy chain CDR2 of the rat anti-GPRC5D antibody 2A4 (SEQ ID NO: 46 of the Sequence Listing).

FIG. 56 is a diagram showing the amino acid sequence of the heavy chain CDR3 of the rat anti-GPRC5D antibody 2A4 (SEQ ID NO: 47 of the Sequence Listing).

FIG. 57 is a diagram showing the amino acid sequence of the heavy chain CDR1 of the rat anti-GPRC5D antibody 2B1 (SEQ ID NO: 48 of the Sequence Listing).

FIG. 58 is a diagram showing the amino acid sequence of the heavy chain CDR2 of the rat anti-GPRC5D antibody 2B1 (SEQ ID NO: 49 of the Sequence Listing).

FIG. 59 is a diagram showing the amino acid sequence of the heavy chain CDR3 of the rat anti-GPRC5D antibody 2B1 (SEQ ID NO: 50 of the Sequence Listing).

FIG. 60 is a diagram showing the amino acid sequence of the heavy chain CDR1 of the rat anti-GPRC5D antibody 7B4 (SEQ ID NO: 51 of the Sequence Listing).

FIG. 61 is a diagram showing the amino acid sequence of the heavy chain CDR2 of the rat anti-GPRC5D antibody 7B4 (SEQ ID NO: 52 of the Sequence Listing).

FIG. 62 is a diagram showing the amino acid sequence of the heavy chain CDR3 of the rat anti-GPRC5D antibody 7B4 (SEQ ID NO: 53 of the Sequence Listing).

FIG. 63 is a diagram showing the amino acid sequence of the light chain CDR1 of the rat anti-GPRC5D antibody 2A4 (SEQ ID NO: 54 of the Sequence Listing).

FIG. 64 is a diagram showing the amino acid sequence of the light chain CDR2 of the rat anti-GPRC5D antibody 2A4 (SEQ ID NO: 55 of the Sequence Listing).

FIG. 65 is a diagram showing the amino acid sequence of the light chain CDR3 of the rat anti-GPRC5D antibody 2A4 (SEQ ID NO: 56 of the Sequence Listing).

FIG. 66 is a diagram showing the amino acid sequence of the light chain CDR1 of the rat anti-GPRC5D antibody 2B1 (SEQ ID NO: 57 of the Sequence Listing).

FIG. 67 is a diagram showing the amino acid sequence of the light chain CDR2 of the rat anti-GPRC5D antibody 2B1 (SEQ ID NO: 58 of the Sequence Listing).

FIG. 68 is a diagram showing the amino acid sequence of the light chain CDR3 of the rat anti-GPRC5D antibody 2B1 (SEQ ID NO: 59 of the Sequence Listing).

FIG. 69 is a diagram showing the amino acid sequence of the light chain CDR1 of the rat anti-GPRC5D antibody 7B4 (SEQ ID NO: 60 of the Sequence Listing).

FIG. 70 is a diagram showing the amino acid sequence of the light chain CDR2 of the rat anti-GPRC5D antibody 7B4 (SEQ ID NO: 61 of the Sequence Listing).

FIG. 71 is a diagram showing the amino acid sequence of the light chain CDR3 of the rat anti-GPRC5D antibody 7B4 (SEQ ID NO: 62 of the Sequence Listing).

FIG. 72 is a diagram showing the nucleotide sequence of a humanized 2B1 light chain (h2B1_L1) (SEQ ID NO: 63 of the Sequence Listing).

FIG. 73 is a diagram showing the amino acid sequence of the humanized 2B1 light chain (h2B1_L1) (signal sequence (amino acid positions 1 to 20), variable region (amino acid positions 21 to 127), constant region (amino acid positions 128 to 234)) (SEQ ID NO: 64 of the Sequence Listing).

FIG. 74 is a diagram showing the nucleotide sequence of a humanized 2B1 light chain (h2B1_L2) (SEQ ID NO: 65 of the Sequence Listing).

FIG. 75 is a diagram showing the amino acid sequence of the humanized 2B1 light chain (h2B1_L2) (signal sequence (amino acid positions 1 to 20), variable region (amino acid positions 21 to 127), constant region (amino acid positions 128 to 234)) (SEQ ID NO: 66 of the Sequence Listing).

FIG. 76 is a diagram showing the nucleotide sequence of a humanized 2B1 light chain (h2B1_L3) (SEQ ID NO: 67 of the Sequence Listing).

FIG. 77 is a diagram showing the amino acid sequence of the humanized 2B1 light chain (h2B1_L3) (signal sequence (amino acid positions 1 to 20), variable region (amino acid positions 21 to 127), constant region (amino acid positions 128 to 234)) (SEQ ID NO: 68 of the Sequence Listing).

FIG. 78 is a diagram showing the nucleotide sequence of a humanized 2B1 light chain (h2B1_L4) (SEQ ID NO: 69 of the Sequence Listing).

FIG. 79 is a diagram showing the amino acid sequence of the humanized 2B1 light chain (h2B1_L4) (signal sequence (amino acid positions 1 to 20), variable region (amino acid positions 21 to 127), constant region (amino acid positions 128 to 234)) (SEQ ID NO: 70 of the Sequence Listing).

FIG. 80 is a diagram showing the nucleotide sequence of a humanized 2B1 light chain (h2B1_L5) (SEQ ID NO: 71 of the Sequence Listing).

FIG. 81 is a diagram showing the amino acid sequence of the humanized 2B1 light chain (h2B1_L5) (signal sequence (amino acid positions 1 to 20), variable region (amino acid positions 21 to 127), constant region (amino acid positions 128 to 234)) (SEQ ID NO: 72 of the Sequence Listing).

FIG. 82 is a diagram showing the nucleotide sequence of a humanized 2B1 heavy chain (h2B1_H1) (SEQ ID NO: 73 of the Sequence Listing).

FIG. 83 is a diagram showing the amino acid sequence of the humanized 2B1 heavy chain (h2B1_H1) (signal sequence (amino acid positions 1 to 19), variable region (amino acid positions 20 to 142), constant region (amino acid positions 143 to 472)) (SEQ ID NO: 74 of the Sequence Listing).

FIG. 84 is a diagram showing the nucleotide sequence of a humanized 2B1 heavy chain (h2B1_H2) (SEQ ID NO: 75 of the Sequence Listing).

FIG. 85 is a diagram showing the amino acid sequence of the humanized 2B1 heavy chain (h2B1_H2) (signal sequence (amino acid positions 1 to 19), variable region (amino acid positions 20 to 142), constant region (amino acid positions 143 to 472)) (SEQ ID NO: 76 of the Sequence Listing).

FIG. 86 is a diagram showing the nucleotide sequence of a humanized 2B1 heavy chain (h2B1_H3) (SEQ ID NO: 77 of the Sequence Listing).

FIG. 87 is a diagram showing the amino acid sequence of the humanized 2B1 heavy chain (h2B1_H3) (signal sequence (amino acid positions 1 to 19), variable region (amino acid positions 20 to 142), constant region (amino acid positions 143 to 472)) (SEQ ID NO: 78 of the Sequence Listing).

FIG. 88 is a diagram showing the nucleotide sequence of a humanized 2B1 heavy chain (h2B1_H4) (SEQ ID NO: 79 of the Sequence Listing).

FIG. 89 is a diagram showing the amino acid sequence of the humanized 2B1 heavy chain (h2B1_H4) (signal sequence (amino acid positions 1 to 19), variable region (amino acid positions 20 to 142), constant region (amino acid positions 143 to 472)) (SEQ ID NO: 80 of the Sequence Listing).

FIG. 90 is a diagram showing the nucleotide sequence of a humanized 7B4 light chain (h7B4_L1) (SEQ ID NO: 81 of the Sequence Listing).

FIG. 91 is a diagram showing the amino acid sequence of the humanized 7B4 light chain (h7B4_L1) (signal sequence (amino acid positions 1 to 20), variable region (amino acid positions 21 to 126), constant region (amino acid positions 127 to 233)) (SEQ ID NO: 82 of the Sequence Listing).

FIG. 92 is a diagram showing the nucleotide sequence of a humanized 7B4 light chain (h7B4_L2) (SEQ ID NO: 83 of the Sequence Listing).

FIG. 93 is a diagram showing the amino acid sequence of the humanized 7B4 light chain (h7B4_L2) (signal sequence (amino acid positions 1 to 20), variable region (amino acid positions 21 to 126), constant region (amino acid positions 127 to 233)) (SEQ ID NO: 84 of the Sequence Listing).

FIG. 94 is a diagram showing the nucleotide sequence of a humanized 7B4 heavy chain (h7B4_H1) (SEQ ID NO: 85 of the Sequence Listing).

FIG. 95 is a diagram showing the amino acid sequence of the humanized 7B4 heavy chain (h7B4_H1) (signal sequence (amino acid positions 1 to 19), variable region (amino acid positions 20 to 142), constant region (amino acid positions 143 to 472)) (SEQ ID NO: 86 of the Sequence Listing).

FIG. 96 is a diagram showing the nucleotide sequence of a humanized 7B4 heavy chain (h7B4_H2) (SEQ ID NO: 87 of the Sequence Listing).

FIG. 97 is a diagram showing the amino acid sequence of the humanized 7B4 heavy chain (h7B4_H2) (signal sequence (amino acid positions 1 to 19), variable region (amino acid positions 20 to 142), constant region (amino acid positions 143 to 472)) (SEQ ID NO: 88 of the Sequence Listing).

FIG. 98 is a diagram showing the nucleotide sequence of a humanized 7B4 heavy chain (h7B4_H3) (SEQ ID NO: 89 of the Sequence Listing).

FIG. 99 is a diagram showing the amino acid sequence of the humanized 7B4 heavy chain (h7B4_H3) (signal sequence (amino acid positions 1 to 19), variable region (amino acid positions 20 to 142), constant region (amino acid positions 143 to 472)) (SEQ ID NO: 90 of the Sequence Listing).

FIG. 100 is a diagram showing the nucleotide sequence of a humanized 7B4 heavy chain (h7B4_H5) (SEQ ID NO: 91 of the Sequence Listing).

FIG. 101 is a diagram showing the amino acid sequence of the humanized 7B4 heavy chain (h7B4_H5) (signal sequence (amino acid positions 1 to 19), variable region (amino acid positions 20 to 142), constant region (amino acid positions 143 to 472)) (SEQ ID NO: 92 of the Sequence Listing).

FIG. 102 is a diagram showing results of testing the binding activity of humanized anti-GPRC5D antibodies h2B1 against human GPRC5D using a flow cytometer (FACS). The vertical axis represents a relative value of the mean fluorescence intensity assayed by flow cytometry.

FIG. 103 is a diagram showing results of testing the binding activity of humanized anti-GPRC5D antibodies h7B4 against human GPRC5D using a flow cytometer (FACS). The vertical axis represents a relative value of the mean fluorescence intensity assayed by flow cytometry.

FIG. 104 is a diagram showing that humanized 2B1 and humanized 7B4 have ADCC activity.

FIG. 105 is a diagram showing the amino acid sequence of the amino-terminal peptide of cynomolgus monkey GPRC5D (SEQ ID NO: 93).

FIG. 106 is a diagram showing the nucleotide sequence of a primer A used in the sequence analysis of scFv (SEQ ID NO: 94).

FIG. 107 is a diagram showing the nucleotide sequence of a primer B used in the sequence analysis of scFv (SEQ ID NO: 95).

FIG. 108 is a diagram showing the nucleotide sequence of the heavy chain variable region of a human antibody C2037 (SEQ ID NO: 96 of the Sequence Listing).

FIG. 109 is a diagram showing the amino acid sequence of the heavy chain variable region of the human antibody C2037 (SEQ ID NO: 97 of the Sequence Listing).

FIG. 110 is a diagram showing the nucleotide sequence of the light chain variable region of a human antibody C2037 (SEQ ID NO: 98 of the Sequence Listing).

FIG. 111 is a diagram showing the amino acid sequence of the light chain variable region of the human antibody C2037 (SEQ ID NO: 99 of the Sequence Listing).

FIG. 112 is a diagram showing the nucleotide sequence of the heavy chain variable region of a human antibody C3048 (SEQ ID NO: 100 of the Sequence Listing).

FIG. 113 is a diagram showing the amino acid sequence of the heavy chain variable region of the human antibody C3048 (SEQ ID NO: 101 of the Sequence Listing).

FIG. 114 is a diagram showing the nucleotide sequence of the light chain variable region of a human antibody C3048 (SEQ ID NO: 102 of the Sequence Listing).

FIG. 115 is a diagram showing the amino acid sequence of the light chain variable region of the human antibody C3048 (SEQ ID NO: 103 of the Sequence Listing).

FIG. 116 is a diagram showing the nucleotide sequence of the heavy chain variable region of a human antibody C3015 (SEQ ID NO: 104 of the Sequence Listing).

FIG. 117 is a diagram showing the amino acid sequence of the heavy chain variable region of the human antibody C3015 (SEQ ID NO: 105 of the Sequence Listing).

FIG. 118 is a diagram showing the nucleotide sequence of the light chain variable region of the human antibody C3015 (SEQ ID NO: 106 of the Sequence Listing).

FIG. 119 is a diagram showing the amino acid sequence of the light chain variable region of the human antibody C3015 (SEQ ID NO: 107 of the Sequence Listing).

FIG. 120 is a diagram showing the nucleotide sequence of the heavy chain variable region of a human antibody C3022 (SEQ ID NO: 108 of the Sequence Listing).

FIG. 121 is a diagram showing the amino acid sequence of the heavy chain variable region of the human antibody C3022 (SEQ ID NO: 109 of the Sequence Listing).

FIG. 122 is a diagram showing the nucleotide sequence of the light chain variable region of the human antibody C3022 (SEQ ID NO: 110 of the Sequence Listing).

FIG. 123 is a diagram showing the amino acid sequence of the light chain variable region of the human antibody C3022 (SEQ ID NO: 135 of the Sequence Listing).

FIG. 124 is a diagram showing the amino acid sequence of the heavy chain CDR1 of the human antibody C2037 (SEQ ID NO: 111 of the Sequence Listing).

FIG. 125 is a diagram showing the amino acid sequence of the heavy chain CDR2 of the human antibody C2037 (SEQ ID NO: 112 of the Sequence Listing).

FIG. 126 is a diagram showing the amino acid sequence of the heavy chain CDR3 of the human antibody C2037 (SEQ ID NO: 113 of the Sequence Listing).

FIG. 127 is a diagram showing the amino acid sequence of the light chain CDR1 of the human antibody C2037 (SEQ ID NO: 114 of the Sequence Listing).

FIG. 128 is a diagram showing the amino acid sequence of the light chain CDR2 of the human antibody C2037 (SEQ ID NO: 115 of the Sequence Listing).

FIG. 129 is a diagram showing the amino acid sequence of the light chain CDR3 of the human antibody C2037 (SEQ ID NO: 116 of the Sequence Listing).

FIG. 130 is a diagram showing the amino acid sequence of the heavy chain CDR1 of the human antibody C3048 (SEQ ID NO: 117 of the Sequence Listing).

FIG. 131 is a diagram showing the amino acid sequence of the heavy chain CDR2 of the human antibody C3048 (SEQ ID NO: 118 of the Sequence Listing).

FIG. 132 is a diagram showing the amino acid sequence of the heavy chain CDR3 of the human antibody C3048 (SEQ ID NO: 119 of the Sequence Listing).

FIG. 133 is a diagram showing the amino acid sequence of the light chain CDR1 of the human antibody C3048 (SEQ ID NO: 120 of the Sequence Listing).

FIG. 134 is a diagram showing the amino acid sequence of the light chain CDR2 of the human antibody C3048 (SEQ ID NO: 121 of the Sequence Listing).

FIG. 135 is a diagram showing the amino acid sequence of the light chain CDR3 of the human antibody C3048 (SEQ ID NO: 122 of the Sequence Listing).

FIG. 136 is a diagram showing the amino acid sequence of the heavy chain CDR1 of the human antibody C3015 (SEQ ID NO: 123 of the Sequence Listing).

FIG. 137 is a diagram showing the amino acid sequence of the heavy chain CDR2 of the human antibody C3015 (SEQ ID NO: 124 of the Sequence Listing).

FIG. 138 is a diagram showing the amino acid sequence of the heavy chain CDR3 of the human antibody C3015 (SEQ ID NO: 125 of the Sequence Listing).

FIG. 139 is a diagram showing the amino acid sequence of the light chain CDR1 of the human antibody C3015 (SEQ ID NO: 126 of the Sequence Listing).

FIG. 140 is a diagram showing the amino acid sequence of the light chain CDR2 of the human antibody C3015 (SEQ ID NO: 127 of the Sequence Listing).

FIG. 141 is a diagram showing the amino acid sequence of the light chain CDR3 of the human antibody C3015 (SEQ ID NO: 128 of the Sequence Listing).

FIG. 142 is a diagram showing the amino acid sequence of the heavy chain CDR1 of the human antibody C3022 (SEQ ID NO: 129 of the Sequence Listing).

FIG. 143 is a diagram showing the amino acid sequence of the heavy chain CDR2 of the human antibody C3022 (SEQ ID NO: 130 of the Sequence Listing).

FIG. 144 is a diagram showing the amino acid sequence of the heavy chain CDR3 of the human antibody C3022 (SEQ ID NO: 131 of the Sequence Listing).

FIG. 145 is a diagram showing the amino acid sequence of the light chain CDR1 of the human antibody C3022 (SEQ ID NO: 132 of the Sequence Listing).

FIG. 146 is a diagram showing the amino acid sequence of the light chain CDR2 of the human antibody C3022 (SEQ ID NO: 133 of the Sequence Listing).

FIG. 147 is a diagram showing the amino acid sequence of the light chain CDR3 of the human antibody C3022 (SEQ ID NO: 134 of the Sequence Listing).

FIG. 148 is a diagram showing the nucleotide sequence of the heavy chain of an IgG form of the human antibody C2037 (SEQ ID NO: 136 of the Sequence Listing).

FIG. 149 is a diagram showing the nucleotide sequence of the light chain of an IgG form of the human antibody C2037 (SEQ ID NO: 137 of the Sequence Listing).

FIG. 150 is a diagram showing the nucleotide sequence of the heavy chain of an IgG form of the human antibody C3048 (SEQ ID NO: 138 of the Sequence Listing).

FIG. 151 is a diagram showing the nucleotide sequence of the light chain of an IgG form of the human antibody C3048 (SEQ ID NO: 139 of the Sequence Listing).

FIG. 152 is a diagram showing the nucleotide sequence of the heavy chain of an IgG form of the human antibody C3015 (SEQ ID NO: 140 of the Sequence Listing).

FIG. 153 is a diagram showing the nucleotide sequence of the light chain of an IgG form of the human antibody C3015 (SEQ ID NO: 141 of the Sequence Listing).

FIG. 154 is a diagram showing the nucleotide sequence of the heavy chain of an IgG form of the human antibody C3022 (SEQ ID NO: 142 of the Sequence Listing).

FIG. 155 is a diagram showing the nucleotide sequence of the light chain of an IgG form of the human antibody C3022 (SEQ ID NO: 143 of the Sequence Listing).

FIG. 156 is a diagram showing the amino acid sequence of the heavy chain of the IgG form of the human antibody C2037 (signal sequence (amino acid positions 1 to 19), variable region (amino acid positions 20 to 134), constant region (amino acid positions 135 to 464)) (SEQ ID NO: 144 of the Sequence Listing).

FIG. 157 is a diagram showing the amino acid sequence of the light chain of the IgG form of the human antibody C2037 (signal sequence (amino acid positions 1 to 20), variable region (amino acid positions 21 to 130), constant region (amino acid positions 131 to 236)) (SEQ ID NO: 145 of the Sequence Listing).

FIG. 158 is a diagram showing the amino acid sequence of the heavy chain of the IgG form of the human antibody C3048 (signal sequence (amino acid positions 1 to 19), variable region (amino acid positions 20 to 142), constant region (amino acid positions 143 to 472)) (SEQ ID NO: 146 of the Sequence Listing).

FIG. 159 is a diagram showing the amino acid sequence of the light chain of the IgG form of the human antibody C3048 (signal sequence (amino acid positions 1 to 20), variable region (amino acid positions 21 to 130), constant region (amino acid positions 131 to 236)) (SEQ ID NO: 147 of the Sequence Listing).

FIG. 160 is a diagram showing the amino acid sequence of the heavy chain of the IgG form of the human antibody C3015 (signal sequence (amino acid positions 1 to 19), variable region (amino acid positions 20 to 140), constant region (amino acid positions 141 to 470)) (SEQ ID NO: 148 of the Sequence Listing).

FIG. 161 is a diagram showing the amino acid sequence of the light chain of the IgG form of the human antibody C3015 (signal sequence (amino acid positions 1 to 20), variable region (amino acid positions 21 to 126), constant region (amino acid positions 127 to 232)) (SEQ ID NO: 149 of the Sequence Listing).

FIG. 162 is a diagram showing the amino acid sequence of the heavy chain of the IgG form of the human antibody C3022 (signal sequence (amino acid positions 1 to 19), variable region (amino acid positions 20 to 134), constant region (amino acid positions 135 to 464)) (SEQ ID NO: 150 of the Sequence Listing).

FIG. 163 is a diagram showing the amino acid sequence of the light chain of the IgG form of the human antibody C3022 (signal sequence (amino acid positions 1 to 20), variable region (amino acid positions 21 to 130), constant region (amino acid positions 131 to 236)) (SEQ ID NO: 151 of the Sequence Listing).

FIG. 164 is a diagram showing results of testing the binding activity of human antibody scFv against the amino terminus of biotinylated human (A) or cynomolgus monkey (B) GPRC5D by ELISA. The vertical axis represents the luminescence intensity assayed by ELISA.

FIG. 165 is a diagram showing results of testing the binding activity of an IgG form of a human antibody against the amino terminus of biotinylated human or cynomolgus monkey GPRC5D by ELISA. The vertical axis represents the luminescence intensity assayed by ELISA.

FIG. 166 is a diagram showing results of testing the binding activity of human antibody scFv against a human GPRC5D-expressing cancer cell line using a flow cytometer (FACS). The vertical axis represents a relative value of the mean fluorescence intensity assayed by flow cytometry.

FIG. 167 is a diagram showing results of testing the binding activity of an IgG form of a human antibody against a human GPRC5D-expressing cancer cell line using a flow cytometer (FACS). The vertical axis represents a relative value of the mean fluorescence intensity assayed by flow cytometry.

FIG. 168 is a diagram showing a nucleotide sequence encoding the heavy chain variable region of a rat anti-CD3 antibody C3-147 (SEQ ID NO: 152).

FIG. 169 is a diagram showing a nucleotide sequence encoding the light chain variable region of the rat anti-CD3 antibody C3-147 (SEQ ID NO: 153).

FIG. 170 is a diagram showing a nucleotide sequence encoding C3E-7000(58-867) (signal sequence (nucleotide positions 1 to 57), scFv (nucleotide positions 58 to 783), FLAG-His tag (nucleotide positions 793 to 867)) (SEQ ID NO: 154).

FIG. 171 is a diagram showing the amino acid sequence of the heavy chain variable region of C3E-7034 (SEQ ID NO: 155).

FIG. 172 is a diagram showing the amino acid sequence of the light chain variable region of C3E-7034 (SEQ ID NO: 156).

FIG. 173 is a diagram showing a nucleotide sequence encoding C3E-7034(58-864) (signal sequence (nucleotide positions 1 to 57), scFv (nucleotide positions 61 to 786), FLAG-His tag (nucleotide positions 790 to 864)) (SEQ ID NO: 157).

FIG. 174 is a diagram showing the amino acid sequence of the light chain variable region of C3E-7035 (SEQ ID NO: 158).

FIG. 175 is a diagram showing a nucleotide sequence encoding C3E-7035(58-864) (signal sequence (nucleotide positions 1 to 57), scFv (nucleotide positions 61 to 786), FLAG-His tag (nucleotide positions 790 to 864)) (SEQ ID NO: 159).

FIG. 176 is a diagram showing the amino acid sequence of the light chain variable region of C3E-7036 (SEQ ID NO: 160).

FIG. 177 is a diagram showing a nucleotide sequence encoding C3E-7036(58-858) (signal sequence (nucleotide positions 1 to 57), scFv (nucleotide positions 61 to 780), FLAG-His tag (nucleotide positions 784 to 858)) (SEQ ID NO: 161).

FIG. 178 is a diagram showing a nucleotide sequence encoding an expression vector pC2037-C3E7034 (SEQ ID NO: 162).

FIG. 179 is a diagram showing a nucleotide sequence encoding ORF of an expression vector pC3048-C3E-7034 (SEQ ID NO: 163).

FIG. 180 is a diagram showing a nucleotide sequence encoding ORF of an expression vector pC3022-C3E-7034 (SEQ ID NO: 164).

FIG. 181 is a diagram showing a nucleotide sequence encoding ORF of an expression vector pC2037-C3E-7035 (SEQ ID NO: 165).

FIG. 182 is a diagram showing a nucleotide sequence encoding ORF of an expression vector pC3048-C3E-7035 (SEQ ID NO: 166).

FIG. 183 is a diagram showing a nucleotide sequence encoding ORF of an expression vector pC3022-C3E-7035 (SEQ ID NO: 167).

FIG. 184 is a diagram showing a nucleotide sequence encoding ORF of an expression vector pC2037-C3E-7036 (SEQ ID NO: 168).

FIG. 185 is a diagram showing a nucleotide sequence encoding ORF of an expression vector pC3048-C3E-7036 (SEQ ID NO: 169).

FIG. 186 is a diagram showing a nucleotide sequence encoding ORF of an expression vector pC3022-C3E-7036 (SEQ ID NO: 170).

FIG. 187 is a diagram showing the amino acid sequence of C2037-C3E-7034 (signal sequence (amino acid positions 1 to 19), C2037 (amino acid positions 21 to 260), C3E-7034 (amino acid positions 266 to 507)) (SEQ ID NO: 171).

FIG. 188 is a diagram showing the amino acid sequence of C3048-C3E-7034 (signal sequence (amino acid positions 1 to 19), C3048 (amino acid positions 21 to 268), C3E-7034 (amino acid positions 274 to 515)) (SEQ ID NO: 172).

FIG. 189 is a diagram showing the amino acid sequence of C3022-C3E-7034 (signal sequence (amino acid positions 1 to 19), C3022 (amino acid positions 21 to 260), C3E-7034 (amino acid positions 266 to 507)) (SEQ ID NO: 173).

FIG. 190 is a diagram showing the amino acid sequence of C2037-C3E-7035 (signal sequence (amino acid positions 1 to 19), C2037 (amino acid positions 21 to 260), C3E-7035 (amino acid positions 266 to 507)) (SEQ ID NO: 174).

FIG. 191 is a diagram showing the amino acid sequence of C3048-C3E-7035 (signal sequence (amino acid positions 1 to 19), C3048 (amino acid positions 21 to 268), C3E-7035 (amino acid positions 274 to 515)) (SEQ ID NO: 175).

FIG. 192 is a diagram showing the amino acid sequence of C3022-C3E-7035 (signal sequence (amino acid positions 1 to 19), C3022 (amino acid positions 21 to 260), C3E-7035 (amino acid positions 266 to 507)) (SEQ ID NO: 176).

FIG. 193 is a diagram showing the amino acid sequence of C2037-C3E-7036 (signal sequence (amino acid positions 1 to 19), C2037 (amino acid positions 21 to 260), C3E-7036 (amino acid positions 266 to 505)) (SEQ ID NO: 177).

FIG. 194 is a diagram showing the amino acid sequence of C3048-C3E-7036 (signal sequence (amino acid positions 1 to 19), C3048 (amino acid positions 21 to 268), C3E-7036 (amino acid positions 274 to 513)) (SEQ ID NO: 178).

FIG. 195 is a diagram showing the amino acid sequence of C3022-C3E-7036 (signal sequence (amino acid positions 1 to 19), C3022 (amino acid positions 21 to 260), C3E-7036 (amino acid positions 266 to 505)) (SEQ ID NO: 179).

FIG. 196 is a diagram showing results of testing the binding activity of an anti-GPRC5D-anti-CD3 bispecific molecule against cells expressing endogenous human GPRC5D (human lymphoma cell line A4/FuK cells) using a flow cytometer (FACS). The vertical axis represents a relative value of the mean fluorescence intensity assayed by flow cytometry.

FIG. 197 is a diagram showing results of testing the binding activity of the anti-GPRC5D-anti-CD3 bispecific molecule against cells expressing cynomolgus monkey GPRC5D using a flow cytometer (FACS). The vertical axis represents a relative value of the mean fluorescence intensity assayed by flow cytometry.

FIG. 198 is a diagram showing results of testing the binding activity of the anti-GPRC5D-anti-CD3 bispecific molecule against human CD3 (PBMC) using a flow cytometer (FACS). The vertical axis represents a relative value of the mean fluorescence intensity assayed by flow cytometry.

FIG. 199 is a diagram showing results of testing the binding activity of the anti-GPRC5D-anti-CD3 bispecific molecule against cynomolgus monkey CD3 (PBMC) using a flow cytometer (FACS). The vertical axis represents a relative value of the mean fluorescence intensity assayed by flow cytometry.

FIG. 200 is a diagram showing that the anti-GPRC5D-anti-CD3 bispecific molecule has cytotoxic activity against cells expressing endogenous human GPRC5D (human lymphoma cell line A4/FuK cells).

FIG. 201 is a diagram showing results of testing the binding activity of humanized 2B1 against cynomolgus monkey GPRC5D using a flow cytometer (FACS). The vertical axis represents a relative value of the mean fluorescence intensity assayed by flow cytometry.

FIG. 202 is a diagram showing results of testing the binding activity of humanized 7B4 against cynomolgus monkey GPRC5D using a flow cytometer (FACS). The vertical axis represents a relative value of the mean fluorescence intensity assayed by flow cytometry.

FIG. 203 is a diagram showing the amino acid sequence of C3E-7034 (amino acid positions 1 to 269). VH(2-119), VL(135-243), FLAG-His tag (244 to 269)) (SEQ ID NO: 180).

FIG. 204 is a diagram showing the amino acid sequence of C3E-7035(amino acid positions 1 to 269). VH(2-119), VL(135-243), FLAG-His tag (244 to 269)) (SEQ ID NO: 181).

FIG. 205 is a diagram showing the amino acid sequence of C3E-7036(amino acid positions 1 to 267). VH(2-119), VL(135-241), FLAG-His tag (242 to 267)) (SEQ ID NO: 182).

FIG. 206 is a diagram showing the amino acid sequence of the heavy chain CDR1 of C3E-7000 (SEQ ID NO: 183).

FIG. 207 is a diagram showing the amino acid sequence of the heavy chain CDR2 of C3E-7000 (SEQ ID NO: 184).

FIG. 208 is a diagram showing the amino acid sequence of the heavy chain CDR3 of C3E-7000 (SEQ ID NO: 185).

FIG. 209 is a diagram showing the amino acid sequence of the light chain CDR1 of C3E-7000 (SEQ ID NO: 186).

FIG. 210 is a diagram showing the amino acid sequence of the light chain CDR2 of C3E-7000 (SEQ ID NO: 187).

FIG. 211 is a diagram showing the amino acid sequence of the light chain CDR3 of C3E-7000 (SEQ ID NO: 188).

FIG. 212 is a diagram showing the amino acid sequence of human CD3c (SEQ ID NO: 189).

FIG. 213 is a diagram showing the nucleotide sequence of the heavy chain variable region of E1018 (SEQ ID NO: 190).

FIG. 214 is a diagram showing the amino acid sequence of the heavy chain variable region of E1018 (SEQ ID NO: 191).

FIG. 215 is a diagram showing the nucleotide sequence of the light chain variable region of E1018 (SEQ ID NO: 192).

FIG. 216 is a diagram showing the amino acid sequence of the light chain variable region of E1018 (SEQ ID NO: 193).

FIG. 217 is a diagram showing the nucleotide sequence of the heavy chain variable region of D1012 (SEQ ID NO: 194).

FIG. 218 is a diagram showing the amino acid sequence of the heavy chain variable region of D1012 (SEQ ID NO: 195).

FIG. 219 is a diagram showing the nucleotide sequence of the light chain variable region of D1012 (SEQ ID NO: 196).

FIG. 220 is a diagram showing the amino acid sequence of the light chain variable region of D1012 (SEQ ID NO: 197).

FIG. 221 is a diagram showing dissociation constants determined by assaying the binding activity of the anti-GPRC5D antibodies (C3022, E1018, C3048, and D1012) against human GPRC5D by SPR.

FIG. 222 is a diagram showing the nucleic sequence of h2B1_Fab_HC_1 (SEQ ID NO: 198).

FIG. 223 is a diagram showing the amino acid sequence of h2B1_Fab_HC_1 (signal sequence (amino acid positions 1 to 23), variable region (amino acid positions 24 to 146), constant region (amino acid positions 147 to 475)) (SEQ ID NO: 199).

FIG. 224 is a diagram showing the nucleic sequence of h2B1_Fab_HC_2 (SEQ ID NO: 200).

FIG. 225 is a diagram showing the amino acid sequence of h2B1_Fab_HC_2 (signal sequence (amino acid positions 1 to 23), variable region (amino acid positions 24 to 146), constant region (amino acid positions 147 to 475)) (SEQ ID NO: 201).

FIG. 226 is a diagram showing the nucleotide sequence of h2B1_Fab_LC_1 (SEQ ID NO: 202).

FIG. 227 is a diagram showing the amino acid sequence of h2B1_Fab_LC_1 (signal sequence (amino acid positions 1 to 23), variable region (amino acid positions 24 to 130), constant region (amino acid positions 131 to 237)) (SEQ ID NO: 203).

FIG. 228 is a diagram showing the nucleotide sequence of h2B1_Fab_LC_2 (SEQ ID NO: 204).

FIG. 229 is a diagram showing the amino acid sequence of h2B1_Fab_LC_2 (signal sequence (amino acid positions 1 to 23), variable region (amino acid positions 24 to 130), constant region (amino acid positions 131 to 237)) (SEQ ID NO: 205).

FIG. 230 is a diagram showing the nucleic sequence of C3E-7034_Fab_HC (SEQ ID NO: 206).

FIG. 231 is a diagram showing the amino acid sequence of C3E-7034_Fab_HC (signal sequence (amino acid positions 1 to 23), variable region (amino acid positions 25 to 142), constant region (amino acid positions 143 to 471)) (SEQ ID NO: 207).

FIG. 232 is a diagram showing the nucleotide sequence of C3E-7034_Fab_LC (SEQ ID NO: 208).

FIG. 233 is a diagram showing the amino acid sequence of C3E-7034_Fab_LC (signal sequence (amino acid positions 1 to 23), variable region (amino acid positions 24 to 132), constant region (amino acid positions 133 to 238)) (SEQ ID NO: 209).

FIG. 234 is a diagram showing the nucleic sequence of C3E-7036_Fab_HC (SEQ ID NO: 210).

FIG. 235 is a diagram showing the amino acid sequence of C3E-7036_Fab_HC (signal sequence (amino acid positions 1 to 23), variable region (amino acid positions 25 to 142), constant region (amino acid positions 143 to 471)) (SEQ ID NO: 211).

FIG. 236 is a diagram showing the nucleotide sequence of C3E-7036_Fab_LC (SEQ ID NO: 212).

FIG. 237 is a diagram showing the amino acid sequence of C3E-7036_Fab_LC (signal sequence (amino acid positions 1 to 23), variable region (amino acid positions 24 to 130), constant region (amino acid positions 131 to 236)) (SEQ ID NO: 213).

FIG. 238 is a diagram showing the nucleic sequence of h2B1_Fab_HC_3 (SEQ ID NO: 214).

FIG. 239 is a diagram showing the amino acid sequence of h2B1_Fab_HC_3 (signal sequence (amino acid positions 1 to 23), variable region (amino acid positions 24 to 146), constant region (amino acid positions 147 to 475)) (SEQ ID NO: 215).

FIG. 240 is a diagram showing the nucleotide sequence of h2B1_Fab_LC_3 (SEQ ID NO: 216).

FIG. 241 is a diagram showing the amino acid sequence of h2B1_Fab_LC_3 (signal sequence (amino acid positions 1 to 23), variable region (amino acid positions 24 to 130), constant region (amino acid positions 131 to 237)) (SEQ ID NO: 217).

FIG. 242 is a diagram showing the nucleotide sequence of C3E-7034_scFv_Fc (SEQ ID NO: 218).

FIG. 243 is a diagram showing the amino acid sequence of C3E-7034_scFv_Fc (signal sequence (amino acid positions 1 to 23), scFv (amino acid positions 24 to 266)) (SEQ ID NO: 219).

FIG. 2344 is a diagram showing the nucleotide sequence of C3E-7036_scFv_Fc (SEQ ID NO: 220).

FIG. 245 is a diagram showing the amino acid sequence of C3E-7036_scFv_Fc (signal sequence (amino acid positions 1 to 23), scFv (amino acid positions 24 to 264)) (SEQ ID NO: 221).

FIG. 246 is a diagram showing the nucleotide sequence of humanized 2B1_scFv_Fc (h2B1_scFv_Fc) (SEQ ID NO: 222).

FIG. 247 is a diagram showing the amino acid sequence of humanized 2B1_scFv_Fc (h2B1_scFv_Fc) (signal sequence (amino acid positions 1 to 23), scFv (amino acid positions 24 to 271)) (SEQ ID NO: 223).

FIG. 248 is a diagram showing results of testing the binding activity of a Fc-containing anti-GPRC5D-anti-CD3 bispecific molecule against cells expressing endogenous human GPRC5D by flow cytometry. The vertical axis represents the mean fluorescence intensity assayed by flow cytometry.

A, B, and C are diagrams showing results of testing the binding activity of bispecific molecule of FSA type, Hybrid type and Dual type respectively.

FIG. 249 is a diagram showing results of testing the binding activity of a Fc-containing anti-GPRC5D-anti-CD3 bispecific molecule against cells expressing cynomolgus monkey GPRC5D by flow cytometry. The vertical axis represents the mean fluorescence intensity assayed by flow cytometry.

A, B, and C are diagrams showing results of testing the binding activity of bispecific molecule of FSA type, Hybrid type and Dual type respectively.

FIG. 250 is a diagram showing results of testing the binding activity of the Fc-containing anti-GPRC5D-anti-CD3 bispecific molecule against cells expressing human CD3 by flow cytometry. The vertical axis represents the mean fluorescence intensity assayed by flow cytometry.

A, B, and C are diagrams showing results of testing the binding activity of bispecific molecule of FSA type, Hybrid type and Dual type respectively.

FIG. 251 is a diagram showing results of testing the binding activity of the Fc-containing anti-GPRC5D-anti-CD3 bispecific molecule against cells expressing cynomolgus monkey CD3 by flow cytometry. The vertical axis represents the mean fluorescence intensity assayed by flow cytometry.

A, B, and C are diagrams showing results of testing the binding activity of bispecific molecule of FSA type, Hybrid type and Dual type respectively.

FIG. 252 is a diagram showing the cytotoxic activity of the Fc-containing anti-GPRC5D-anti-CD3 bispecific molecule.

A and B are diagrams showing results of testing the binding activity of bispecific molecule of FSA type and Hybrid type respectively.

FIG. 252 is a diagram showing the cytotoxic activity of the Fc-containing anti-GPRC5D-anti-CD3 bispecific molecule.

C is a diagrams showing results of testing the binding activity of bispecific molecule of Dual type.

FIG. 253 is a diagram showing the anti-tumor activity of the Fc-containing hybrid-type anti-GPRC5D-anti-CD3 bispecific molecule in tumor/PBMC co-grafting model.

FIG. 254 is a diagram showing the anti-tumor activity of Fc-containing hybrid-type anti-GPRC5D-anti-CD3 bispecific molecule in human PBMC-transferred model.

FIG. 255 is a diagram showing the nucleotide sequence of C3E-8015 (SEQ ID NO: 224).

FIG. 256 is a diagram showing the amino acid sequence of C3E-8015 (signal sequence (amino acid positions 1 to 23), scFv (amino acid positions 24 to 264)) (SEQ ID NO: 225).

FIG. 257 is a diagram showing the nucleotide sequence of C3E-8017 (SEQ ID NO: 226).

FIG. 258 is a diagram showing the amino acid sequence of C3E-8017 (signal sequence (amino acid positions 1 to 23), scFv (amino acid positions 24 to 266)) (SEQ ID NO: 227).

FIG. 259 is a diagram showing the nucleotide sequence of C3E-8018 (SEQ ID NO: 228).

FIG. 260 is a diagram showing the amino acid sequence of C3E-8018 (signal sequence (amino acid positions 1 to 23), scFv (amino acid positions 24 to 266)) (SEQ ID NO: 229).

FIG. 261 is a diagram showing the nucleotide sequence of C3E-8025 (SEQ ID NO: 230).

FIG. 262 is a diagram showing the amino acid sequence of C3E-8025 (signal sequence (amino acid positions 1 to 23), scFv (amino acid positions 24 to 264)) (SEQ ID NO: 231).

FIG. 263 is a diagram showing the nucleotide sequence of C3E-8027 (SEQ ID NO: 232).

FIG. 264 is a diagram showing the amino acid sequence of C3E-8027 (signal sequence (amino acid positions 1 to 23), scFv (amino acid positions 24 to 266)) (SEQ ID NO: 233).

FIG. 265 is a diagram showing the nucleotide sequence of C3E-8028 (SEQ ID NO: 234).

FIG. 266 is a diagram showing the amino acid sequence of C3E-8028 (signal sequence (amino acid positions 1 to 23), scFv (amino acid positions 24 to 266)) (SEQ ID NO: 235).

FIG. 267 is a diagram showing the nucleotide sequence of h2B1_Fab_HC_4 (SEQ ID NO: 236).

FIG. 268 is a diagram showing the amino acid sequence of h2B1_Fab_HC_4 (signal sequence (amino acid positions 1 to 23), variable region (amino acid positions 24 to 146), constant region (amino acid positions 147 to 476)) (SEQ ID NO: 237).

FIG. 269 is diagrams showing results of testing the binding activity of CDR-modified hybrid-type and C-terminally Lys-added CDR-modified hybrid-type anti-GPRC5D-anti-CD3 bispecific molecule against cells expressing endogenous human GPRC5D by flow cytometry. The vertical axis represents the mean fluorescence intensity assayed by flow cytometry. A, B, and C are diagrams showing results of testing the binding activity of (C5D-0004 and C5D-0014), (C5D-0005 and C5D-0015) and (C5D-0006 and C5D-0016) respectively.

FIG. 270 is diagrams showing results of testing the binding activity of CDR-modified hybrid-type and C-terminally Lys-added CDR-modified hybrid-type anti-GPRC5D-anti-CD3 bispecific molecule against cells expressing cynomolgus monkey GPRC5D by flow cytometry. The vertical axis represents the mean fluorescence intensity assayed by flow cytometry. A, B, and C are diagrams showing results of testing the binding activity of (C5D-0004 and C5D-0014), (C5D-0005 and C5D-0015) and (C5D-0006 and C5D-0016) respectively.

FIG. 271 is diagrams showing results of testing the binding activity of CDR-modified hybrid-type and C-terminally Lys-added CDR-modified hybrid-type anti-GPRC5D-anti-CD3 bispecific molecule against cells expressing human CD3 by flow cytometry. The vertical axis represents the mean fluorescence intensity assayed by flow cytometry.

A, B, and C are diagrams showing results of testing the binding activity of (C5D-0004 and C5D-0014), (C5D-0005 and C5D-0015) and (C5D-0006 and C5D-0016) respectively.

FIG. 272 is a diagram showing results of testing the binding activity of CDR-modified hybrid-type and C-terminally Lys-added CDR-modified hybrid-type anti-GPRC5D-anti-CD3 bispecific molecule against cells expressing cynomolgus monkey CD3 by flow cytometry. The vertical axis represents the mean fluorescence intensity assayed by flow cytometry. A, B, and C are diagrams showing results of testing the binding activity of (C5D-0004 and C5D-0014), (C5D-0005 and C5D-0015) and (C5D-0006 and C5D-0016) respectively.

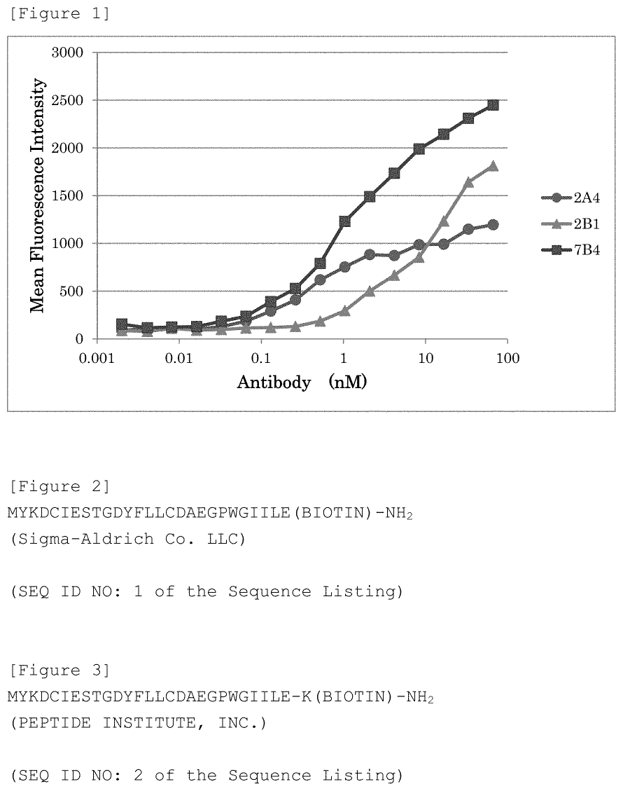

FIG. 273-1 is diagrams showing the cytotoxic activity of CDR-modified hybrid-type and C-terminally Lys-added CDR-modified hybrid-type anti-GPRC5D-anti-CD3 bispecific molecule.

A and B are diagrams showing the cytotoxic activity of C5D-0004 and C5D-0014 respectively.

FIG. 273-2 is diagrams showing the cytotoxic activity of CDR-modified hybrid-type and C-terminally Lys-added CDR-modified hybrid-type anti-GPRC5D-anti-CD3 bispecific molecule. C and D are diagrams showing the cytotoxic activity of C5D-0005 and C5D-0015 respectively.

FIG. 273-3 is diagrams showing the cytotoxic activity of CDR-modified hybrid-type and C-terminally Lys-added CDR-modified hybrid-type anti-GPRC5D-anti-CD3 bispecific molecule.

E and F are diagrams showing the cytotoxic activity of C5D-0006 and C5D-0016 respectively.

FIG. 274 is a diagram showing the anti-tumor activity of the CDR-modified Hybrid type anti-GPRC5D-anti-CD3 bispecific molecule in tumor/PBMC co-grafting model.

FIG. 275 are diagrams showing the tumor regression activity of the CDR-modified hybrid-type (A), and C-terminally Lys-added CDR-modified hybrid-type (B) anti-GPRC5D-anti-CD3 bispecific molecule in established tumor model in human PBMC reconstituted mice.

A and B are diagrams showing the tumor regression activity of C5D-0004 and C5D-0014 respectively.

FIG. 276 shows the amino acid sequence of the CDR-modified heavy chain CDR2 (SEQ ID NO: 238).

FIG. 277 shows the amino acid sequence of the CDR-modified light chain CDR2 (SEQ ID NO: 239).

FIG. 278 shows the amino acid sequence of the heavy chain variable region of a CDR-modified C3E-7034 (SEQ ID NO: 240).

FIG. 279 shows the amino acid sequence of the light chain variable region of CDR-modified C3E-7034 (SEQ ID NO: 241).

FIG. 280 shows the amino acid sequence of the light chain variable region of CDR-modified C3E-7035 (SEQ ID NO: 242).

FIG. 281 shows the amino acid sequence of the light chain variable region of CDR-modified C3E-7036 (SEQ ID NO: 243).

FIG. 282 shows the amino acid sequence of C3E-7078(1-269). VH(2-119), VL(135-243), FLAG-His tag(244-269) (SEQ ID NO: 244).

FIG. 283 shows the amino acid sequence of C3E-7085(1-267). VH(2-119), VL(135-241), FLAG-His tag(242-267) (SEQ ID NO: 245).

FIG. 284 shows the amino acid sequence of C3E-7086(1-269). VH(2-119), VL(135-243), FLAG-His tag (244-269) (SEQ ID NO: 246).

FIG. 285 shows the amino acid sequence of C3E-7087(1-269). VH(2-119), VL(135-243), FLAG-His tag(244-269) (SEQ ID NO: 247).

FIG. 286 shows the amino acid sequence of C3E-7088(1-269). VH(2-119), VL(135-243), FLAG-His tag(244-269) (SEQ ID NO: 248).

FIG. 287 shows the amino acid sequence of C3E-7089(1-269). VH(2-119), VL(135-243), FLAG-His tag(244-269) (SEQ ID NO: 249).

FIG. 288 shows the amino acid sequence of C3E-7090(1-269). VH(2-119), VL(135-243), FLAG-His tag(244-269) (SEQ ID NO: 250).

FIG. 289 shows the amino acid sequence of C3E-7091(1-269). VH(2-119), VL(135-243), FLAG-His tag(244-269) (SEQ ID NO: 251).

FIG. 290 shows the amino acid sequence of C3E-7092(1-269). VH(2-119), VL(135-243), FLAG-His tag(244-269) (SEQ ID NO: 252).

FIG. 291 shows the amino acid sequence of C3E-7093(1-269). VH(2-119), VL(135-243), FLAG-His tag(244-269) (SEQ ID NO: 253).

FIG. 292 shows the amino acid sequence of C3E-7094(1-269). VH(2-119), VL(135-243), FLAG-His tag(244-269) (SEQ ID NO: 254).

FIG. 293 shows the amino acid sequence of C3E-7095(1-269). VH(2-119), VL(135-243), FLAG-His tag(244-269) (SEQ ID NO: 255).

### DESCRIPTION OF EMBODIMENTS

### 1. Definitions

In the present invention, the term “gene” means a nucleotide comprising a nucleotide sequence encoding the amino acids of a protein, or its complementary strand. The “gene” is meant to include, for example, a polynucleotide, an oligonucleotide, DNA, mRNA, cDNA, and cRNA as the polynucleotide comprising a nucleotide sequence encoding the amino acids of a protein, or its complementary strand. Such a gene is a single-stranded, double-stranded, or triple or more stranded nucleotide. The “gene” is also meant to include an association of DNA and RNA strands, a mixture of ribonucleotides (RNAs) and deoxyribonucleotides (DNAs) on one nucleotide strand, and a double-stranded or triple or more stranded nucleotide comprising such a nucleotide strand. In the present invention, “a base sequence” has the same meaning as “a nucleotide sequence.”

In the present invention, the term “polynucleotide” has the same meaning as a “nucleic acid” and a “nucleic acid molecule” and is also meant to include, for example, DNA, RNA, a probe, an oligonucleotide, and a primer. Such a polynucleotide is a single-stranded, double-stranded, or triple or more stranded polynucleotide. The “polynucleotide” is also meant to include an association of DNA and RNA strands, a mixture of ribonucleotides (RNAs) and deoxyribonucleotides (DNAs) on one polynucleotide strand, and an association of two strands or three or more strands comprising such a polynucleotide strand.

In the present invention, the terms “polypeptide”, “peptide”, and “protein” have the same meaning.

In the present invention, the term “antigen” has the same meaning as “immunogen”.

In the present invention, the term “cell” also includes, for example, various cells derived from individual animals, subcultured cells, primary cultured cells, cell lines, recombinant cells, and microbial cells.

In the present invention, the term “antibody” has the same meaning as an immunoglobulin. However, the “antibody” used for the anti-GPRC5D antibody of the present invention or the anti-CD3 antibody of the present invention means an immunoglobulin having constant and variable regions. The antibody is not particularly limited and may be a natural immunoglobulin or may be an immunoglobulin produced by partial or complete synthesis. The anti-GPRC5D antibody and/or the anti-CD3 antibody of the present invention is included in the “molecule” described later.

The basic structure of a quaternary antibody is constituted by two identical light (L) chains and two identical heavy (H) chains. Each light chain is linked to the heavy chain by one covalent disulfide bond. The two heavy chains are linked to each other by one or more disulfide bond(s) according to the isotypes of the heavy chains. Each of the light and heavy chains has regularly spaced intrachain disulfide bonds. Each of the heavy and light chains contains a constant region which exhibits a very high degree of amino acid sequence similarity and a variable region which exhibits a low degree of amino acid sequence similarity. The light chain has a variable region (VL) at the amino terminus followed by a constant region (CL). The heavy chain has a variable region (VH) at the amino terminus followed by three constant regions (CH1, CH2, and CH3). VL and VH are paired with each other, and CL is aligned with the first constant region (CH1) of the heavy chain. The pair of VL and VH forms a single antigen-binding site.

Fab is composed of heavy chain CH1 followed by VH, and light chain CL followed by VL. VH and VL each contain complementarity determining regions (CDRs).

Fc is constituted by the carboxyl-terminal regions of the heavy chain constant regions and is a dimer containing CH2 and CH3. The Fc of the present invention may be Fc having a natural sequence (natural Fc) or may be a mutated form of Fc containing a mutation in the natural sequence (mutated Fc).

Examples of the “mutated Fc” can include, but are not limited to a modified Fc region comprised in a heteromultimer (including a heterodimeric region) with stability increased disclosed in WO2013/063702; Fc comprising the CH3 domain of an immunoglobulin derived from an I antibody which has “protuberance” and “cavity” and included in heteromultimer disclosed in WO96/27011; Fc comprising CH3 domain included in heterodimer which is electrostatically favorable achieved by replacing one or more amino acid residues with a charged amino acid disclosed in WO2009/089004; heterodimeric regions comprised in heterodimer which is steric variant and/or pi (Isoelectric point) variant disclosed in WO2014/110601; heterodimeric. Fc comprising CH3 domain that eradicates reduces binding to Protein A disclosed in WO2010/151792.

The variable region is composed of regions, called hypervariable regions (HVRs), having extreme variability, and relatively invariable regions, called framework regions (FRs), interrupted by the hypervariable regions. The natural heavy and light chain variable regions each contain four FRs connected by three hypervariable regions. The hypervariable regions of each chain are kept in close proximity together with the hypervariable regions of another chain by FRs and contribute to the formation of an antigen-binding site in the antibody.

The heavy and light chains of an antibody molecule are known to each have three complementarity determining regions (CDRs). The complementarity determining regions are also called hypervariable domains. These regions are located in the variable regions of the antibody heavy and light chains. These sites have a particularly highly variable primary structure and are usually separated at three positions on the respective primary structures of heavy and light chain polypeptide strands. In the present invention, the complementarity determining regions of the antibody are referred to as heavy chain CDR1 (CDRH1), heavy chain CDR2 (CDRH2), and heavy chain CDR3 (CDRH3) from the amino terminus of the heavy chain amino acid sequence for the complementarity determining regions of the heavy chain and as light chain CDR1 (CDRL1), light chain CDR2 (CDRL2), and light chain CDR3 (CDRL3) from the amino terminus of the light chain amino acid sequence for the complementarity determining regions of the light chain. These sites are proximal to each other on the three-dimensional structure and determine specificity for the antigen to be bound.

In the present invention, the positions and lengths of CDRs were determined according to the definition of IMGT (Developmental and Comparative Immunology 27 (2003) 55-77).

Framework regions (FRs) are variable regions except for the CDR residues. Each variable region generally has four FRs: FR1, FR2, FR3, and FR4. Heavy and light chain FRs are referred to as FRH1, FRH2, FRH3, and FRH4, and FRL1, FRL2, FRL3, and FRL4, respectively.

The CDRs and the FRs contained in the heavy and light chains are positioned as FRH1-CDRH1-FRH2-CDRH2-FRH3-CDRH3-FRH4 and FRL1-CDRL1-FRL2-CDRL2-FRL3-CDRL3-FRL4 in this order from the amino terminus toward the carboxyl terminus.

The CDRs and the FRs contained in the heavy and light chains are positioned as FRH1-CDRH1-FRH2-CDRH2-FRH3-CDRH3-FRH4 and FRL1-CDRL1-FRL2-CDRL2-FRL3-CDRL3-FRL4 in this order from the amino terminus toward the carboxyl terminus.

The positions of CDRs and FRs can also be determined according to various definitions well known in the art, for example, the definition of IMGT as well as Kabat, Chothia, AbM, contact, etc.

In the present invention, the term “antigen-binding fragment of the antibody” means a partial antibody fragment that is constituted by heavy and light chain variable regions and has binding activity against the antigen. Examples of the “antigen-binding fragment of the antibody” can include, but are not limited to, antigen-binding fragments such as Fab, F(ab′)₂, scFv, Fab′, Fv, and single-domain antibody (sdAb). Such an antigen-binding fragment of the antibody may be obtained by treating a full-length molecule of the antibody protein with an enzyme such as papain or pepsin or may be a recombinant protein produced in an appropriate host cell using a recombinant gene.

In the present invention, the “site” to which an antibody binds, i.e., the “site” recognized by an antibody, means a partial peptide or partial conformation on an antigen bound or recognized by the antibody.

In the present invention, such a site is also referred to as an epitope or an antibody binding site.

In the present invention, the term “antibody mutant” means a polypeptide that has an amino acid sequence derived from the amino acid sequence of the original antibody by the substitution, deletion, and/or addition (the addition includes insertion) (hereinafter, collectively referred to as a “mutation”) of amino acid(s) and binds to the antigen. The number of mutated amino acids in such an antibody mutant is 1 to 2, 3, 4, 5, 6, 7, 8, 9, 10, 12, 15, 20, 25, 30, 40, or 50. Such an antibody mutant is also encompassed by the “antibody” of the present invention.

In the present invention, the term “several” in “1 to several” refers to 2 to 10.

In the present specification, the term “molecule” is a molecule comprising the aforementioned antibody or antigen-binding fragment of the antibody and may be a multispecific molecule formed by antibodies or a plurality of antigen-binding fragments derived therefrom.

In the present specification, the term “molecule which is multispecific” has the same meaning as a “multispecific molecule”. Such a multispecific molecule is not particularly limited as long as the molecule is capable of binding to a plurality of epitopes different from each other on one molecule, and/or epitopes different from each other on two or more molecules. The molecule which is multispecific also includes an antibody comprising heavy chain variable (VH) and light chain variable (VL) regions. Examples of such a multispecific molecule include, but are not limited to, a full-length antibody molecule having two or more types of heavy chains and two or more types of light chains, i.e., an IgG-type multispecific molecule, and a molecule consisting of two or more types of antigen-binding fragments having VLs and VHs, i.e., a molecule derived by a combination of Fab, Fab′, Fv, scFv, sdAb, etc. (i.e., tandem scFv, diabodies, single chain diabodies, and triabodies). In addition, a molecule formed by genetically or chemically linking a protein having antigen binding activity without having an immunoglobulin skeleton, to an antigen-binding fragment is also included in the multispecific molecule.

Examples of activities or properties exerted by the anti-CD3 antibody of the present invention or antigen-binding fragment of the antibody, or the multispecific molecule of the present invention can include biological activities and physicochemical properties and can specifically include various biological activities, binding activity against an antigen or an epitope, stability during production or storage, and thermal stability.

In the present invention, the phrase “hybridizing under stringent conditions” means hybridization under conditions involving hybridization at 65° C. in a solution containing 5×SSC, followed by washing at 65° C. for 20 minutes in an aqueous solution containing 2×SSC-0.1% SDS, at 65° C. for 20 minutes in an aqueous solution containing 0.5×SSC-0.1% SDS, and at 65° C. for 20 minutes in an aqueous solution containing 0.2×SSC-0.1% SDS, or hybridization under conditions equivalent thereto. SSC means an aqueous solution of 150 mM NaCl-15 mM sodium citrate, and n×SSC means SSC with an n-fold concentration.

In the present invention, the term “cytotoxicity” refers to some pathological change brought about to cells in one way or another and means not only direct trauma but every structural or functional damage to cells, including DNA cleavage, formation of base dimers, chromosomal break, damage on mitotic apparatus, and reduction in the activities of various enzymes.

In the present invention, the term “cytotoxic activity” means activity that causes the cytotoxicity mentioned above.

In the present invention, the term “antibody dependent cellular cytotoxicity activity”, also called “ADCC activity”, means the effect or activity of damaging target cells such as tumor cells by NK cells via antibodies.

In the present invention, the term “cytotoxic activity by the redirection of T cells” means that the cytotoxicity is caused via a multispecific molecule comprising an anti-target antigen antibody such as an anti-tumor antigen and the anti-CD3 antibody. Preferably, the term means that the anti-tumor antigen antibody binds to target tumor cells while the anti-CD3 antibody binds to T cells so that the target tumor cells and the T cells come close to each other to induce T cell activation-mediated cytotoxicity. The molecule can be contained in a pharmaceutical composition.

In the present invention, the terms “naturally occurring amino acid” and “naturally occurring amino acid residue” mean Ala (A), Arg (R), Asn (N), Asp (D), Cys (C), Gln (Q), Glu (E), Gly (G), His (H), Ile (I), Leu (L), Lys (K), Met (M), Phe (F), Pro (P), Ser (S), Thr (T), Trp (W), Tyr (Y), and Val (V) and their residues, and are also referred to as a “natural amino acid” or a “natural amino acid residue”.

### 2. Antigenic Protein

### 2-1. GPRC5D Antigen

In the present invention, the term “GPRC5D” has the same meaning as a GPRC5D protein.

GPRC5D is classified into group 5 of the G-protein coupled receptor family C and is one of the human GPCR proteins newly found by the homology search of the EST database using the amino acid sequences of a series of human GPCRs (Non Patent Literature 1). This protein has been registered under GenBank deposition Nos: AF209923, NM_018654, and NP_0611124. However, the physiological functions or physiological ligand of GPRC5D, the subtype of G protein (a subunit) to be coupled therewith, etc. have not yet been revealed.

### 2-2. CD3 Antigen

In the present invention, the term “CD3” has the same meaning as a CD3 protein.

CD3 is expressed, as a portion of a multimolecular T cell receptor complex, on T cells and is a complex of 5 types of polypeptides (γ, δ, ε, ζ, and η chains; molecular weights: 25000 to 28000, 21000, 20000, 16000, and 22000, respectively).

Examples of the CD3 complex include γ, δ, ε, ζ, and η chains. These are also called subunits. Anti-CD3 antibodies bind to T cells to induce T cell activation-mediated cytotoxicity. Many anti-CD3 antibodies bind to CD3ε.

The nucleotide sequence of a cDNA encoding human CD3ε is registered in GenBank under Accession No. NM_000733.3. The nucleotide sequence of a cDNA encoding cynomolgus monkey CD3 is registered in GenBank under Accession No. NM_001283615.1. The amino acid sequence of human CD3ε is described in SEQ ID NO: 189 of the Sequence Listing.

### 2-3. Preparation of Antigenic Protein

Each aforementioned antigenic protein (GPRC5D or CD3 (hereinafter, GPRC5D and CD3 are also collectively referred to as the antigenic protein) used in the present invention can be prepared by purification or isolation from animal tissues (including body fluids), cells derived from the tissues, or cultures of the cells, gene recombination, in vitro translation, chemical synthesis, etc.

The cDNA of the antigenic protein can be obtained by, for example, a so-called PCR method which performs polymerase chain reaction (hereinafter, referred to as “PCR”) (Saiki, R. K., et al., Science (1988) 239, 487-489) using a cDNA library from organs expressing the mRNA of the antigenic protein as a template and using primers capable of specifically amplifying the cDNA of the antigenic protein.

A polynucleotide hybridizing under stringent conditions to a polynucleotide consisting of a nucleotide sequence complementary to the nucleotide sequence encoding the antigenic protein expressed in a human or a rat, and encoding a protein having biological activities equivalent to the antigenic protein is also included in the cDNA of the antigenic protein.

In addition, splicing variants transcribed from the gene loci of the antigenic protein expressed in a human or a rat, or polynucleotides hybridizing thereto under stringent conditions and encoding a protein having biological activities equivalent to the antigenic protein are also included in the cDNA of the antigenic protein.

A nucleotide sequence encoding a protein that consists of an amino acid sequence derived from the amino acid sequence of the human or rat antigenic protein or the amino acid sequence thereof except for a signal sequence by the substitution, deletion, or addition of 1 to several amino acid(s) and has biological activities equivalent to the antigenic protein is also included in the nucleotide sequence of the antigenic protein gene.

A protein that consists of an amino acid sequence encoded by a splicing variant transcribed from the gene loci of the human or rat antigenic protein or an amino acid sequence derived from the amino acid sequence by the substitution, deletion, or addition of 1 to several amino acid(s) and has biological activities equivalent to the antigenic protein is also included in the antigenic protein.

### 2-4 Binding Specificity for Antigenic Protein

The anti-GPRC5D antibody of the present invention or antigen-binding fragment thereof, etc. recognizes human GPRC5D. In other words, the anti-GPRC5D antibody of the present invention or antigen-binding fragment thereof, etc. binds to a GPRC5D antigen and preferably binds to human GPRC5D and monkey GPRC5D, more preferably human GPRC5D and cynomolgus monkey GPRC5D. The humanized anti-GPRC5D antibody h2B1 antibody and the human antibody C3048 of the present invention described later further bind to cynomolgus monkey GPRC5D in addition to human GPRC5D.

The anti-CD3 antibody or antigen-binding fragment thereof, etc. contained in the multispecific molecule of the present invention recognizes a CD3 antigen, i.e., binds thereto. The anti-CD3 antibody or antigen-binding fragment thereof, etc. contained in the multispecific molecule of the present invention preferably binds to human CD3, monkey CD3, and the like and more preferably binds to human CD3 and cynomolgus monkey CD3.

The antibody that binds to the human and cynomolgus monkey antigenic proteins or antigen-binding fragment thereof can be subjected to various tests on efficacy or safety using primates, particularly, cynomolgus monkeys, useful for the nonclinical development (preclinical development) of pharmaceutical products, and is thus preferred.

Meanwhile, preferably, the anti-GPRC5D antibody of the present invention does not bind to mouse and/or rat GPRC5D. Therefore, for example, various assays or immunohistochemical tests using human GPRC5D gene-transfected mouse cells, tissues, or individuals (including transgenic animals, knockout animals, and knock-in animals) and the antibody or the multispecific molecule of the present invention, etc. can be carried out without being influenced by GPRC5D of the host mice and/or rats. Thus, the antibody or the multispecific molecule of the present invention, etc. is preferred for the research and nonclinical development, using mice, of drugs, animal drugs, or diagnostic drugs, etc., comprising the antibody or the multispecific molecule of the present invention, etc.

Likewise, preferably, the anti-CD3 antibody contained in the multispecific molecule of the present invention does not bind to mouse and/or rat CD3. Therefore, for example, various assays or immunohistochemical tests using human CD3 gene-transfected mouse cells, tissues, or individuals (including transgenic animals, knockout animals, and knock-in animals) and the antibody or the multispecific molecule of the present invention, etc. can be carried out without being influenced by CD3 of the host mice and/or rats. Thus, the antibody or the multispecific molecule of the present invention, etc. is preferred for the research and nonclinical development, using mice, of drugs, animal drugs, or diagnostic drugs, etc., comprising the antibody or the multispecific molecule of the present invention, etc.

In the present invention, the term “recognition”, i.e., “binding”, means binding which is not non-specific adsorption. Examples of criteria for determination of whether recognition is achieved or not, i.e., binding is achieved or not can include a dissociation constant (hereinafter, referred to as “K_{D}”). Preferably, the antibody, etc. of the present invention has a K_{D} value of 1×10⁻³ M or lower, 5×10⁻⁶ M or lower, 2×10⁻⁶ M or lower, or 1×10⁻⁶ M or lower for CD3.

In the present invention, the binding of the antibody to the antigen can be assayed or determined using a biomolecular interaction analysis system (e.g., SPR or BLI), ELISA, or RIA, or the like. The binding of the antibody to the antigen expressed on cell surface can be assayed by flow cytometry or the like.

The SPR (surface plasmon resonance analysis) method is used as an analysis approach of determining a dissociation constant (K_{D} value), etc. as an index for affinity by measuring an association rate constant (Ka value) and a dissociation rate constant (Kd value) by kinetic analysis. Examples of equipment used in the SPR analysis can include BIAcore™ (manufactured by GE Healthcare Bio-Sciences Corp.), ProteOn™ (manufactured by Bio-Rad Laboratories, Inc.), SPR-Navi™ (manufactured by BioNavis Oy Ltd.), Spreeta™ (manufactured by Texas Instruments Inc.), SPRi-Plex II™ (manufactured by Horiba, Ltd.), and Autolab SPR™ (manufactured by Metrohm Japan Ltd.).

BLI (biolayer interferometry) is a method which involves measuring biomolecular interaction using biolayer interference. Examples of equipment used in BLI interaction analysis include Octet system (manufactured by Pall ForteBio Corp.).

The ELISA is a method which involves capturing an antigen or an antibody of interest contained in a sample solution using a specific antibody or antigen, while detecting and quantifying the antigen or antibody of interest through the use of enzymatic reaction. An enzyme-labeled antigen or antibody is incorporated into the reaction system, and the enzyme activity is detected. For the enzyme activity detection, a substrate whose absorption spectrum is changed by the reaction is used, and the absorption spectrum is digitized by absorbance measurement.

The Cell-ELISA is a method which involves capturing an analyte on cell surface on a cell basis, while detecting and quantifying the analyte through the use of enzymatic reaction.

The RIA (radio immunoassay) can quantify an antibody by labeling the antibody with a radioactive material and measuring radioactivity from the antibody.

The flow cytometry is an approach of optically analyzing individual cells by dispersing cells into a fluid and flowing a thin stream of the fluid. A fluorescent dye-labeled antibody binds to a cell surface antigen through antigen-antibody reaction, and fluorescence intensity from the labeled antibody bound with cells is measured to quantify the antigen binding activity of the antibody.

### 3. Anti-GPRC5D Antibody

### 3-1. Type of Anti-GPRC5D Antibody

The anti-GPRC5D antibody of the present invention may be either monoclonal or polyclonal antibodies. Examples of the polyclonal antibody can include a mixture of plural types of antibodies differing in a portion or the whole of CDR sets. Examples of the monoclonal antibody can include non-human animal-derived antibodies (non-human animal antibodies), human antibodies, chimeric antibodies, and humanized antibodies, etc.

Examples of the non-human animal antibody can include antibodies derived from vertebrates such as mammals and birds. Examples of the mammal-derived antibody can include rodent-derived antibodies such as mouse antibodies and rat antibodies. Examples of the bird-derived antibody can include chicken antibodies. Examples of the anti-human GPRC5D rat monoclonal antibody can include 2A4, 2B1 and 7B4 (Example 1) of the present invention.

The amino acid sequence of the heavy chain variable region of 2A4 is shown in SEQ ID NO: 5 of the Sequence Listing. The amino acid sequence of the heavy chain variable region of 2B1 is shown in SEQ ID NO: 7 of the Sequence Listing. The amino acid sequence of the heavy chain variable region of 7B4 is shown in SEQ ID NO: 9 of the Sequence Listing.

The amino acid sequence of the light chain variable region of 2A4 is shown in SEQ ID NO: 12 of the Sequence Listing. The amino acid sequence of the light chain variable region of 2B1 is shown in SEQ ID NO: 14 of the Sequence Listing. The amino acid sequence of the light chain variable region of 7B4 is shown in SEQ ID NO: 16 of the Sequence Listing.

2A4, 2B1, and 7B4 have ADCC activity (Example 2).

Examples of the chimeric antibody can include an antibody comprising non-human animal antibody-derived variable regions bound with human antibody (human immunoglobulin) constant regions.

Examples of the chimeric antibody derived from the rat anti-human GPRC5D antibody 2A4 can include an antibody consisting of a light chain comprising a light chain variable region consisting of amino acid residues 21 to 127 of SEQ ID NO: 22 and a heavy chain comprising a heavy chain variable region consisting of amino acid residues 20 to 141 of SEQ ID NO: 26. One example of such a 2A4-derived chimeric antibody can include an antibody consisting of a light chain consisting of amino acid residues 21 to 234 of SEQ ID NO: 22 and a heavy chain consisting of amino acid residues 20 to 471 of SEQ ID NO: 26. In the present specification, the antibody is referred to as c2A4.

Examples of the chimeric antibody derived from the rat anti-human GPRC5D antibody 2B1 can include an antibody consisting of a light chain comprising a light chain variable region consisting of amino acid residues 21 to 234 of SEQ ID NO: 30 and a heavy chain comprising a heavy chain variable region consisting of amino acid residues 20 to 472 of SEQ ID NO: 34. One example of such a 2B1-derived chimeric antibody can include an antibody consisting of a light chain consisting of amino acid residues 21 to 234 of SEQ ID NO: 30 and a heavy chain consisting of amino acid residues 20 to 472 of SEQ ID NO: 34. In the present specification, the antibody is referred to as c2B1.

Examples of the chimeric antibody derived from the rat anti-human GPRC5D antibody 7B4 can include an antibody consisting of a light chain comprising a light chain variable region consisting of amino acid residues 21 to 127 of SEQ ID NO: 38 and a heavy chain comprising a heavy chain variable region consisting of amino acid residues 20 to 142 of SEQ ID NO: 42. One example of such a 7B4-derived chimeric antibody can include an antibody consisting of a light chain consisting of amino acid residues 21 to 233 of SEQ ID NO: 38 and a heavy chain consisting of amino acid residues 20 to 472 of SEQ ID NO: 42. In the present specification, the antibody is referred to as c7B4.

Examples of the humanized antibody can include a human-derived antibody containing only complementarity determining regions (CDRs) (Nature (1986) 321, 522-525), a human antibody grafted with the CDR sequences and with some amino acid residues of framework regions by CDR grafting (International Publication No. WO1990/007861)), and an antibody having human antibody amino acid(s) replaced for one or two or more non-human animal antibody-derived amino acid(s) in any of these humanized antibodies.

The humanized antibody derived from each chimeric antibody mentioned above maintains all of 6 CDR sequences derived from the chimeric antibody mentioned above and by extension, the rat antibody, and has ADCC activity. Thus, examples of the antibody that maintains all of 6 CDR sequences shown below include rat antibodies, chimeric antibodies, and humanized antibodies.

The heavy chain variable region of the humanized antibody derived from 2A4 mentioned above maintains heavy chain CDR1 consisting of the amino acid sequence represented by SEQ ID NO: 45 (GYTFTSYY), heavy chain CDR2 consisting of the amino acid sequence represented by SEQ ID NO: 46 (VYPGYGGT), and heavy chain CDR3 consisting of the amino acid sequence represented by SEQ ID NO: 47 (ARRKGIIRGPGYFDY).
The light chain variable region thereof maintains
light chain CDR1 consisting of the amino acid sequence represented by SEQ ID NO: 54 (EGISNS),
light chain CDR2 consisting of the amino acid sequence represented by SEQ ID NO: 55 (GAS), and
light chain CDR3 consisting of the amino acid sequence represented by SEQ ID NO: 56 (QQGYKYPPT).

The heavy chain variable region of the humanized antibody derived from 2B1 mentioned above maintains heavy chain CDR1 consisting of the amino acid sequence represented by SEQ ID NO: 48 (GFSLNTYDMG), heavy chain CDR2 consisting of the amino acid sequence represented by SEQ ID NO: 49 (IWWDDDK), and heavy chain CDR3 consisting of the amino acid sequence represented by SEQ ID NO: 50 (ARIETVRVSRKGFAH).
The light chain variable region thereof maintains
light chain CDR1 consisting of the amino acid sequence represented by SEQ ID NO: 57 (QSVGIN),
light chain CDR2 consisting of the amino acid sequence represented by SEQ ID NO: 58 (GAS), and
light chain CDR3 consisting of the amino acid sequence represented by SEQ ID NO: 59 (LQHGSIPPT).

The heavy chain variable region of the humanized antibody derived from 7B4 mentioned above maintains
heavy chain CDR1 consisting of the amino acid sequence represented by SEQ ID NO: 51 (GYTITSGYD),
heavy chain CDR2 consisting of the amino acid sequence represented by SEQ ID NO: 52 (MSYRGST), and
heavy chain CDR3 consisting of the amino acid sequence represented by SEQ ID NO: 53 (ALTRTYWYNYYYVLDA).
The light chain variable region thereof maintains
light chain CDR1 consisting of the amino acid sequence represented by SEQ ID NO: 60 (QNINKY),
light chain CDR2 consisting of the amino acid sequence represented by SEQ ID NO: 61 (NTN), and
light chain CDR3 consisting of the amino acid sequence represented by SEQ ID NO: 62 (LQRNSWYT).

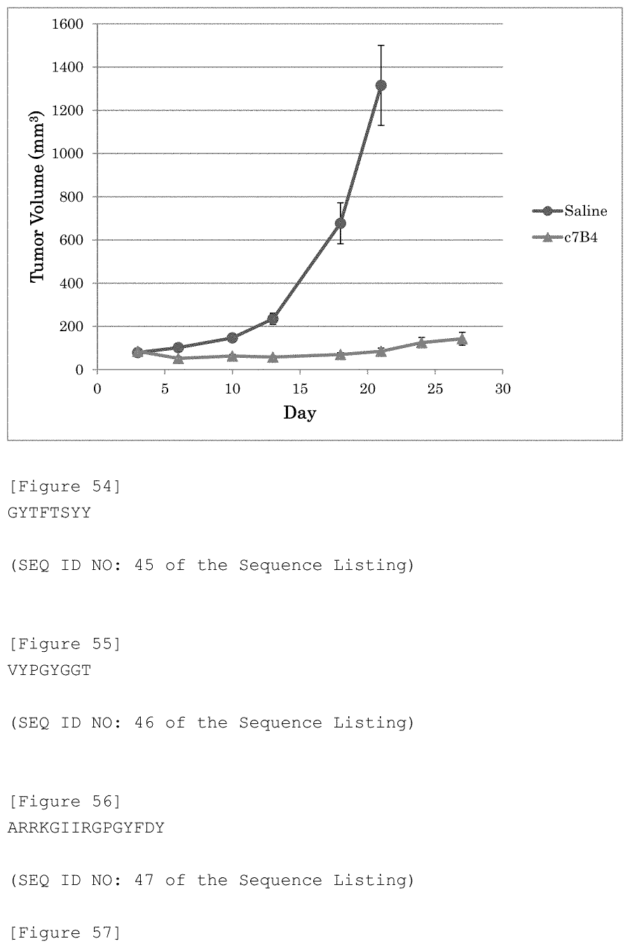

The amino acid sequences of the CDRs mentioned above are also described in FIGS. 54 to 71.

In the present invention, the positions and lengths of CDRs were determined according to the definition of IMGT (Developmental and Comparative Immunology 27 (2003) 55-77).

Preferred examples of the humanized antibody can include an antibody comprising
a light chain variable region comprising any one amino acid sequence represented by
amino acid residues 21 to 127 of the amino acid sequence represented by SEQ ID NO: 64,
amino acid residues 21 to 127 of the amino acid sequence represented by SEQ ID NO: 66,
amino acid residues 21 to 127 of the amino acid sequence represented by SEQ ID NO: 68,
amino acid residues 21 to 127 of the amino acid sequence represented by SEQ ID NO: 70, and
amino acid residues 21 to 127 of the amino acid sequence represented by SEQ ID NO: 72, and
a heavy chain variable region comprising any one amino acid sequence represented by
amino acid residues 21 to 142 of the amino acid sequence represented by SEQ ID NO: 74,
amino acid residues 21 to 142 of the amino acid sequence represented by SEQ ID NO: 76,
amino acid residues 21 to 142 of the amino acid sequence represented by SEQ ID NO: 78, and
amino acid residues 21 to 142 of the amino acid sequence represented by SEQ ID NO: 80.

Alternative preferred examples of the humanized antibody can include an antibody comprising
a light chain variable region comprising an amino acid sequence represented by
amino acid residues 21 to 126 of the amino acid sequence represented by SEQ ID NO: 82, or
amino acid residues 21 to 126 of the amino acid sequence represented by SEQ ID NO: 84, and
a heavy chain variable region comprising any one amino acid sequence represented by
amino acid residues 20 to 142 of the amino acid sequence represented by SEQ ID NO: 86,
amino acid residues 20 to 142 of the amino acid sequence represented by SEQ ID NO: 88,
amino acid residues 20 to 142 of the amino acid sequence represented by SEQ ID NO: 90, and
amino acid residues 20 to 142 of the amino acid sequence represented by SEQ ID NO: 92.

Specific preferred examples of the humanized antibody can include an antibody comprising any one combination of a heavy chain variable region and a light chain variable region of
a heavy chain variable region comprising an amino acid sequence represented by amino acid residues 21 to 142 of the amino acid sequence represented by SEQ ID NO: 74, and a light chain variable region comprising an amino acid sequence represented by amino acid residues 21 to 127 of the amino acid sequence represented by SEQ ID NO: 64,
a heavy chain variable region comprising an amino acid sequence represented by amino acid residues 21 to 142 of the amino acid sequence represented by SEQ ID NO: 74, and a light chain variable region comprising an amino acid sequence represented by amino acid residues 21 to 127 of the amino acid sequence represented by SEQ ID NO: 66,
a heavy chain variable region comprising an amino acid sequence represented by amino acid residues 21 to 142 of the amino acid sequence represented by SEQ ID NO: 76, and a light chain variable region comprising an amino acid sequence represented by amino acid residues 21 to 127 of the amino acid sequence represented by SEQ ID NO: 66,
a heavy chain variable region comprising an amino acid sequence represented by amino acid residues 21 to 142 of the amino acid sequence represented by SEQ ID NO: 76, and a light chain variable region comprising an amino acid sequence represented by amino acid residues 21 to 127 of the amino acid sequence represented by SEQ ID NO: 68,
a heavy chain variable region comprising an amino acid sequence represented by amino acid residues 21 to 142 of the amino acid sequence represented by SEQ ID NO: 76, and a light chain variable region comprising an amino acid sequence represented by amino acid residues 21 to 127 of the amino acid sequence represented by SEQ ID NO: 70,
a heavy chain variable region comprising an amino acid sequence represented by amino acid residues 21 to 142 of the amino acid sequence represented by SEQ ID NO: 76, and a light chain variable region comprising an amino acid sequence represented by amino acid residues 21 to 127 of the amino acid sequence represented by SEQ ID NO: 72,
a heavy chain variable region comprising an amino acid sequence represented by amino acid residues 21 to 142 of the amino acid sequence represented by SEQ ID NO: 78, and a light chain variable region comprising an amino acid sequence represented by amino acid residues 21 to 127 of the amino acid sequence represented by SEQ ID NO: 68,
a heavy chain variable region comprising an amino acid sequence represented by amino acid residues 21 to 142 of the amino acid sequence represented by SEQ ID NO: 78, and a light chain variable region comprising an amino acid sequence represented by amino acid residues 21 to 127 of the amino acid sequence represented by SEQ ID NO: 70,
a heavy chain variable region comprising an amino acid sequence represented by amino acid residues 21 to 142 of the amino acid sequence represented by SEQ ID NO: 78, and a light chain variable region comprising an amino acid sequence represented by amino acid residues 21 to 127 of the amino acid sequence represented by SEQ ID NO: 72,
a heavy chain variable region comprising an amino acid sequence represented by amino acid residues 21 to 142 of the amino acid sequence represented by SEQ ID NO: 80, and a light chain variable region comprising an amino acid sequence represented by amino acid residues 21 to 127 of the amino acid sequence represented by SEQ ID NO: 64,
a heavy chain variable region comprising an amino acid sequence represented by amino acid residues 21 to 142 of the amino acid sequence represented by SEQ ID NO: 80, and a light chain variable region comprising an amino acid sequence represented by amino acid residues 21 to 127 of the amino acid sequence represented by SEQ ID NO: 68,
a heavy chain variable region comprising an amino acid sequence represented by amino acid residues 21 to 142 of the amino acid sequence represented by SEQ ID NO: 80, and a light chain variable region comprising an amino acid sequence represented by amino acid residues 21 to 127 of the amino acid sequence represented by SEQ ID NO: 70, and
a heavy chain variable region comprising an amino acid sequence represented by amino acid residues 21 to 142 of the amino acid sequence represented by SEQ ID NO: 80, and a light chain variable region comprising an amino acid sequence represented by amino acid residues 21 to 127 of the amino acid sequence represented by SEQ ID NO: 72.

Alternative specific preferred examples of the humanized antibody can include an antibody comprising any one combination of a heavy chain variable region and a light chain variable region of
a heavy chain variable region comprising an amino acid sequence represented by amino acid residues 20 to 142 of the amino acid sequence represented by SEQ ID NO: 86, and a light chain variable region comprising an amino acid sequence represented by amino acid residues 21 to 126 of the amino acid sequence represented by SEQ ID NO: 84,
a heavy chain variable region comprising an amino acid sequence represented by amino acid residues 20 to 142 of the amino acid sequence represented by SEQ ID NO: 88, and a light chain variable region comprising an amino acid sequence represented by amino acid residues 21 to 126 of the amino acid sequence represented by SEQ ID NO: 84,
a heavy chain variable region comprising an amino acid sequence represented by amino acid residues 20 to 142 of the amino acid sequence represented by SEQ ID NO: 90, and a light chain variable region comprising an amino acid sequence represented by amino acid residues 21 to 126 of the amino acid sequence represented by SEQ ID NO: 82,
a heavy chain variable region comprising an amino acid sequence represented by amino acid residues 20 to 142 of the amino acid sequence represented by SEQ ID NO: 90, and a light chain variable region comprising an amino acid sequence represented by amino acid residues 21 to 126 of the amino acid sequence represented by SEQ ID NO: 84, and
a heavy chain variable region comprising an amino acid sequence represented by amino acid residues 20 to 142 of the amino acid sequence represented by SEQ ID NO: 92, and a light chain variable region comprising an amino acid sequence represented by amino acid residues 21 to 126 of the amino acid sequence represented by SEQ ID NO: 82.

More preferred examples of the humanized antibody can include an antibody comprising any one combination of a heavy chain and a light chain of
a heavy chain comprising amino acid residues 21 to 472 of the amino acid sequence represented by SEQ ID NO: 74, and a light chain comprising amino acid residues 21 to 234 of the amino acid sequence represented by SEQ ID NO: 64,
a heavy chain comprising amino acid residues 21 to 472 of the amino acid sequence represented by SEQ ID NO: 74, and a light chain comprising amino acid residues 21 to 234 of the amino acid sequence represented by SEQ ID NO: 66,
a heavy chain comprising amino acid residues 21 to 472 of the amino acid sequence represented by SEQ ID NO: 76, and a light chain comprising amino acid residues 21 to 234 of the amino acid sequence represented by SEQ ID NO: 66,
a heavy chain comprising amino acid residues 21 to 472 of the amino acid sequence represented by SEQ ID NO: 76, and a light chain comprising amino acid residues 21 to 234 of the amino acid sequence represented by SEQ ID NO: 68,
a heavy chain comprising amino acid residues 21 to 472 of the amino acid sequence represented by SEQ ID NO: 76, and a light chain comprising amino acid residues 21 to 234 of the amino acid sequence represented by SEQ ID NO: 70,
a heavy chain comprising amino acid residues 21 to 472 of the amino acid sequence represented by SEQ ID NO: 76, and a light chain comprising amino acid residues 21 to 234 of the amino acid sequence represented by SEQ ID NO: 72,
a heavy chain comprising amino acid residues 21 to 472 of the amino acid sequence represented by SEQ ID NO: 78, and a light chain comprising amino acid residues 21 to 234 of the amino acid sequence represented by SEQ ID NO: 68,
a heavy chain comprising amino acid residues 21 to 472 of the amino acid sequence represented by SEQ ID NO: 78, and a light chain comprising amino acid residues 21 to 234 of the amino acid sequence represented by SEQ ID NO: 70,
a heavy chain comprising amino acid residues 21 to 472 of the amino acid sequence represented by SEQ ID NO: 78, and a light chain comprising amino acid residues 21 to 234 of the amino acid sequence represented by SEQ ID NO: 72,
a heavy chain comprising amino acid residues 21 to 472 of the amino acid sequence represented by SEQ ID NO: 80, and a light chain comprising amino acid residues 21 to 234 of the amino acid sequence represented by SEQ ID NO: 64,
a heavy chain comprising amino acid residues 21 to 472 of the amino acid sequence represented by SEQ ID NO: 80, and a light chain comprising amino acid residues 21 to 234 of the amino acid sequence represented by SEQ ID NO: 68,
a heavy chain comprising amino acid residues 21 to 472 of the amino acid sequence represented by SEQ ID NO: 80, and a light chain comprising amino acid residues 21 to 234 of the amino acid sequence represented by SEQ ID NO: 70, and
a heavy chain comprising amino acid residues 21 to 472 of the amino acid sequence represented by SEQ ID NO: 80, and a light chain comprising amino acid residues 21 to 234 of the amino acid sequence represented by SEQ ID NO: 72.

Alternative more preferred examples of the humanized antibody can include an antibody comprising any one combination of a heavy chain and a light chain of
a heavy chain comprising amino acid residues 20 to 472 of the amino acid sequence represented by SEQ ID NO: 86, and a light chain comprising amino acid residues 21 to 233 of the amino acid sequence represented by SEQ ID NO: 84,
a heavy chain comprising amino acid residues 20 to 472 of the amino acid sequence represented by SEQ ID NO: 88, and a light chain comprising amino acid residues 21 to 233 of the amino acid sequence represented by SEQ ID NO: 84,
a heavy chain comprising amino acid residues 20 to 472 of the amino acid sequence represented by SEQ ID NO: 90, and a light chain comprising amino acid residues 21 to 233 of the amino acid sequence represented by SEQ ID NO: 82,
a heavy chain comprising amino acid residues 20 to 472 of the amino acid sequence represented by SEQ ID NO: 90, and a light chain comprising amino acid residues 21 to 233 of the amino acid sequence represented by SEQ ID NO: 84, and
a heavy chain comprising amino acid residues 20 to 472 of the amino acid sequence represented by SEQ ID NO: 92, and a light chain comprising amino acid residues 21 to 233 of the amino acid sequence represented by SEQ ID NO: 82.

The rat antibodies, the chimeric antibodies, and the humanized antibodies mentioned above may each contain Fc.

Also, the rat antibodies, the chimeric antibodies, and the humanized antibodies mentioned above may each contain a human immunoglobulin heavy chain constant region.

Further more preferred examples of the humanized antibody can include an antibody comprising aforementioned heavy chain variable region and light chain variable region, preferably heavy chain variable region and light chain variable region of a humanized antibody derived from 2B1, and
i) a heavy chain constant region comprising an amino acid sequence represented by amino acid residues 147 to 475 of the amino acid sequence represented by SEQ ID NO: 199, and a light chain constant region comprising an amino acid sequence represented by amino acid residues 131 to 237 of the amino acid sequence represented by SEQ ID NO: 203,
ii) a heavy chain constant region comprising an amino acid sequence represented by amino acid residues 147 to 475 of the amino acid sequence represented by SEQ ID NO: 201, and a light chain constant region comprising an amino acid sequence represented by amino acid residues 131 to 237 of the amino acid sequence represented by SEQ ID NO: 205,
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217.

Particularly preferred examples of the humanized antibody can include an antibody comprising a heavy chain variable region comprising an amino acid sequence represented by amino acid residues 20 to 142 of the amino acid sequence represented by SEQ ID NO: and a light chain variable region comprising an amino acid sequence represented by amino acid residues 21 to 127 of the amino acid sequence represented by SEQ ID NO: 72, and comprising
i) a heavy chain constant region comprising an amino acid sequence represented by amino acid residues 147 to 475 of the amino acid sequence represented by SEQ ID NO: 199, and a light chain constant region comprising an amino acid sequence represented by amino acid residues 131 to 237 of the amino acid sequence represented by SEQ ID NO: 203,
ii) a heavy chain constant region comprising an amino acid sequence represented by amino acid residues 147 to 475 of the amino acid sequence represented by SEQ ID NO: 201, and a light chain constant region comprising an amino acid sequence represented by amino acid residues 131 to 237 of the amino acid sequence represented by SEQ ID NO: 205,
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217.

Of these, more preferable antibody can include an antibody comprising a heavy chain variable region comprising an amino acid sequence represented by amino acid residues 20 to 142 of the amino acid sequence represented by SEQ ID NO: 76 and a light chain variable region comprising an amino acid sequence represented by amino acid residues 21 to 127 of the amino acid sequence represented by SEQ ID NO: 72, and comprising
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217.

Specific examples of these can include an antibody comprising

a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217,
or

a heavy chain comprising an amino acid sequence represented by amino acid residues 24 to 475 of the amino acid sequence represented by SEQ ID NO: 237 and a light chain comprising an amino acid sequence represented by amino acid residues 24 to 237 of the amino acid sequence represented by SEQ ID NO: 217.

Alternative Further more preferred examples of the humanized antibody can include an antibody comprising aforementioned heavy chain variable region and light chain variable region,preferably heavy chain variable region and light chain variable region of a humanized antibody derived from 2B1 and natural or mutated Fc.

Particularly preferred examples of these humanized antibody can include an antibody comprising a heavy chain variable region comprising an amino acid sequence represented by amino acid residues 20 to 142 of the amino acid sequence represented by SEQ ID NO: 76 and a light chain variable region comprising an amino acid sequence represented by amino acid residues 21 to 127 of the amino acid sequence represented by SEQ ID NO: 72, and natural or mutated Fc.

Specific examples of these can include an antibody comprising an amino acid sequence represented by amino acid residues 24 to 271 of the amino acid sequence represented by SEQ ID NO: 223, and natural or mutated Fc.

The human antibody is not particularly limited as long as the antibody binds to human GPRC5D. Examples thereof can also include a human antibody that binds to the same site as in the case of the humanized antibody of the present invention. Examples thereof include a human antibody that binds to the same site as in the case of h2B1H2L5.

Examples of the human antibody of the present invention include an antibody comprising a heavy chain variable region and a light chain variable region described in any one of the following ((A) to (D):
(A)
a heavy chain variable region comprising
heavy chain CDR1 consisting of the amino acid sequence represented by SEQ ID NO: 111,
heavy chain CDR2 consisting of the amino acid sequence represented by SEQ ID NO: 112, and
heavy chain CDR3 consisting of the amino acid sequence represented by SEQ ID NO: 113,
and
a light chain variable region comprising
light chain CDR1 consisting of the amino acid sequence represented by SEQ ID NO: 114,
light chain CDR2 consisting of the amino acid sequence represented by SEQ ID NO: 115, and
light chain CDR3 consisting of the amino acid sequence represented by SEQ ID NO: 116,
(B)
a heavy chain variable region comprising
heavy chain CDR1 consisting of the amino acid sequence represented by SEQ ID NO: 117,
heavy chain CDR2 consisting of the amino acid sequence represented by SEQ ID NO: 118, and
heavy chain CDR3 consisting of the amino acid sequence represented by SEQ ID NO: 119,
and
a light chain variable region comprising
light chain CDR1 consisting of the amino acid sequence represented by SEQ ID NO: 120,
light chain CDR2 consisting of the amino acid sequence represented by SEQ ID NO: 121, and
light chain CDR3 consisting of the amino acid sequence represented by SEQ ID NO: 122,
(C)
a heavy chain variable region comprising
heavy chain CDR1 consisting of the amino acid sequence represented by SEQ ID NO: 123,
heavy chain CDR2 consisting of the amino acid sequence represented by SEQ ID NO: 124, and
heavy chain CDR3 consisting of the amino acid sequence represented by SEQ ID NO: 125,
and
a light chain variable region comprising
light chain CDR1 consisting of the amino acid sequence represented by SEQ ID NO: 126,
light chain CDR2 consisting of the amino acid sequence represented by SEQ ID NO: 127, and
light chain CDR3 consisting of the amino acid sequence represented by SEQ ID NO: 128, and
(D)
a heavy chain variable region comprising
heavy chain CDR1 consisting of the amino acid sequence represented by SEQ ID NO: 129,
heavy chain CDR2 consisting of the amino acid sequence represented by SEQ ID NO: 130, and
heavy chain CDR3 consisting of the amino acid sequence represented by SEQ ID NO: 131, and
a light chain variable region comprising
light chain CDR1 consisting of the amino acid sequence represented by SEQ ID NO: 132,
light chain CDR2 consisting of the amino acid sequence represented by SEQ ID NO: 133, and
light chain CDR3 consisting of the amino acid sequence represented by SEQ ID NO: 134.

The amino acid sequences of the CDRs mentioned above are also described in FIGS. 124 to 147.

Preferred examples of the human antibody can include an antibody comprising
a heavy chain variable region comprising any one of the amino acid sequence represented by SEQ ID NO: 97, the amino acid sequence represented by SEQ ID NO: 101,
the amino acid sequence represented by SEQ ID NO: 105, and
the amino acid sequence represented by SEQ ID NO: 109,
and
a light chain variable region comprising any one of the amino acid sequence represented by SEQ ID NO: 99, the amino acid sequence represented by SEQ ID NO: 103,
the amino acid sequence represented by SEQ ID NO: 107, and
the amino acid sequence represented by SEQ ID NO: 135 or an antigen-binding fragment of the antibody.

Specific preferred examples of the human antibody can include an antibody comprising any one combination of a heavy chain variable region and a light chain variable region of
a heavy chain variable region comprising the amino acid sequence represented by SEQ ID NO: 97, and a light chain variable region comprising the amino acid sequence represented by SEQ ID NO: 99,
a heavy chain variable region comprising the amino acid sequence represented by SEQ ID NO: 101, and a light chain variable region comprising the amino acid sequence represented by SEQ ID NO: 103,
a heavy chain variable region comprising the amino acid sequence represented by SEQ ID NO: 105, and a light chain variable region comprising the amino acid sequence represented by SEQ ID NO: 107, and
a heavy chain variable region comprising the amino acid sequence represented by SEQ ID NO: 109, and a light chain variable region comprising the amino acid sequence represented by SEQ ID NO: 135.
Such an antibody includes single chain Fv (also referred to as scFv) (Example 10)-4).

Another example of the human antibody includes an IgG type antibody comprising the aforementioned light and heavy chain variable regions linked to human immunoglobulin light chain constant (CL) and heavy chain constant (CH1 and Fc) regions, respectively. Examples of such an IgG type human antibody include an antibody comprising any one combination of a heavy chain and a light chain of
a heavy chain comprising the amino acid sequence represented by SEQ ID NO: 144, and a light chain comprising the amino acid sequence represented by SEQ ID NO: 145,
a heavy chain comprising the amino acid sequence represented by SEQ ID NO: 146, and a light chain comprising the amino acid sequence represented by SEQ ID NO: 147,
a heavy chain comprising the amino acid sequence represented by SEQ ID NO: 148, and a light chain comprising the amino acid sequence represented by SEQ ID NO: 149, and
a heavy chain comprising the amino acid sequence represented by SEQ ID NO: 150, and a light chain comprising the amino acid sequence represented by SEQ ID NO: 151.

The anti-GPRC5D antibody of the present invention may be an antibody comprised of portions derived from a plurality of different antibodies as long as the antibody binds to human GPRC5D. Examples of such an antibody can include an antibody comprising heavy and/or light chains exchanged among a plurality of different antibodies, an antibody comprising full-length heavy and/or light chains exchanged there among, an antibody comprising variable or constant regions exchanged there among, and an antibody comprising all or some CDRs exchanged there among. The heavy and light chain variable regions of the chimeric antibody may be derived from different anti-GPRC5D antibodies of the present invention. Heavy chain CDR1 to CDR3 and light chain CDR1 to CDR3 in the heavy and light chain variable regions of the humanized antibody may be derived from two or more different anti-GPRC5D antibodies of the present invention. Heavy chain CDR1 to CDR3 and light chain CDR1 to CDR3 in the heavy and light chain variable regions of the human antibody may be a combination of CDRs carried by two or more different anti-GPRC5D antibodies of the present invention.

The anti-GPRC5D antibody of the present invention includes a single chain immunoglobulin comprising full-length heavy and light chain sequences of the antibody linked via an appropriate linker (Lee, H-S, et al., Molecular Immunology (1999) 36, 61-71; and Shirrmann, T. et al., mAbs (2010), 2 (1), 1-4). Such a single chain immunoglobulin can be dimerized to thereby maintain a structure and activities similar to those of the antibody, which is originally a tetramer.

The anti-GPRC5D antibody of the present invention or antigen-binding fragment of the antibody also includes an antibody or an antigen-binding fragment of the antibody that comprises an amino acid sequence encoded by a nucleotide sequence contained in a polynucleotide hybridizing under stringent conditions to a complementary strand of a polynucleotide comprising a nucleotide sequence encoding an amino acid sequence contained in the anti-GPRC5D antibody of the present invention or antigen-binding fragment of the antibody, and binds to human GPRC5D.

The anti-GPRC5D antibody of the present invention or antigen-binding fragment of the antibody may be an antibody or an antigen-binding fragment of the antibody that comprises an amino acid sequence of a heavy chain variable region and/or an amino acid sequence of a light chain variable region 90%, 91%, 92%, 93%, 94%, 95%, 96%, 97%, 98%, or 99% or more identical to amino acid sequence(s) contained in the aforementioned antibody or antigen-binding fragment of the antibody according to any one of (8) to (12) and (18) to (20), and binds to human GPRC5D.

Compared to the positions and lengths of light chain variable regions using a definition of IMGT, the positions and lengths of light chain variable regions using a definition different from IMGT may additionally contain one or two or more amino acid(s), for example, arginine (R) and/or glycine (G), in the carboxyl termini of the amino acid sequences of the light chain variable regions. An antibody or an antigen-binding fragment thereof having such a light chain variable region is also encompassed by the antibody of the present invention or antigen-binding fragment thereof.

The ability of the antibody, etc. of the present invention to bind to GPRC5D, particularly, human and/or cynomolgus monkey GPRC5D, may be optimized by introducing a mutation to the antigen-binding fragment of the anti-GPRC5D antibody of the present invention. Specific examples of the method for introducing a mutation can include random mutagenesis using error-prone PCR, site directed amino acid mutagenesis using an NNK library, site directed mutagenesis using structure information, and combinations thereof.

### 3-2. Antibody Mutant of Anti-GPRC5D Antibody

The antibody mutant of the anti-GPRC5D antibody of the present invention can preferably exhibit, for example, reduced sensitivity to protein degradation or oxidation, a preserved or improved biological activity or function or suppressed reduction or change in biological activity or function, an improved or adjusted ability to bind to the antigen, or physicochemical or functional properties imparted thereto. Proteins are known to vary in their functions or activities due to change in a particular amino acid side chain present on the surface thereof. Examples of such a case include the deamidation of an asparagine side chain and the isomerization of an aspartic acid side chain. An antibody derived from the anti-GPRC5D antibody of the present invention by replacement with another amino acid in order to prevent such change in amino acid side chain is also included in the scope of the antibody mutant of the present invention.

Examples of the antibody mutant of the present invention can include an antibody having an amino acid sequence derived from the amino acid sequence of the original antibody by conservative amino acid substitution. The conservative amino acid substitution is a substitution that occurs in an amino acid group related to amino acid side chains.

Preferred amino acid groups are as follows: an acidic group including aspartic acid and glutamic acid; a basic group including lysine, arginine, and histidine; a nonpolar group including alanine, valine, leucine, isoleucine, proline, phenylalanine, methionine, and tryptophan; and an uncharged polar family including glycine, asparagine, glutamine, cysteine, serine, threonine, and tyrosine. Other preferred amino acid groups are as follows: an aliphatic hydroxy group including serine and threonine; an amide-containing group including asparagine and glutamine; an aliphatic group including alanine, valine, leucine, and isoleucine; and an aromatic group including phenylalanine, tryptophan, and tyrosine. Such amino acid substitution in the antibody mutant is preferably performed without reducing the antigen binding activity of the original antibody.

The anti-GPRC5D antibody of the present invention, the antigen-binding fragment thereof, the variant thereof, or the molecule of the present invention also encompasses: an antibody mutant that has an amino acid sequence derived from the amino acid sequence of the antibody of the present invention such as 2A4, 2B1, or 7B4 by conservative amino acid substitution and/or any other mutation, and binds to human GPRC5D, an antigen-binding fragment thereof, a molecule comprising the antibody mutant or antigen-binding fragment, etc.; a mouse antibody, a rat antibody, a chimeric antibody, a humanized antibody, or a human antibody that comprises a CDR having an amino acid sequence in which conservative amino acid substitution and/or any other mutation occurs in the amino acid sequence of any of CDRH1 to CDRH3 and CDRL1 to CDRL3 derived from the antibody of the present invention such as 2A4, 2B1, or 7B4, and binds to human GPRC5D, an antigen-binding fragment thereof, a molecule comprising the antibody or antigen-binding fragment, etc.; an antibody mutant that has an amino acid sequence derived from the amino acid sequence of the antibody of the present invention such as C2037, C3048, C3015, or C3022 by conservative amino acid substitution, and binds to human GPRC5D, an antigen-binding fragment thereof, a molecule comprising the antibody mutant or antigen-binding fragment, etc.; and chimeric antibody or a human antibody that comprises a CDR having an amino acid sequence in which a conservative amino acid mutation occurs in the amino acid sequence of any of CDRH1 to CDRH3 and CDRL1 to CDRL3 derived from the antibody of the present invention such as C2037, C3048, C3015, or C3022, and binds to human GPRC5D, an antigen-binding fragment thereof, a molecule comprising the antibody or antigen-binding fragment, etc.

### 3-3. Antigen-Binding Fragment of Anti-GPRC5D Antibody

According to one aspect, the present invention provides an antigen-binding fragment of the anti-GPRC5D antibody of the present invention. The antigen-binding fragment of the anti-GPRC5D antibody of the present invention encompasses antigen-binding fragments of chimeric antibodies, humanized antibodies, or human antibodies. The antigen-binding fragment of the antibody means a fragment that maintains at least antigen binding activity among the functions of the antibody, or a modified form thereof. Examples of such functions of the antibody can generally include antigen binding activity, antigen activity-regulating activity (e.g., agonistic activity), activity of internalizing an antigen into cells, and activity of inhibiting or promoting the interaction between an antigen and its interacting substance.

The antigen-binding fragment of the antibody is not particularly limited as long as the fragment of the antibody maintains at least antigen binding activity among the activities of the antibody. Examples of such an antigen-binding fragment of the antibody include, but are not limited to, Fab, Fab′, F(ab′)₂, single chain Fab (scFab) comprising the carboxy terminus of a Fab light chain and the amino terminus of a Fab heavy chain linked via an appropriate linker, Fv, single chain Fv (scFv) comprising heavy and light chain Fvs linked via an appropriate linker, and single domain antibody (sdAb; also called nanobody) having a single heavy chain variable region and lacking a light chain sequence (Muyldemans S. et al., Protein Eng., (1994), 7 (9), 1129-35; and Hamers-Casterman C. et al., Nature (1993), 363 (6428), 446-448). The antigen-binding fragment of the antibody of the present invention is also meant to include a molecule comprising the antigen-binding fragment of the antibody of the present invention as well as other portions, such as scFab and scFv retaining a linker portion.

### 3-4 Modified Form of Anti-GPRC5D Antibody or Antigen-Binding Fragment Thereof and Conjugates

The present invention provides a modified form of the antibody or antigen-binding fragment thereof. The modified form of the antibody of the present invention or antigen-binding fragment thereof means an antibody of the present invention or an antigen-binding fragment thereof provided with chemical or biological modification. The chemically modified form includes, for example, a form having an amino acid skeleton conjugated with a chemical moiety, and a form having a chemically modified N-linked or O-linked carbohydrate chain. The biologically modified form includes, for example, a form that has undergone post-translational modification (e.g., N-linked or O-linked glycosylation, processing of an amino-terminal or carboxyl-terminal region, deamidation, isomerization of aspartic acid, or oxidation of methionine), and a form containing a methionine residue added to the amino terminus by expression using prokaryotic host cells. Such a modified form is also meant to include a form labeled to permit detection or isolation of the antibody or the antigen of the present invention, for example, an enzyme-labeled form, a fluorescently labeled form, or an affinity-labeled form. Such a modified form of the antibody of the present invention or antigen-binding fragment thereof is useful for improvement of the stability or blood retention of the original antibody of the present invention or the original antigen-binding fragment thereof, reduction in antigenicity, detection or isolation of the antibody or the antigen, etc.

Examples of the chemical moiety contained in the chemically modified form can include water-soluble polymers such as polyethylene glycol(PEG), ethylene glycol/propylene glycol copolymers, carboxymethylcellulose, dextran, and polyvinyl alcohol.

Examples of the biologically modified form can include a form modified by enzymatic treatment, cell treatment, or the like, a form fused with another peptide, such as a tag, added by gene recombination, and a form prepared from host cells expressing an endogenous or exogenous sugar chain-modifying enzyme.

Such a modification may be made at an arbitrary position or a desired position in the antibody or antigen-binding fragment thereof. Alternatively, the same or two or more different modifications may be made at one or two or more positions therein.

The deletion in these heavy chain sequences or the modification in the heavy or light chain sequences, however, rarely influences the ability of the antibody to bind to the antigen and its effector functions (complement activation, antibody dependent cytotoxic effects, etc.).

Thus, the present invention also encompasses an antibody that has received the deletion or the modification. Examples thereof can include a deletion variant derived from a heavy chain by the deletion of 1 or 2 amino acid(s) at its carboxyl terminus (Journal of Chromatography A, 705: 129-134 (1995)), an amidated form of the deletion variant having a heavy chain that lacks two amino acid residues (glycine and lysine) at the carboxy terminus and instead has an amidated proline residue at the carboxy terminus (Analytical Biochemistry, 360: 75-83 (2007)), and a modified antibody having a pyroglutamylated amino-terminal glutamine or glutamic acid residue in its heavy or light chain (International Publication No. WO2013/147153) (these are collectively referred to as a “deletion variant”). However, the deletion variant at the carboxyl terminus of the antibody heavy or light chain according to the present invention is not limited to the types described above as long as the deletion variant maintains the ability to bind to the antigen and the effector functions. When the antibody according to the present invention comprises two or more chains (e.g., heavy chains), the two or more chains (e.g., heavy chains) may be heavy chains of any one type selected from the group consisting of the full-length heavy chain and the deletion variants described above, or may be a combination of heavy chains of any two types selected therefrom. The quantitative ratio of each deletion variant or the ratio of the number of molecules thereof may be influenced by the type of cultured mammalian cells producing the antibody according to the present invention, and culture conditions. Examples of such a case can include the deletion of one carboxyl-terminal amino acid residue each in both of the two heavy chains as main components of the antibody according to the present invention.

The antibody of the present invention or antigen-binding fragment thereof (e.g., contained in the molecule, the multispecific molecule, the bispecific molecule, etc. of the present invention), even if 1 to several amino acid(s) derived from, for example, an expression vector and/or a signal sequence, is added to the amino terminus and/or the carboxyl terminus (and a portion or the whole thereof is modified as described above), is included in the scope of the modified form of the antibody of the present invention or the modified form of the antigen-binding fragment thereof as long as the desired antigen binding activity is maintained. A molecule comprising such a modified form of the antibody or antigen-binding fragment is also included in the scope of the molecule of the present invention.

In the present invention, the scope of the “antibody or antigen-binding fragment thereof” is meant to also include the “deletion variant” and the “modified form” of the “antibody or antigen-binding fragment thereof”, and mixtures therewith. The “antibody or antigen-binding fragment thereof” contained in the molecule, the multispecific molecule, the bispecific molecule, etc. of the present invention is meant to also include the “deletion variant” and the modified form” of the “antibody or antigen-binding fragment thereof”, and mixtures therewith.

The present invention also encompasses conjugates formed by the aforementioned antibodies linked with other molecules via linkers (immunoconjugates). Examples of such an antibody-drug complex in which the antibody is conjugated with a radioactive material or a compound (drug) having a pharmacological action can include ADC (antibody-drug conjugate) ([Methods Mol Biol. (2013) 1045: 1-27; Nature Biotechnology (2005) 23, p.1137-1146)).

In addition, the present invention also encompasses conjugates comprising these antibodies connected to other functional polypeptides. Examples of such an antibody-peptide complex can include a complex of the antibody and an albumin-binding polypeptide (Protein Eng Des Sel. (2012) (2): 81-8).

These modified forms of the antibody, antibodies that have undergone the sugar chain modification thus regulated, and conjugates are encompassed by the antibody of the present invention. Antigen-binding fragments of these modified forms of the antibody, antibodies that have undergone the sugar chain modification thus regulated, and conjugates are encompassed by the antigen-binding fragment of the antibody of the present invention.

s3-5. Antibody Binding to Same Site

An antibody that binds to a site on human GPRC5D bound by the antibody provided by the present invention or antigen-binding fragment of the antibody is also included in the antibody of the present invention or antigen-binding fragment of the antibody. The antibody that binds to the same site on the human GPRC5D antigen as in the case of a certain antibody means another antibody that binds to the same site on the antigen molecule as that recognized by the antibody. If a second antibody binds to a partial peptide or a partial three-dimensional structure on an antigen molecule bound by a first antibody, the first and second antibodies are determined as binding to the same site.

Alternatively, the first and second antibodies are determined as binding to the same site by confirming that the second antibody competes with the first antibody for binding to the antigen, i.e., the second antibody interferes with the binding of the first antibody to the antigen, even if the peptide sequence or three-dimensional structure of the specific binding site is not determined.

When the first and second antibodies bind to the same site, the second antibody has an exceedingly high probability of having the same activity as the first antibody.

The antibody binding site can be determined by a method well known by those skilled in the art, such as immunoassay. For example, a series of peptides are prepared by appropriately cleaving the amino acid sequence of the antigen from its carboxyl terminus or amino terminus, and the reactivity of the antibody thereto is studied to roughly determine a recognition site. Then, shorter peptides are synthesized, and the reactivity of the antibody to these peptides can be studied to thereby determine the binding site. The antigen fragment peptides can be prepared using a technique such as gene recombination or peptide synthesis.

### 3-6. Polynucleotide, Vector, and Cell of the Present Invention

The present invention also provides a polynucleotide comprising a nucleotide sequence (e.g., SEQ ID NO: 63, 65, 67, 69, 71, 73, 75, 77, 79, 81, 83, 85, 87, 89, or 91) encoding the amino acid sequence of the anti-GPRC5D antibody of the present invention or antigen-binding fragment thereof, a vector comprising the polynucleotide, a cell comprising the polynucleotide or the vector, and a cell producing the anti-GPRC5D antibody of the present invention or antigen-binding fragment thereof, etc. Such a polynucleotide, vector (a circular form such as a plasmid, and a noncircular form including vectors integrated in chromosomes), and cell are useful in the production of the anti-GPRC5D antibody or antigen-binding fragment thereof mentioned later.

The polynucleotide of the present invention may contain an arbitrary nucleotide sequence, in addition to the nucleotide sequence encoding the amino acid sequence of the anti-GPRC5D antibody or antigen-binding fragment thereof. For example, in addition to a polynucleotide comprising the nucleotide sequence (SEQ ID NO: 63, 65, 67, 69, 71, 73, 75, 77, 79, 81, 83, 85, 87, 89, 91, etc. of the Sequence Listing) encoding the amino acid sequence of the anti-GPRC5D antibody of the present invention or antigen-binding fragment thereof, a nucleotide sequence encoding the amino acid sequence of an activity signal transduction molecule peptide and/or a nucleotide sequence encoding the amino acid sequence of a co-stimulatory molecule are also part of the aspect of the polynucleotide of the present invention. The present invention also provides an artificial immunocyte having binding activity against tumor cells through a chimeric antigen receptor (CAR) expressed by the transfection of an immunocyte (e.g., a T cell, a NK cell, or a monocyte) with such a polynucleotide (hereinafter, also referred to as the artificial immunocyte of the present invention).

CAR is a chimeric protein having scFv comprising tandemly bound light and heavy chains derived from a monoclonal antibody that recognizes a surface antigen on tumor cells, and an activity signal transduction molecule (e.g., CD3ζ for T cell receptors or a receptor FcRγ for immunoglobulin molecules) at its amino terminus and carboxy terminus, respectively, between which an extracellular hinge domain, a transmembrane domain, a co-stimulatory molecule activating immunocytes, and the like are connected. In the cell of the present invention, the monoclonal antibody that recognizes a surface antigen on tumor cells is the anti-GPRC5D antibody of the present invention. The immunocyte of the present invention expressing CAR recognizes a GPRC5D protein on the surface of tumor via the anti-GPRC5D antibody of the present invention in the form of scFv while the activation of the immunocyte itself is induced by the intracellular activity signal transduction molecule to attack tumor cells.

In the case of using a T cell as the immunocyte to be transfected with the polynucleotide of the present invention, the chimeric antigen receptor (CAR) expressed on cell surface by the transfection of the T cell with this polynucleotide can cause a GPRC5D-expressing cell and the T cell to come close to each other to induce T cell activation-mediated cytotoxicity to the GPRC5D-expressing cell, i.e., cytotoxicity by the redirection of the T cell. The present invention also provides such a T cell expressing CAR (hereinafter, also referred to as the T cell of the present invention).

In other words, the T cell of the present invention can be redirected to tumor cells expressing GPRC5D to induce cytotoxicity to the tumor cells.

The activity signal transduction molecule is transfected into the immunocyte in order to transduce signals from an immunocyte receptor into the cell. In the case of using, for example, a T cell as the immunocyte, examples of the activity signal transduction molecule include CD3ζ, DAP12, and FcRγ.

The co-stimulatory molecule is transfected into the immunocyte in order to more strongly activate the immunocyte. In the case of using, for example, a T cell as the immunocyte, examples of the co-stimulatory molecule include CD2, CD27, CD28, CD49d, CD134, CD152, CD154, ICOS, 4-1BB, and RANKL.

### 4. Molecule Having Antigen Binding Activity

The molecule of the present invention comprises the anti-GPRC5D antibody of the present invention or antigen-binding fragment thereof.

Further, the molecule of the present invention can comprise, for example, a signal sequence, a tag for purification, etc., amino-terminal Gly, a drug linker portion of ADC, an albumin-binding polypeptide, a polymer such as PEG, an antibody other than the anti-GPRC5D antibody, an antigen-binding fragment thereof, and a protein having antigen binding activity without having an immunoglobulin skeleton, which will be described later. Examples of the antibody other than the anti-GPRC5D antibody include an anti-CD3 antibody. The molecule of the present invention includes a multispecific molecule described later.

### 4-1. Multispecific Molecule

The multispecific molecule of the present invention is a molecule having two or more antigen-binding sites. Specifically, the multispecific molecule of the present invention is a molecule capable of binding to two or more epitopes different from each other on one molecule, or epitopes different from each other on two or more molecules, and comprises a plurality of antigen-binding fragments different from each other. Examples of such a multispecific molecule include, but are not limited to, IgG-type multispecific molecules and multispecific molecules having two or more types of variable regions, for example, antibody fragments such as tandem scFv, single chain diabodies, diabodies, and triabodies, and antibody fragments linked by a covalent bond or a noncovalent bond. The multispecific molecule may contain Fc.

The multispecific molecule of the present invention comprises the anti-GPRC5D antibody of the present invention or antigen-binding fragment of the antibody. The multispecific molecule of the present invention comprises the anti-GPRC5D antibody of the present invention or antigen-binding fragment thereof and 1 or 2 or more antibody(ies) that is different from the anti-GPRC5D antibody and binds to an epitope absent in GPRC5D and present in another antigen, or antigen-binding fragment(s) of the antibody(ies).

Examples of the antigen-binding fragment of the anti-GPRC5D antibody can include Fab, F(ab)′, Fv, scFv, and sdAb.

The multispecific molecule of the present invention specifically binds to GPRC5D or may further bind to a target such as an Fc receptor on effector cells.

Examples of the antibody different from the anti-GPRC5D antibody that can be contained in the multispecific molecule of the present invention include an anti-CD3 antibody.

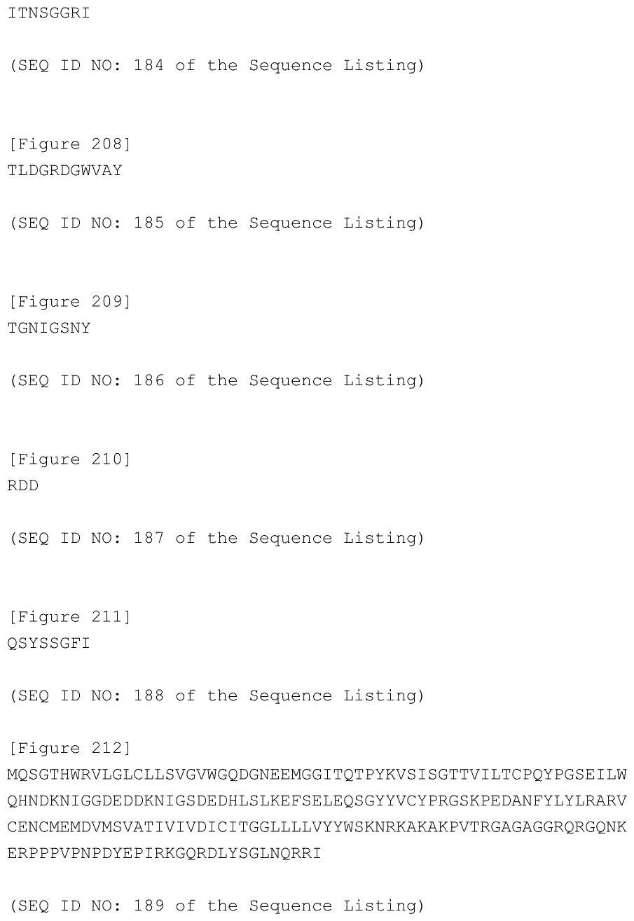

As a preferred example, the anti-CD3 antibody or antigen-binding fragment thereof that can be contained in the multispecific molecule of the present invention retains a heavy chain CDR1 comprising the amino acid sequence represented by SEQ ID NO: 183 (FIG. 206) (GVTFNYYG),
heavy chain CDR2 comprising the amino acid sequence represented by SEQ ID NO: 238 (FIG. 276) (ITXₐₐXₐₐGGRI) (wherein each of the first Xₐₐ and the second Xₐₐ is an arbitrary natural amino acid residue; hereinafter, the first Xₐₐ and the second Xₐₐ in the heavy chain CDR2 are also referred to as X₁ and X₂, respectively), and
CDRH3 comprising the amino acid sequence represented by SEQ ID NO: 185 (FIG. 208) (TLDGRDGWVAY).

The light chain variable region contained in a preferred humanized anti-CD3 antibody of the present invention or antigen-binding fragment of the antibody retains
light chain CDR1 comprising the amino acid sequence represented by SEQ ID NO: 186 (FIG. 209) (TGNIGSNY), light chain CDR2 comprising the amino acid sequence represented by SEQ ID NO: 239 (FIG. 277) (RXₐₐD) (wherein Xₐₐ is an arbitrary natural amino acid residue; hereinafter, Xₐₐ in the light chain CDR2 is also referred to as X₃), and
light chain CDR3 comprising the amino acid sequence represented by SEQ ID NO: 188 (FIG. 211) (QSYSSGFI).

In the aforementioned heavy chain CDR2 (ITX₁X₂GGRI), preferably, X₁ is selected from the group consisting of (A, E, G, H, I, L, T, V, R, and S), and X₂ is S; or X₁ is N, and X₂ is selected from the group consisting of (E, R, F, Y, L, V, I, K, and T).

In the aforementioned light chain CDR2 (RX₃D), preferably, X₃ is selected from the group consisting of (Q, A, G, S, N, and D).

In the aforementioned heavy chain CDR2 (ITX₁X₂GGRI), more preferably, X₁ is selected from the group consisting of (R and S), and X₂ is S.

In the aforementioned light chain CDR2 (RX₃D), more preferably X₃ is selected from the group consisting of (Q, A, G, S, N, and D).

Preferable examples of humanized anti-CD3 antibody or antigen-binding fragment of the antibody which can be comprised in a molecule of the present invention comprise a heavy chain variable region comprising amino acid residues shown in SEQ ID NO: 240 (FIG. 278) and a light chain variable region comprising amino acid residues shown in SEQ ID NO: 241 (FIG. 279), SEQ ID NO: 242 (FIG. 280), or SEQ ID NO: 243 (FIG. 281), wherein X₁ in amino acid residues shown in SEQ ID NO: 240 is selected from the group consisting of (A, E, G, H, I, L, T, V, R, and S), and X₂ is S; or X₁ is N, and X₂ is selected from the group consisting of (E, R, F, Y, L, V, I, K, and T).

More preferable examples of humanized anti-CD3 antibody or antigen-binding fragment of the antibody which can be comprised in a molecule of the present invention comprise a heavy chain variable region comprising amino acid residues shown in SEQ ID NO: 240 (FIG. 278) and a light chain variable region comprising amino acid residues shown in SEQ ID NO: 241 (FIG. 279), SEQ ID NO: 242 (FIG. 280), or SEQ ID NO: 243 (FIG. 281), wherein X₁ in amino acid residues shown in SEQ ID NO: 240 is selected from the group consisting of (R, S), and X₂ is S; and X₃ is selected from the group consisting of is N, and X₂ is selected from the group consisting of (Q, A, G, S, N, and D).

Specific examples of the preferred humanized anti-CD3 antibody of the present invention or antigen-binding fragment of the antibody include an antibody or an antigen-binding fragment of the antibody that comprises
a heavy chain variable region comprising
the amino acid sequence of heavy chain CDR1 represented by SEQ ID NO: 183 (GVTFNYYG),
the amino acid sequence of heavy chain CDR2 represented by SEQ ID NO: 184 (ITNSGGRI), and
the amino acid sequence of heavy chain CDR3 represented by SEQ ID NO: 185 (TLDGRDGWVAY), and
a light chain variable region comprising
the amino acid sequence of light chain CDR1 represented by SEQ ID NO: 186 (TGNIGSNY),
the amino acid sequence of light chain CDR2 represented by SEQ ID NO: 187 (RDD), and
the amino acid sequence of light chain CDR3 represented by SEQ ID NO: 188 (QSYSSGFI),
and binds to human CD3 and cynomolgus monkey CD3. The positions and lengths of these CDRs of the anti-CD3 antibody were determined according to the definition of IMGT.

The anti-CD3 antibody or antigen-binding fragment thereof contained in the multispecific molecule of the present invention having the CDRs mentioned above, and their variable regions (hereinafter, also referred to as the anti-CD3 antibody, etc. of the present invention) bind to an Ig-like domain present in the extracellular region of the ε chain of the human CD3 complex. Furthermore, these also bind to an Ig-like domain present in the extracellular region of the ε chain of the cynomolgus monkey CD3 complex.

Epitopes present in the extracellular region of the ε chain of the human CD3 complex bound by the anti-CD3 antibody, etc. contained in the multispecific molecule of the present invention contain the following amino acids:
Ser55, Glu56, Leu58, Trp59, Asn65, Ile66, Ser77, Asp78, Arg101, Gly102, Ser103, Lys104, and Pro105.

Preferably, the anti-CD3 antibody, etc. contained in the multispecific molecule of the present invention can maintain binding to human CD3 by binding to an epitope region containing at least 7 amino acids selected from these 13 amino acids.

When an antibody is adjacent to these amino acids at a distance within 4 angstroms, such an antibody can be confirmed to have the same epitope specificity as that of the anti-CD3 antibody, etc. contained in the multispecific molecule of the present invention. On the other hand, among these epitope amino acids, Arg101, Gly102, Ser103, Lys104, and Pro105 are also epitope residues that interact with an anti-CD3 antibody OKT3 or UCHT1 known in the art (Lars Kjer-Nielsen et al., PNAS (2004); and Kelly L Arnett et al., PNAS (2004)). However, OKT3 or UCHT1 binds to human CD3, but does not bind to cynomolgus monkey CD3.

Such an antibody that binds to human CD3 and cynomolgus monkey CD3 or antigen-binding fragment of the antibody contained in the multispecific molecule of the present invention can be subjected to various tests on efficacy or safety using primates, particularly, cynomolgus monkeys, useful for the nonclinical development (preclinical development) of pharmaceutical products, and is thus preferred. Also, the antibody, etc. binding to human CD3 and cynomolgus monkey CD3 has cytotoxic activity and is useful, either alone or as the molecule of the present invention, in the treatment or prevention of diseases such as cancers in cynomolgus monkeys. The pharmaceutical composition will be described later.

The anti-CD3 antibody may be a nonhuman animal antibody, a chimeric antibody, a humanized antibody, or a human antibody. The anti-CD3 antibody is preferably a humanized antibody or a human antibody.

Examples of the antigen-binding fragment of the anti-CD3 antibody include Fab, F(ab)′, Fv, scFv, and sdAb.

Examples of the anti-CD3 antibody or antigen-binding fragment of the antibody comprising the CDRs described above include a humanized antibody or an antigen-binding fragment of the antibody comprising a heavy chain variable region comprising the amino acid sequence represented by SEQ ID NO: 240(FIG. 278), and a light chain variable region comprising an amino acid sequence represented by any one of SEQ ID NOs: 241(FIG. 279), 242(FIG. 280), and 243(FIG. 281). Specific examples thereof include a humanized antibody or an antigen-binding fragment of the antibody comprising a heavy chain variable region comprising the amino acid sequence represented by SEQ ID NO: 155, and a light chain variable region comprising an amino acid sequence represented by any one of SEQ ID NOs: 156, 158, and 160.

Specific examples of the anti-CD3 antibody or antigen-binding fragment of the antibody include an antibody or an antigen-binding fragment of the antibody comprising the amino acid sequence represented by SEQ ID NO: 180, 181, or 182. More specific examples thereof include an antibody or an antigen-binding fragment of the antibody comprising amino acid residues 2 to 243 of SEQ ID NO: 180,
an antibody or an antigen-binding fragment of the antibody comprising amino acid residues 2 to 243 of SEQ ID NO: 181, and
an antibody or an antigen-binding fragment of the antibody comprising amino acid residues 2 to 241 of SEQ ID NO: 182.

The anti-CD3 antibody mentioned above may be a humanized antibody or a human antibody comprising a human immunoglobulin constant region or Fc. Fc can be mutated Fc.

Preferable examples of multispecific molecule of the present invention, comprising anti-GPRC5D antibody or an antigen-binding fragment of the antibody and anti-CD3 antibody or an antigen-binding fragment of the antibody can comprise anti-GPRC5D antibody or an antigen-binding fragment of the antibody derived from aforementioned 2B1 and comprise an antibody that binds to human CD3 and cynomolgus monkey CD3 or antigen-binding fragment of the antibody comprising
a heavy chain variable region comprising an amino acid sequence represented by amino acid residues 25 to 142 of the amino acid sequence represented by SEQ ID NO: 207 and a light chain variable region comprising an amino acid sequence represented by amino acid residues 24 to 132 of the amino acid sequence represented by SEQ ID NO: 209,
a heavy chain variable region comprising an amino acid sequence represented by amino acid residues 25 to 142 of the amino acid sequence represented by SEQ ID NO: 211 and a light chain variable region comprising an amino acid sequence represented by amino acid residues 24 to 130 of the amino acid sequence represented by SEQ ID NO: 213,
a heavy chain variable region comprising an amino acid sequence represented by amino acid residues 2 to 119 of the amino acid sequence represented by SEQ ID NO: 244 and a light chain variable region comprising an amino acid sequence represented by amino acid residues 135 to 243 of the amino acid sequence represented by SEQ ID NO: 244,
a heavy chain variable region comprising an amino acid sequence represented by amino acid residues 2 to 119 of the amino acid sequence represented by SEQ ID NO: 245 and a light chain variable region comprising an amino acid sequence represented by amino acid residues 135 to 241 of the amino acid sequence represented by SEQ ID NO: 245,
a heavy chain variable region comprising an amino acid sequence represented by amino acid residues 2 to 119 of the amino acid sequence represented by SEQ ID NO: 246 and a light chain variable region comprising an amino acid sequence represented by amino acid residues 135 to 243 of the amino acid sequence represented by SEQ ID NO: 246,
a heavy chain variable region comprising an amino acid sequence represented by amino acid residues 2 to 119 of the amino acid sequence represented by SEQ ID NO: 247 and a light chain variable region comprising an amino acid sequence represented by amino acid residues 135 to 243 of the amino acid sequence represented by SEQ ID NO: 247,
a heavy chain variable region comprising an amino acid sequence represented by amino acid residues 2 to 119 of the amino acid sequence represented by SEQ ID NO: 248 and a light chain variable region comprising an amino acid sequence represented by amino acid residues 135 to 243 of the amino acid sequence represented by SEQ ID NO: 248,
a heavy chain variable region comprising an amino acid sequence represented by amino acid residues 2 to 119 of the amino acid sequence represented by SEQ ID NO: 249 and a light chain variable region comprising an amino acid sequence represented by amino acid residues 135 to 243 of the amino acid sequence represented by SEQ ID NO: 249,
a heavy chain variable region comprising an amino acid sequence represented by amino acid residues 2 to 119 of the amino acid sequence represented by SEQ ID NO: 250 and a light chain variable region comprising an amino acid sequence represented by amino acid residues 135 to 243 of the amino acid sequence represented by SEQ ID NO: 250,
a heavy chain variable region comprising an amino acid sequence represented by amino acid residues 2 to 119 of the amino acid sequence represented by SEQ ID NO: 251 and a light chain variable region comprising an amino acid sequence represented by amino acid residues 135 to 243 of the amino acid sequence represented by SEQ ID NO: 251,
a heavy chain variable region comprising an amino acid sequence represented by amino acid residues 2 to 119 of the amino acid sequence represented by SEQ ID NO: 252 and a light chain variable region comprising an amino acid sequence represented by amino acid residues 135 to 243 of the amino acid sequence represented by SEQ ID NO: 252,
a heavy chain variable region comprising an amino acid sequence represented by amino acid residues 2 to 119 of the amino acid sequence represented by SEQ ID NO: 253 and a light chain variable region comprising an amino acid sequence represented by amino acid residues 135 to 243 of the amino acid sequence represented by SEQ ID NO: 253,
a heavy chain variable region comprising an amino acid sequence represented by amino acid residues 2 to 119 of the amino acid sequence represented by SEQ ID NO: 254 and a light chain variable region comprising an amino acid sequence represented by amino acid residues 135 to 243 of the amino acid sequence represented by SEQ ID NO: 254, or
a heavy chain variable region comprising an amino acid sequence represented by amino acid residues 2 to 119 of the amino acid sequence represented by SEQ ID NO: 255 and a light chain variable region comprising an amino acid sequence represented by amino acid residues 135 to 243 of the amino acid sequence represented by SEQ ID NO: 255.

Preferable molecule of these wherein the antibody that binds to human GPRC5D or an antigen-binding fragment of the antibody comprises a heavy chain variable region comprising an amino acid sequence represented by amino acid residues 20 to 142 of the amino acid sequence represented by SEQ ID NO: and a light chain variable region comprising an amino acid sequence represented by amino acid residues 21 to 127 of the amino acid sequence represented by SEQ ID NO: 72, and comprises
i) a heavy chain constant region comprising an amino acid sequence represented by amino acid residues 147 to 475 of the amino acid sequence represented by SEQ ID NO: 199, and a light chain constant region comprising an amino acid sequence represented by amino acid residues 131 to 237 of the amino acid sequence represented by SEQ ID NO: 203,
ii) a heavy chain constant region comprising an amino acid sequence represented by amino acid residues 147 to 475 of the amino acid sequence represented by SEQ ID NO: 201, and a light chain constant region comprising an amino acid sequence represented by amino acid residues 131 to 237 of the amino acid sequence represented by SEQ ID NO: 205,
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217,
and
the antibody that binds to human CD3 and cynomolgus monkey CD3 or antigen-binding fragment of the antibody comprises mutated Fc.

More preferable molecule of these wherein the antibody that binds to human GPRC5D or an antigen-binding fragment of the antibody comprises a heavy chain variable region comprising an amino acid sequence represented by amino acid residues 20 to 142 of the amino acid sequence represented by SEQ ID NO: and a light chain variable region comprising an amino acid sequence represented by amino acid residues 21 to 127 of the amino acid sequence represented by SEQ ID NO: 72, and comprises
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217,
and
the antibody that binds to human CD3 and cynomolgus monkey CD3 or antigen-binding fragment of the antibody comprises mutated Fc.

Specific preferable examples of multispecific molecule of the present invention can comprises

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 219 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 221 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 225 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 227 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 229 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 231 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 233 and mutated Fc; or

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 235 and mutated Fc.

Specific examples of more preferable examples of multispecific molecule of the present invention can comprise

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 225 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 227 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 229 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 231 and mutated Fc;

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and the antibody that binds to human CD3 and cynomolgus
monkey CD3 or antigen-binding fragment of the antibody comprising an amino acid sequence represented by amino acid residues 24 to 266 of the amino acid sequence represented by SEQ ID NO: 233 and mutated Fc; or

the antibody that binds to human GPRC5D or an antigen-binding fragment of the antibody comprising a heavy chain comprising an amino acid sequence represented by amino acid residues 24 to 475 of the amino acid sequence represented by SEQ ID NO: 215 and a light chain comprising an amino acid sequence represented by amino acid residues 24 to 237 of the amino acid sequence represented by SEQ ID NO: 217, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 235 and mutated Fc.

Alternative example of molecule wherein the antibody that binds to human GPRC5D or an antigen-binding fragment of the antibody comprises a heavy chain variable region comprising an amino acid sequence represented by amino acid residues 20 to 142 of the amino acid sequence represented by SEQ ID NO: 76 and a light chain variable region comprising an amino acid sequence represented by amino acid residues 21 to 127 of the amino acid sequence represented by SEQ ID NO: 72, and comprises
i) a heavy chain constant region comprising an amino acid sequence represented by amino acid residues 147 to 475 of the amino acid sequence represented by SEQ ID NO: 199, and a light chain constant region comprising an amino acid sequence represented by amino acid residues 131 to 237 of the amino acid sequence represented by SEQ ID NO: 203,
ii) a heavy chain constant region comprising an amino acid sequence represented by amino acid residues 147 to 475 of the amino acid sequence represented by SEQ ID NO: 201, and a light chain constant region comprising an amino acid sequence represented by amino acid residues 131 to 237 of the amino acid sequence represented by SEQ ID NO: 205,
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217,
and
the antibody that binds to human CD3 and cynomolgus monkey CD3 or antigen-binding fragment of the antibody comprises
v) a heavy chain constant region comprising an amino acid sequence represented by amino acid residues 147 to 471 of the amino acid sequence represented by SEQ ID NO: 207, and a light chain constant region comprising an amino acid sequence represented by amino acid residues 133 to 238 of the amino acid sequence represented by SEQ ID NO: 209, or
vi) a heavy chain constant region comprising an amino acid sequence represented by amino acid residues 143 to 471 of the amino acid sequence represented by SEQ ID NO: 211, and a light chain constant region comprising an amino acid sequence represented by amino acid residues 131 to 236 of the amino acid sequence represented by SEQ ID NO: 213.

More preferable example of the molecule can comprise
iii) a heavy chain constant region comprising an amino acid sequence represented by amino acid residues 147 to 475 of the amino acid sequence represented by SEQ ID NO: 215, and a light chain constant region comprising an amino acid sequence represented by amino acid residues 131 to 237 of the amino acid sequence represented by SEQ ID NO: 217,
or
iv) a heavy chain constant region comprising an amino acid sequence represented by amino acid residues 147 to 475 of the amino acid sequence represented by SEQ ID NO: 237, and a light chain constant region comprising an amino acid sequence represented by amino acid residues 131 to 237 of the amino acid sequence represented by SEQ ID NO: 217,
and
the antibody that binds to human CD3 and cynomolgus monkey CD3 or antigen-binding fragment of the antibody comprises
v) a heavy chain constant region comprising an amino acid sequence represented by amino acid residues 147 to 471 of the amino acid sequence represented by SEQ ID NO: 207, and a light chain constant region comprising an amino acid sequence represented by amino acid residues 133 to 238 of the amino acid sequence represented by SEQ ID NO: 209, or
vi) a heavy chain constant region comprising an amino acid sequence represented by amino acid residues 143 to 471 of the amino acid sequence represented by SEQ ID NO: 211, and a light chain constant region comprising an amino acid sequence represented by amino acid residues 131 to 236 of the amino acid sequence represented by SEQ ID NO: 213.

Specific example of the molecule can comprise the antibody that binds to human GPRC5D or an antigen-binding fragment of the antibody comprising a heavy chain comprising an amino acid sequence represented by amino acid residues 24 to 475 of the amino acid sequence represented by SEQ ID NO: 119 and a light chain comprising an amino acid sequence represented by amino acid residues 24 to 237 of the amino acid sequence represented by SEQ ID NO: 203, and
the antibody that binds to human CD3 and cynomolgus monkey CD3 or antigen-binding fragment of the antibody comprising a heavy chain comprising an amino acid sequence represented by amino acid residues 25 to 471 of the amino acid sequence represented by SEQ ID NO: 207 and a light chain comprising an amino acid sequence represented by amino acid residues 24 to 238 of the amino acid sequence represented by SEQ ID NO: 209
or
the antibody that binds to human GPRC5D or an antigen-binding fragment of the antibody comprising a heavy chain comprising an amino acid sequence represented by amino acid residues 24 to 475 of the amino acid sequence represented by SEQ ID NO: 201 and a light chain comprising an amino acid sequence represented by amino acid residues 24 to 237 of the amino acid sequence represented by SEQ ID NO: 205, and
the antibody that binds to human CD3 and cynomolgus monkey CD3 or antigen-binding fragment of the antibody comprising a heavy chain comprising an amino acid sequence represented by amino acid residues 25 to 471 of the amino acid sequence represented by SEQ ID NO: 211 and a light chain comprising an amino acid sequence represented by amino acid residues 24 to 236 of the amino acid sequence represented by SEQ ID NO: 213.

Alternative molecule can comprise the antibody that binds to human GPRC5D or an antigen-binding fragment of the antibody comprises a heavy chain variable region comprising an amino acid sequence represented by amino acid residues 20 to 142 of the amino acid sequence represented by SEQ ID NO: 76 and a light chain variable region comprising an amino acid sequence represented by amino acid residues 21 to 127 of the amino acid sequence represented by SEQ ID NO: 72, and mutated Fc, and
the antibody that binds to human CD3 and cynomolgus monkey CD3 or antigen-binding fragment of the antibody comprises mutated Fc.

Specific example of these can comprise

the antibody that binds to human GPRC5D or an antigen-binding fragment of the antibody comprising amino acid sequence represented by amino acid residues 24 to 271 of the amino acid sequence represented by SEQ ID NO: 223 and mutated Fc, and
the antibody that binds to human CD3 and cynomolgus monkey CD3 or antigen-binding fragment of the antibody comprising an amino acid sequence represented by amino acid residues 24 to 266 of the amino acid sequence represented by SEQ ID NO: 219 and mutated Fc
or
the antibody that binds to human GPRC5D or an antigen-binding fragment of the antibody comprising amino acid sequence represented by amino acid residues 24 to 271 of the amino acid sequence represented by SEQ ID NO: 223 and mutated Fc, and
the antibody that binds to human CD3 and cynomolgus monkey CD3 or antigen-binding fragment of the antibody comprising an amino acid sequence represented by amino acid residues 24 to 264 of the amino acid sequence represented by SEQ ID NO: 221 and mutated Fc.

The molecule of the present invention also encompasses a molecule comprising a moiety of an anti-CD3 antibody or an antigen-binding fragment thereof that comprises an amino acid sequence encoded by a nucleotide sequence contained in a polynucleotide hybridizing under stringent conditions to a complementary strand of a polynucleotide comprising a nucleotide sequence encoding an amino acid sequence contained in the anti-CD3 antibody mentioned above or antigen-binding fragment of the antibody, and binds to human CD3 and cynomolgus monkey CD3, and a moiety of an anti-GPRC5D antibody or an antigen-binding fragment thereof that binds to human GPRC5D and preferably further binds to cynomolgus monkey GPRC5D.

The molecule of the present invention also encompasses a molecule comprising a moiety of an anti-CD3 antibody or an antigen-binding fragment thereof that comprises an amino acid sequence of a heavy chain variable region and/or an amino acid sequence of a light chain variable region 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more identical to the amino acid sequence of the heavy chain variable region and/or the amino acid sequence of the light chain variable region contained in the anti-CD3 antibody mentioned above or antigen-binding fragment of the antibody, and binds to human CD3 and cynomolgus monkey CD3, and a moiety of an anti-GPRC5D antibody or an antigen-binding fragment thereof that binds to human GPRC5D and preferably further binds to cynomolgus monkey GPRC5D.

The molecule of the present invention also encompasses a molecule that comprises an anti-CD3 antibody or an antigen-binding fragment of the antibody having an amino acid sequence derived from an amino acid sequence contained in the anti-CD3 antibody or antigen-binding fragment of the antibody by the substitution, deletion, or modification of 1 to several amino acid(s), and binds to human CD3 and cynomolgus monkey CD3 and to human GPRC5D and preferably further binds to cynomolgus monkey GPRC5D. Examples of such an anti-CD3 antibody or antigen-binding fragment of the antibody can include a deletion variant derived from a heavy chain by the deletion of 1 or 2 amino acid(s) at its carboxyl terminus (Journal of Chromatography A, 705: 129-134 (1995)), an amidated form of the deletion variant having a heavy chain that lacks two amino acid residues (glycine and lysine) at the carboxy terminus and instead has an amidated proline residue at the carboxy terminus (Analytical Biochemistry, 360: 75-83 (2007)), and a modified antibody having a pyroglutamylated amino-terminal glutamine or glutamic acid residue in its heavy or light chain (International Publication No. WO2013/147153) (these are collectively referred to as a “deletion variant”). However, the deletion variant at the carboxyl terminus of the heavy or light chain of the anti-CD3 antibody or antigen-binding fragment of the antibody contained in the molecule of the present invention is not limited to the types described above as long as the deletion variant maintains the ability to bind to the antigen and the effector functions. When the antibody contained in the molecule of the present invention comprises two or more chains (e.g., heavy chains, the two or more chains (e.g., heavy chains) may be heavy chains of any one type selected from the group consisting of the full-length heavy chain and the deletion variants described above, or may be a combination of heavy chains of any two or more types selected therefrom. The quantitative ratio of each deletion variant or the ratio of the number of molecules thereof may be influenced by the type of cultured mammalian cells producing the molecule of the present invention, and culture conditions.

Examples of such a case can include the deletion of one carboxyl-terminal amino acid residue each in both of the two heavy chains as main components of the molecule of the present invention.

### 4-2. Bispecific Molecule of the Present Invention

Preferred examples of the multispecific molecule of the present invention can include bispecific molecules. The term “bispecific” means capability of binding to two epitopes different from each other on one molecule, or epitopes different from each other on two or more molecules. An antibody or an antigen-binding fragment having such bispecificity is encompassed by the present invention.

The bispecific molecule of the present invention binds to GPRC5D and binds to an epitope that is absent in GPRC5D and present in another antigen. More specifically, such a bispecific molecule (i) binds to an epitope on GPRC5D (epitope 1) and (ii) binds to an epitope different from the epitope 1 on GPRC5D (epitope 2), or binds to an epitope on an antigen other than GPRC5D (epitope 3).

For example, in a tandem scFv-type bispecific molecule typified by BiTE, an antigen-binding site in the heavy chain variable region of a first antibody and an antigen-binding site in the light chain variable region of the first antibody are linked either via a linker or directly without a linker to form a first polypeptide. Also, an antigen-binding site in the heavy chain variable region of a second antibody and an antigen-binding site in the light chain variable region of the second antibody are linked either via a linker or directly without a linker to form a second polypeptide. The first polypeptide and the second polypeptide are linked either via a linker or directly without a linker. Alternatively, the first polypeptide and the second polypeptide may be linked via an additional molecule.

In a diabody-type bispecific molecule, an antigen-binding site in the heavy chain variable region of a first antibody and an antigen-binding site in the light chain variable region of a second antibody are linked either via a linker or directly without a linker. Also, an antigen-binding site in the light chain variable region of the first antibody and an antigen-binding site in the heavy chain variable region of the second antibody are linked either via a linker or directly without a linker. Also, a bispecific molecule may be prepared by the further dimerization of diabody-type bispecific molecules. In addition, the diabody-type bispecific molecule may be linked to one single chain or both chains of Fc via a linker (diabody-Fc-type bispecific molecule).

In a dual scFv-type bispecific molecule, two scFvs binding to different epitopes are linked to two chains of dimeric Fc, respectively, either via linkers or directly without linkers. Alternatively, two types of scFvs binding to different epitopes are linked to CH and CL, respectively, via linkers and further linked to two chains of dimeric Fc, respectively, via linkers. Dual scFv type bispecific molecule is described as Dual type bispecific molecule or Dual type.

In an IgG-type bispecific molecule, two Fabs binding to different epitopes are linked to two chains of dimeric Fc, respectively, either via linkers or directly without linkers. IgG-type bispecific molecule is described as Full-size Antibody(FSA) type bispecific molecule or FSA type.

Alternatively, the bispecific molecule of the present invention may be a bispecific molecule in which Fab of a first antibody and scFv of a second antibody are linked to two chains of dimeric Fc, respectively, either via linkers or directly without linkers. Such byspecific antibody is described as Hybrid type bispecific antibody of Hybrid type.

The scFv and the Fab contained in the bispecific molecule of the present invention are preferably scFv and Fab of a humanized antibody or a human antibody, and the Fc is preferably Fc of a human antibody.

The linker also includes a single chain polypeptide or a single chain oligopeptide, or synthetic products such as PEG, nucleotides, sugar chains, and compounds. In addition, any linker known in the art may be used without particular limitations as long as the linker links two polypeptides.

The length of the linker is 5 to 30 amino acids for, for example, a peptide linker. When the bispecific molecule contains a plurality of linkers, all the peptide linkers used may have the same length or the peptide linkers used may have different lengths.

Examples of the peptide linker include a (Gly⋅Gly⋅Gly⋅Gly⋅Ser) repeat. 1 to several amino acid residues other than Gly and Ser may be added thereto.

Examples of the antibody that binds to an epitope on an antigen other than GPRC5D (epitope 3) or antigen-binding fragment thereof contained in the bispecific molecule of the present invention can include the aforementioned anti-CD3 antibody or antigen-binding fragment thereof.

Examples of the bispecific molecule of the present invention can include a molecule in which the anti-CD3 antibody or antigen-binding fragment of the antibody is bound with the anti-GPRC5D antibody of the present invention or antigen-binding fragment of the antibody via a linker or without a linker. Preferred examples of such a molecule can include a molecule in which the anti-CD3 antibody or antigen-binding fragment of the antibody and the anti-GPRC5D antibody of the present invention or antigen-binding fragment of the antibody, each of which is scFv, are bound with each other via a linker or without a linker.

Preferred specific examples of such a molecule can include a molecule that has an amino acid sequence represented by any one of SEQ ID NOs: 171 to 179, and binds to human CD3 and cynomolgus monkey CD3 and to human GPRC5D and preferably further binds to cynomolgus monkey GPRC5D.

### 5. Production of Antibody and Molecule

### 5-1. Method Using Hybridoma

The anti-GPRC5D antibody of the present invention can be obtained by use of a routine method which involves immunizing an animal with GPRC5D or an arbitrary polypeptide selected from the amino acid sequence of GPRC5D and collecting and purifying the antibody produced in vivo. According to a method known in the art (e.g., Kohler and Milstein, Nature (1975) 256, p. 495-497; and Kennet, R. ed., Monoclonal Antibodies, p. 365-367, Plenum Press, N.Y. (1980)), antibody-producing cells that produce antibodies against GPRC5D are fused with myeloma cells to thereby establish hybridomas, from which monoclonal antibodies can also be obtained. Specific examples of such a method are described in, for example, International Publication No. WO09/48072 (published on Apr. 16, 2009).

The organism species of the antigen GPRC5D is not limited to a human, and the animal can also be immunized with GPRC5D derived from a nonhuman animal such as a mouse or a rat. In this case, the obtained antibody that binds to nonhuman GPRC5D can be tested for its cross-reactivity with human GPRC5D to thereby select an antibody applicable to human diseases.

The anti-CD3 antibody that can be contained in the molecule of the present invention can also be obtained by use of a routine method which involves immunizing an animal with CD3 or an arbitrary polypeptide selected from the amino acid sequence of CD3 and collecting and purifying the antibody produced in vivo. According to a method known in the art, antibody-producing cells that produce antibodies against CD3 are fused with myeloma cells to thereby establish hybridomas, from which monoclonal antibodies can also be obtained.

The organism species of the antigen CD3 is not limited to a human, and the animal can also be immunized with CD3 derived from a nonhuman animal such as a mouse or a rat. The obtained antibody that binds to nonhuman CD3 can be tested for its cross-reactivity with human CD3 to thereby select an antibody applicable to human diseases.

### 5-2. Cell Immunization Method

Cells expressing the native antigen, cells expressing the recombinant antigen or its fragment, or the like, can be used as immunogens to thereby prepare an antibody by the hybridoma method described above.

Examples of the cells expressing the native GPRC5D can include human plasma cells, human multiple myeloma patient-derived primary cultured cells, and human multiple myeloma patient-derived cultured cell lines. Examples of the cells expressing the native GD3 can include human thymus cells and T lymphocytes.

Such cells are used in an amount of 1×10⁵ to 1×10⁹ cells, preferably 1×10⁶ to 1×10⁸ cells, more preferably 0.5 to 2×10⁷ cells, even more preferably 1×10⁷ cells, per immunization shot. The number of cells used for immunization can be changed according to the expression level of antigen. The immunogens are generally administered intraperitoneally and may be administered through an intradermal route or the like.

### 5-3. DNA Immunization Method

The anti-GPRC5D antibody of the present invention and the anti-CD3 antibody that can be contained in the molecule of the present invention (hereinafter, these antibodies are also collectively referred to as the antibody of the present invention) can also be obtained by use of a DNA immunization method. This method involves transfecting an animal (e.g., mouse or rat) individual with an antigen expression plasmid and expressing the antigen in the individual to thereby induce immunity against the antigen. Examples of the transfection approach include a method of directly injecting the plasmid to the muscle, a method of injecting a transfection reagent such as a liposome or polyethylenimine to the vein, an approach using a viral vector, an approach of injecting gold particles attached with the plasmid using a gene gun, and a hydrodynamic method of rapidly injecting a plasmid solution in a large amount to the vein.

Actual examples of the rat anti-human GPRC5D antibody thus established can include 2A4, 2B1, and 7B4. The amino acid sequence of the heavy chain variable region of 2A4 is shown in SEQ ID NO: 5 of the Sequence Listing. The amino acid sequence of the light chain variable region of 2A4 is shown in SEQ ID NO: 12 of the Sequence Listing. The amino acid sequence of the heavy chain variable region of 2B1 is shown in SEQ ID NO: 7 of the Sequence Listing. The amino acid sequence of the light chain variable region of 2B1 is shown in SEQ ID NO: 14 of the Sequence Listing. The amino acid sequence of the heavy chain variable region of 7B4 is shown in SEQ ID NO: 9 of the Sequence Listing. The amino acid sequence of the light chain variable region of 7B4 is shown in SEQ ID NO: 16 of the Sequence Listing.

### 5-4. Designing Humanized Antibody

Examples of the humanized antibody can include, but are not limited to, a human-derived antibody having CDRs replaced only with the CDRs of a non-human animal antibody (see Nature (1986), 321, p. 522-525), a human antibody grafted with the CDR sequences and with some amino acid residues of framework regions by CDR grafting (see WO90/07861 and U.S. Pat. No. 6,972,323), and an antibody having human antibody amino acid(s) replaced for one or two or more non-human animal antibody-derived amino acid(s) in any of these humanized antibodies.

### 5-5. Designing Human Antibody

The human antibody means an antibody consisting of the amino acid sequence of a human-derived antibody. The human antibody can be obtained by a method using human antibody-producing mice carrying human genomic DNA fragments comprising human antibody heavy and light chain genes (see e.g., Tomizuka, K. et al., Nature Genetics (1997) 16, 133-143; Kuroiwa, Y. et.al., Nuc. Acids Res. (1998) 26, 3447-3448; Yoshida, H. et. al., Animal Cell Technology: Basic and Applied Aspects vol. 10, 69-73 (Kitagawa, Y., Matuda, T. and Iijima, S. eds.), Kluwer Academic Publishers, 1999; Tomizuka, K. et.al., Proc. Natl. Acad. Sci. USA (2000) 97, 722-727).

Specifically, such human antibody-producing animals can be prepared by disrupting the endogenous immunoglobulin heavy and light chain gene loci of non-human mammals and instead introducing thereto human immunoglobulin heavy and light chain gene loci via yeast artificial chromosome (YAC) vectors or the like. Alternatively, eukaryotic cells may be transformed with cDNAs encoding the heavy and light chains, respectively, of such a human antibody, preferably with vectors comprising the cDNAs, by a gene recombination technique. The transformed cells producing a recombinant human monoclonal antibody can be cultured. This antibody can be obtained from the culture supernatant.

In this context, for example, eukaryotic cells, preferably mammalian cells such as HEK293F cells or CHO cells, can be used as the hosts.

Also, a method for obtaining a phage display-derived human antibody selected from a human antibody library is also known. For example, a phage display method can be used, which involves allowing the variable regions of a human antibody to be expressed as scFv on phage surface and selecting a phage binding to the antigen. The phage selected on the basis of its ability to bind to the antigen can be subjected to gene analysis to thereby determine DNA sequences encoding the variable regions of the human antibody that binds to the antigen. If the DNA sequence of scFv binding to the antigen is determined, an expression vector having this sequence can be prepared and introduced to appropriate hosts to allow them to express the human antibody (WO92/01047, WO92/20791, WO93/06213, WO93/11236, WO93/19172, WO95/01438, WO95/15388, Annu. Rev. Immunol (1994) 12, 433-455).

The method for constructing the human antibody phage library is well known. Genes of human antibody variable regions are amplified using cDNAs collected from human blood, spleen, or lymph node as templates and primers with reference to, for example, J Biol Chem, 274 (26), 18218-30, (1999) or Methods Mol Biol, 178, 59-71, (2002). The amplified variable region genes can be used to prepare scFv with reference to J Immunol Methods, 201 (1), 35-55 (1997).

### 5-6. Production of Antigen-Binding Fragment of Antibody

The antigen-binding fragment of the antibody can be produced by modifying the antibody by a genetic engineering method, followed by expression in appropriate cultured cells.

The method for preparing, for example, scFv, as the antigen-binding fragment of the antibody is well known in the art (see e.g., U.S. Pat. Nos. 4,946,778, 5,260,203, 5,091,513, and 5,455,030). In this scFv, a heavy chain variable region and a light chain variable region are linked via a linker that prevents them from forming a conjugate, preferably a polypeptide linker (Huston, J. S. et al., PNAS (1988), 85, 5879-5883). The heavy chain variable region and the light chain variable region in scFv may be derived from the same antibody or may be derived from different antibodies.

For example, an arbitrary single chain peptide consisting of 5 to 30 residues is used as the polypeptide linker that links these variable regions.

In order to obtain scFv-encoding DNA, of the sequences of DNA encoding the heavy chain or heavy chain variable region of the antibody and DNA encoding the light chain or light chain variable region thereof, each DNA portion encoding the whole or desired amino acid sequence is used as a template and amplified by PCR using a primer pair flanking both ends of the template. Subsequently, DNA encoding the polypeptide linker moiety is further amplified in combination with a primer pair flanking both ends of the DNA so that the obtained fragment can be linked at its ends to the heavy and light chain DNAs, respectively. Alternatively, the DNA encoding the whole scFv region may be obtained by net synthesis.

The scFv-encoding DNA can be used to thereby prepare, according to a routine method, an expression vector containing the DNA and host cells transformed with the expression vector. In addition, the host cells can be cultured, and the scFv can be recovered from the cultures according to a routine method.

Also in order to obtain any other antigen-binding fragment of the antibody, a gene encoding an antigen-binding fragment is obtained according to the method described above and introduced into cells. The antigen-binding fragment of interest can be recovered from cultures of the cells.

The antibody of the present invention may be multimerized to thereby enhance its affinity for the antigen. In this case, antibodies of the same type may be multimerized, or a plurality of antibodies recognizing a plurality of epitopes, respectively, of the same antigen may be multimerized. Examples of methods for multimerizing these antibodies can include the binding of two scFvs to an IgG CH3 domain, the binding thereof to streptavidin, and the introduction of a helix-turn-helix motif.

### 5-7. Gene Recombination

In order to prepare the antibody of the present invention, a polynucleotide (heavy chain nucleotide) comprising a nucleotide sequence encoding the amino acid sequence of its heavy chain and a polynucleotide (light chain nucleotide) comprising a nucleotide sequence encoding the amino acid sequence of its light chain, or a vector having an insert of the heavy chain nucleotide and a vector having an insert of the light chain nucleotide are introduced into host cells, and then the cells are cultured, and the antibody can be recovered from the cultures. The heavy chain nucleotide and the light chain nucleotide may be inserted in one vector.

Prokaryotic or eukaryotic cells can be used as the host cells. In the case of using host eukaryotic cells, animal cells, plant cells, or eukaryotic microbes can be used.

Examples of the animal cells can include mammal-derived cells, i.e., human embryonic kidney cells HEK293F cells (Subedi G P et al., J Vis Exp. (2015) 106), monkey kidney-derived COS cells (Gluzman, Y. Cell (1981), 23, 175-182, ATCC CRL-1650), mouse fibroblast NIH3T3 (ATCC No. CRL-1658), Chinese hamster ovary cells (CHO cells, ATCC CCL-61), dihydrofolate reductase-deficient lines thereof (CHOdhfr−; Urlaub, G. and Chasin, L. A. PNAS (1980), 77, 4126-4220), cells derived from birds such as chickens, and cells derived from insects.

Also, cells modified to enhance the biological activities of antibodies by the modification of sugar chain structures can be used as the hosts. For example, CHO cells modified such that the proportion of sugar chains with fucose unbound with N-acetylglucosamine at their reducing ends is 20% or more among complex-type N-glycoside-linked sugar chains binding to the Fc region of the antibody, may be used to prepare an antibody having enhanced ADCC activity or CDC activity (International Publication No. WO02/31140).

Examples of the eukaryotic microbes can include yeasts. Examples of the prokaryotic cells can include E. coli and Bacillus subtilis.

A signal peptide for the secretion of the antibody of the present invention (monoclonal antibody derived from each animal, rat antibody, mouse antibody, chimeric antibody, humanized antibody, human antibody, etc.) is not limited to the secretory signal of an antibody of the same species, the same type, and the same subtype as the antibody of the present invention or to the secretory signal of the antibody of the present invention itself. Any secretory signal of an antibody of different type or subtype therefrom or any secretory signal of a protein derived from a different eukaryotic species therefrom or a prokaryotic species can be selected and used. The signal peptide is usually not contained in the nucleotide sequences and amino acid sequences of most of mature light chains or mature heavy chains. A secreted antibody, etc. containing the signal peptide is also encompassed by the antibody, etc. of the present invention or the molecule of the present invention.

The obtained antibody, antigen-binding fragment of the antibody, and molecule can be purified until homogeneous so as not to contain other proteins. Usual protein separation and purification methods can be used for the separation and purification of the antibody, antigen-binding fragment of the antibody, and molecule.

The antibody can be separated and purified by appropriately selected or combined approach(es), for example, chromatography columns, filters, ultrafiltration, salting out, dialysis, preparative polyacrylamide gel electrophoresis, and/or isoelectric focusing, though the separation and purification method is not limited thereto.

The separation and purification method can be preferably performed, for example, by preparing an expression vector using a DNA sequence encoding a His tag or a FLAG tag added to the carboxyl terminus of an antibody variable region, transforming cells with this vector, then culturing the cells to express the antibody and antigen-binding fragment of the antibody, and extracting the culture supernatant after the completion of the culture, followed by purification using metal (e.g., Ni or Co) affinity chromatography, anti-FLAG tag antibody columns, gel filtration, ion-exchange chromatography, or the like.

The expressed antibody and antigen-binding fragment of the antibody containing an amino acid sequence of a tag such as a His tag or a FLAG tag is also encompassed by the antibody of the present invention or antigen-binding fragment of the antibody or the molecule of the present invention.

### 5-8. Production of Polyclonal Antibody

The antibody of the present invention may be a polyclonal antibody. The polyclonal antibody can be recovered from cultures of mixed-cultured different antibody-producing cells (WO2004/061104). Alternatively, separately prepared antibodies may be mixed. Antiserum, which is one aspect of the polyclonal antibody, can be prepared by immunizing animals with a desired antigen and recovering serum from the animals according to a standard method.

5-9. Production of Artificial Immunocyte Provided with Binding Specificity for Tumor Cell

An artificial immunocyte provided with binding specificity for tumor cells can be produced by imparting antigen specificity to an immunocyte by transfection with, for example, the gene of the anti-GPRC5D antibody of the present invention. Examples of the immunocyte include T cells, NK cells, and monocytes.

The case of using a T cell as the immunocyte will be described as an example. The T cell can be induced by culturing mononuclear cells recovered from human peripheral blood by a method such as a specific gravity centrifugal method, in a medium in the presence of an anti-CD3 antibody, IL-2, IL-12, or further, an anti-IL-4 antibody or IFN-γ.

Next, this T cell is transfected with a chimeric antigen receptor (CAR) gene comprising the gene of the anti-GPRC5D antibody of the present invention as a component. The CAR gene is typically constituted by a gene of an antibody that recognizes a surface antigen on tumor cells (in the present invention, the anti-GPRC5D antibody) and a gene encoding a co-stimulatory molecule necessary for T cell activation (e.g., co-stimulators such as T cell receptor ζ chain and CD28). The CAR gene comprising the gene of the anti-GPRC5D antibody can be transfected to the T cell using various viral vectors. Examples of such a vector include lentivirus vectors, retrovirus vectors, adenovirus vectors, adeno-associated virus vectors, Sendai virus vectors, and liposomes. A recombinant virus can be prepared from the viral vector having an insert of the gene of the anti-GPRC5D antibody and transfected to the antigen-nonspecifically activated T cell mentioned above. The T cell thus transfected with the gene can be cultured to obtain a T cell provided with specificity for tumor cells.

Whether the T cell of the present invention capable of inducing cytotoxicity by redirection, obtained by the method of the present invention has the antigen specificity thus imparted thereto can be confirmed by coculturing the T cell with inactivated cells obtained by the mitomycin C treatment of antigen-positive tumor cells known to express GPRC5D, and then measuring the amount of IFN-γ or IL-2 in the culture supernatant.

### 5-10. Production of Multispecific Molecule and Bispecific Molecule

Examples of the method for preparing the multispecific molecule and the bispecific molecule of the present invention include a method which involves introducing expression plasmids to host cells, followed by transient expression, a method which involves introducing plasmids to host cells and then selecting a stably expressing cell line by drug selection, followed by constitutive expression, and a method which involves preparing respective antibodies or antigen-binding fragments by any of the methods described above, and then chemically linking these antibodies or fragments using a synthetic peptide linker.

As for the single chain antibody (scFv), examples of the preparation method include a method which involves linking two scFvs via a peptide linker (tandem scFv), a method which involves interchanging the respective domains of two antibodies differing in specificity, and forming a dimer by a noncovalent bond (diabody), a method which involves interchanging the respective domains of two antibodies differing in specificity, and forming a single chain (single chain diabody), and a method which involves preparing single chain diabodies and then forming a dimer by a noncovalent bond (TandAb, U.S. Pat. No. 7,129,330).

The present invention also provides a gene encoding the antibody of the present invention or antigen-binding fragment of the antibody, or a modified form of the antigen, etc., a recombinant vector having an insert of the gene, a cell transfected with the gene or vector, and even a cell producing the antibody of the present invention.

### 6. Pharmaceutical Composition

The present invention provides the anti-GPRC5D antibody or antigen-binding fragment thereof, the polynucleotide, vector, cell, and artificial immunocyte of the present invention, and/or a pharmaceutical composition comprising the molecule comprising at least one of them, as an active ingredient (hereinafter, also referred to as the pharmaceutical composition of the present invention).

The pharmaceutical composition of the present invention is useful in the treatment or prevention of various diseases related to abnormal or increased GPRC5D signals due to overexpression of GPRC5D or its ligand or GPRC5D mutations or gene amplification (hereinafter, these diseases are referred to as “GPRC5D-related diseases”), particularly, various cancers.

Examples of causes of the initiation or exacerbation of such cancers to be treated or prevented can include high expression of GPRC5D, single nucleotide polymorphism (SNP) in an intron of the GPRC5D gene, missense mutations that constitutively activate GPRC5D, and amplification or overexpression of the GPRC5D gene.

Also, the molecule of the present invention or the pharmaceutical composition of the present invention can include cytotoxicity to cells expressing GPRC5D by the redirection of immunocytes such as T cells to the cells. Therefore, the present invention provides a method for inducing cytotoxicity to cells expressing GPRC5D by the redirection of immunocytes such as T cells to the cells, comprising the step of administering the molecule of the present invention or the pharmaceutical composition of the present invention.

Examples of the cancer types to be treated or prevented with the pharmaceutical composition of the present invention can include cancers expressing the GPRC5D protein, for example, breast cancer, endometrial cancer, ovary cancer, lung cancer (e.g., non-small cell lung cancer), stomach cancer, prostate cancer, kidney cancer, liver cancer, pancreatic cancer, colorectal cancer, esophageal cancer, urinary bladder cancer, uterine cervix cancer, blood cancer, lymphoma, and malignant melanoma. Preferred examples thereof can include multiple myeloma expressing the GPRC5D protein.

The treatment or prevention of a GPRC5D-related disease includes, but is not limited to, the prevention of the onset of the disease in an individual expressing the GPRC5D protein, the suppression or inhibition of exacerbation or progression thereof, the alleviation of one or two or more symptoms exhibited by an individual affected with the disease, the suppression or remission of exacerbation or progression thereof, the treatment or prevention of a secondary disease in an individual affected with the disease, etc.

The pharmaceutical composition of the present invention can comprise a therapeutically or prophylactically effective amount of the anti-GPRC5D antibody or antigen-binding fragment thereof, and/or a molecule comprising at least one of them, as an active ingredient and further contain a pharmaceutically acceptable diluent, vehicle, solubilizer, emulsifier, preservative, and/or additive.

The term “therapeutically or prophylactically effective amount” means an amount that exerts therapeutic or prophylactic effects on a particular disease by means of a particular dosage form and administration route, and is used interchangeably with a “pharmacologically effective amount”.

The pharmaceutical composition of the present invention may comprise materials for changing, maintaining, or retaining pH, osmotic pressure, viscosity, transparency, color, tonicity, sterility, or the stability, solubility, sustained release, absorbability, permeability, dosage form, strength, properties, shape, etc., of the composition or the antibody comprised therein (hereinafter, referred to as “pharmaceutical materials”). The pharmaceutical materials are not particularly limited as long as the materials are pharmacologically acceptable. For example, no or low toxicity is a property preferably possessed by these pharmaceutical materials.

Examples of the pharmaceutical materials can include, but are not limited to, the following: amino acids such as glycine, alanine, glutamine, asparagine, histidine, arginine, and lysine; antimicrobial agents; antioxidants such as ascorbic acid, sodium sulfate, and sodium bisulfite; buffers such as phosphate, citrate, or borate buffers, sodium bicarbonate, and Tris-HCl solutions; fillers such as mannitol and glycine; chelating agents such as ethylenediaminetetraacetic acid (EDTA); complexing agents such as caffeine, polyvinylpyrrolidine, β-cyclodextrin, and hydroxypropyl-β-cyclodextrin; bulking agents such as glucose, mannose, and dextrin; other hydrocarbons such as monosaccharides, disaccharides, glucose, mannose, and dextrin; coloring agents; corrigents; diluents; emulsifiers; hydrophilic polymers such as polyvinylpyrrolidine; low-molecular-weight polypeptides; salt-forming counterions; antiseptics such as benzalkonium chloride, benzoic acid, salicylic acid, thimerosal, phenethyl alcohol, methylparaben, propylparaben, chlorhexidine, sorbic acid, and hydrogen peroxide; solvents such as glycerin, propylene glycol, and polyethylene glycol; sugar alcohols such as mannitol and sorbitol; suspending agents; surfactants such as PEG, sorbitan ester, polysorbates such as polysorbate 20 and polysorbate 80, triton, tromethamine, lecithin, and cholesterol; stability enhancers such as sucrose and sorbitol; elasticity enhancers such as sodium chloride, potassium chloride, mannitol, and sorbitol; transport agents; diluents; excipients; and/or pharmaceutical additives.

The amount of these pharmaceutical materials added is 0.001 to 1000 times, preferably 0.01 to 100 times, more preferably 0.1 to 10 times the weight of the anti-GPRC5D antibody or antigen-binding fragment thereof, and/or the molecule comprising at least one of them.

An immunoliposome comprising the anti-GPRC5D antibody or antigen-binding fragment thereof, and/or the molecule comprising at least one of them, encapsulated in a liposome, or a pharmaceutical composition comprising a modified antibody form comprising the antibody conjugated with a liposome (U.S. Pat. No. 6,214,388, etc.) is also included in the pharmaceutical composition of the present invention.

The excipients or vehicles are not particularly limited as long as they are liquid or solid materials usually used in injectable water, saline, artificial cerebrospinal fluids, and other preparations for oral or parenteral administration. Examples of saline can include neutral saline and serum albumin-containing saline.

Examples of buffers can include a Tris buffer adjusted to bring the final pH of the pharmaceutical composition to 7.0 to 8.5, an acetate buffer adjusted to bring the final pH thereof to 4.0 to 5.5, a citrate buffer adjusted to bring the final pH thereof to 5.0 to 8.0, and a histidine buffer adjusted to bring the final pH thereof to 5.0 to 8.0.

The pharmaceutical composition of the present invention is a solid, a liquid, a suspension, or the like. Another example of the pharmaceutical composition of the present invention can include freeze-dried preparations. The freeze-dried preparations can be formed using an excipient such as sucrose.

The administration route of the pharmaceutical composition of the present invention may be any of enteral administration, local administration, and parenteral administration. Examples thereof can include intravenous administration, intraarterial administration, intramuscular administration, intradermal administration, hypodermic administration, intraperitoneal administration, transdermal administration, intraosseous administration, and intraarticular administration.

The composition of such a pharmaceutical composition can be determined according to the administration method, the binding affinity of the antibody for the GPRC5D protein, etc.

The dose of the pharmaceutical composition of the present invention is not limited as long as the dose is a pharmacologically effective amount. The dose can be appropriately determined according to the species of an individual, the type of disease, symptoms, sex, age, pre-existing conditions, the binding affinity of the antibody for the GPRC5D protein or its biological activity, and other factors. A dose of usually 0.01 to 1000 mg/kg, preferably 0.1 to 100 mg/kg, can be administered once every day to every 180 days or twice or three or more times a day.

Examples of the form of the pharmaceutical composition can include injections (including freeze-dried preparations and drops), suppositories, transnasal absorption preparations, transdermal absorption preparations, sublingual formulations, capsules, tablets, ointments, granules, aerosols, pills, powders, suspensions, emulsions, eye drops, and biological implant formulations.

The pharmaceutical composition of the present invention can be administered concurrently with or separately from an additional drug. For example, the pharmaceutical composition of the present invention may be administered after administration of the additional drug, or the additional drug may be administered after administration of the pharmaceutical composition. Alternatively, the pharmaceutical composition and the additional drug may be administered concurrently. Examples of the additional drug can include various anticancer agents such as chemotherapeutics and radiation therapeutics. These cases are collectively referred to as the “combined use with an additional drug” of the antibody of the present invention. A pharmaceutical composition comprising the active ingredient of the pharmaceutical composition of the present invention as well as an additional drug is also included in the present invention.

The present invention provides a method for treating or preventing GPRC5D-related diseases such as cancer, use of the antibody of the present invention for preparing a pharmaceutical composition for treatment or prevention of the diseases, and use of the antibody of the present invention for treating or preventing the diseases. The present invention also encompasses a kit for treatment or prevention comprising the antibody of the present invention.

### EXAMPLES

Hereinafter, the present invention will be further specifically described with reference to the Examples. However, the present invention is not intended to be limited to them.

Procedures related to gene manipulation in the Examples below were performed according to the methods described in “Molecular Cloning” (Sambrook, J., Fritsch, E. F. and Maniatis, T., Cold Spring Harbor Laboratory Press, 1989) or the methods described in other experimental manuals used by those skilled in the art, or using commercially available reagents or kits according to the instruction manuals, unless otherwise specified.

### Example 1

### Preparation of Rat Anti-Human GPRC5D Antibody

### 1)-1 Immunization Using Human GPRC5D Expression Vector

1)-1-1 Construction of Human GPRC5D Expression Vector (pcDNA3.1-DEST-hGPRC5D)

pcDNA3.1-DEST engineered as a destination vector was prepared from pcDNA3.1(+) using Gateway Vector Convension System (Thermo Fisher Scientific Inc.). A cDNA encoding the human GPRC5D protein (NP_061124.1) was cloned into the pcDNA3.1-DEST vector using Gateway LR Clonase Enzyme mix (Life Technologies Corp.) to construct a human GPRC5D expression vector pcDNA3.1-DEST-hGPRC5D. For the large-scale preparation of the human GPRC5D expression vector, Endofree Plasmid Giga Kit (Qiagen N.V.) was used.

### 1)-1-2 Rat Immunization

For immunization, WKY/lzm female rats (Japan SLC, Inc.) were used. First, both lower thighs of each rat were pretreated with hyaluronidase (Sigma-Aldrich Corp.). Then, the pcDNA3.1-DEST-hGPRC5D was intramuscularly injected to these sites. Subsequently, the in vivo electroporation of these sites was carried out using ECM830 (BTX) and a needle electrode. The same in vivo electroporation as above was repeated once per approximately two weeks. Then, the lymph node or the spleen was collected from the rat and used in hybridoma preparation.

### 1)-2 Hybridoma Preparation

The lymph node cells or the spleen cells were electrically fused with mouse myeloma SP2/0-agl4 cells (ATCC, No. CRL-1 581) using LF301 Cell Fusion Unit (BEX Co., Ltd.). The fused cells were diluted with ClonaCell-HY Selection Medium D (StemCell Technologies Inc.) and cultured. Hybridoma colonies that appeared were recovered to thereby prepare monoclonal hybridomas. Each hybridoma colony thus recovered was cultured using ClonaCell-HY Selection Medium E (StemCell Technologies Inc.), and the obtained hybridoma culture supernatant was used to screen for an anti-human GPRC5D antibody-producing hybridoma.

### 1)-3 Antibody Screening by Cell-ELISA

### 1)-3-1 Primary Screening by Cell-ELISA

Cell line HEK293a cells (HEK293 cells stably transfected with integrin αv and integrin β3 expression vectors) were adjusted to 5×10³ cells/mL in a DMEM medium containing 10% FBS. pcDNA3.1-DEST-hGPRC5D or a control pcDNA3.1-DEST was transfected thereto according to transfection procedures using Lipofectamine 2000 (Thermo Fisher Scientific Inc.). The resulting cells were dispensed in an amount of 100 μL/well to a 96-well plate (Corning Inc.) and cultured overnight at 37° C. under 5% CO₂ conditions in a DMEM medium containing 10% FBS. The obtained transfected cells were used in the attached state in Cell-ELISA.

### 1)-3-2 Cell-ELISA

After removal of the culture supernatant from the expression vector-transfected HEK293a cells prepared in Example 1)-1-1, each hybridoma culture supernatant was added to the pcDNA3.1-DEST-hGPRC5D, or pcDNA3.1-DEST-transfected HEK293a cells, and the plate was left standing at 4° C. for 1 hour. The cells in the wells were washed once with PBS containing 5% FBS. Then, Anti-Rat IgG, HRP-Linked Whole Ab Goat (GE Healthcare Bio-Sciences Corp.) diluted 500-fold with PBS containing 5% FBS was added thereto, and the plate was left standing at 4° C. for 1 hour. The cells in the wells were washed twice with PBS containing 5% FBS. Then, an OPD chromogenic solution (OPD solution (o-phenylenediamine dihydrochloride (Wako Pure Chemicals Industries, Ltd.) and H₂O₂ dissolved at concentrations of 0.4 mg/mL and 0.6% (v/v), respectively, in 0.05 M trisodium citrate and 0.1 M disodium hydrogen phosphate dodecahydrate, pH 4.5)) was added thereto at a concentration of 100 μL/well. Color reaction was performed with occasional stirring and stopped by the addition of 1 M HCL at a concentration of 100 μL/well. Then, the absorbance was measured at 490 nm using a plate reader (ENVISION; PerkinElmer, Inc.). In order to select a hybridoma producing an antibody specifically binding to human GPRC5D expressed on cell membrane surface, hybridomas that yielded a culture supernatant exhibiting higher absorbance for the pcDNA3.1-DEST-hGPRC5D expression vector-transfected HEK293a cells compared with the pcDNA3.1-DEST-transfected HEK293a cells of control were selected as anti-human GPRC5D antibody production-positive hybridomas.

### 1)-4 Screening Antibody by Flow Cytometry

### 1)-4-1 Preparation of Antigen Gene-Expressing Cell for Flow Cytometry Analysis

HEK293T cells (Thermo Fisher Scientific Inc.) were inoculated at a concentration of 4×10³ cells/mL to a 225-cm² flask and cultured overnight at 37° C. under 5% CO₂ conditions in a DMEM medium containing 10% FBS. On the next day, pcDNA3.1-DEST-hGPRC5D or a control pcDNA3.1-DEST was transfected to the HEK293T cells using Lipofectamine LTX (Thermo Fisher Scientific Inc.), and the cells were further cultured overnight at 37° C. under 5% CO₂ conditions. On the next day, the expression vector-transfected HEK293T cells were treated with TrypLE Express (Thermo Fisher Scientific Inc.), washed with DMEM containing 10% FBS, and then adjusted to a concentration of 5×10⁶ cells/mL in PBS containing 5% FBS. The obtained cell suspension was used in flow cytometry analysis.

1)-4-2 Flow Cytometry Analysis

The human GPRC5D binding specificity of the antibody produced by each hybridoma determined to be positive by Cell-ELISA in Example 1)-3 was further confirmed by flow cytometry. Each HEK293T cell suspension prepared in Example 1)-4-1 was inoculated at a concentration of 100 μL/well to a 96-well U-bottomed microplate and centrifuged to remove a supernatant. The pcDNA3.1-DEST-hGPRC5D transfected HEK293T cells or the pcDNA3.1-DEST-transfected HEK293T cells were suspended by the addition of the hybridoma culture supernatant and left standing at 4° C. for 1 hour. The cells were washed once with PBS containing 5% FBS, then suspended by the addition of PE Goat Anti-Rat Ab (BD) diluted 100-fold with PBS containing 5% FBS, and left standing at 4° C. for 30 minutes. The cells were washed twice with PBS containing 5% FBS and then resuspended in PBS containing 5% FBS, followed by detection using a flow cytometer (FACSCanto™ II:BD). The data was analyzed using Flowjo (Tree Star, Inc.). The PE fluorescence intensity of the cell fraction was plotted to a histogram. Hybridomas that yielded a sample exhibiting a shift to stronger fluorescence intensity in the histogram of the pcDNA3.1-DEST-hGPRC5D transfected HEK293T cells compared with the fluorescence intensity histogram of the control pcDNA3.1-DEST-transfected HEK293T cells were selected as hybridomas producing the anti-human GPRC5D antibody.

### 1)-5 Antibody Screening by ADCC Assay

### 1)-5-1 Preparation of Cell Stably Expressing β-Galactosidase

HEK293FT cells (Thermo Fisher Scientific Inc.) were transfected with pLenti6/V5-GW/LacZ and ViraPower™ Packaging Mix (Thermo Fisher Scientific Inc.) according to the attached protocols to prepare a recombinant lentivirus expressing the β-galactosidase gene. HEK293T cells were infected by the obtained recombinant lentivirus according to the protocol of ViraPower Lentiviral Expression Systems (Thermo Fisher Scientific Inc.). Virus-infected cells were selected using 10 μg/mL

Blasticidin (Thermo Fisher Scientific Inc.) to obtain a line stably expressing β-galactosidase. These HEK293T cells stably expressing β-galactosidase (hereinafter, referred to as “293T-lacZ cells”) were used as target cells in the assay of ADCC activity.

### 1)-5-2 Preparation of Target Cell

The 293T-lacZ cells obtained in Example 1)-5-1 were inoculated at a concentration of 5×10³ cells/ml to a 75-cm² flask and cultured overnight at 37° C. under 5% CO₂ conditions in a DMEM medium containing 10% FBS. On the next day, pcDNA3.1-DEST-hGPRC5D or a control pcDNA3.1-DEST was transfected to the 293T-lacZ cells using Lipofectamine LTX (Thermo Fisher Scientific Inc.), and the cells were further cultured overnight at 37° C. under 5% CO₂ conditions. On the next day, the expression vector-transfected 293T-lacZ cells were treated with TrypLE Express (Thermo Fisher Scientific Inc.) and washed twice with a phenol red-free RPMI1640 medium containing 5% FBS (Thermo Fisher Scientific Inc.) (hereinafter, referred to as a “medium for ADCC”). The number of live cells was counted by the trypan blue dye exclusion test. The cells were resuspended to 1×10³ cells/mL in a medium for ADCC and used as target cells.

### 1)-5-3 Preparation of Effector Cell

Human peripheral blood mononuclear cells (PBMC) collected from the blood of a volunteer according to the standard method using Ficoll-Paque PLUS (GE Healthcare Bio-Sciences Corp.) were suspended in a phenol red-free RPMI1640 medium containing 10% FBS (Thermo Fisher Scientific Inc.), centrifuged, and then resuspended. The number of live cells was counted by the trypan blue dye exclusion test. After centrifugation, the medium was removed, and the cells were suspended and adjusted to 2×10⁶ cells/mL in a medium for ADCC and used as effector cells.

### 1)-5-4 Preparation of Hybridoma Culture Supernatant

The concentration of each rat anti-GPRC5D antibody-producing hybridoma culture supernatant obtained in Example 1)-4 was measured using FastELISA IgG ELISA Quantification Kit (RD-Biotech) and adjusted to 10 μg/mL (final concentration) with ClonaCell-HY Selection Medium E (StemCell Technologies Inc.).

### 1)-5-5 ADCC Assay

The 293T-lacZ cells obtained in Example 1)-5-2 were added at a concentration of 50 μl/well to a 96-well U-bottomed microplate. Each hybridoma culture supernatant prepared in Example 1)-5-4, or a mouse anti-GPRC5D IgG2b antibody (R&D Systems, Inc.) for a positive control or a mouse control antibody (mIgG2b) (R&D Systems, Inc.) for a negative control adjusted to 10 μg/mL (final concentration) was added thereto at a concentration of 50 μl/well, and the plate was left standing at 4° C. for 1 hour. The effector cells prepared in Example 1)-5-3 were further added thereto at a concentration of 50 μl/well. The plate was centrifuged at 1200 rpm at room temperature for 5 minutes, followed by culture at 37° C. for 18 hours under 5% CO₂ conditions. 50 μl of the supernatant in each well was recovered into a white plate (Corning Inc.). A solution of β-Glo assay system (Promega Corp.) was added thereto in an amount of 50 μl/well. The luminescence intensity was measured using a plate reader (ENVISION; PerkinElmer, Inc.). The percentage of cells lysed by ADCC activity was calculated according to the following expression:

Percentage of cells lysed (%)=(A−B)/C−B)×100

A: Count of sample well

B: Average of spontaneous release (wells supplemented with neither the antibody nor the effector cells) counts (n=3). The same operation as in the sample well was performed except that 50 μL of a medium for ADCC were added instead of the hybridoma culture supernatant and the effector cells.

C: Average of maximum release (wells containing target cells lysed in a surfactant) counts (n=3). 50 μl of a medium for ADCC were added instead of the hybridoma culture supernatant and the effector cells. For the assay, 150 μL of the β-Glo assay system solution was added to each well containing the cells and mixed therewith. A 100 μL aliquot thereof was added to a white plate to carry out the assay.

The ADCC activity against the pcDNA3.1-DEST-hGPRC5D-transfected 293T-lacZ cells or the pcDNA3.1-DEST-transfected 293T-lacZ cells was calculated according to the method described above to select hybridoma clones that exhibited ADCC activity specific for the pcDNA3.1-DEST-hGPRC5D-transfected 293T-lacZ cells and produced an antibody exhibiting ADCC activity higher than that of the positive control antibody.

### 1)-6 Isotyping of Antibody

2A4, 2B1 and 7B4 suggestive of strongly binding to human GPRC5D and having high ADCC activity in Example 1)-5 were selected from among the rat anti-GPRC5D antibody-producing hybridomas obtained in Example 1)-4, and identified by antibody isotyping. The isotypes were determined using Rat monoclonal isotyping test kit (AbD Serotec). As a result, all the isotypes of the rat anti-GPRC5D monoclonal antibodies 2A4, 2B1, and 7B4 were confirmed to be IgG2b and κ chains.

### 1)-7 Preparation of Monoclonal Antibody

The rat anti-GPRC5D monoclonal antibody was purified from the hybridoma culture supernatant. First, the 2A4, 2B1 and 7B4-producing hybridoma was allowed to grow into a sufficient amount in ClonaCell-HY Selection Medium E (StemCell Technologies Inc.). Then, the medium was replaced with a Hybridoma SFM (Thermo Fisher Scientific Inc.) containing 5 μg/mL gentamicin (Thermo Fisher Scientific Inc.) supplemented with 20% Ultra Low IgG FBS (Thermo Fisher Scientific Inc.), followed by culture for 5 days. This culture supernatant was recovered and sterilized through a 0.45 μm filter.

Each antibody was purified from the hybridoma supernatant in one step by protein G affinity chromatography (at 4 to 6° C.). A buffer replacement step after the protein G affinity chromatography purification was carried out at 4 to 6° C. First, the hybridoma culture supernatant was applied to a column packed with protein G (GE Healthcare Bio-Sciences Corp.) equilibrated with PBS. After entry of the whole culture supernatant solution into the column, the column was washed with PBS in an amount of twice or more the column volume. Next, antibody-containing fractions were collected by elution with an aqueous solution of 0.1 M glycine/hydrochloric acid (pH 2.7). The collected fractions were adjusted to pH 7.0 to 7.5 by the addition of 1 M Tris-HCl (pH 9.0). Then, the buffer was replaced with HBSor (25 mM histidine/5% sorbitol, pH 6.0) using Centrifugal UF Filter Device VIVASPIN20 (molecular weight cutoff: UF30K, Sartorius Japan K.K., at 4 to 6° C.) while the antibody was concentrated and adjusted to an antibody concentration of 1 mg/mL or higher. Finally, the concentrate was filtered through Minisart-Plus filter (Sartorius Japan K.K.) and used as a purified sample.

### Example 2

### In Vitro Evaluation of Rat Anti-GPRC5D Antibodies (2A4, 2B1, and 7B4)

### 2)-1 Study on Binding Activity of Obtained Rat Anti-GPRC5D Antibodies (2A4, 2B1, and 7B4) Against Human GPRC5D by Flow Cytometry

Human multiple myeloma cell line KHM-1B cells (JCRB Cell Bank) expressing GPRC5D were adjusted to a concentration of 5×10⁶ cells/mL with PBS containing 5% FBS, inoculated in an amount of 100 μL/well to a 96-well U-bottomed microplate, and centrifuged to remove a supernatant. Each rat anti-GPRC5D antibody (2A4, 2B1, and 7B4) adjusted to 0.32 ng/mL to 10 μg/mL in Example 1)-7 was added thereto in an amount of 100 μL/well, and the plate was left standing at 4° C. for 1 hour. The cells were washed twice with PBS containing 5% FBS. Then, PE Goat Anti-Rat Ab (Becton, Dickinson and Company) diluted 100-fold with PBS containing 5% FBS was added thereto at a concentration of 100 μL/well, and the plate was left standing at 4° C. for 1 hour. The cells were washed twice with PBS containing 5% FBS and then resuspended in PBS containing 5% FBS, followed by detection using a flow cytometer (FACSCanto™ II; Becton, Dickinson and Company). The data was analyzed using Flowjo (Tree Star, Inc.). The PE fluorescence intensity of the cell fraction was plotted to a histogram, and the mean fluorescence intensity (MFI) was calculated. As shown in FIG. 1, 2A4, 2B1, and 7B4 were found to bind to human GPRC5D (2A4 MFI (max): 1153, 2A4 Kd: 0.5 nM, 2B1 MFI (max): 2386, 2B1 Kd: 14.8 nM, 7B4 MFI (max): 2492, 7B4 Kd: 1.3 nM). Flow cytometry was also carried out using human multiple myeloma cell line KMS-34 cells (JCRB Cell Bank) expressing GPRC5D and produced similar results (2A4 MFI (max): 2064, 2A4 Kd: 1.4 nM, 2B1 MFI (max): 3157, 2B1 Kd: 12.7 nM, 7B4 MFI (max): 4471, 7B4 Kd: 2.1 nM).

### 2)-2 Identification of Epitope for Obtained Rat Anti-GPRC5D Antibodies (2A4, 2B1, and 7B4)

### 2)-2-1 Identification of Epitope by ELISA

Epitopes bound by the obtained rat anti-GPRC5D antibodies (2A4, 2B1, and 7B4) were identified using a human GPRC5D amino-terminal peptide MYKDCIESTGDYFLLCDAEGPWGIILE(Biotin)-NH₂ (Sigma-Aldrich Corp.) (SEQ ID NO: 1 of the Sequence Listing; FIG. 2) having a biotinylated carboxy-terminal peptide and lacking an intramolecularly formed disulfide bond, and a human GPRC5D amino-terminal peptide MYKDCIESTGDYFLLCDAEGPWGIILE-K(Biotin)-NH₂ (Peptide Institute, Inc.) (SEQ ID NO: 2 of the Sequence Listing; FIG. 3) having a lysine-containing biotinylated carboxy terminus and having an intramolecularly formed disulfide bond. Each peptide diluted with PBS was added to Nunc Immobilizer (Thermo Fisher Scientific Inc.), and the plate was left standing at room temperature for 1 hour. After washing three times with PBST, FBS containing 1% BSA was added thereto, and the plate was left standing at room temperature for 1 hour. After washing three times with PBST, each rat anti-GPRC5D antibody (2A4, 2B1, and 7B4) prepared in Example 1)-7 and diluted to 0.1 ng/mL to 1 μg/mL with PBS was added thereto, and the plate was left standing at room temperature for 1 hour. After washing three times with PBST, Anti-Rat IgG, HRP-Linked Whole Ab Goat (GE Healthcare Bio-Sciences Corp.) diluted 500-fold with PBS was added thereto, and the plate was left standing at room temperature for 1 hour. After washing three times with PBST, SuperSignal™ ELISA Pico Chemiluminescent Substrate (Thermo Fisher Scientific Inc.) was added thereto, and the luminescence was measured using a plate reader (ENVISION; PerkinElmer, Inc.). As a result, the 2B1 antibody bound to the amino-terminal peptide sequence of human GPRC5D whereas the 2A4 and 7B4 antibodies did not bind thereto, regardless of the presence or absence of a disulfide bond. These results suggested that the epitope for the 2B1 antibody was present in the amino-terminal region of human GPRC5D while the epitopes for the 2A4 and 7B4 antibodies were present in regions other than the amino terminus.

### 2)-2-2 Identification of Epitope by Flow Cytometry

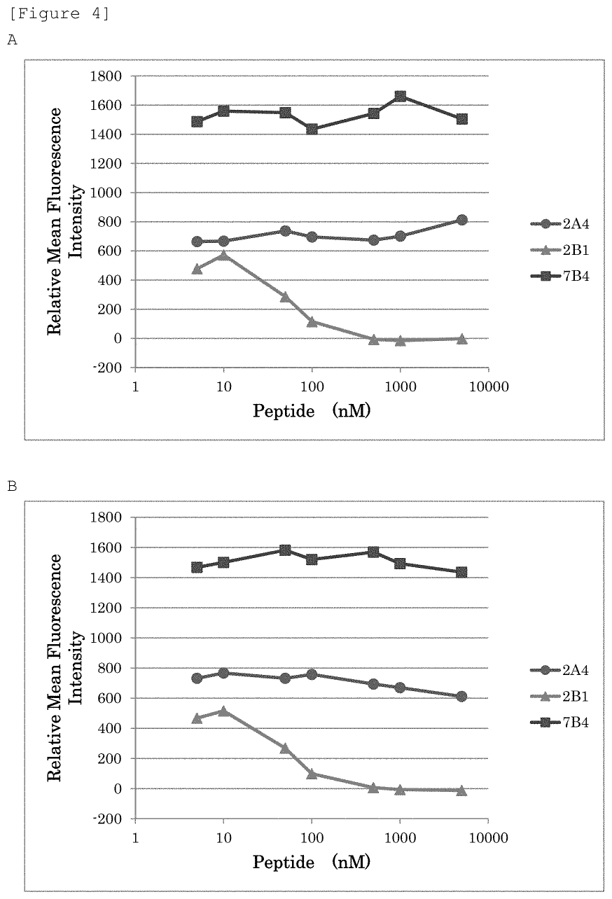

Human multiple myeloma cell line KMS-34 cells expressing GPRC5D were adjusted to a concentration of 2×10⁶ cells/mL with PBS containing 5% FBS, inoculated in an amount of 100 μL/well to a 96-well U-bottomed microplate, and centrifuged to remove a supernatant. Each rat anti-GPRC5D antibody (2A4, 2B1, and 7B4) adjusted to 7.5 μg/mL in Example 1)-7, or Rat IgG2b isotype control antibody (Medical & Biological Laboratories Co., Ltd. (MBL)) was added thereto in an amount of 100 μL/well. The two types of peptides used in Example 2)-2-1 were each adjusted to 17 ng/mL to 34 μg/mL with PBS and added in an amount of 100 μL/well to the wells containing the antibody dilution, and the plate was left standing at 4° C. for 1 hour. The cells were washed twice with PBS containing 5% FBS. Then, PE Goat Anti-Rat Ab (Becton, Dickinson and Company) diluted 100-fold with PBS containing 5% FBS was added thereto in an amount of 100 μL/well, and the plate was left standing at 4° C. for 1 hour. The cells were washed twice with PBS containing 5% FBS and then resuspended in PBS containing 5% FBS, followed by detection using a flow cytometer (FACSCanto™ II; Becton, Dickinson and Company). The data was analyzed using Flowjo (Tree Star, Inc.). The PE fluorescence intensity of the cell fraction was plotted to a histogram, and the mean fluorescence intensity (MFI) was calculated. The MFI value of the control antibody was subtracted from the MFI value of the GPRC5D antibody to calculate a relative value of MFI (rMFI). As shown in FIG. 4 (intramolecular disulfide bond was present (A) or absent (B)), the binding of 2B1 was found to be inhibited by the addition of the amino-terminal peptide of human GPRC5D, regardless of the presence or absence of a disulfide bond. By contrast, the binding of the 2A4 and 7B4 antibodies was found to be not inhibited by the addition of the amino-terminal peptide of human GPRC5D. These results suggested that the epitope for the 2B1 antibody was present in the amino-terminal region of human GPRC5D while the epitopes for the 2A4 and 7B4 antibodies were present in regions other than the amino terminus.

### 2)-3 ADCC Activity Evaluation of Obtained Rat Anti-GPRC5D Antibodies (2A4, 2B1, and 7B4)

### 2)-3-1 Preparation of Target Cell

Human multiple myeloma cell line KHM-1B cells were adjusted to a concentration of 2×10⁶ cells/mL with an RPMI1640 medium containing 10% FBS (Thermo Fisher Scientific Inc.). 100 μL of Chromium-51 Radionuclide (PerkinElmer, Inc.) was added per mL of the cell suspension, and the cells were cultured at 37° C. for 2 hours under 5% CO₂ conditions. The cells were washed three times with an RPMI1640 medium containing 10% FBS, then resuspended to 2×10⁵ cells/mL in an RPMI1640 medium containing 10% FBS, and used as target cells.

### 2)-3-2 Preparation of Effector Cell

PBMC prepared in Example 1)-5-3 was differentiated into NK cells using BINKIT (Biotherapy Institute of Japan). The NK cells were adjusted to 1×10⁶ cells/mL with an RPMI1640 medium containing 10% FBS and used as effector cells.

### 2)-3-3 ADCC Assay

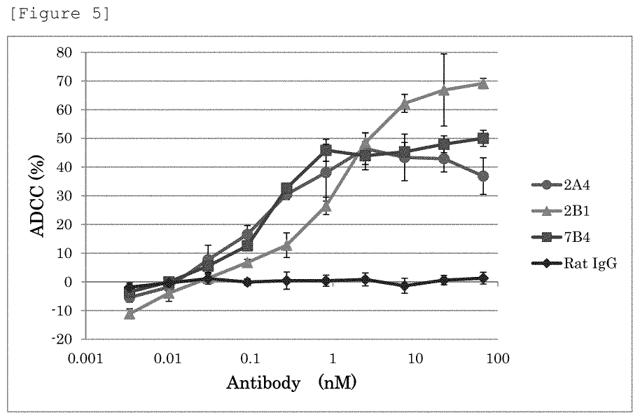

The KHM-1B cells prepared in Example 2)-3-1 were added at a concentration of 50 μL/well to a 96-well U-bottomed microplate. Each obtained rat anti-GPRC5D antibody (2A4, 2B1, and 7B4) or a rat control antibody (rIgG2b) adjusted to 0.5081 ng/mL to 10 μg/mL (final concentration) was added thereto in an amount of 50 μL/well, and the plate was left standing at 4° C. for 30 minutes. The effector cells prepared in Example 2)-3-2 were further added thereto in an amount of 100 μL/well. After centrifugation at room temperature at 1200 rpm for 3 minutes, the cells were cultured at 37° C. for 4 hours under 5% CO₂ conditions. A 50 μL aliquot of the supernatant was recovered into LumaPlate (PerkinElmer, Inc.) and dried overnight at 50° C., followed by measurement using a plate reader (TopCount; PerkinElmer, Inc.). The percentage of cells lysed by ADCC activity was calculated according to Example 1)-5-5. As shown in FIG. 5, 2A4, 2B1, and 7B4 were found to have ADCC activity.

### Example 3

### Sequencing of cDNAs Encoding Variable Regions of Rat Anti-GPRC5D Antibodies (2A4, 2B1, and 7B4)

3)-1 Sequencing of cDNAs Encoding Variable Regions of 2A4

### 3)-1-1 Preparation of Total RNA From 2A4-Producing Hybridoma

In order to amplify the cDNAs encoding the variable regions of 2A4, total RNA was prepared from the 2A4-producing hybridoma using TRIzol Reagent (Ambion/Thermo Fisher Scientific Inc.).

3)-1-2 Synthesis of cDNA (5′-RACE-Ready cDNA)

cDNAs (5′-RACE-Ready cDNAs) were synthesized using approximately 1 μg of the total RNA prepared in Example 3)-1-1, and SMARTer RACE cDNA Amplification Kit (Clontech Laboratories, Inc.).

3)-1-3 Amplification and Sequencing of cDNA Encoding Heavy Chain Variable Region of 2A4 by 5′-RACE PCR

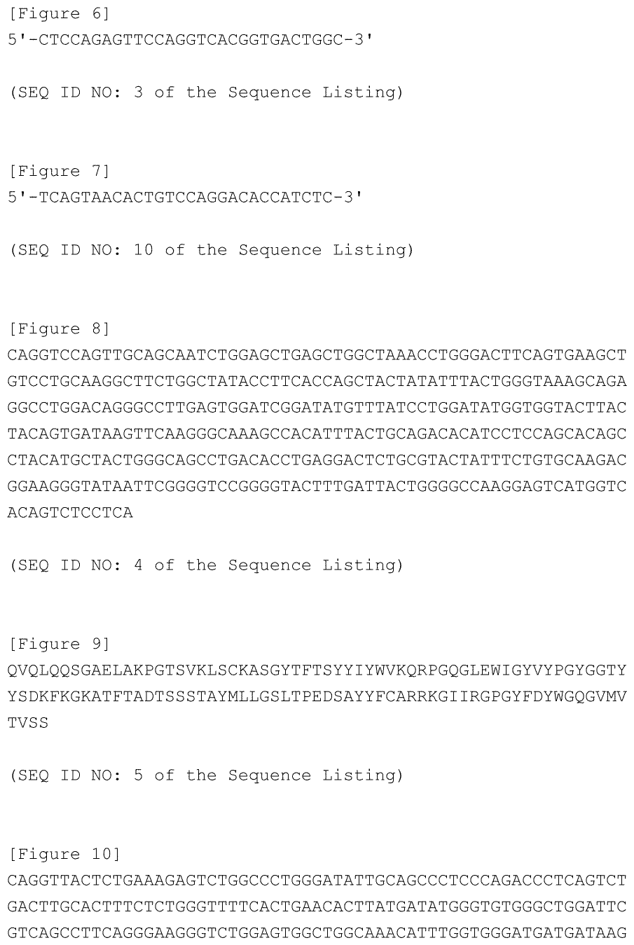

The primers used for the PCR amplification of the variable region-encoding cDNA of the heavy chain gene of 2A4 were oligonucleotides having the sequences of UPM (Universal Primer A Mix; attached to SMARTer RACE cDNA Amplification Kit) and 5′-CTCCAGAGTTCCAGGTCACGGTGACTGGC-3′ (RG2AR3: SEQ ID NO: (FIG. 6)). The UPM used was attached to SMARTer RACE cDNA Amplification Kit (Clontech Laboratories, Inc.), while RG2AR3 was designed from the sequences of rat heavy chain constant regions registered in the database.

The cDNA encoding the heavy chain variable region of 2A4 was amplified by 5′-RACE PCR using this primer set and the cDNAs (5′-RACE-Ready cDNAs) synthesized in Example 3)-1-2 as templates. This PCR was carried out on the Touchdown PCR program according to the manual of SMARTer RACE cDNA Amplification Kit (Clontech Laboratories, Inc.).

The heavy chain variable region-encoding cDNA amplified by 5′-RACE PCR was purified using MinElute PCR Purification Kit (Qiagen N.V.) and then cloned using Zero Blunt TOPO PCR Cloning Kit (Invitrogen Corp.). The nucleotide sequence of the cloned heavy chain variable region-encoding cDNA was subjected to sequence analysis.

The determined nucleotide sequence of the cDNA encoding the heavy chain variable region of 2A4 is shown in SEQ ID NO: 4 (FIG. 8), and the amino acid sequence thereof is shown in SEQ ID NO: 5 (FIG. 9).

3)-1-4 Amplification and Sequencing of cDNA Encoding Light Chain Variable Region of 2A4 by 5′-RACE PCR

The primers used for the PCR amplification of the variable region-encoding cDNA of the light chain gene of 2A4 were oligonucleotides having the sequences of UPM (Universal Primer A Mix; attached to SMARTer RACE cDNA Amplification Kit) and 5′-TCAGTAACACTGTCCAGGACACCATCTC-3′ (RKRS: SEQ ID NO: 10 (FIG. 7)). The UPM used was attached to SMARTer RACE cDNA Amplification Kit (Clontech Laboratories, Inc.), while RKRS was designed from the sequences of rat light chain constant regions registered in the database.

The cDNA encoding the light chain variable region of 2A4 was amplified by 5′-RACE PCR using this primer set and the cDNAs (5′-RACE-Ready cDNAs) synthesized in Example 3)-1-2 as templates. This PCR was carried out on the Touchdown PCR program according to the manual of SMARTer RACE cDNA Amplification Kit (Clontech Laboratories, Inc.).

The light chain variable region-encoding cDNA amplified by 5′-RACE PCR was purified using MinElute PCR Purification Kit (Qiagen N.V.) and then cloned using Zero Blunt TOPO PCR Cloning Kit (Invitrogen Corp.). The nucleotide sequence of the cloned light chain variable region-encoding cDNA was subjected to sequence analysis.

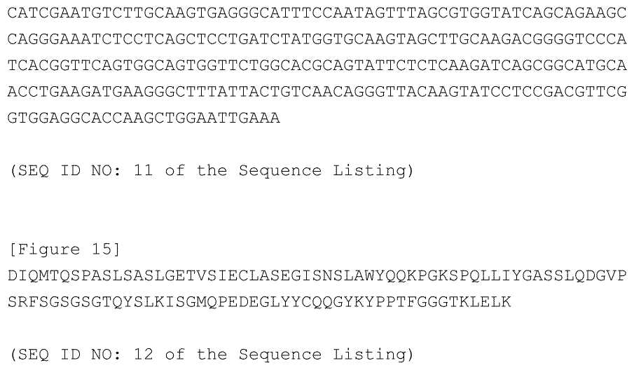

The determined nucleotide sequence of the cDNA encoding the light chain variable region of 2A4 is shown in SEQ ID NO: 11 (FIG. 14), and the amino acid sequence thereof is shown in SEQ ID NO: 12 (FIG. 15).

3)-2 Sequencing of cDNAs Encoding Variable Regions of 2B1

Sequences were determined in the same way as in Example 3)-1.

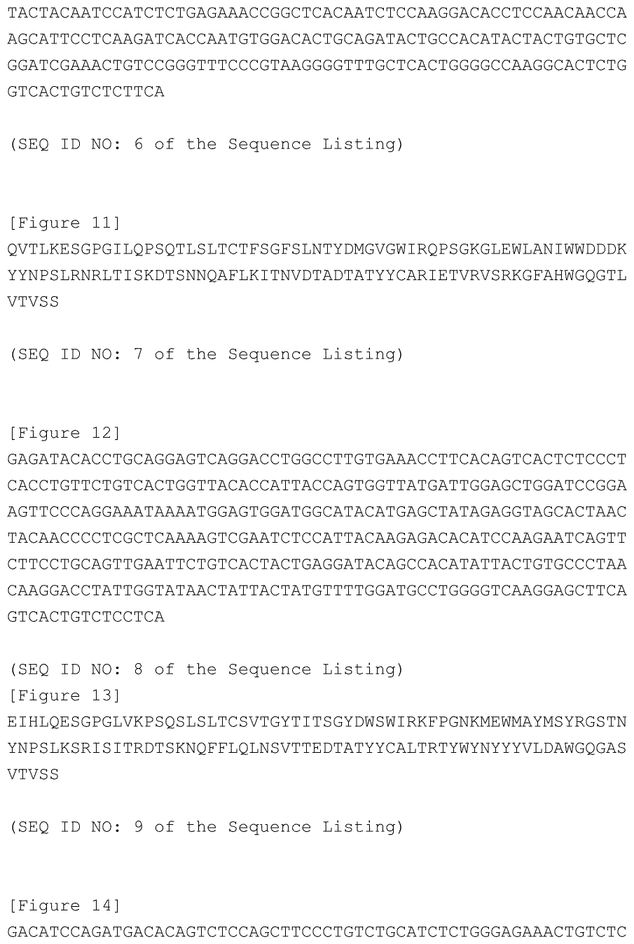

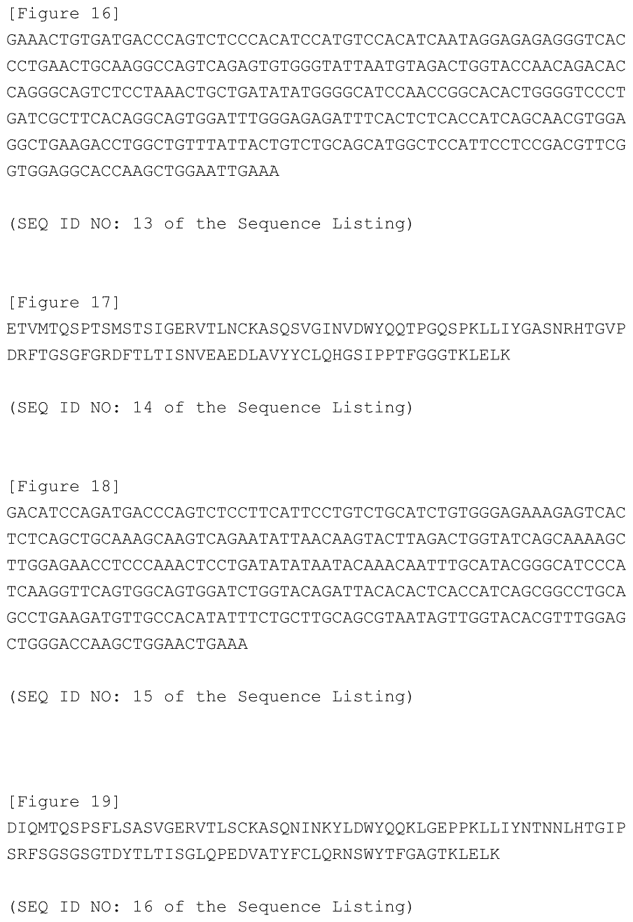

The determined nucleotide sequence of the cDNA encoding the heavy chain variable region of 2B1 is shown in SEQ ID NO: 6 (FIG. 10), and the amino acid sequence thereof is shown in SEQ ID NO: 7 (FIG. 11). The nucleotide sequence of the cDNA encoding the light chain variable region is shown in SEQ ID NO: 13 (FIG. 16), and the amino acid sequence thereof is shown in SEQ ID NO: 14 (FIG. 17).

3)-3 Sequencing of cDNAs Encoding Variable Regions of 7B4

Sequences were determined in the same way as in Example 3)-1.

The determined nucleotide sequence of the cDNA encoding the heavy chain variable region of 7B4 is shown in SEQ ID NO: 8 (FIG. 12), and the amino acid sequence thereof is shown in SEQ ID NO: 9 (FIG. 13). The nucleotide sequence of the cDNA encoding the light chain variable region is shown in SEQ ID NO: 15 (FIG. 18), and the amino acid sequence thereof is shown in SEQ ID NO: 16 (FIG. 19).

### Example 4

### Preparation of Human Chimeric Anti-GPRC5D Antibodies (c2A4, c2B1, and c7B4)

4)-1 Construction of Chimeric and Humanized Light Chain Expression Vector pCMA-LK

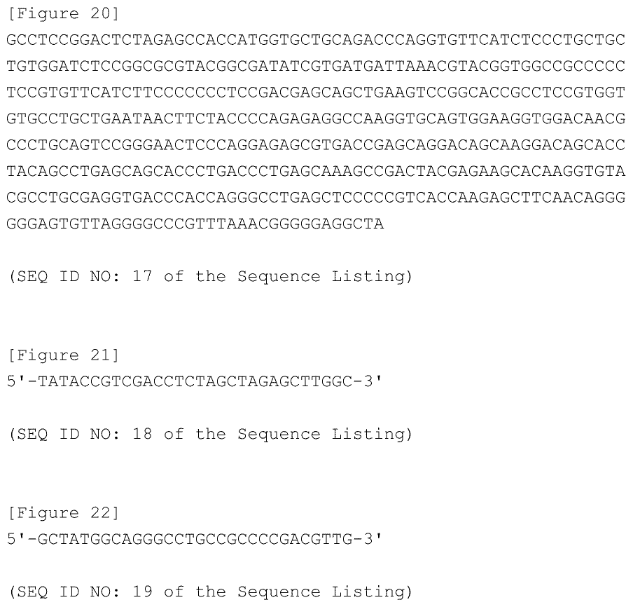

A plasmid pcDNA3.3-TOPO/LacZ (Invitrogen Corp.) was digested with restriction enzymes XbaI and PmeI. The obtained fragment of approximately 5.4 kb was fused with a DNA fragment comprising a DNA sequence (shown in SEQ ID NO: 17 (FIG. 20) of the Sequence Listing) encoding a human light chain secretory signal and a human κ chain constant region using an In-Fusion Advantage PCR cloning kit (Clontech Laboratories, Inc.) to prepare pcDNA3.3/LK.

PCR was performed with pcDNA3.3/LK as a template using a primer set shown below. The obtained fragment of approximately 3.8 kb was phosphorylated and then self-ligated to construct a chimeric and humanized light chain expression vector pCMA-LK having the nucleotide sequence encoding a signal sequence, a cloning site, and the human κ chain constant region, downstream of the CMV promoter.

### Primer Set

5′-TATACCGTCGACCTCTAGCTAGAGCTTGGC-3′
(3.3-F1: SEQ ID NO: 18 of the Sequence Listing;
FIG. 21)
5′-GCTATGGCAGGGCCTGCCGCCCCGACGTTG-3′
(3.3-R1: SEQ ID NO: 19 of the Sequence Listing;
FIG. 22)

4)-2 Construction of Chimeric and Humanized IgG1 Type Heavy Chain Expression Vector pCMA-G1

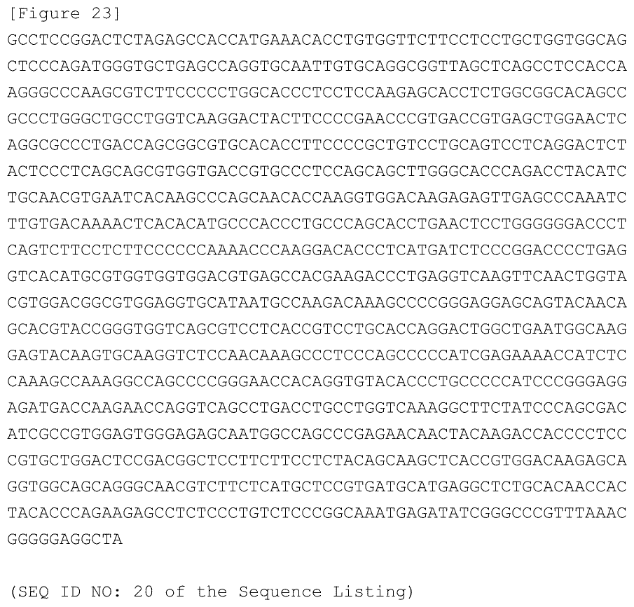

pCMA-LK was digested with XbaI and PmeI. The obtained DNA fragment except for the DNA sequence encoding the light chain secretory signal and the human κ chain constant region was fused with a DNA fragment comprising a DNA sequence (shown in SEQ ID NO: 20 (FIG. 23) of the Sequence Listing) encoding the amino acids of a human heavy chain signal sequence and a human IgG1 constant region using In-Fusion Advantage PCR cloning kit (Clontech Laboratories, Inc.) to construct a chimeric and humanized IgG1 type heavy chain expression vector pCMA-G1 having the nucleotide sequence encoding a signal sequence, a cloning site, and the human IgG1 heavy chain constant region, downstream of the CMV promoter.

4)-3 Construction of c2A4 Light Chain Expression Vector

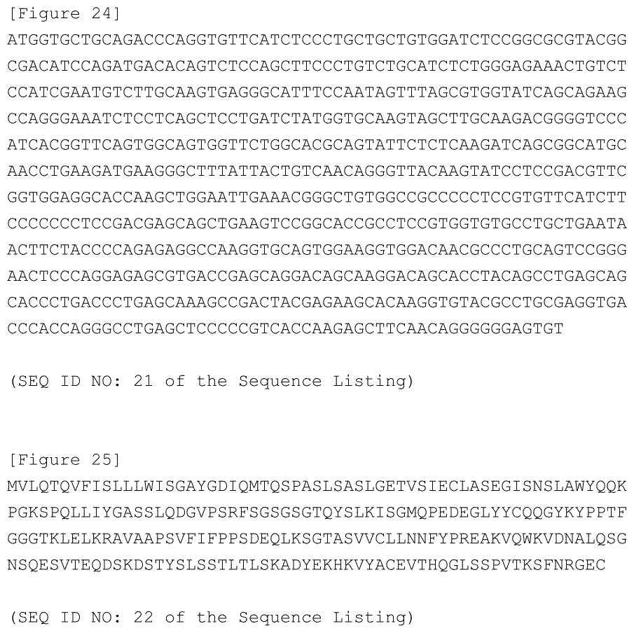

A DNA fragment comprising a light chain variable region-encoding cDNA was amplified by PCR using the 2A4 light chain variable region-encoding cDNA obtained in Example 3) as a template and a primer set shown below, and inserted to the restriction enzyme BsiWI-cleaved site of the chimeric and humanized antibody light chain expression vector pCMA-LK using In-Fusion Advantage PCR cloning kit (Clontech Laboratories, Inc.) to construct a c2A4 light chain expression vector. The obtained expression vector was designated as “pCMA-LK/c2A4”. The nucleotide sequence of the c2A4 light chain and the amino acid sequence of this light chain are shown in SEQ ID NOs: 21 and 22 (FIGS. 24 and 25), respectively, of the Sequence Listing.

Primer Set for c2A4 Light Chain

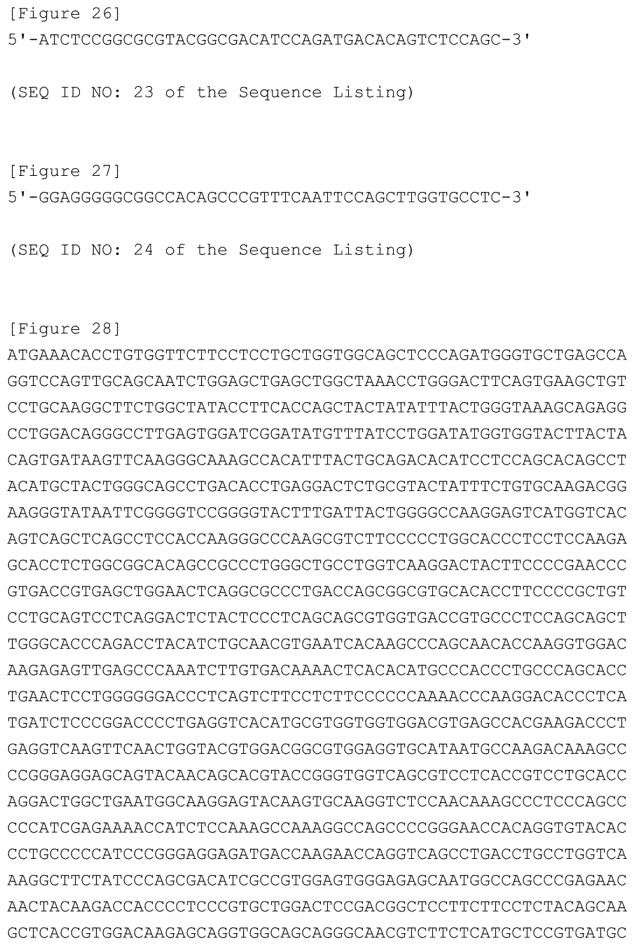

5′-ATCTCCGGCGCGTACGGCGACATCCAGATGACACAGTCTCCAGC-3′
(c2A4-LF: SEQ ID NO: 23 of the Sequence Listing;
FIG. 26)
5′-GGAGGGGGCGGCCACAGCCCGTTTCAATTCCAGCTTGGTGCCTC-3′
(c2A4-LR: SEQ ID NO: 24 of the Sequence Listing;
FIG. 27)

4)-4 Construction of c2A4 Heavy Chain Expression Vector

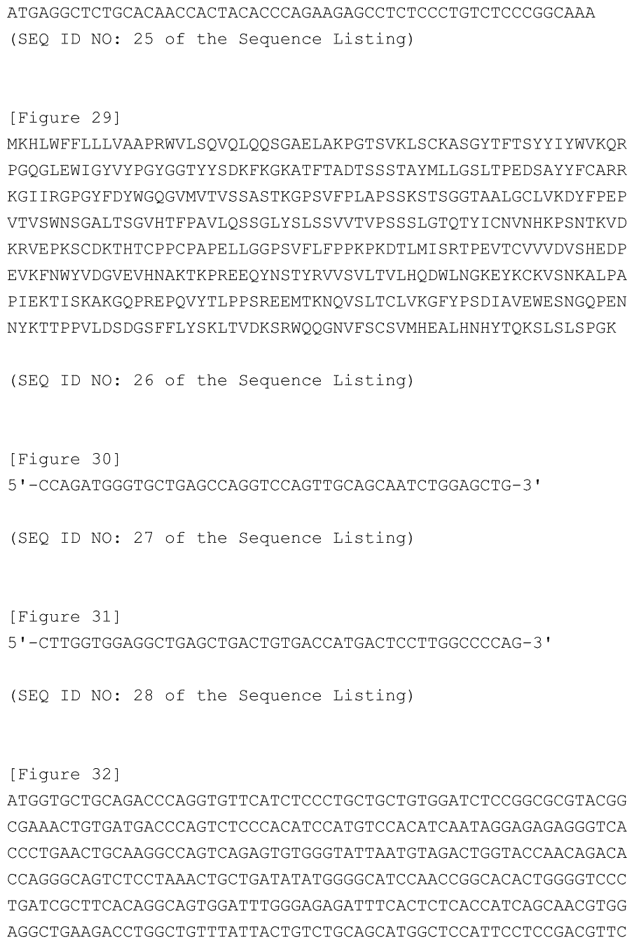

A DNA fragment comprising a heavy chain variable region-encoding cDNA was amplified by PCR using the 2A4 heavy chain variable region-encoding cDNA obtained in Example 3) as a template and a primer set shown below, and inserted to the restriction enzyme B1pI-cleaved site of the chimeric and humanized antibody heavy chain expression vector pCMA-G1 using In-Fusion Advantage PCR cloning kit (Clontech Laboratories, Inc.) to construct a c2A4 heavy chain expression vector. The obtained expression vector was designated as “pCMA-G1/c2A4”. The nucleotide sequence of the c2A4 heavy chain and the amino acid sequence of this heavy chain are shown in SEQ ID NOs: 25 and 26 (FIGS. 28 and 29), respectively, of the Sequence Listing.

Primer Set for c2A4 Heavy Chain

5′-CCAGATGGGTGCTGAGCCAGGTCCAGTTGCAGCAATCTGGAGCTG-
3′ (c2A4-HF: SEQ ID NO: 27 of the Sequence
Listing; FIG. 30)
5′-CTTGGTGGAGGCTGAGCTGACTGTGACCATGACTCCTTGGCCCCAG-
3′ (c2A4-HR: SEQ ID NO: 28 of the Sequence
Listing; FIG. 31

4)-5 Construction of c2B1 Light Chain Expression Vector

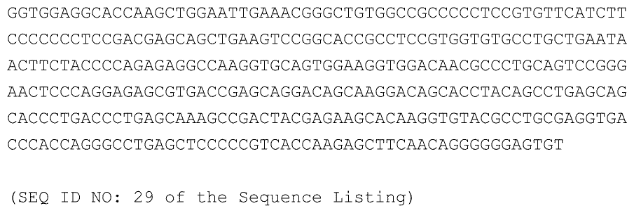

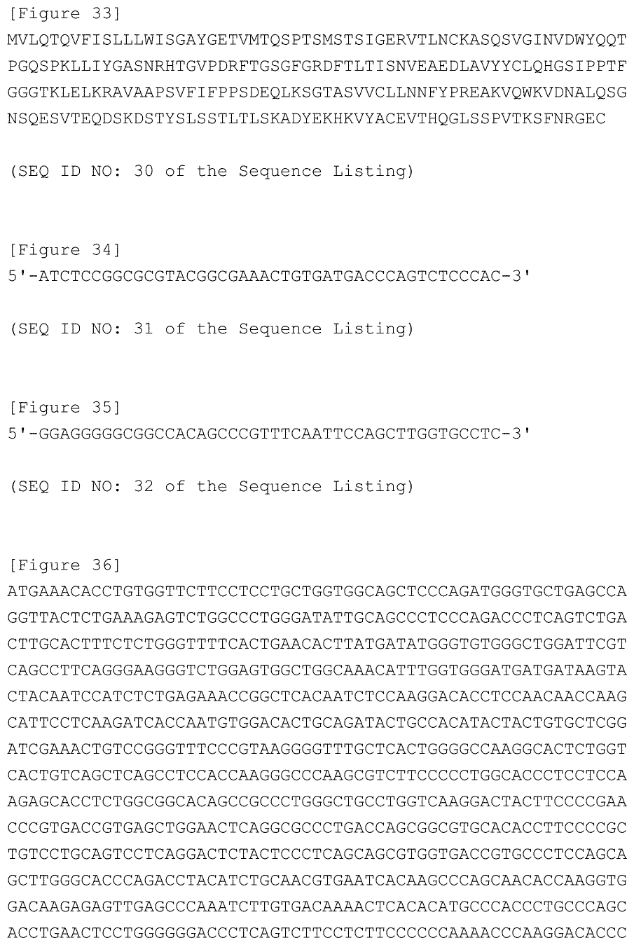

A DNA fragment comprising a light chain variable region-encoding cDNA was amplified by PCR using the 2B1 light chain variable region-encoding cDNA obtained in Example 3) as a template and a primer set shown below. A c2B1 light chain expression vector was constructed in the same way as in Example 4)-3. The obtained expression vector was designated as “pCMA-LK/c2B1”. The nucleotide sequence of the c2B1 light chain and the amino acid sequence of this light chain are shown in SEQ ID NOs: 29 and 30 (FIGS. 32 and 33), respectively, of the Sequence Listing.

Primer Set for c2B1 Light Chain

5′-ATCTCCGGCGCGTACGGCGAAACTGTGATGACCCAGTCTCCCAC-3′
(c2B1-LF: SEQ ID NO: 31 of the Sequence Listing;
FIG. 34)
5′-GGAGGGGGCGGCCACAGCCCGTTTCAATTCCAGCTTGGTGCCTC-3′
(c2B1-LR: SEQ ID NO: 32 of the Sequence Listing;
FIG. 35)

4)-6 Construction of c2B1 Heavy Chain Expression Vector

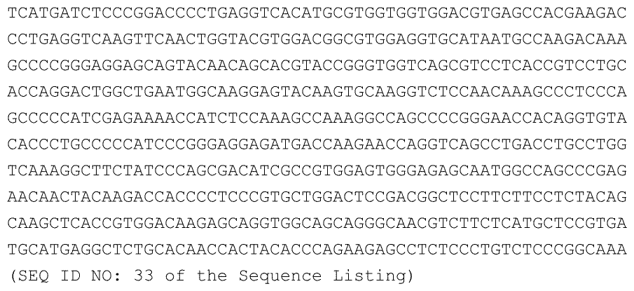

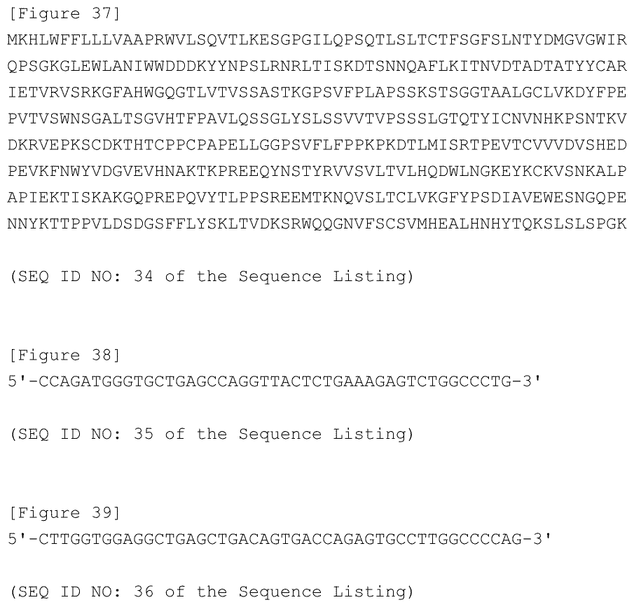

A DNA fragment comprising a heavy chain variable region-encoding cDNA was amplified by PCR using the 2B1 heavy chain variable region-encoding cDNA obtained in Example 3) as a template and a primer set shown below. A c2B1 heavy chain expression vector was constructed in the same way as in Example 4)-4. The obtained expression vector was designated as “pCMA-G1/c2B1”. The nucleotide sequence of the c2B1 heavy chain and the amino acid sequence of this heavy chain are shown in SEQ ID NOs: 33 and 34 (FIGS. 36 and 37), respectively, of the Sequence Listing.

Primer Set for c2B1 Heavy Chain

5′-CCAGATGGGTGCTGAGCCAGGTTACTCTGAAAGAGTCTGGCCCTG-
3′ (c2B1-HF: SEQ ID NO: 35 of the Sequence
Listing; FIG. 38)
5′-CTTGGTGGAGGCTGAGCTGACAGTGACCAGAGTGCCTTGGCCCCAG-
3′ (c2B1-HR: SEQ ID NO: 36 of the Sequence Listing;
FIG. 39)

4)-7 Construction of c7B4 Light Chain Expression Vector

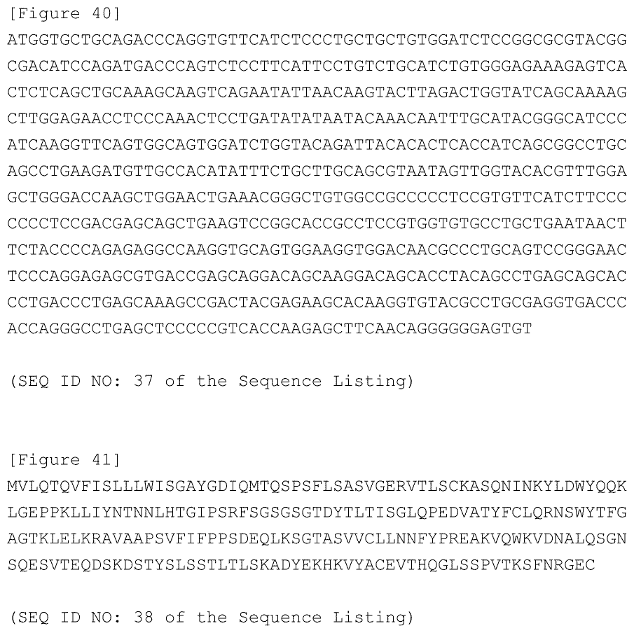

A DNA fragment comprising a light chain variable region-encoding cDNA was amplified by PCR using the 7B4 light chain variable region-encoding cDNA obtained in Example 3) as a template and a primer set shown below. A c7B4 light chain expression vector was constructed in the same way as in Example 4)-3. The obtained expression vector was designated as “pCMA-LK/c7B4”. The nucleotide sequence of the c7B4 light chain and the amino acid sequence of this light chain are shown in SEQ ID NOs: 37 and 38 (FIGS. 40 and 41), respectively, of the Sequence Listing.

Primer Set for c7B4 Light Chain

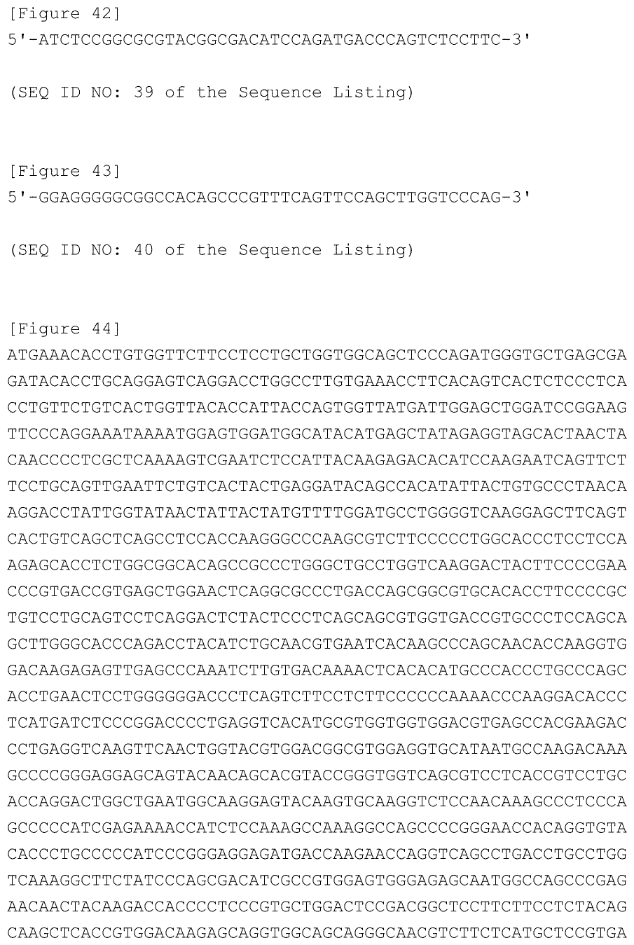

5′-ATCTCCGGCGCGTACGGCGACATCCAGATGACCCAGTCTCCTTC-3′
(c7B4-LF: SEQ ID NO: 39 of the Sequence Listing;
FIG. 42)
5′-GGAGGGGGCGGCCACAGCCCGTTTCAGTTCCAGCTTGGTCCCAG-3′
(c7B4-LR: SEQ ID NO: 40 of the Sequence Listing;
FIG. 43)

4)-8 Construction of c7B4 Heavy Chain Expression Vector

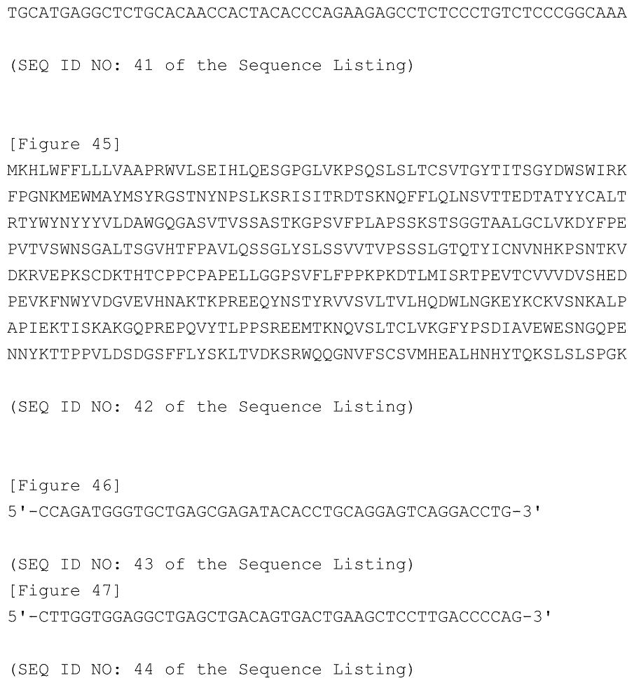

A DNA fragment comprising a heavy chain variable region-encoding cDNA was amplified by PCR using the 7B4 heavy chain variable region-encoding cDNA obtained in Example 3) as a template and a primer set shown below. A c7B4 heavy chain expression vector was constructed in the same way as in Example 4)-4. The obtained expression vector was designated as “pCMA-G1/c7B4”. The nucleotide sequence of the c7B4 heavy chain and the amino acid sequence of this heavy chain are shown in SEQ ID NOs: 41 and 42 (FIGS. 44 and 45), respectively, of the Sequence Listing.

Primer Set for c7B4 Heavy Chain

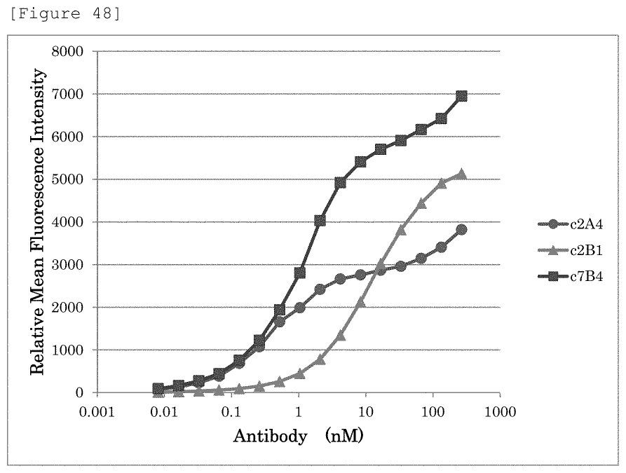

5′-CCAGATGGGTGCTGAGCGAGATACACCTGCAGGAGTCAGGACCTG-
3′ (c7B4-HF: SEQ ID NO: 43 of the Sequence
Listing; FIG. 46)
5′-CTTGGTGGAGGCTGAGCTGACAGTGACTGAAGCTCCTTGACCCCAG-
3′ (c7B4-HR: SEQ ID NO: 44 of the Sequence
Listing; FIG. 47)

### 4)-9 Preparation of Human Chimeric Anti-GPRC5D Antibody

### 4)-9-1 Production of Human Chimeric Anti-GPRC5D Antibody

FreeStyle 293F cells (Invitrogen Corp.) were subcultured and cultured according to the manual. 1.2×10⁹ FreeStyle 293F cells (Invitrogen Corp.) in the logarithmic growth phase were inoculated to a 3-L Fernbach Erlenmeyer Flask (Corning Inc.), adjusted to 2.0×10⁶ cells/ml by dilution with FreeStyle 293 expression medium (Invitrogen Corp.), and then shake-cultured at 90 rpm at 37° C. for 1 hour in an 8% CO₂ incubator. 1.8 mg of polyethyleneimine (Polysciences #24765) was dissolved in 20 ml of Opti-Pro SFM medium (Invitrogen Corp.). Next, each heavy chain expression vector (0.24 mg) and light chain expression vector (0.36 mg) prepared using NucleoBond Xtra (Takara Bio Inc.) were added to 20 ml of Opti-Pro SFM medium (Invitrogen Corp.). 20 ml of the expression vector/Opti-Pro SFM mixed solution was added to 20 ml of the polyethyleneimine/Opti-Pro SFM mixed solution, and the mixture was gently stirred, left for 5 minutes, and then added to the FreeStyle 293F cells. The cells were shake-cultured at 90 rpm at 37° C. for 4 hours in an 8% CO₂ incubator. Then, 600 ml of EX-CELL VPRO medium (SAFC Biosciences Inc.), 18 ml of GlutaMAX I (Gibco/Thermo Fisher Scientific Inc.), and 30 ml of Yeastolate Ultrafiltrate (Gibco/Thermo Fisher Scientific Inc.) were added thereto. The cells were shake-cultured at 90 rpm at 37° C. for 7 days in an 8% CO₂ incubator, and the obtained culture supernatant was filtered through Disposable Capsule Filter (Advantec #CCS-045-E1H).

The human chimeric 2A4 obtained by the combination of pCMA-G1/c2A4 and pCMA-LK/c2A4 was designated as “c2A4”. The human chimeric 2B1 obtained by the combination of pCMA-G1/c2B1 and pCMA-LK/c2B1 was designated as “c2B1”. The human chimeric 7B4 obtained by the combination of pCMA-G1/c7B4 and pCMA-LK/c7B4 was designated as “c7B4”.

### 4)-9-2 Purification of Human Chimeric Anti-GPRC5D Antibody

Each antibody was purified from the culture supernatant obtained in Example 4)-9-1 in one step by rProtein A affinity chromatography (at 4 to 6° C.) A buffer replacement step after the rProtein A affinity chromatography purification was carried out at 4 to 6° C. The culture supernatant was applied to a column packed with MabSelectSuRe (manufactured by GE Healthcare Bio-Sciences Corp.) equilibrated with PBS. After entry of the whole culture solution into the column, the column was washed with PBS in an amount of twice or more the column volume. Next, antibody-containing fractions were collected by elution with a 2 M arginine hydrochloride solution (pH 4.0). The collected fractions were buffer-replaced with HBSor (25 mM histidine/5% sorbitol, pH 6.0) by dialysis (Thermo Fisher Scientific Inc., Slide-A-Lyzer Dialysis Cassette). The antibody was concentrated and adjusted to an IgG concentration of 10 mg/ml or higher using Centrifugal UF Filter Device VIVASPIN20 (molecular weight cutoff: UF10K, Sartorius Japan K.K., 4° C.), and used as a purified sample. Finally, this purified sample was filtered through Minisart-Plus filter (Sartorius Japan K.K.).

### Example 5

### In Vitro Activity of Human Chimeric Anti-GPRC5D Antibodies (c2A4, c2B1, and c7B4)

5)-1 Study on Binding Activity of Human Chimeric Anti-GPRC5D Antibodies (c2A4, c2B1, and c7B4) Against Human GPRC5D by Flow Cytometry

Human multiple myeloma cell line KHM-1B cells expressing GPRC5D were adjusted to a concentration of 5×10⁶ cells/mL with PBS containing 5% FBS, inoculated in an amount of 100 μL/well to a 96-well U-bottomed microplate, and centrifuged to remove a supernatant. Each human chimeric anti-GPRC5D antibody (c2A4, c2B1, and c7B4) or Human IgG isotype control antibody (Calbiochem/Merck Millipore Corp.) adjusted to 1.2 ng/mL to 40 μg/mL was added thereto in an amount of 100 μL/well, and the plate was left standing at 4° C. for 1 hour. The cells were washed twice with PBS containing 5% FBS. Then, R-Phycoerythrin AffiniPure F(ab′)2 Fragment Goat Anti-Human IgG, Fcγ Fragment Specific (Jackson ImmunoResearch Laboratories, Inc.) diluted 100-fold with PBS containing 5% FBS was added thereto in an amount of 100 μL/well, and the plate was left standing at 4° C. for 1 hour. The cells were washed twice with PBS containing 5% FBS and then resuspended in PBS containing 5% FBS, followed by detection using a flow cytometer (FACSCanto™ II; Becton, Dickinson and Company). The data was analyzed using Flowjo (Tree Star, Inc.). The PE fluorescence intensity of the cell fraction was plotted to a histogram, and the mean fluorescence intensity (MFI) was calculated. The MFI value of the control antibody was subtracted from the MFI value of the GPRC5D antibody to calculate a relative value of MFI (I). As shown in FIG. 48, c2A4, c2B1, and c7B4 were found to bind to human GPRC5D.

5)-2 Study on Cross-Reactivity of Human Chimeric Anti-GPRC5D Antibodies (c2A4, c2B1, and c7B4) with Cynomolgus Monkey GPRC5D
5)-2-1 Construction of Cynomolgus Monkey GPRC5D Expression Vector (pcDNA3.1-DEST-cGPRC5D)

A cDNA encoding the cynomolgus monkey GPRC5D protein (XP_005570249.1) was cloned into the pcDNA3.1-DEST vector prepared in Example 1)-1-1 using Gateway LR Clonase Enzyme mix (Life Technologies Corp.) to construct a cynomolgus monkey GPRC5D expression vector pcDNA3.1-DEST-cGPRC5D. For the large-scale preparation of the cynomolgus monkey GPRC5D expression vector, Endofree Plasmid Giga Kit (Qiagen N.V.) was used.

### 5)-2-2 Preparation of Cynomolgus Monkey GPRC5D-Expressing Cell Line

A multiple myeloma cell line KMS-11 (JCRB Cell Bank) expressing no human GPRC5D was inoculated at a concentration of 3.7×10³ cells/mL to a 75-cm² flask using an RPMI1640 medium containing 10% FBS. pcDNA3.1-DEST-cGPRC5D was transfected to the KMS-11 cells using Lipofectamine 2000 (Thermo Fisher Scientific Inc.), and the cells were cultured at 37° C. for 2 days under 5% CO₂ conditions. The cultured expression vector-transfected KMS-11_cells were cultured at a concentration of 1×10⁶ cells/mL in an RPMI1640 medium containing 10% FBS and 1 mg/mL Geneticin (Thermo Fisher Scientific Inc.) for drug screening. Bulk cells were prepared as single clones by the limiting dilution method using ClonaCell-HY Selection Medium E medium (StemCell Technologies Inc.) containing 1 mg/mL Geneticin (Thermo Fisher Scientific Inc.) to establish a cynomolgus monkey GPRC5D-expressing multiple myeloma cell line KMS-11_cGPRC5D.

5)-2-3 Study on Binding Activity of Human Chimeric Anti-GPRC5D Antibodies (c2A4, c2B1, and c7B4) Against Cynomolgus Monkey GPRC5D by Flow Cytometry

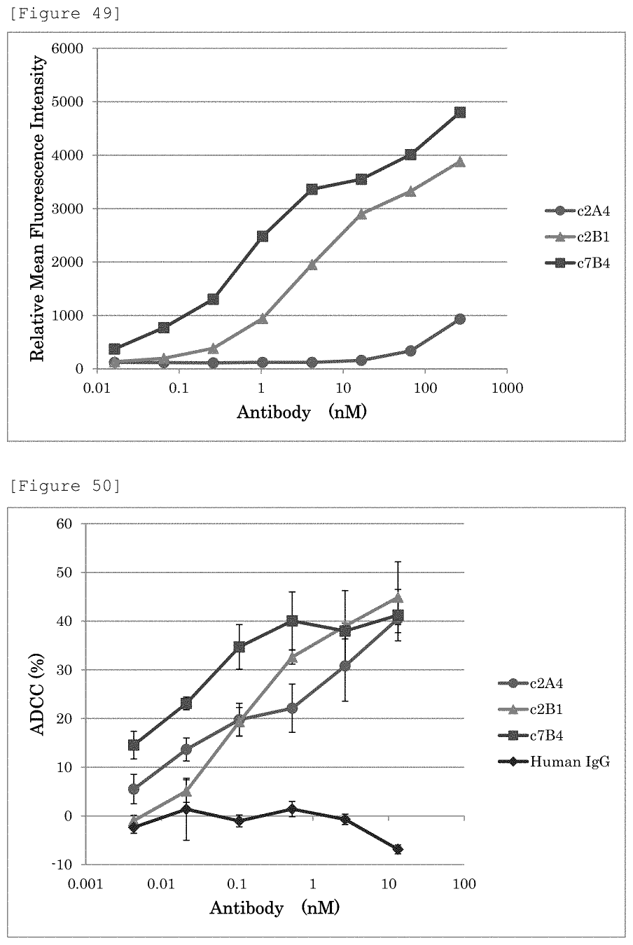

The KMS-11_cGPRC5D cells prepared in Example 5)-2-2 were adjusted to a concentration of 5×10⁶ cells/mL with PBS containing 5% FBS, inoculated in an amount of 100 μL/well to a 96-well U-bottomed microplate, and centrifuged to remove a supernatant. Each human chimeric anti-GPRC5D antibody (c2A4, c2B1, and c7B4) or Human IgG isotype control antibody (Calbiochem/Merck Millipore Corp.) adjusted to 1.2 ng/mL to 40 μg/mL was added thereto in an amount of 100 μL/well, and the plate was left standing at 4° C. for 1 hour. The cells were washed twice with PBS containing 5% FBS. Then, R-Phycoerythrin AffiniPure F(ab′)2 Fragment Goat Anti-Human IgG, Fcγ Fragment Specific (Jackson ImmunoResearch Laboratories, Inc.) diluted 100-fold with PBS containing 5% FBS was added thereto in an amount of 100 μL/well, and the plate was left standing at 4° C. for 1 hour. The cells were washed twice with PBS containing 5% FBS and then resuspended in PBS containing 5% FBS, followed by detection using a flow cytometer (FACSCanto™ II; Becton, Dickinson and Company). The data was analyzed using Flowjo (Tree Star, Inc.). The PE fluorescence intensity of the cell fraction was plotted to a histogram, and the mean fluorescence intensity (MFI) was calculated. The MFI value of the control antibody was subtracted from the MFI value of the GPRC5D antibody to calculate a relative value of MFI (rMFI). As shown in FIG. 49, c2A4, c2B1, and c7B4 were found to bind to cynomolgus monkey GPRC5D. The antibody, etc. binding to human GPRC5D and cynomolgus monkey GPRC5D can be subjected to various tests on efficacy or safety using primates, particularly, cynomolgus monkeys, useful for the nonclinical development (preclinical development) of pharmaceutical products, and are thus preferred. Also, the antibody, etc. binding to human GPRC5D and cynomolgus monkey GPRC5D have cytotoxic activity and are useful, either alone or as the molecule of the present invention, in the treatment or prevention of diseases such as cancers in cynomolgus monkeys.

As a result of studying the cross-reactivity of c2A4, c2B1, and c7B4 with rat GPRC5D and mouse GPRC5D in the same way as in Example 5)-2, none of c2A4, c2B1, and c7B4 bound to rat GPRC5D and mouse GPRC5D. By virtue of these antibodies c2A4, c2B1, and c7B4, various assays, immunohistochemical tests, etc. using human GPRC5D gene-transfected mouse or rat cells, tissues, or individuals (including transgenic animals, knockout animals, and knock-in animals) and the antibody, etc. can be carried out without being influenced by GPRC5D of the host mice. Thus, these antibodies are preferred for the research and nonclinical development, using mice or rats, of drugs, animal drugs, or diagnostic drugs, etc., comprising the antibody, etc.

5)-3 ADCC Activity of Human Chimeric Anti-GPRC5D Antibodies (c2A4, c2B1, and c7B4)

The KHM-1B cells prepared in Example 2)-3-1 were added in an amount of 50 μL/well to a 96-well U-bottomed microplate. Each purified human chimeric anti-GPRC5D antibody (c2A4, c2B1, and c7B4) prepared in Example 4 or a human control antibody (hIgG1) (Calbiochem/Merck Millipore Corp.) adjusted to 0.64 ng/mL to 2 μg/mL (final concentration) was added thereto in an amount of 50 μL/well, and the plate was left standing at 4° C. for 30 minutes. The effector cells (adjusted to 3×10⁶ cells/mL) prepared in Example 1)-5-3 were further added thereto at a concentration of 100 μL/well. After centrifugation at room temperature at 1200 rpm for 3 minutes, the cells were cultured at 37° C. for 4 hours under 5% CO₂ conditions. A 50 μL aliquot of the supernatant was recovered into LumaPlate (PerkinElmer, Inc.) and dried overnight at 50° C., followed by measurement using a plate reader (TopCount; PerkinElmer, Inc.). The percentage of cells lysed by ADCC activity was calculated according to Example 1)-5-5. As shown in FIG. 50, c2A4, c2B1, and c7B4 were found to have ADCC activity.

### Example 6

### In Vivo Activity of Human Chimeric Anti-GPRC5D Antibodies (c2A4, c2B1, and c7B4)

1×10⁷ cells of a human multiple myeloma cell line KHM-1B were suspended in 100% Matrigel (Becton, Dickinson and Company) and subcutaneously transplanted to the axillary region of each BALB/c-nu/nu mouse (CanN.Cg-Foxn1lnu/CrlCrlj, purchased from Charles River Laboratories Japan Inc.). Three and days after transplantation, each human chimeric anti-GPRC5D antibody (c2A4, c2B1, and c7B4) was administered at a dose of 10 mg/kg to the tail veins of the cancer-bearing mice (n=12 or 11). The major axis and minor axis of the transplanted tumor were measured twice a week using an electronic digital caliper (manufactured by Mitsutoyo Corp.). The tumor volume was calculated according to the following expression:

Tumor volume (mm³)=1/2×Minor axis (mm)×Minor axis (mm)×Major axis (mm)

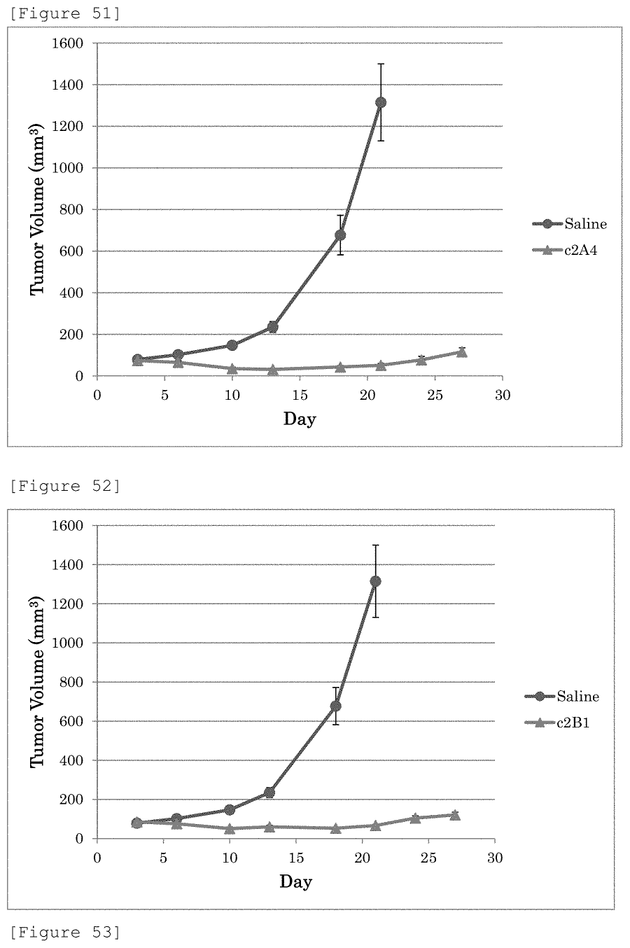

The results on the c2A4 antibody are shown in FIG. 51. The percentage of tumor growth inhibition at 21 days after transplantation was 96%.

The results on the c2B1 antibody are shown in FIG. 52. The percentage of tumor growth inhibition at 21 days after transplantation was 95%.

The results on the c7B4 antibody are shown in FIG. 53. The percentage of tumor growth inhibition at 21 days after transplantation was 94%.

### Example 7

### Design of Humanized Versions (h2B1 and h7B4) of Human Chimeric Anti-GPRC5D Antibodies (c2B1 and c7B4)

### 7)-1 Design of Humanized Form of Anti-GPRC5D Antibody 2B1

### 7)-1-1 Molecular Modeling of 2B1 Variable Regions

The molecular modeling of the 2B1 variable regions was carried out by a method generally known as homology modeling (Methods in Enzymology, 203, 121-153, (1991)). The variable regions of 2B1 determined above were compared with the primary sequences (three-dimensional structures derived from X-ray crystal structures are available) of human immunoglobulin variable regions registered in Protein Data Bank (Nuc. Acid Res. 35, D301-D303 (2007)). As a result, 3MBX was selected because it had the highest sequence identity to the heavy and light chain variable regions of 2B1. The three-dimensional structures of framework regions were prepared as a “framework model” by combining the coordinates of 3MBX corresponding to the heavy and light chains of 2B1. Subsequently, the typical conformation of each CDR was incorporated into the framework model. Finally, energy calculation for excluding disadvantageous interatomic contact was conducted in order to obtain possible molecular models of the 2B1 variable regions in terms of energy. These procedures were performed using a commercially available protein three-dimensional structure analysis program BioLuminate (manufactured by Schrodinger, LLC).

7)-1-2 Design of Amino Acid Sequence of Humanized h2B1

Humanized h2B1 was constructed by a method generally known as CDR grafting (Proc. Natl. Acad. Sci. USA 86, 10029-10033 (1989)). An acceptor antibody was selected on the basis of the identity of amino acids in framework regions.

The sequences of the framework regions of 2B1 were compared with the framework regions of human subgroup consensus sequences or germline sequences specified by KABAT et al. (Sequences of Proteins of Immunological Interest, 5th Ed. Public Health Service National Institutes of Health, Bethesda, Md. (1991)). As a result, human germline sequences IGHV2_5x08 and IGHJ1x01 and a human gamma chain subgroup 2 consensus sequence were selected as a heavy chain acceptor while human germline sequences IGKV1_8x01 and IGKJ4x01 and a human kappa chain subgroup 4 consensus sequence were selected as light chain acceptor due to their high sequence identity as to framework regions. The amino acid residues of the framework regions of the acceptors were aligned with the amino acid residues of the 2B1 framework regions to identify the positions of amino acids that did not match therebetween. The positions of these residues were analyzed using the three-dimensional model of 2B1 constructed above. Then, the donor residues to be grafted onto the acceptors were selected according to the criteria provided by Queen et al. (Proc. Natl. Acad. Sci. USA 86, 10029-10033 (1989)). Some donor residues thus selected were transferred to the acceptor antibody to construct the humanized h2B1 sequence as described in Examples below. The heavy chain was not limited by donor residues, and depending on a site, residues of a gamma chain subgroup 1 consensus sequence were transferred thereto.

### 7)-1-3 Humanization of 2B1 Heavy Chain

7)-1-3-1 Humanized h2B1_H1 Type Heavy Chain

A humanized h2B1 heavy chain designed from the chimeric c2B1 heavy chain shown in SEQ ID NO: 34 by the replacement in the variable region of threonine at amino acid position 3 with glutamine, lysine at amino acid position 5 with valine, proline at amino acid position 9 with glycine, isoleucine at amino acid position 11 with leucine, leucine at amino acid position 12 with valine, glutamine at amino acid position 13 with lysine, serine at amino acid position 43 with proline, leucine at amino acid position 50 with isoleucine, alanine at amino acid position 51 with glycine, arginine at amino acid position 66 with lysine, asparagine at amino acid position 67 with serine, leucine at amino acid position 69 with valine, lysine at amino acid position 73 with valine, asparagine at amino acid position 77 with lysine, phenylalanine at amino acid position 81 with serine, isoleucine at amino acid position 84 with leucine, threonine at amino acid position 85 with serine, asparagine at amino acid position 86 with serine, aspartic acid at amino acid position 88 with threonine, threonine at amino acid position 89 with alanine, and threonine at amino acid position 94 with valine was designated as “humanized h2B1_H1 type heavy chain” (also referred to as “h2B1_H1”).

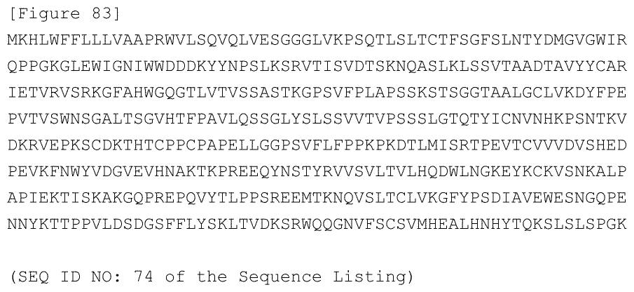

The amino acid sequence of the humanized h2B1_H1 type heavy chain is described in SEQ ID NO: 74 (FIG. 83) of the Sequence Listing. A nucleotide sequence encoding the amino acid sequence of SEQ ID NO: 74 is described in SEQ ID NO: 73 (FIG. 82) of the Sequence Listing.

7)-1-3-2 Humanized h2B1_H2 Type Heavy Chain

A humanized h2B1 heavy chain designed from the chimeric c2B1 heavy chain shown in SEQ ID NO: 34 by the replacement in the variable region of threonine at amino acid position 3 with glutamine, lysine at amino acid position 5 with valine, proline at amino acid position 9 with glycine, isoleucine at amino acid position 11 with leucine, leucine at amino acid position 12 with valine, glutamine at amino acid position 13 with lysine, serine at amino acid position 43 with proline, leucine at amino acid position 50 with isoleucine, alanine at amino acid position 51 with glycine, arginine at amino acid position 66 with lysine, asparagine at amino acid position 67 with serine, leucine at amino acid position 69 with valine, phenylalanine at amino acid position 81 with serine, isoleucine at amino acid position 84 with leucine, threonine at amino acid position 85 with serine, asparagine at amino acid position 86 with serine, aspartic acid at amino acid position 88 with threonine, threonine at amino acid position 89 with alanine, and threonine at amino acid position 94 with valine was designated as “humanized h2B1_H2 type heavy chain” (also referred to as “h2B1_H2”).

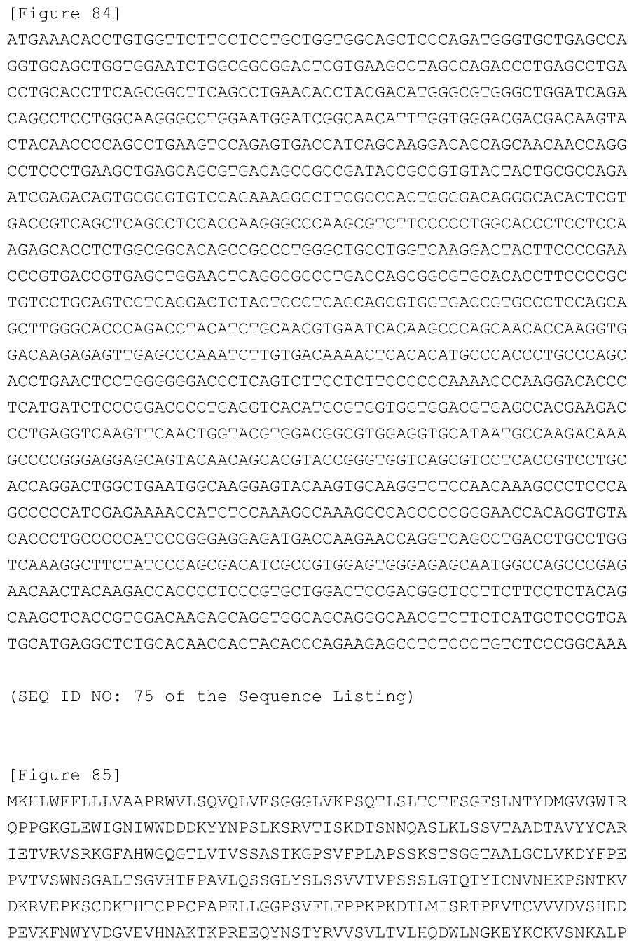

The amino acid sequence of the humanized h2B1_H2 type heavy chain is described in SEQ ID NO: 76 (FIG. 85) of the Sequence Listing. A nucleotide sequence encoding the amino acid sequence of SEQ ID NO: 76 is described in SEQ ID NO: 75 (FIG. 84) of the Sequence Listing.

7)-1-3-3 Humanized h2B1_H3 Type Heavy Chain

A humanized h2B1 heavy chain designed from the chimeric c2B1 heavy chain shown in SEQ ID NO: 34 by the replacement in the variable region of threonine at amino acid position 3 with glutamine, lysine at amino acid position 5 with valine, proline at amino acid position 9 with glycine, isoleucine at amino acid position 11 with leucine, leucine at amino acid position 12 with valine, glutamine at amino acid position 13 with lysine, serine at amino acid position 43 with proline, leucine at amino acid position 50 with isoleucine, arginine at amino acid position 66 with lysine, asparagine at amino acid position 67 with serine, leucine at amino acid position 69 with valine, phenylalanine at amino acid position 81 with serine, isoleucine at amino acid position 84 with leucine, threonine at amino acid position 85 with serine, asparagine at amino acid position 86 with serine, aspartic acid at amino acid position 88 with threonine, threonine at amino acid position 89 with alanine, and threonine at amino acid position 94 with valine was designated as “humanized h2B1_H3 type heavy chain” (also referred to as “h2B1_H3”).

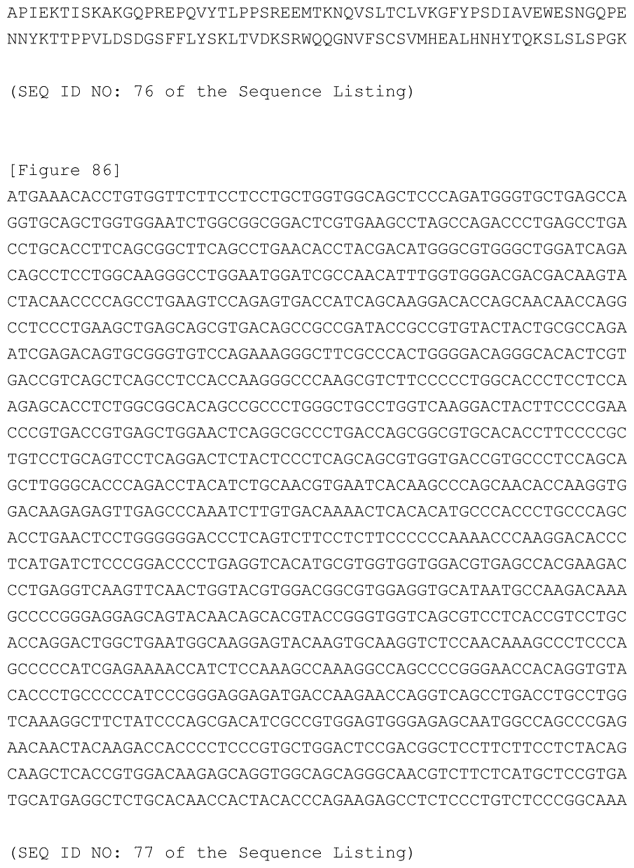

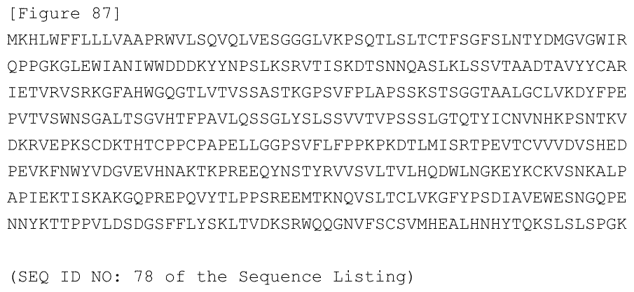

The amino acid sequence of the humanized h2B1_H3 type heavy chain is described in SEQ ID NO: 78 (FIG. 87) of the Sequence Listing. A nucleotide sequence encoding the amino acid sequence of SEQ ID NO: 78 is described in SEQ ID NO: 77 (FIG. 86) of the Sequence Listing.

7)-1-3-4 Humanized h2B1_H4 Type Heavy Chain

A humanized h2B1 heavy chain designed from the chimeric c2B1 heavy chain shown in SEQ ID NO: 34 by the replacement in the variable region of glycine at amino acid position 10 with alanine, isoleucine at amino acid position 11 with leucine, leucine at amino acid position 12 with valine, glutamine at amino acid position 13 with lysine, serine at amino acid position 15 with threonine, serine at amino acid position 19 with threonine, serine at amino acid position 43 with proline, asparagine at amino acid position 62 with serine, arginine at amino acid position 66 with lysine, asparagine at amino acid position 67 with serine, serine at amino acid position 72 with threonine, phenylalanine at amino acid position 81 with valine, lysine at amino acid position 83 with threonine, isoleucine at amino acid position 84 with methionine, valine at amino acid position 87 with methionine, threonine at amino acid position 89 with proline, and alanine at amino acid position 90 with valine was designated as “humanized h2B1_H4 type heavy chain” (also referred to as “h2B1_H4”).

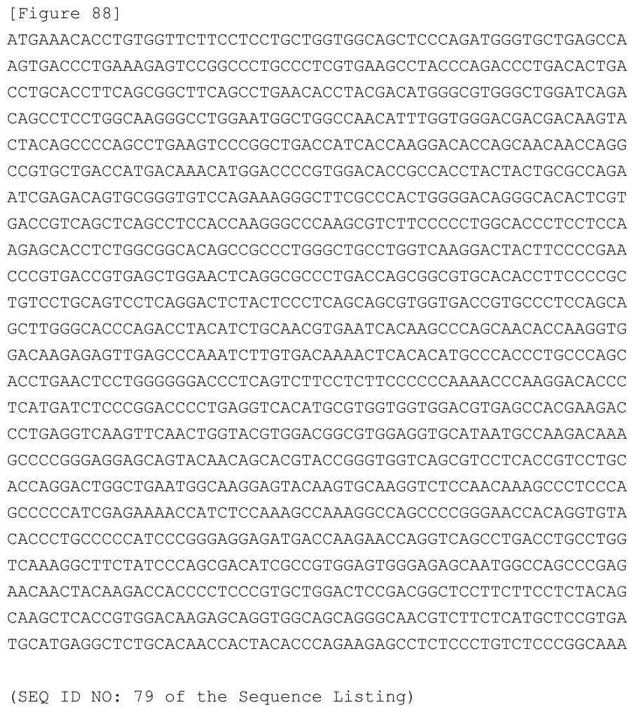

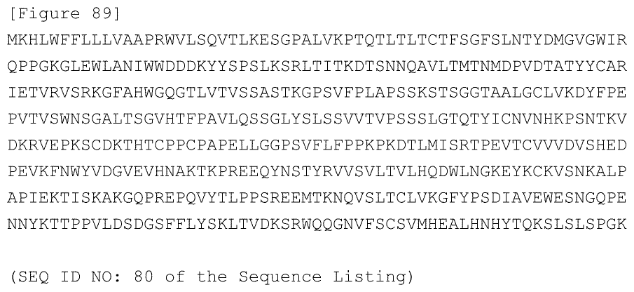

The amino acid sequence of the humanized h2B1_H4 type heavy chain is described in SEQ ID NO: 80 (FIG. 89) of the Sequence Listing. A nucleotide sequence encoding the amino acid sequence of SEQ ID NO: 80 is described in SEQ ID NO: 79 (FIG. 88) of the Sequence Listing.

### 7)-1-4 Humanization of 2B1 Light Chain

7)-1-4-1 Humanized h2B1_L1 Type Light Chain

A humanized h2B1 light chain designed from the chimeric c2B1 light chain shown in SEQ ID NO: 30 by the replacement in the variable region of glutamic acid at amino acid position 1 with aspartic acid, threonine at amino acid position 9 with aspartic acid, methionine at amino acid position 11 with leucine, serine at amino acid position 12 with alanine, threonine at amino acid position 13 with valine, isoleucine at amino acid position 15 with leucine, valine at amino acid position 19 with alanine, leucine at amino acid position 21 with isoleucine, threonine at amino acid position 39 with lysine, serine at amino acid position 43 with proline, threonine at amino acid position 63 with serine, phenylalanine at amino acid position 67 with serine, asparagine at amino acid position 77 with serine, valine at amino acid position 78 with leucine, glutamic acid at amino acid position 79 with glutamine, leucine at amino acid position 83 with valine, glycine at amino acid position 100 with glutamine, leucine at amino acid position 104 with valine, and leucine at amino acid position 106 with isoleucine was designated as “humanized h2B1_L1 type light chain” (also referred to as “h2B1_L1”).

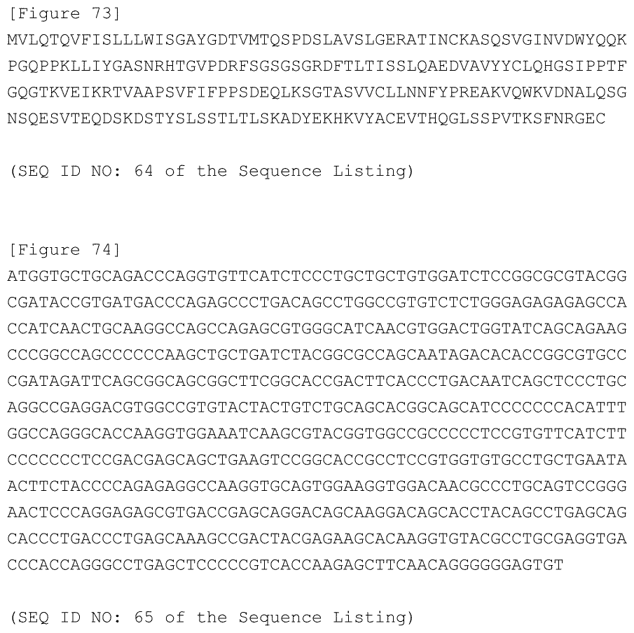

The amino acid sequence of the humanized h2B1_L1 type light chain is described in SEQ ID NO: 64 (FIG. 73) of the Sequence Listing. A nucleotide sequence encoding the amino acid sequence of SEQ ID NO: 64 is described in SEQ ID NO: 63 (FIG. 72) of the Sequence Listing.

7)-1-4-2 Humanized h2B1_L2 Type Light Chain

A humanized h2B1 light chain designed from the chimeric c2B1 light chain shown in SEQ ID NO: 30 by the replacement in the variable region of glutamic acid at amino acid position 1 with aspartic acid, threonine at amino acid position 9 with aspartic acid, methionine at amino acid position 11 with leucine, serine at amino acid position 12 with alanine, threonine at amino acid position 13 with valine, isoleucine at amino acid position 15 with leucine, valine at amino acid position 19 with alanine, leucine at amino acid position 21 with isoleucine, threonine at amino acid position 39 with lysine, serine at amino acid position 43 with proline, threonine at amino acid position 63 with serine, arginine at amino acid position 69 with threonine, asparagine at amino acid position 77 with serine, valine at amino acid position 78 with leucine, glutamic acid at amino acid position 79 with glutamine, leucine at amino acid position 83 with valine, glycine at amino acid position 100 with glutamine, leucine at amino acid position 104 with valine, and leucine at amino acid position 106 with isoleucine was designated as “humanized h2B1_L2 type light chain” (also referred to as “h2B1_L2”).

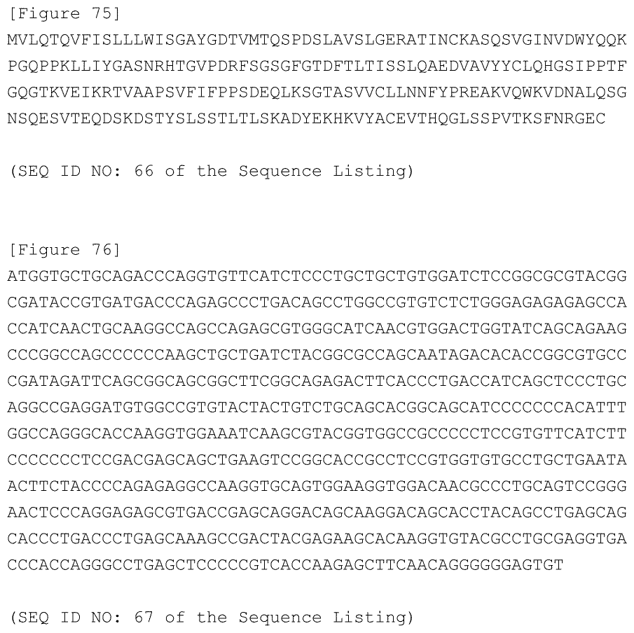

The amino acid sequence of the humanized h2B1_L2 type light chain is described in SEQ ID NO: 66 (FIG. 75) of the Sequence Listing. A nucleotide sequence encoding the amino acid sequence of SEQ ID NO: 66 is described in SEQ ID NO: 65 (FIG. 74) of the Sequence Listing.

7)-1-4-3 Humanized h2B1_L3 Type Light Chain

A humanized h2B1 light chain designed from the chimeric c2B1 light chain shown in SEQ ID NO: 30 by the replacement in the variable region of glutamic acid at amino acid position 1 with aspartic acid, threonine at amino acid position 9 with aspartic acid, methionine at amino acid position 11 with leucine, serine at amino acid position 12 with alanine, threonine at amino acid position 13 with valine, isoleucine at amino acid position 15 with leucine, valine at amino acid position 19 with alanine, leucine at amino acid position 21 with isoleucine, threonine at amino acid position 39 with lysine, serine at amino acid position 43 with proline, threonine at amino acid position 63 with serine, asparagine at amino acid position 77 with serine, valine at amino acid position 78 with leucine, glutamic acid at amino acid position 79 with glutamine, leucine at amino acid position 83 with valine, glycine at amino acid position 100 with glutamine, leucine at amino acid position 104 with valine, and leucine at amino acid position 106 with isoleucine was designated as “humanized h2B1_L3 type light chain” (also referred to as “h2B1_L3”).

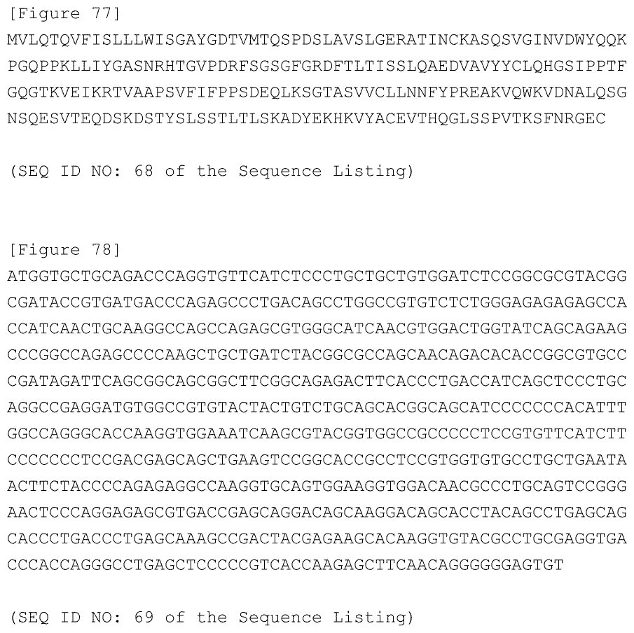

The amino acid sequence of the humanized h2B1_L3 type light chain is described in SEQ ID NO: 68 (FIG. 77) of the Sequence Listing. A nucleotide sequence encoding the amino acid sequence of SEQ ID NO: 68 is described in SEQ ID NO: 67 (FIG. 76) of the Sequence Listing.

7)-1-4-4 Humanized h2B1_L4 Type Light Chain

A humanized h2B1 light chain designed from the chimeric c2B1 light chain shown in SEQ ID NO: 30 by the replacement in the variable region of glutamic acid at amino acid position 1 with aspartic acid, threonine at amino acid position 9 with aspartic acid, methionine at amino acid position 11 with leucine, serine at amino acid position 12 with alanine, threonine at amino acid position 13 with valine, isoleucine at amino acid position 15 with leucine, valine at amino acid position 19 with alanine, leucine at amino acid position 21 with isoleucine, threonine at amino acid position 39 with lysine, threonine at amino acid position 63 with serine, asparagine at amino acid position 77 with serine, valine at amino acid position 78 with leucine, glutamic acid at amino acid position 79 with glutamine, leucine at amino acid position 83 with valine, glycine at amino acid position 100 with glutamine, leucine at amino acid position 104 with valine, and leucine at amino acid position 106 with isoleucine was designated as “humanized h2B1_L4 type light chain” (also referred to as “h2B1_L4”).

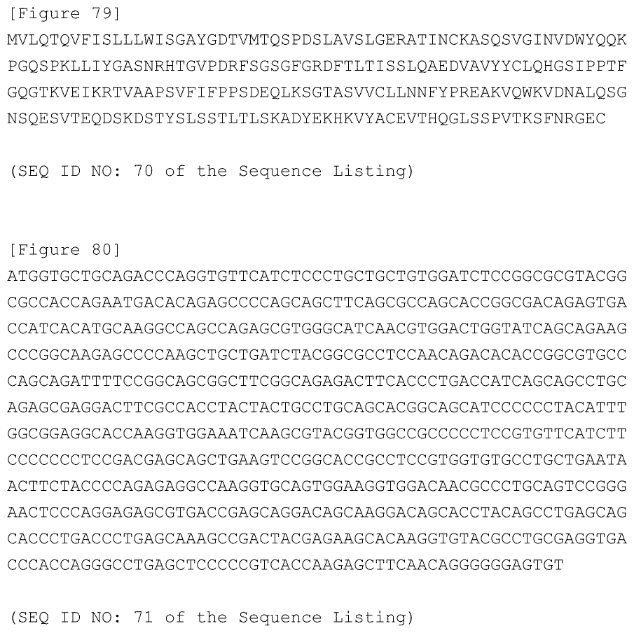

The amino acid sequence of the humanized h2B1_L4 type light chain is described in SEQ ID NO: 70 (FIG. 79) of the Sequence Listing. A nucleotide sequence encoding the amino acid sequence of SEQ ID NO: 70 is described in SEQ ID NO: 69 (FIG. 78) of the Sequence Listing.

7)-1-4-5 Humanized h2B1_L5 Type Light Chain

A humanized h2B1 light chain designed from the chimeric c2B1 light chain shown in SEQ ID NO: 30 by the replacement in the variable region of glutamic acid at amino acid position 1 with alanine, valine at amino acid position 3 with arginine, threonine at amino acid position 9 with serine, methionine at amino acid position 11 with phenylalanine, threonine at amino acid position 13 with alanine, isoleucine at amino acid position 15 with threonine, glutamic acid at amino acid position 17 with aspartic acid, leucine at amino acid position 21 with isoleucine, asparagine at amino acid position 22 with threonine, threonine at amino acid position 39 with lysine, glutamine at amino acid position 42 with lysine, aspartic acid at amino acid position 60 with serine, threonine at amino acid position 63 with serine, asparagine at amino acid position 77 with serine, valine at amino acid position 78 with leucine, glutamic acid at amino acid position 79 with glutamine, alanine at amino acid position 80 with serine, leucine at amino acid position 83 with phenylalanine, valine at amino acid position 85 with threonine, leucine at amino acid position 104 with valine, and leucine at amino acid position 106 with isoleucine was designated as “humanized h2B1_L5 type light chain” (also referred to as “h2B1_L5”).

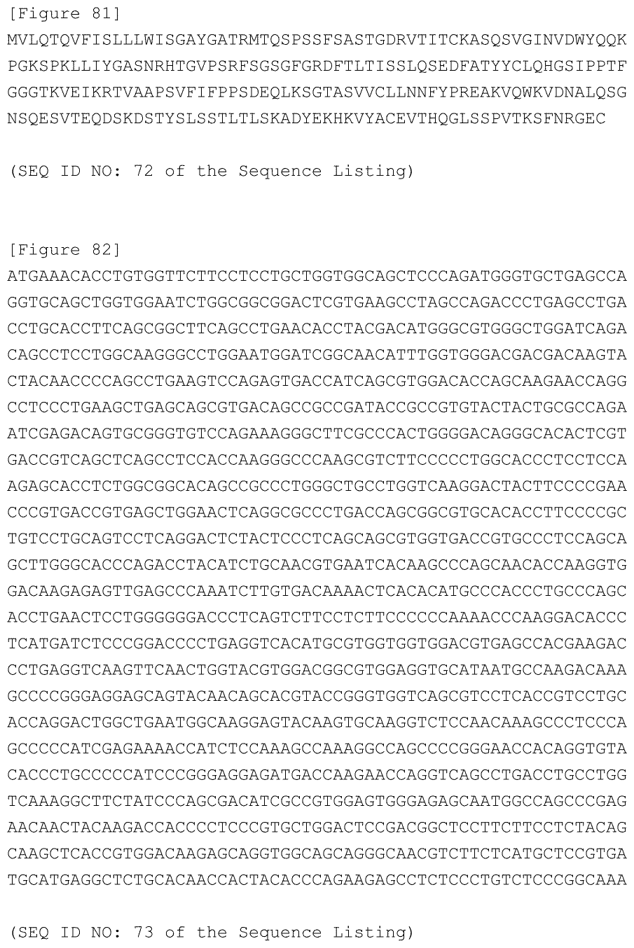

The amino acid sequence of the humanized h2B1_L5 type light chain is described in SEQ ID NO: 72 (FIG. 81) of the Sequence Listing. A nucleotide sequence encoding the amino acid sequence of SEQ ID NO: 72 is described in SEQ ID NO: 71 (FIG. 80) of the Sequence Listing.

### 7)-2 Design of Humanized Form of Anti-GPRC5D Antibody 7B4

### 7)-2-1 Molecular Modeling of 7B4 Variable Regions

The molecular modeling of the 7B4 variable regions was carried out by a method generally known as homology modeling (Methods in Enzymology, 203, 121-153, (1991)). The variable regions of 7B4 determined above were compared with the primary sequences (three-dimensional structures derived from X-ray crystal structures are available) of human immunoglobulin variable regions registered in Protein Data Bank (Nuc. Acid Res. 35, D301-D303 (2007)). As a result, 1BGX was selected because it had the highest sequence identity to the heavy and light chain variable regions of 7B4. The three-dimensional structures of framework regions were prepared as a “framework model” by combining the coordinates of 1BGX corresponding to the heavy and light chains of 7B4. Subsequently, the typical conformation of each CDR was incorporated into the framework model. Finally, energy calculation for excluding disadvantageous interatomic contact was conducted in order to obtain possible molecular models of the 7B4 variable regions in terms of energy. These procedures were performed using a commercially available protein three-dimensional structure analysis program BioLuminate (Schrodinger, LLC).

7)-2-2 Design of Amino Acid Sequence of Humanized h7B4

Humanized h7B4 was constructed by a method generally known as CDR grafting (Proc. Natl. Acad. Sci. USA 86, 10029-10033 (1989)). An acceptor antibody was selected on the basis of the identity of amino acids in framework regions.

The sequences of the framework regions of 7B4 were compared with the framework regions of human subgroup consensus sequences or germline sequences specified by KABAT et al. (Sequences of Proteins of Immunological Interest, 5th Ed. Public Health Service National Institutes of Health, Bethesda, Md. (1991)). As a result, a human gamma chain subgroup 2 consensus sequence was selected as a heavy chain acceptor while a human kappa chain subgroup 3 consensus sequence was selected as light chain acceptor due to their high sequence identity as to framework regions. The amino acid residues of the framework regions of the acceptors were aligned with the amino acid residues of the 7B4 framework regions to identify the positions of amino acids that did not match there-between. The positions of these residues were analyzed using the three-dimensional model of 7B4 constructed above. Then, the donor residues to be grafted onto the acceptors were selected according to the criteria provided by Queen et al. (Proc. Natl. Acad. Sci. USA 86, 10029-10033 (1989)). Some donor residues thus selected were transferred to the acceptor antibody to construct the humanized h7B4 sequence as described in Examples below. The light chain was not limited by donor residues, and depending on a site, residues of a kappa chain subgroup 1 consensus sequence were transferred thereto.

### 7)-2-3 Humanization of 7B4 Heavy Chain

7)-2-3-1 Humanized h7B4_H1 Type Heavy Chain

A humanized h7B4 heavy chain designed from the chimeric c7B4 heavy chain shown in SEQ ID NO: 42 by the replacement in the variable region of glutamic acid at amino acid position 1 with glutamine, isoleucine at amino acid position 2 with valine, histidine at amino acid position 3 with glutamine, serine at amino acid position 17 with threonine, serine at amino acid position 23 with threonine, threonine at amino acid position 25 with serine, lysine at amino acid position 40 with glutamine, phenylalanine at amino acid position 41 with proline, asparagine at amino acid position 44 with lysine, lysine at amino acid position 45 with glycine, methionine at amino acid position 46 with leucine, methionine at amino acid position 49 with isoleucine, isoleucine at amino acid position 68 with valine, serine at amino acid position 69 with threonine, threonine at amino acid position 71 with serine, phenylalanine at amino acid position 80 with serine, glutamine at amino acid position 82 with lysine, asparagine at amino acid position 84 with serine, threonine at amino acid position 88 with alanine, glutamic acid at amino acid position 89 with alanine, threonine at amino acid position 93 with valine, alanine at amino acid position 117 with threonine, and serine at amino acid position 118 with leucine was designated as “humanized h7B4_H1 type heavy chain” (also referred to as “h7B4_H1”).

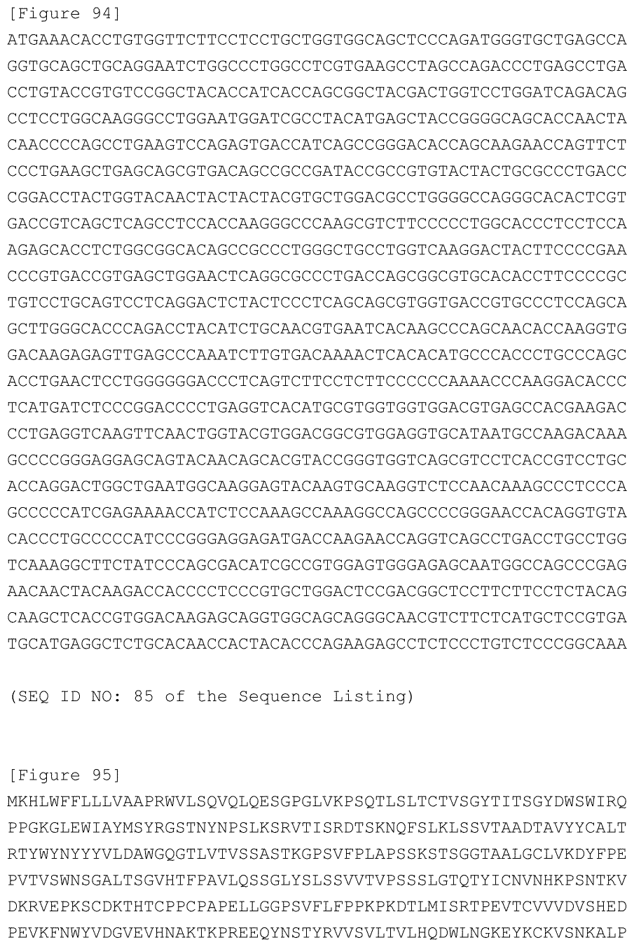

The amino acid sequence of the humanized h7B4_H1 type heavy chain is described in SEQ ID NO: 86 (FIG. 95) of the Sequence Listing. A nucleotide sequence encoding the amino acid sequence of SEQ ID NO: 86 is described in SEQ ID NO: 85 (FIG. 94) of the Sequence Listing.

7)-2-3-2 Humanized h7B4_H2 Type Heavy Chain

A humanized h7B4 heavy chain designed from the chimeric c7B4 heavy chain shown in SEQ ID NO: 42 by the replacement in the variable region of histidine at amino acid position 3 with glutamine, serine at amino acid position 17 with threonine, serine at amino acid position 23 with threonine, threonine at amino acid position 25 with serine, lysine at amino acid position 40 with glutamine, phenylalanine at amino acid position 41 with proline, asparagine at amino acid position 44 with lysine, lysine at amino acid position 45 with glycine, methionine at amino acid position 46 with leucine, methionine at amino acid position 49 with isoleucine, alanine at amino acid position 50 with glycine, isoleucine at amino acid position 68 with valine, serine at amino acid position 69 with threonine, threonine at amino acid position 71 with serine, phenylalanine at amino acid position 80 with serine, glutamine at amino acid position 82 with lysine, asparagine at amino acid position 84 with serine, threonine at amino acid position 88 with alanine, glutamic acid at amino acid position 89 with alanine, threonine at amino acid position 93 with valine, alanine at amino acid position 117 with threonine, and serine at amino acid position 118 with leucine was designated as “humanized h7B4_H2 type heavy chain” (also referred to as “h7B4_H2”).

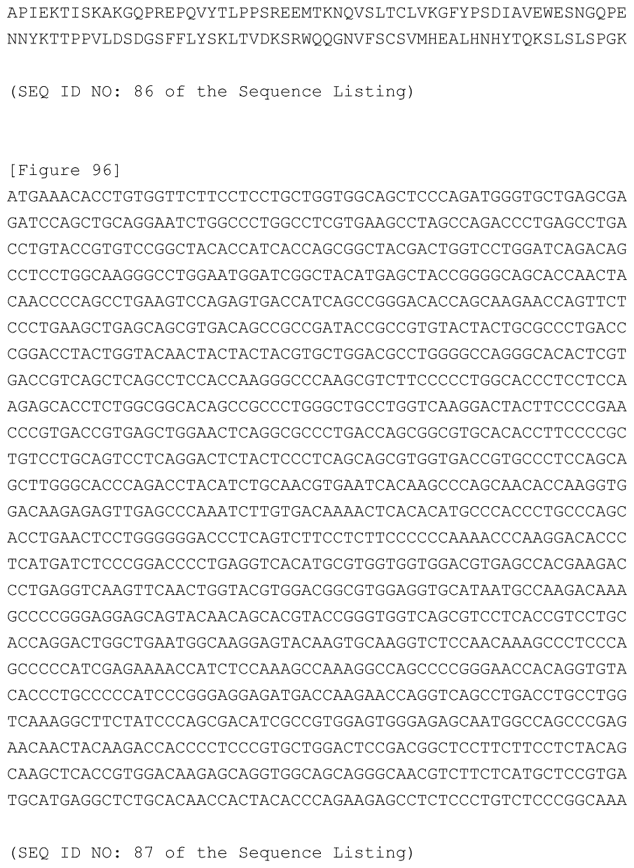

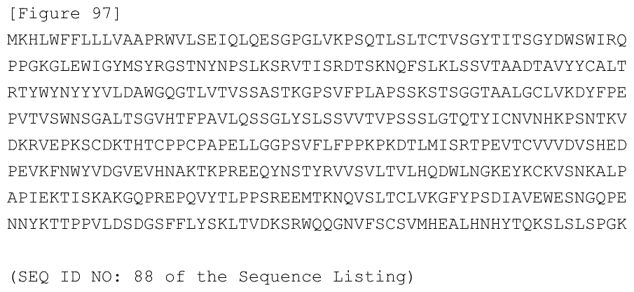

The amino acid sequence of the humanized h7B4_H2 type heavy chain is described in SEQ ID NO: 88 (FIG. 97) of the Sequence Listing. A nucleotide sequence encoding the amino acid sequence of SEQ ID NO: 88 is described in SEQ ID NO: 87 (FIG. 96) of the Sequence Listing.

7)-2-3-3 Humanized h7B4_H3 Type Heavy Chain

A humanized h7B4 heavy chain designed from the chimeric c7B4 heavy chain shown in SEQ ID NO: 42 by the replacement in the variable region of histidine at amino acid position 3 with glutamine, serine at amino acid position 17 with threonine, serine at amino acid position 23 with threonine, threonine at amino acid position 25 with serine, lysine at amino acid position 40 with glutamine, phenylalanine at amino acid position 41 with proline, asparagine at amino acid position 44 with lysine, lysine at amino acid position 45 with glycine, methionine at amino acid position 46 with leucine, methionine at amino acid position 49 with isoleucine, isoleucine at amino acid position 68 with valine, serine at amino acid position 69 with threonine, threonine at amino acid position 71 with serine, phenylalanine at amino acid position 80 with serine, glutamine at amino acid position 82 with lysine, asparagine at amino acid position 84 with serine, threonine at amino acid position 88 with alanine, glutamic acid at amino acid position 89 with alanine, threonine at amino acid position 93 with valine, alanine at amino acid position 117 with threonine, and serine at amino acid position 118 with leucine was designated as “humanized h7B4_H3 type heavy chain” (also referred to as “h7B4_H3”).

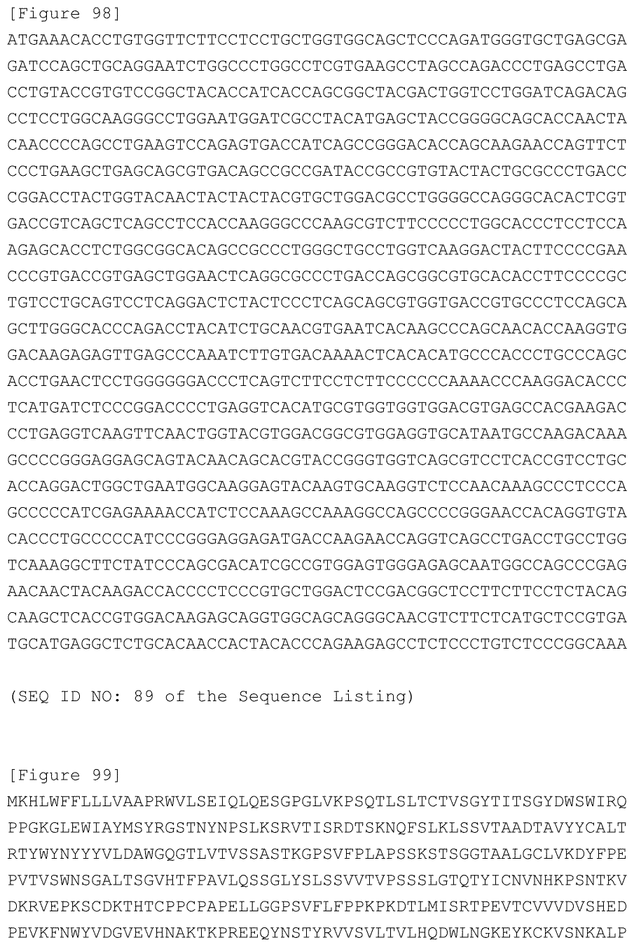

The amino acid sequence of the humanized h7B4_H3 type heavy chain is described in SEQ ID NO: 90 (FIG. 99) of the Sequence Listing. A nucleotide sequence encoding the amino acid sequence of SEQ ID NO: 90 is described in SEQ ID NO: 89 (FIG. 98) of the Sequence Listing.

7)-2-3-4 Humanized h7B4_H5 Type Heavy Chain

A humanized h7B4 heavy chain designed from the chimeric c7B4 heavy chain shown in SEQ ID NO: 42 by the replacement in the variable region of histidine at amino acid position 3 with glutamine, serine at amino acid position 17 with threonine, serine at amino acid position 23 with threonine, threonine at amino acid position 25 with serine, phenylalanine at amino acid position 41 with proline, methionine at amino acid position 49 with isoleucine, isoleucine at amino acid position 68 with valine, serine at amino acid position 69 with threonine, threonine at amino acid position 71 with serine, phenylalanine at amino acid position 80 with serine, glutamine at amino acid position 82 with lysine, asparagine at amino acid position 84 with serine, threonine at amino acid position 88 with alanine, glutamic acid at amino acid position 89 with alanine, threonine at amino acid position 93 with valine, alanine at amino acid position 117 with threonine, and serine at amino acid position 118 with leucine was designated as “humanized h7B4_H5 type heavy chain” (also referred to as “h7B4_H5”).

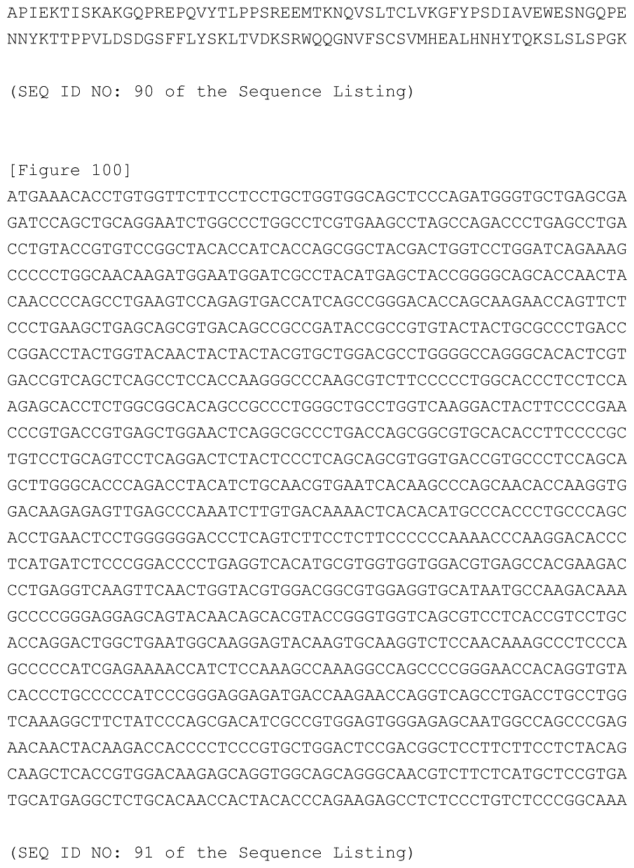

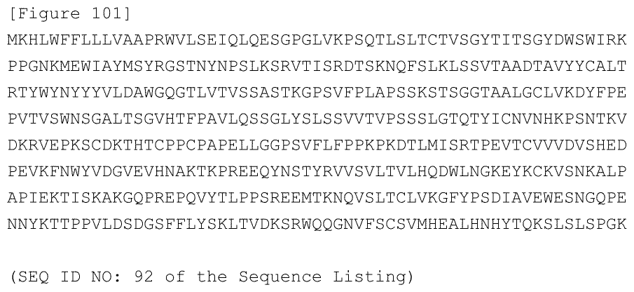

The amino acid sequence of the humanized h7B4_H5 type heavy chain is described in SEQ ID NO: 92 (FIG. 101) of the Sequence Listing. A nucleotide sequence encoding the amino acid sequence of SEQ ID NO: 92 is described in SEQ ID NO: 91 (FIG. 100) of the Sequence Listing.

### 7)-2-4 Humanization of 7B4 Light Chain

7)-2-4-1 Humanized h7B4_L1 Type Light Chain

A humanized h7B4 light chain designed from the chimeric c7B4 light chain shown in SEQ ID NO: 38 by the replacement in the variable region of aspartic acid at amino acid position 1 with glutamic acid, glutamine at amino acid position 3 with valine, methionine at amino acid position 4 with leucine, serine at amino acid position 9 with glycine, phenylalanine at amino acid position 10 with threonine, alanine at amino acid position 13 with leucine, valine at amino acid position 15 with proline, valine at amino acid position 19 with alanine, leucine at amino acid position 40 with proline, glutamic acid at amino acid position 42 with glutamine, lysine at amino acid position 45 with arginine, serine at amino acid position 60 with aspartic acid, glycine at amino acid position 77 with arginine, glutamine at amino acid position 79 with glutamic acid, valine at amino acid position 83 with phenylalanine, threonine at amino acid position 85 with valine, phenylalanine at amino acid position 87 with tyrosine, alanine at amino acid position 99 with glutamine, leucine at amino acid position 103 with valine, and leucine at amino acid position 105 with isoleucine was designated as “humanized h7B4_L1 type light chain” (also referred to as “h7B4_L1”).

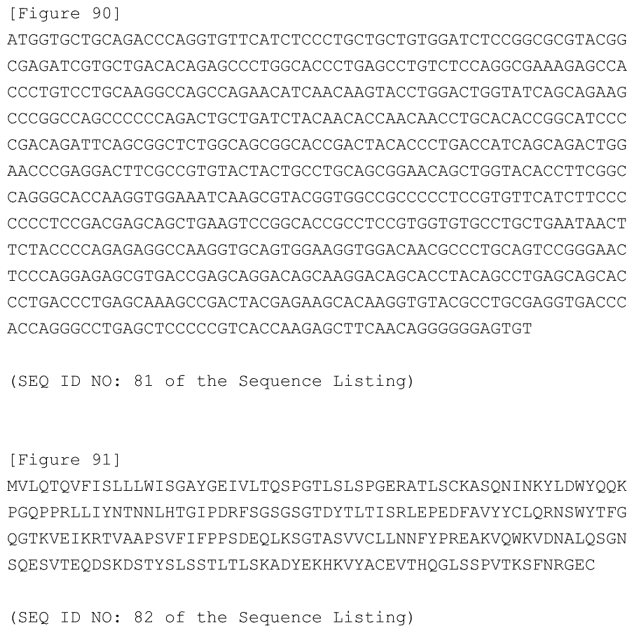

The amino acid sequence of the humanized h7B4_L1 type light chain is described in SEQ ID NO: 82 (FIG. 91) of the Sequence Listing. A nucleotide sequence encoding the amino acid sequence of SEQ ID NO: 82 is described in SEQ ID NO: 81 (FIG. 90) of the Sequence Listing.

7)-2-4-2 Humanized h7B4_L2 Type Light Chain

A humanized h7B4 light chain designed from the chimeric c7B4 light chain shown in SEQ ID NO: 38 by the replacement in the variable region of phenylalanine at amino acid position 10 with serine, alanine at amino acid position 13 with leucine, valine at amino acid position 15 with proline, valine at amino acid position 19 with alanine, leucine at amino acid position 40 with proline, glutamic acid at amino acid position 42 with glutamine, lysine at amino acid position 45 with arginine, serine at amino acid position 60 with aspartic acid, glycine at amino acid position 77 with arginine, glutamine at amino acid position 79 with glutamic acid, valine at amino acid position 83 with phenylalanine, threonine at amino acid position 85 with valine, phenylalanine at amino acid position 87 with tyrosine, alanine at amino acid position 99 with glutamine, leucine at amino acid position 103 with valine, and leucine at amino acid position 105 with isoleucine was designated as “humanized h7B4_L2 type light chain” (also referred to as “h7B4_L2”).

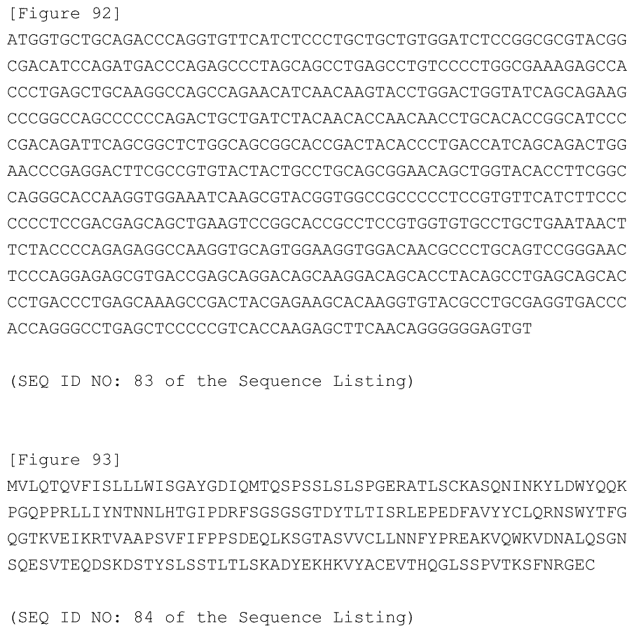

The amino acid sequence of the humanized h7B4_L2 type light chain is described in SEQ ID NO: 84 (FIG. 93) of the Sequence Listing. A nucleotide sequence encoding the amino acid sequence of SEQ ID NO: 84 is described in SEQ ID NO: 83 (FIG. 92) of the Sequence Listing.

### Example 8

### Construction of Expression Vectors for Humanized Antibodies (h2B1 and h7B4) of Rat Anti-Human GPRC5D Antibodies (2B1 and 7B4) and Preparation of Antibodies

8)-1 Construction of h2B1 Heavy Chain Expression Vector
8)-1-1 Construction of h2B1_H1 Type Heavy Chain

A DNA fragment comprising the h2B1_H1 variable region-encoding DNA sequence represented by nucleotide positions 58 to 426 of the h2B1_H1 nucleotide sequence represented by SEQ ID NO: 73 was synthesized (GeneArt Artificial Gene Synthesis Service). A h2B1_H1 expression vector was constructed in the same way as in Example 8)-1-2. The obtained expression vector was designated as “pCMA/h2B1_H1”.

8)-1-2 Construction of h2B1_H2 Type Heavy Chain

A DNA fragment comprising the h2B1_H2 variable region-encoding DNA sequence represented by nucleotide positions 58 to 426 of the h2B1_H2 nucleotide sequence represented by SEQ ID NO: 75 was synthesized (GeneArt Artificial Gene Synthesis Service). The synthesized DNA fragment was amplified by PCR and inserted to the restriction enzyme B1pI-cleaved site of the chimeric and humanized antibody heavy chain expression vector pCMA-G1 using In-Fusion HD PCR cloning kit (Clontech Laboratories, Inc.) to construct a h2B1_H2 expression vector. The obtained expression vector was designated as “pCMA/h2B1_H2”.

8)-1-3 Construction of h2B1_H3 Type Heavy Chain

A DNA fragment comprising the h2B1_H3 variable region-encoding DNA sequence represented by nucleotide positions 58 to 426 of the h2B1_H3 nucleotide sequence represented by SEQ ID NO: 77 was synthesized (GeneArt Artificial Gene Synthesis Service). A h2B1_H3 expression vector was constructed in the same way as in Example 8)-1-2. The obtained expression vector was designated as “pCMA/h2B1_H3”.

8)-1-4 Construction of h2B1_H4 Type Heavy Chain

A DNA fragment comprising the h2B1_H4 variable region-encoding DNA sequence represented by nucleotide positions 58 to 426 of the h2B1_H4 nucleotide sequence represented by SEQ ID NO: 79 was synthesized (GeneArt Artificial Gene Synthesis Service). A h2B1_H4 expression vector was constructed in the same way as in Example 8)-1-2. The obtained expression vector was designated as “pCMA/h2B1_H4”.

8)-2 Construction of h2B1 Light Chain Expression Vector
8)-2-1 Construction of h2B1_L1 Type Light Chain

A DNA fragment comprising the h2B1_L1 variable region-encoding DNA sequence represented by nucleotide positions 61 to 381 of the h2B1_L1 nucleotide sequence represented by SEQ ID NO: 63 was synthesized (GeneArt Gene Synthesis Service). The synthesized DNA fragment was amplified by PCR and inserted to the restriction enzyme BsiWI-cleaved site of the chimeric and humanized antibody light chain expression vector pCMA-LK using In-Fusion HD PCR cloning kit (Clontech Laboratories, Inc.) to construct a h2B1_L1 expression vector. The obtained expression vector was designated as “pCMA/h2B1_L1”.

8)-2-2 Construction of h2B1_L2 Type Light Chain

A DNA fragment comprising the h2B1_L2 variable region-encoding DNA sequence represented by nucleotide positions 61 to 381 of the h2B1_L2 nucleotide sequence represented by SEQ ID NO: 65 was synthesized (GeneArt Gene Synthesis Service). A h2B1_L2 expression vector was constructed in the same way as in Example 8)-2-1. The obtained expression vector was designated as “pCMA/h2B1_L2”.

8)-2-3 Construction of h2B1_L3 Type Light Chain

A DNA fragment comprising the h2B1_L3 variable region-encoding DNA sequence represented by nucleotide positions 61 to 381 of the h2B1_L3 nucleotide sequence represented by SEQ ID NO: 67 was synthesized (GeneArt Gene Synthesis Service). A h2B1_L3 expression vector was constructed in the same way as in Example 8)-2-1. The obtained expression vector was designated as “pCMA/h2B1_L3”.

8)-2-4 Construction of h2B1_L4 Type Light Chain

A DNA fragment comprising the h2B1_L4 variable region-encoding DNA sequence represented by nucleotide positions 61 to 381 of the h2B1_L4 nucleotide sequence represented by SEQ ID NO: 69 was synthesized (GeneArt Gene Synthesis Service). A h2B1_L4 expression vector was constructed in the same way as in Example 8)-2-1. The obtained expression vector was designated as “pCMA/h2B1_L4”.

8)-2-5 Construction of h2B1_L5 Type Light Chain

A DNA fragment comprising the h2B1_L5 variable region-encoding DNA sequence represented by nucleotide positions 61 to 381 of the h2B1_L5 nucleotide sequence represented by SEQ ID NO: 71 was synthesized (GeneArt Gene Synthesis Service). A h2B1_L5 expression vector was constructed in the same way as in Example 8)-2-1. The obtained expression vector was designated as “pCMA/h2B1_L5”.

8)-3 Construction of h7B4 Heavy Chain Expression Vector
8)-3-1 Construction of h7B4_H1 Type Heavy Chain

A DNA fragment comprising the h7B4_H1 variable region-encoding DNA sequence represented by nucleotide positions 58 to 426 of the h7B4_H1 nucleotide sequence represented by SEQ ID NO: 85 was synthesized (GeneArt Artificial Gene Synthesis Service). A h7B4_H1 expression vector was constructed in the same way as in Example 8)-1-1. The obtained expression vector was designated as “pCMA/h7B4_H1”.

8)-3-2 Construction of h7B4_H2 Type Heavy Chain

A DNA fragment comprising the h7B4_H2 variable region-encoding DNA sequence represented by nucleotide positions 58 to 426 of the h7B4_H2 nucleotide sequence represented by SEQ ID NO: 87 was synthesized (GeneArt Artificial Gene Synthesis Service). A h7B4_H2 expression vector was constructed in the same way as in Example 8)-1-1. The obtained expression vector was designated as “pCMA/h7B4_H2”.

8)-3-3 Construction of h7B4_H3 Type Heavy Chain

A DNA fragment comprising the h7B4_H3 variable region-encoding DNA sequence represented by nucleotide positions 58 to 426 of the h7B4_H3 nucleotide sequence represented by SEQ ID NO: 89 was synthesized (GeneArt Artificial Gene Synthesis Service). A h7B4_H3 expression vector was constructed in the same way as in Example 8)-1-1. The obtained expression vector was designated as “pCMA/h7B4_H3”.

8)-3-4 Construction of h7B4_H5 Type Heavy Chain

A DNA fragment comprising the h7B4_H5 variable region-encoding DNA sequence represented by nucleotide positions 58 to 426 of the h7B4_H5 nucleotide sequence represented by SEQ ID NO: 91 was synthesized (GeneArt Artificial Gene Synthesis Service). A h7B4_H5 expression vector was constructed in the same way as in Example 8)-1-1. The obtained expression vector was designated as “pCMA/h7B4_H5”.

8)-4 Construction of h7B4 Light Chain Expression Vector
8)-4-1 Construction of h7B4_L1 Type Light Chain

A DNA fragment comprising the h7B4_L1 variable region-encoding DNA sequence represented by nucleotide positions 61 to 378 of the h7B4_L1 nucleotide sequence represented by SEQ ID NO: 81 was synthesized (GeneArt Gene Synthesis Service). A h7B4_L1 expression vector was constructed in the same way as in Example 8)-2-1. The obtained expression vector was designated as “pCMA/h7B4_L1”.

8)-4-2 Construction of h7B4_L2 Type Light Chain

A DNA fragment comprising the h7B4_L2 variable region-encoding DNA sequence represented by nucleotide positions 61 to 378 of the h7B4_L2 nucleotide sequence represented by SEQ ID NO: 83 was synthesized (GeneArt Gene Synthesis Service). A h7B4_L2 expression vector was constructed in the same way as in Example 8)-2-1. The obtained expression vector was designated as “pCMA/h7B4_L2”.

8)-5 Preparation of Humanized Antibodies (h2B1 and h7B4) (FreeStyle 293F Cells)
8)-5-1 Small-Scale Production of Humanized Antibodies (h2B1 and h7B4)

FreeStyle 293F cells (Invitrogen Corp.) were subcultured and cultured according to the manual.

1×10⁷ FreeStyle 293F cells (Invitrogen Corp.) in the logarithmic growth phase were diluted to 9.6 mL with FreeStyle 293 expression medium (Invitrogen Corp.), then inoculated to 30 mL Square Storage Bottle (Nalgene/Thermo Fisher Scientific Inc.), and shake-cultured at 90 rpm at 37° C. for 1 hour in an 8% CO₂ incubator. 30 μg of polyethyleneimine (Polysciences #24765) was dissolved in 200 μL of Opti-Pro SFM (Invitrogen Corp.). Next, each heavy chain expression vector ((4 μg) and light chain expression vector (6 μg) prepared using PureLink HiPure Plasmid kit (Invitrogen Corp.) were added to 200 μL of Opti-Pro SFM (Invitrogen Corp.). 200 μL of the expression vector/Opti-Pro SFM mixed solution was added to 200 μL of the polyethyleneimine/Opti-Pro SFM mixed solution, and the mixture was gently stirred, further left for 5 minutes, and then added to the FreeStyle 293F cells. The cells were shake-cultured at 90 rpm at 37° C. for 7 days in an 8% CO₂ incubator, and the obtained culture supernatant was filtered through Minisart-Plus filter (Sartorius Japan K.K.) and used as a sample for evaluation.

h2B1_H1/L1 was obtained by the combination of pCMA/h2B1_H1 and pCMA/h2B1 L1. h2B1_H1/L2 was obtained by the combination of pCMA/h2B1_H1 and pCMA/h2B1_L2. h2B1_H2/L2 was obtained by the combination of pCMA/h2B1_H2 and pCMA/h2B1_L2. h2B1_H2/L3 was obtained by the combination of pCMA/h2B1_H2 and pCMA/h2B1_L3. h2B1_H2/L4 was obtained by the combination of pCMA/h2B1_H2 and pCMA/h2B1_L4. h2B1_H2/L5 was obtained by the combination of pCMA/h2B1_H2 and pCMA/h2B1_L5. h2B1_H3/L3 was obtained by the combination of pCMA/h2B1_H3 and pCMA/h2B1_L3. h2B1_H3/L4 was obtained by the combination of pCMA/h2B1_H3 and pCMA/h2B1_L4. h2B1_H3/L5 was obtained by the combination of pCMA/h2B1_H3 and pCMA/h2B1_L5. h2B1_H4/L1 was obtained by the combination of pCMA/h2B1_H4 and pCMA/h2B1 L1. h2B1_H4/L3 was obtained by the combination of pCMA/h2B1_H4 and pCMA/h2B1_L3. h2B1_H4/L4 was obtained by the combination of pCMA/h2B1_H4 and pCMA/h2B1_L4. h2B1_H4/L5 was obtained by the combination of pCMA/h2B1_H4 and pCMA/h2B1_L5. h7B4_H1/L2 was obtained by the combination of pCMA/h7B4_H1 and pCMA/h7B4_L2. h7B4_H2/L2 was obtained by the combination of pCMA/h7B4_H2 and pCMA/h7B4_L2. h7B4_H3/L1 was obtained by the combination of pCMA/h7B4_H3 and pCMA/h7B4_L1. h7B4_H3/L2 was obtained by the combination of pCMA/h7B4_H3 and pCMA/h7B4_L2. h7B4_H5/L1 was obtained by the combination of pCMA/h7B4_H5 and pCMA/h7B4_L1.

8)-5-2 Production of Humanized Antibodies (h2B1 and h7B4)

Humanized antibodies were produced in the same way as in Example 4)-9-1. Specifically, h2B1_H1/L1 was obtained by the combination of pCMA/h2B1_H1 and pCMA/h2B1_L1. h2B1_H2/L5 was obtained by the combination of pCMA/h2B1_H2 and pCMA/h2B1_L5. h2B1_H4/L5 was obtained by the combination of pCMA/h2B1_H4 and pCMA/h2B1_L5. h7B4_H1/L2 was obtained by the combination of pCMA/h7B4_H1 and pCMA/h7B4_L2. h7B4_H3/L1 was obtained by the combination of pCMA/h7B4_H3 and pCMA/h7B4_L1.

8)-5-3 Purification of Humanized Antibodies (h2B1 and h7B4)

Each antibody was purified from the culture supernatant obtained in Example 8)-5-2 by two steps using rProtein A affinity chromatography (at 4 to 6° C.) and ceramic hydroxyapatite (at room temperature). Buffer replacement steps after the rProtein A affinity chromatography purification and after the ceramic hydroxyapatite purification were carried out at 4 to 6° C. The culture supernatant was applied to MabSelect SuRe (manufactured by GE Healthcare Bio-Sciences Corp., HiTrap column) equilibrated with PBS. After entry of the whole culture supernatant in the column, the column was washed with PBS in an amount at least twice the column volume. Next, antibody-containing fractions were collected by elution with a 2 M arginine hydrochloride solution (pH 4.0). The fractions were buffer-replaced with PBS by dialysis (Thermo Fisher Scientific Inc., Slide-A-Lyzer Dialysis Cassette) and then diluted 5-fold with a buffer of 5 mM sodium phosphate and 50 mM MES (pH 7.0). The resulting antibody solution was applied to a ceramic hydroxyapatite column (Bio-Rad Laboratories, Inc., Bio-Scale CHT Type-1 Hydroxyapatite Column) equilibrated with a buffer of 5 mM NaPi, 50 mM MES, and 30 mM NaCl (pH 7.0). Antibody-containing fractions were collected by linear concentration gradient elution using sodium chloride. The fractions were buffer-replaced with HBSor (25 mM histidine and 5% sorbitol, pH 6.0) by dialysis (Thermo Fisher Scientific Inc., Slide-A-Lyzer Dialysis Cassette). The fractions were concentrated and adjusted to an IgG concentration of 10 mg/ml or higher using Centrifugal UF Filter Device VIVASPIN 20 (molecular weight cutoff: UF10K, Sartorius Japan K.K., at 4° C.). Finally, the antibody solution was filtered through Minisart-Plus filter (Sartorius Japan K.K.) and used as a purified sample.

### Example 9

### In Vitro Activity Evaluation of Humanized Anti-GPRC5D Antibody

9)-1 Evaluation of Binding Activity of Humanized Anti-GPRC5D Antibodies (h2B1_H1/L1 to h2B1_H4/L5 and h7B4_H1/L2 to h7B4_H5/L1) Against Human GPRC5D by Flow Cytometry

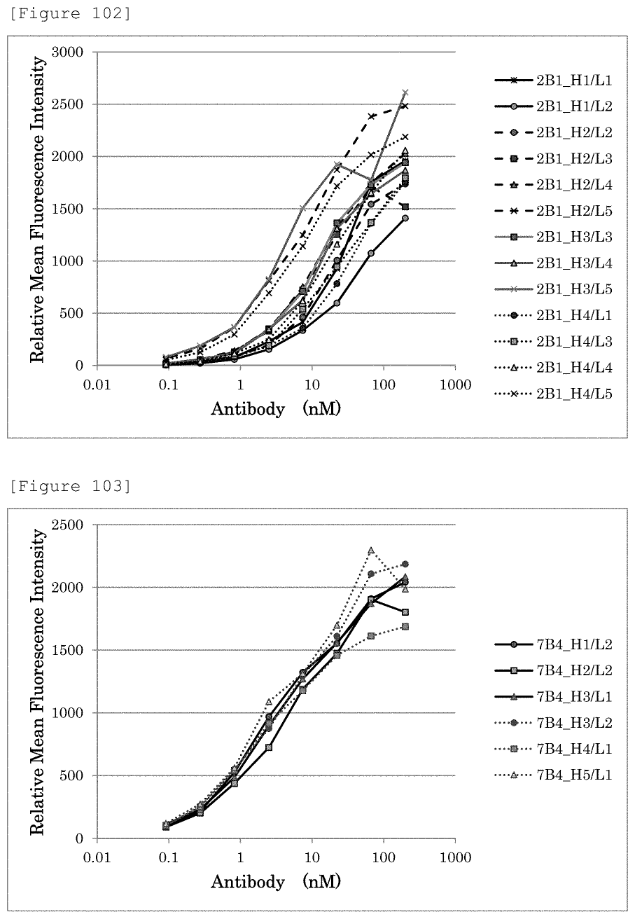

Human multiple myeloma cell line KHM-1B cells expressing GPRC5D were adjusted to a concentration of 2×10⁶ cells/mL with PBS containing 5% FBS, inoculated in an amount of 100 μL/well to a 96-well U-bottomed microplate, and centrifuged to remove a supernatant. The culture supernatant of each humanized anti-GPRC5D antibody obtained in Example 8)-3-1 or Human IgG isotype control antibody (Calbiochem/Merck Millipore Corp.) adjusted to 14 ng/mL to 30 μg/mL was added thereto in an amount of 100 μL/well, and the plate was left standing at 4° C. for 1 hour. The cells were washed twice with PBS containing 5% FBS. Then, R-Phycoerythrin AffiniPure F(ab′)2 Fragment Goat Anti-Human IgG, Fcγ Fragment Specific (Jackson ImmunoResearch Laboratories, Inc.) diluted 100-fold with PBS containing 5% FBS was added thereto in an amount of 100 μL/well, and the plate was left standing at 4° C. for 1 hour. The cells were washed twice with PBS containing 5% FBS and then resuspended in PBS containing 5% FBS, followed by detection using a flow cytometer (FACSCanto™ II; Becton, Dickinson and Company). The data was analyzed using Flowjo (Tree Star, Inc.). The PE fluorescence intensity of the cell fraction was plotted to a histogram, and the mean fluorescence intensity (MFI) was calculated. The MFI value of the control antibody was subtracted from the MFI value of the GPRC5D antibody to calculate a relative value of MFI (rMFI). FIG. 102 shows the results about the humanized anti-GPRC5D antibodies h2B1_H1/L1 to h2B1_H4/L5, and FIG. 103 shows the results about the humanized anti-GPRC5D antibodies h7B4_H1/L2 to h7B4_H5/L1. As shown in FIGS. 102 and 103, these humanized anti-GPRC5D antibodies were found to bind to human GPRC5D.

9)-2 Evaluation of Binding Activity of Humanized Anti-GPRC5D Antibodies (h2B1_H1/L1 to h2B1_H4/L5 and h7B4_H1/L2 to h7B4_H5/L1) Against Cynomolgus Monkey GPRC5D by Flow Cytometry

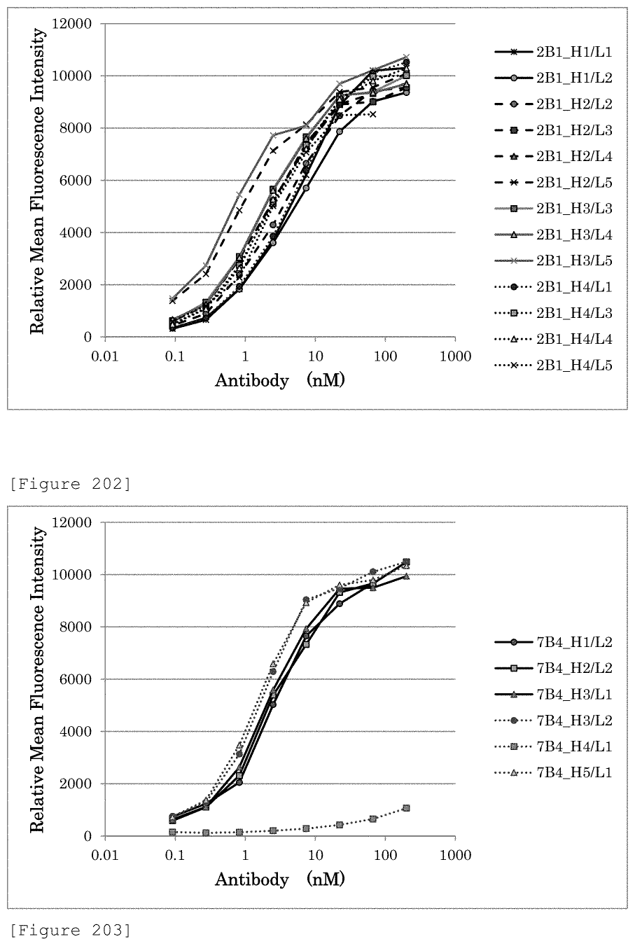

Staining and analysis were carried out in the same way as in Example 9)-1 using the KMS-11_cGPRC5D cells prepared in Example 5)-2-2. As shown in FIGS. 201 and 202, these humanized anti-GPRC5D antibodies were found to bind to cynomolgus monkey GPRC5D.

9)-3 ADCC Activity Evaluation of Humanized Anti-GPRC5D Antibodies (h2B1_H1/L1, h2B1_H2/L5, h2B1_H4/L5, h7B4_H1/L2, and h7B4_H3/L1)

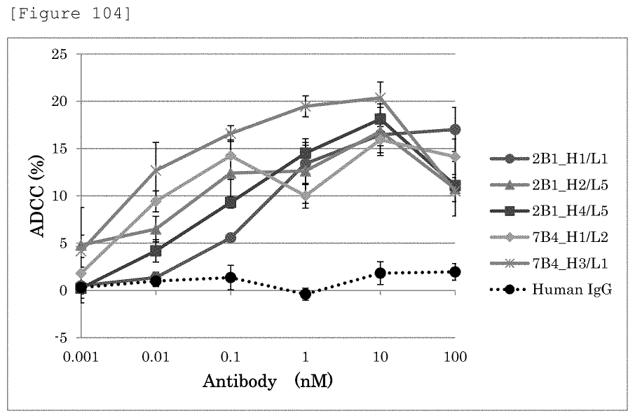

The KHM-1B cells prepared in Example 2)-3-1 were added in an amount of 50 μL/well to a 96-well U-bottomed microplate. Each humanized anti-GPRC5D antibody (h2B1_H1/L1, h2B1_H2/L5, h2B1_H4/L5, h7B4_H1/L2 and h7B4_H3/L1) or human control antibody (hIgG1) (Calbiochem/Merck Millipore Corp.) adjusted to 0.15 ng/mL to 15 μg/mL (final concentration) was added thereto in an amount of 50 μL/well, and the plate was left standing at 4° C. for 30 minutes. The effector cells prepared in Example 1)-5-3 were further added thereto in an amount of 100 μL/well. After centrifugation at room temperature at 1200 rpm for 3 minutes, the cells were cultured at 37° C. for 4 hours under 5% CO₂ conditions. A 50 μL aliquot of the supernatant was recovered into LumaPlate (PerkinElmer, Inc.) and dried overnight at 50° C., followed by measurement using a plate reader (TopCount; PerkinElmer, Inc.). The percentage of cells lysed by ADCC activity was calculated according to Example 1)-5-5. As shown in FIG. 104, h2B1_H1/L1, h2B1_H2/L5, h2B1_H4/L5, h7B4_H1/L2, and h7B4_H3/L1 were found to have ADCC activity.

### Example 10

### Obtainment of Anti-GPRC5D Antibody Derived from Human Antibody Phage Library, and Binding Activity Evaluation

10)-1 Isolation of scFv Having GPRC5D Binding Activity

scFv binding to human GPRC5D and cynomolgus monkey GPRC5D was isolated from a human antibody phage library. Phages were added to Dynabeads Streptavidin M-280 (Thermo Fisher Scientific Inc.) on which the amino-terminal peptide (synthesized by Peptide Institute, Inc.) of human (SEQ ID NO: 2 of the Sequence Listing; FIG. 3) or cynomolgus monkey GPRC5D having a biotinylated carboxy terminus was immobilized. Unbound phages were removed by washing operation using a magnet stand (DynaMag-2, Thermo Fisher Scientific Inc.). The amino-terminal peptide of cynomolgus monkey GPRC5D used had the following sequence:
Amino-terminal peptide of cynomolgus monkey GPRC5D:

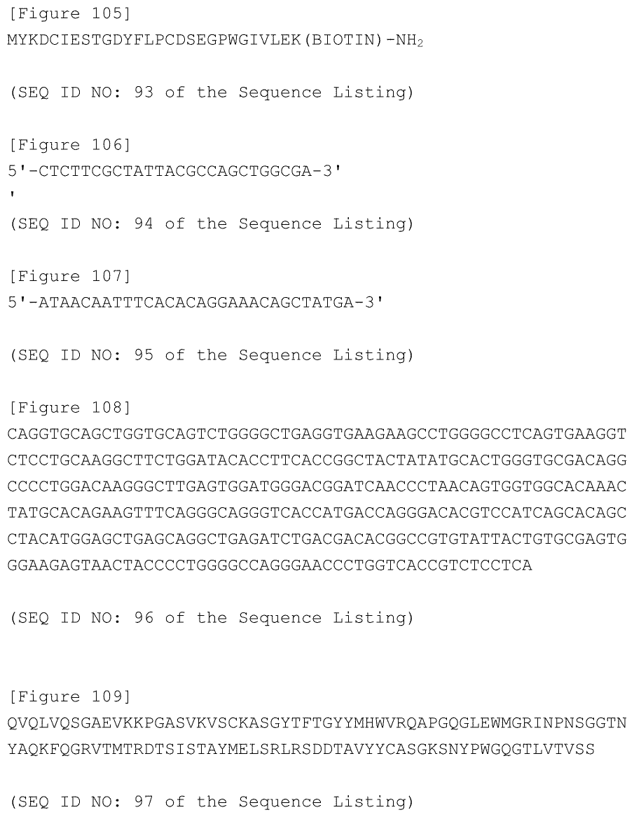

MYKDCIESTGDYFLPCDSEGPWGIVLEK(Biotin)-NH₂
(SEQ ID NO: 93 of the Sequence Listing; FIG. 105)

Then, E. coli (XL-1 Blue, Agilent Technologies, Inc.) was infected by the phages bound with the GPRC5D amino-terminal peptide, and the phages bound with the GPRC5D amino-terminal peptide were recovered and amplified. Alternatively, phages were added to Expi293F cells (Thermo Fisher Scientific Inc.) caused to transiently express human or cynomolgus monkey GPRC5D using the GPRC5D expression vector prepared in Example 1)-1-1 or 5)-2-1. Unbound phages were removed by washing operation. Then, E. coli was infected by the phages bound with the GPRC5D amino-terminal peptide, and the phages bound with the GPRC5D amino-terminal peptide were recovered and amplified. A total of 3 rounds of panning were carried out for the peptide or the Expi293F cells caused to transiently express human or cynomolgus monkey GPRC5D. After transfer from the polyclonal phagemid to an expression vector for E. coli to add FLAG and His tags to the carboxyl terminus of scFv, E. coli was transformed with the expression vector, and scFv was expressed in the presence of IPTG (isopropyl-β-D-thiogalactopyranoside) (Sigma-Aldrich Corp.) and subjected to screening by ELISA.

10)-2 Screening for GPRC5D-Binding scFv by ELISA

NeutrAvidin (Life Technologies Corp.) diluted to 1 μg/mL with PBS (0.01 M phosphate-buffered saline (pH 7.4) containing 0.138 M sodium chloride and 0.0027 M potassium chloride, Sigma-Aldrich Corp.) was added in an amount of 50 μL/well to 384-well Maxi-sorp plate (Black, Nunc/Thermo Fisher Scientific Inc.), and the plate was left standing overnight at 4° C. for immobilization. After washing three times with PBS containing 0.05% Tween-20 (Bio-Rad Laboratories, Inc.) (ELISA buffer), the amino-terminal peptide of the biotinylated human or cynomolgus monkey GPRC5D (also used in Example 10)-1) diluted to 1 μg/mL with PBS was added thereto, and the plate was shaken at room temperature for 1 hour. After washing three times with an ELISA buffer, the plate was blocked with Blocker Casein (Thermo Fisher Scientific Inc.) and washed three times with an ELISA buffer. Then, the culture solution of the scFv-expressing E. coli was added to the plate and reacted at room temperature for 2 hours. After washing three times with an ELISA buffer, a horseradish peroxidase (HRP)-labeled anti-FLAG antibody (Sigma-Aldrich Corp.) diluted 5000-fold with an ELISA buffer was added thereto in an amount of 50 μL/well and reacted at room temperature for 1 hour. After washing five times with ELISA buffer, SuperSignal Pico ELISA Chemiluminescent substrate (Thermo Fisher Scientific Inc.) was added. After 10 minutes, the chemiluminescence was measured using a plate reader (Envision 2104 Multilabel Reader, PerkinElmer, Inc.), and GPRC5D-binding scFv was selected.

### 10)-3 Sequencing of ELISA Positive Clone

The nucleotide sequences of the heavy and light chain variable regions of ELISA positive clones (C2037, C3048, C3015, and C3022) were analyzed by the dye terminator method (BigDye® Terminator v3.1, Thermo Fisher Scientific Inc.). The primer sequences used in the sequence analysis were as follows:

Primer A: 5′-CTCTTCGCTATTACGCCAGCTGGCGA-3′
(SEQ ID NO: 94 of the Sequence Listing; FIG. 106)
Primer B: 5′-ATAACAATTTCACACAGGAAACAGCTATGA-3′
(SEQ ID NO: 95 of the Sequence Listing; FIG. 107)

The variable region-encoding nucleotide sequences of the genes of the C2037 antibody, the C3048 antibody, the C3015 antibody, and the C3022 antibody were determined by the analysis.

The determined nucleotide sequence of the cDNA encoding the heavy chain variable region of C2037 is shown in SEQ ID NO: 96 (FIG. 108), and the amino acid sequence thereof is shown in SEQ ID NO: 97 (FIG. 109).

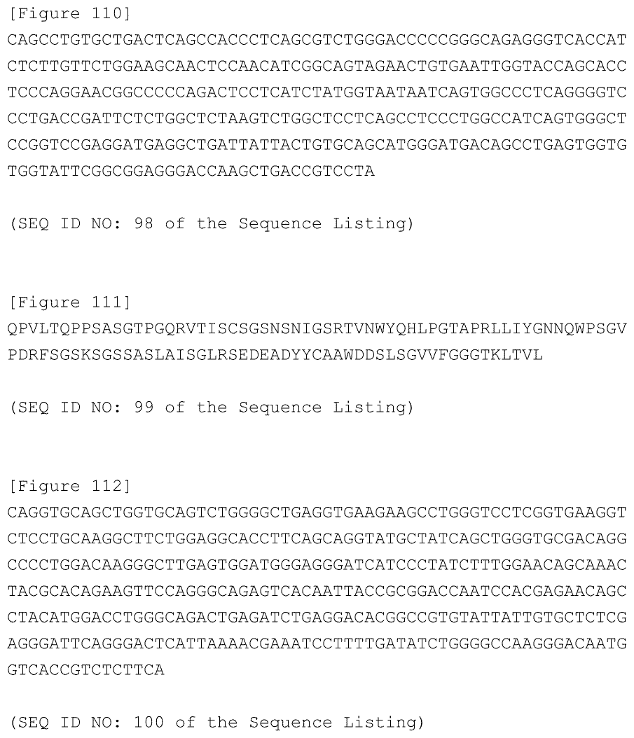

The determined nucleotide sequence of the cDNA encoding the light chain variable region of C2037 is shown in SEQ ID NO: 98 (FIG. 110), and the amino acid sequence thereof is shown in SEQ ID NO: 99 (FIG. 111).

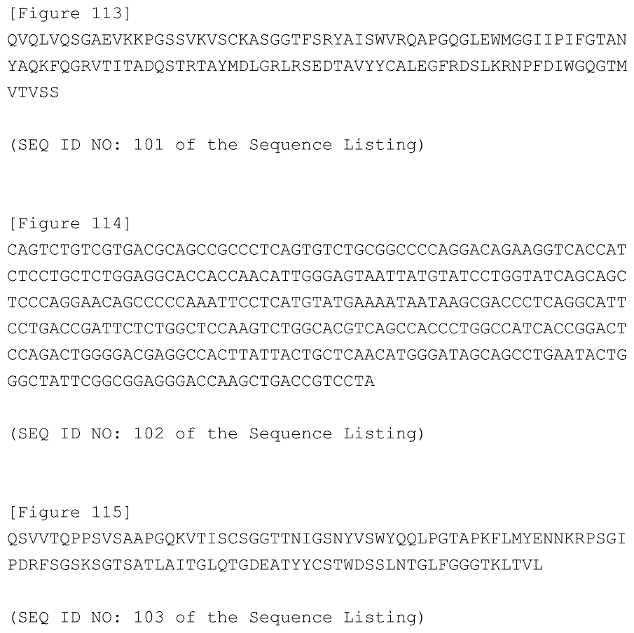

The determined nucleotide sequence of the cDNA encoding the heavy chain variable region of C3048 is shown in SEQ ID NO: 100 (FIG. 112), and the amino acid sequence thereof is shown in SEQ ID NO: 101 (FIG. 113).

The determined nucleotide sequence of the cDNA encoding the light chain variable region of C3048 is shown in SEQ ID NO: 102 (FIG. 114), and the amino acid sequence thereof is shown in SEQ ID NO: 103 (FIG. 115).

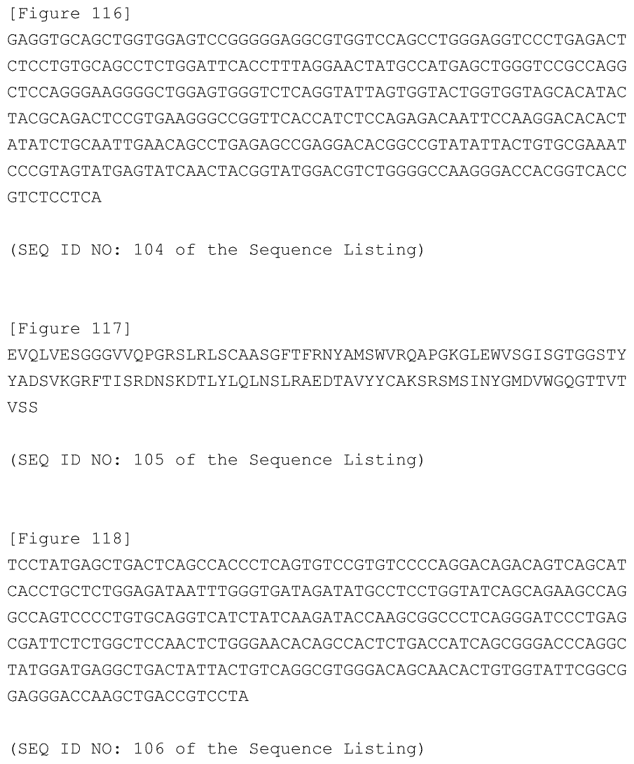

The determined nucleotide sequence of the cDNA encoding the heavy chain variable region of C3015 is shown in SEQ ID NO: 104 (FIG. 116), and the amino acid sequence thereof is shown in SEQ ID NO: 105 (FIG. 117).

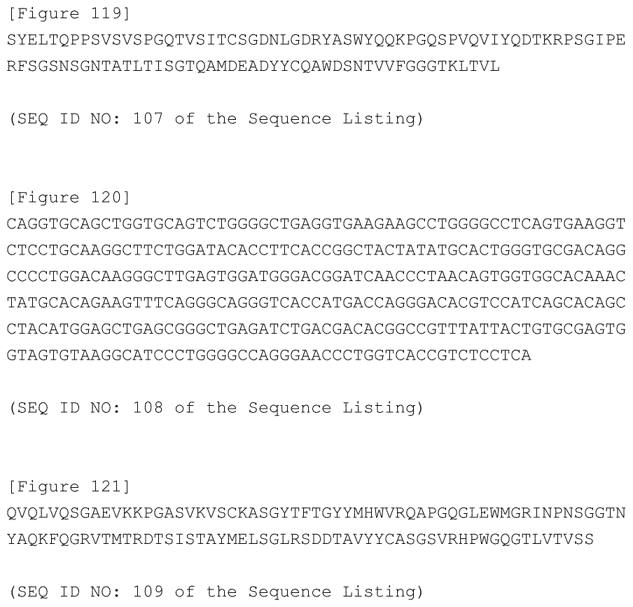

The determined nucleotide sequence of the cDNA encoding the light chain variable region of C3015 is shown in SEQ ID NO: 106 (FIG. 118), and the amino acid sequence thereof is shown in SEQ ID NO: 107 (FIG. 119).

The determined nucleotide sequence of the cDNA encoding the heavy chain variable region of C3022 is shown in SEQ ID NO: 108 (FIG. 120), and the amino acid sequence thereof is shown in SEQ ID NO: 109 (FIG. 121).

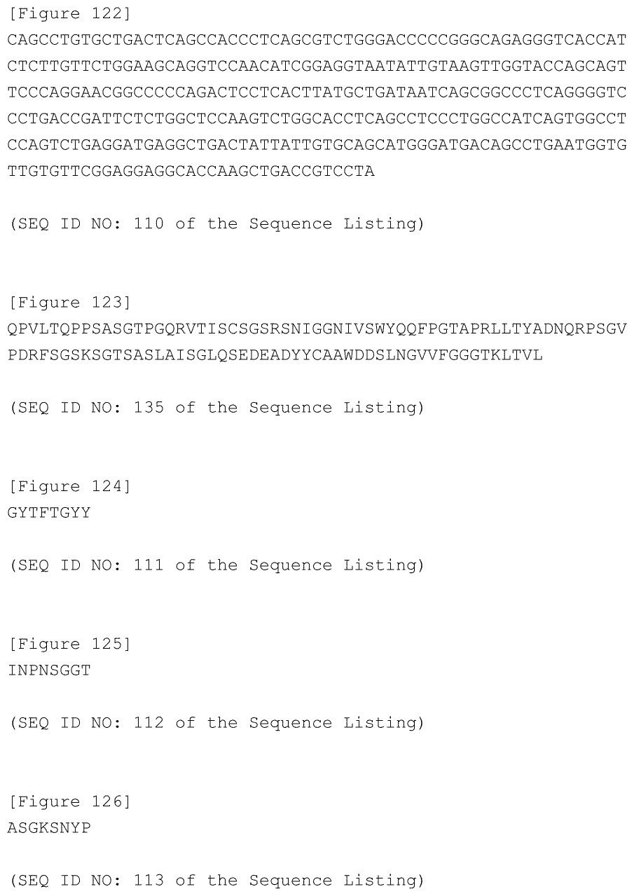

The determined nucleotide sequence of the cDNA encoding the light chain variable region of C3022 is shown in SEQ ID NO: 110 (FIG. 122), and the amino acid sequence thereof is shown in SEQ ID NO: 135 (FIG. 123).

10)-4 Expression and Purification of scFv

The C2037 antibody, C3048 antibody, C3015 antibody, or C3022 antibody scFv was inserted to an expression vector for animal cells such as pcDNA3.1 (Thermo Fisher Scientific Inc.) to construct an scFv expression vector for animal cells. The scFv expression vector for animal cells was transfected to Expi293F cells (Thermo Fisher Scientific Inc.), for transient expression. If necessary, the scFv was purified from the culture supernatant using a His Trap column (GE Healthcare Bio-Sciences Corp.) and a gel filtration column (Superdex 200 Increase, GE Healthcare Bio-Sciences Corp.). The buffer solution containing the scFv dissolved therein was replaced with PBS, and the resulting solution was subjected to the following step “10)-6”.

### 10)-5 Conversion to Full-Length IgG and Expression and Purification of IgG

A full-length IgG form containing C2037, C3048, C3015, or C3022 was prepared by the following method.

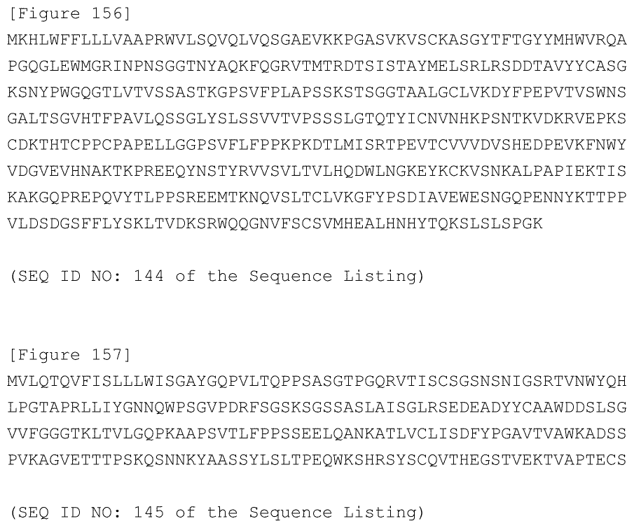

The nucleotide sequences encoding the heavy and light chain variable regions of each antibody identified in Example 10)-3 were linked to a nucleotide sequence encoding a human IgG₁ heavy chain constant region (CH1+Fc region: amino acid sequence positions 135 to 464 of the amino acid sequence represented by SEQ ID NO: 144 (FIG. 156) of the Sequence Listing) and a nucleotide sequence encoding a human IgG₁ light chain constant region (CL: amino acid sequence positions 131 to 236 of the amino acid sequence represented by SEQ ID NO: 145 (FIG. 157) of the Sequence Listing), respectively, by a routine method. The constructs were inserted to an expression vector for animal cells such as pcDNA3.1 (Thermo Fisher Scientific Inc.) to construct an IgG expression vector for animal cells.

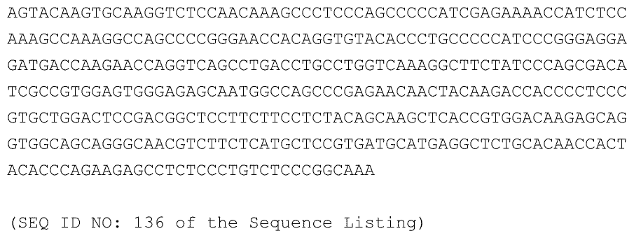

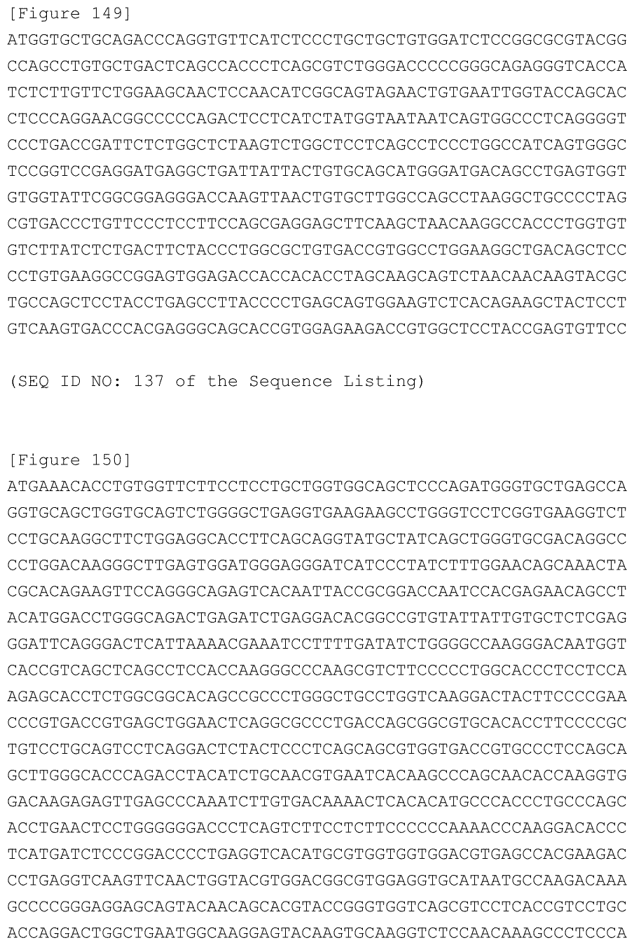

The nucleotide sequence of the constructed IgG expression vector was re-analyzed. It was confirmed that the nucleotide sequence of the full-length heavy chain of the C2037 antibody was the nucleotide sequence represented by SEQ ID NO: 136 (FIG. 148) of the Sequence Listing, and the nucleotide sequence of the full-length light chain was the nucleotide sequence represented by SEQ ID NO: 137 (FIG. 149) of the Sequence Listing.

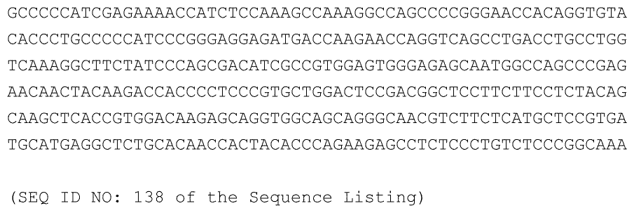

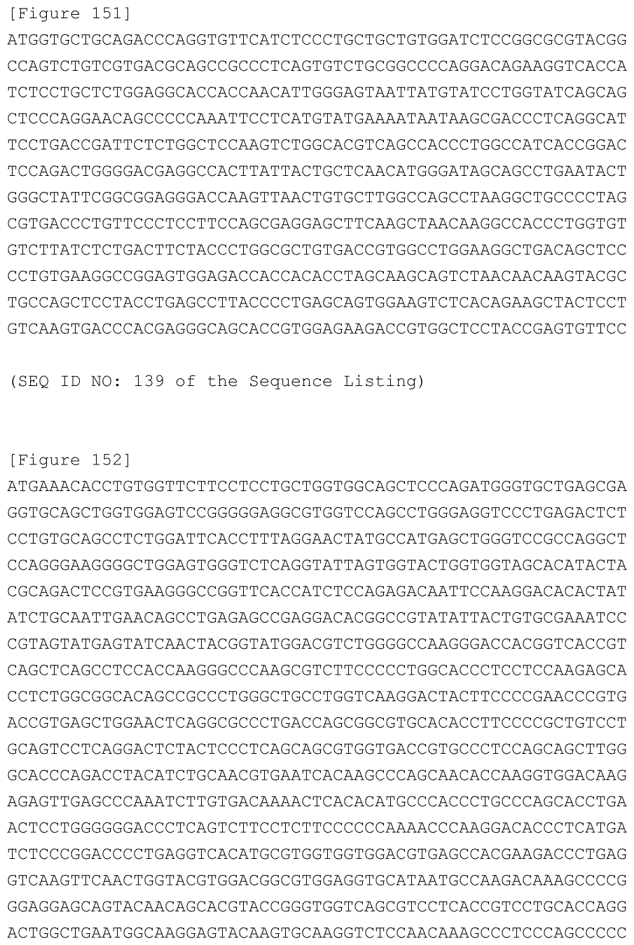

It was confirmed that the nucleotide sequence of the full-length heavy chain of the C3048 antibody was the nucleotide sequence represented by SEQ ID NO: 138 (FIG. 150) of the Sequence Listing, and the nucleotide sequence of the full-length light chain was the nucleotide sequence represented by SEQ ID NO: 139 (FIG. 151) of the Sequence Listing.

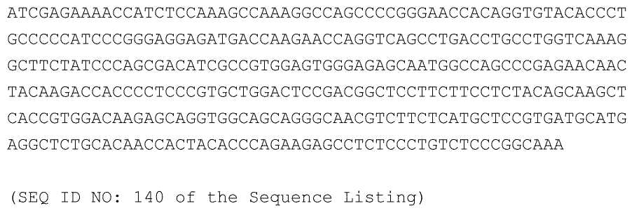

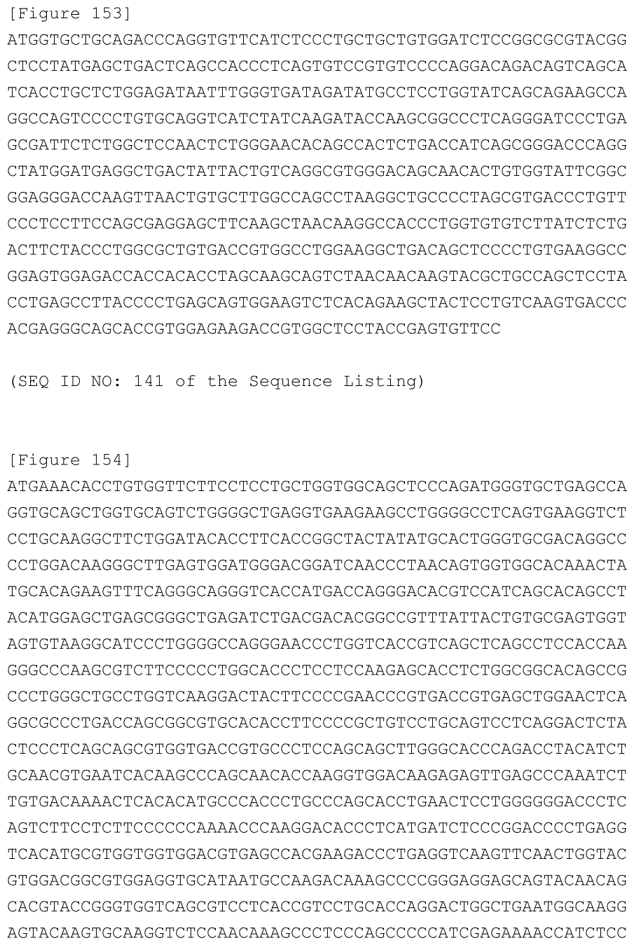

It was confirmed that the nucleotide sequence of the full-length heavy chain of the C3015 antibody was the nucleotide sequence represented by SEQ ID NO: 140 (FIG. 152) of the Sequence Listing, and the nucleotide sequence of the full-length light chain was the nucleotide sequence represented by SEQ ID NO: 141 (FIG. 153) of the Sequence Listing.

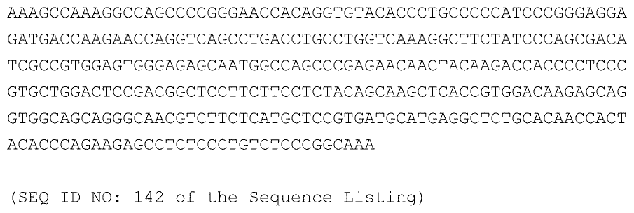

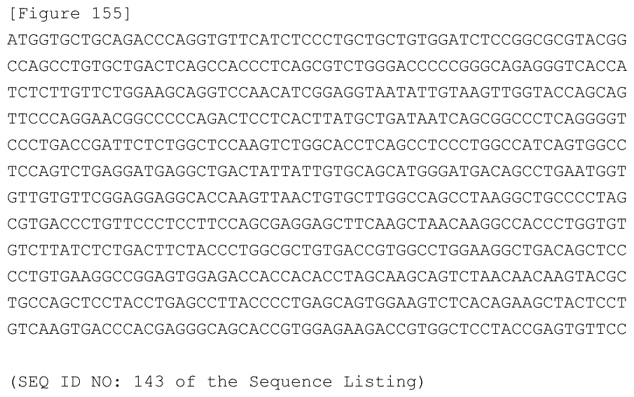

It was confirmed that the nucleotide sequence of the full-length heavy chain of the C3022 antibody was the nucleotide sequence represented by SEQ ID NO: 142 (FIG. 154) of the Sequence Listing, and the nucleotide sequence of the full-length light chain was the nucleotide sequence represented by SEQ ID NO: 143 (FIG. 155) of the Sequence Listing.

The amino acid sequences of the full-length heavy and light chains of the C2037, C3048, C3015, and C3022 antibodies encoded by these sequences were determined from the nucleotide sequences.

The amino acid sequence of the heavy chain of the C2037 antibody was the amino acid sequence represented by SEQ ID NO: 144 (FIG. 156) of the Sequence Listing, and the amino acid sequence of the light chain was the amino acid sequence represented by SEQ ID NO: 145 (FIG. 157) of the Sequence Listing.

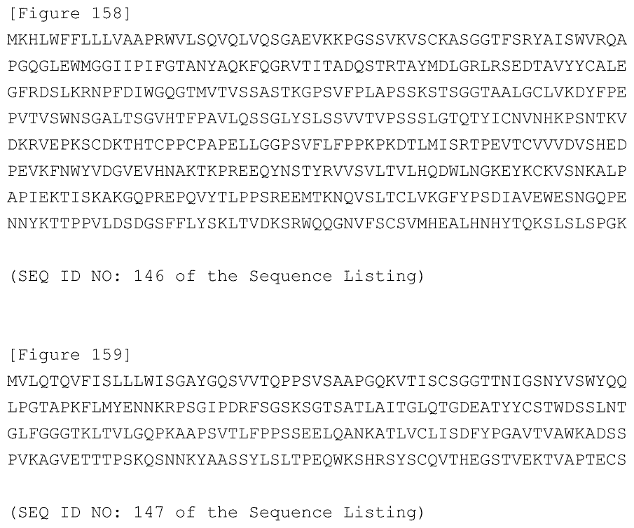

The amino acid sequence of the heavy chain of the C3048 antibody was the amino acid sequence represented by SEQ ID NO: 146 (FIG. 158) of the Sequence Listing, and the amino acid sequence of the light chain was the amino acid sequence represented by SEQ ID NO: 147 (FIG. 159) of the Sequence Listing.

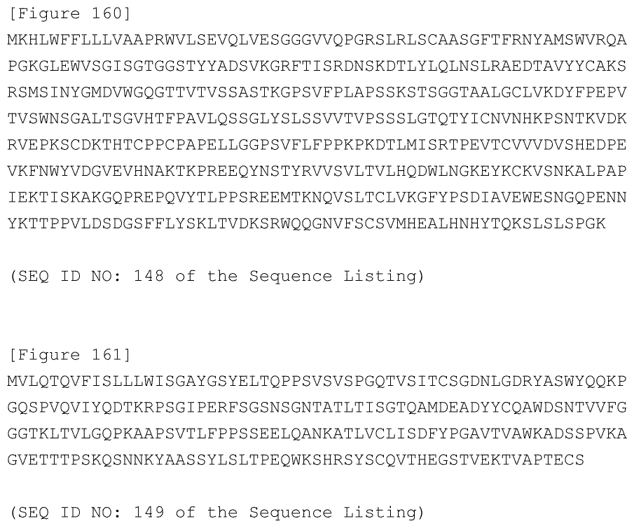

The amino acid sequence of the heavy chain of the C3015 antibody was the amino acid sequence represented by SEQ ID NO: 148 (FIG. 160) of the Sequence Listing, and the amino acid sequence of the light chain was the amino acid sequence represented by SEQ ID NO: 149 (FIG. 161) of the Sequence Listing.

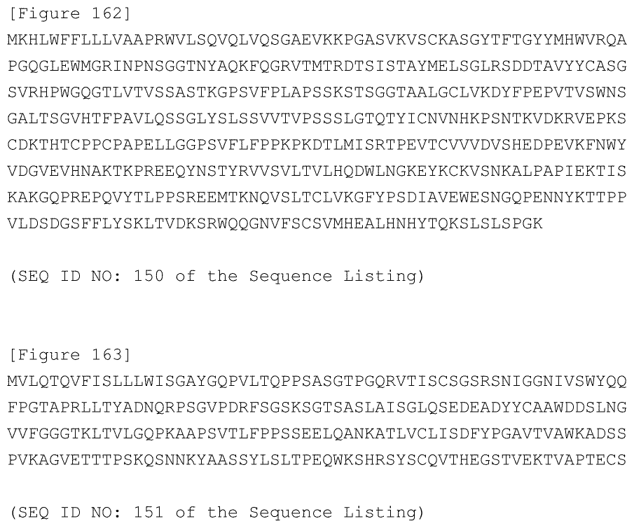

The amino acid sequence of the heavy chain of the C3022 antibody was the amino acid sequence represented by SEQ ID NO: 150 (FIG. 162) of the Sequence Listing, and the amino acid sequence of the light chain was the amino acid sequence represented by SEQ ID NO: 151 (FIG. 163) of the Sequence Listing.

The IgG form of the C2037, C3048, C3015, or C3022 antibody was transiently expressed by the transfection of FreeStyle 293F cells (Thermo Fisher Scientific Inc.) with the IgG expression vector for animal cells. If necessary, the IgG form was purified using a protein A affinity column (HiTrap Mab Select SuRe, GE Healthcare Bio-Sciences Corp.). Then, the buffer solution containing the IgG dissolved therein was replaced with PBS using Vivaspin 20 (7k MWCO, GE Healthcare Bio-Sciences Corp.), and the resulting solution was subjected to the following steps 10)-7 and 10)-9.

10)-6 Confirmation of Binding of scFv to GPRC5D by ELISA

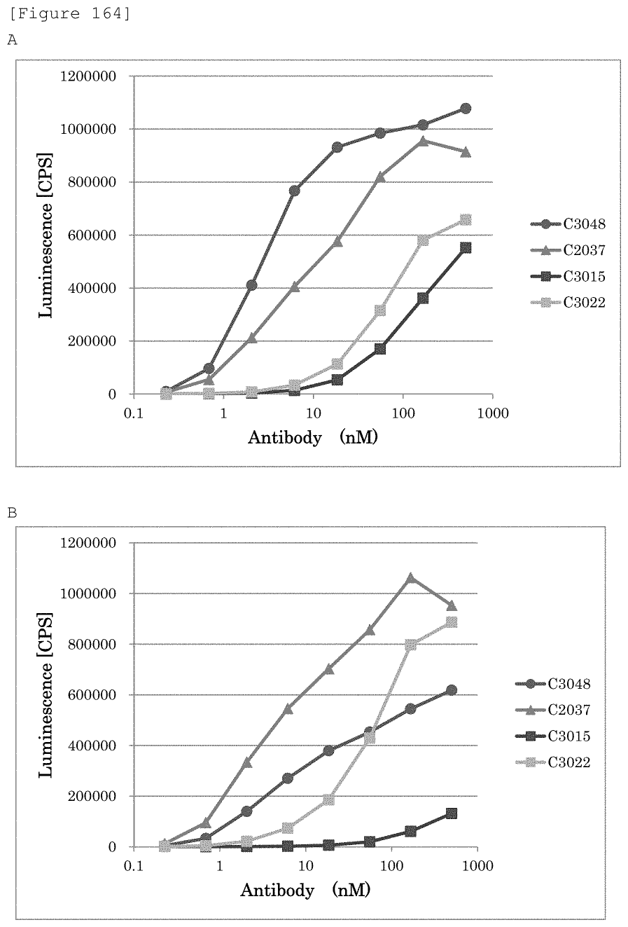

NeutrAvidin diluted to 1 μg/mL with PBS was added in an amount of 50 μL/well to 96-well Maxi-sorp plate (Black, Nunc/Thermo Fisher Scientific Inc.), and the plate was left standing overnight at 4° C. for immobilization. After washing three times with an ELISA buffer, the amino-terminal peptide of the biotinylated human or cynomolgus monkey GPRC5D (used in Example 10)-1) diluted to 1 μg/mL with PBS was added thereto, and the plate was shaken at room temperature for 1 hour. After washing three times with an ELISA buffer, the plate was blocked with Blocker Casein and washed three times with an ELISA buffer. Then, the C2037, C3048, C3015, or C3022 scFv was added to the plate and reacted at room temperature for 2 hours. After washing three times with an ELISA buffer, a horseradish peroxidase (HRP)-labeled anti-FLAG antibody diluted 5000-fold with an ELISA buffer was added thereto in an amount of 50 μL/well and reacted at room temperature for 1 hour. After washing five times with an ELISA buffer, SuperSignal Pico ELISA Chemiluminescent substrate was added. After 10 minutes, the chemiluminescence was measured using a plate reader. As a result, the C2037, C3048, C3015, and C3022 scFvs were found to bind to the amino-terminal peptides of human GPRC5D (FIG. 164A) and cynomolgus monkey GPRC5D (FIG. 164B).

### 10)-7 Confirmation of Binding of IgG to GPRC5D by ELISA

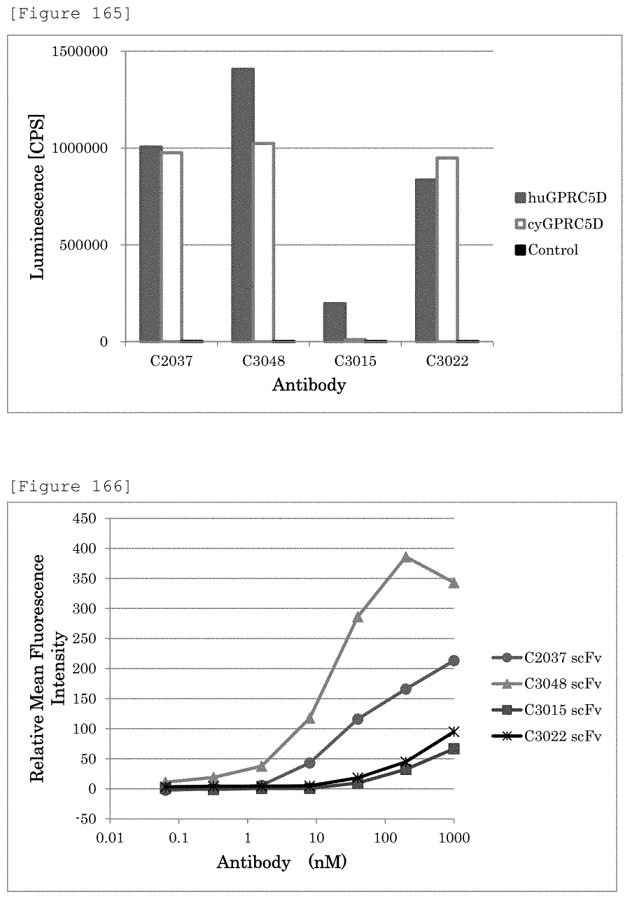

NeutrAvidin diluted to 1 μg/mL with PBS was added in an amount of 50 μL/well to 96-well Maxi-sorp plate, and the plate was left standing overnight at 4° C. for immobilization. After washing three times with an ELISA buffer, the amino-terminal peptide of the biotinylated human or cynomolgus monkey GPRC5D (used in Example 10)-1) diluted to 1 μg/mL with PBS was added thereto, and the plate was shaken at room temperature for 1 hour. After washing three times with an ELISA buffer, the plate was blocked with Blocker Casein and washed three times with an ELISA buffer. Then, the culture solution of the IgG-expressing FreeStyle 293F cells was added to the plate and reacted at room temperature for 2 hours. After washing three times with an ELISA buffer, a horseradish peroxidase (HRP)-labeled anti-human Fab antibody (Jackson ImmunoResearch Laboratories, Inc.) diluted 2500-fold with ELISA buffer was added thereto in an amount of 50 μL/well and reacted at room temperature for 1 hour. After washing five times with an ELISA buffer, SuperSignal Pico ELISA Chemiluminescent substrate was added. After 10 minutes, the chemiluminescence was measured using a plate reader. As a result, the C2037, C3048, C3015 and C3022 IgG forms were found to bind to the amino-terminal peptides of human GPRC5D and cynomolgus monkey GPRC5D (FIG. 165).

10)-8 Binding of scFv to Endogenous Human GPRC5D-Expressing Cell (KMS-34)

The KMS-34 cells were recovered by centrifugation, washed twice with a FACS buffer (PBS containing 0.5% BSA and 2 mM EDTA, pH 7.4), and then suspended in the same solution as above. The C2037, C3048, C3015 or C3022 scFv was added to the obtained cell suspension, and the mixture was left standing at 4° C. for 2 hours. After washing twice with a FACS buffer, the cells were suspended by the addition of an anti-FLAG antibody (Sigma-Aldrich Corp.), and the mixture was further left standing at 4° C. for 1 hour. After washing twice with a FACS buffer, the cells were suspended by the addition of Alexa 488-labeled anti-mouse IgG antibody (Jackson ImmunoResearch Laboratories, Inc.), and the mixture was further left standing at 4° C. for 1 hour. After washing twice with a FACS buffer, the cells were fixed in 1% PFA (prepared from a 32% paraformaldehyde solution (Electron Microscopy Sciences)), followed by detection using a flow cytometer (FACSCanto™ II; Becton, Dickinson and Company). The data was analyzed using Flowjo (Tree Star, Inc.).

As a result, the C2037, C3048, C3015, and C3022 scFvs were found to bind to human GPRC5D-expressing cells (FIG. 166).

### 10)-9 Binding of IgG to Endogenous Human GPRC5D-Expressing Cell (KHM-1B)

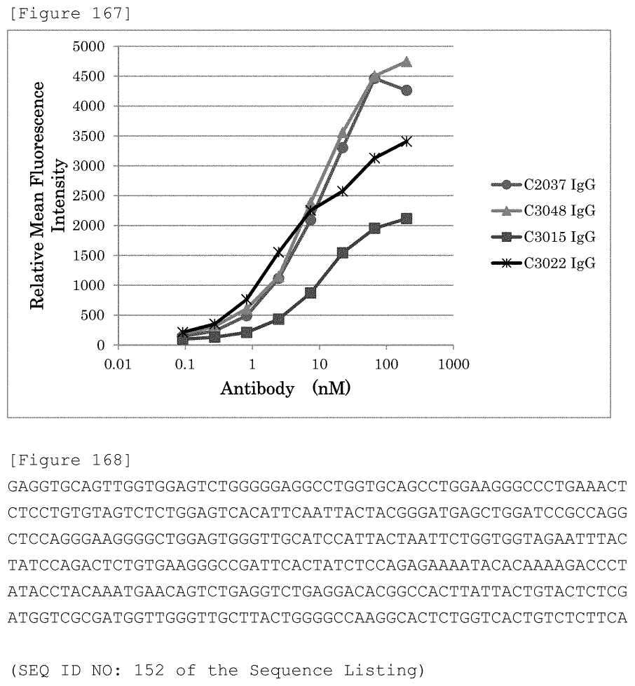

Human multiple myeloma cell line KHM-1B cells expressing GPRC5D were adjusted to a concentration of 2×10⁶ cells/mL with PBS containing 5% FBS, inoculated in an amount of 100 μL/well to a 96-well U-bottomed microplate, and centrifuged to remove the supernatant. The culture supernatant of each human anti-GPRC5D antibody (full-length IgG form containing C2037, C3048, C3015, or C3022) obtained in Example 10)-5 or Human IgG isotype control antibody (Calbiochem/Merck Millipore Corp.) adjusted to 14 ng/mL to 30 μg/mL was added thereto in an amount of 100 μL/well, and the plate was left standing at 4° C. for 1 hour. The cells were washed twice with PBS containing 5% FBS. Then, R-Phycoerythrin AffiniPure F(ab′)2 Fragment Goat Anti-Human IgG, Fcγ Fragment Specific (Jackson ImmunoResearch Laboratories, Inc.) diluted 100-fold with PBS containing 5% FBS was added thereto in an amount of 100 μL/well, and the plate was left standing at 4° C. for 1 hour. The cells were washed twice with PBS containing 5% FBS and then resuspended in PBS containing 5% FBS, followed by detection using a flow cytometer (FACSCanto™ II; Becton, Dickinson and Company). The data was analyzed using Flowjo (Tree Star, Inc.). The PE fluorescence intensity of the cell fraction was plotted to a histogram, and the mean fluorescence intensity (MFI) was calculated. The MFI value of the control antibody was subtracted from the MFI value of the GPRC5D antibody to calculate a relative value of MFI (rMFI). As a result, the C2037, C3048, C3015, and C3022 IgG forms were found to bind to human GPRC5D-expressing cells (FIG. 167).

### 10)-10 Preparation and Evaluation of Modified Forms of C3022 and C3048

10)-10-1 Obtainment of Modified Forms

A library was constructed by use of a method for introducing a mutation by PCR using the C3022 and C3048 genes as templates (Zaccolo, et al., J. Mol. Biol. (1996) 255, 589-603) or a method which involves synthesizing oligomers such that as to all residues of CDRs, each residue was mutated to 19 types of amino acids other than wild-type amino acids to construct a library (oligo-based library). The library was screened for clones having the high ability to bind, and their nucleotide sequences were determined. The identified high binding mutations were combined to obtain a high binding mutant E1018 of C3022 and a high binding mutant D1012 of C3048.

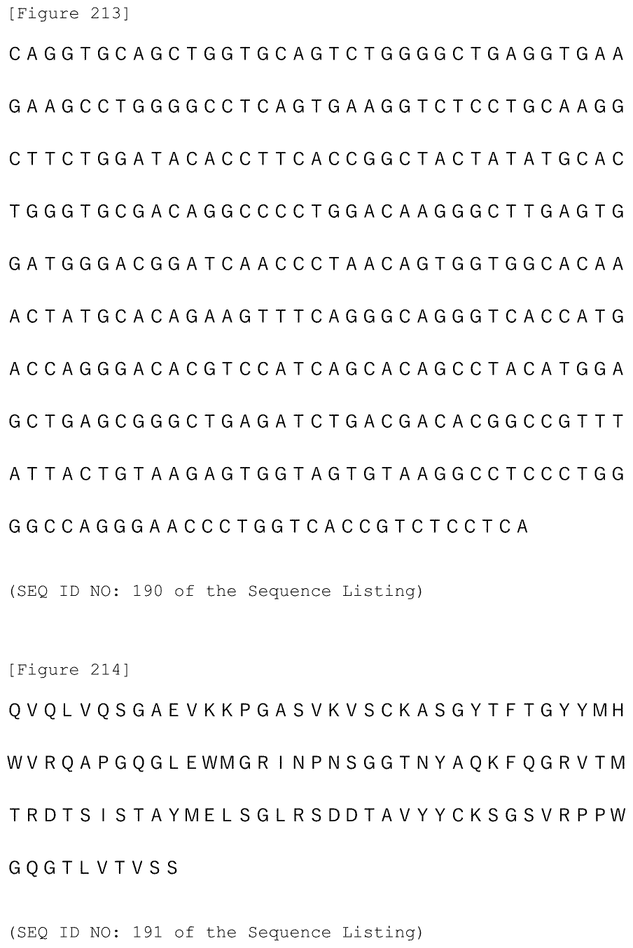

The nucleotide sequence of the cDNA encoding the heavy chain variable region of the obtained E1018 is shown in SEQ ID NO: 190 (FIG. 213), and the amino acid sequence thereof is shown in SEQ ID NO: 191 (FIG. 214).

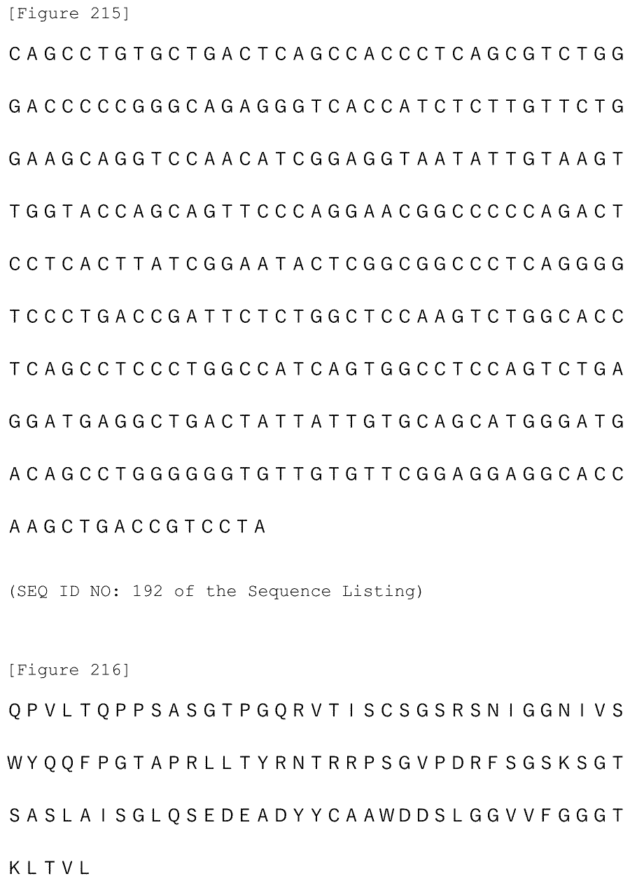

The nucleotide sequence of the cDNA encoding the light chain variable region of the obtained E1018 is shown in SEQ ID NO: 192 (FIG. 215), and the amino acid sequence thereof is shown in SEQ ID NO: 193 (FIG. 216).

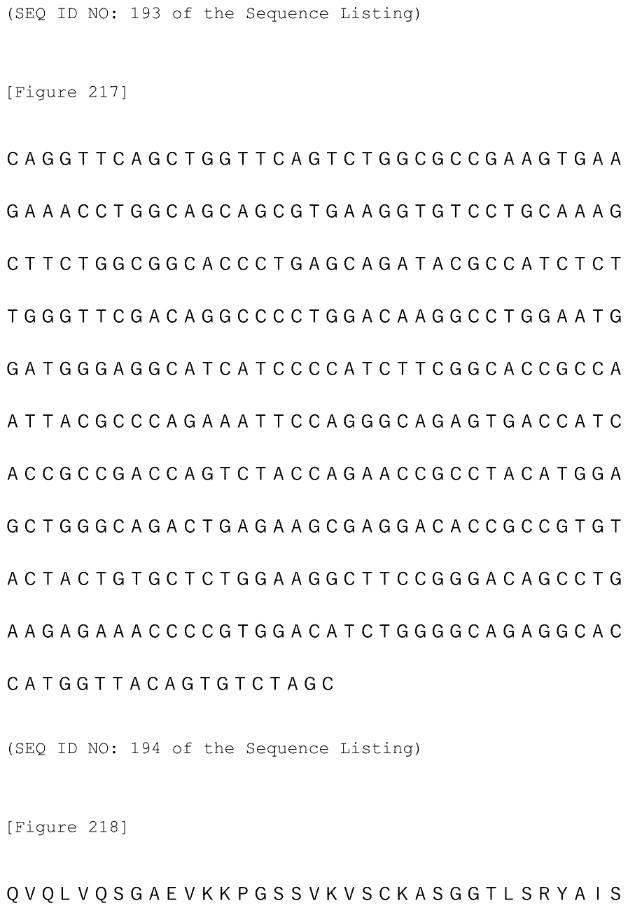

The nucleotide sequence of the cDNA encoding the heavy chain variable region of the obtained D1012 is shown in SEQ ID NO: 194 (FIG. 217), and the amino acid sequence thereof is shown in SEQ ID NO: 195 (FIG. 218).

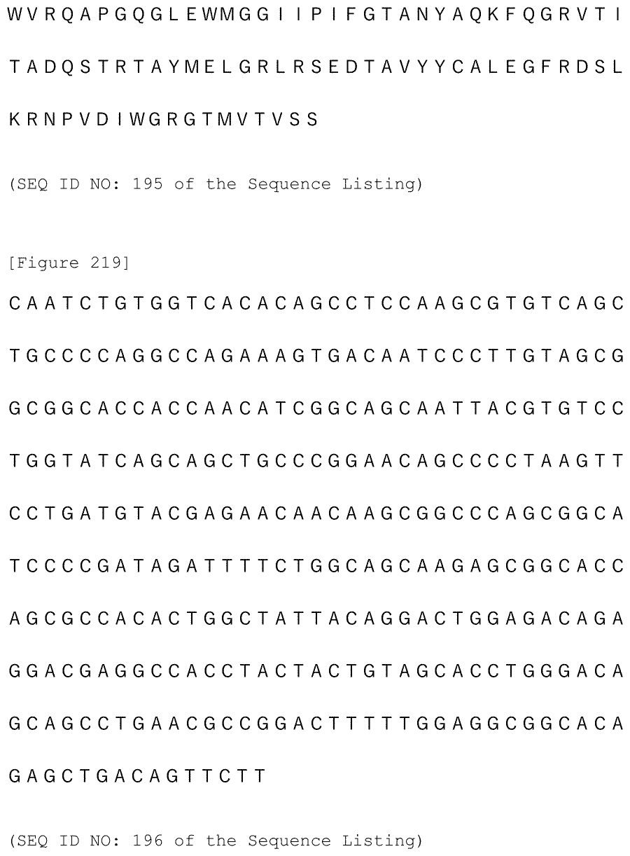

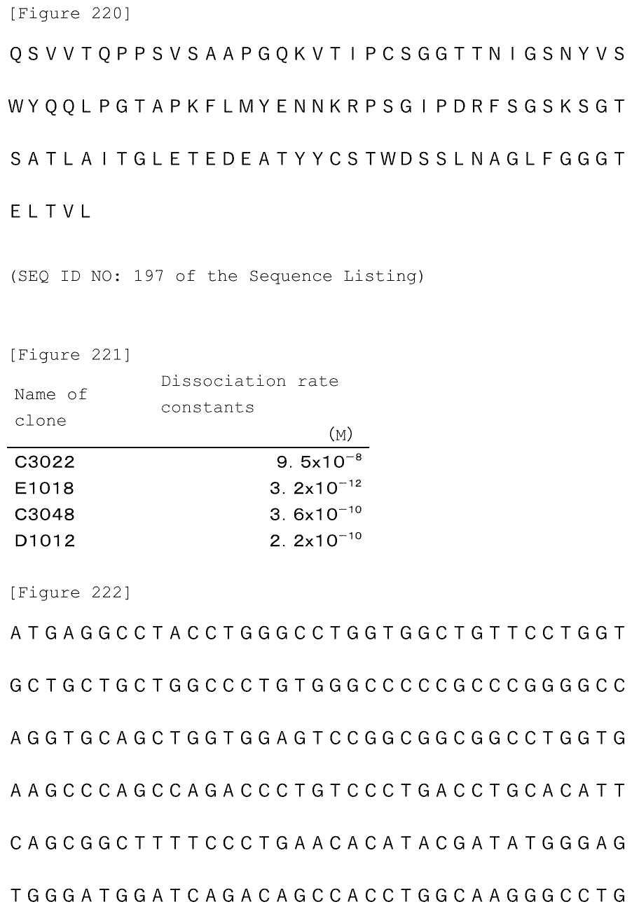

The nucleotide sequence of the cDNA encoding the light chain variable region of the obtained D1012 is shown in SEQ ID NO: 196 (FIG. 219), and the amino acid sequence thereof is shown in SEQ ID NO: 197 (FIG. 220).

### 10)-10-2 Confirmation of Binding to GPRC5D Using Biacore

The binding activity of the anti-GPRC5D antibodies against the amino-terminal peptide of human GPRC5D was tested by SPR using Biacore T200. The amino-terminal peptide of biotinylated human GPRC5D diluted to 2 nM with HBS-EP+ (manufactured by GE Healthcare Bio-Sciences Corp.) was immobilized on Sensor Chip CAP (manufactured by GE Healthcare Bio-Sciences Corp.) by contact at a rate of 10 μL/min for 180 seconds. Then, Kd was calculated by kinetic analysis using a plurality of concentrations of each scFv diluted with HBS-EP+ as analytes. As a result, the E1018 and D1012 scFvs were found to bind to the amino-terminal peptide of human GPRC5D more strongly than C3022 and C3048, respectively (FIG. 221).

### Example 11

### Construction of Anti-CD3 Antibody Expression Vector

11)-1 Construction of Rat Anti-CD3 scFv Antibody Expression Vector

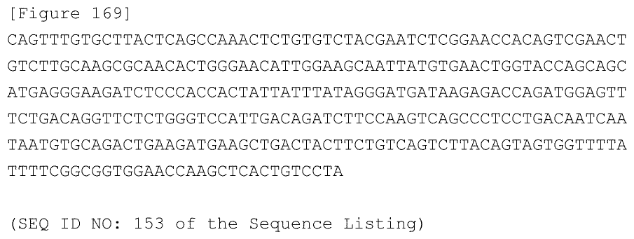

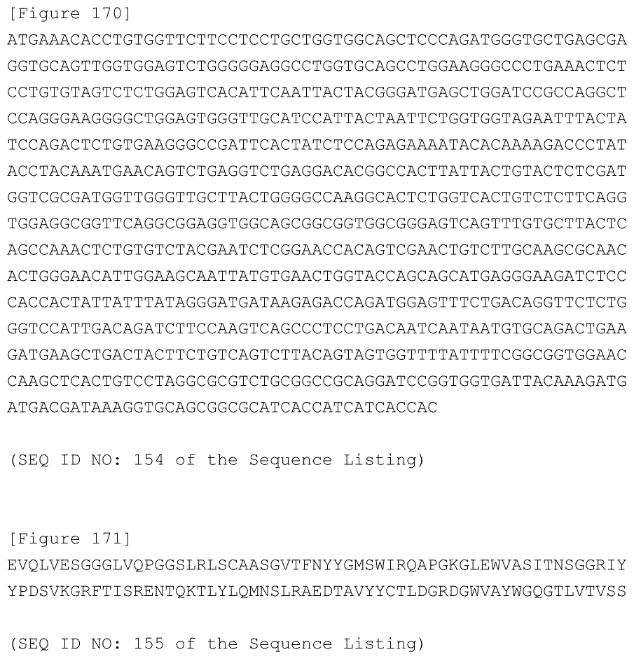

A rat anti-CD3 monoclonal antibody-producing hybridoma was prepared from lymph node or spleen of a rat immunized by the DNA immunization method. cDNAs encoding VH and VL of the monoclonal antibody was sequenced from the hybridoma, and a single chain Fv expression vector was prepared. Specifically, the VH DNA fragment of SEQ ID NO: 152 (FIG. 168) amplified by PCR, the DNA fragment of the linker to be inserted between VH and VL, and a DNA fragment amplified by PCR in which a DNA sequence encoding a FLAG-His tag was added to a region containing the VL DNA sequence of SEQ ID NO: 153 (FIG. 169) such that the FLAG-His tag was located at the carboxyl terminus, were fused using In-Fusion HD cloning kit (Clontech Laboratories, Inc.) to prepare a single chain Fv expression vector pC3E-7000 containing the nucleotide sequence of SEQ ID NO: 154 (FIG. 170) in ORF.

11)-2 Construction of Humanized Anti-CD3 scFv Antibody Expression Vector pC3E-7034

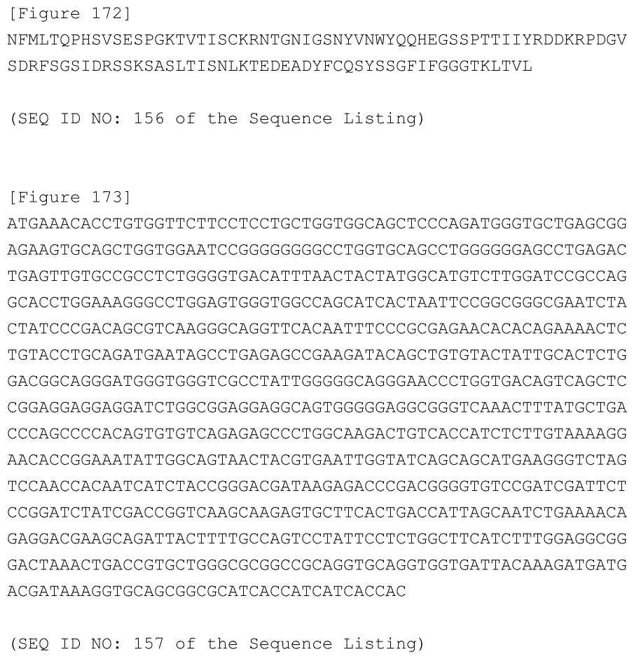

A DNA fragment comprising a DNA sequence of scFv containing a light chain variable region (SEQ ID NO: 156 (FIG. 172))-containing region connected to the carboxyl terminus of SEQ ID NO: 155 (FIG. 171) via a 15-amino acid flexible linker, and 15-base additional sequences upstream and downstream thereof was synthesized (GeneArt Artificial Gene Synthesis Service). A region containing the C3E-7034 DNA and its upstream and downstream additional sequences was amplified by PCR using this DNA fragment as a template to obtain an insert DNA fragment. A vector region except for an scFv region was amplified by PCR using the expression vector pC3E-7000 prepared in Example 11)-1 as a template to obtain a vector fragment. These DNA fragments were fused using In-Fusion HD cloning kit (Clontech Laboratories, Inc.) to prepare an expression vector containing the nucleotide sequence of SEQ ID NO: 157 (FIG. 173) in ORF. The obtained expression vector was designated as “pC3E-7034”.

11)-3 Construction of Humanized Anti-CD3 scFv Antibody Expression Vector pC3E-7035

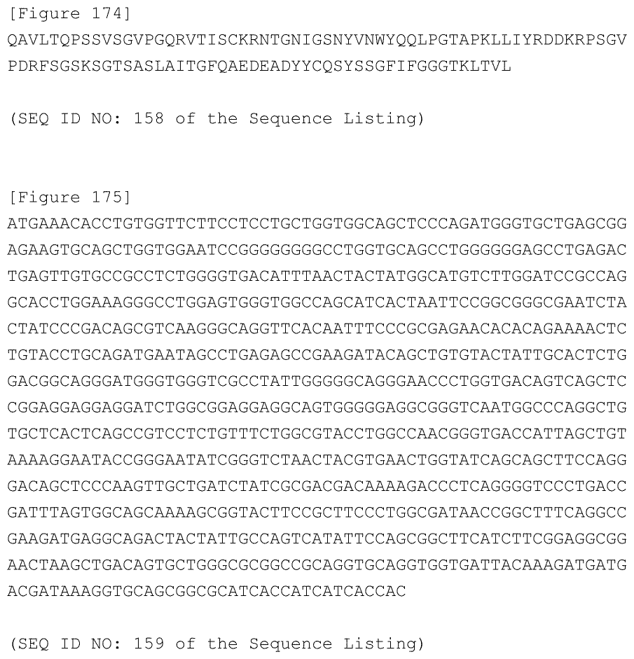

A DNA fragment comprising a DNA sequence of scFv containing a light chain variable region of SEQ ID NO: 158 (FIG. 174) connected to the carboxyl terminus of SEQ ID NO: 155 (FIG. 171) via a 17-amino acid flexible linker, and 15-base additional sequences upstream and downstream thereof was synthesized (GeneArt Artificial Gene Synthesis Service). An expression vector containing the nucleotide sequence of SEQ ID NO: 159 (FIG. 175) in ORF was constructed in the same way as in Example 11)-2. The obtained expression vector was designated as “pC3E-7035”.

11)-4 Construction of Humanized Anti-CD3 scFv Antibody Expression Vector pC3E-7036

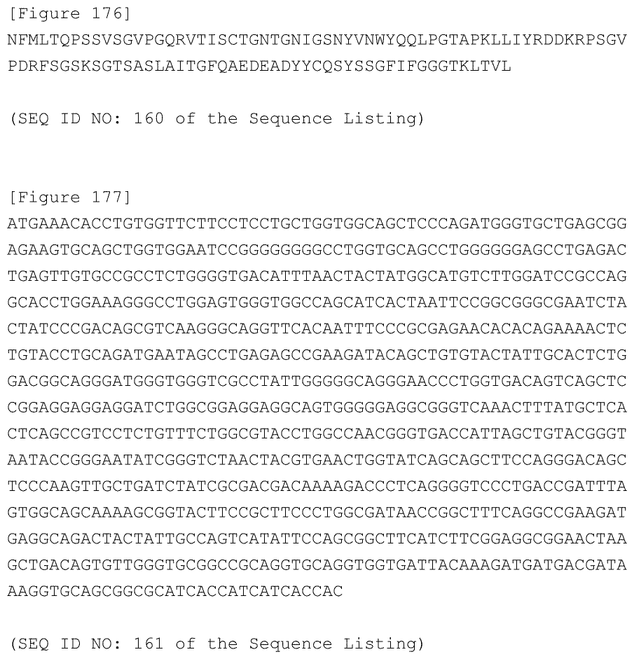

A DNA fragment comprising a DNA sequence of scFv containing a light chain variable region of SEQ ID NO: 160 (FIG. 176) connected to the carboxyl terminus of SEQ ID NO: 155 (FIG. 171) via a 15-amino acid flexible linker, and 15-base additional sequences upstream and downstream thereof was synthesized (GeneArt Artificial Gene Synthesis Service). A C3E-7036 expression vector containing the nucleotide sequence of SEQ ID NO: 161 (FIG. 177) in ORF was constructed in the same way as in Example 11)-2. The obtained expression vector was designated as “pC3E-7036”.

### Example 12. Preparation of Anti-GPRC5D-Anti-CD3 Bispecific Molecule

12)-1 Preparation of anti-GPRC5D-Anti-CD3 Bispecific Molecule Expression Vector

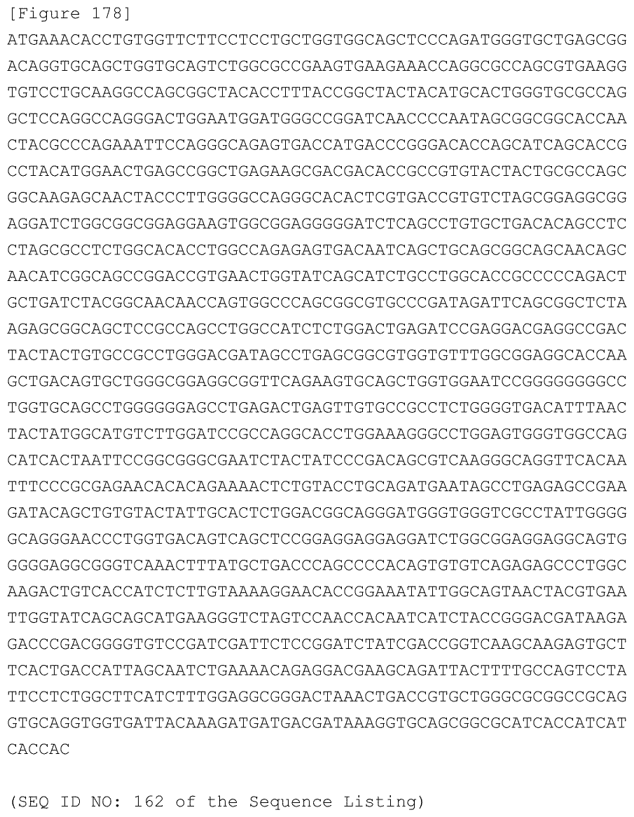

A DNA sequence of a region containing the C2037 antibody scFv, a portion of a human antibody heavy chain signal sequence, and an added linker to link scFvs was amplified by PCR using the DNA sequence of the C2037 antibody scFv prepared in Example 10)-4 as a template to obtain an insert DNA. Also, the whole vector region containing the anti-CD3 scFv DNA was amplified by PCR using the expression vector pC3E-7034 prepared in Example 11)-2 as a template and primers encoding a signal sequence and the amino-terminal sequence of the anti-CD3 scFv antibody to obtain a vector fragment. These DNA fragments were fused using In-Fusion HD cloning kit (Clontech Laboratories, Inc.) to construct an anti-GPRC5D-anti-CD3 bispecific molecule expression vector containing the nucleotide sequence of SEQ ID NO: 162 (FIG. 178) in ORF. The obtained expression vector was designated as “pC2037-C3E7034”.

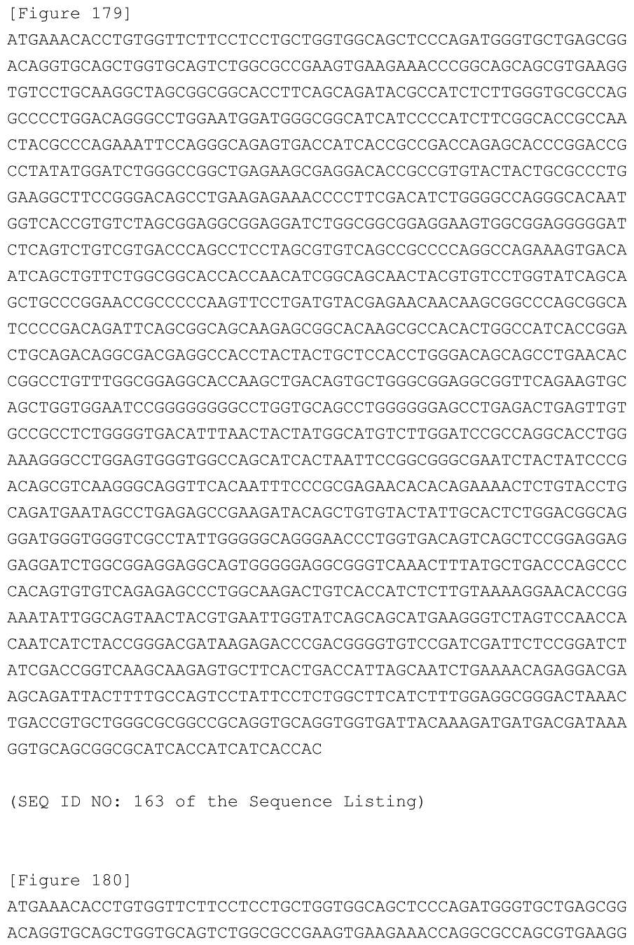

An anti-GPRC5D-anti-CD3 bispecific molecule expression vector containing the nucleotide sequence of SEQ ID NO: 163 (FIG. 179) in ORF was constructed in the same way as above using the DNA sequence of the C3048 antibody scFv and pC3E-7034 as templates. The obtained expression vector was designated as “pC3048-C3E7034”.

An anti-GPRC5D-anti-CD3 bispecific molecule expression vector containing the nucleotide sequence of SEQ ID NO: 164 (FIG. 180) in ORF was constructed in the same way as above using the DNA sequence of the C3022 antibody scFv and pC3E-7034 as templates. The obtained expression vector was designated as “pC3022-C3E7034”.

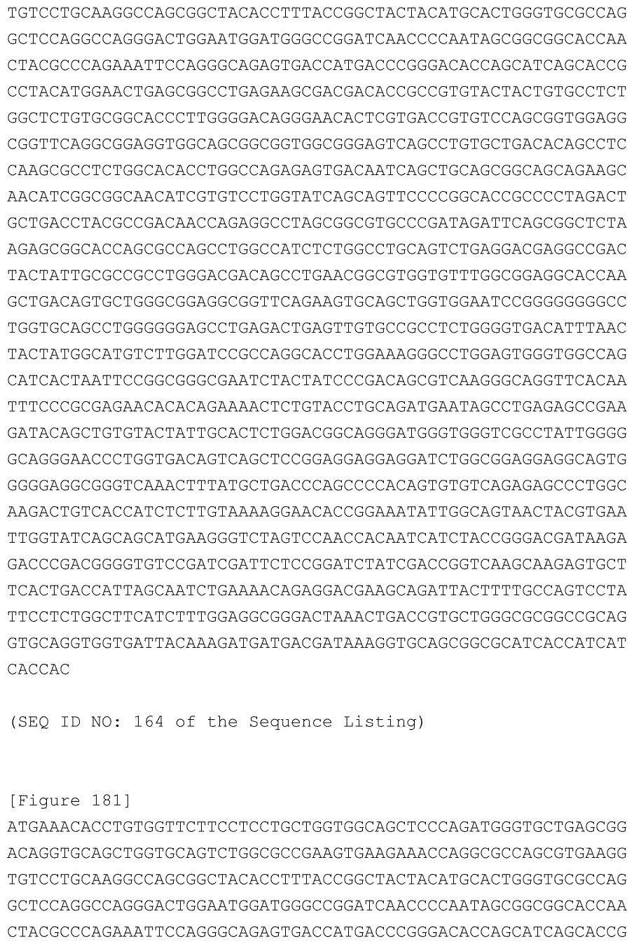

An anti-GPRC5D-anti-CD3 bispecific molecule expression vector containing the nucleotide sequence of SEQ ID NO: 165 (FIG. 181) in ORF was constructed in the same way as above using the DNA sequence of the C2037 antibody scFv and pC3E-7035 as templates. The obtained expression vector was designated as “pC2037-C3E7035”.

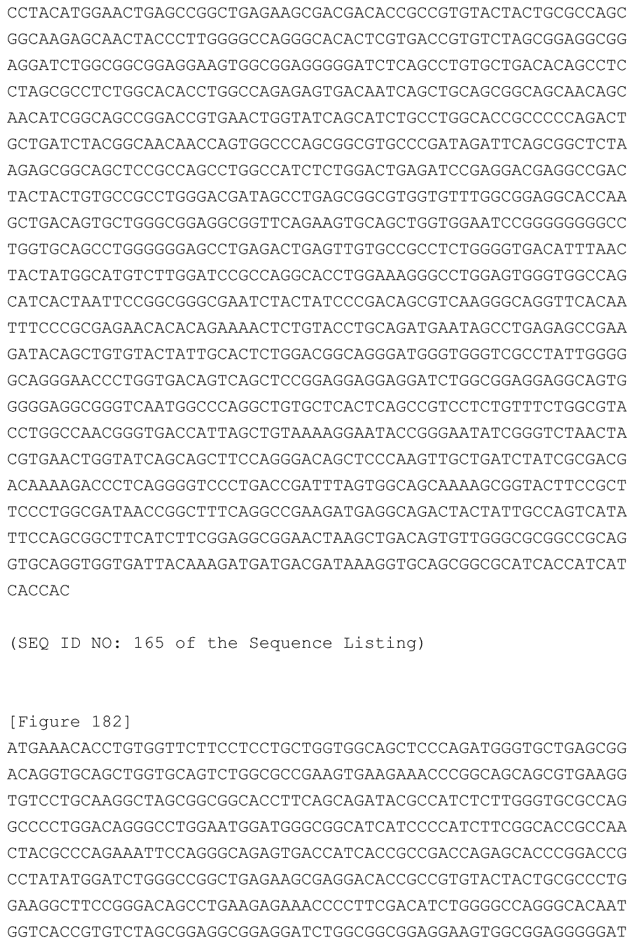

An anti-GPRC5D-anti-CD3 bispecific molecule expression vector containing the nucleotide sequence of SEQ ID NO: 166 (FIG. 182) in ORF was constructed in the same way as above using the DNA sequence of the C3048 antibody scFv and pC3E-7035 as templates. The obtained expression vector was designated as “pC3048-C3E7035”.

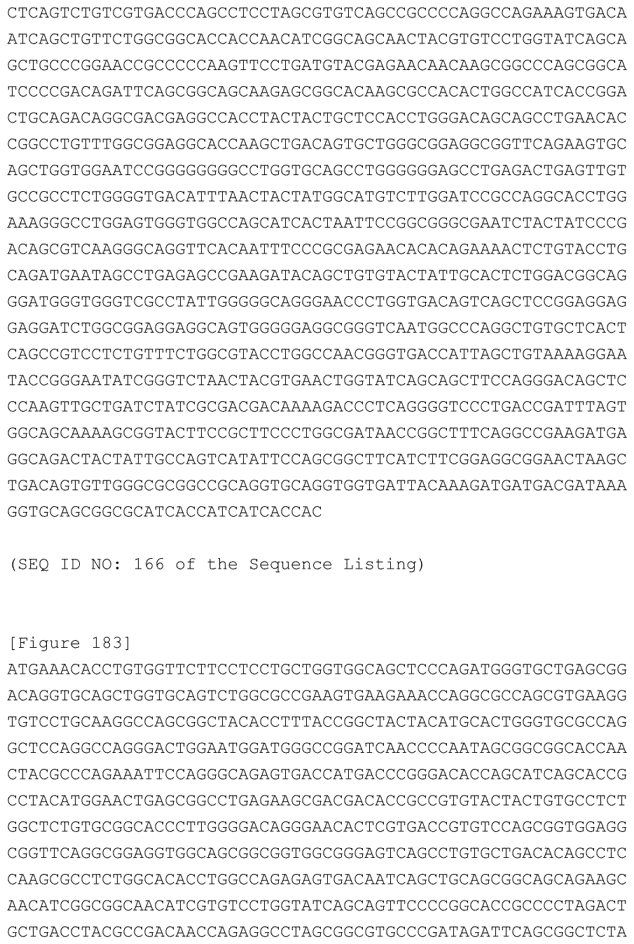

An anti-GPRC5D-anti-CD3 bispecific molecule expression vector containing the nucleotide sequence of SEQ ID NO: 167 (FIG. 183) in ORF was constructed in the same way as above using the DNA sequence of the C3022 antibody scFv and pC3E-7035 as templates. The obtained expression vector was designated as “pC3022-C3E7035”.

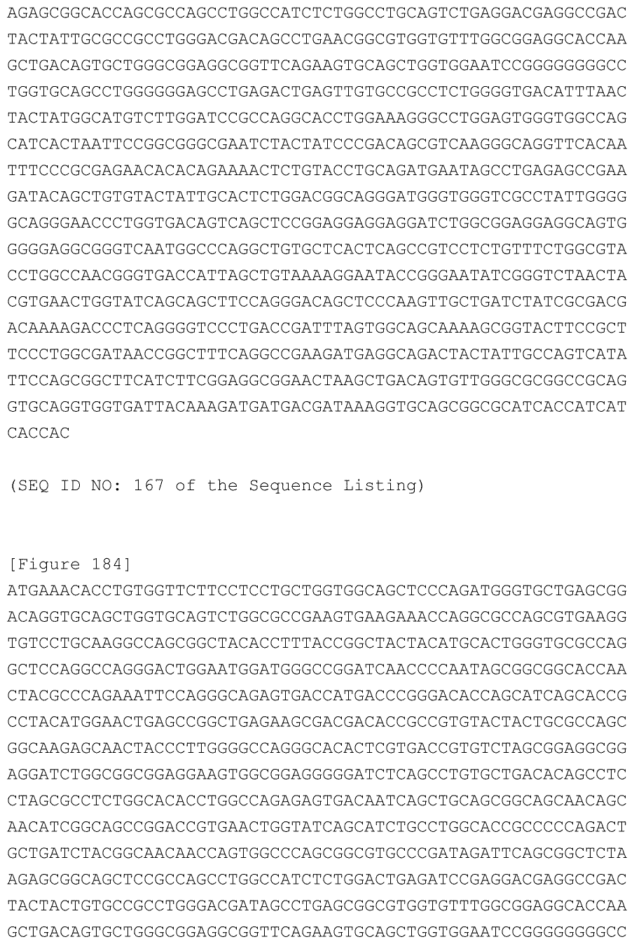

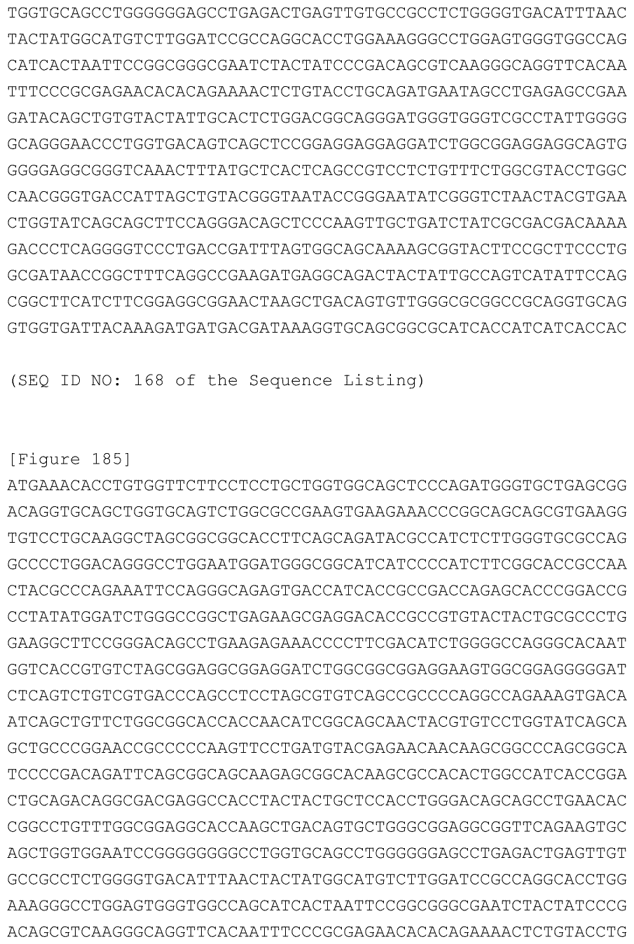

An anti-GPRC5D-anti-CD3 bispecific molecule expression vector containing the nucleotide sequence of SEQ ID NO: 168 (FIG. 184) in ORF was constructed in the same way as above using the DNA sequence of the C2037 antibody scFv and pC3E-7036 as templates. The obtained expression vector was designated as “pC2037-C3E7036”.

An anti-GPRC5D-anti-CD3 bispecific molecule expression vector containing the nucleotide sequence of SEQ ID NO: 169 (FIG. 185) in ORF was constructed in the same way as above using the DNA sequence of the C3048 antibody scFv and pC3E-7036 as templates. The obtained expression vector was designated as “pC3048-C3E7036”.

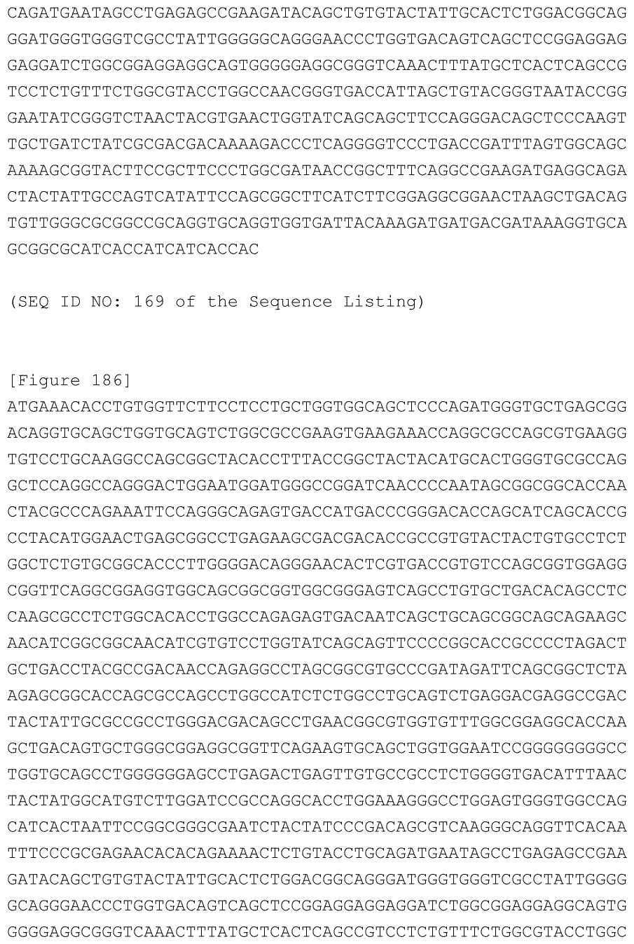

An anti-GPRC5D-anti-CD3 bispecific molecule expression vector containing the nucleotide sequence of SEQ ID NO: 170 (FIG. 186) in ORF was constructed in the same way as above using the DNA sequence of the C3022 antibody scFv and pC3E-7036 as templates. The obtained expression vector was designated as “pC3022-C3E7036”.

### 12)-2 Expression and Purification of Anti-GPRC5D-Anti-CD3 Bispecific Molecule

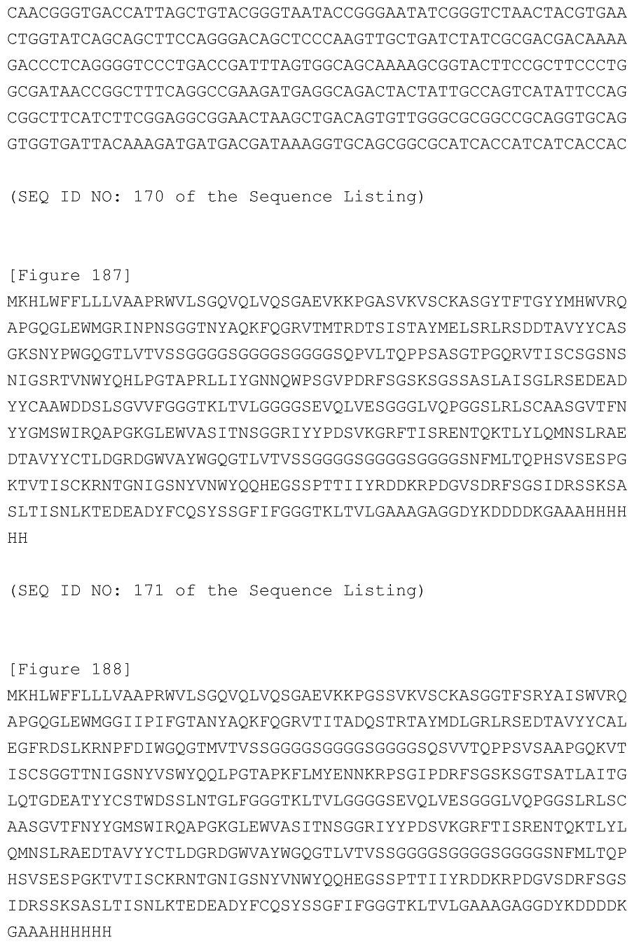

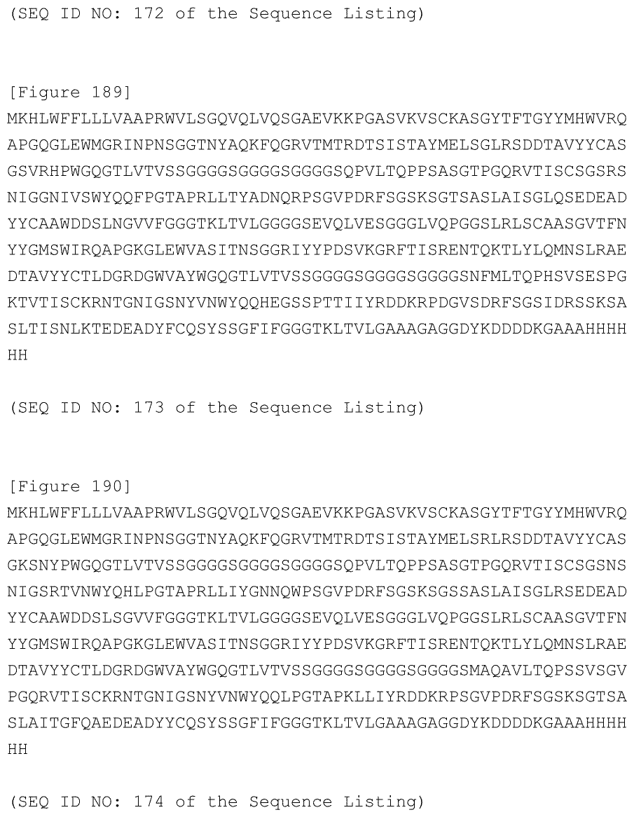

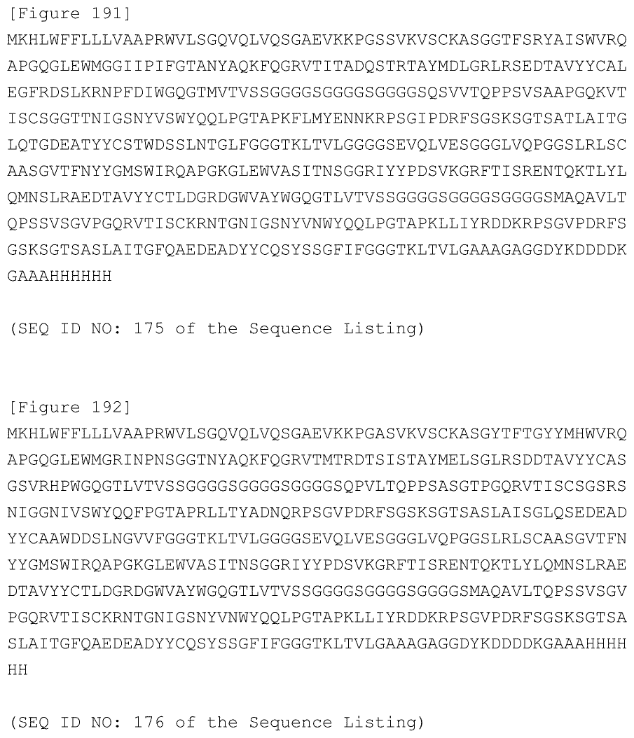

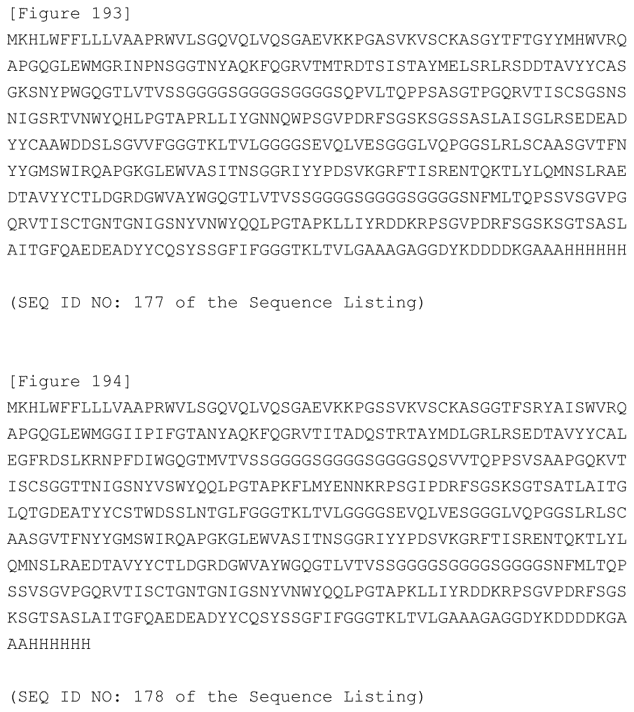

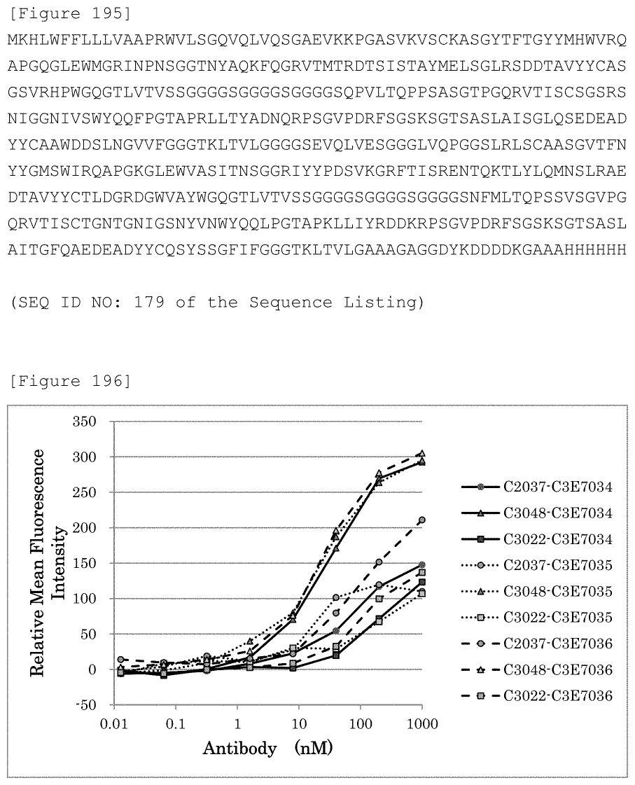

C2037-C3E7034 to C3022-C3E7036 were expressed and purified in the same way as in Example 10)-4. The amino acid sequence of C2037-C3E7034 is described in SEQ ID NO: 171 (FIG. 187). The amino acid sequence of C3048-C3E7034 is described in SEQ ID NO: 172 (FIG. 188). The amino acid sequence of C3022-C3E7034 is described in SEQ ID NO: 173 (FIG. 189). The amino acid sequence of C2037-C3E7035 is described in SEQ ID NO: 174 (FIG. 190). The amino acid sequence of C3048-C3E7035 is described in SEQ ID NO: 175 (FIG. 191). The amino acid sequence of C3022-C3E7035 is described in SEQ ID NO: 176 (FIG. 192). The amino acid sequence of C2037-C3E7036 is described in SEQ ID NO: 177 (FIG. 193). The amino acid sequence of C3048-C3E7036 is described in SEQ ID NO: 178 (FIG. 194). The amino acid sequence of C3022-C3E7036 is described in SEQ ID NO: 179 (FIG. 195).

### Example 13

### In Vitro Activity Evaluation of Anti-GPRC5D-Anti-CD3 Bispecific Molecule

### 13)-1 Binding Activity Evaluation of Anti-GPRC5D-Anti-CD3 Bispecific Molecule by Flow Cytometry

### 13)-1-1 Binding of Anti-GPRC5D-Anti-CD3 Bispecific Molecule to Endogenous Human GPRC5D-Expressing Cell (A4/Fuk)

Lymphoma cell line A4/Fuk cells (JCRB Cell Bank) were adjusted to an appropriate concentration with PBS containing 5% FBS. LIVE/DEAD Fixable Near-IR Dead Cell Stain Kit was added to the cells, which were then left standing at 4° C. for 30 minutes. The cells were washed twice with PBS containing 5% FBS, then adjusted to a concentration of 1×10⁶ cells/mL with PBS containing 5% FBS, inoculated in an amount of 100 μL/well to a 96-well U-bottomed microplate, and centrifuged to remove a supernatant. Each anti-GPRC5D-anti-CD3 bispecific molecule (C2037-C3E7034 to C3022-C3E7036 prepared in Example 12) diluted with PBS containing 5% FBS were added thereto in an amount of 100 μL/well, and the plate was left standing at 4° C. for 60 minutes. The cells were washed twice with PBS containing 5% FBS. Then, Penta-His Alexa Fluor 488 diluted with PBS containing 5% FBS was added thereto in an amount of 30 μL/well, and the plate was left standing at 4° C. for 30 minutes. The cells were washed twice with PBS containing 5% FBS and then resuspended in PBS containing 5% FBS, followed by detection using a flow cytometer (FACSCanto™ II). The data was analyzed using Flowjo. The mean fluorescence intensity (MFI) of Alexa Fluor 488 in a fraction free from dead cells was calculated. The MFI value of the antibody-unsupplemented sample was subtracted from the MFI value of the antibody-supplemented sample to calculate a relative value of MFI (rMFI). As shown in FIG. 196, these anti-GPRC5D-anti-CD3 bispecific antibodies were found to bind to endogenous human GPRC5D-expressing cells.

### 13)-1-2 Binding of Anti-GPRC5D-Anti-CD3 Bispecific Molecule to Cynomolgus Monkey GPRC5D-Expressing Cells

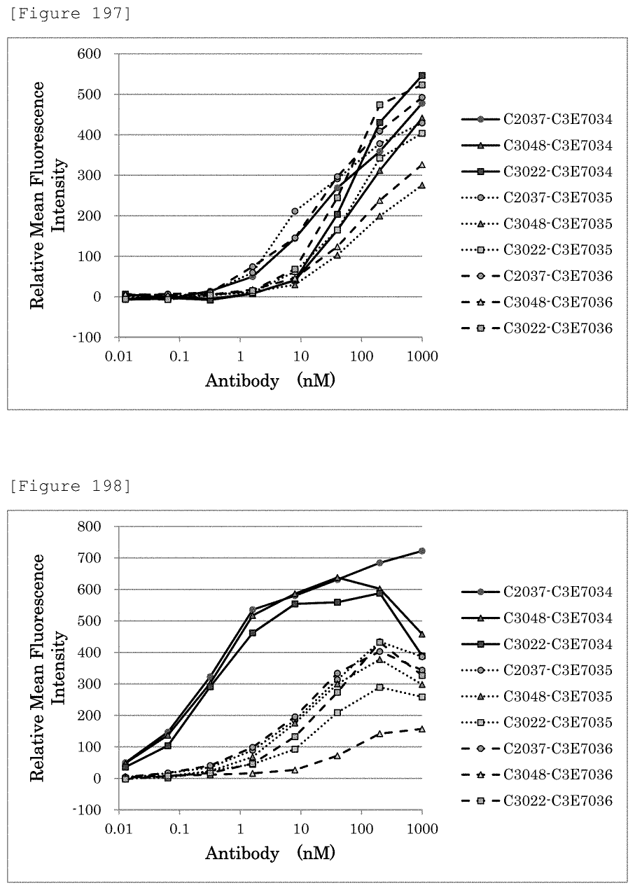

Staining and analysis were carried out in the same way as in Example 13)-1-1 using the KMS-11_cGPRC5D cells prepared in Example 5)-2-2. As shown in FIG. 197, these anti-GPRC5D-anti-CD3 bispecific antibodies were found to bind to cynomolgus monkey GPRC5D-expressing cells.

### 13)-1-3 Binding of Anti-GPRC5D-Anti-CD3 Bispecific Molecule to Human CD3 (PBMC)

Commercially available human PBMC (Cellular Technology Limited) was adjusted to an appropriate concentration with PBS containing 5% FBS. LIVE/DEAD Fixable Near-IR Dead Cell Stain Kit (Thermo Fisher Scientific Inc.) and an anti-CD19 antibody (Beckman Coulter Inc.) were added to the cells, which were then left standing at 4° C. for 30 minutes. The cells were washed twice with PBS containing 5% FBS, then adjusted to a concentration of 1×10⁶ cells/mL with PBS containing 5% FBS, inoculated in an amount of 100 μL/well to a 96-well U-bottomed microplate, and centrifuged to remove a supernatant. Each anti-GPRC5D-anti-CD3 bispecific molecule (C2037-C3E7034 to C3022-C3E7036 prepared in Example 12) diluted with PBS containing 5% FBS were added thereto in an amount of 100 μL/well, and the plate was left standing at 4° C. for 60 minutes. The cells were washed twice with PBS containing 5% FBS. Then, Penta-His Alexa Fluor 488 (Qiagen N.V.) diluted with PBS containing 5% FBS was added thereto in an amount of 30 μL/well, and the plate was left standing at 4° C. for 30 minutes. The cells were washed twice with PBS containing 5% FBS and then resuspended in PBS containing 5% FBS, followed by detection using a flow cytometer (FACSCanto™ II; Becton, Dickinson and Company). The data was analyzed using Flowjo (Tree Star, Inc.). The mean fluorescence intensity (MFI) of Alexa Fluor 488 in a fraction free from dead cells and CD19-positive cells was calculated. The MFI value of the antibody-unsupplemented sample was subtracted from the MFI value of the antibody-supplemented sample to calculate a relative value of MFI (rMFI). As shown in FIG. 198, these anti-GPRC5D-anti-CD3 bispecific antibodies were found to bind to human CD3-expressing cells.

### 13)-1-4 Binding of Anti-GPRC5D-Anti-CD3 Bispecific Molecule to Cynomolgus Monkey CD3 (PBMC)

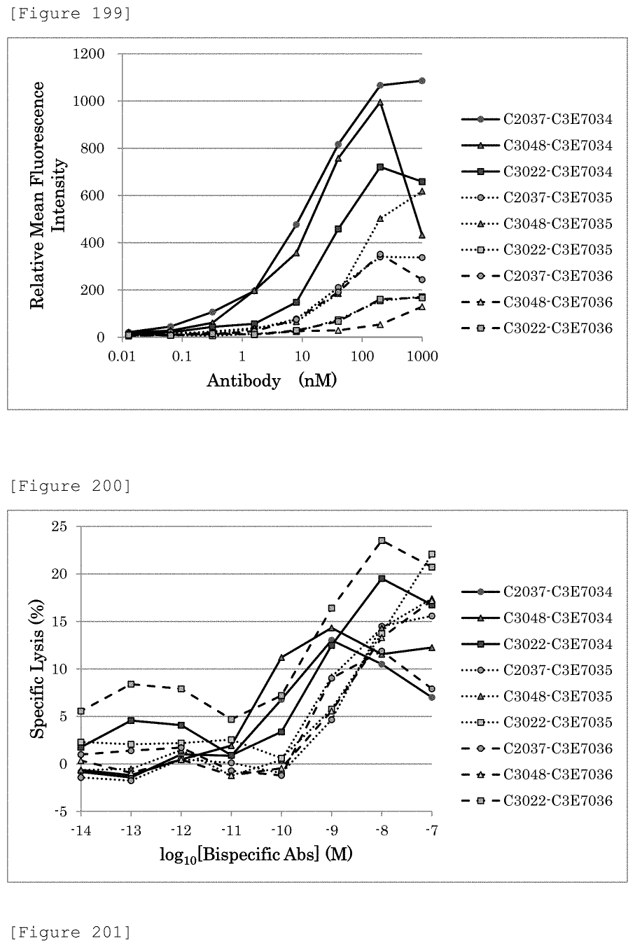

PBMC was collected from the blood of a cynomolgus monkey according to a standard method using SepMate (StemCell Technologies Inc.) and Lymphocyte Separation Solution (Nacalai Inc.). Using the collected cynomolgus monkey PBMC, staining and analysis were carried out in the same way as in Example 13)-1-3. As shown in FIG. 199, these anti-GPRC5D-anti-CD3 bispecific antibodies were found to bind to cynomolgus monkey CD3-expressing cells.

### 13)-2 Cytotoxic Activity Evaluation of Anti-GPRC5D-Anti-CD3 Bispecific Molecule

### 13)-2-1 Preparation of Target Cell

A4/Fuk cells were adjusted to a concentration of 1×10⁶ cells/mL with an RPMI1640 medium (Thermo Fisher Scientific Inc.) containing 10% FBS. 100 μL of Chromium-51 Radionuclide (PerkinElmer, Inc.) was added per mL of the cell suspension, and the cells were cultured at 37° C. for 2 hours under 5% CO₂ conditions. The cells were washed twice with an RPMI1640 medium containing 10% FBS, then resuspended to 1×10³ cells/mL in an RPMI1640 medium containing 10% FBS, and used as target cells.

### 13)-2-2 Preparation of Effector Cell

Commercially available frozen PBMC (Cellular Technology Limited) was thawed at 37° C., transferred to a solution of an RPMI1640 medium containing 10% FBS supplemented with Anti-aggregate Wash reagent (Cellular Technology Limited), washed twice, then adjusted to 1×10⁶ cells/mL with an RPMI1640 medium containing 10% FBS, and used as effector cells.

### 13)-2-3 Cytotoxicity Assay

The A4/Fuk cells obtained in Example 13)-2-1 were added at a concentration of 50 μL/well to a 96-well U-bottomed microplate. Each anti-GPRC5D-anti-CD3 bispecific molecule (prepared in Example 12) adjusted to varying concentrations was added thereto in an amount of 50 μL/well. The effector cells prepared in Example 13)-2-2 were added thereto in an amount of 100 μL/well. After centrifugation at room temperature at 1000 rpm for 1 minute, the cells were cultured at 37° C. for 20 to 24 hours under 5% CO₂ conditions. A 50 μL aliquot of the supernatant was recovered into LumaPlate (PerkinElmer, Inc.) and dried at 50° C. for approximately 2 hours, followed by measurement using a plate reader (TopCount; PerkinElmer, Inc.). The percentage of cells lysed was calculated according to the following expression:

Percentage of cells lysed (%)=(A−B)/(C−B)×100
A: Count of sample well
B: Average of background (antibody-unsupplemented wells) counts (n=3). 50 μL of a medium for assay was added instead of adding the antibody. The other procedures were the same as in the case of the sample well.
C: Average of maximum release (wells containing target cells lysed in a surfactant) counts (n=3). 50 μL of a medium for assay was added instead of adding the antibody. 100 μL of the surfactant was added, and the 50 μL aliquot was transferred to LumaPlate, as with the sample well, and assayed.

As shown in FIG. 200, these anti GPRC5D-anti-CD3 bispecific antibodies exhibited cytotoxic activity against the A4/Fuk cells.

### Example 14

### Preparation of Fc-Containing Anti-GPRC5D-Anti-CD3 Bispecific Molecule

### 14)-1 Preparation of Fc-Containing Anti-GPRC5D-Anti-CD3 Bispecific Molecule Expression Vector

### 14)-1-1 Preparation of Full-Size Antibody (FSA)-Type Bispecific Molecule Expression Vector

DNA encoding the humanized anti-GPRC5D antibody (h2B1) H2 type heavy chain variable region constructed in Example 8)-1-2 was net-synthesized artificially (Genscript Custom Gene Synthesis Service). Expression vectors “pCL_#13540” and “pCL_#13543” were prepared by inserting DNAs encoding the obtained heavy chain variable region, two types of human IgG-derived CH1 regions, and a Fc region with reduced effector functions and containing heteromultimer-forming mutations (WO2014/190441) to mammalian expression vector pTT5 (National Research Council, WO2009/137911). Also, DNA encoding the humanized anti-GPRC5D antibody (h2B1) L5 type light chain variable region constructed in Example 8)-2-5 was net-synthesized artificially. The expression vectors “pCL_#12290” and “pCL_#12313” were prepared by inserting DNAs encoding the obtained light chain variable region and two types of human IgG-derived CL regions to mammalian expression vector pTT5.

Next, a DNA fragment encoding the heavy chain of the humanized anti-CD3 scFv (C3E-7034) constructed in Example 11)-2 was net-synthesized artificially. “pCL_#13552” was prepared by inserting DNAs encoding the obtained scFv heavy chain variable region, human IgG-derived CH1, and a Fc region with reduced effector functions and containing heteromultimer-forming mutations to mammalian expression vector pTT5. Also, a DNA fragment encoding the light chain of the humanized anti-CD3 scFv (C3E-7034) constructed in Example 11)-2 was net-synthesized artificially. “pCL_#12287” was prepared by inserting DNAs encoding the obtained scFv light chain variable region and human IgG-derived CL to mammalian expression vector pTT5. Likewise, a DNA fragment encoding the heavy chain of the humanized anti-CD3 scFv (C3E-7036) constructed in Example 11)-4 was net-synthesized. “pCL_#13541” was prepared by inserting DNA sequences encoding the obtained scFv heavy chain variable region, human IgG-derived CH1, and a Fc region with reduced effector functions and containing heteromultimer-forming mutations to mammalian expression vector pTT5. Also, a DNA fragment encoding the light chain of the humanized anti-CD3 scFv (C3E-7036) constructed in Example 11)-4 was net-synthesized. “pCL_#12321” was prepared by inserting DNA fragments encoding the obtained scFv light chain variable region and human IgG-derived CL to mammalian expression vector pTT5.

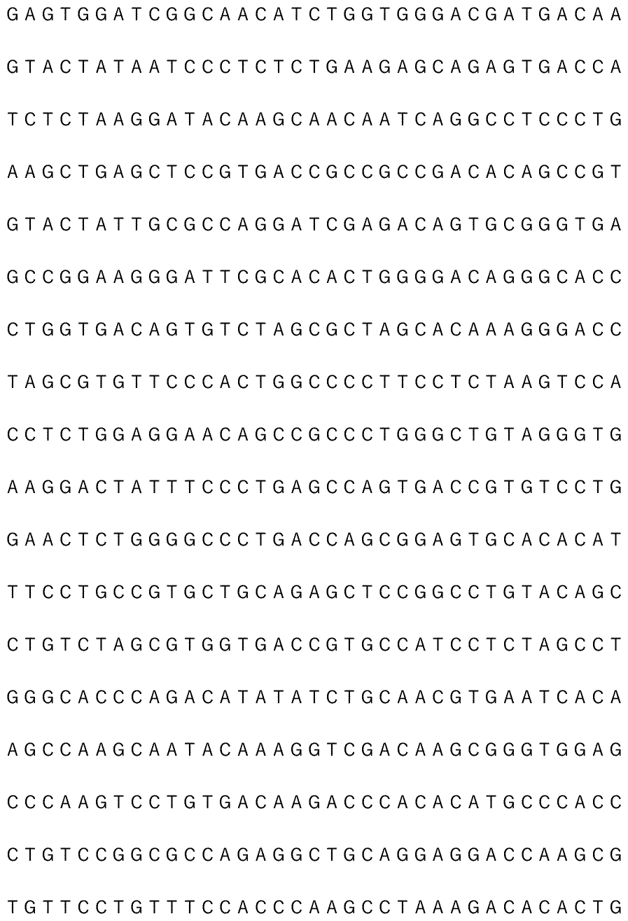

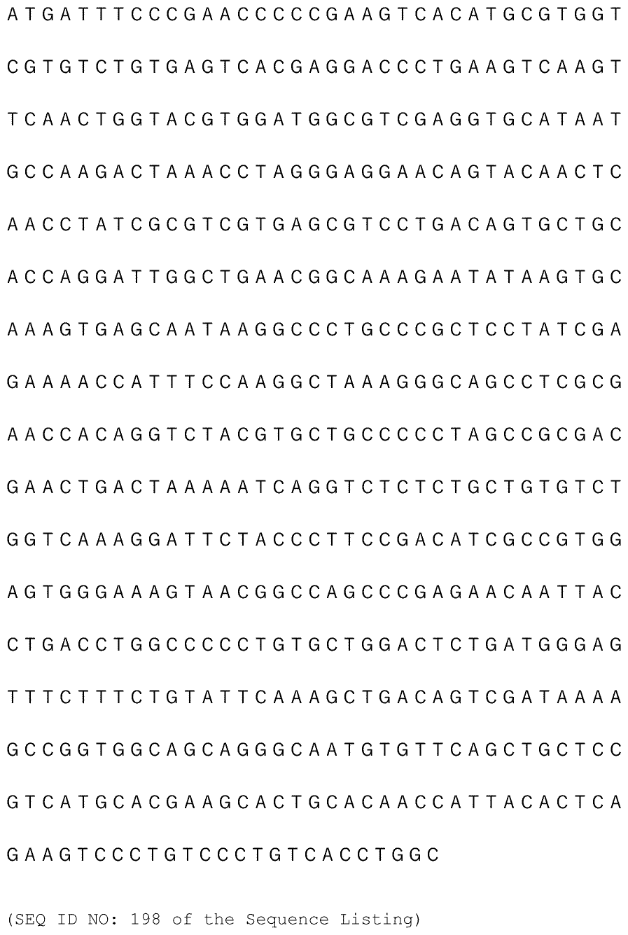

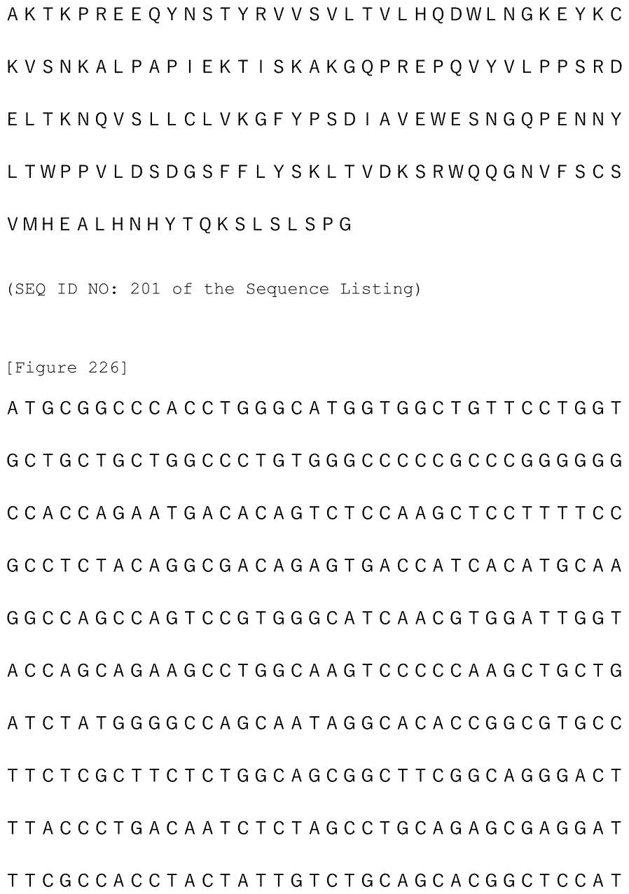

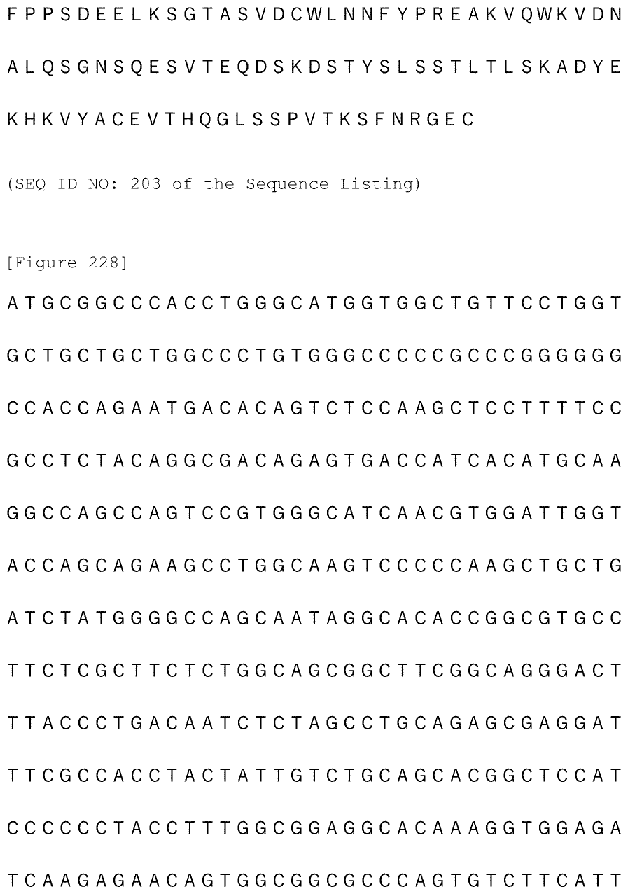

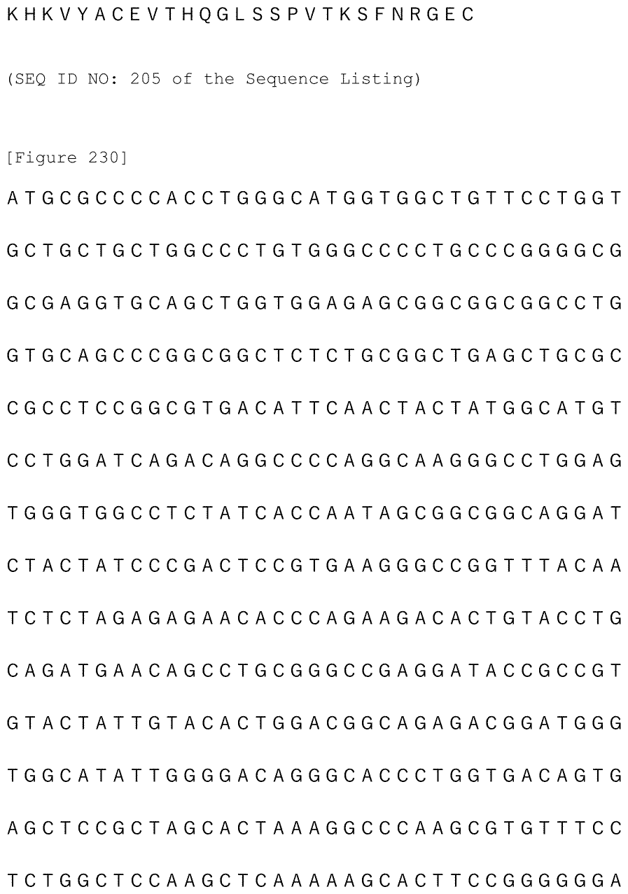

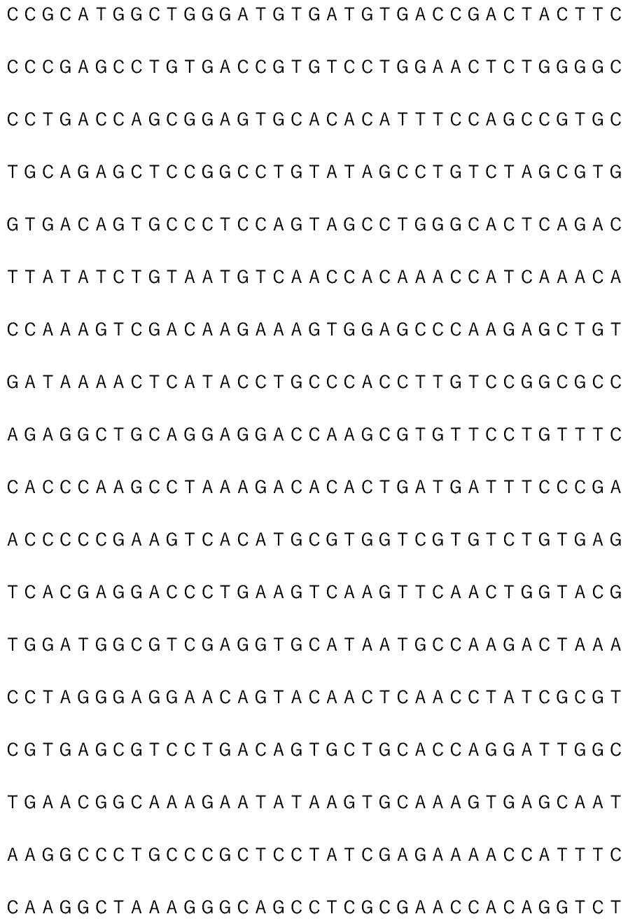

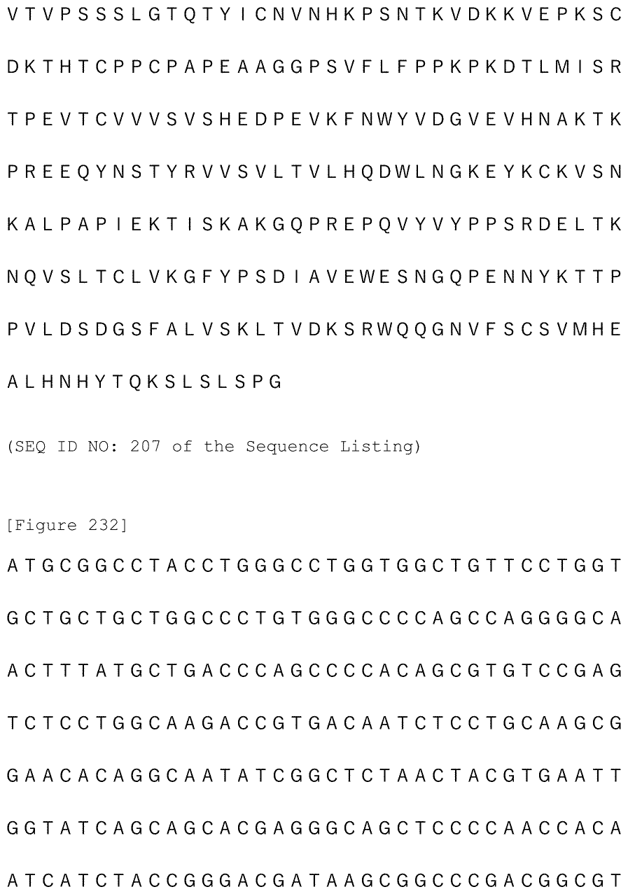

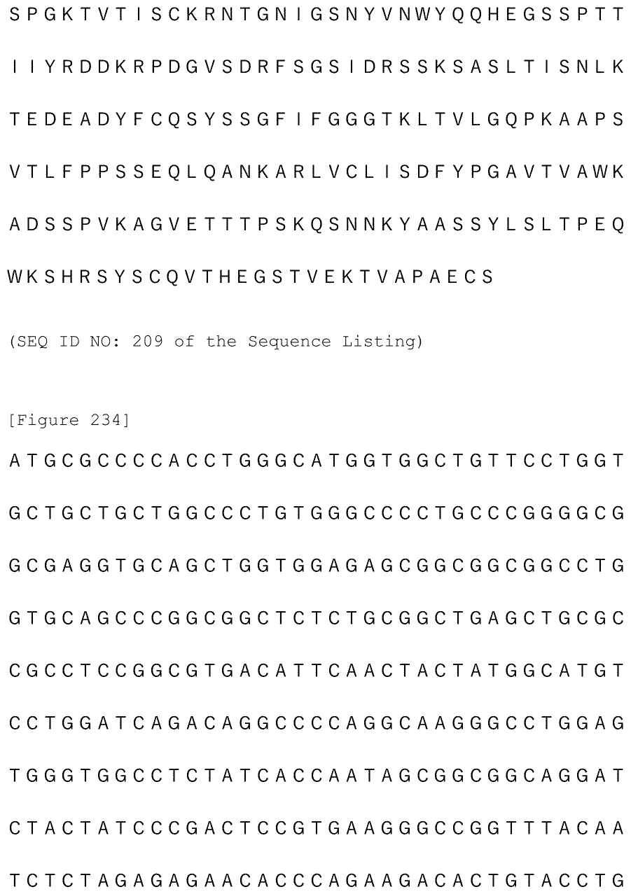

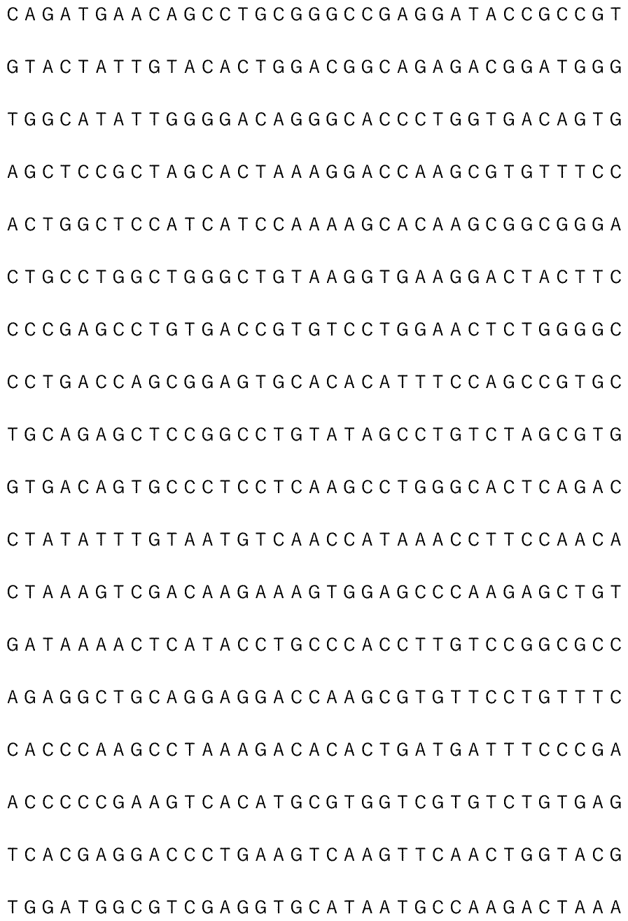

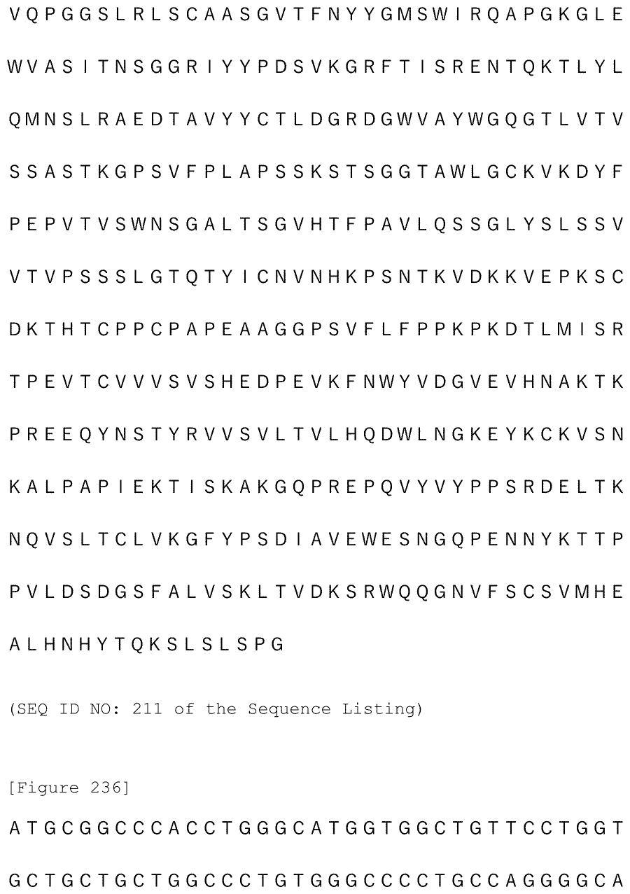

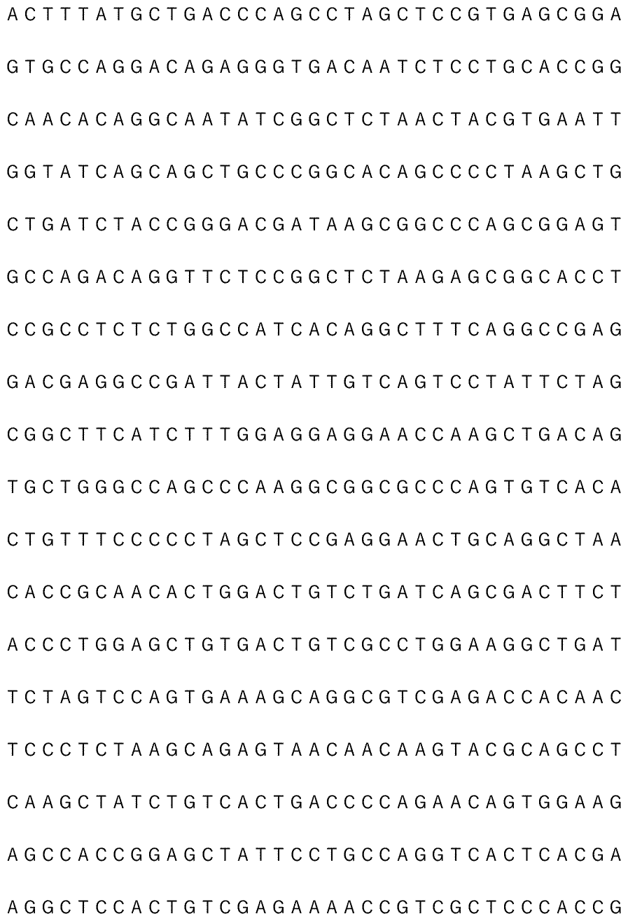

The ORF sequences of pCL_#13540, pCL_#13543, pCL_#12290, pCL_#12313, pCL_#13552, pCL_#12287, pCL_#13541, and pCL_#12321 are shown in SEQ ID NO: 198 (FIG. 222), SEQ ID NO: 200 (FIG. 224), SEQ ID NO: 202 (FIG. 226), SEQ ID NO: 204 (FIG. 228), SEQ ID NO: 206 (FIG. 230), SEQ ID NO: 208 (FIG. 232), SEQ ID NO: 210 (FIG. 234), and SEQ ID NO: 212 (FIG. 236), respectively, of the Sequence Listing.

### 14)-1-2 Preparation of Hybrid-Type Bispecific Molecule Expression Vectors.

An expression vector for mammalian cells having an insert of DNA fragments encoding the humanized anti-GPRC5D antibody (h2B1) H2 type heavy chain variable region, a human IgG-derived CH1 region, and a Fc region with reduced effector functions and containing heteromultimer-forming mutations was prepared and designated as “pCL_#13555”. Also, an expression vector for mammalian cells having an insert of DNA fragments encoding the humanized anti-GPRC5D antibody (h2B1) L5 type light chain variable region and a human IgG-derived CL region were prepared and designated as “pCL_#12123”.

Next, an expression vector for mammalian cells having an insert of DNA fragments encoding the humanized anti-CD3 scFv (C3E-7034) and a Fc region with reduced effector functions and containing heteromultimer-forming mutations was prepared and designated as “pCL_#13557”. Also, an expression vector “pCL_#13561” for mammalian cells having an insert of DNA fragments encoding the humanized anti-CD3 scFv (C3E-7036) and a Fc region with reduced effector functions and containing heteromultimer-forming mutations was prepared.

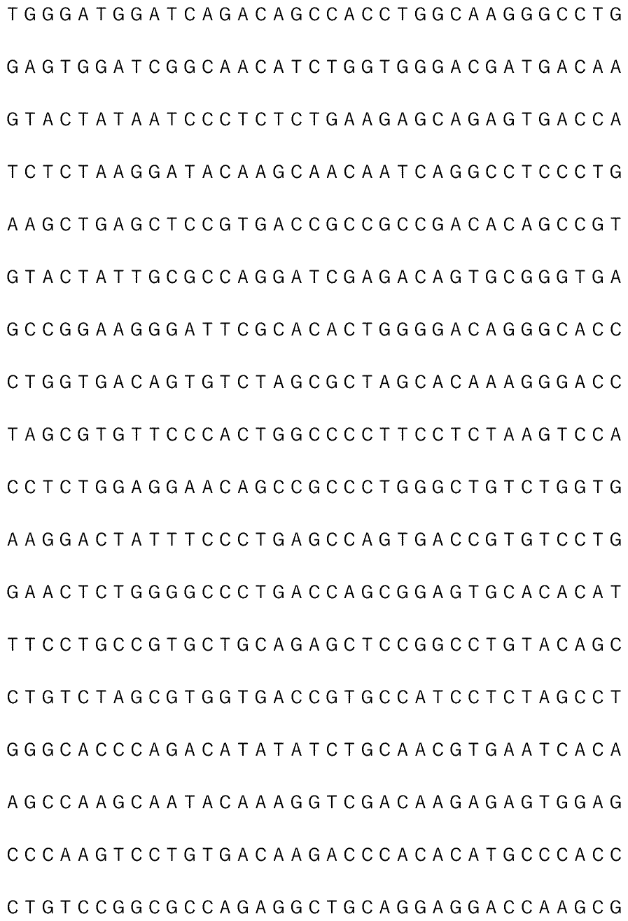

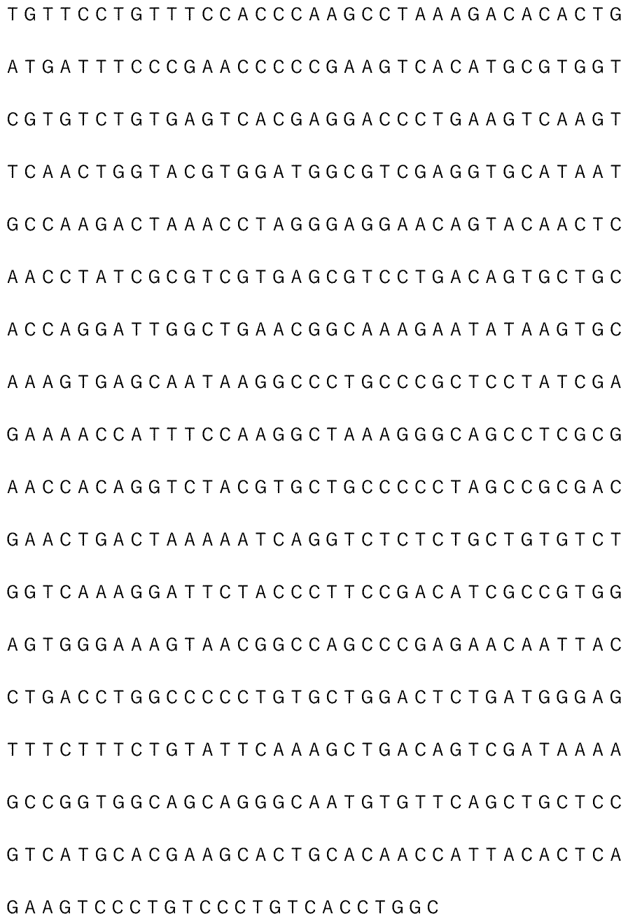

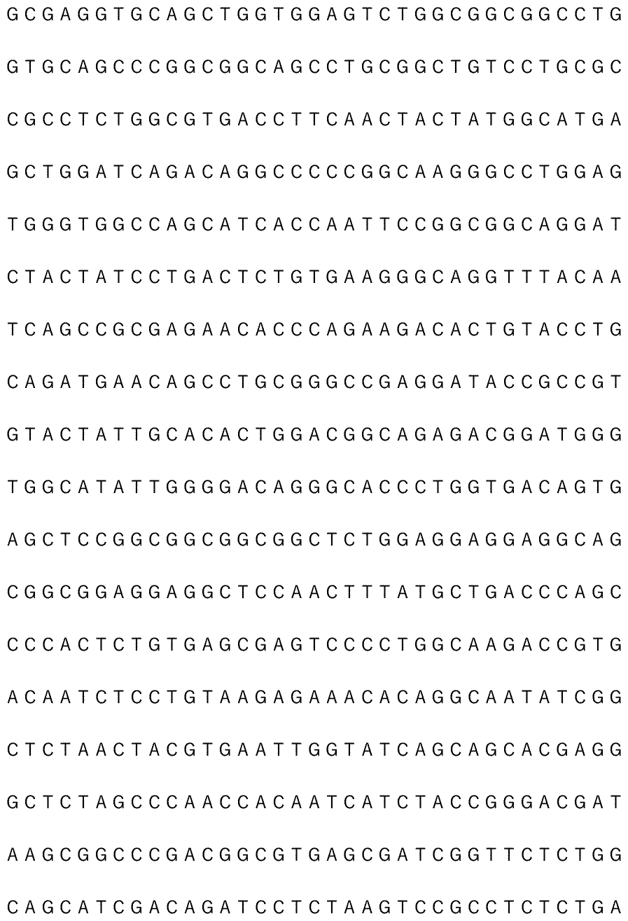

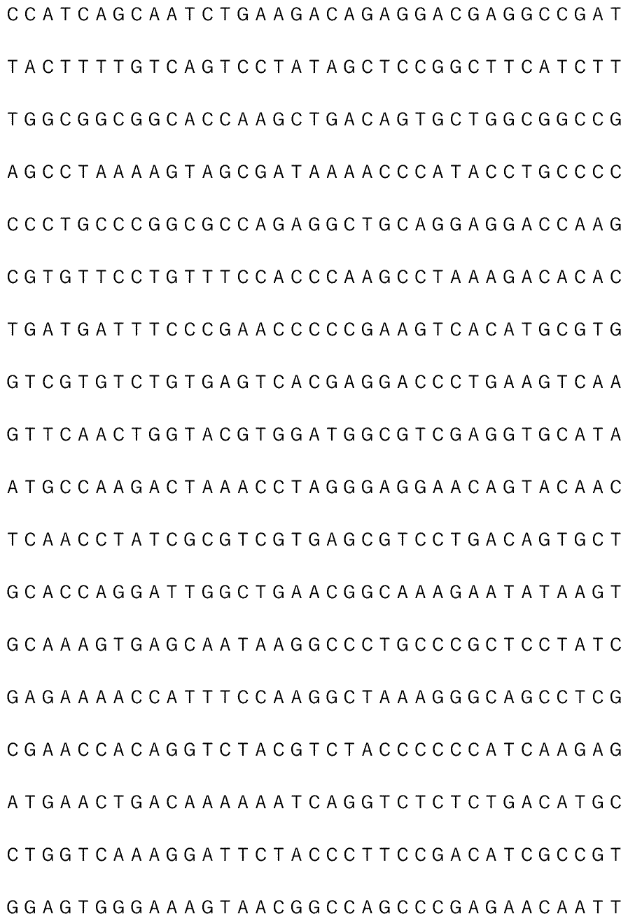

The ORF sequences of pCL_#13555, pCL_#12123, pCL_#13557, and pCL_#13561 are shown in SEQ ID NO: 214 (FIG. 238), SEQ ID NO: 216 (FIG. 240), SEQ ID NO: 218 (FIG. 242), and SEQ ID NO: 220 (FIG. 244), respectively, of the Sequence Listing.

### 14)-1-3 Preparation of Dual-Type Bispecific Molecule Expression Vectorsvector

The humanized anti-GPRC5D antibody (h2B1) H2 type heavy chain variable region and L5 type light chain variable region constructed in Example 8)-1-2 were linked via a flexible linker consisting of 3 repeat sequences of GGGGS to prepare a single chain antibody (scFv). “pCL_#13563” was prepared by inserting DNA fragments encoding this anti-GPRC5D scFv and a Fc region with reduced effector functions and containing heteromultimer-forming mutations to mammalian expression vector pTT5. The ORF sequence of pCL_#13563 is shown in SEQ ID NO: 222 (FIG. 246) of the Sequence Listing.

### 14)-2 Production of Fc-Containing Anti-GPRC5D-Anti-CD3 Bispecific Molecule

CHO-3E7 cells were subcultured and cultured according to the supplier's manual (National Research Council Canada, Raymond C. et al., Methods (2011) 55 (1), 44-51). A suspension culture of CHO-3E7 cells in the logarithmic growth phase was diluted to 2×10\hat{ }6 cells/mL with a FreeStyle F17 medium (Invitrogen Corp.) containing 4 mM glutamine and 0.1% Kolliphor (Sigma-Aldrich Corp.) and used in the production of various bispecific antibodies.

### 14)-2-1 Production of Full-Size Antibody (FSA)-Type Bispecific Molecule

8000 μg of Polyethyleneimine max (PEImax, Polysciences) was dissolved in FreeStyle F17 medium to prepare a PEImax solution. 1000 μg of a mixture of the vectors pCL_#13552, pCL_#12287, pCL_#13540, and pCL_#12290 mixed at a ratio of 15:15:53:17, or the vectors pCL_#13541, pCL_#12321, pCL_#13543, and pCL_#12313 mixed at a ratio of 22:8:17:53, was added to an aliquot of F17 medium, and 1000 μg of a DNA mixture of pAKT and pGFP (both from National Research Council) mixed with already fragmented salmon sperm DNA (Sigma-Aldrich Corp.) was added to another aliquot of F17 medium. The PEImax solution, the vector mixture, and the DNA solution were combined, gently stirred, incubated for 5 minutes, and then added to 2 L of CHO-3E7 cell suspension. The cells were shake-cultured at 37° C. for 1 day in a 5% CO2 incubator. 0.5 mM valproic acid (Sigma-Aldrich

Corp.) and 0.1% (w/v) Tryptone N1 (Organotechnie) were then added. The cells were further shake-cultured at 32° C. for 6 days. On day 7 after the start of the culture, the culture supernatant was recovered and filtered through a 0.2 μm filter (Sartorius Japan K.K.) to prepare a sample for evaluation.

pCL_#13552, pCL_#12287, pCL_#13540, and pCL_#12290 were used in the expression and preparation of a FSA-type bispecific molecule of C3E-7034 and h2B1 (v19159). pCL_#13541, pCL_#12321, pCL_#13543, and pCL_#12313 were used in the expression and preparation of a FSA-type bispecific molecule of C3E-7036 and h2B1 (v19140).

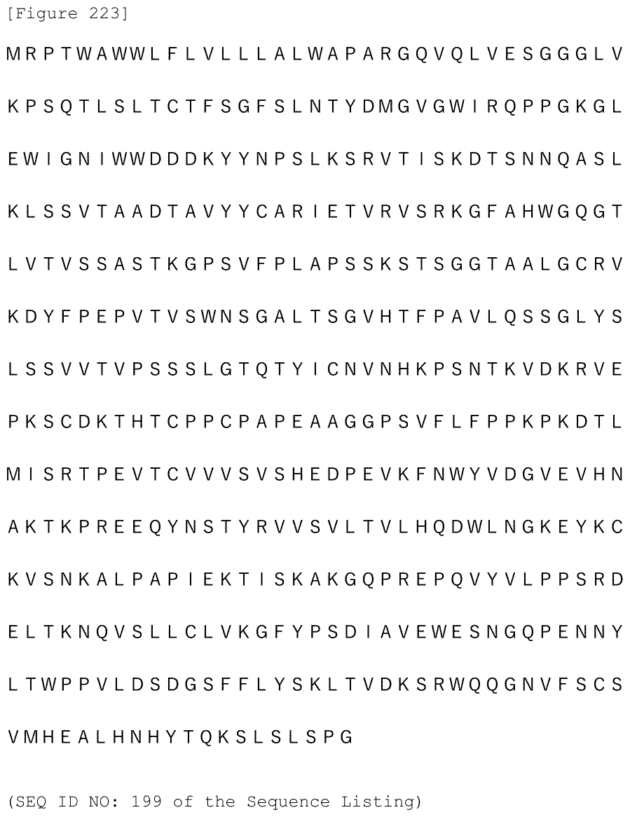

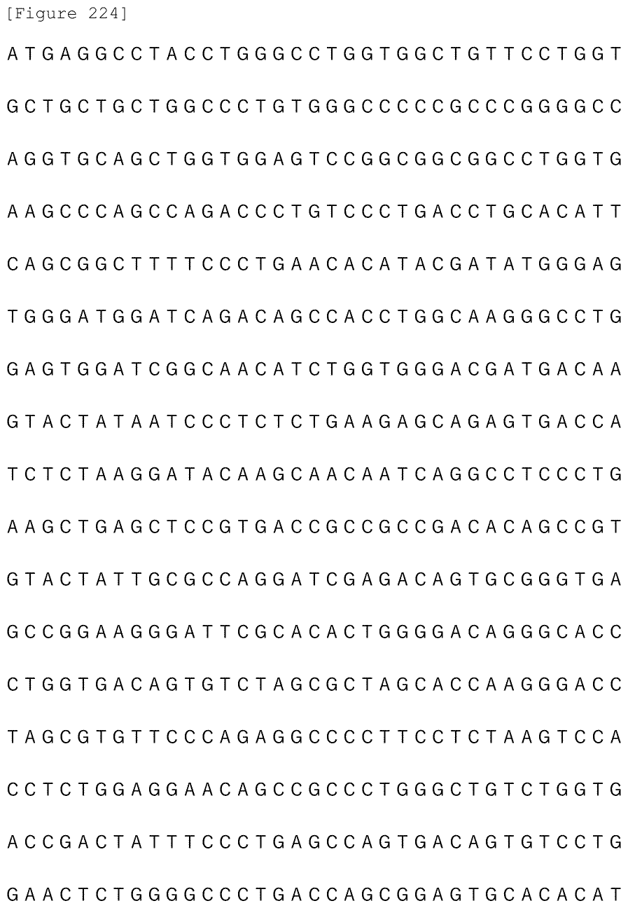

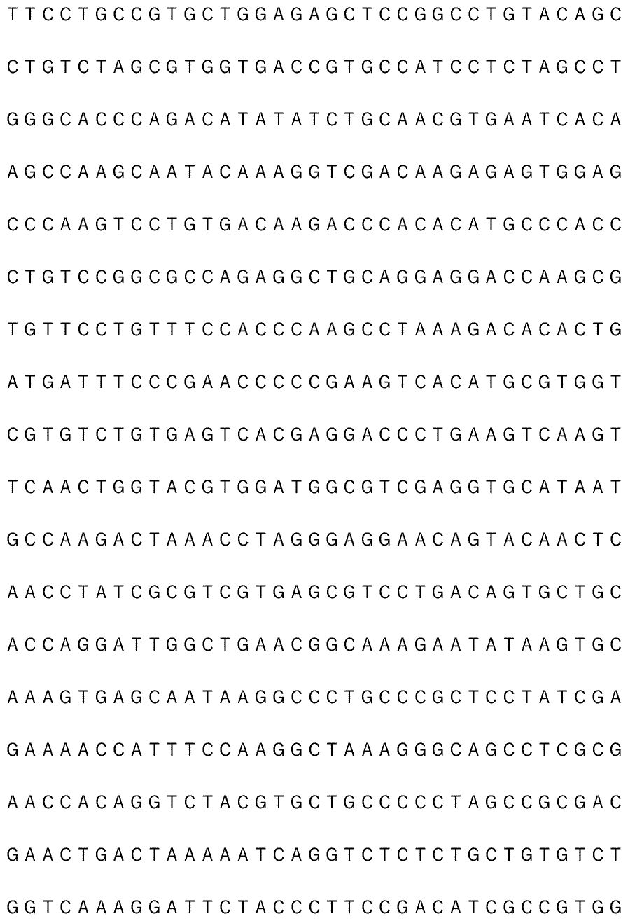

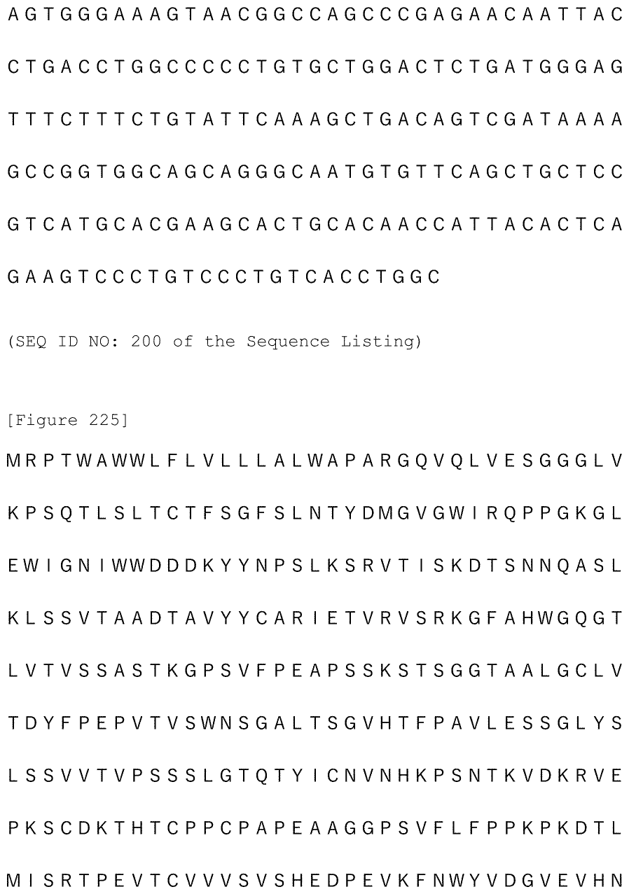

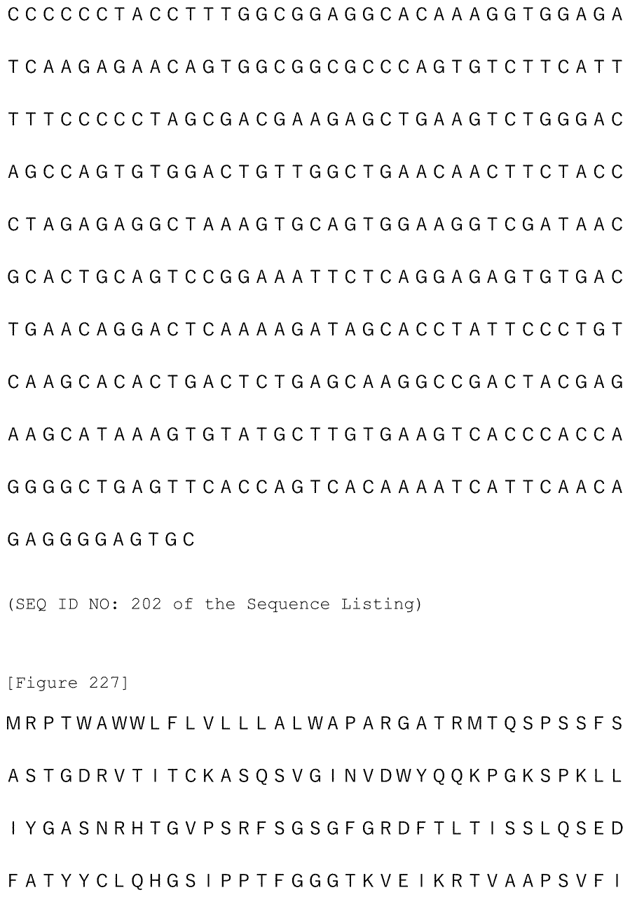

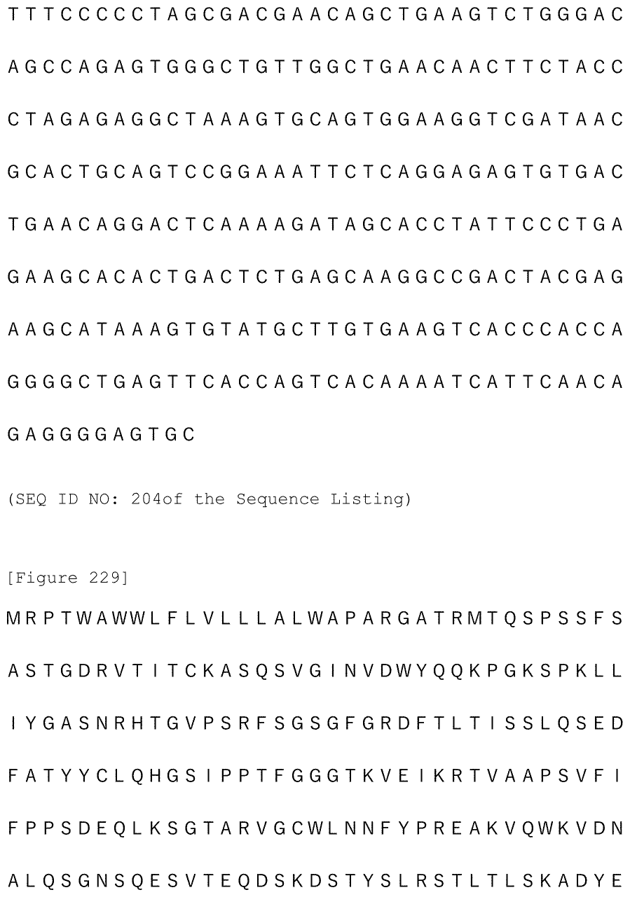

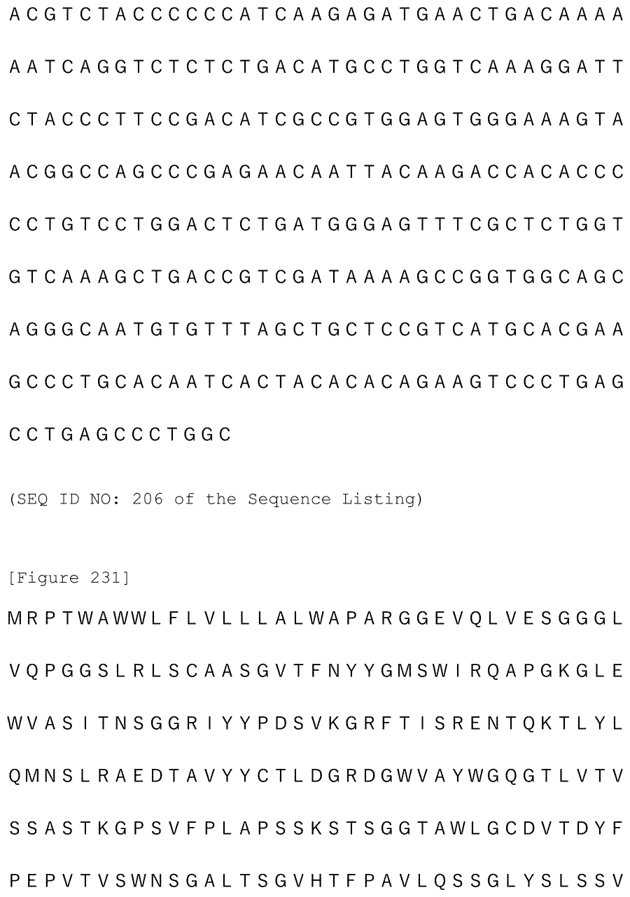

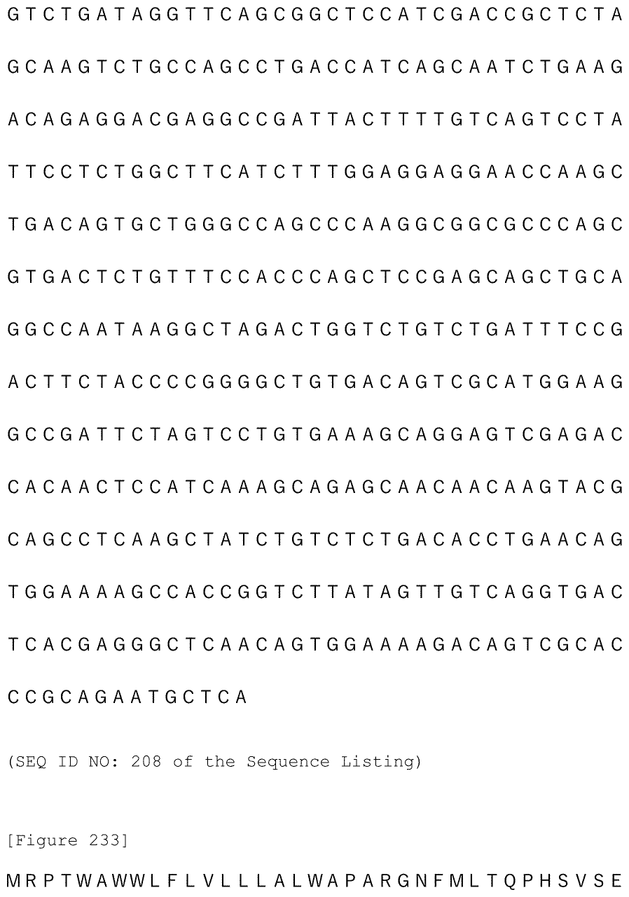

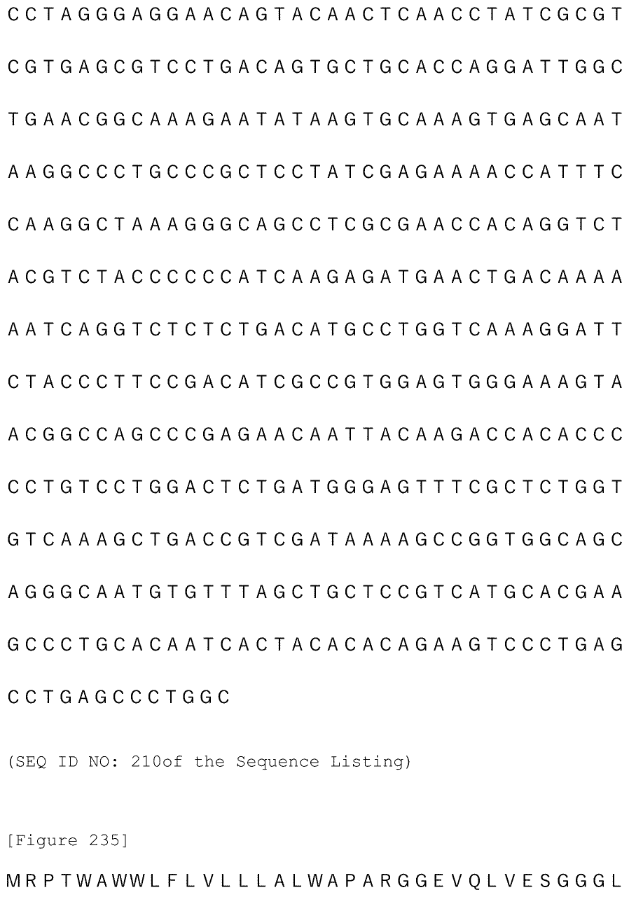

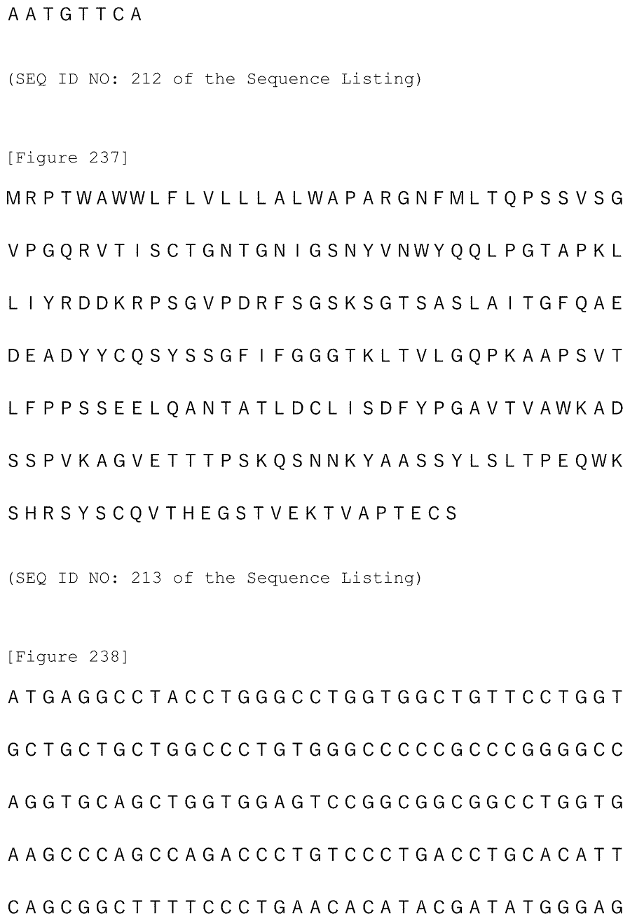

The amino acid sequences constituting v19159 obtained by expression from the respective vectors are shown in SEQ ID NOs: 207 (FIG. 231), 209 (FIG. 233), 199 (FIG. 223), and 203 (FIG. 227) of the Sequence Listing. The amino acid sequences constituting v19140 are shown in SEQ ID NOs: 211 (FIG. 235), 213 (FIG. 237), 201 (FIG. 225), and 205 (FIG. 229) of the Sequence Listing.

### 14)-2-2 Production of Hybrid-Type Bispecific Molecule

8000 μg of Polyethyleneimine max (PEImax, Polysciences) was dissolved in FreeStyle F17 medium to prepare a PEImax solution. 1000 μg of a mixture of the vectors pCL_#13557, pCL_#13555, and pCL_#12123 mixed at a ratio of 1:1:1.5, or the vectors pCL_#13561, pCL_#13555, and pCL_#12123 mixed at a ratio of 1:1:1.5, was added to an aliquot of F17 medium, and 1000 μg of a DNA mixture of pAKT and pGFP (both from National Research Council) mixed with already fragmented salmon sperm DNA (Sigma-Aldrich Corp.) was added to another aliquot of F17 medium. The PEImax solution, the vector mixture, and the pAKT/pGFP/salmon sperm DNA solution were combined, gently stirred, incubated for 5 minutes, and then added to 2 L of CHO-3E7 cell suspension. The cells were shake-cultured at 37° C. for 1 day in a 5% CO2 incubator. 0.5 mM valproic acid (Sigma-Aldrich Corp.) and 0.1% (w/v) Tryptone N1 (Organotechnie) were then added. The cells were further shake-cultured at 32° C. for 6 days. On day 7 after the start of the culture, the culture supernatant was recovered and filtered through a 0.2 μm filter (Sartorius Japan K.K.) to prepare a sample for evaluation.

pCL_#13557, pCL_#13555, and pCL_#12123 were used in the expression and preparation of a hybrid-type bispecific molecule of C3E-7034 and h2B1 (v19126). pCL_#13561, pCL_#13555, and pCL_#12123 were used in the expression and preparation of a hybrid-type bispecific molecule of C3E-7036 and h2B1 (v19125).

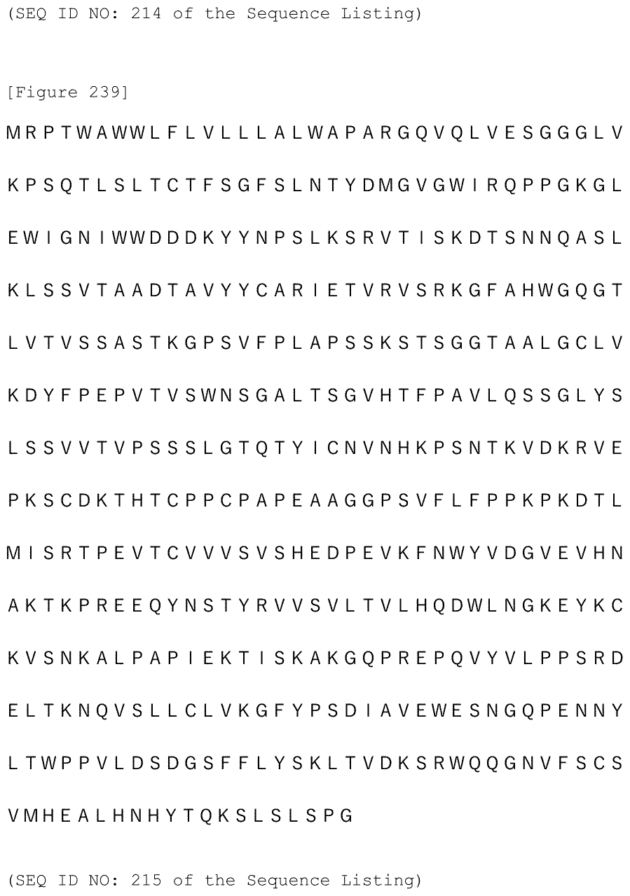

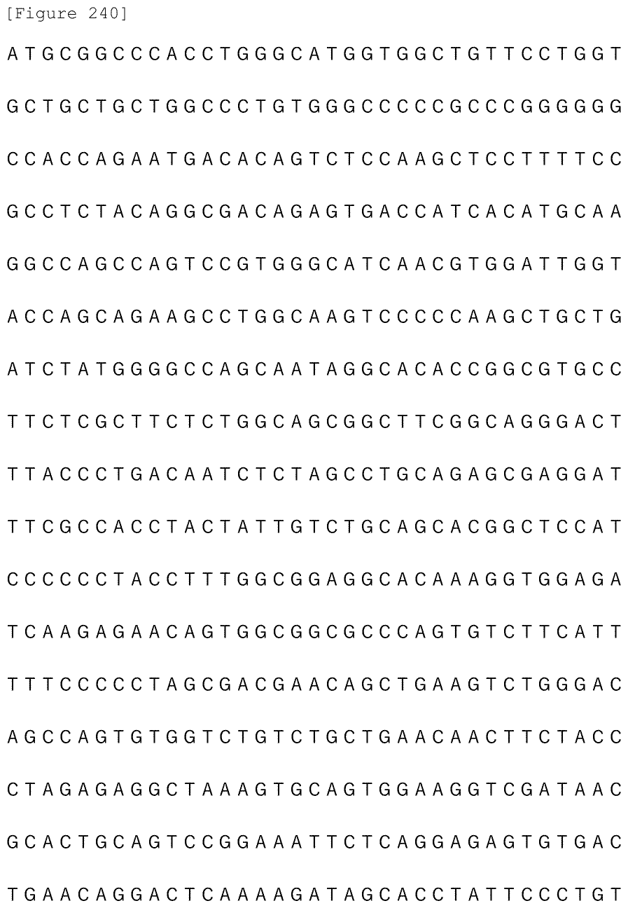

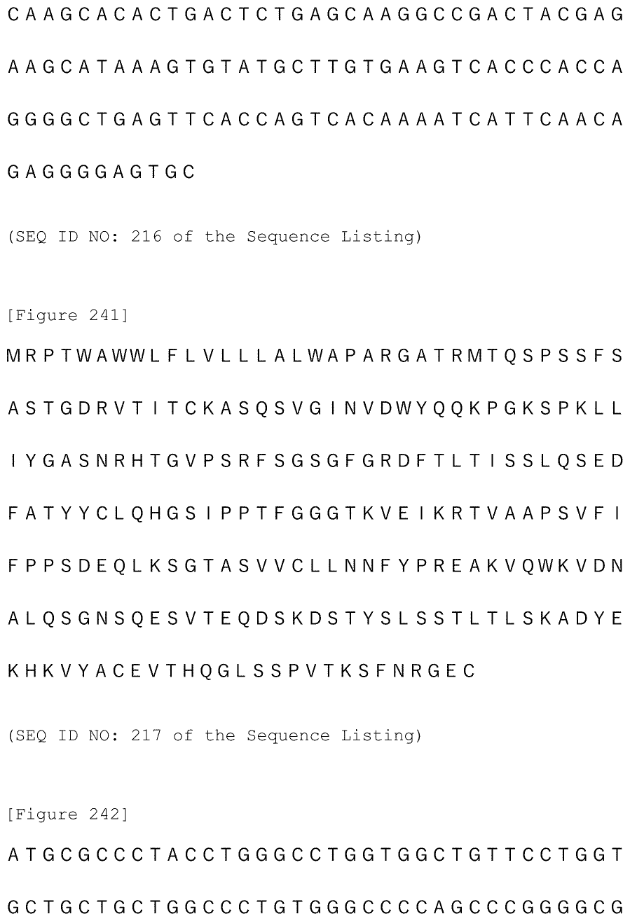

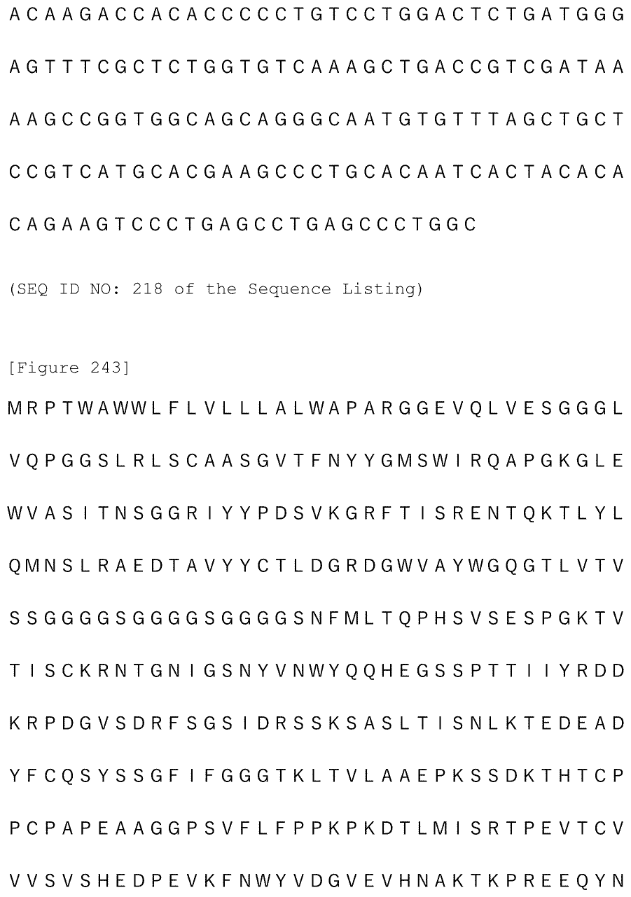

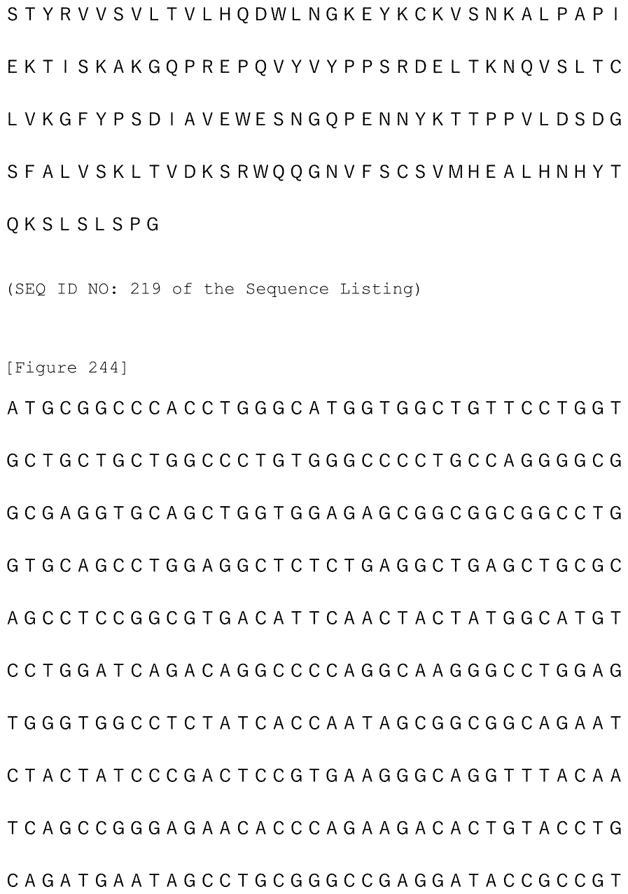

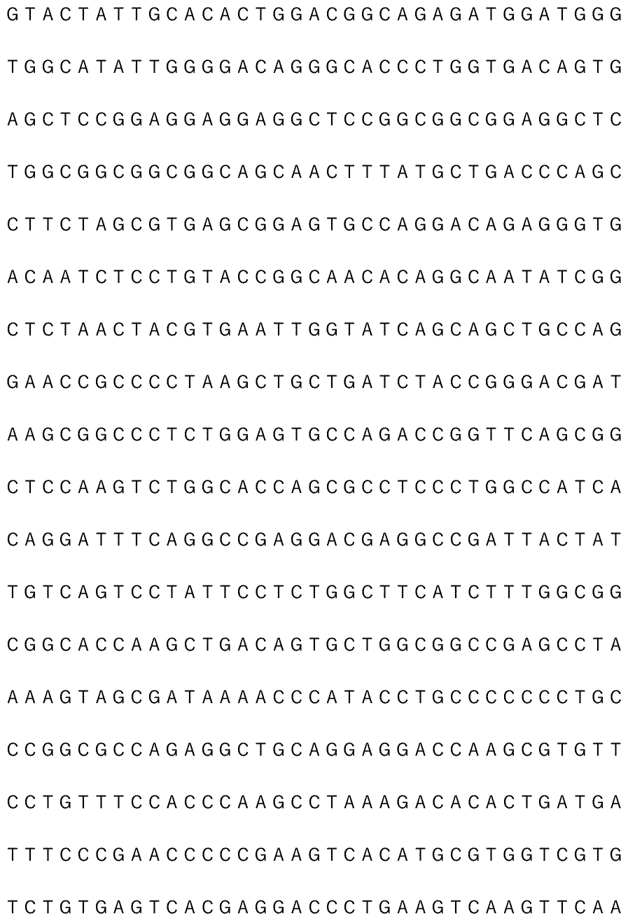

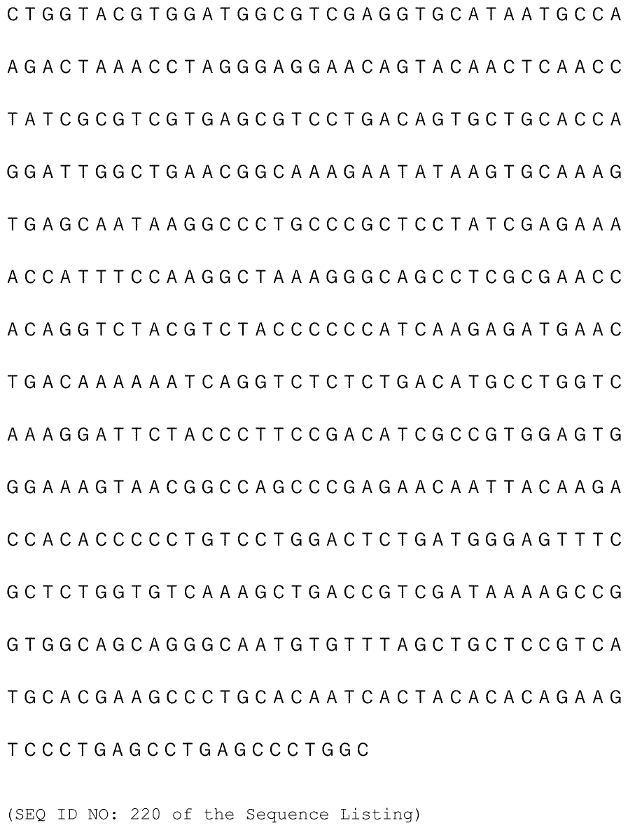

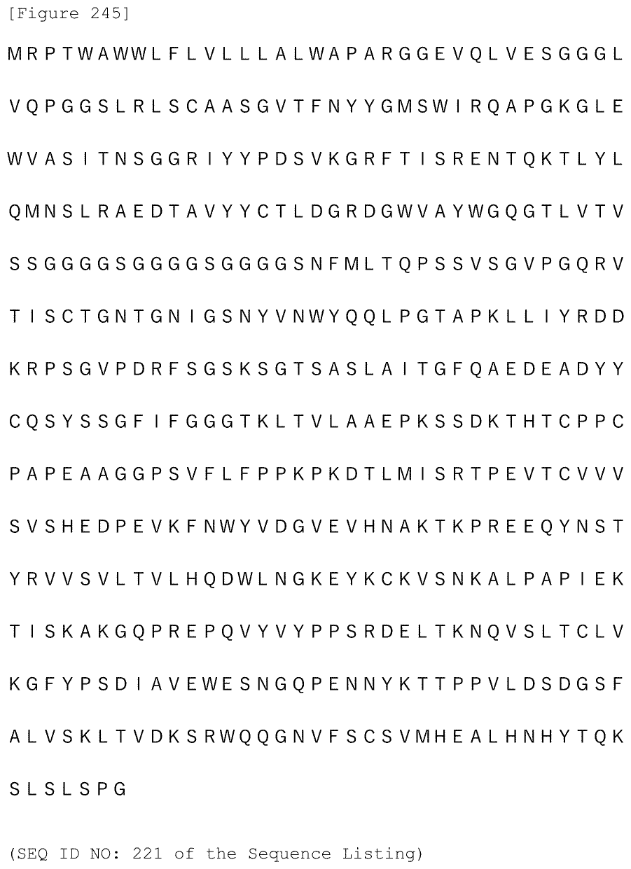

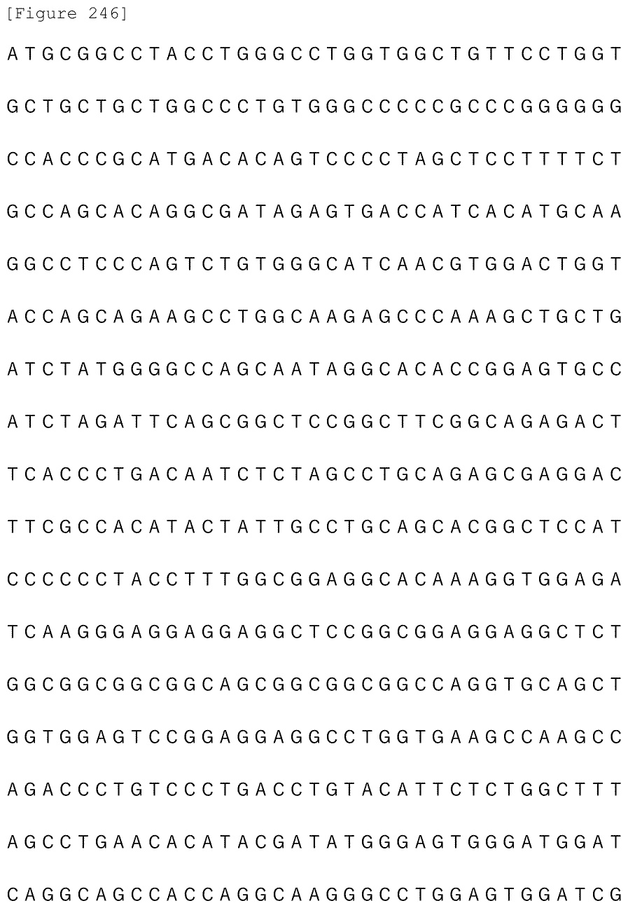

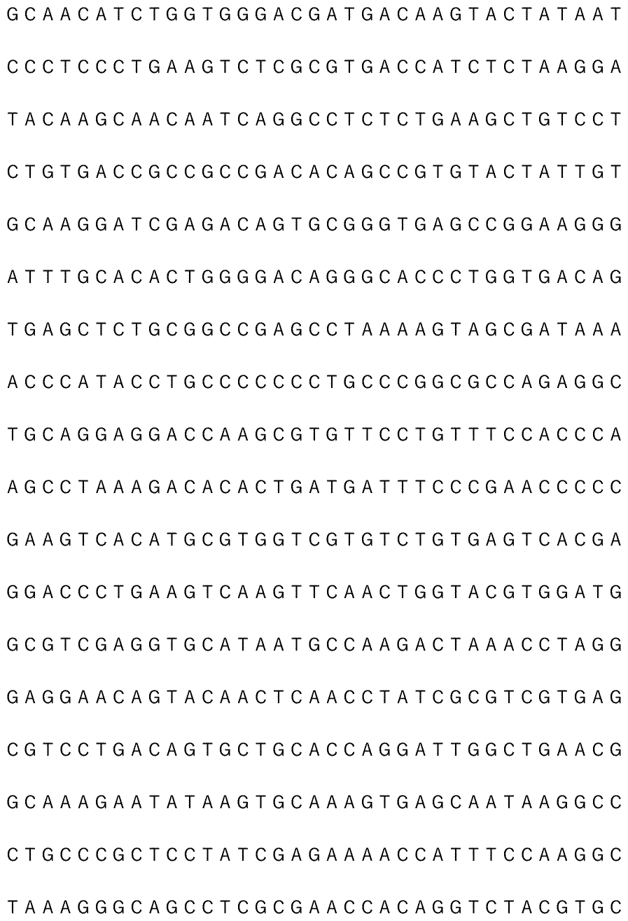

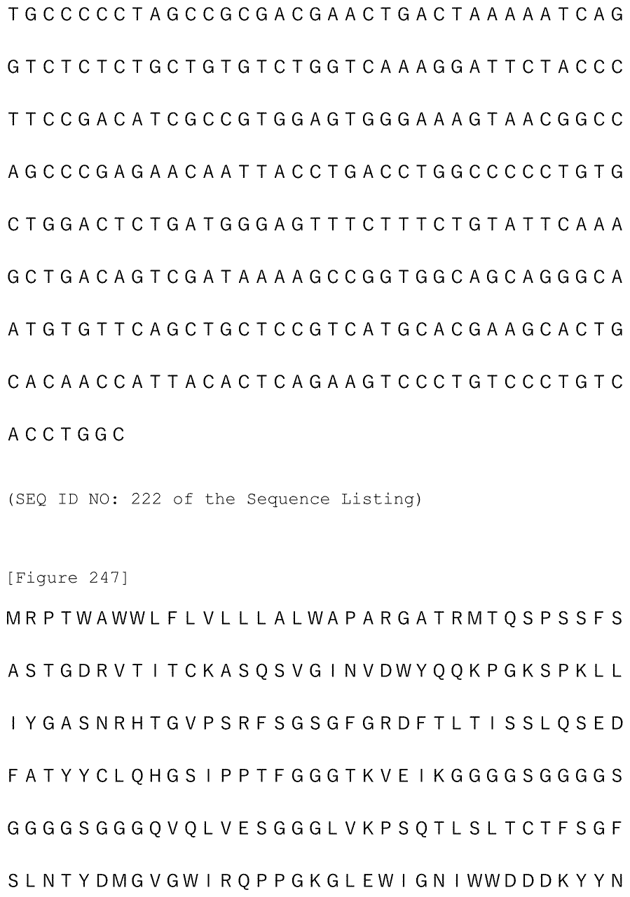

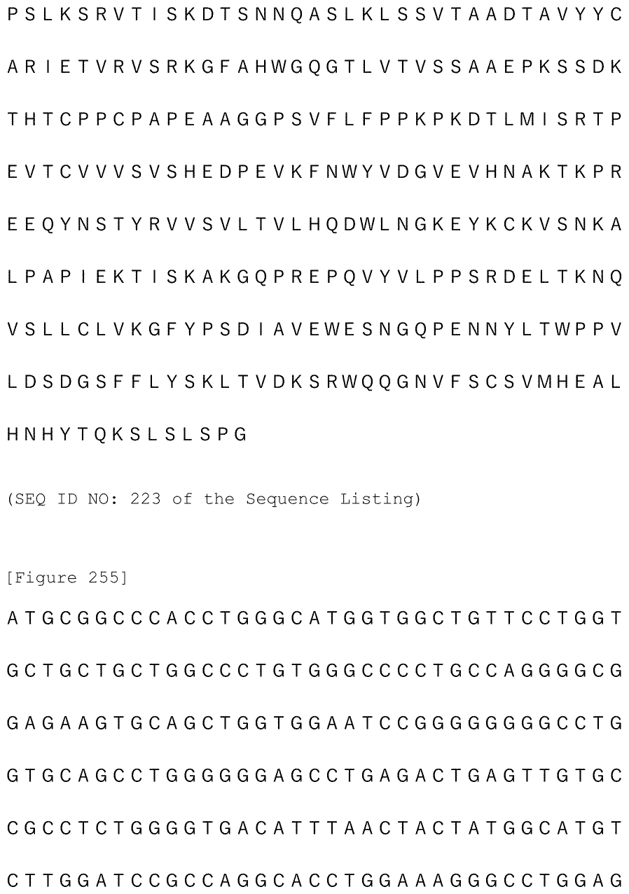

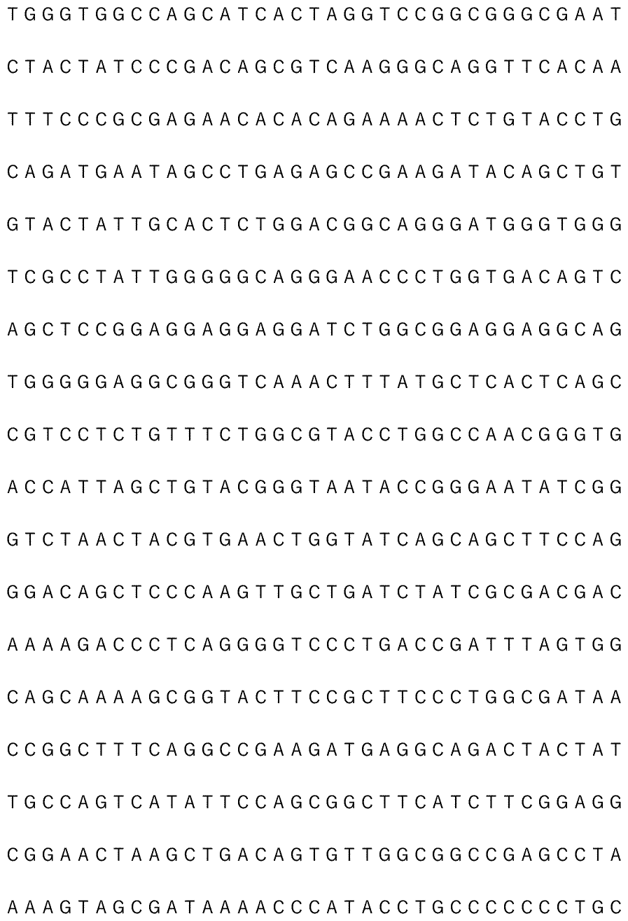

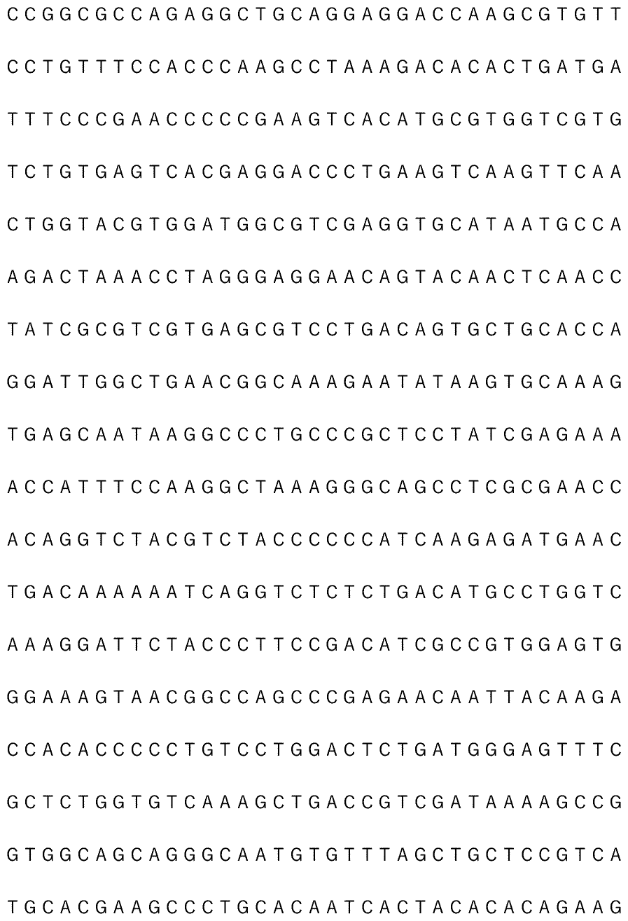

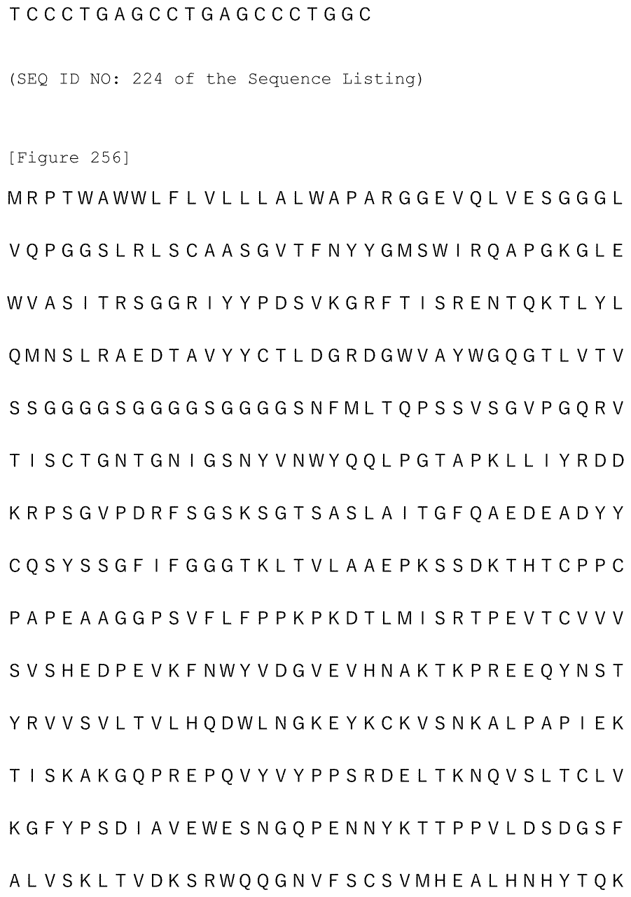

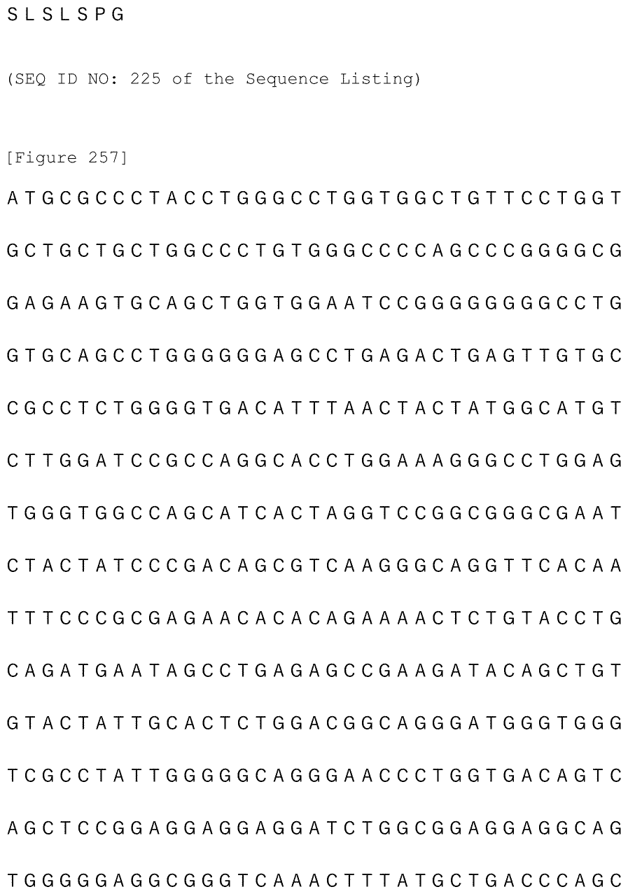

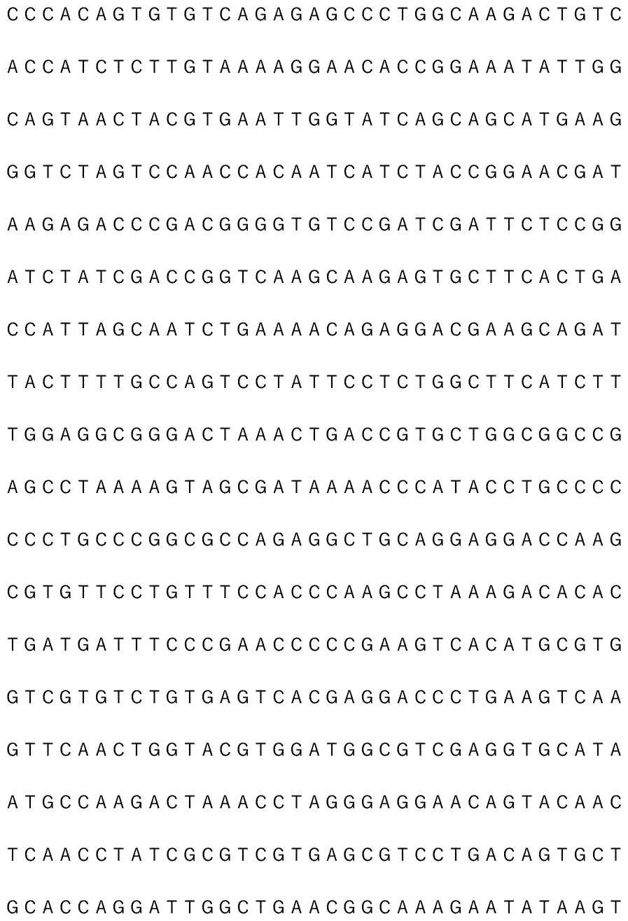

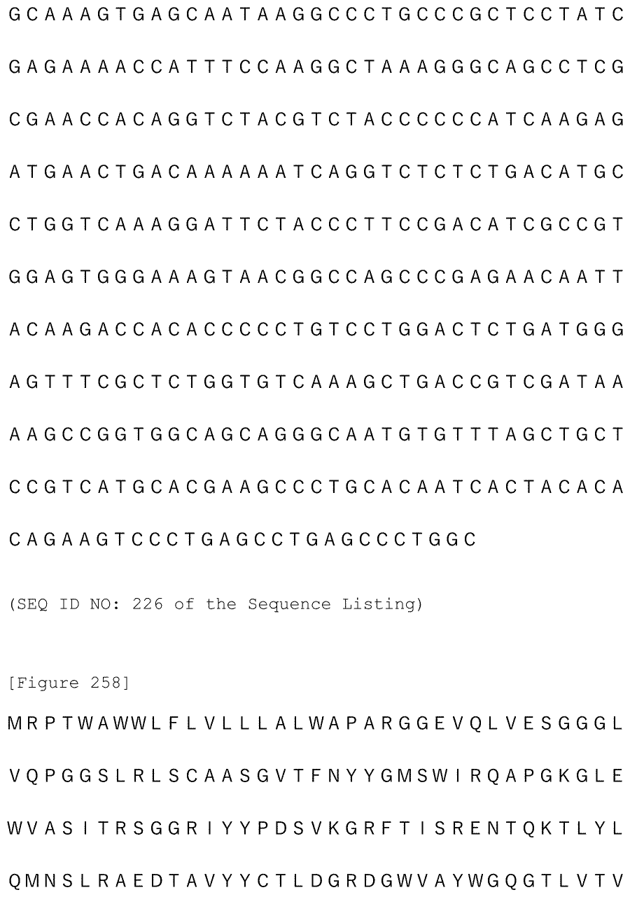

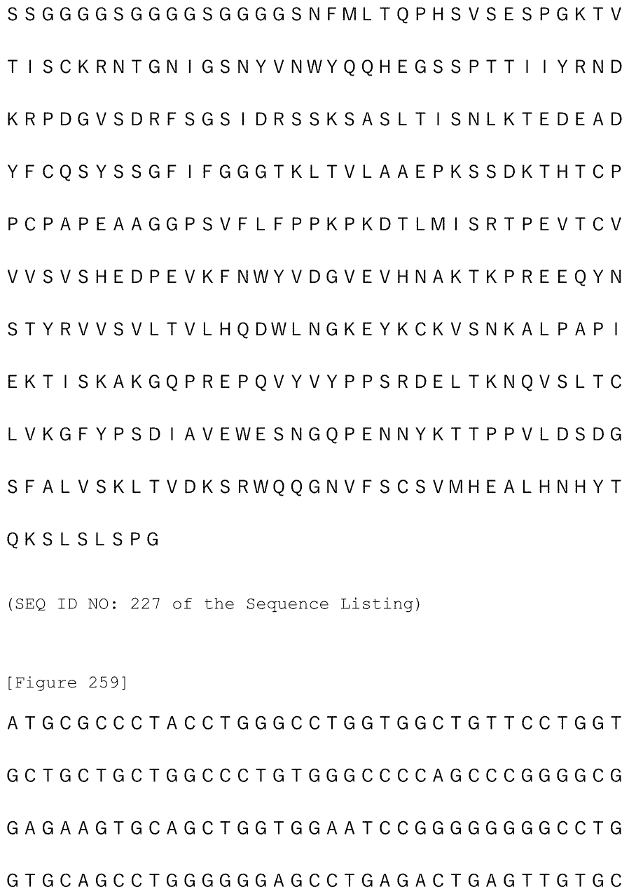

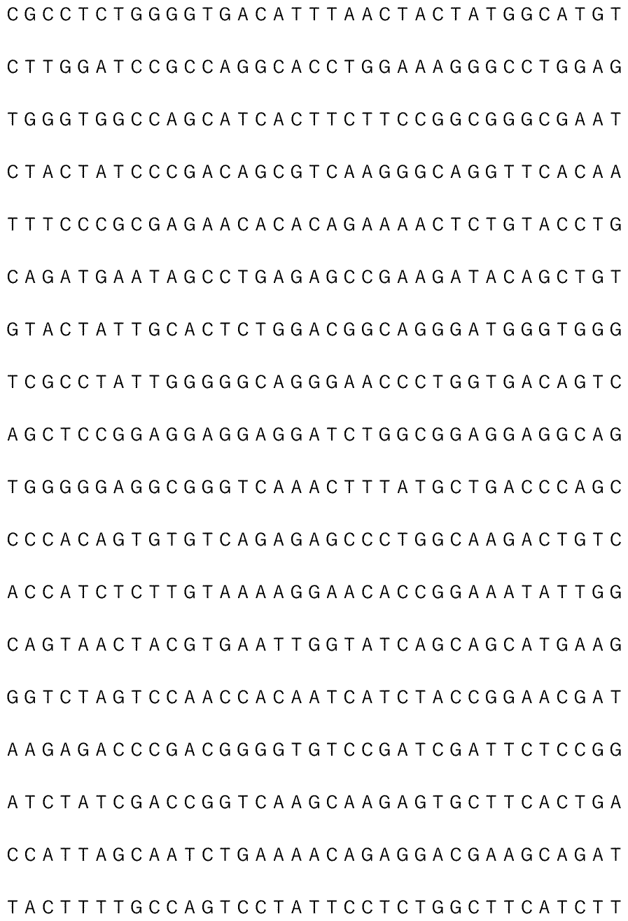

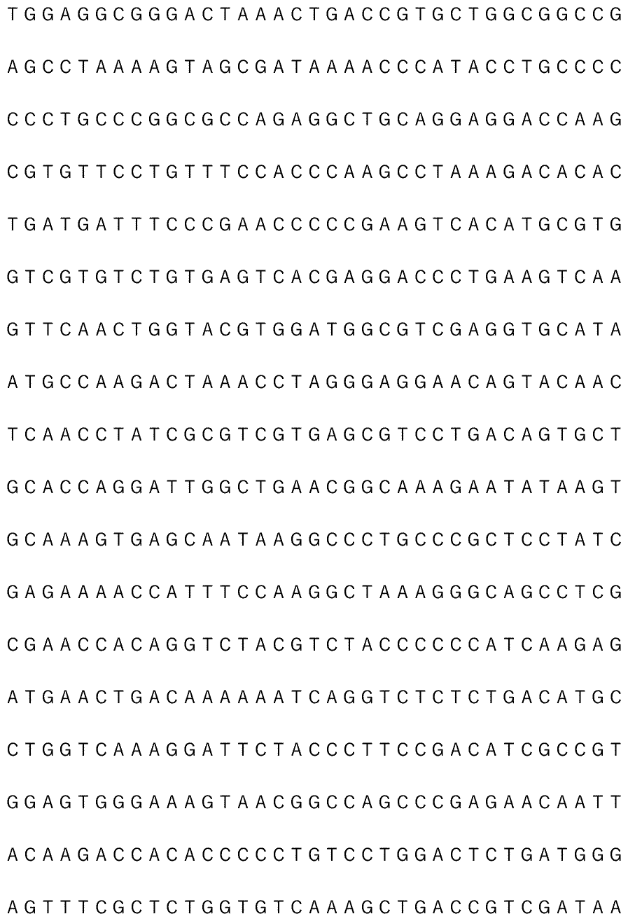

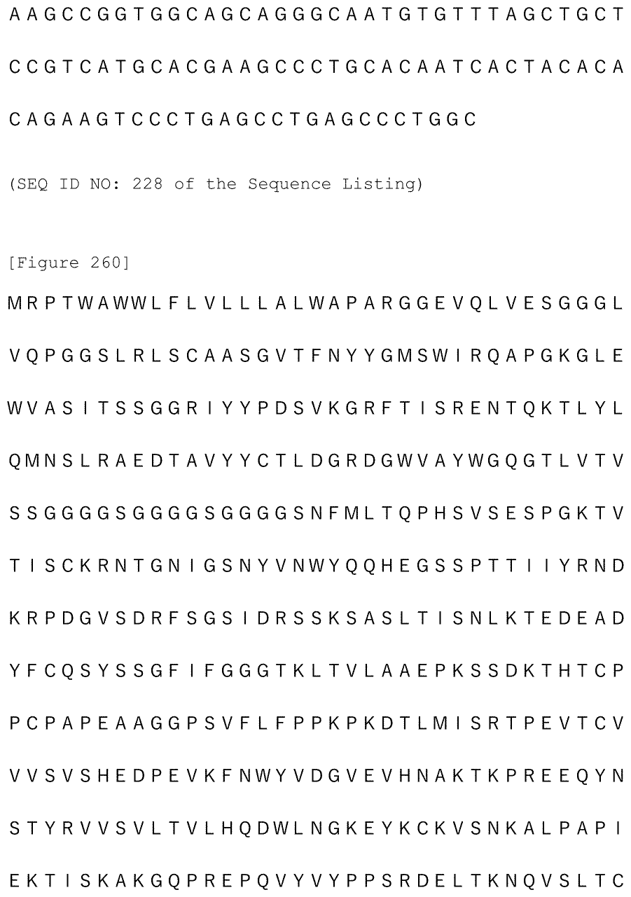

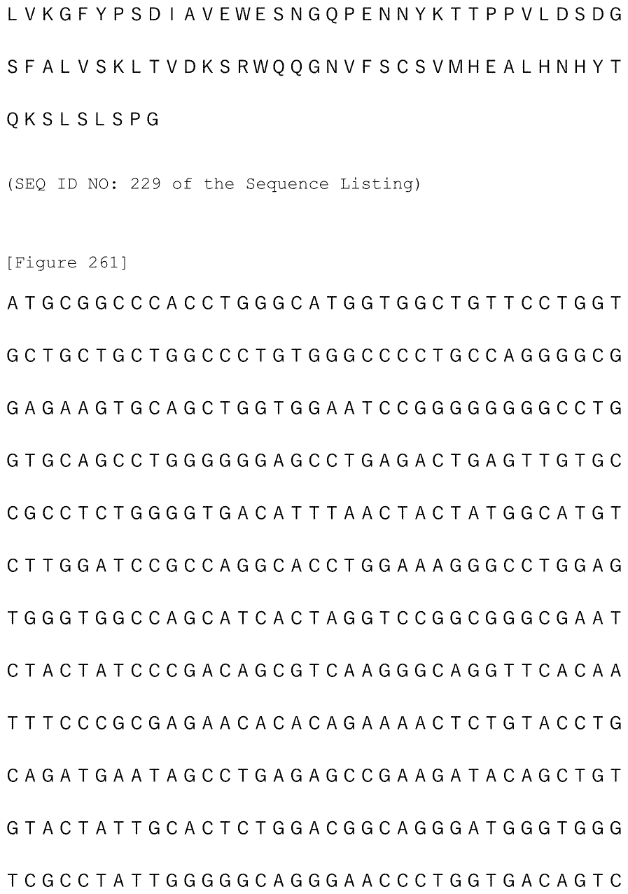

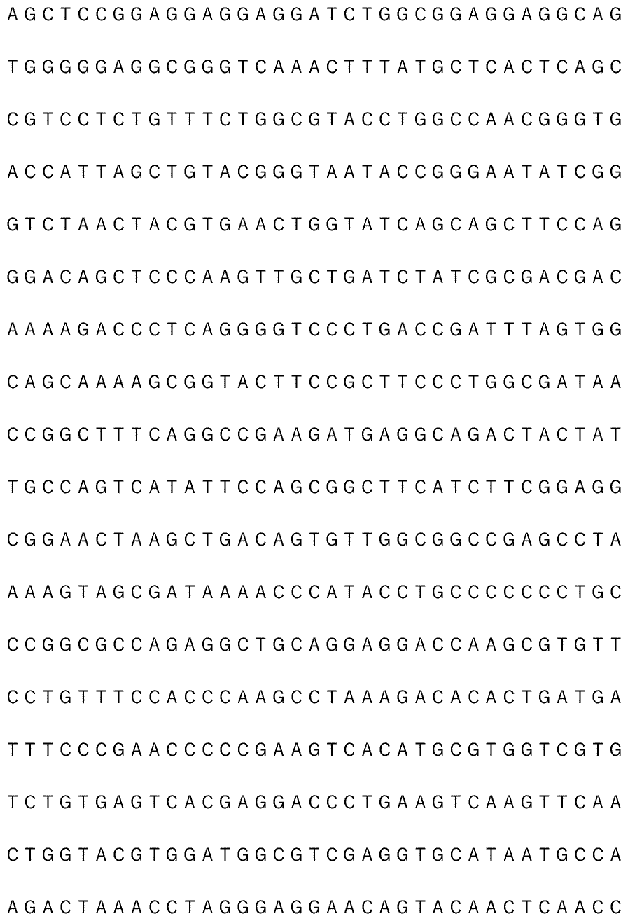

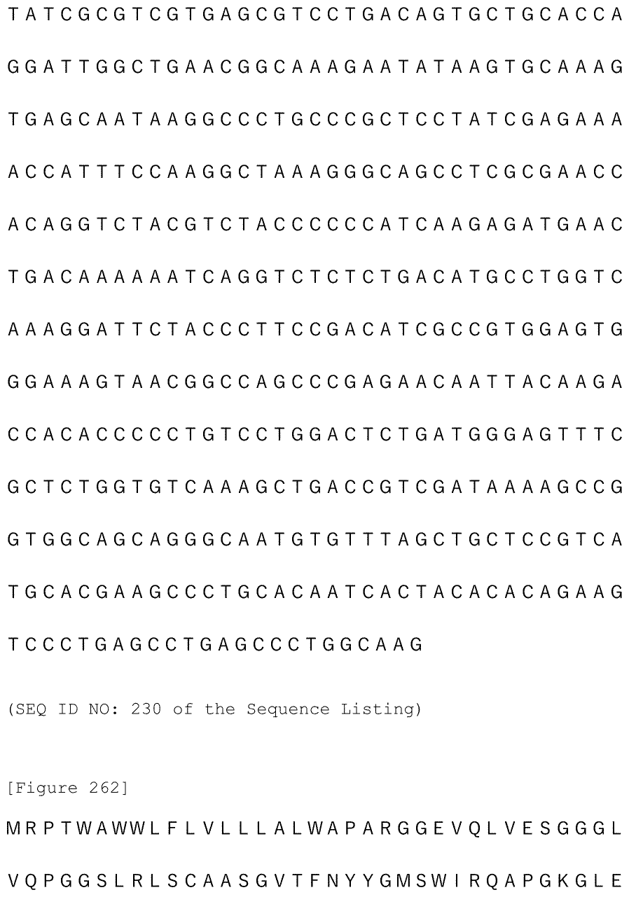

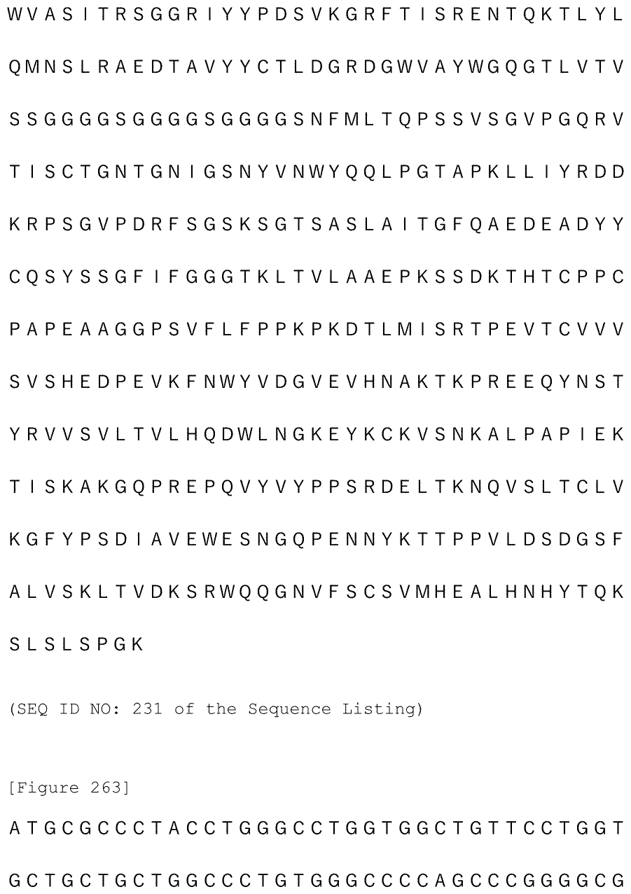

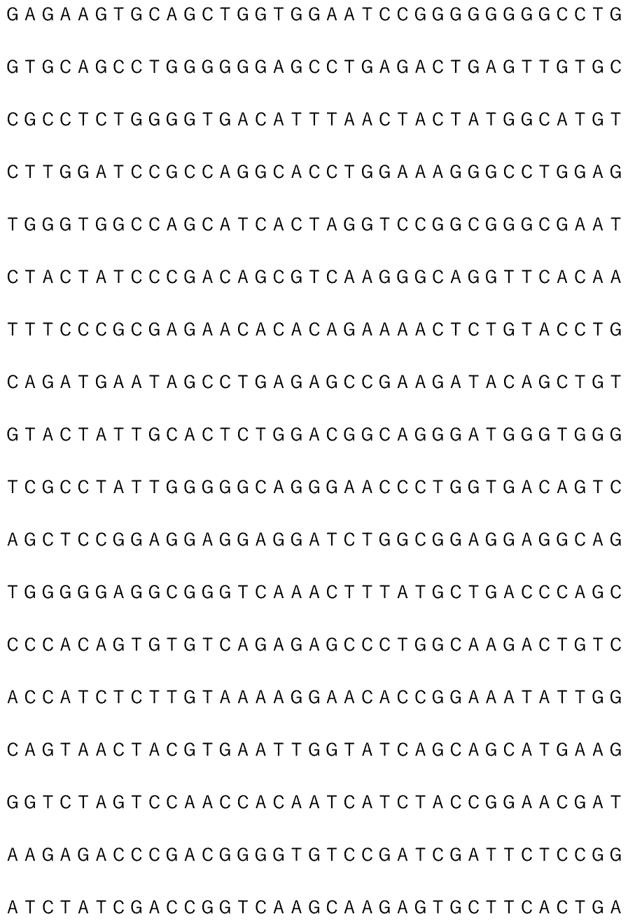

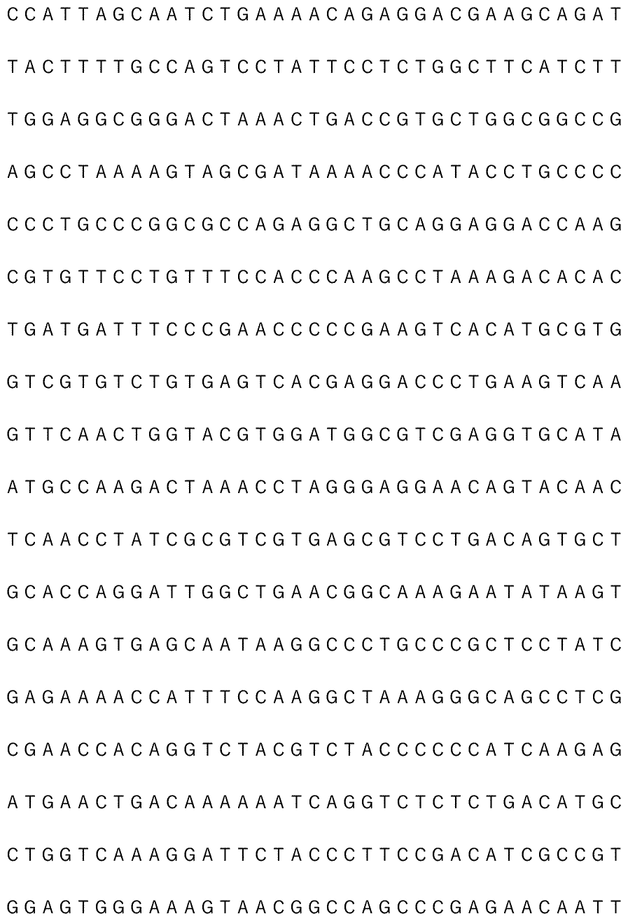

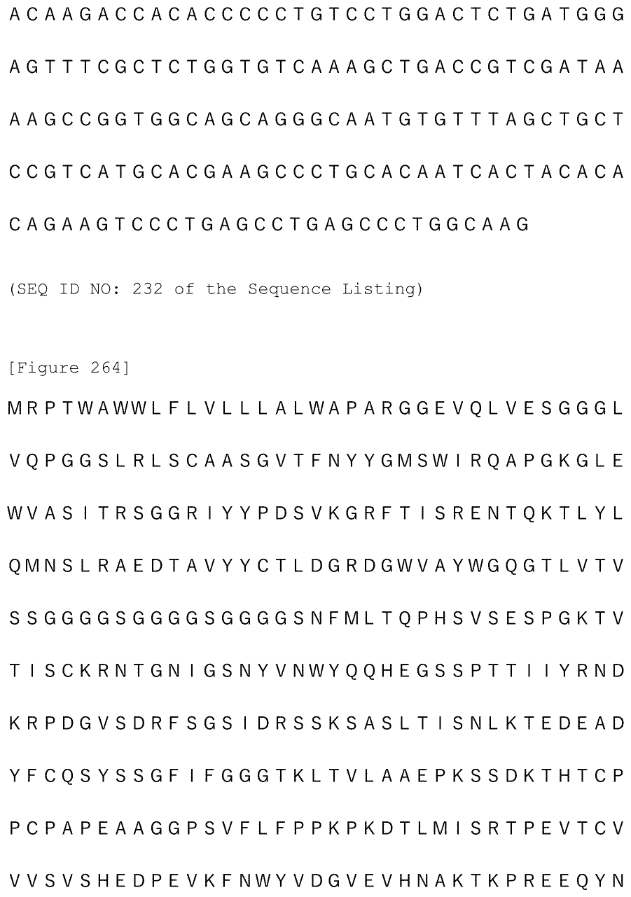

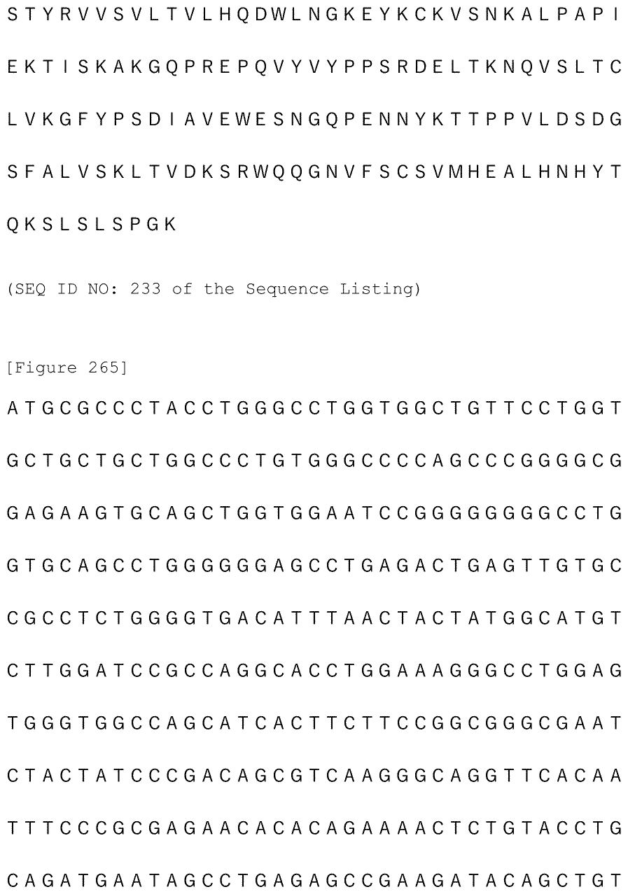

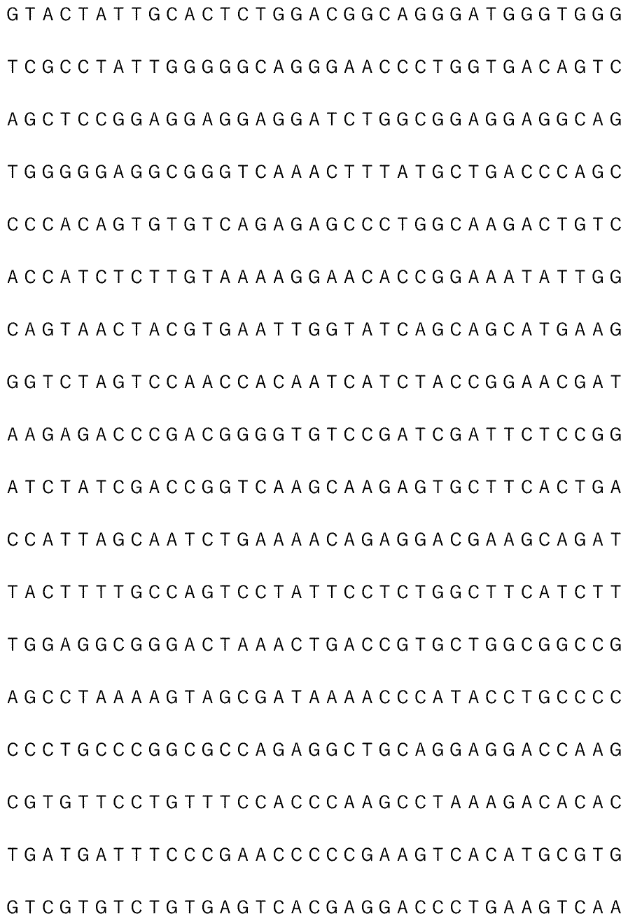

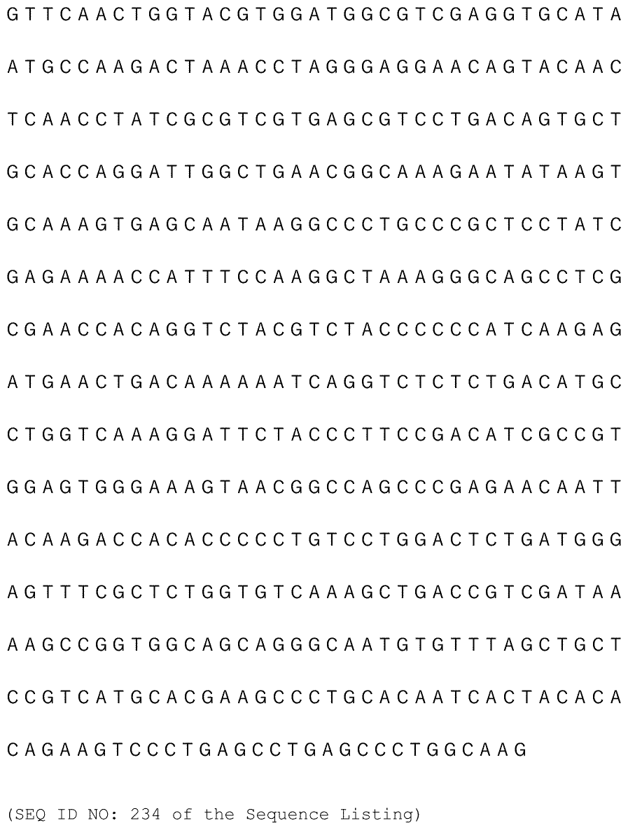

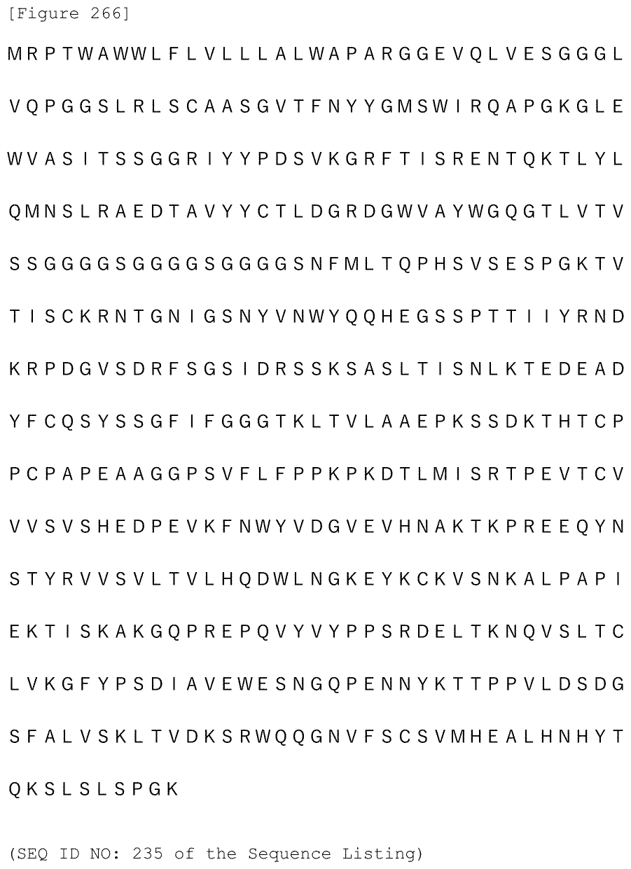

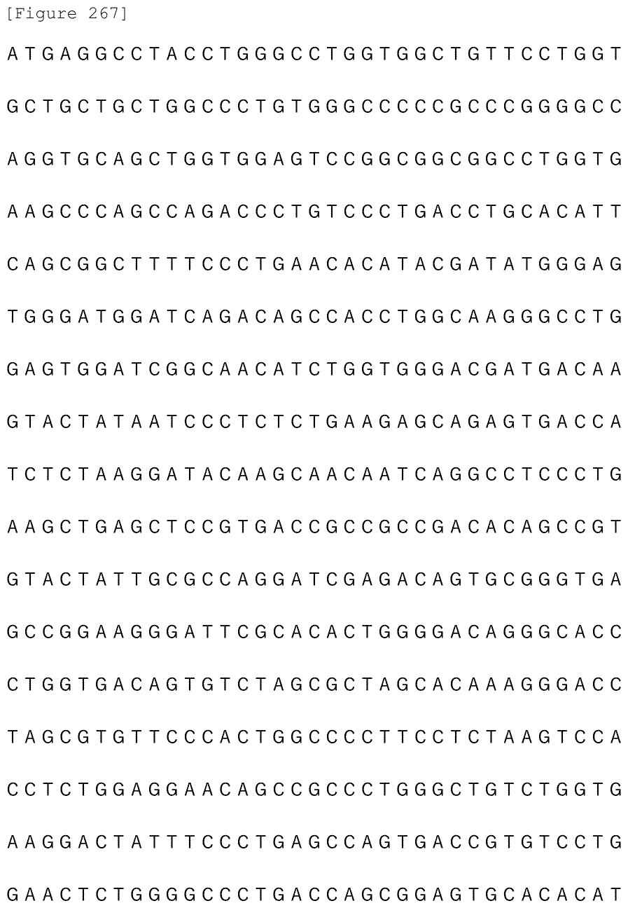

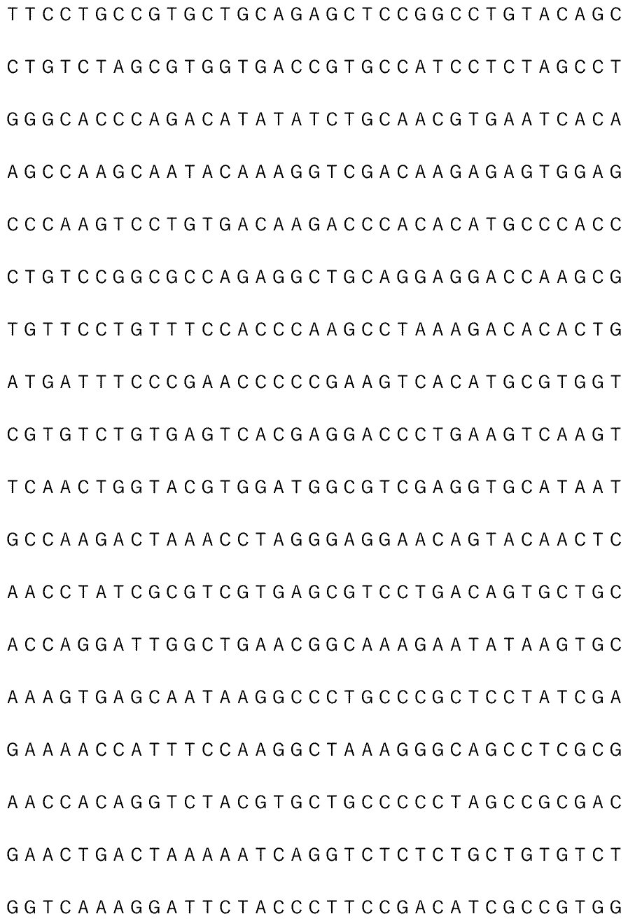

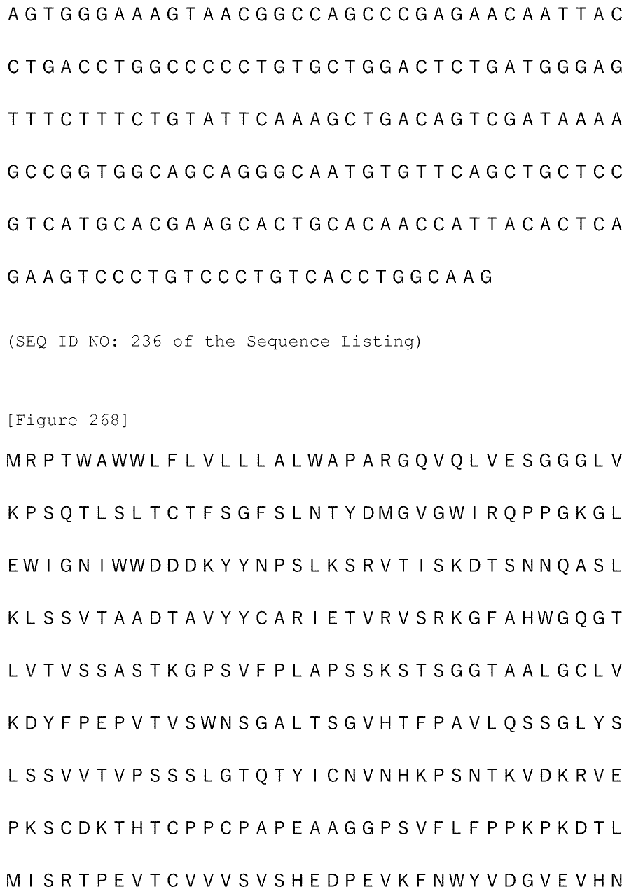

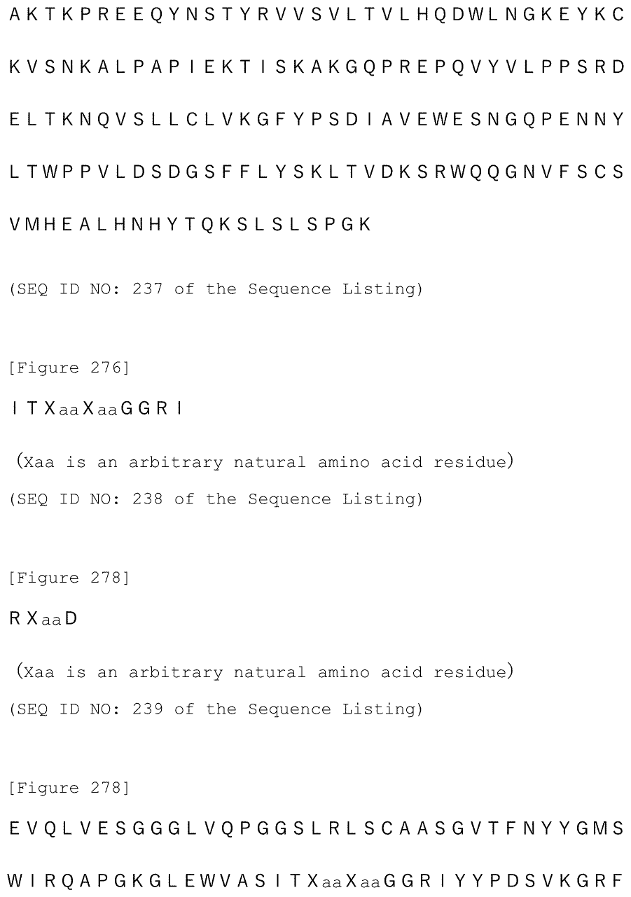

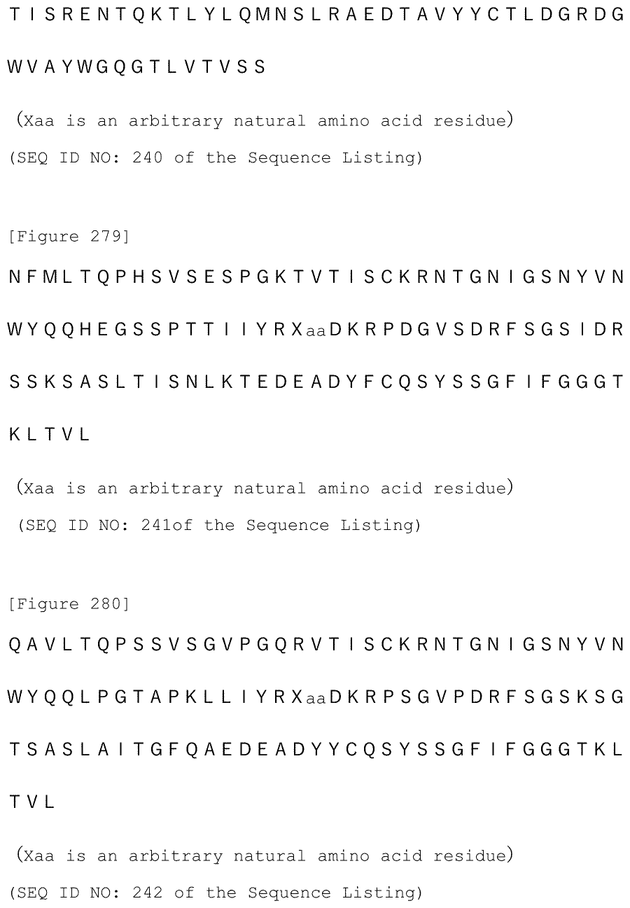

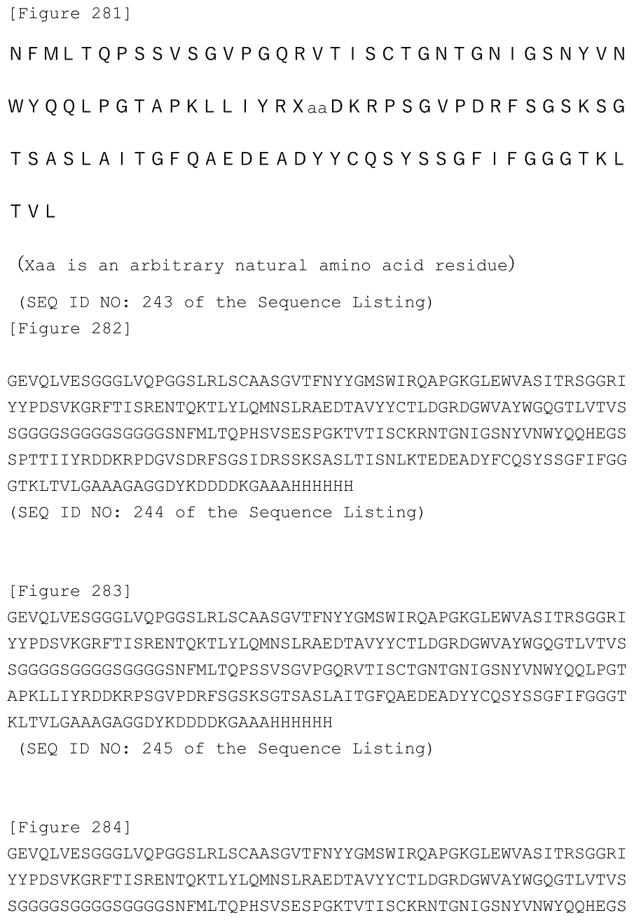

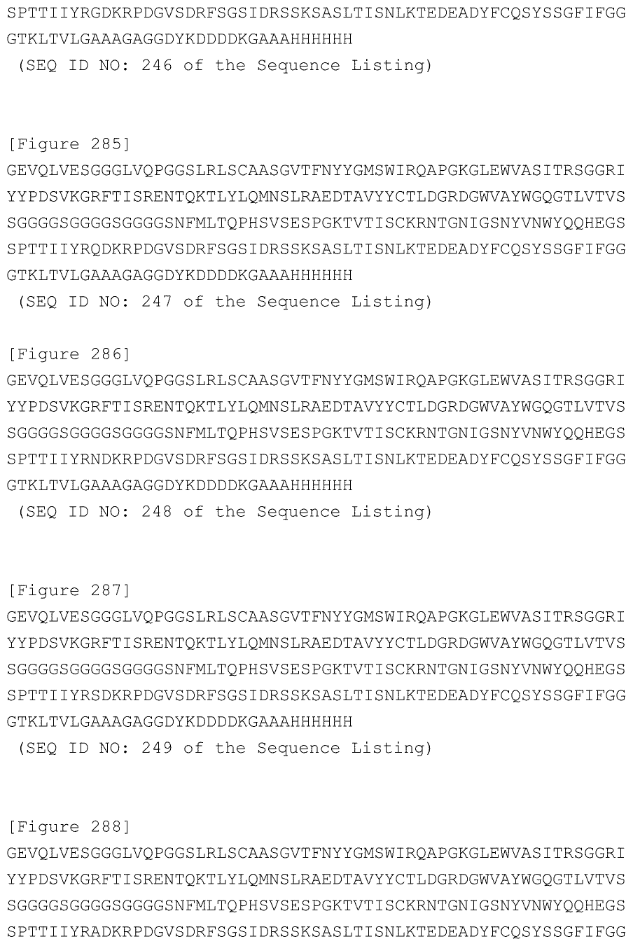

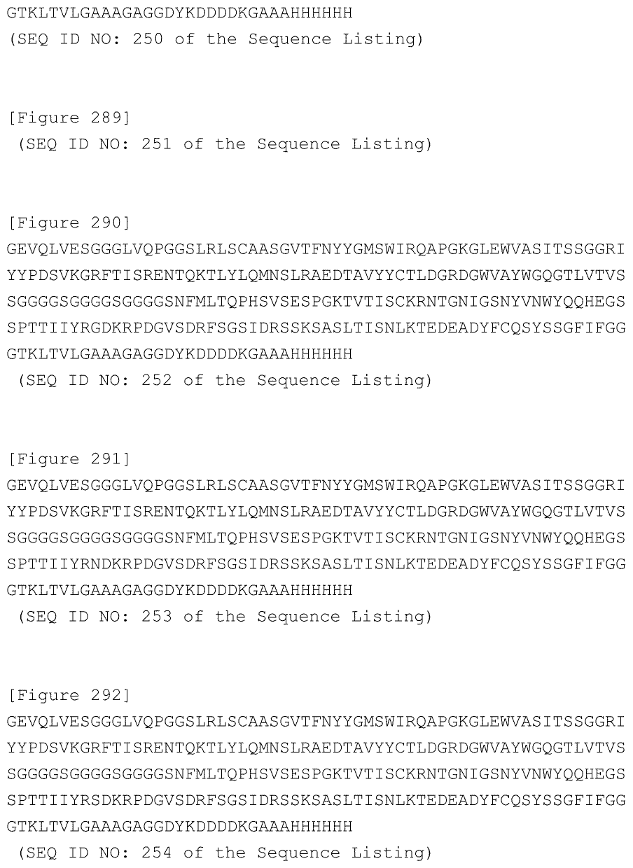

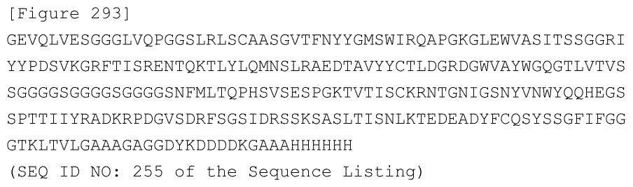

The amino acid sequences constituting v19126 obtained by expression from the respective vectors are shown in SEQ ID NOs: 219 (FIG. 243), 215 (FIG. 239), and 217 (FIG. 241) of the Sequence Listing. The amino acid sequences constituting v19125 are shown in SEQ ID NOs: 221 (FIG. 245), 215 (FIG. 239), and 217 (FIG. 241) of the Sequence Listing.

### 14)-2-3 Production of Dual-Type Bispecific Molecule

8000 μg of Polyethyleneimine max (PEImax, Polysciences) was dissolved in FreeStyle F17 medium to prepare a PEImax solution. 1000 μg of a mixture of the vectors pCL_#13557 and pCL_#13563 mixed at a ratio of 4:3, or the vectors pCL_#13561 and pCL_#13563 mixed at a ratio of 1:1, was added to an aliquot of F17 medium, and 1000 μg of a DNA mixture of pAKT and pGFP (both from National Research Council) mixed with already fragmented salmon sperm DNA (Sigma-Aldrich Corp.) was added to another aliquot of F17 medium. The PEImax solution, the vector mixture, and the pAKT/pGFP/salmon sperm DNA solution were combined, gently stirred, incubated for minutes, and then added to 2 L of CHO-3E7 cell suspension. The cells were shake-cultured at 37° C. for 1 day in a 5% CO2 incubator. 0.5 mM valproic acid (Sigma-Aldrich Corp.) and 0.1% (w/v) Tryptone N1 (Organotechnie) were then added. The cells were further shake-cultured at 32° C. for 6 days. On day 7 after the start of the culture, the culture supernatant was recovered and filtered through a 0.2 μm filter (Sartorius Japan K.K.) to prepare a sample for evaluation.

pCL_#13557 and pCL_#13563 were used in the expression and preparation of a dual-scFv (dual)-type bispecific molecule of C3E-7034 and h2B1 (v19122). pCL_#13561 and pCL_#13563 were used in the expression and preparation of a dual-type bispecific molecule of C3E-7036 and h2B1 (v19121).

The amino acid sequences constituting v19122 obtained by expression from the respective vectors are shown in SEQ ID NOs: 219 (FIG. 243) and 223 (FIG. 247) of the Sequence Listing. The amino acid sequences constituting v19121 are shown in SEQ ID NOs: 221 (FIG. 245) and 223 (FIG. 247) of the Sequence Listing.

### 14)-3 Purification of Fc-Containing Anti-GPRC5D-Anti-CD3 Bispecific Molecule

Each bispecific molecule was purified from the culture supernatant obtained in Example 14)-2 by two steps using protein A affinity chromatography and gel filtration chromatography.

The culture supernatant was applied to a MabSelect SuRe column (GE Healthcare Bio-Sciences Corp.) equilibrated with PBS (pH 7.4) to adsorb the bispecific molecule of interest thereon. Non-adsorbed components were removed with PBS. Then, the adsorbed component was eluted with an acetate buffer (pH 3.6). The eluted fractions were adjusted to neutral pH with a Tris buffer (pH 11), then concentrated, and applied to a gel filtration column (GE Healthcare Bio-Sciences Corp.) equilibrated in advance with PBS (pH 7.4). The peak fractions obtained by gel filtration chromatography were analyzed by SDS capillary electrophoresis (LabChip-Caliper) to recover fractions corresponding to the heterodimer of interest. The final recovered fractions were passed through a 0.2 micron filter to prepare a sterile purified sample. For the dual scFv-type bispecific molecule preparations, the recovered fractions were subsequently buffer-exchanged into HBsor (25 mM histidine, 5% sorbitol, pH 6.0)using G25 fine desalting resin in two tandem HiPrep 26/10 columns (GE Healthcare Bio-Sciences Corp.). The identity of the purified sample was confirmed by mass spectrometry and SDS-polyacrylamide electrophoresis (SDS-PAGE) to be the correctly-assembled anti-GPRC5D-anti-CD3 bispecific molecule of interest.

### Example 15

### In Vitro Activity Evaluation of Fc-Containing Anti-GPRC5D-Anti-CD3 Bispecific Molecule

### 15)-1 Evaluation of Binding Activity of Fc-Containing Anti-GPRC5D-Anti-CD3 Bispecific Molecule by Flow Cytometry

### 15)-1-1 Binding of Fc-Containing Anti-GPRC5D-Anti-CD3 Bispecific Molecule to Endogenous Human GPRC5D-Expressing Cells (KHM-1B)

A human multiple myeloma cell line KHM-1B expressing GPRC5D was adjusted to an appropriate concentration with PBS containing 5% FBS. LIVE/DEAD Fixable Near-IR Dead Cell Stain Kit was added to the cells, which were then left standing at 4° C. for 30 minutes. The cells were washed twice with PBS containing 5% FBS, then adjusted to a concentration of 1×10⁶ cells/mL with PBS containing 5% FBS, inoculated in an amount of 100 μL/well to a 96-well U-bottomed microplate, and centrifuged to remove a supernatant. Each Fc-containing anti-GPRC5D-anti-CD3 bispecific molecule (prepared in Example 14) diluted with PBS containing 5% FBS was added thereto in an amount of 100 μL/well, and the plate was left standing at 4° C. for 60 minutes. The cells were washed twice with PBS containing 5% FBS. Then, R-Phycoerythrin AffiniPure F(ab′)2 Fragment Goat Anti-Human IgG, Fcγ Fragment Specific (Jackson ImmunoResearch Laboratories, Inc.) diluted 100-fold with PBS containing 5% FBS was added thereto in an amount of 100 μL/well, and the plate was left standing at 4° C. for 1 hour. The cells were washed twice with PBS containing 5% FBS and then resuspended in PBS containing 5% FBS, followed by detection using a flow cytometer (FACSCanto™ II). The data was analyzed using Flowjo. The PE fluorescence intensity of the fraction free from dead cells was plotted to a histogram. The mean fluorescence intensity (MFI) was calculated. As a result, the Fc-containing anti-GPRC5D-anti-CD3 bispecific antibodies were found to bind to human GPRC5D-expressing cells (FIG. 248).

### 15)-1-2 Binding of Fc-Containing Anti-GPRC5D-Anti-CD3 Bispecific Molecule to Cynomolgus Monkey GPRC5D-Expressing Cells

Staining and analysis were carried out in the same way as in Example 15)-1-1 using the KMS-11_cGPRC5D cells prepared in Example 5)-2-2. As shown in FIG. 249, these Fc-containing anti-GPRC5D-anti-CD3 bispecific antibodies were found to bind to cynomolgus monkey GPRC5D-expressing cells.

### 15)-1-3 Binding of Fc-Containing Anti-GPRC5D-Anti-CD3 Bispecific Molecule to Human CD3 (Human PBMC)

Commercially available human PBMC (Cellular Technology Limited) was adjusted to an appropriate concentration with PBS containing 5% FBS. LIVE/DEAD Fixable Near-IR Dead Cell Stain Kit (Thermo Fisher Scientific Inc.) and an anti-CD19 antibody (Beckman Coulter Inc.) were added to the cells, which were then left standing at 4° C. for 30 minutes. The cells were washed twice with PBS containing 5% FBS, then adjusted to a concentration of 1×10⁶ cells/mL with PBS containing 5% FBS, inoculated in an amount of 100 μL/well to a 96-well U-bottomed microplate, and centrifuged to remove a supernatant. Each Fc-containing anti-GPRC5D-anti-CD3 bispecific molecule (prepared in Example 14) diluted with PBS containing 5% FBS was added thereto in an amount of 100 μL/well, and the plate was left standing at 4° C. for 60 minutes. The cells were washed twice with PBS containing 5% FBS. Then, R-Phycoerythrin AffiniPure F(ab′)2 Fragment Goat Anti-Human IgG, Fcγ Fragment Specific (Jackson ImmunoResearch Laboratories, Inc.) diluted 100-fold with PBS containing 5% FBS was added thereto in an amount of 100 μL/well, and the plate was left standing at 4° C. for 1 hour. The cells were washed twice with PBS containing 5% FBS and then resuspended in PBS containing 5% FBS, followed by detection using a flow cytometer (FACSCanto™ II). The data was analyzed using Flowjo. The PE fluorescence intensity of the fraction free from dead cells was plotted to a histogram. The mean fluorescence intensity (MFI) was calculated. As a result, the Fc-containing anti-GPRC5D-anti-CD3 bispecific antibodies were found to bind to human CD3-expressing cells (FIG. 250).

### 15)-1-4 Binding of Fc-Containing Anti-GPRC5D-Anti-CD3 Bispecific Molecule to Cynomolgus Monkey CD3 (Cynomolgus Monkey PBMC)

Staining and analysis were carried out in the same way as in Example 15)-1-3 using the cynomolgus monkey PBMC collected in the same way as in Example 13)-1-4. As shown in FIG. 251, these Fc-containing anti-GPRC5D-anti-CD3 bispecific antibodies were found to bind to cynomolgus monkey CD3-expressing cells.

### 15)-2 Cytotoxic Activity Evaluation of Fc-Containing Anti-GPRC5D-Anti-CD3 Bispecific Molecule

### 15)-2-1 Preparation of Target Cell

KHM-1B cells were prepared in the same way as in Example 13)-2-1 and used as target cells.

### 15)-2-2 Preparation of Effector Cell

Commercially available frozen PBMC (Cellular Technology Limited) was thawed at 37° C., transferred to a solution of an RPMI1640 medium containing 10% FBS supplemented with Anti-aggregate Wash reagent (Cellular Technology Limited), washed twice, then adjusted to 1.5×105 cells/mL with an RPMI1640 medium containing 10% FBS, and used as effector cells.

### 15)-2-3 Cytotoxicity Assay

The KHM-1B cells obtained in Example 15)-2-1 were added at a concentration of 50 μL/well to a 96-well U-bottomed microplate. Each Fc-containing anti-GPRC5D-anti-CD3 bispecific molecule (prepared in Example 14) adjusted to varying concentrations was added thereto in an amount of 50 μL/well. The effector cells prepared in Example 15)-2-2 were added thereto in an amount of 100 μL/well. After centrifugation at room temperature at 1000 rpm for 1 minute, the cells were cultured at 37° C. for 24 or 48 hours under 5% CO₂ conditions. A 50 μL aliquot of the supernatant was recovered into LumaPlate (PerkinElmer, Inc.) and dried at 50° C. for approximately 2 hours, followed by measurement using a plate reader (TopCount; PerkinElmer, Inc.). The percentage of cells lysed was calculated according to the following expression:

Percentage of cells lysed (%)=(A−B)/(C−B)×100
A: Count of sample well
B: Average of background (antibody-unsupplemented wells) counts (n=3). 50 μL of a medium for assay was added instead of adding the antibody. The other procedures were the same as in the case of the sample well.
C: Average of maximum release (wells containing target cells lysed in a surfactant) counts (n=3). 50 μL of a medium for assay was added instead of adding the antibody. 100 μL of the surfactant was added, and the 50 μL aliquot was transferred to LumaPlate, as with the sample well, and assayed.

As shown in FIG. 252, these Fc-containing anti-GPRC5D-anti-CD3 bispecific antibodies exhibited cytotoxic activity against the KHM-1B cells.

### Example 16

### In Vivo Activity Evaluation of Fc-Containing Anti-GPRC5D-Anti-CD3 Bispecific Molecule

### 16)-1 In Vivo Activity in Tumor/PBMC Co-Grafting Model

A human multiple myeloma cell line KHM-1B (JCRB) and human PBMC (Cellular Technology Limited) were each adjusted to 5×10⁷ cells/mL with PBS containing 50% Matrigel (Corning Inc.) and subcutaneously co-grafted in an amount of 0.1 mL to each NOD-Scid mouse (female, 5 weeks old). After the inoculation, the mice were grouped, and each anti-GPRC5D-anti-CD3 bispecific molecule was administered (0.1 mg/kg) into the tail veins. The administration was carried out three times every day from the inoculation day (day 0) to day 2. The major axis (mm) and minor axis (mm) of the tumor were measured over time from 1 week later (day 7) using an electronic digital caliper. The estimated tumor volume was calculated according to the following expression:

Estimated tumor volume (mm³)=Average estimated tumor volume of the individuals

Estimated tumor volume of each individual=Major axis×Minor axis²/2

An anti-tumor efficacy was confirmed in each anti-GPRC5D-anti-CD3 bispecific molecule administration group (FIG. 253).

### 16)-2 In Vivo Activity in Established Tumor Model in Human PBMC Reconstituted Mice

Human PBMC was adjusted to 5×10⁷ cells/mL with PBS and implanted in an amount of 0.2 mL into the tail vein of each NOG mouse (female, 6 weeks old) (day −4). On day 0, KHM-1B was adjusted to 3×10⁷ cells/mL with PBS containing 50% Matrigel and subcutaneously inoculated in an amount of 0.1 mL to the NOG mouse. When the estimated tumor volume of the mouse reached approximately 200 mm³ (day 12), the mice were grouped according to their tumor volumes, and each anti-GPRC5D-anti-CD3 bispecific molecule was administered (1 mg/kg) into the tail veins. The administration was carried out on days 12, 15, and 18. The major axis (mm) and minor axis (mm) of the tumor were measured over time using an electronic digital caliper. The estimated tumor volume was calculated. The anti-GPRC5D-anti-CD3 bispecific antibodies exhibited tumor regression. Particularly, a strong tumor regression efficacy was confirmed in the v19125 treatment group (FIG. 254).

### Example 17

### Preparation of CDR-Modified Hybrid-Type Anti-GPRC5D-Anti-CD3 Bispecific Molecule

### 17)-1 Preparation of CDR-Modified Hybrid-Type Anti-GPRC5D-Anti-CD3 Bispecific Molecule Expression Vector

### 17)-1-1 Preparation of CDR-Modified Hybrid-Type Anti-GPRC5D-Anti-CD3 Bispecific Molecule (C5D-0004, C5D-0005, and C5D-0006) Expression Vectors

Among the hybrid-type bispecific molecule (v19125) expression vectors constructed in Example 14)-1-2, pCL_#13561 encoding the humanized anti-CD3 scFv-Fc was used as a template in site-directed mutagenesis to prepare a vector pC3E-8015 for a CDR-modified form containing Arg in place of Asn53 of H chain CDR2. Likewise, among the hybrid-type bispecific molecule (v19126) expression vectors, pCL_#13557 encoding the humanized anti-CD3 scFv-Fc was used as a template in site-directed mutagenesis to prepare a vector pC3E-8017 for a CDR-modified form containing Arg in place of Asn53 of H chain CDR2 and Asn in place of L chain Asp52. Also, pCL_#13557 was used as a template in site-directed mutagenesis to prepare a vector pC3E-8018 for a CDR-modified form containing Ser in place of Asn53 of H chain CDR2 and Asn in place of L chain Asp52.

The ORF sequences of pC3E-8015, pC3E-8017, and pC3E-8018 are shown in SEQ ID NO: 224 (FIG. 255), SEQ ID NO: 226 (FIG. 257), and SEQ ID NO: 228 (FIG. 259), respectively, of the Sequence Listing.

### 17)-1-2 Preparation of C-Terminally Lys-Added CDR-Modified Hybrid-Type Anti-GPRC5D-Anti-CD3 Bispecific Molecule (C5D-0014, C5D-0015, and C5D-0016) Expression Vectors

Among the hybrid-type bispecific molecule (C5D-0004) expression vectors constructed in Example 17)-1-1, pC3E-8015 encoding the CDR-modified humanized anti-CD3 scFv-Fc was used as a template in site-directed mutagenesis to prepare a vector pC3E-8025 for a K-added CDR-modified form containing Lys inserted to the C terminus of Fc. Likewise, pC3E-8017 or pC3E-8018 was used as a template in a site-directed mutagenesis to prepare vectors pC3E-8027 and pC3E-8028 for K-added CDR-modified forms containing Lys inserted in the C terminus of Fc.

Among the hybrid-type bispecific molecule (v19125 and v19126) expression vectors constructed in Example 14)-1-2, pCL_#13555 encoding the anti-GPRC5D Fab-Fc was used as a template in site-directed mutagenesis to prepare a vector pTAA_#2 for a K-added form containing Lys inserted in the C terminus of Fc.

The ORF sequences of pC3E-8025, pC3E-8027, pC3E-8028, and pTAA_#2 are shown in SEQ ID NO: 230 (FIG. 261), SEQ ID NO: 232 (FIG. 263), SEQ ID NO: 234 (FIG. 265), and SEQ ID NO: 236 (FIG. 267), respectively, of the Sequence Listing.

### 17)-2 Production of CDR-Modified Hybrid-Type Anti-GPRC5D-Anti-CD3 Bispecific Molecule

CHO-3E7 cells were subcultured and cultured according to the supplier's manual (National Research Council Canada, Raymond C. et al., Methods (2011) 55 (1), 44-51). The solution of the CHO-3E7 cells in the logarithmic growth phase was diluted to 2×10⁶10\hat{ }6 cells/mL with BalanCD Transfectory CHO (Irvine Scientific) containing 4 mM glutamine and used in the production of various bispecific antibodies.

ExpiCHO-S cells were subcultured and cultured according to the supplier's manual (Thermo Fisher Scientific Inc.). The solution of the ExpiCHO-S cells in the logarithmic growth phase was diluted to 6×10⁶ cells/mL with ExpiCHO Expression Medium (Thermo Fisher Scientific Inc.) and used in the production of various bispecific antibodies.

### 17)-2-1 Production of CDR-Modified Hybrid-Type Anti-GPRC5D-anti-CD3 Bispecific Antibodies (C5D-0004, C5D-0005, and C5D-0006)

The hybrid-type anti-GPRC5D-anti-CD3 bispecific antibodies C5D-0004, C5D-0005, and C5D-0006 were expressed by culture using the ExpiCHO-S cells as a host. A method for transfecting the cells with the expression vectors and culture conditions were all carried out according to the manual attached to the product (Thermo Fisher Scientific Inc.). The culture was performed on a scale of 750 mL, and conditions of the Max titer protocol described in the manual were used for feed addition and a culture temperature. On 13 days after the start of culture, the culture supernatant was recovered and filtered through a 0.2 μm filter (Sartorius Japan K.K.) to prepare a sample for evaluation.

A hybrid-type bispecific molecule C5D-0004 was obtained from the combination of pC3E-8015, pCL_#13555, and pCL_#12123. A hybrid-type bispecific molecule C5D-0005 was obtained from the combination of pC3E-8017, pCL_#13555, and pCL_#12123. A hybrid-type bispecific molecule C5D-0006 was obtained from the combination of pC3E-8018, pCL_#13555, and pCL_#12123.

The amino acid sequences constituting C5D-0004 obtained by expression from the respective vectors are shown in SEQ ID NOs: 225 (FIG. 256), 215 (FIG. 239), and 217 (FIG. 241) of the Sequence Listing. The amino acid sequences constituting C5D-0005 are shown in SEQ ID NOs: 227 (FIG. 258), 215 (FIG. 239), and 217 (FIG. 241) of the Sequence Listing. The amino acid sequences constituting C5D-0006 are shown in SEQ ID NOs: 229 (FIG. 260), 215 (FIG. 239), and 217 (FIG. 241) of the Sequence Listing.

### 17)-2-2 Production of C-Terminally Lys-Added CDR-Modified Hybrid-Type Anti-GPRC5D-Anti-CD3 Bispecific Antibodies (C5D-0014, C5D-0015, and C5D-0016)

800 μg of PEImax (Polysciences) was dissolved in 3 mL of an Opti-PRO SFM medium (Thermo Fisher Scientific Inc.) to prepare a PEImax solution. 100 μg of a vector mixture of pC3E-_8025, pTAA_#_#2, and pCL_#_#12123 mixed at a ratio of 1:1:1.5 was added to 3 mL of an Opti-PRO SFM medium, and 100 μg of a DNA mixture of pAKT and pGFP mixed with already fragmented salmon sperm DNA was added to 3 mL of an Opti-PRO SFM medium. The PEImax solution, the vector mixture, and the DNA solution were combined, gently stirred, left for 5 minutes, and then added to 200 mL of the CHO-3E7 cell solution. The cells were shake-cultured at 37° C. for 1 day in a 5% CO2 incubator. Then, 22 mL of Transfectory Supplement (Irvine Scientific), 480 μL of Anti clumping supplement (Thermo Fisher Scientific Inc.), and 500 μLuL of valproic acid (Sigma-Aldrich Corp.) were added thereto. The cells were further shake-cultured at 32° C. for 9 days. On 10 days after the start of the culture, the culture supernatant was recovered and filtered through a 0.2 μm filter (Sartorius Japan K.K.) to prepare a sample for evaluation.

A hybrid-type bispecific molecule C5D-0014 was obtained from the combination of pC3E-8025, pTAA_#_#2, and pCL_#_12123. A hybrid-type bispecific molecule C5D-0015 was obtained from the combination of pC3E-8027, pTAA_#_#2, and pCL_#_#12123. A hybrid-type bispecific molecule C5D-0016 was obtained from combination of pC3E-8028, pTAA_#_#2, and pCL_#_#12123.

The amino acid sequences constituting C5D-0014 obtained by expression from the respective vectors are shown in SEQ ID NOs: 231 (FIG. 262), 237 (FIG. 268), and 217 (FIG. 241) of the Sequence Listing. The amino acid sequences constituting C5D-0015 are shown in SEQ ID NOs: 233 (FIG. 264), 237 (FIG. 268), and 217 (FIG. 241) of the Sequence Listing. The amino acid sequences constituting C5D-0016 are shown in SEQ ID NOs: 235 (FIG. 266), 237 (FIG. 268), and 217 (FIG. 241) of the Sequence Listing.

### 17)-3 Purification of CDR-Modified Hybrid-Type Anti-GPRC5D-Anti-CD3 Bispecific Antibodies

### 17)-3-1 Purification of CDR-Modified Hybrid-Type Anti-GPRC5D-Anti-CD3 Bispecific Antibodies (C5D-0004, C5D-0005, and C5D-0006)

Each bispecific molecule was purified from the culture supernatant obtained in Example 17)-2-1 by three steps using protein A affinity chromatography, hydroxyapatite chromatography, and cation-exchange chromatography. In brief, the culture supernatant was applied to a MabSelect SuRe column (GE Healthcare Bio-Sciences Corp.) equilibrated with PBS (pH 7.4) to adsorb the bispecific molecule of interest thereon. Non-adsorbed components were removed with PBS. Then, the adsorbed component was eluted with a 100 mM acetate buffer (pH 3.5). The eluted fractions were immediately adjusted to neutral pH with a Tris buffer (pH 9.0), then dialyzed against 50 mM HEPES, 10 mM potassium phosphate, and a 100 mM sodium chloride solution, and applied to a hydroxyapatite column Bio-Scale CHT Type-I (Bio-Rad Laboratories, Inc.). The adsorbed bispecific molecule of interest was eluted by changing the sodium chloride concentration in the solvent from 0.1 M to 1 M by the linear concentration gradient. The obtained peak fractions were analyzed by SDS-PAGE to recover fractions corresponding to the bispecific molecule of interest. Next, the recovered fractions were buffer-replaced with 50 mM HEPES (pH 8.0) and a 20 mM sodium chloride solution and then applied to a cation-exchange column Mono S (GE Healthcare Bio-Sciences Corp.). The adsorbed bispecific molecule of interest was eluted by changing the sodium chloride concentration in the solvent from 20 mM to 1 M by the linear concentration gradient. The obtained peak fractions were analyzed by SDS-PAGE to recover fractions corresponding to the bispecific molecule of interest. The finally recovered fractions were dialyzed against HBsor (25 mM histidine, 5% sorbitol, pH 6.0) and filtered through a filter to prepare a purified sample. The purified sample was definitely confirmed by mass spectrometry, SDS-PAGE, and SEC analysis to be the anti-GPRC5D-anti-CD3 bispecific molecule of interest.

### 17)-3-2 Purification of C-Terminally Lys-Added CDR-Modified Hybrid-Type Anti-GPRC5D-Anti-CD3 Bispecific Antibodies (C5D-0014, C5D-0015, and C5D-0016)

Each bispecific molecule was purified from the culture supernatant obtained in Example 17)-2-2 by two steps using protein A affinity chromatography and hydroxyapatite chromatography. In brief, the culture supernatant was applied to a MabSelect SuRe column (GE Healthcare Bio-Sciences Corp.) equilibrated with PBS (pH 7.4) to adsorb the bispecific molecule of interest thereon. Non-adsorbed components were removed with PBS. Then, the adsorbed component was eluted with a 100 mM acetate buffer (pH 3.0). The eluted fractions were immediately adjusted to neutral pH with a Tris buffer (pH 9.5), then dialyzed against 50 mM HEPES, 10 mM potassium phosphate, and a 100 mM sodium chloride solution, and applied to a hydroxyapatite column Bio-Scale CHT Type-I (Bio-Rad Laboratories, Inc.). The adsorbed bispecific molecule of interest was eluted by changing the sodium chloride concentration in the solvent from 0.1 M to 1 M by the linear concentration gradient. The obtained peak fractions were analyzed by SDS-PAGE to recover fractions corresponding to the bispecific molecule of interest. The finally recovered fractions were dialyzed against HBsor (25 mM histidine, 5% sorbitol, pH 6.0) and filtered through a filter to prepare a purified sample. The purified sample was definitely confirmed by mass spectrometry, SDS-PAGE, and SEC analysis to be the anti-GPRC5D-anti-CD3 bispecific molecule of interest.

### Example 18

### In Vitro Activity Evaluation of C-Terminally Lys-Added CDR-Modified Hybrid-Type and CDR-Modified Hybrid-Type Anti-GPRC5D-Anti-CD3 Bispecific Antibodies

### 18)-1 Binding Activity Evaluation of C-Terminally Lys-Added CDR-Modified Hybrid-Type and CDR-Modified Hybrid-Type Anti-GPRC5D-Anti-CD3 Bispecific Antibodies by Flow Cytometry

### 18)-1-1 Binding of C-Terminally Lys-Added CDR-Modified Hybrid-type and CDR-Modified Hybrid-Type Anti-GPRC5D-Anti-CD3 Bispecific Antibodies to Endogenous Human GPRC5D-Expressing Cells (KHM-1B)

Cell preparation, staining, and analysis were carried out in the same way as in Example 15)-1-1. As a result, the anti-GPRC5D-anti-CD3 bispecific antibodies were found to bind to human GPRC5D-expressing cells (FIG. 269).

### 18)-1-2-2 Binding of C-Terminally Lys-Added CDR-Modified Hybrid-Type and CDR-Modified Hybrid-Type Anti-GPRC5D-Anti-CD3 Bispecific Antibodies to Cynomolgus Monkey GPRC5D-Expressing Cells

Cell preparation, staining, and analysis were carried out in the same way as in Example 15)-1-2. As shown in FIG. 270, these anti-GPRC5D-anti-CD3 bispecific antibodies were found to bind to cynomolgus monkey GPRC5D-expressing cells.

### 18)-1-3 Binding of C-Terminally Lys-Added CDR-Modified Hybrid-Type and CDR-Modified Hybrid-Type Anti-GPRC5D-Anti-CD3 Bispecific Antibodies to Human CD3 (Human PBMC)

Cell preparation, staining, and analysis were carried out in the same way as in Example 15)-1-3. As a result, the anti-GPRC5D-anti-CD3 bispecific antibodies were found to bind to human CD3-expressing cells (FIG. 271).

### 18)-1-4 Binding of C-Terminally Lys-Added CDR-Modified Hybrid-Type and CDR-Modified Hybrid-Type Anti-GPRC5D-Anti-CD3 Bispecific Antibodies to Cynomolgus Monkey CD3 (Cynomolgus Monkey PBMC)

Cell preparation, staining, and analysis were carried out in the same way as in Example 15)-1-4. As shown in FIG. 272, these anti-GPRC5D-anti-CD3 bispecific antibodies were found to bind to cynomolgus monkey CD3-expressing cells.

### 18)-2 Cytotoxic Activity Evaluation of C-Terminally Lys-Added CDR-Modified Hybrid-Type and CDR-Modified Hybrid-Type Anti-GPRC5D-Anti-CD3 Bispecific Antibodies

The cytotoxic activity assay was carried out in the same way as in Example 15)-2-3 except that the culture time was 24, 48 or 72 hours. As shown in FIG. 273, these anti-GPRC5D-anti-CD3 bispecific antibodies exhibited cytotoxic activity against KHM-1B cells.

### Example 19

### In Vivo Activity Evaluation of CDR-Modified Hybrid-Type, and C-Terminally Lys-Added CDR-Modified Hybrid-Type Anti-GPRC5D-Anti-CD3 Bispecific Molecule

### 19)-1 In Vivo Activity in Tumor/PBMC Co-Grafting Model

KHM-1B and human PBMC were each adjusted to 5×10⁷ cells/mL with PBS containing 50% Matrigel and subcutaneously cotransplanted in each amount of 0.1 mL to each NOD-Scid mouse (female, 5 weeks old). After the inoculation, the mice were grouped, and each anti-GPRC5D-anti-CD3 bispecific molecule was administered (1 μg/kg) into the tail veins. The administration was carried out three times every day from the inoculation day (day 0) to day 2. The major axis (mm) and minor axis (mm) of the tumor were measured over time from 1 week later (day 7) using an electronic digital caliper. The estimated tumor volume was calculated according to the following expression:

Estimated tumor volume (mm³)=Average estimated tumor volume of the individuals

Estimated tumor volume of each individual=Major axis×Minor axis²/2

An anti-tumor efficacy was confirmed in each anti-GPRC5D-anti-CD3 bispecific molecule administration group (FIG. 274).

### 19)-2 In Vivo Activity in Established Tumor Model in Human PBMC Reconstituted Mice

Human PBMC was adjusted to 5×10⁷ cells/mL with PBS and implanted in an amount of 0.2 mL into the tail vein of each NOG mouse (female, 6 weeks old) (day −4). On day 0, KHM-1B was adjusted to 3×10⁷ cells/mL with PBS containing 50% Matrigel and subcutaneously inoculated in an amount of 0.1 mL to the NOG mouse. When the estimated tumor volume of the mouse reached approximately 200 mm³ (day 11), the mice were grouped according to their tumor volumes, and the each anti-GPRC5D-anti-CD3 bispecific molecule was administered (1 mg/kg) into the tail veins. The administration was carried out on days 11, 14, 15, and 17. The major axis (mm) and minor axis (mm) of the tumor were measured over time using an electronic digital caliper. The estimated tumor volume was calculated. The anti-GPRC5D-anti-CD3 bispecific antibodies exhibited tumor regression (C5D-0004 in FIG. 275A, C5D-0014 in FIG. 275B).

### [Sequence Listing Free Text]

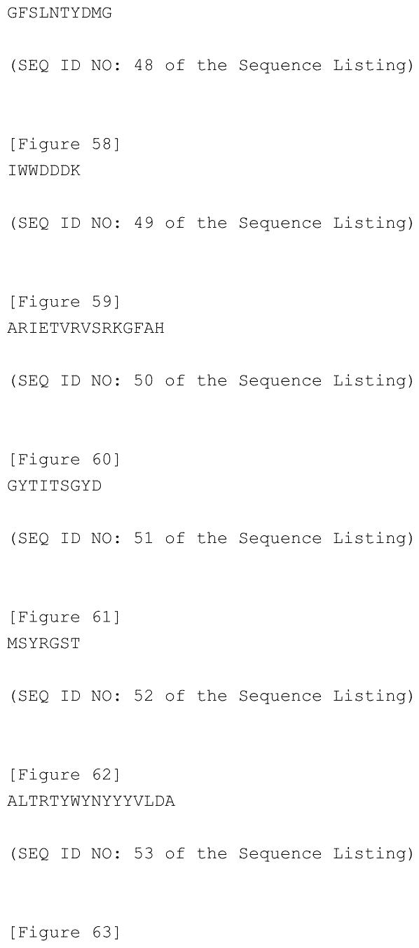

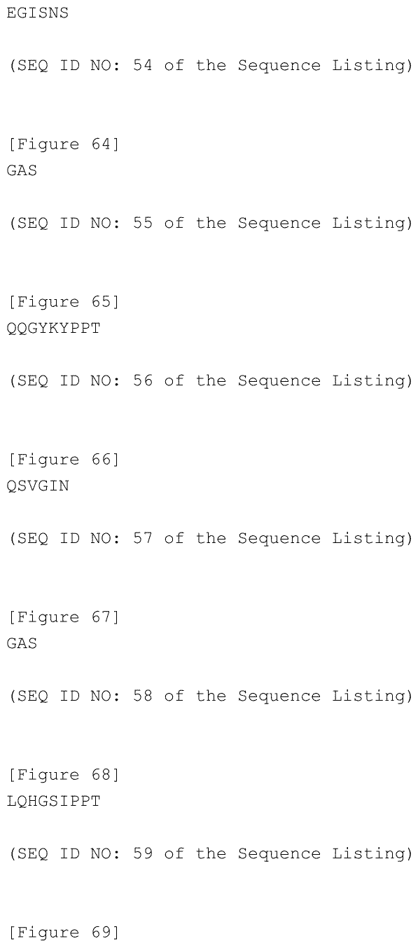

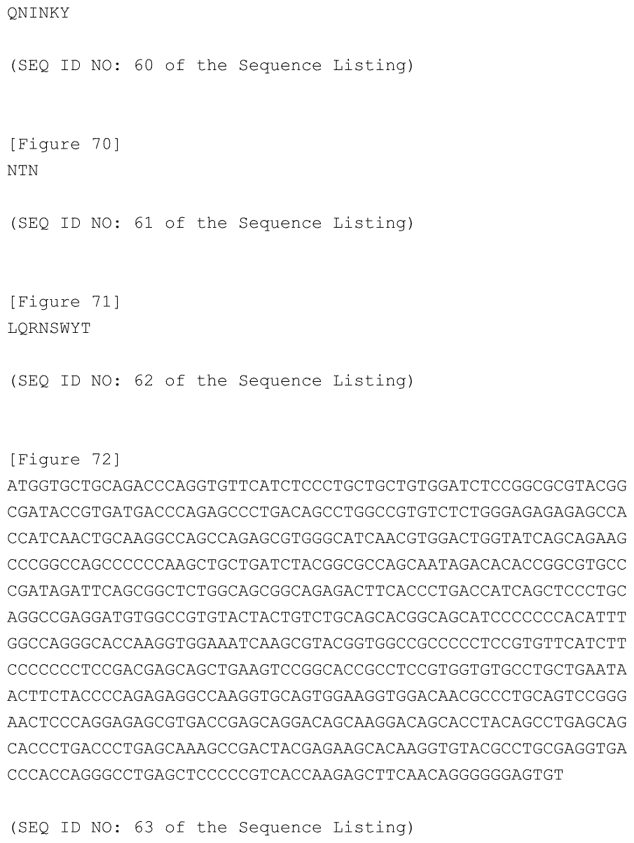

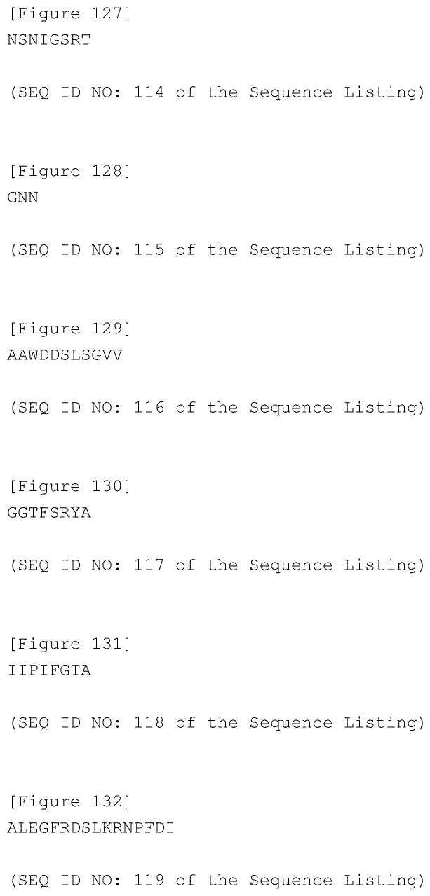

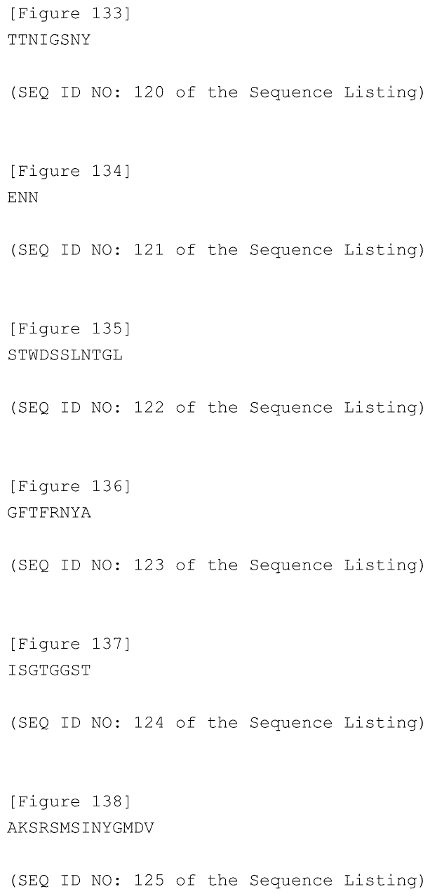

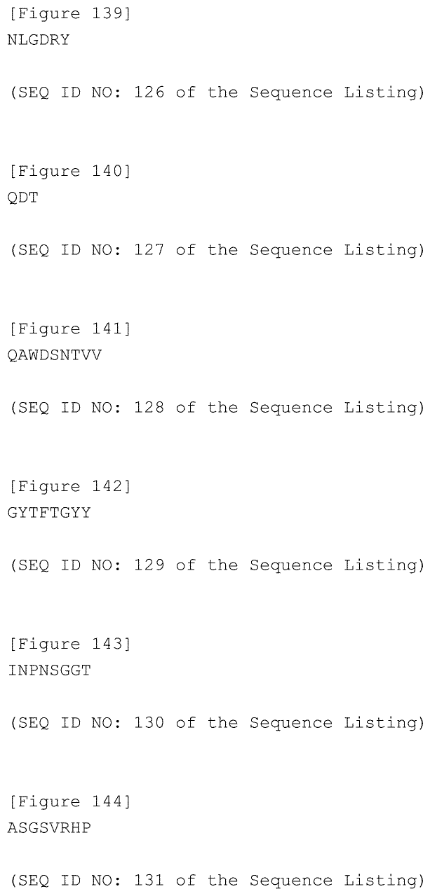

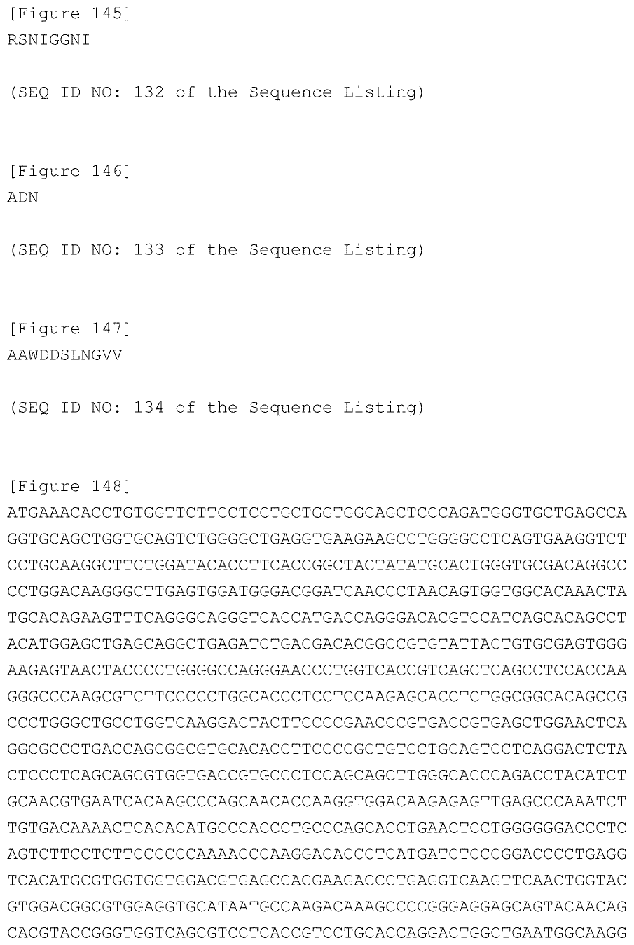

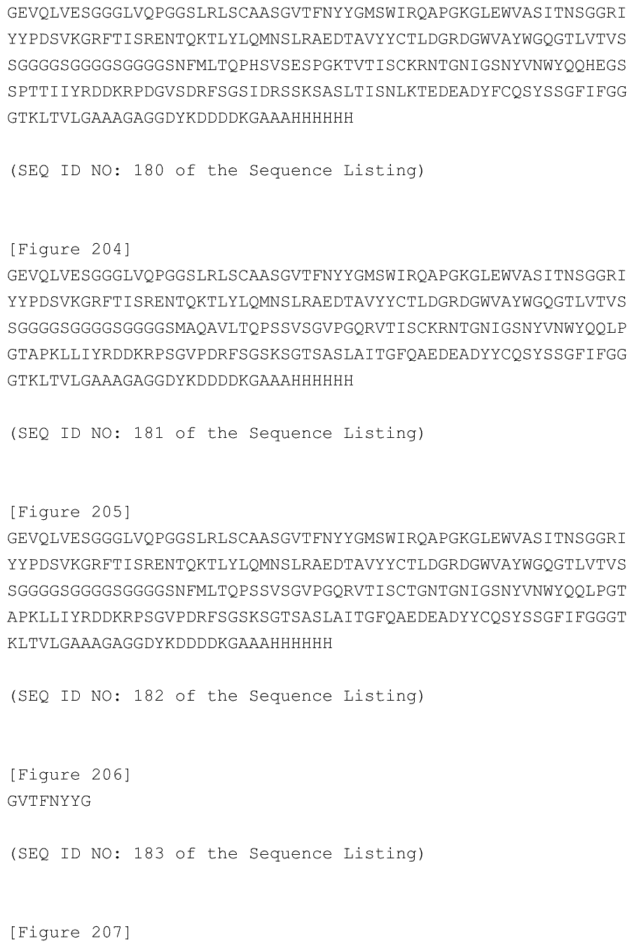

SEQ ID NO: 1: Amino-terminal sequence of human GPRC5D (FIG. 2)
SEQ ID NO: 2: Amino-terminal sequence of human GPRC5D (FIG. 3)
SEQ ID NO: 3: Primer for the PCR amplification of the variable region-encoding cDNA of the heavy chain gene of 2A4
SEQ ID NO: 4: Nucleotide sequence of a cDNA encoding the heavy chain variable region of 2A4 (FIG. 8)
SEQ ID NO: 5: Amino acid sequence of the heavy chain variable region of 2A4 (FIG. 9)
SEQ ID NO: 6: Nucleotide sequence of a cDNA encoding the heavy chain variable region of 2B1 (FIG. 10)
SEQ ID NO: 7: Amino acid sequence of the heavy chain variable region of 2B1 (FIG. 11)
SEQ ID NO: 8: Nucleotide sequence of a cDNA encoding the heavy chain variable region of 7B4 (FIG. 12)
SEQ ID NO: 9: Amino acid sequence of the heavy chain variable region of 7B4 (FIG. 13)
SEQ ID NO: 10: Primer for the PCR amplification of the variable region-encoding cDNA of the light chain gene of 2A4
SEQ ID NO: 11: Nucleotide sequence of a cDNA encoding the light chain variable region of 2A4 (FIG. 14)
SEQ ID NO: 12: Amino acid sequence of the light chain variable region of 2A4 (FIG. 15)
SEQ ID NO: 13: Nucleotide sequence of a cDNA encoding the light chain variable region of 2B1 (FIG. 16)
SEQ ID NO: 14: Amino acid sequence of the light chain variable region of 2B1 (FIG. 17)
SEQ ID NO: 15: Nucleotide sequence of a cDNA encoding the light chain variable region of 7B4 (FIG. 18)
SEQ ID NO: 16: Amino acid sequence of the light chain variable region of 7B4 (FIG. 19)
SEQ ID NO: 17: DNA fragment comprising a DNA sequence encoding the amino acids of a human κ chain secretory signal sequence and a human κ chain constant region (FIG. 20)
SEQ ID NO: 18: Primer F for a light chain expression vector (FIG. 21)
SEQ ID NO: 19: Primer R for a light chain expression vector (FIG. 22)
SEQ ID NO: 20: DNA fragment comprising a DNA sequence encoding the amino acids of a human heavy chain signal sequence and a human IgG1 constant region (FIG. 23)
SEQ ID NO: 21: Nucleotide sequence of the light chain of human chimeric 2A4 (c2A4) (FIG. 24)
SEQ ID NO: 22: Amino acid sequence of the light chain of human chimeric 2A4 (c2A4) (FIG. 25)
SEQ ID NO: 23: Primer set F for the light chain of human chimeric 2A4 (FIG. 26)
SEQ ID NO: 24: Primer set R for the light chain of human chimeric 2A4 (FIG. 27)
SEQ ID NO: 25: Nucleotide sequence of the heavy chain of human chimeric 2A4 (c2A4) (FIG. 28)
SEQ ID NO: 26: Amino acid sequence of the heavy chain of human chimeric 2A4 (c2A4) (FIG. 29)
SEQ ID NO: 27: Primer set F for the heavy chain of human chimeric 2A4 (FIG. 30)
SEQ ID NO: 28: Primer set R for the heavy chain of human chimeric 2A4 (FIG. 31)
SEQ ID NO: 29: Nucleotide sequence of the light chain of human chimeric 2B1 (c2B1) (FIG. 32)
SEQ ID NO: 30: Amino acid sequence of the light chain of human chimeric 2B1 (c2B1) (FIG. 33)
SEQ ID NO: 31: Primer set F for the light chain of human chimeric 2B1 (FIG. 34)
SEQ ID NO: 32: Primer set R for the light chain of human chimeric 2B1 (FIG. 35)
SEQ ID NO: 33: Nucleotide sequence of the heavy chain of human chimeric 2B1 (c2B1) (FIG. 36)
SEQ ID NO: 34: Amino acid sequence of the heavy chain of human chimeric 2B1 (c2B1) (FIG. 37)
SEQ ID NO: 35: Primer set F for the heavy chain of human chimeric 2B1 (FIG. 38)
SEQ ID NO: 36: Primer set R for the heavy chain of human chimeric 2B1 (FIG. 39)
SEQ ID NO: 37: Nucleotide sequence of the light chain of human chimeric 7B4 (c7B4) (FIG. 40)
SEQ ID NO: 38: Amino acid sequence of the light chain of human chimeric 7B4 (c7B4) (FIG. 41)
SEQ ID NO: 39: Primer set F for the light chain of human chimeric 7B4 (FIG. 42)
SEQ ID NO: 40: Primer set R for the light chain of human chimeric 7B4 (FIG. 43)
SEQ ID NO: 41: Nucleotide sequence of the heavy chain of human chimeric 7B4 (c7B4) (FIG. 44)
SEQ ID NO: 42: Amino acid sequence of the heavy chain of human chimeric 7B4 (c7B4) (FIG. 45)
SEQ ID NO: 43: Primer set F for the heavy chain of human chimeric 7B4 (FIG. 46)
SEQ ID NO: 44: Primer set R for the heavy chain of human chimeric 7B4 (FIG. 47)
SEQ ID NO: 45: Amino acid sequence of the heavy chain CDR1 of the rat anti-GPRC5D antibody 2A4 (FIG. 54)
SEQ ID NO: 46: Amino acid sequence of the heavy chain CDR2 of the rat anti-GPRC5D antibody 2A4 (FIG. 55)
SEQ ID NO: 47: Amino acid sequence of the heavy chain CDR3 of the rat anti-GPRC5D antibody 2A4 (FIG. 56)
SEQ ID NO: 48: Amino acid sequence of the heavy chain CDR1 of the rat anti-GPRC5D antibody 2B1 (FIG. 57)
SEQ ID NO: 49: Amino acid sequence of the heavy chain CDR2 of the rat anti-GPRC5D antibody 2B1 (FIG. 58)
SEQ ID NO: 50: Amino acid sequence of the heavy chain CDR3 of the rat anti-GPRC5D antibody 2B1 (FIG. 59)
SEQ ID NO: 51: Amino acid sequence of the heavy chain CDR1 of the rat anti-GPRC5D antibody 7B4 (FIG. 60)
SEQ ID NO: 52: Amino acid sequence of the heavy chain CDR2 of the rat anti-GPRC5D antibody 7B4 (FIG. 61)
SEQ ID NO: 53: Amino acid sequence of the heavy chain CDR3 of the rat anti-GPRC5D antibody 7B4 (FIG. 62)
SEQ ID NO: 54: Amino acid sequence of the light chain CDR1 of the rat anti-GPRC5D antibody 2A4 (FIG. 63)
SEQ ID NO: 55: Amino acid sequence of the light chain CDR2 of the rat anti-GPRC5D antibody 2A4 (FIG. 64)
SEQ ID NO: 56: Amino acid sequence of the light chain CDR3 of the rat anti-GPRC5D antibody 2A4 (FIG. 65)
SEQ ID NO: 57: Amino acid sequence of the light chain CDR1 of the rat anti-GPRC5D antibody 2B1 (FIG. 66)
SEQ ID NO: 58: Amino acid sequence of the light chain CDR2 of the rat anti-GPRC5D antibody 2B1 (FIG. 67)
SEQ ID NO: 59: Amino acid sequence of the light chain CDR3 of the rat anti-GPRC5D antibody 2B1 (FIG. 68)
SEQ ID NO: 60: Amino acid sequence of the light chain CDR1 of the rat anti-GPRC5D antibody 7B4 (FIG. 69)
SEQ ID NO: 61: Amino acid sequence of the light chain CDR2 of the rat anti-GPRC5D antibody 7B4 (FIG. 70)
SEQ ID NO: 62: Amino acid sequence of the light chain CDR3 of the rat anti-GPRC5D antibody 7B4 (FIG. 71)
SEQ ID NO: 63: Nucleotide sequence of a humanized 2B1 light chain (h2B1_L1) (FIG. 72). In this sequence, nucleotide positions 1 to 60 represent a signal sequence, which is usually not contained in the nucleotide sequences of most of mature h2B1 light chains.
SEQ ID NO: 64: Amino acid sequence of the humanized 2B1 light chain (h2B1_L1) (FIG. 73)
SEQ ID NO: 65: Nucleotide sequence of the humanized 2B1 light chain (h2B1_L2) (FIG. 74)
SEQ ID NO: 66: Amino acid sequence of the humanized 2B1 light chain (h2B1_L2) (FIG. 75)
SEQ ID NO: 67: Nucleotide sequence of the humanized 2B1 light chain (h2B1_L3) (FIG. 76)
SEQ ID NO: 68: Amino acid sequence of the humanized 2B1 light chain (h2B1_L3) (FIG. 77)
SEQ ID NO: 69: Nucleotide sequence of the humanized 2B1 light chain (h2B1_L4) (FIG. 78)
SEQ ID NO: 70: Amino acid sequence of the humanized 2B1 light chain (h2B1_L4) (FIG. 79)
SEQ ID NO: 71: Nucleotide sequence of the humanized 2B1 light chain (h2B1_L5) (FIG. 80)
SEQ ID NO: 72: Amino acid sequence of the humanized 2B1 light chain (h2B1_L5) (FIG. 81)
SEQ ID NO: 73: Nucleotide sequence of the humanized 2B1 heavy chain (h2B1_H1) (FIG. 82)
SEQ ID NO: 74: Amino acid sequence of the humanized 2B1 heavy chain (h2B1_H1) (FIG. 83)
SEQ ID NO: 75: Nucleotide sequence of the humanized 2B1 heavy chain (h2B1_H2) (FIG. 84)
SEQ ID NO: 76: Amino acid sequence of the humanized 2B1 heavy chain (h2B1_H2) (FIG. 85)
SEQ ID NO: 77: Nucleotide sequence of the humanized 2B1 heavy chain (h2B1_H3) (FIG. 86)
SEQ ID NO: 78: Amino acid sequence of the humanized 2B1 heavy chain (h2B1_H3) (FIG. 87)
SEQ ID NO: 79: Nucleotide sequence of the humanized 2B1 heavy chain (h2B1_H4) (FIG. 88)
SEQ ID NO: 80: Amino acid sequence of the humanized 2B1 heavy chain (h2B1_H4) (FIG. 89)
SEQ ID NO: 81: Nucleotide sequence of the humanized 7B4 light chain (h7B4_L1) (FIG. 90)
SEQ ID NO: 82: Amino acid sequence of the humanized 7B4 light chain (h7B4_L1) (FIG. 91)
SEQ ID NO: 83: Nucleotide sequence of the humanized 7B4 light chain (h7B4_L2) (FIG. 92)
SEQ ID NO: 84: Amino acid sequence of the humanized 7B4 light chain (h7B4_L2) (FIG. 93)
SEQ ID NO: 85: Nucleotide sequence of the humanized 7B4 heavy chain (h7B4_H1) (FIG. 94)
SEQ ID NO: 86: Amino acid sequence of the humanized 7B4 heavy chain (h7B4_H1) (FIG. 95)
SEQ ID NO: 87: Nucleotide sequence of the humanized 7B4 heavy chain (h7B4_H2) (FIG. 96)
SEQ ID NO: 88: Amino acid sequence of the humanized 7B4 heavy chain (h7B4_H2) (FIG. 97)
SEQ ID NO: 89: Nucleotide sequence of the humanized 7B4 heavy chain (h7B4_H3) (FIG. 98)
SEQ ID NO: 90: Amino acid sequence of the humanized 7B4 heavy chain (h7B4_H3) (FIG. 99)
SEQ ID NO: 91: Nucleotide sequence of the humanized 7B4 heavy chain (h7B4_H5) (FIG. 100)
SEQ ID NO: 92: Amino acid sequence of the humanized 7B4 heavy chain (h7B4_H5) (FIG. 101)
SEQ ID NO: 93: Amino acid sequence of the amino-terminal peptide of cynomolgus monkey GPRC5D (FIG. 105)
SEQ ID NO: 94: Nucleotide sequence of primer A used in the sequence analysis of scFv (FIG. 106)
SEQ ID NO: 95: Nucleotide sequence of primer B used in the sequence analysis of scFv (FIG. 107)
SEQ ID NO: 96: Nucleotide sequence of the heavy chain variable region of C2037 (FIG. 108)
SEQ ID NO: 97: Amino acid sequence of the heavy chain variable region of C2037 (FIG. 109)
SEQ ID NO: 98: Nucleotide sequence of the light chain variable region of C2037 (FIG. 110)
SEQ ID NO: 99: Amino acid sequence of the light chain variable region of C2037 (FIG. 111)
SEQ ID NO: 100: Nucleotide sequence of the heavy chain variable region of C3048 (FIG. 112)
SEQ ID NO: 101: Amino acid sequence of the heavy chain variable region of C3048 (FIG. 113)
SEQ ID NO: 102: Nucleotide sequence of the light chain variable region of C3048 (FIG. 114)
SEQ ID NO: 103: Amino acid sequence of the light chain variable region of C3048 (FIG. 115)
SEQ ID NO: 104: Nucleotide sequence of the heavy chain variable region of C3015 (FIG. 116)
SEQ ID NO: 105: Amino acid sequence of the heavy chain variable region of C3015 (FIG. 117)
SEQ ID NO: 106: Nucleotide sequence of the light chain variable region of C3015 (FIG. 118)
SEQ ID NO: 107: Amino acid sequence of the light chain variable region of C3015 (FIG. 119)
SEQ ID NO: 108: Nucleotide sequence of the heavy chain variable region of C3022 (FIG. 120)
SEQ ID NO: 109: Amino acid sequence of the heavy chain variable region of C3022 (FIG. 121)
SEQ ID NO: 110: Nucleotide sequence of the light chain variable region of C3022 (FIG. 122)
SEQ ID NO: 111: Amino acid sequence of the heavy chain CDR1 of C2037 (FIG. 124)
SEQ ID NO: 112: Amino acid sequence of the heavy chain CDR2 of C2037 (FIG. 125)
SEQ ID NO: 113: Amino acid sequence of the heavy chain CDR3 of C2037 (FIG. 126)
SEQ ID NO: 114: Amino acid sequence of the light chain CDR1 of C2037 (FIG. 127)
SEQ ID NO: 115: Amino acid sequence of the light chain CDR2 of C2037 (FIG. 128)
SEQ ID NO: 116: Amino acid sequence of the light chain CDR3 of C2037 (FIG. 129)
SEQ ID NO: 117: Amino acid sequence of the heavy chain CDR1 of C3048 (FIG. 130)
SEQ ID NO: 118: Amino acid sequence of the heavy chain CDR2 of C3048 (FIG. 131)
SEQ ID NO: 119: Amino acid sequence of the heavy chain CDR3 of C3048 (FIG. 132)
SEQ ID NO: 120: Amino acid sequence of the light chain CDR1 of C3048 (FIG. 133)
SEQ ID NO: 121: Amino acid sequence of the light chain CDR2 of C3048 (FIG. 134)
SEQ ID NO: 122: Amino acid sequence of the light chain CDR3 of C3048 (FIG. 135)
SEQ ID NO: 123: Amino acid sequence of the heavy chain CDR1 of C3015 (FIG. 136)
SEQ ID NO: 124: Amino acid sequence of the heavy chain CDR2 of C3015 (FIG. 137)
SEQ ID NO: 125: Amino acid sequence of the heavy chain CDR3 of C3015 (FIG. 138)
SEQ ID NO: 126: Amino acid sequence of the light chain CDR1 of C3015 (FIG. 139)
SEQ ID NO: 127: Amino acid sequence of the light chain CDR2 of C3015 (FIG. 140)
SEQ ID NO: 128: Amino acid sequence of the light chain CDR3 of C3015 (FIG. 141)
SEQ ID NO: 129: Amino acid sequence of the heavy chain CDR1 of C3022 (FIG. 142)
SEQ ID NO: 130: Amino acid sequence of the heavy chain CDR2 of C3022 (FIG. 143)
SEQ ID NO: 131: Amino acid sequence of the heavy chain CDR3 of C3022 (FIG. 144)
SEQ ID NO: 132: Amino acid sequence of the light chain CDR1 of C3022 (FIG. 145)
SEQ ID NO: 133: Amino acid sequence of the light chain CDR2 of C3022 (FIG. 146)
SEQ ID NO: 134: Amino acid sequence of the light chain CDR3 of C3022 (FIG. 147)
SEQ ID NO: 135: Amino acid sequence of the light chain variable region of C3022 (FIG. 123)
SEQ ID NO: 136: Nucleotide sequence of the heavy chain of an IgG form of C2037 (FIG. 148)
SEQ ID NO: 137: Nucleotide sequence of the light chain of an IgG form of C2037 (FIG. 149)
SEQ ID NO: 138: Nucleotide sequence of the heavy chain of an IgG form of C3048 (FIG. 150)
SEQ ID NO: 139: Nucleotide sequence of the light chain of an IgG form of C3048 (FIG. 151)
SEQ ID NO: 140: Nucleotide sequence of the heavy chain of an IgG form of C3015 (FIG. 152)
SEQ ID NO: 141: Nucleotide sequence of the light chain of an IgG form of C3015 (FIG. 153)
SEQ ID NO: 142: Nucleotide sequence of the heavy chain of an IgG form of C3022 (FIG. 154)
SEQ ID NO: 143: Nucleotide sequence of the light chain of an IgG form of C3022 (FIG. 155)
SEQ ID NO: 144: Amino acid sequence of the heavy chain of an IgG form of C2037 (FIG. 156)
SEQ ID NO: 145: Amino acid sequence of the light chain of an IgG form of C2037 (FIG. 157)
SEQ ID NO: 146: Amino acid sequence of the heavy chain of an IgG form of C3048 (FIG. 158)
SEQ ID NO: 147: Amino acid sequence of the light chain of an IgG form of C3048 (FIG. 159)
SEQ ID NO: 148: Amino acid sequence of the heavy chain of an IgG form of C3015 (FIG. 160)
SEQ ID NO: 149: Amino acid sequence of the light chain of an IgG form of C3015 (FIG. 161)
SEQ ID NO: 150: Amino acid sequence of the heavy chain of an IgG form of C3022 (FIG. 162)
SEQ ID NO: 151: Amino acid sequence of the light chain of the IgG form of C3022 (FIG. 163)
SEQ ID NO: 152: Nucleotide sequence of the heavy chain variable region of a rat anti-CD3 antibody (FIG. 168)
SEQ ID NO: 153: Nucleotide sequence of the light chain variable region of the rat anti-CD3 antibody (FIG. 169)
SEQ ID NO: 154: Nucleotide sequence of C3E7000 (FIG. 170)
SEQ ID NO: 155: Amino acid sequence of the heavy chain variable region of C3E7034 (FIG. 171)
SEQ ID NO: 156: Amino acid sequence of the light chain variable region of C3E7034 (FIG. 172)
SEQ ID NO: 157: Nucleotide sequence of C3E7034 (FIG. 173)
SEQ ID NO: 158: Amino acid sequence of the light chain variable region of C3E7035 (FIG. 174)
SEQ ID NO: 159: Nucleotide sequence of C3E7035 (FIG. 175)
SEQ ID NO: 160: Amino acid sequence of the light chain variable region of C3E7036 (FIG. 176)
SEQ ID NO: 161: Nucleotide sequence of C3E7036 (FIG. 177)
SEQ ID NO: 162: Nucleotide sequence of C2037-C3E7034 (FIG. 178)
SEQ ID NO: 163: Nucleotide sequence of C3048-C3E7034 (FIG. 179)
SEQ ID NO: 164: Nucleotide sequence of C3022-C3E7034 (FIG. 180)
SEQ ID NO: 165: Nucleotide sequence of C2037-C3E7035 (FIG. 181)
SEQ ID NO: 166: Nucleotide sequence of C3048-C3E7035 (FIG. 182)
SEQ ID NO: 167: Nucleotide sequence of C3022-C3E7035 (FIG. 183)
SEQ ID NO: 168: Nucleotide sequence of C2037-C3E7036 (FIG. 184)
SEQ ID NO: 169: Nucleotide sequence of C3048-C3E7036 (FIG. 185)
SEQ ID NO: 170: Nucleotide sequence of C3022-C3E7036 (FIG. 186)
SEQ ID NO: 171: Amino acid sequence of C2037-C3E7034 (FIG. 187)
SEQ ID NO: 172: Amino acid sequence of C3048-C3E7034 (FIG. 188)
SEQ ID NO: 173: Amino acid sequence of C3022-C3E7034 (FIG. 189)
SEQ ID NO: 174: Amino acid sequence of C2037-C3E7035 (FIG. 190)
SEQ ID NO: 175: Amino acid sequence of C3048-C3E7035 (FIG. 191)
SEQ ID NO: 176: Amino acid sequence of C3022-C3E7035 (FIG. 192)
SEQ ID NO: 177: Amino acid sequence of C2037-C3E7036 (FIG. 193)
SEQ ID NO: 178: Amino acid sequence of C3048-C3E7036 (FIG. 194)
SEQ ID NO: 179: Amino acid sequence of C3022-C3E7036 (FIG. 195)
SEQ ID NO: 180: Amino acid sequence of C3E7034 (FIG. 203)
SEQ ID NO: 181: Amino acid sequence of C3E7035 (FIG. 204)
SEQ ID NO: 182: Amino acid sequence of C3E7036 (FIG. 205)
SEQ ID NO: 183: Amino acid sequence of the heavy chain CDR1 of C3E7000 (FIG. 206)
SEQ ID NO: 184: Amino acid sequence of the heavy chain CDR2 of C3E7000 (FIG. 207)
SEQ ID NO: 185: Amino acid sequence of the heavy chain CDR3 of C3E7000 (FIG. 208)
SEQ ID NO: 186: Amino acid sequence of the light chain CDR1 of C3E7000 (FIG. 209)
SEQ ID NO: 187: Amino acid sequence of the light chain CDR2 of C3E7000 (FIG. 210)
SEQ ID NO: 188: Amino acid sequence of the light chain CDR3 of C3E7000 (FIG. 211)
SEQ ID NO: 189: Amino acid sequence of human CD3c (FIG. 212)
SEQ ID NO: 190: Nucleotide sequence of the heavy chain variable region of E1018 (FIG. 213)
SEQ ID NO: 191: Amino acid sequence of the heavy chain variable region of E1018 (FIG. 214)
SEQ ID NO: 192: Nucleotide sequence of the light chain variable region of E1018 (FIG. 215)
SEQ ID NO: 193: Amino acid sequence of the light chain variable region of E1018 (FIG. 216)
SEQ ID NO: 194: Nucleotide sequence of the heavy chain variable region of D1012 (FIG. 217)
SEQ ID NO: 195: Amino acid sequence of the heavy chain variable region of D1012 (FIG. 218)
SEQ ID NO: 196: Nucleotide sequence of the light chain variable region of D1012 (FIG. 219)
SEQ ID NO: 197: Amino acid sequence of the light chain variable region of D1012 (FIG. 220)
SEQ ID NO: 198: Nucleotide sequence of h2B1_Fab_HC_1 (FIG. 222)
SEQ ID NO: 199: Amino acid sequence of h2B1_Fab_HC_1 (FIG. 223)
SEQ ID NO: 200: Nucleotide sequence of h2B1_Fab_HC_2 (FIG. 224)
SEQ ID NO: 201: Amino acid sequence of h2B1_Fab_HC_2 (FIG. 225)
SEQ ID NO: 202: Nucleotide sequence of h2B1_Fab_LC_1 (FIG. 226)
SEQ ID NO: 203: Amino acid sequence of h2B1_Fab_LC_1 (FIG. 227)
SEQ ID NO: 204: Nucleotide sequence of h2B1_Fab_LC_2 (FIG. 228)
SEQ ID NO: 205: Amino acid sequence of h2B1_Fab_LC_2 (FIG. 229)
SEQ ID NO: 206: Nucleotide sequence of C3E-7034 Fab HC (FIG. 230)
SEQ ID NO: 207: Amino acid sequence of C3E-7034 Fab HC (FIG. 231)
SEQ ID NO: 208: Nucleotide sequence of C3E-7034 Fab LC (FIG. 232)
SEQ ID NO: 209: Amino acid sequence of C3E-7034 Fab LC (FIG. 233)
SEQ ID NO: 210: Nucleotide sequence of C3E-7036 Fab HC (FIG. 234)
SEQ ID NO: 211: Amino acid sequence of C3E-7036 Fab HC (FIG. 235)
SEQ ID NO: 212: Nucleotide sequence of C3E-7036 Fab LC (FIG. 236)
SEQ ID NO: 213: Amino acid sequence of C3E-7036 Fab LC (FIG. 237)
SEQ ID NO: 214: Nucleotide sequence of h2B1_Fab_HC_3 (FIG. 238)
SEQ ID NO: 215: Amino acid sequence of h2B1_Fab_HC_3 (FIG. 239)
SEQ ID NO: 216: Nucleotide sequence of h2B1_Fab_LC_3 (FIG. 240)
SEQ ID NO: 217: Amino acid sequence of h2B1_Fab_LC_3 (FIG. 241)
SEQ ID NO: 218: Nucleotide sequence of C3E-7034 scFv Fc (FIG. 242)
SEQ ID NO: 219: Amino acid sequence of C3E-7034 scFv Fc (FIG. 243)
SEQ ID NO: 220: Nucleotide sequence of C3E-7036 scFv Fc (FIG. 244)
SEQ ID NO: 221: Amino acid sequence of C3E-7036 scFv Fc (FIG. 245)
SEQ ID NO: 222: Nucleotide sequence of h2B1_scFv_Fc (FIG. 246)
SEQ ID NO: 223: Amino acid sequence of h2B1_scFv_Fc (FIG. 247)
SEQ ID NO: 224: Nucleotide sequence of C3E-8015 (FIG. 255)
SEQ ID NO: 225: Amino acid sequence of C3E-8015 (FIG. 256)
SEQ ID NO: 226: Nucleotide sequence of C3E-8017 (FIG. 257)
SEQ ID NO: 227: Amino acid sequence of C3E-8017 (FIG. 258)
SEQ ID NO: 228: Nucleotide sequence of C3E-8018 (FIG. 259)
SEQ ID NO: 229: Amino acid sequence of C3E-8018 (FIG. 260)
SEQ ID NO: 230: Nucleotide sequence of C3E-8025 (FIG. 261)
SEQ ID NO: 231: Amino acid sequence of C3E-8025 (FIG. 262)
SEQ ID NO: 232: Nucleotide sequence of C3E-8027 (FIG. 263)
SEQ ID NO: 233: Amino acid sequence of C3E-8027 (FIG. 264)
SEQ ID NO: 234: Nucleotide sequence of C3E-8028 (FIG. 265)
SEQ ID NO: 235: Amino acid sequence of C3E-8028 (FIG. 266)
SEQ ID NO: 236: Nucleotide sequence of h2B1_Fab_HC_4 (FIG. 267)
SEQ ID NO: 237: Amino acid sequence of h2B1_Fab_HC_4 (FIG. 268)
SEQ ID NO: 238: Amino acid sequence of the heavy chain CDR2 of a CDR-modified form (FIG. 276) wherein X is an arbitrary natural amino acid.
SEQ ID NO: 239: Amino acid sequence of the light chain CDR2 of the CDR-modified form (FIG. 277) wherein X is an arbitrary natural amino acid.
SEQ ID NO: 240: Amino acid sequence of the heavy chain variable region of a CDR-modified form of C3E-7034 (FIG. 278) wherein X is an arbitrary natural amino acid.
SEQ ID NO: 241: Amino acid sequence of the light chain variable region of the CDR-modified form of C3E-7034 (FIG. 279) wherein X is an arbitrary natural amino acid.
SEQ ID NO: 242: Amino acid sequence of the light chain variable region of a CDR-modified form of C3E-7035 (FIG. 280) wherein X is an arbitrary natural amino acid.
SEQ ID NO: 243: Amino acid sequence of the light chain variable region of a CDR-modified form of C3E-7036 (FIG. 281) wherein X is an arbitrary natural amino acid.
SEQ ID NO: 244: Amino acid sequence of C3E-7078 (FIG. 282)
SEQ ID NO: 245: Amino acid sequence of C3E-7085 (FIG. 283)
SEQ ID NO: 246: Amino acid sequence of C3E-7086 (FIG. 284)
SEQ ID NO: 247: Amino acid sequence of C3E-7087 (FIG. 285)
SEQ ID NO: 248: Amino acid sequence of C3E-7088 (FIG. 286)
SEQ ID NO: 249: Amino acid sequence of C3E-7089 (FIG. 287)
SEQ ID NO: 250: Amino acid sequence of C3E-7090 (FIG. 288)
SEQ ID NO: 251: Amino acid sequence of C3E-7091 (FIG. 289)
SEQ ID NO: 252: Amino acid sequence of C3E-7092 (FIG. 290)
SEQ ID NO: 253: Amino acid sequence of C3E-7093 (FIG. 291)
SEQ ID NO: 254: Amino acid sequence of C3E-7094 (FIG. 292)
SEQ ID NO: 255: Amino acid sequence of C3E-7095 (FIG. 293)

1. An antibody or an antigen-binding fragment of the antibody, wherein the antibody comprises a heavy chain variable region and a light chain variable region described in any one of the following (I), (II), or (III):
(II)
a heavy chain variable region comprising
heavy chain CDR1 consisting of the amino acid sequence represented by SEQ ID NO: 48,
heavy chain CDR2 consisting of the amino acid sequence represented by SEQ ID NO: 49, and
heavy chain CDR3 consisting of the amino acid sequence represented by SEQ ID NO: 50, and
a light chain variable region comprising
light chain CDR1 consisting of the amino acid sequence represented by SEQ ID NO: 57,
light chain CDR2 consisting of the amino acid sequence represented by SEQ ID NO: 58, and
light chain CDR3 consisting of the amino acid sequence represented by SEQ ID NO: 59,
(I)
a heavy chain variable region comprising
heavy chain CDR1 consisting of the amino acid sequence represented by SEQ ID NO: 45,
heavy chain CDR2 consisting of the amino acid sequence represented by SEQ ID NO: 46, and
heavy chain CDR3 consisting of the amino acid sequence represented by SEQ ID NO: 47, and
a light chain variable region comprising
light chain CDR1 consisting of the amino acid sequence represented by SEQ ID NO: 54,
light chain CDR2 consisting of the amino acid sequence represented by SEQ ID NO: 55, and
light chain CDR3 consisting of the amino acid sequence represented by SEQ ID NO: 56,and
(III)
a heavy chain variable region comprising
heavy chain CDR1 consisting of the amino acid sequence represented by SEQ ID NO: 51,
heavy chain CDR2 consisting of the amino acid sequence represented by SEQ ID NO: 52, and
heavy chain CDR3 consisting of the amino acid sequence represented by SEQ ID NO: 53, and
a light chain variable region comprising
light chain CDR1 consisting of the amino acid sequence represented by SEQ ID NO: 60,
light chain CDR2 consisting of the amino acid sequence represented by SEQ ID NO: 61, and
light chain CDR3 consisting of the amino acid sequence represented by SEQ ID NO: 62
and binds to human GPRC5D.

2. The antibody or antigen-binding fragment of the antibody according to claim 1, wherein the heavy chain variable region and the light chain variable region are the heavy chain variable region and the light chain variable region described in (II).

3. The antibody or antigen-binding fragment of the antibody according to claim 1, wherein the heavy chain variable region and the light chain variable region are the heavy chain variable region and the light chain variable region described in (I).

4. The antibody or antigen-binding fragment of the antibody according to claim 1, wherein the heavy chain variable region and the light chain variable region are the heavy chain variable region and the light chain variable region described in (III).

5. The antibody or antigen-binding fragment of the antibody according to claim 1, wherein the antibody or antigen-binding fragment is a chimeric antibody or an antigen-binding fragment of the antibody.

6. The antibody or antigen-binding fragment of the antibody according to claim 1, wherein the antibody or antigen-binding fragment is a humanized antibody or an antigen-binding fragment of the antibody.

7. The antibody or antigen-binding fragment of the antibody according to claim 1, wherein the antibody or antigen-binding fragment is a human antibody or an antigen-binding fragment of the antibody.

8. The antibody or antigen-binding fragment of the antibody according to claim 1, wherein
the light chain variable region comprising any one amino acid sequence of
amino acid residues 21 to 127 of SEQ ID NO: 64,
amino acid residues 21 to 127 of SEQ ID NO: 66,
amino acid residues 21 to 127 of SEQ ID NO: 68,
amino acid residues 21 to 127 of SEQ ID NO: 70, and
amino acid residues 21 to 127 of SEQ ID NO: 72, and
the heavy chain variable region comprising any one amino acid sequence of
amino acid residues 20 to 142 of SEQ ID NO: 74,
amino acid residues 20 to 142 of SEQ ID NO: 76,
amino acid residues 20 to 142 of SEQ ID NO: 78, and
amino acid residues 20 to 142 of SEQ ID NO: 80.

9. The antibody or antigen-binding fragment of the antibody according to claim 1, wherein the antibody comprises any one combination of:
the heavy chain variable region comprises amino acid residues 20 to 142 of SEQ ID NO: 74, and
the light chain variable region comprises amino acid residues 21 to 127 of SEQ ID NO: 64,
the heavy chain variable region comprises amino acid residues 20 to 142 of SEQ ID NO: 74, and
the light chain variable region comprises amino acid residues 21 to 127 of SEQ ID NO: 66,
the heavy chain variable region comprises amino acid residues 20 to 142 of SEQ ID NO: 76, and
the light chain variable region comprises amino acid residues 21 to 127 of SEQ ID NO: 66,
the heavy chain variable region comprises amino acid residues 20 to 142 of SEQ ID NO: 76, and
the light chain variable region comprises amino acid residues 21 to 127 of SEQ ID NO: 68,
the heavy chain variable region comprises amino acid residues 20 to 142 of SEQ ID NO: 76, and
the light chain variable region amino acid residues 21 to 127 of SEQ ID NO: 70,
the heavy chain variable region comprises amino acid residues 20 to 142 of SEQ ID NO: 76, and
the light chain variable region comprises amino acid residues 21 to 127 of SEQ ID NO: 72,
the heavy chain variable region comprises amino acid residues 20 to 142 of SEQ ID NO: 78, and
the light chain variable region comprises amino acid residues 21 to 127 of SEQ ID NO: 68,
the heavy chain variable region comprises amino acid residues 20 to 142 of SEQ ID NO: 78, and
the light chain variable region amino acid residues 21 to 127 of SEQ ID NO: 70,
the heavy chain variable region comprises amino acid residues 20 to 142 of SEQ ID NO: 78, and
the light chain variable region comprises amino acid residues 21 to 127 of SEQ ID NO: 72,
the heavy chain variable region comprises amino acid residues 20 to 142 of SEQ ID NO: 80, and
the light chain variable region comprises amino acid residues 21 to 127 of SEQ ID NO: 64,
the heavy chain variable region comprises amino acid residues 20 to 142 of SEQ ID NO: 80, and
the light chain variable region comprises amino acid residues 21 to 127 of SEQ ID NO: 68,
the heavy chain variable region comprises amino acid residues 20 to 142 of SEQ ID NO: 80, and
the light chain variable region comprises amino acid residues 21 to 127 of SEQ ID NO: 70, and
the heavy chain variable region comprises amino acid residues 20 to 142 of SEQ ID NO: 80, and
the light chain variable region comprises amino acid residues 21 to 127 of SEQ ID NO: 72.

10. The antibody or antigen-binding fragment of the antibody according to claim 1, wherein the light chain variable region comprises
amino acid residues 21 to 126 of SEQ ID NO: 82, or
amino acid residues 21 to 126 of SEQ ID NO: 84, and
the heavy chain variable region comprises
amino acid residues 20 to 142 of SEQ ID NO: 86,
amino acid residues 20 to 142 of SEQ ID NO: 88,
amino acid residues 20 to 142 of SEQ ID NO: 90, or
amino acid residues 20 to 142 of SEQ ID NO: 92.

11. The antibody or antigen-binding fragment of the antibody according to claim 1, wherein the antibody comprises any one combination of:
the heavy chain variable region comprises amino acid residues 20 to 142 of SEQ ID NO: 86, and
the light chain variable region comprises amino acid residues 21 to 126 of SEQ ID NO: 84,
the heavy chain variable region comprises amino acid residues 20 to 142 of SEQ ID NO: 88, and
the light chain variable region comprises amino acid residues 21 to 126 of SEQ ID NO: 84,
the heavy chain variable region comprises amino acid residues 20 to 142 of SEQ ID NO: 90, and
the light chain variable region comprises amino acid residues 21 to 126 of SEQ ID NO: 82,
the heavy chain variable region comprises amino acid residues 20 to 142 of SEQ ID NO: 90, and a
light chain variable region comprises amino acid residues 21 to 126 of SEQ ID NO: 84, and
the heavy chain variable region comprises amino acid residues 20 to 142 of SEQ ID NO: 92, and
the light chain variable region comprises amino acid residues 21 to 126 of SEQ ID NO: 82,

12. The antibody according to claim 1, wherein the antibody comprises Fc.

13. An antibody or an antigen-binding fragment of the antibody, wherein the antibody binds to human GPRC5D and comprises a heavy chain variable region and a light chain variable region described in any one of the following (A) to (D):
(A)
a heavy chain variable region comprising
heavy chain CDR1 consisting of SEQ ID NO: 111,
heavy chain CDR2 consisting of SEQ ID NO: 112, and
heavy chain CDR3 consisting of SEQ ID NO: 113, and
a light chain variable region comprising
light chain CDR1 consisting of SEQ ID NO: 114,
light chain CDR2 consisting of SEQ ID NO: 115, and
light chain CDR3 consisting of SEQ ID NO: 116,
(B)
a heavy chain variable region comprising
heavy chain CDR1 consisting of SEQ ID NO: 117,
heavy chain CDR2 consisting of SEQ ID NO: 118, and
heavy chain CDR3 consisting of the amino acid sequence r-presented by SEQ ID NO: 119, and
a light chain variable region comprising
light chain CDR1 consisting of SEQ ID NO: 120,
light chain CDR2 consisting of SEQ ID NO: 121, and
light chain CDR3 consisting of the amino acid sequence represented b) SEQ ID NO: 122,
(C)
a heavy chain variable region comprising
heavy chain CDR1 consisting of SEQ ID NO: 123,
heavy chain CDR2 consisting of SEQ ID NO: 124, and
heavy chain CDR3 consisting of SEQ ID NO: 125, and
a light chain variable region comprising
light chain CDR1 consisting of SEQ ID NO: 126,
light chain CDR2 consisting of SEQ ID NO: 127, and
light chain CDR3 consisting of SEQ ID NO: 128, and
(D)
a heavy chain variable region comprising
heavy chain CDR1 consisting of SEQ ID NO: 129,
heavy chain CDR2 consisting of the amino acid-sequence represented by SEQ ID NO: 130, and
heavy chain CDR3 consisting of SEQ ID NO: 131, and
a light chain variable region comprising
light chain CDR1 consisting of SEQ ID NO: 132,
light chain CDR2 consisting of SEQ ID NO: 133, and
light chain CDR3 consisting of SEQ ID NO: 134.

14. The antibody or antigen-binding fragment of the antibody according to claim 13, wherein the heavy chain variable region and the light chain variable region are the heavy chain variable region and the light chain variable region described in (A).

15. The antibody or antigen-binding fragment of the antibody according to claim 13, wherein the heavy chain variable region and the light chain variable region are the heavy chain variable region and the light chain variable region described in (B).

16. The antibody or antigen-binding fragment of the antibody according to claim 13, wherein the heavy chain variable region and the light chain variable region are the heavy chain variable region and the light chain variable region described in (C).

17. The antibody or antigen-binding fragment of the antibody according to claim 13, wherein the heavy chain variable region and the light chain variable region are the heavy chain variable region and the light chain variable region described in (D).

18. The antibody or antigen-binding fragment of the antibody according to claim 13, wherein the antibody comprises
the heavy chain variable region comprises any one of
SEQ ID NO: 97,
SEQ ID NO: 101,
SEQ ID NO: 105, or
SEQ ID NO: 109, and
the light chain variable region comprises any one of
SEQ ID NO: 99,
SEQ ID NO: 103,
SEQ ID NO: 107, or
SEQ ID NO: 135.

19. The antibody or antigen-binding fragment of the antibody according to claim 13, wherein the antibody comprises any one combination of:
the heavy chain variable region comprises SEQ ID NO: 97, and the light chain variable region comprises SEQ ID NO: 99,
the heavy chain variable region comprises SEQ ID NO: 101, and the light chain variable region comprises SEQ ID NO: 103,
the heavy chain variable region comprises SEQ ID NO: 105, and the light chain variable region comprises SEQ ID NO: 107, and
the heavy chain variable region comprises SEQ ID NO: 109, and the light chain variable region comprises SEQ ID NO: 135.

20. An antibody or an antigen-binding fragment of the antibody, wherein the antibody comprises any one combination of a heavy chain and a light chain of
a heavy chain comprising SEQ ID NO: 144, and a light chain comprising SEQ ID NO: 145,
a heavy chain comprising SEQ ID NO: 146, and a light chain comprising SEQ ID NO: 147,
a heavy chain comprising SEQ ID NO: 148, and a light chain comprising SEQ ID NO: 149, and
a heavy chain comprising SEQ ID NO: 150, and a light chain comprising SEQ ID NO: 151.

21. An antibody or an antigen-binding fragment of the antibody, wherein the antibody or antigen-binding fragment comprises an amino acid sequence encoded by a nucleotide sequence contained in a polynucleotide hybridizing under stringent conditions to a complementary strand of a polynucleotide comprising a nucleotide sequence encoding an amino acid sequence contained in an antibody or an antigen-binding fragment of the antibody according to claim 18.

22. An antibody or an antigen-binding fragment of the antibody, wherein the antibody or antigen-binding fragment comprises an amino acid sequence 90% or more identical to an amino acid sequence contained in an antibody or an antigen-binding fragment of the antibody according to claim 18.

23. An antibody or an antigen-binding fragment of the antibody, wherein the antibody or antigen-binding fragment comprises an amino acid sequence derived by the substitution, deletion, or addition of 1 to several amino acid(s) from an amino acid sequence contained in an antibody or an antigen-binding fragment of the antibody according to claim 18.

24. An antibody or an antigen-binding fragment of the antibody, wherein the antibody or antigen-binding fragment binds to a site on human GPRC5D bound by an antibody or an antigen-binding fragment of the antibody according to claim 1.

25. An antibody or an antigen-binding fragment of the antibody, wherein the antibody or antigen-binding fragment competes with an antibody or an antigen-binding fragment of the antibody according to claim 1.

26. The antibody or antigen-binding fragment according to claim 1, wherein the antibody or antigen-binding fragment binds to cynomolgus monkey GPRC5D.

27. The antibody or antigen-binding fragment of the antibody according to claim 1, wherein the antigen-binding fragment is Fab, F(ab)′, Fv, scFv, or sdAb.

28. An antibody or an antigen-binding fragment of the antibody, wherein the antibody comprises a heavy chain variable region and a light chain variable region described according to claim 2, and comprises
i) a heavy chain constant region comprising amino acid residues 147 to 475 of SEQ ID NO: 199, and a light chain constant region comprising amino acid residues 131 to 237 of SEQ ID NO: 203,
ii) a heavy chain constant region comprising amino acid residues 147 to 475 of SEQ ID NO: 201, and a light chain constant region comprising amino acid residues 131 to 237 of SEQ ID NO: 205,
iii) a heavy chain constant region comprising amino acid residues 147 to 475 of SEQ ID NO: 215, and a light chain constant region comprising amino acid residues 131 to 237 of SEQ ID NO: 217, or
iv) a heavy chain constant region comprising amino acid residues 147 to 475 of SEQ ID NO: 237, and a light chain constant region comprising amino acid residues 131 to 237 of SEQ ID NO: 217.

29. An antibody or an antigen-binding fragment of the antibody, wherein the antibody comprises a heavy chain variable region comprising amino acid residues 20 to 142 of SEQ ID NO: 76 and a light chain variable region comprising amino acid residues 21 to 127 of SEQ ID NO: 72, and comprising
i) a heavy chain constant region comprising amino acid residues 147 to 475 of SEQ ID NO: 199, and a light chain constant region comprising amino acid residues 131 to 237 of SEQ ID NO: 203,
ii) a heavy chain constant region comprising amino acid residues 147 to 475 of SEQ ID NO: 201, and a light chain constant region comprising amino acid residues 131 to 237 of SEQ ID NO: 205,
iii) a heavy chain constant region comprising amino acid residues 147 to 475 of SEQ ID NO: 215, and a light chain constant region comprising amino acid residues 131 to 237 of SEQ ID NO: 217, or
iv) a heavy chain constant region comprising amino acid residues 147 to 475 of SEQ ID NO: 237, and a light chain constant region comprising amino acid residues 131 to 237 of SEQ ID NO: 217.

30. An antibody or an antigen-binding fragment of the antibody, wherein the antibody comprises a heavy chain variable region and a light chain variable region described according to claim 2, and comprising mutated Fc.

31. An antibody or an antigen-binding fragment of the antibody, wherein the antibody comprises a heavy chain variable region comprising amino acid residues 20 to 142 of SEQ ID NO: 76 and a light chain variable region comprising amino acid residues 21 to 127 of SEQ ID NO: 72, and comprising mutated Fc.

32. A polynucleotide encoding an antibody or an antigen-binding fragment of the antibody according to claim 1.

33. A vector comprising the polynucleotide according to claim 32.

34. A cell comprising the polynucleotide according to claim 32.

35. An artificial immunocyte comprising expressing an antibody or an antigen-binding fragment of the antibody according to claim 1 on the cell surface.

36. A method for producing an antibody or an antigen-binding fragment of the antibody which binds to human GPRC5D, comprising the steps of: culturing a cell according to claim 34; and recovering an antibody or an antigen-binding fragment of the antibody which binds to human GPRC5D from the cultures.

37. An antibody or an antigen-binding fragment of the antibody which binds to human GPRC5D, the antibody or antigen-binding fragment being obtained by a method according to claim 36.

38. A pharmaceutical composition comprising an antibody or an antigen-binding fragment of the antibody according to claim 1 as an active ingredient, and a pharmaceutically acceptable carrier.

39. A method of treating a cancer comprising administering to a subject in need thereof the pharmaceutical composition of claim 38.

40. The method according to claim 39, wherein the cancer is breast cancer, endometrial cancer, ovary cancer, lung cancer, stomach cancer, prostate cancer, kidney cancer, liver cancer, pancreatic cancer, colorectal cancer, esophageal cancer, urinary bladder cancer, uterine cervix cancer, blood cancer, lymphoma, or malignant melanoma expressing a GPRC5D protein.

41. The method according to claim 40, wherein the cancer is multiple myeloma expressing a GPRC5D protein.

42. A molecule having antigen binding activity, comprising an antibody or an antigen-binding fragment of the antibody according to claim 1.

43. The molecule according to claim 42, wherein the molecule is multispecific.

44. The molecule according to claim 42, wherein the molecule further comprises
an antibody or an antigen-binding fragment of the antibody that comprises
a heavy chain variable region comprising
a heavy chain CDR1 comprising SEQ ID NO: 183,
a heavy chain CDR2 comprising SEQ ID NO: 238, and
a heavy chain CDR3 comprising SEQ ID NO: 185, and
a light chain variable region comprising
a light chain CDR1 comprising SEQ ID NO: 186,
a light chain CDR2 comprising SEQ ID NO: 239, and
a light chain CDR3 comprising SEQ ID NO: 188,
and binds to human CD3 and cynomolgus monkey CD3.

45. The molecule according to claim 44, wherein, in the heavy chain CDR2, the first Xaa is selected from the group consisting of A, E, G, H, I, L, T, V, R, and S, and the second Xaa is S; or the first Xaa is N, and the second Xaa is selected from the group consisting of E, R, F, Y, L, V, I, K, and T, and in the light chain CDR2, Xaa is selected from the group consisting of Q, M, A, G, S, N, and D.

46. The molecule according to claim 44, wherein, in the heavy chain CDR2, the first Xaa is selected from the group consisting of R and S, and the second Xaa is S, and in the light chain CDR2, Xaa is selected from the group consisting of Q, A, G, S, N, and D.

47. The molecule according to claim 42, wherein the antibody or antigen-binding fragment of the antibody comprises a heavy chain variable region comprising SEQ ID NO: 240 and a light chain variable region comprising any one of SEQ ID NOs: 241, 242, or 243;
wherein in SEQ ID NO: 240, the first Xaa is selected from the group consisting of A, E, G, H, I, L, T, V, R, and S, and the second Xaa is S; or the first Xaa is N, and the second Xaa is selected from the group consisting of E, R, F, Y, L, V, I, K, and T,
and in any one of SEQ ID NOs: 241, 242, and 243, Xaa is selected from the group consisting of Q, A, G, S, N, and D.

48. The molecule according to claim 47, wherein the first Xaa is selected from the group consisting of R and S, and the second Xaa is S in SEQ ID NO: 240, and Xaa is selected from the group consisting of Q, A, G, S, N, and D in any one of SEQ ID NOs: 241, 242, and 243.

49. The molecule according to claim 42, wherein the molecule comprises an antibody or an antigen-binding fragment of the antibody according to claim 1, and further comprises
an antibody or an antigen-binding fragment of the antibody that comprises
a heavy chain variable region comprising
a heavy chain CDR1 comprising SEQ ID NO: 183,
a heavy chain CDR2 comprising SEQ ID NO: 184, and
a heavy chain CDR3 comprising SEQ ID NO: 185, and
a light chain variable region comprising
a light chain CDR1 comprising SEQ ID NO: 186,
a light chain CDR2 comprising SEQ ID NO: 187, and
a light chain CDR3 comprising SEQ ID NO: 188,
and binds to human CD3 and cynomolgus monkey CD3.

50. The molecule according to claim 49, wherein the antibody that binds to human CD3 and cynomolgus monkey CD3 or antigen-binding fragment of the antibody comprises a heavy chain variable region comprising SEQ ID NO: 155 and a light chain variable region comprising any one of SEQ ID NOs: 156, 158, or 160.

51. The molecule according to claim 44, wherein the antigen-binding fragment of the antibody that binds to human CD3 and cynomolgus monkey CD3 is Fab, F(ab)′, Fv, scFv, or sdAb.

52. The molecule according to claim 44, wherein the antibody that binds to human CD3 and cynomolgus monkey CD3 is a humanized antibody or a human antibody comprising a human immunoglobulin constant region or Fc or mutated Fc.

53. The molecule according to claim 44, wherein the antibody that binds to human CD3 and cynomolgus monkey CD3 or antigen-binding fragment of the antibody comprises any one of SEQ ID NOs: 180, 181, or 182.

54. The molecule according to claim 49, wherein the antibody that binds to human CD3 and cynomolgus monkey CD3 or antigen-binding fragment of the antibody and the antibody or antigen-binding fragment of the antibody that binding to human GPRC5D are bound together via a linker or without a linker.

55. A molecule according to claim 42, wherein the molecule comprises an antibody that binds to human GPRC5D or an antigen-binding fragment of the antibody comprises a heavy chain variable region comprising
heavy chain CDR1 consisting of SEQ ID NO: 48,
heavy chain CDR2 consisting of SEQ ID NO: 49, and
heavy chain CDR3 consisting of SEQ ID NO: 50, and
a light chain variable region comprising
light chain CDR1 consisting of SEQ ID NO: 57,
light chain CDR2 consisting of SEQ ID NO: 58, and
light chain CDR3 consisting of SEQ ID NO: 59, and the molecule further comprises
an antibody that binds to human CD3 and cynomolgus monkey CD3 or an antigen-binding fragment of the antibody comprising
a heavy chain variable region comprising amino acid residues 25 to 142 of SEQ ID NO: 207 and a light chain variable region comprising amino acid residues 24 to 132 of SEQ ID NO: 209,
a heavy chain variable region comprising amino acid residues 25 to 142 of SEQ ID NO: 211 and a light chain variable region comprising amino acid residues 24 to 130 of SEQ ID NO: 213,
a heavy chain variable region comprising amino acid residues 2 to 119 of SEQ ID NO: 244 and a light chain variable region comprising amino acid residues 135 to 243 of SEQ ID NO: 244,
a heavy chain variable region comprising amino acid residues 2 to 119 of SEQ ID NO: 245 and a light chain variable region comprising amino acid residues 135 to 241 of SEQ ID NO: 245,
a heavy chain variable region comprising amino acid residues 2 to 119 of SEQ ID NO: 246 and a light chain variable region comprising amino acid residues 135 to 243 of SEQ ID NO: 246,
a heavy chain variable region comprising amino acid residues 2 to 119 of SEQ ID NO: 247 and a light chain variable region comprising amino acid residues 135 to 243 of SEQ ID NO: 247,
a heavy chain variable region comprising amino acid residues 2 to 119 of SEQ ID NO: 248 and a light chain variable region comprising amino acid residues 135 to 243 of SEQ ID NO: 248,
a heavy chain variable region comprising amino acid residues 2 to 119 of SEQ ID NO: 249 and a light chain variable region comprising amino acid residues 135 to 243 of SEQ ID NO: 249,
a heavy chain variable region comprising amino acid residues 2 to 119 of SEQ ID NO: 250 and a light chain variable region comprising amino acid residues 135 to 243 of SEQ ID NO: 250,
a heavy chain variable region comprising amino acid residues 2 to 119 of SEQ ID NO: 251 and a light chain variable region comprising amino acid residues 135 to 243 of SEQ ID NO: 251,
a heavy chain variable region comprising amino acid residues 2 to 119 of SEQ ID NO: 252 and a light chain variable region comprising amino acid residues 135 to 243 of SEQ ID NO: 252,
a heavy chain variable region comprising amino acid residues 2 to 119 of SEQ ID NO: 253 and a light chain variable region comprising amino acid residues 135 to 243 of SEQ ID NO: 253,
a heavy chain variable region comprising amino acid residues 2 to 119 of SEQ ID NO: 254 and a light chain variable region comprising amino acid residues 135 to 243 of SEQ ID NO: 254, or
a heavy chain variable region comprising amino acid residues 2 to 119 of SEQ ID NO: 255 and a light chain variable region comprising amino acid residues 135 to 243 of SEQ ID NO: 255.

56. The molecule according to claim 55 wherein the antibody that binds to human GPRC5D or an antigen-binding fragment of the antibody comprises a heavy chain variable region comprising amino acid residues 20 to 142 of SEQ ID NO: 76 and a light chain variable region comprising amino acid residues 21 to 127 of SEQ ID NO: 72, and comprises
i) a heavy chain constant region comprising amino acid residues 147 to 475 of SEQ ID NO: 199, and a light chain constant region comprising amino acid residues 131 to 237 of SEQ ID NO: 203,
ii) a heavy chain constant region comprising amino acid residues 147 to 475 of SEQ ID NO: 201, and a light chain constant region comprising amino acid residues 131 to 237 of SEQ ID NO: 205,
iii) a heavy chain constant region comprising amino acid residues 147 to 475 of SEQ ID NO: 215, and a light chain constant region comprising amino acid residues 131 to 237 of SEQ ID NO: 217, or
iv) a heavy chain constant region comprising amino acid residues 147 to 475 of SEQ ID NO: 237, and a light chain constant region comprising amino acid residues 131 to 237 of SEQ ID NO: 217, and
the antibody that binds to human CD3 and cynomolgus monkey CD3 or antigen-binding fragment of the antibody comprises mutated Fc.

57. The molecule according to claim 55 wherein the antibody that binds to human GPRC5D or an antigen-binding fragment of the antibody comprises a heavy chain variable region comprising amino acid residues 20 to 142 of SEQ ID NO: 76 and a light chain variable region comprising amino acid residues 21 to 127 of SEQ ID NO: 72, and comprises
i) a heavy chain constant region comprising amino acid residues 147 to 475 of SEQ ID NO: 215, and a light chain constant region comprising amino acid residues 131 to 237 of SEQ ID NO: 217, or
ii) a heavy chain constant region comprising amino acid residues 147 to 475 of SEQ ID NO: 237, and a light chain constant region comprising amino acid residues 131 to 237 of SEQ ID NO: 217, and
the antibody that binds to human CD3 and cynomolgus monkey CD3 or antigen-binding fragment of the antibody comprises mutated Fc.

58. The molecule according to claim 55, wherein
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 266 of SEQ ID NO: 219 and mutated Fc;
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 264 of SEQ ID NO: 221 and mutated Fc;
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 264 of SEQ ID NO: 225 and mutated Fc;
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 266 of SEQ ID NO: 227 and mutated Fc;
the antibody that binds to human GPRC5D or an antigen-binding fragment of the antibody comprises a heavy chain comprising 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 266 of SEQ ID NO: 229 and mutated Fc;
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 264 of SEQ ID NO: 231 and mutated Fc;
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 266 of SEQ ID NO: 233 and mutated Fc; or
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 266 of SEQ ID NO: 235 and mutated Fc.

59. The molecule according to claim 55, wherein
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 264 of SEQ ID NO: 225 and mutated Fc;
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 266 of SEQ ID NO: 227 and mutated Fc;
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 266 of SEQ ID NO: 229 and mutated Fc;
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 264 of SEQ ID NO: 231 and mutated Fc;
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 266 of SEQ ID NO: 233 and mutated Fc; or
the antibody that binds to human GPRC5D or an antigen-binding fragment of the antibody comprises a heavy chain comprising amino acid residues 24 to 475 of SEQ ID NO: 215 and a light chain comprising amino acid residues 24 to 237 of SEQ ID NO: 217, and
the antibody that binds to human CD3 and cynomolgus monkey CD3 or antigen-binding fragment of the antibody comprises amino acid residues 24 to 266 of SEQ ID NO: 235 and mutated Fc.

60. The molecule according to claim 55, wherein the antibody that binds to human GPRC5D or an antigen-binding fragment of the antibody comprises a heavy chain variable region comprising amino acid residues 20 to 142 of SEQ ID NO: 76 and a light chain variable region comprising amino acid residues 21 to 127 of SEQ ID NO: 72, and comprises
i) a heavy chain constant region comprising amino acid residues 147 to 475 of SEQ ID NO: 199, and a light chain constant region comprising amino acid residues 131 to 237 of SEQ ID NO: 203,
ii) a heavy chain constant region comprising amino acid residues 147 to 475 of SEQ ID NO: 201, and a light chain constant region comprising amino acid residues 131 to 237 of SEQ ID NO: 205,
iii) a heavy chain constant region comprising amino acid residues 147 to 475 of SEQ ID NO: 215, and a light chain constant region comprising amino acid residues 131 to 237 of SEQ ID NO: 217, or
iv) a heavy chain constant region comprising amino acid residues 147 to 475 of SEQ ID NO: 237, and a light chain constant region comprising amino acid residues 131 to 237 of SEQ ID NO: 217, and
the antibody that binds to human CD3 and cynomolgus monkey CD3 or antigen-binding fragment of the antibody comprises
i) a heavy chain constant region comprising amino acid residues 147 to 471 of SEQ ID NO: 207, and a light chain constant region comprising amino acid residues 133 to 238 of SEQ ID NO: 209, or
ii) a heavy chain constant region comprising amino acid residues 143 to 471 of SEQ ID NO: 211, and a light chain constant region comprising amino acid residues 131 to 236 of SEQ ID NO: 213.

61. The molecule according to claim 55, wherein the antibody that binds to human GPRC5D or an antigen-binding fragment of the antibody comprises a heavy chain variable region comprising amino acid residues 20 to 142 of SEQ ID NO: 76 and a light chain variable region comprising amino acid residues 21 to 127 of SEQ ID NO: 72, and comprises
i) a heavy chain constant region comprising by amino acid residues 147 to 475 of SEQ ID NO: 215, and a light chain constant region comprising amino acid residues 131 to 237 of SEQ ID NO: 217, or
ii) a heavy chain constant region comprising amino acid residues 147 to 475 of SEQ ID NO: 237, and a light chain constant region comprising amino acid residues 131 to 237 of SEQ ID NO: 217, and
the antibody that binds to human CD3 and cynomolgus monkey CD3 or antigen-binding fragment of the antibody comprises
i) a heavy chain constant region comprising amino acid residues 147 to 471 of SEQ ID NO: 207, and a light chain constant region comprising amino acid residues 133 to 238 of SEQ ID NO: 209, or
ii) a heavy chain constant region comprising amino acid residues 143 to 471 of SEQ ID NO: 211, and a light chain constant region comprising amino acid residues 131 to 236 of SEQ ID NO: 213.

62. The molecule according to claim 55, wherein
the antibody that binds to human GPRC5D or an antigen-binding fragment of the antibody comprises a heavy chain comprising amino acid residues 24 to 475 of SEQ ID NO: 119 and a light chain comprising amino acid residues 24 to 237 of SEQ ID NO: 203, and
the antibody that binds to human CD3 and cynomolgus monkey CD3 or antigen-binding fragment of the antibody comprises a heavy chain comprising amino acid residues 25 to 471 of SEQ ID NO: 207 and a light chain comprising amino acid residues 24 to 238 of SEQ ID NO: 209 or
the antibody that binds to human GPRC5D or an antigen-binding fragment of the antibody comprises a heavy chain comprising amino acid residues 24 to 475 of SEQ ID NO: 201 and a light chain comprising amino acid residues 24 to 237 of SEQ ID NO: 205, and
the antibody that binds to human CD3 and cynomolgus monkey CD3 or antigen-binding fragment of the antibody comprises a heavy chain comprising amino acid residues 25 to 471 of SEQ ID NO: 211 and a light chain comprising amino acid residues 24 to 236 of SEQ ID NO: 213.

63. The molecule according to claim 55, wherein the antibody that binds to human GPRC5D or an antigen-binding fragment of the antibody comprises a heavy chain variable region comprising amino acid residues 20 to 142 of SEQ ID NO: 76 and a light chain variable region comprising amino acid residues 21 to 127 of SEQ ID NO: 72, and mutated Fc, and
the antibody that binds to human CD3 and cynomolgus monkey CD3 or antigen-binding fragment of the antibody comprises mutated Fc.

64. The molecule according to claim 55, wherein
the antibody that binds to human GPRC5D or an antigen-binding fragment of the antibody comprises amino acid sequence represented by amino acid residues 24 to 271 of SEQ ID NO: 223 and mutated Fc, and
the antibody that binds to human CD3 and cynomolgus monkey CD3 or antigen-binding fragment of the antibody comprises amino acid residues 24 to 266 of SEQ ID NO: 219 and mutated Fc, or
the antibody that binds to human GPRC5D or an antigen-binding fragment of the antibody comprises amino acid residues 24 to 271 of SEQ ID NO: 223 and mutated Fc, and
the antibody that binds to human CD3 and cynomolgus monkey CD3 or antigen-binding fragment of the antibody comprises amino acid residues 24 to 264 of SEQ ID NO: 221 and mutated Fc.

65. The molecule according to claim 53, wherein the antibody that binds to human CD3 and cynomolgus monkey CD3 or antigen-binding fragment of the antibody is bound with the antibody that binds to human GPRC5D or antigen-binding fragment of the antibody via a linker or without a linker, wherein the antibody that binds to human GPRC5D or the antigen-binding fragment of the antibody comprises any one combination of:
a heavy chain variable region comprising SEQ ID NO: 97, and a light chain variable region comprising SEQ ID NO: 99,
a heavy chain variable region comprising SEQ ID NO: 101, and a light chain variable region comprising SEQ ID NO: 103,
a heavy chain variable region comprising SEQ ID NO: 105, and a light chain variable region comprising SEQ ID NO: 107, and
a heavy chain variable region comprising SEQ ID NO: 109, and a light chain variable region comprising SEQ ID NO: 135.

66. The molecule according to claim 54, wherein the molecule comprises any one of SEQ ID NOs: 171 to 179 and binds to human CD3 and cynomolgus monkey CD3 and to human GPRC5D.

67. A molecule which comprises an amino acid sequence encoded by a nucleotide sequence contained in a polynucleotide hybridizing under stringent conditions to a complementary strand of a polynucleotide comprising a nucleotide sequence encoding an amino acid sequence contained in an antibody that binds to human CD3 and cynomolgus monkey CD3 or an antigen-binding fragment of the antibody contained in a molecule according to claim 50, and binds to human CD3 and cynomolgus monkey CD3 and to human GPRC5D.

68. A molecule which comprises an amino acid sequence 90% or more identical to an amino acid sequence contained in an antibody that binds to human CD3 and cynomolgus monkey CD3 or an antigen-binding fragment of the antibody according to claim 50, and binds to human CD3 and cynomolgus monkey CD3 and to human GPRC5D.

69. A molecule which comprises an amino acid sequence derived by the substitution, deletion, or addition of 1 to several amino acid(s) from an amino acid sequence contained in an antibody that binds to human CD3 and cynomolgus monkey CD3 or an antigen-binding fragment of the antibody contained in a molecule according to claim 50, and binds to human CD3 and cynomolgus monkey CD3 and to human GPRC5D.

70. The molecule according to claim 50, wherein the molecule binds to cynomolgus monkey GPRC5D.

71. The molecule according to claim 43, wherein the molecule is bispecific.

72. The molecule according to claim 42, wherein the molecule is a polypeptide.

73. A polynucleotide comprising a nucleotide sequence encoding the amino acid sequence of the molecule according to claim 72.

74. A vector comprising the polynucleotide according to claim 73.

75. A cell comprising the polynucleotide according to claim 73.

76. A method for producing a molecule binding to human CD3 and cynomolgus monkey CD3 and to human GPRC5D, comprising the steps of: culturing a cell according to claim 75; and recovering a molecule binding to human CD3 and cynomolgus monkey CD3 and/or to human GPRC5D from the cultures.

77. A molecule binding to human CD3 and cynomolgus monkey CD3 and to human GPRC5D, the molecule being obtained by a method according to claim 76.

78. The molecule according to claim 77, wherein the molecule binds to cynomolgus monkey GPRC5D.

79. A pharmaceutical composition for treatment and/or prevention comprising a molecule according to claim 42 as an active ingredient and a pharmaceutically acceptable carrier.

80. A method of treating cancer comprising administering to a subject in need thereof the pharmaceutical composition according to claim 79.

81. The method according to claim 80, wherein the cancer is breast cancer, endometrial cancer, ovary cancer, lung cancer, stomach cancer, prostate cancer, kidney cancer, liver cancer, pancreatic cancer, colorectal cancer, esophageal cancer, urinary bladder cancer, uterine cervix cancer, blood cancer, lymphoma, or malignant melanoma expressing a GPRC5D protein.

82. The method according to claim 79, wherein the cancer is multiple myeloma expressing a GPRC5D protein.

83. A method for treating a cancer, comprising administering a molecule according to claim 42 to a subject in need thereof.

84. The pharmaceutical composition according to claim 79, wherein the pharmaceutical composition induces cytotoxicity to cells expressing GPRC5D by the redirection of T cells to the cells.

85. The method according to claim 83, wherein the method induces cytotoxicity to cells expressing GPRC5D by the redirection of T cells to the cells.

86. A method for inducing cytotoxicity to cells expressing GPRC5D by the redirection of T cells to the cells, comprising the step of administering to a subject in need thereof a molecule according to claim 42.

87. A method for redirecting T cells to cells expressing GPRC5D, comprising the step of administering to a subject in need thereof a molecule according to claim 42.


SEQUENCE LISTING (255 sequences)

SEQ ID NO: 1 - protein, 27 aa, Homo sapiens
  MISC_FEATURE (27)..(27): (27)   Xaa is biotinylated Glutamic Acid
MYKDCIESTGDYFLLCDAEGPWGIILX

SEQ ID NO: 2 - protein, 28 aa, Homo sapiens
  misc_feature (28)..(28): (28)    Xaa is biotinylated Lysine
MYKDCIESTGDYFLLCDAEGPWGIILEX

SEQ ID NO: 3 - DNA, 29 bp, Artificial Sequence
  primer for amplifying cDNA encording 2A4 VH by PCR
ctccagagttccaggtcacggtgactggc

SEQ ID NO: 4 - DNA, 366 bp, Rattus norvegicus
caggtccagttgcagcaatctggagctgagctggctaaacctgggacttcagtgaagctgtcctgcaaggcttctggctataccttcaccagctactatatttactgggtaaagcagaggcctggacagggccttgagtggatcggatatgtttatcctggatatggtggtacttactacagtgataagttcaagggcaaagccacatttactgcagacacatcctccagcacagcctacatgctactgggcagcctgacacctgaggactctgcgtactatttctgtgcaagacggaagggtataattcggggtccggggtactttgattactggggccaaggagtcatggtcacagtctcctca

SEQ ID NO: 5 - protein, 122 aa, Rattus norvegicus
QVQLQQSGAELAKPGTSVKLSCKASGYTFTSYYIYWVKQRPGQGLEWIGYVYPGYGGTYYSDKFKGKATFTADTSSSTAYMLLGSLTPEDSAYYFCARRKGIIRGPGYFDYWGQGVMVTVSS

SEQ ID NO: 6 - DNA, 369 bp, Rattus norvegicus
caggttactctgaaagagtctggccctgggatattgcagccctcccagaccctcagtctgacttgcactttctctgggttttcactgaacacttatgatatgggtgtgggctggattcgtcagccttcagggaagggtctggagtggctggcaaacatttggtgggatgatgataagtactacaatccatctctgagaaaccggctcacaatctccaaggacacctccaacaaccaagcattcctcaagatcaccaatgtggacactgcagatactgccacatactactgtgctcggatcgaaactgtccgggtttcccgtaaggggtttgctcactggggccaaggcactctggtcactgtctcttca

SEQ ID NO: 7 - protein, 123 aa, Rattus norvegicus
QVTLKESGPGILQPSQTLSLTCTFSGFSLNTYDMGVGWIRQPSGKGLEWLANIWWDDDKYYNPSLRNRLTISKDTSNNQAFLKITNVDTADTATYYCARIETVRVSRKGFAHWGQGTLVTVSS

SEQ ID NO: 8 - DNA, 369 bp, Rattus norvegicus
gagatacacctgcaggagtcaggacctggccttgtgaaaccttcacagtcactctccctcacctgttctgtcactggttacaccattaccagtggttatgattggagctggatccggaagttcccaggaaataaaatggagtggatggcatacatgagctatagaggtagcactaactacaacccctcgctcaaaagtcgaatctccattacaagagacacatccaagaatcagttcttcctgcagttgaattctgtcactactgaggatacagccacatattactgtgccctaacaaggacctattggtataactattactatgttttggatgcctggggtcaaggagcttcagtcactgtctcctca

SEQ ID NO: 9 - protein, 123 aa, Rattus norvegicus
EIHLQESGPGLVKPSQSLSLTCSVTGYTITSGYDWSWIRKFPGNKMEWMAYMSYRGSTNYNPSLKSRISITRDTSKNQFFLQLNSVTTEDTATYYCALTRTYWYNYYYVLDAWGQGASVTVSS

SEQ ID NO: 10 - DNA, 28 bp, Artificial Sequence
  primer for amplifying cDNA of 2A4_VH by PCR
tcagtaacactgtccaggacaccatctc

SEQ ID NO: 11 - DNA, 321 bp, Rattus norvegicus
gacatccagatgacacagtctccagcttccctgtctgcatctctgggagaaactgtctccatcgaatgtcttgcaagtgagggcatttccaatagtttagcgtggtatcagcagaagccagggaaatctcctcagctcctgatctatggtgcaagtagcttgcaagacggggtcccatcacggttcagtggcagtggttctggcacgcagtattctctcaagatcagcggcatgcaacctgaagatgaagggctttattactgtcaacagggttacaagtatcctccgacgttcggtggaggcaccaagctggaattgaaa

SEQ ID NO: 12 - protein, 107 aa, Rattus norvegicus
DIQMTQSPASLSASLGETVSIECLASEGISNSLAWYQQKPGKSPQLLIYGASSLQDGVPSRFSGSGSGTQYSLKISGMQPEDEGLYYCQQGYKYPPTFGGGTKLELK

SEQ ID NO: 13 - DNA, 321 bp, Rattus norvegicus
gaaactgtgatgacccagtctcccacatccatgtccacatcaataggagagagggtcaccctgaactgcaaggccagtcagagtgtgggtattaatgtagactggtaccaacagacaccagggcagtctcctaaactgctgatatatggggcatccaaccggcacactggggtccctgatcgcttcacaggcagtggatttgggagagatttcactctcaccatcagcaacgtggaggctgaagacctggctgtttattactgtctgcagcatggctccattcctccgacgttcggtggaggcaccaagctggaattgaaa

SEQ ID NO: 14 - protein, 107 aa, Rattus norvegicus
ETVMTQSPTSMSTSIGERVTLNCKASQSVGINVDWYQQTPGQSPKLLIYGASNRHTGVPDRFTGSGFGRDFTLTISNVEAEDLAVYYCLQHGSIPPTFGGGTKLELK

SEQ ID NO: 15 - DNA, 318 bp, Rattus norvegicus
gacatccagatgacccagtctccttcattcctgtctgcatctgtgggagaaagagtcactctcagctgcaaagcaagtcagaatattaacaagtacttagactggtatcagcaaaagcttggagaacctcccaaactcctgatatataatacaaacaatttgcatacgggcatcccatcaaggttcagtggcagtggatctggtacagattacacactcaccatcagcggcctgcagcctgaagatgttgccacatatttctgcttgcagcgtaatagttggtacacgtttggagctgggaccaagctggaactgaaa

SEQ ID NO: 16 - protein, 106 aa, Rattus norvegicus
DIQMTQSPSFLSASVGERVTLSCKASQNINKYLDWYQQKLGEPPKLLIYNTNNLHTGIPSRFSGSGSGTDYTLTISGLQPEDVATYFCLQRNSWYTFGAGTKLELK

SEQ ID NO: 17 - DNA, 449 bp, Artificial Sequence
  DNA fragment comprising DNA sequence encording amino acid sequence of human k-chain of secretion signal sequence and human k-chain of constant region
gcctccggactctagagccaccatggtgctgcagacccaggtgttcatctccctgctgctgtggatctccggcgcgtacggcgatatcgtgatgattaaacgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgttaggggcccgtttaaacgggggaggcta

SEQ ID NO: 18 - DNA, 30 bp, Artificial Sequence
  primer F for expression vector of VL
tataccgtcgacctctagctagagcttggc

SEQ ID NO: 19 - DNA, 30 bp, Artificial Sequence
  primer R for expression vector of VL
gctatggcagggcctgccgccccgacgttg

SEQ ID NO: 20 - DNA, 1132 bp, Artificial Sequence
  DNA fragment comprising DNA sequence encording amino acid sequence of human heavy chain of signal sequence and human IgG1 constant region
gcctccggactctagagccaccatgaaacacctgtggttcttcctcctgctggtggcagctcccagatgggtgctgagccaggtgcaattgtgcaggcggttagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaatgagatatcgggcccgtttaaacgggggaggcta

SEQ ID NO: 21 - DNA, 702 bp, Artificial Sequence
  nucleotide sequence of c2A4  L
atggtgctgcagacccaggtgttcatctccctgctgctgtggatctccggcgcgtacggcgacatccagatgacacagtctccagcttccctgtctgcatctctgggagaaactgtctccatcgaatgtcttgcaagtgagggcatttccaatagtttagcgtggtatcagcagaagccagggaaatctcctcagctcctgatctatggtgcaagtagcttgcaagacggggtcccatcacggttcagtggcagtggttctggcacgcagtattctctcaagatcagcggcatgcaacctgaagatgaagggctttattactgtcaacagggttacaagtatcctccgacgttcggtggaggcaccaagctggaattgaaacgggctgtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 22 - protein, 234 aa, Artificial Sequence
  amino acid sequence of c2A4 L
MVLQTQVFISLLLWISGAYGDIQMTQSPASLSASLGETVSIECLASEGISNSLAWYQQKPGKSPQLLIYGASSLQDGVPSRFSGSGSGTQYSLKISGMQPEDEGLYYCQQGYKYPPTFGGGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 44 bp, Artificial Sequence
  primer set F for c2A4 L
atctccggcgcgtacggcgacatccagatgacacagtctccagc

SEQ ID NO: 24 - DNA, 44 bp, Artificial Sequence
  primer set R for c2A4 L
ggagggggcggccacagcccgtttcaattccagcttggtgcctc

SEQ ID NO: 25 - DNA, 1413 bp, Artificial Sequence
  nucleotide sequence of c2A4 H
atgaaacacctgtggttcttcctcctgctggtggcagctcccagatgggtgctgagccaggtccagttgcagcaatctggagctgagctggctaaacctgggacttcagtgaagctgtcctgcaaggcttctggctataccttcaccagctactatatttactgggtaaagcagaggcctggacagggccttgagtggatcggatatgtttatcctggatatggtggtacttactacagtgataagttcaagggcaaagccacatttactgcagacacatcctccagcacagcctacatgctactgggcagcctgacacctgaggactctgcgtactatttctgtgcaagacggaagggtataattcggggtccggggtactttgattactggggccaaggagtcatggtcacagtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 26 - protein, 471 aa, Artificial Sequence
  amino acid sequence of c2A4 H
MKHLWFFLLLVAAPRWVLSQVQLQQSGAELAKPGTSVKLSCKASGYTFTSYYIYWVKQRPGQGLEWIGYVYPGYGGTYYSDKFKGKATFTADTSSSTAYMLLGSLTPEDSAYYFCARRKGIIRGPGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 45 bp, Artificial Sequence
  primer set F for c2A4 H
ccagatgggtgctgagccaggtccagttgcagcaatctggagctg

SEQ ID NO: 28 - DNA, 46 bp, Artificial Sequence
  primer set R for c2A4 H
cttggtggaggctgagctgactgtgaccatgactccttggccccag

SEQ ID NO: 29 - DNA, 702 bp, Artificial Sequence
  nucleotide sequence of c2B1 L
atggtgctgcagacccaggtgttcatctccctgctgctgtggatctccggcgcgtacggcgaaactgtgatgacccagtctcccacatccatgtccacatcaataggagagagggtcaccctgaactgcaaggccagtcagagtgtgggtattaatgtagactggtaccaacagacaccagggcagtctcctaaactgctgatatatggggcatccaaccggcacactggggtccctgatcgcttcacaggcagtggatttgggagagatttcactctcaccatcagcaacgtggaggctgaagacctggctgtttattactgtctgcagcatggctccattcctccgacgttcggtggaggcaccaagctggaattgaaacgggctgtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 30 - protein, 234 aa, Artificial Sequence
  amino acid sequence of c2B1 L
MVLQTQVFISLLLWISGAYGETVMTQSPTSMSTSIGERVTLNCKASQSVGINVDWYQQTPGQSPKLLIYGASNRHTGVPDRFTGSGFGRDFTLTISNVEAEDLAVYYCLQHGSIPPTFGGGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 44 bp, Artificial Sequence
  primer set F for c2B1 L
atctccggcgcgtacggcgaaactgtgatgacccagtctcccac

SEQ ID NO: 32 - DNA, 44 bp, Artificial Sequence; identical to SEQ ID NO: 24
  primer set R for c2B1 L

SEQ ID NO: 33 - DNA, 1416 bp, Artificial Sequence
  nucleotide sequence of  c2B1 H
atgaaacacctgtggttcttcctcctgctggtggcagctcccagatgggtgctgagccaggttactctgaaagagtctggccctgggatattgcagccctcccagaccctcagtctgacttgcactttctctgggttttcactgaacacttatgatatgggtgtgggctggattcgtcagccttcagggaagggtctggagtggctggcaaacatttggtgggatgatgataagtactacaatccatctctgagaaaccggctcacaatctccaaggacacctccaacaaccaagcattcctcaagatcaccaatgtggacactgcagatactgccacatactactgtgctcggatcgaaactgtccgggtttcccgtaaggggtttgctcactggggccaaggcactctggtcactgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 34 - protein, 472 aa, Artificial Sequence
  amino acid sequence of c2B1 H
MKHLWFFLLLVAAPRWVLSQVTLKESGPGILQPSQTLSLTCTFSGFSLNTYDMGVGWIRQPSGKGLEWLANIWWDDDKYYNPSLRNRLTISKDTSNNQAFLKITNVDTADTATYYCARIETVRVSRKGFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 45 bp, Artificial Sequence
  primer set F for c2B1 H
ccagatgggtgctgagccaggttactctgaaagagtctggccctg

SEQ ID NO: 36 - DNA, 46 bp, Artificial Sequence
  primer set R for c2B1 H
cttggtggaggctgagctgacagtgaccagagtgccttggccccag

SEQ ID NO: 37 - DNA, 699 bp, Artificial Sequence
  nucleotide sequence of c7B4 L
atggtgctgcagacccaggtgttcatctccctgctgctgtggatctccggcgcgtacggcgacatccagatgacccagtctccttcattcctgtctgcatctgtgggagaaagagtcactctcagctgcaaagcaagtcagaatattaacaagtacttagactggtatcagcaaaagcttggagaacctcccaaactcctgatatataatacaaacaatttgcatacgggcatcccatcaaggttcagtggcagtggatctggtacagattacacactcaccatcagcggcctgcagcctgaagatgttgccacatatttctgcttgcagcgtaatagttggtacacgtttggagctgggaccaagctggaactgaaacgggctgtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 38 - protein, 233 aa, Artificial Sequence
  amino acid sequence of c7B4 L
MVLQTQVFISLLLWISGAYGDIQMTQSPSFLSASVGERVTLSCKASQNINKYLDWYQQKLGEPPKLLIYNTNNLHTGIPSRFSGSGSGTDYTLTISGLQPEDVATYFCLQRNSWYTFGAGTKLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 44 bp, Artificial Sequence
  primer set F for c7B4 L
atctccggcgcgtacggcgacatccagatgacccagtctccttc

SEQ ID NO: 40 - DNA, 44 bp, Artificial Sequence
  primer set R for c7B4 L
ggagggggcggccacagcccgtttcagttccagcttggtcccag

SEQ ID NO: 41 - DNA, 1416 bp, Artificial Sequence
  nucleotide sequence of c7B4 H
atgaaacacctgtggttcttcctcctgctggtggcagctcccagatgggtgctgagcgagatacacctgcaggagtcaggacctggccttgtgaaaccttcacagtcactctccctcacctgttctgtcactggttacaccattaccagtggttatgattggagctggatccggaagttcccaggaaataaaatggagtggatggcatacatgagctatagaggtagcactaactacaacccctcgctcaaaagtcgaatctccattacaagagacacatccaagaatcagttcttcctgcagttgaattctgtcactactgaggatacagccacatattactgtgccctaacaaggacctattggtataactattactatgttttggatgcctggggtcaaggagcttcagtcactgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 42 - protein, 472 aa, Artificial Sequence
  amino acid sequence of c7B4 H
MKHLWFFLLLVAAPRWVLSEIHLQESGPGLVKPSQSLSLTCSVTGYTITSGYDWSWIRKFPGNKMEWMAYMSYRGSTNYNPSLKSRISITRDTSKNQFFLQLNSVTTEDTATYYCALTRTYWYNYYYVLDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 45 bp, Artificial Sequence
  primer set F for c7B4 H
ccagatgggtgctgagcgagatacacctgcaggagtcaggacctg

SEQ ID NO: 44 - DNA, 46 bp, Artificial Sequence
  primer set R for c7B4 H
cttggtggaggctgagctgacagtgactgaagctccttgaccccag

SEQ ID NO: 45 - protein, 8 aa, Artificial Sequence
  amino acid sequence of 2A4 H CDR1
GYTFTSYY

SEQ ID NO: 46 - protein, 8 aa, Artificial Sequence
  amino acid sequence of 2A4 H CDR2
VYPGYGGT

SEQ ID NO: 47 - protein, 15 aa, Artificial Sequence
  amino acid sequence of 2A4 H CDR3
ARRKGIIRGPGYFDY

SEQ ID NO: 48 - protein, 10 aa, Artificial Sequence
  amino acid sequence of 2B1 H CDR1
GFSLNTYDMG

SEQ ID NO: 49 - protein, 7 aa, Artificial Sequence
  amino acid sequence of 2B1 H CDR2
IWWDDDK

SEQ ID NO: 50 - protein, 15 aa, Artificial Sequence
  amino acid sequence of 2B1 H CDR3
ARIETVRVSRKGFAH

SEQ ID NO: 51 - protein, 9 aa, Artificial Sequence
  amino acid sequence of 7B4 H CDR1
GYTITSGYD

SEQ ID NO: 52 - protein, 7 aa, Artificial Sequence
  amino acid sequence of 7B4 H CDR2
MSYRGST

SEQ ID NO: 53 - protein, 16 aa, Artificial Sequence
  amino acid sequence of 7B4 H CDR3
ALTRTYWYNYYYVLDA

SEQ ID NO: 54 - protein, 6 aa, Artificial Sequence
  amino acid sequence of 2A4 L CDR1
EGISNS

SEQ ID NO: 55 - protein, 3 aa, Artificial Sequence
  amino acid sequence of 2A4 L CDR2
GAS

SEQ ID NO: 56 - protein, 9 aa, Artificial Sequence
  amino acid sequence of 2A4 L CDR3
QQGYKYPPT

SEQ ID NO: 57 - protein, 6 aa, Artificial Sequence
  amino acid sequence of 2B1 L CDR1
QSVGIN

SEQ ID NO: 58 - protein, 3 aa, Artificial Sequence
  amino acid sequence of 2B1 L CDR2
GAS

SEQ ID NO: 59 - protein, 9 aa, Artificial Sequence
  amino acid sequence of 2B1 L CDR3
LQHGSIPPT

SEQ ID NO: 60 - protein, 6 aa, Artificial Sequence
  amino acid sequence of 7B4 L CDR1
QNINKY

SEQ ID NO: 61 - protein, 3 aa, Artificial Sequence
  amino acid sequence of 7B4 L CDR2
NTN

SEQ ID NO: 62 - protein, 8 aa, Artificial Sequence
  amino acid sequence of 7B4 L CDR3
LQRNSWYT

SEQ ID NO: 63 - DNA, 702 bp, Artificial Sequence
  nucleotide sequence of h2B1_L1
atggtgctgcagacccaggtgttcatctccctgctgctgtggatctccggcgcgtacggcgataccgtgatgacccagagccctgacagcctggccgtgtctctgggagagagagccaccatcaactgcaaggccagccagagcgtgggcatcaacgtggactggtatcagcagaagcccggccagccccccaagctgctgatctacggcgccagcaatagacacaccggcgtgcccgatagattcagcggctctggcagcggcagagacttcaccctgaccatcagctccctgcaggccgaggatgtggccgtgtactactgtctgcagcacggcagcatcccccccacatttggccagggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 64 - protein, 234 aa, Artificial Sequence
  amino acid sequence of h2B1_L1
MVLQTQVFISLLLWISGAYGDTVMTQSPDSLAVSLGERATINCKASQSVGINVDWYQQKPGQPPKLLIYGASNRHTGVPDRFSGSGSGRDFTLTISSLQAEDVAVYYCLQHGSIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - DNA, 702 bp, Artificial Sequence
  nucleotide sequence of h2B1_L2
atggtgctgcagacccaggtgttcatctccctgctgctgtggatctccggcgcgtacggcgataccgtgatgacccagagccctgacagcctggccgtgtctctgggagagagagccaccatcaactgcaaggccagccagagcgtgggcatcaacgtggactggtatcagcagaagcccggccagccccccaagctgctgatctacggcgccagcaatagacacaccggcgtgcccgatagattcagcggcagcggcttcggcaccgacttcaccctgacaatcagctccctgcaggccgaggacgtggccgtgtactactgtctgcagcacggcagcatcccccccacatttggccagggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 66 - protein, 234 aa, Artificial Sequence
  amino acid sequence of h2B1_L2
MVLQTQVFISLLLWISGAYGDTVMTQSPDSLAVSLGERATINCKASQSVGINVDWYQQKPGQPPKLLIYGASNRHTGVPDRFSGSGFGTDFTLTISSLQAEDVAVYYCLQHGSIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 702 bp, Artificial Sequence
  nucleotide sequence of h2B1_L3
atggtgctgcagacccaggtgttcatctccctgctgctgtggatctccggcgcgtacggcgataccgtgatgacccagagccctgacagcctggccgtgtctctgggagagagagccaccatcaactgcaaggccagccagagcgtgggcatcaacgtggactggtatcagcagaagcccggccagccccccaagctgctgatctacggcgccagcaatagacacaccggcgtgcccgatagattcagcggcagcggcttcggcagagacttcaccctgaccatcagctccctgcaggccgaggatgtggccgtgtactactgtctgcagcacggcagcatcccccccacatttggccagggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 68 - protein, 234 aa, Artificial Sequence
  amino acid sequence of h2B1_L3
MVLQTQVFISLLLWISGAYGDTVMTQSPDSLAVSLGERATINCKASQSVGINVDWYQQKPGQPPKLLIYGASNRHTGVPDRFSGSGFGRDFTLTISSLQAEDVAVYYCLQHGSIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - DNA, 702 bp, Artificial Sequence
  nucleotide sequence of h2B1_L4
atggtgctgcagacccaggtgttcatctccctgctgctgtggatctccggcgcgtacggcgataccgtgatgacccagagccctgacagcctggccgtgtctctgggagagagagccaccatcaactgcaaggccagccagagcgtgggcatcaacgtggactggtatcagcagaagcccggccagagccccaagctgctgatctacggcgccagcaacagacacaccggcgtgcccgatagattcagcggcagcggcttcggcagagacttcaccctgaccatcagctccctgcaggccgaggatgtggccgtgtactactgtctgcagcacggcagcatcccccccacatttggccagggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 70 - protein, 234 aa, Artificial Sequence
  amino acid sequence of h2B1_L4
MVLQTQVFISLLLWISGAYGDTVMTQSPDSLAVSLGERATINCKASQSVGINVDWYQQKPGQSPKLLIYGASNRHTGVPDRFSGSGFGRDFTLTISSLQAEDVAVYYCLQHGSIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 702 bp, Artificial Sequence
  nucleotide sequence of h2B1_L5
atggtgctgcagacccaggtgttcatctccctgctgctgtggatctccggcgcgtacggcgccaccagaatgacacagagccccagcagcttcagcgccagcaccggcgacagagtgaccatcacatgcaaggccagccagagcgtgggcatcaacgtggactggtatcagcagaagcccggcaagagccccaagctgctgatctacggcgcctccaacagacacaccggcgtgcccagcagattttccggcagcggcttcggcagagacttcaccctgaccatcagcagcctgcagagcgaggacttcgccacctactactgcctgcagcacggcagcatcccccctacatttggcggaggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 72 - protein, 234 aa, Artificial Sequence
  amino acid sequence of h2B1_L5
MVLQTQVFISLLLWISGAYGATRMTQSPSSFSASTGDRVTITCKASQSVGINVDWYQQKPGKSPKLLIYGASNRHTGVPSRFSGSGFGRDFTLTISSLQSEDFATYYCLQHGSIPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 1416 bp, Artificial Sequence
  nucleotide sequence of h2B1_H1
atgaaacacctgtggttcttcctcctgctggtggcagctcccagatgggtgctgagccaggtgcagctggtggaatctggcggcggactcgtgaagcctagccagaccctgagcctgacctgcaccttcagcggcttcagcctgaacacctacgacatgggcgtgggctggatcagacagcctcctggcaagggcctggaatggatcggcaacatttggtgggacgacgacaagtactacaaccccagcctgaagtccagagtgaccatcagcgtggacaccagcaagaaccaggcctccctgaagctgagcagcgtgacagccgccgataccgccgtgtactactgcgccagaatcgagacagtgcgggtgtccagaaagggcttcgcccactggggacagggcacactcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 74 - protein, 472 aa, Artificial Sequence
  amino acid sequence of h2B1_H1
MKHLWFFLLLVAAPRWVLSQVQLVESGGGLVKPSQTLSLTCTFSGFSLNTYDMGVGWIRQPPGKGLEWIGNIWWDDDKYYNPSLKSRVTISVDTSKNQASLKLSSVTAADTAVYYCARIETVRVSRKGFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 1416 bp, Artificial Sequence
  nucleotide sequence of h2B1_H2
atgaaacacctgtggttcttcctcctgctggtggcagctcccagatgggtgctgagccaggtgcagctggtggaatctggcggcggactcgtgaagcctagccagaccctgagcctgacctgcaccttcagcggcttcagcctgaacacctacgacatgggcgtgggctggatcagacagcctcctggcaagggcctggaatggatcggcaacatttggtgggacgacgacaagtactacaaccccagcctgaagtccagagtgaccatcagcaaggacaccagcaacaaccaggcctccctgaagctgagcagcgtgacagccgccgataccgccgtgtactactgcgccagaatcgagacagtgcgggtgtccagaaagggcttcgcccactggggacagggcacactcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 76 - protein, 472 aa, Artificial Sequence
  amino acid sequence of h2B1_H2
MKHLWFFLLLVAAPRWVLSQVQLVESGGGLVKPSQTLSLTCTFSGFSLNTYDMGVGWIRQPPGKGLEWIGNIWWDDDKYYNPSLKSRVTISKDTSNNQASLKLSSVTAADTAVYYCARIETVRVSRKGFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - DNA, 1416 bp, Artificial Sequence
  nucleotide sequence of h2B1_H3
atgaaacacctgtggttcttcctcctgctggtggcagctcccagatgggtgctgagccaggtgcagctggtggaatctggcggcggactcgtgaagcctagccagaccctgagcctgacctgcaccttcagcggcttcagcctgaacacctacgacatgggcgtgggctggatcagacagcctcctggcaagggcctggaatggatcgccaacatttggtgggacgacgacaagtactacaaccccagcctgaagtccagagtgaccatcagcaaggacaccagcaacaaccaggcctccctgaagctgagcagcgtgacagccgccgataccgccgtgtactactgcgccagaatcgagacagtgcgggtgtccagaaagggcttcgcccactggggacagggcacactcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 78 - protein, 472 aa, Artificial Sequence
  amino acid sequence of h2B1_H3
MKHLWFFLLLVAAPRWVLSQVQLVESGGGLVKPSQTLSLTCTFSGFSLNTYDMGVGWIRQPPGKGLEWIANIWWDDDKYYNPSLKSRVTISKDTSNNQASLKLSSVTAADTAVYYCARIETVRVSRKGFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - DNA, 1416 bp, Artificial Sequence
  nucleotide sequence of h2B1_H4
atgaaacacctgtggttcttcctcctgctggtggcagctcccagatgggtgctgagccaagtgaccctgaaagagtccggccctgccctcgtgaagcctacccagaccctgacactgacctgcaccttcagcggcttcagcctgaacacctacgacatgggcgtgggctggatcagacagcctcctggcaagggcctggaatggctggccaacatttggtgggacgacgacaagtactacagccccagcctgaagtcccggctgaccatcaccaaggacaccagcaacaaccaggccgtgctgaccatgacaaacatggaccccgtggacaccgccacctactactgcgccagaatcgagacagtgcgggtgtccagaaagggcttcgcccactggggacagggcacactcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 80 - protein, 472 aa, Artificial Sequence
  amino acid sequence of h2B1_H4
MKHLWFFLLLVAAPRWVLSQVTLKESGPALVKPTQTLTLTCTFSGFSLNTYDMGVGWIRQPPGKGLEWLANIWWDDDKYYSPSLKSRLTITKDTSNNQAVLTMTNMDPVDTATYYCARIETVRVSRKGFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 699 bp, Artificial Sequence
  nucleotide sequence of h7B4_L1
atggtgctgcagacccaggtgttcatctccctgctgctgtggatctccggcgcgtacggcgagatcgtgctgacacagagccctggcaccctgagcctgtctccaggcgaaagagccaccctgtcctgcaaggccagccagaacatcaacaagtacctggactggtatcagcagaagcccggccagccccccagactgctgatctacaacaccaacaacctgcacaccggcatccccgacagattcagcggctctggcagcggcaccgactacaccctgaccatcagcagactggaacccgaggacttcgccgtgtactactgcctgcagcggaacagctggtacaccttcggccagggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 82 - protein, 233 aa, Artificial Sequence
  amino acid sequence of h7B4_L1
MVLQTQVFISLLLWISGAYGEIVLTQSPGTLSLSPGERATLSCKASQNINKYLDWYQQKPGQPPRLLIYNTNNLHTGIPDRFSGSGSGTDYTLTISRLEPEDFAVYYCLQRNSWYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - DNA, 699 bp, Artificial Sequence
  nucleotide sequence of h7B4_L2
atggtgctgcagacccaggtgttcatctccctgctgctgtggatctccggcgcgtacggcgacatccagatgacccagagccctagcagcctgagcctgtcccctggcgaaagagccaccctgagctgcaaggccagccagaacatcaacaagtacctggactggtatcagcagaagcccggccagccccccagactgctgatctacaacaccaacaacctgcacaccggcatccccgacagattcagcggctctggcagcggcaccgactacaccctgaccatcagcagactggaacccgaggacttcgccgtgtactactgcctgcagcggaacagctggtacaccttcggccagggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 84 - protein, 233 aa, Artificial Sequence
  amino acid sequence of h7B4_L2
MVLQTQVFISLLLWISGAYGDIQMTQSPSSLSLSPGERATLSCKASQNINKYLDWYQQKPGQPPRLLIYNTNNLHTGIPDRFSGSGSGTDYTLTISRLEPEDFAVYYCLQRNSWYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - DNA, 1416 bp, Artificial Sequence
  nucleotide sequence of h7B4_H1
atgaaacacctgtggttcttcctcctgctggtggcagctcccagatgggtgctgagccaggtgcagctgcaggaatctggccctggcctcgtgaagcctagccagaccctgagcctgacctgtaccgtgtccggctacaccatcaccagcggctacgactggtcctggatcagacagcctcctggcaagggcctggaatggatcgcctacatgagctaccggggcagcaccaactacaaccccagcctgaagtccagagtgaccatcagccgggacaccagcaagaaccagttctccctgaagctgagcagcgtgacagccgccgataccgccgtgtactactgcgccctgacccggacctactggtacaactactactacgtgctggacgcctggggccagggcacactcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 86 - protein, 472 aa, Artificial Sequence
  amino acid sequence of h7B4_H1
MKHLWFFLLLVAAPRWVLSQVQLQESGPGLVKPSQTLSLTCTVSGYTITSGYDWSWIRQPPGKGLEWIAYMSYRGSTNYNPSLKSRVTISRDTSKNQFSLKLSSVTAADTAVYYCALTRTYWYNYYYVLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 1416 bp, Artificial Sequence
  nucleotide sequence of h7B4_H2
atgaaacacctgtggttcttcctcctgctggtggcagctcccagatgggtgctgagcgagatccagctgcaggaatctggccctggcctcgtgaagcctagccagaccctgagcctgacctgtaccgtgtccggctacaccatcaccagcggctacgactggtcctggatcagacagcctcctggcaagggcctggaatggatcggctacatgagctaccggggcagcaccaactacaaccccagcctgaagtccagagtgaccatcagccgggacaccagcaagaaccagttctccctgaagctgagcagcgtgacagccgccgataccgccgtgtactactgcgccctgacccggacctactggtacaactactactacgtgctggacgcctggggccagggcacactcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 88 - protein, 472 aa, Artificial Sequence
  amino acid sequence of h7B4_H2
MKHLWFFLLLVAAPRWVLSEIQLQESGPGLVKPSQTLSLTCTVSGYTITSGYDWSWIRQPPGKGLEWIGYMSYRGSTNYNPSLKSRVTISRDTSKNQFSLKLSSVTAADTAVYYCALTRTYWYNYYYVLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 1416 bp, Artificial Sequence
  nucleotide sequence of h7B4_H3
atgaaacacctgtggttcttcctcctgctggtggcagctcccagatgggtgctgagcgagatccagctgcaggaatctggccctggcctcgtgaagcctagccagaccctgagcctgacctgtaccgtgtccggctacaccatcaccagcggctacgactggtcctggatcagacagcctcctggcaagggcctggaatggatcgcctacatgagctaccggggcagcaccaactacaaccccagcctgaagtccagagtgaccatcagccgggacaccagcaagaaccagttctccctgaagctgagcagcgtgacagccgccgataccgccgtgtactactgcgccctgacccggacctactggtacaactactactacgtgctggacgcctggggccagggcacactcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 90 - protein, 472 aa, Artificial Sequence
  amino acid sequence of h7B4_H3
MKHLWFFLLLVAAPRWVLSEIQLQESGPGLVKPSQTLSLTCTVSGYTITSGYDWSWIRQPPGKGLEWIAYMSYRGSTNYNPSLKSRVTISRDTSKNQFSLKLSSVTAADTAVYYCALTRTYWYNYYYVLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1416 bp, Artificial Sequence
  nucleotide sequence of h7B4_H5
atgaaacacctgtggttcttcctcctgctggtggcagctcccagatgggtgctgagcgagatccagctgcaggaatctggccctggcctcgtgaagcctagccagaccctgagcctgacctgtaccgtgtccggctacaccatcaccagcggctacgactggtcctggatcagaaagccccctggcaacaagatggaatggatcgcctacatgagctaccggggcagcaccaactacaaccccagcctgaagtccagagtgaccatcagccgggacaccagcaagaaccagttctccctgaagctgagcagcgtgacagccgccgataccgccgtgtactactgcgccctgacccggacctactggtacaactactactacgtgctggacgcctggggccagggcacactcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 92 - protein, 472 aa, Artificial Sequence
  amino acid sequence of h7B4_H5
MKHLWFFLLLVAAPRWVLSEIQLQESGPGLVKPSQTLSLTCTVSGYTITSGYDWSWIRKPPGNKMEWIAYMSYRGSTNYNPSLKSRVTISRDTSKNQFSLKLSSVTAADTAVYYCALTRTYWYNYYYVLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 28 aa, Artificial Sequence
  polypeptide encording N-terminal sequence of cynomologous GPRC5D
  misc_feature (28)..(28): 28   X is biotinylated Lysine
MYKDCIESTGDYFLPCDSEGPWGIVLEX

SEQ ID NO: 94 - DNA, 26 bp, Artificial Sequence
  primer A used for sequence analysis of scFv
ctcttcgctattacgccagctggcga

SEQ ID NO: 95 - DNA, 30 bp, Artificial Sequence
  primer B used for sewquence analysis of scFv
ataacaatttcacacaggaaacagctatga

SEQ ID NO: 96 - DNA, 345 bp, Artificial Sequence
  nucleotide sequence of C2037 VH
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagtgggaagagtaactacccctggggccagggaaccctggtcaccgtctcctca

SEQ ID NO: 97 - protein, 115 aa, Artificial Sequence
  amino acid sequence of C2037 VH
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCASGKSNYPWGQGTLVTVSS

SEQ ID NO: 98 - DNA, 330 bp, Artificial Sequence
  nucleotide sequence of C2037 VL
cagcctgtgctgactcagccaccctcagcgtctgggacccccgggcagagggtcaccatctcttgttctggaagcaactccaacatcggcagtagaactgtgaattggtaccagcacctcccaggaacggcccccagactcctcatctatggtaataatcagtggccctcaggggtccctgaccgattctctggctctaagtctggctcctcagcctccctggccatcagtgggctccggtccgaggatgaggctgattattactgtgcagcatgggatgacagcctgagtggtgtggtattcggcggagggaccaagctgaccgtccta

SEQ ID NO: 99 - protein, 110 aa, Artificial Sequence
  amino acid sequence of C2037 VL
QPVLTQPPSASGTPGQRVTISCSGSNSNIGSRTVNWYQHLPGTAPRLLIYGNNQWPSGVPDRFSGSKSGSSASLAISGLRSEDEADYYCAAWDDSLSGVVFGGGTKLTVL

SEQ ID NO: 100 - DNA, 369 bp, Artificial Sequence
  nucleotide sequence of C3048 VH
caggtgcagctggtgcagtctggggctgaggtgaagaagcctgggtcctcggtgaaggtctcctgcaaggcttctggaggcaccttcagcaggtatgctatcagctgggtgcgacaggcccctggacaagggcttgagtggatgggagggatcatccctatctttggaacagcaaactacgcacagaagttccagggcagagtcacaattaccgcggaccaatccacgagaacagcctacatggacctgggcagactgagatctgaggacacggccgtgtattattgtgctctcgagggattcagggactcattaaaacgaaatccttttgatatctggggccaagggacaatggtcaccgtctcttca

SEQ ID NO: 101 - protein, 123 aa, Artificial Sequence
  amino acid sequence of C3048 VH
QVQLVQSGAEVKKPGSSVKVSCKASGGTFSRYAISWVRQAPGQGLEWMGGIIPIFGTANYAQKFQGRVTITADQSTRTAYMDLGRLRSEDTAVYYCALEGFRDSLKRNPFDIWGQGTMVTVSS

SEQ ID NO: 102 - DNA, 330 bp, Artificial Sequence
  nucleotide sequence of C3048 VL
cagtctgtcgtgacgcagccgccctcagtgtctgcggccccaggacagaaggtcaccatctcctgctctggaggcaccaccaacattgggagtaattatgtatcctggtatcagcagctcccaggaacagcccccaaattcctcatgtatgaaaataataagcgaccctcaggcattcctgaccgattctctggctccaagtctggcacgtcagccaccctggccatcaccggactccagactggggacgaggccacttattactgctcaacatgggatagcagcctgaatactgggctattcggcggagggaccaagctgaccgtccta

SEQ ID NO: 103 - protein, 110 aa, Artificial Sequence
  amino acid sequence of C3048 VL
QSVVTQPPSVSAAPGQKVTISCSGGTTNIGSNYVSWYQQLPGTAPKFLMYENNKRPSGIPDRFSGSKSGTSATLAITGLQTGDEATYYCSTWDSSLNTGLFGGGTKLTVL

SEQ ID NO: 104 - DNA, 363 bp, Artificial Sequence
  nucleotide sequence of C3015 VH
gaggtgcagctggtggagtccgggggaggcgtggtccagcctgggaggtccctgagactctcctgtgcagcctctggattcacctttaggaactatgccatgagctgggtccgccaggctccagggaaggggctggagtgggtctcaggtattagtggtactggtggtagcacatactacgcagactccgtgaagggccggttcaccatctccagagacaattccaaggacacactatatctgcaattgaacagcctgagagccgaggacacggccgtatattactgtgcgaaatcccgtagtatgagtatcaactacggtatggacgtctggggccaagggaccacggtcaccgtctcctca

SEQ ID NO: 105 - protein, 121 aa, Artificial Sequence
  amino acid sequence of C3015 VH
EVQLVESGGGVVQPGRSLRLSCAASGFTFRNYAMSWVRQAPGKGLEWVSGISGTGGSTYYADSVKGRFTISRDNSKDTLYLQLNSLRAEDTAVYYCAKSRSMSINYGMDVWGQGTTVTVSS

SEQ ID NO: 106 - DNA, 318 bp, Artificial Sequence
  nucleotide sequence of C3015 VL
tcctatgagctgactcagccaccctcagtgtccgtgtccccaggacagacagtcagcatcacctgctctggagataatttgggtgatagatatgcctcctggtatcagcagaagccaggccagtcccctgtgcaggtcatctatcaagataccaagcggccctcagggatccctgagcgattctctggctccaactctgggaacacagccactctgaccatcagcgggacccaggctatggatgaggctgactattactgtcaggcgtgggacagcaacactgtggtattcggcggagggaccaagctgaccgtccta

SEQ ID NO: 107 - protein, 106 aa, Artificial Sequence
  amino acid sequence of C3015 VL
SYELTQPPSVSVSPGQTVSITCSGDNLGDRYASWYQQKPGQSPVQVIYQDTKRPSGIPERFSGSNSGNTATLTISGTQAMDEADYYCQAWDSNTVVFGGGTKLTVL

SEQ ID NO: 108 - DNA, 345 bp, Artificial Sequence
  nucleotide sequence of C3022 VH
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcgggctgagatctgacgacacggccgtttattactgtgcgagtggtagtgtaaggcatccctggggccagggaaccctggtcaccgtctcctca

SEQ ID NO: 109 - protein, 115 aa, Artificial Sequence
  amino acid sequence of C3022 VH
QVQLVQSGAEVKKPGASVKVSCKASGYTFTGYYMHWVRQAPGQGLEWMGRINPNSGGTNYAQKFQGRVTMTRDTSISTAYMELSGLRSDDTAVYYCASGSVRHPWGQGTLVTVSS

SEQ ID NO: 110 - DNA, 330 bp, Artificial Sequence
  nucleotide sequence of C3022 VL
cagcctgtgctgactcagccaccctcagcgtctgggacccccgggcagagggtcaccatctcttgttctggaagcaggtccaacatcggaggtaatattgtaagttggtaccagcagttcccaggaacggcccccagactcctcacttatgctgataatcagcggccctcaggggtccctgaccgattctctggctccaagtctggcacctcagcctccctggccatcagtggcctccagtctgaggatgaggctgactattattgtgcagcatgggatgacagcctgaatggtgttgtgttcggaggaggcaccaagctgaccgtccta

SEQ ID NO: 111 - protein, 8 aa, Artificial Sequence
  amino acid sequence of C2037 H CDR1
GYTFTGYY

SEQ ID NO: 112 - protein, 8 aa, Artificial Sequence
  amino acid sequence of C2037 H CDR2
INPNSGGT

SEQ ID NO: 113 - protein, 8 aa, Artificial Sequence
  amino acid sequence of C2037 H CDR3
ASGKSNYP

SEQ ID NO: 114 - protein, 8 aa, Artificial Sequence
  amino acid sequence of C2037 L CDR1
NSNIGSRT

SEQ ID NO: 115 - protein, 3 aa, Artificial Sequence
  amino acid sequence of C2037 L CDR2
GNN

SEQ ID NO: 116 - protein, 11 aa, Artificial Sequence
  amino acid sequence of C2037 L CDR3
AAWDDSLSGVV

SEQ ID NO: 117 - protein, 8 aa, Artificial Sequence
  amino acid sequence of C3048 H CDR1
GGTFSRYA

SEQ ID NO: 118 - protein, 8 aa, Artificial Sequence
  amino acid sequence of C3048 H CDR2
IIPIFGTA

SEQ ID NO: 119 - protein, 16 aa, Artificial Sequence
  amino acid sequence of C3048 H CDR3
ALEGFRDSLKRNPFDI

SEQ ID NO: 120 - protein, 8 aa, Artificial Sequence
  amino acid sequence of C3048 L CDR1
TTNIGSNY

SEQ ID NO: 121 - protein, 3 aa, Artificial Sequence
  amino acid sequence of C3048 L CDR2
ENN

SEQ ID NO: 122 - protein, 11 aa, Artificial Sequence
  amino acid sequence of C3048 L CDR3
STWDSSLNTGL

SEQ ID NO: 123 - protein, 8 aa, Artificial Sequence
  amino acid sequence of C3015 H CDR1
GFTFRNYA

SEQ ID NO: 124 - protein, 8 aa, Artificial Sequence
  amino acid sequence of C3015 H CDR2
ISGTGGST

SEQ ID NO: 125 - protein, 14 aa, Artificial Sequence
  amino acid sequence of C3015 H CDR3
AKSRSMSINYGMDV

SEQ ID NO: 126 - protein, 6 aa, Artificial Sequence
  amino acid sequence of C3015 L CDR1
NLGDRY

SEQ ID NO: 127 - protein, 3 aa, Artificial Sequence
  amino acid sequence of C3015 L CDR2
QDT

SEQ ID NO: 128 - protein, 9 aa, Artificial Sequence
  amino acid sequence of C3015 L CDR3
QAWDSNTVV

SEQ ID NO: 129 - protein, 8 aa, Artificial Sequence
  amino acid sequence of C3022 H CDR1
GYTFTGYY

SEQ ID NO: 130 - protein, 8 aa, Artificial Sequence
  amino acid sequence of C3022 H CDR2
INPNSGGT

SEQ ID NO: 131 - protein, 8 aa, Artificial Sequence
  amino acid sequence of C3022 H CDR3
ASGSVRHP

SEQ ID NO: 132 - protein, 8 aa, Artificial Sequence
  amino acid sequence of C3022 L CDR1
RSNIGGNI

SEQ ID NO: 133 - protein, 3 aa, Artificial Sequence
  amino acid sequence of C3022 L CDR2
ADN

SEQ ID NO: 134 - protein, 11 aa, Artificial Sequence
  amino acid sequence of C3022 L CDR3
AAWDDSLNGVV

SEQ ID NO: 135 - protein, 110 aa, Artificial Sequence
  amino acid sequence of C3022 VL
QPVLTQPPSASGTPGQRVTISCSGSRSNIGGNIVSWYQQFPGTAPRLLTYADNQRPSGVPDRFSGSKSGTSASLAISGLQSEDEADYYCAAWDDSLNGVVFGGGTKLTVL

SEQ ID NO: 136 - DNA, 1392 bp, Artificial Sequence
  nucleotide sequence of C2037 H in IgG form
atgaaacacctgtggttcttcctcctgctggtggcagctcccagatgggtgctgagccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagtgggaagagtaactacccctggggccagggaaccctggtcaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 137 - DNA, 708 bp, Artificial Sequence
  nucleotide sequence of C2037 L in IgG form
atggtgctgcagacccaggtgttcatctccctgctgctgtggatctccggcgcgtacggccagcctgtgctgactcagccaccctcagcgtctgggacccccgggcagagggtcaccatctcttgttctggaagcaactccaacatcggcagtagaactgtgaattggtaccagcacctcccaggaacggcccccagactcctcatctatggtaataatcagtggccctcaggggtccctgaccgattctctggctctaagtctggctcctcagcctccctggccatcagtgggctccggtccgaggatgaggctgattattactgtgcagcatgggatgacagcctgagtggtgtggtattcggcggagggaccaagttaactgtgcttggccagcctaaggctgcccctagcgtgaccctgttccctccttccagcgaggagcttcaagctaacaaggccaccctggtgtgtcttatctctgacttctaccctggcgctgtgaccgtggcctggaaggctgacagctcccctgtgaaggccggagtggagaccaccacacctagcaagcagtctaacaacaagtacgctgccagctcctacctgagccttacccctgagcagtggaagtctcacagaagctactcctgtcaagtgacccacgagggcagcaccgtggagaagaccgtggctcctaccgagtgttcc

SEQ ID NO: 138 - DNA, 1416 bp, Artificial Sequence
  nucleotide sequence of C3048 H in IgG form
atgaaacacctgtggttcttcctcctgctggtggcagctcccagatgggtgctgagccaggtgcagctggtgcagtctggggctgaggtgaagaagcctgggtcctcggtgaaggtctcctgcaaggcttctggaggcaccttcagcaggtatgctatcagctgggtgcgacaggcccctggacaagggcttgagtggatgggagggatcatccctatctttggaacagcaaactacgcacagaagttccagggcagagtcacaattaccgcggaccaatccacgagaacagcctacatggacctgggcagactgagatctgaggacacggccgtgtattattgtgctctcgagggattcagggactcattaaaacgaaatccttttgatatctggggccaagggacaatggtcaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 139 - DNA, 708 bp, Artificial Sequence
  nucleotide sequence of C3048 L in IgG form
atggtgctgcagacccaggtgttcatctccctgctgctgtggatctccggcgcgtacggccagtctgtcgtgacgcagccgccctcagtgtctgcggccccaggacagaaggtcaccatctcctgctctggaggcaccaccaacattgggagtaattatgtatcctggtatcagcagctcccaggaacagcccccaaattcctcatgtatgaaaataataagcgaccctcaggcattcctgaccgattctctggctccaagtctggcacgtcagccaccctggccatcaccggactccagactggggacgaggccacttattactgctcaacatgggatagcagcctgaatactgggctattcggcggagggaccaagttaactgtgcttggccagcctaaggctgcccctagcgtgaccctgttccctccttccagcgaggagcttcaagctaacaaggccaccctggtgtgtcttatctctgacttctaccctggcgctgtgaccgtggcctggaaggctgacagctcccctgtgaaggccggagtggagaccaccacacctagcaagcagtctaacaacaagtacgctgccagctcctacctgagccttacccctgagcagtggaagtctcacagaagctactcctgtcaagtgacccacgagggcagcaccgtggagaagaccgtggctcctaccgagtgttcc

SEQ ID NO: 140 - DNA, 1410 bp, Artificial Sequence
  nucleotide sequence of C3015 H in IgG form
atgaaacacctgtggttcttcctcctgctggtggcagctcccagatgggtgctgagcgaggtgcagctggtggagtccgggggaggcgtggtccagcctgggaggtccctgagactctcctgtgcagcctctggattcacctttaggaactatgccatgagctgggtccgccaggctccagggaaggggctggagtgggtctcaggtattagtggtactggtggtagcacatactacgcagactccgtgaagggccggttcaccatctccagagacaattccaaggacacactatatctgcaattgaacagcctgagagccgaggacacggccgtatattactgtgcgaaatcccgtagtatgagtatcaactacggtatggacgtctggggccaagggaccacggtcaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 141 - DNA, 696 bp, Artificial Sequence
  nucleotide sequence of C3015 L in IgG form
atggtgctgcagacccaggtgttcatctccctgctgctgtggatctccggcgcgtacggctcctatgagctgactcagccaccctcagtgtccgtgtccccaggacagacagtcagcatcacctgctctggagataatttgggtgatagatatgcctcctggtatcagcagaagccaggccagtcccctgtgcaggtcatctatcaagataccaagcggccctcagggatccctgagcgattctctggctccaactctgggaacacagccactctgaccatcagcgggacccaggctatggatgaggctgactattactgtcaggcgtgggacagcaacactgtggtattcggcggagggaccaagttaactgtgcttggccagcctaaggctgcccctagcgtgaccctgttccctccttccagcgaggagcttcaagctaacaaggccaccctggtgtgtcttatctctgacttctaccctggcgctgtgaccgtggcctggaaggctgacagctcccctgtgaaggccggagtggagaccaccacacctagcaagcagtctaacaacaagtacgctgccagctcctacctgagccttacccctgagcagtggaagtctcacagaagctactcctgtcaagtgacccacgagggcagcaccgtggagaagaccgtggctcctaccgagtgttcc

SEQ ID NO: 142 - DNA, 1392 bp, Artificial Sequence
  nucleotide sequence of C3022 H in IgG form
atgaaacacctgtggttcttcctcctgctggtggcagctcccagatgggtgctgagccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcgggctgagatctgacgacacggccgtttattactgtgcgagtggtagtgtaaggcatccctggggccagggaaccctggtcaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 143 - DNA, 708 bp, Artificial Sequence
  nucleotide sequence of C3022 L in IgG form
atggtgctgcagacccaggtgttcatctccctgctgctgtggatctccggcgcgtacggccagcctgtgctgactcagccaccctcagcgtctgggacccccgggcagagggtcaccatctcttgttctggaagcaggtccaacatcggaggtaatattgtaagttggtaccagcagttcccaggaacggcccccagactcctcacttatgctgataatcagcggccctcaggggtccctgaccgattctctggctccaagtctggcacctcagcctccctggccatcagtggcctccagtctgaggatgaggctgactattattgtgcagcatgggatgacagcctgaatggtgttgtgttcggaggaggcaccaagttaactgtgcttggccagcctaaggctgcccctagcgtgaccctgttccctccttccagcgaggagcttcaagctaacaaggccaccctggtgtgtcttatctctgacttctaccctggcgctgtgaccgtggcctggaaggctgacagctcccctgtgaaggccggagtggagaccaccacacctagcaagcagtctaacaacaagtacgctgccagctcctacctgagccttacccctgagcagtggaagtctcacagaagctactcctgtcaagtgacccacgagggcagcaccgtggagaagaccgtggctcctaccgagtgttcc

SEQ ID NO: 144 - protein, 464 aa, Artificial Sequence
  amino acid sequence of C2037 H in IgG form
MKHLWFFLLLVAAPRWVLSQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCASGKSNYPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 236 aa, Artificial Sequence
  amino acid sequence of C2037 L in IgG form
MVLQTQVFISLLLWISGAYGQPVLTQPPSASGTPGQRVTISCSGSNSNIGSRTVNWYQHLPGTAPRLLIYGNNQWPSGVPDRFSGSKSGSSASLAISGLRSEDEADYYCAAWDDSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 146 - protein, 472 aa, Artificial Sequence
  amino acid sequence of C3048 H in IgG form
MKHLWFFLLLVAAPRWVLSQVQLVQSGAEVKKPGSSVKVSCKASGGTFSRYAISWVRQAPGQGLEWMGGIIPIFGTANYAQKFQGRVTITADQSTRTAYMDLGRLRSEDTAVYYCALEGFRDSLKRNPFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 236 aa, Artificial Sequence
  amino acid sequence of C3048 L in IgG form
MVLQTQVFISLLLWISGAYGQSVVTQPPSVSAAPGQKVTISCSGGTTNIGSNYVSWYQQLPGTAPKFLMYENNKRPSGIPDRFSGSKSGTSATLAITGLQTGDEATYYCSTWDSSLNTGLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 148 - protein, 470 aa, Artificial Sequence
  amino acid sequence of C3015 H in IgG form
MKHLWFFLLLVAAPRWVLSEVQLVESGGGVVQPGRSLRLSCAASGFTFRNYAMSWVRQAPGKGLEWVSGISGTGGSTYYADSVKGRFTISRDNSKDTLYLQLNSLRAEDTAVYYCAKSRSMSINYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 232 aa, Artificial Sequence
  amino acid sequence of C3015 L in IgG form
MVLQTQVFISLLLWISGAYGSYELTQPPSVSVSPGQTVSITCSGDNLGDRYASWYQQKPGQSPVQVIYQDTKRPSGIPERFSGSNSGNTATLTISGTQAMDEADYYCQAWDSNTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 150 - protein, 464 aa, Artificial Sequence
  amino acid sequence of C3022 H in IgG form
MKHLWFFLLLVAAPRWVLSQVQLVQSGAEVKKPGASVKVSCKASGYTFTGYYMHWVRQAPGQGLEWMGRINPNSGGTNYAQKFQGRVTMTRDTSISTAYMELSGLRSDDTAVYYCASGSVRHPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 236 aa, Artificial Sequence
  amino acid sequence of C3022 L in IgG form
MVLQTQVFISLLLWISGAYGQPVLTQPPSASGTPGQRVTISCSGSRSNIGGNIVSWYQQFPGTAPRLLTYADNQRPSGVPDRFSGSKSGTSASLAISGLQSEDEADYYCAAWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 152 - DNA, 354 bp, Rattus norvegicus
gaggtgcagttggtggagtctgggggaggcctggtgcagcctggaagggccctgaaactctcctgtgtagtctctggagtcacattcaattactacgggatgagctggatccgccaggctccagggaaggggctggagtgggttgcatccattactaattctggtggtagaatttactatccagactctgtgaagggccgattcactatctccagagaaaatacacaaaagaccctatacctacaaatgaacagtctgaggtctgaggacacggccacttattactgtactctcgatggtcgcgatggttgggttgcttactggggccaaggcactctggtcactgtctcttca

SEQ ID NO: 153 - DNA, 327 bp, Rattus norvegicus
cagtttgtgcttactcagccaaactctgtgtctacgaatctcggaaccacagtcgaactgtcttgcaagcgcaacactgggaacattggaagcaattatgtgaactggtaccagcagcatgagggaagatctcccaccactattatttatagggatgataagagaccagatggagtttctgacaggttctctgggtccattgacagatcttccaagtcagccctcctgacaatcaataatgtgcagactgaagatgaagctgactacttctgtcagtcttacagtagtggttttattttcggcggtggaaccaagctcactgtccta

SEQ ID NO: 154 - DNA, 867 bp, Artificial Sequence
  nucleotide sequence of C3E-7000
atgaaacacctgtggttcttcctcctgctggtggcagctcccagatgggtgctgagcgaggtgcagttggtggagtctgggggaggcctggtgcagcctggaagggccctgaaactctcctgtgtagtctctggagtcacattcaattactacgggatgagctggatccgccaggctccagggaaggggctggagtgggttgcatccattactaattctggtggtagaatttactatccagactctgtgaagggccgattcactatctccagagaaaatacacaaaagaccctatacctacaaatgaacagtctgaggtctgaggacacggccacttattactgtactctcgatggtcgcgatggttgggttgcttactggggccaaggcactctggtcactgtctcttcaggtggaggcggttcaggcggaggtggcagcggcggtggcgggagtcagtttgtgcttactcagccaaactctgtgtctacgaatctcggaaccacagtcgaactgtcttgcaagcgcaacactgggaacattggaagcaattatgtgaactggtaccagcagcatgagggaagatctcccaccactattatttatagggatgataagagaccagatggagtttctgacaggttctctgggtccattgacagatcttccaagtcagccctcctgacaatcaataatgtgcagactgaagatgaagctgactacttctgtcagtcttacagtagtggttttattttcggcggtggaaccaagctcactgtcctaggcgcgtctgcggccgcaggatccggtggtgattacaaagatgatgacgataaaggtgcagcggcgcatcaccatcatcaccac

SEQ ID NO: 155 - protein, 118 aa, Artificial Sequence
  amino acid sequence of C3E-7034_VH
EVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSS

SEQ ID NO: 156 - protein, 109 aa, Artificial Sequence
  amino acid sequence of C3E-7034_VL
NFMLTQPHSVSESPGKTVTISCKRNTGNIGSNYVNWYQQHEGSSPTTIIYRDDKRPDGVSDRFSGSIDRSSKSASLTISNLKTEDEADYFCQSYSSGFIFGGGTKLTVL

SEQ ID NO: 157 - DNA, 864 bp, Artificial Sequence
  nucleotide sequence of C3E-7034
atgaaacacctgtggttcttcctcctgctggtggcagctcccagatgggtgctgagcggagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctgacccagccccacagtgtgtcagagagccctggcaagactgtcaccatctcttgtaaaaggaacaccggaaatattggcagtaactacgtgaattggtatcagcagcatgaagggtctagtccaaccacaatcatctaccgggacgataagagacccgacggggtgtccgatcgattctccggatctatcgaccggtcaagcaagagtgcttcactgaccattagcaatctgaaaacagaggacgaagcagattacttttgccagtcctattcctctggcttcatctttggaggcgggactaaactgaccgtgctgggcgcggccgcaggtgcaggtggtgattacaaagatgatgacgataaaggtgcagcggcgcatcaccatcatcaccac

SEQ ID NO: 158 - protein, 107 aa, Artificial Sequence
  amino acid sequence of C3E-7035_VL
QAVLTQPSSVSGVPGQRVTISCKRNTGNIGSNYVNWYQQLPGTAPKLLIYRDDKRPSGVPDRFSGSKSGTSASLAITGFQAEDEADYYCQSYSSGFIFGGGTKLTVL

SEQ ID NO: 159 - DNA, 864 bp, Artificial Sequence
  nucleotide sequence of C3E-7035
atgaaacacctgtggttcttcctcctgctggtggcagctcccagatgggtgctgagcggagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaatggcccaggctgtgctcactcagccgtcctctgtttctggcgtacctggccaacgggtgaccattagctgtaaaaggaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgctgggcgcggccgcaggtgcaggtggtgattacaaagatgatgacgataaaggtgcagcggcgcatcaccatcatcaccac

SEQ ID NO: 160 - protein, 107 aa, Artificial Sequence
  amino acid sequence of C3E-7036_VL
NFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVL

SEQ ID NO: 161 - DNA, 858 bp, Artificial Sequence
  nucleotide sequence of C3E-7036
atgaaacacctgtggttcttcctcctgctggtggcagctcccagatgggtgctgagcggagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctcactcagccgtcctctgtttctggcgtacctggccaacgggtgaccattagctgtacgggtaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgttgggtgcggccgcaggtgcaggtggtgattacaaagatgatgacgataaaggtgcagcggcgcatcaccatcatcaccac

SEQ ID NO: 162 - DNA, 1599 bp, Artificial Sequence
  nucleotide_sequence_of_C2037-C3E-7034
atgaaacacctgtggttcttcctcctgctggtggcagctcccagatgggtgctgagcggacaggtgcagctggtgcagtctggcgccgaagtgaagaaaccaggcgccagcgtgaaggtgtcctgcaaggccagcggctacacctttaccggctactacatgcactgggtgcgccaggctccaggccagggactggaatggatgggccggatcaaccccaatagcggcggcaccaactacgcccagaaattccagggcagagtgaccatgacccgggacaccagcatcagcaccgcctacatggaactgagccggctgagaagcgacgacaccgccgtgtactactgcgccagcggcaagagcaactacccttggggccagggcacactcgtgaccgtgtctagcggaggcggaggatctggcggcggaggaagtggcggagggggatctcagcctgtgctgacacagcctcctagcgcctctggcacacctggccagagagtgacaatcagctgcagcggcagcaacagcaacatcggcagccggaccgtgaactggtatcagcatctgcctggcaccgcccccagactgctgatctacggcaacaaccagtggcccagcggcgtgcccgatagattcagcggctctaagagcggcagctccgccagcctggccatctctggactgagatccgaggacgaggccgactactactgtgccgcctgggacgatagcctgagcggcgtggtgtttggcggaggcaccaagctgacagtgctgggcggaggcggttcagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctgacccagccccacagtgtgtcagagagccctggcaagactgtcaccatctcttgtaaaaggaacaccggaaatattggcagtaactacgtgaattggtatcagcagcatgaagggtctagtccaaccacaatcatctaccgggacgataagagacccgacggggtgtccgatcgattctccggatctatcgaccggtcaagcaagagtgcttcactgaccattagcaatctgaaaacagaggacgaagcagattacttttgccagtcctattcctctggcttcatctttggaggcgggactaaactgaccgtgctgggcgcggccgcaggtgcaggtggtgattacaaagatgatgacgataaaggtgcagcggcgcatcaccatcatcaccac

SEQ ID NO: 163 - DNA, 1623 bp, Artificial Sequence
  nucleotide sequence of C3048-C3E-7034
atgaaacacctgtggttcttcctcctgctggtggcagctcccagatgggtgctgagcggacaggtgcagctggtgcagtctggcgccgaagtgaagaaacccggcagcagcgtgaaggtgtcctgcaaggctagcggcggcaccttcagcagatacgccatctcttgggtgcgccaggcccctggacagggcctggaatggatgggcggcatcatccccatcttcggcaccgccaactacgcccagaaattccagggcagagtgaccatcaccgccgaccagagcacccggaccgcctatatggatctgggccggctgagaagcgaggacaccgccgtgtactactgcgccctggaaggcttccgggacagcctgaagagaaaccccttcgacatctggggccagggcacaatggtcaccgtgtctagcggaggcggaggatctggcggcggaggaagtggcggagggggatctcagtctgtcgtgacccagcctcctagcgtgtcagccgccccaggccagaaagtgacaatcagctgttctggcggcaccaccaacatcggcagcaactacgtgtcctggtatcagcagctgcccggaaccgcccccaagttcctgatgtacgagaacaacaagcggcccagcggcatccccgacagattcagcggcagcaagagcggcacaagcgccacactggccatcaccggactgcagacaggcgacgaggccacctactactgctccacctgggacagcagcctgaacaccggcctgtttggcggaggcaccaagctgacagtgctgggcggaggcggttcagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctgacccagccccacagtgtgtcagagagccctggcaagactgtcaccatctcttgtaaaaggaacaccggaaatattggcagtaactacgtgaattggtatcagcagcatgaagggtctagtccaaccacaatcatctaccgggacgataagagacccgacggggtgtccgatcgattctccggatctatcgaccggtcaagcaagagtgcttcactgaccattagcaatctgaaaacagaggacgaagcagattacttttgccagtcctattcctctggcttcatctttggaggcgggactaaactgaccgtgctgggcgcggccgcaggtgcaggtggtgattacaaagatgatgacgataaaggtgcagcggcgcatcaccatcatcaccac

SEQ ID NO: 164 - DNA, 1599 bp, Artificial Sequence
  nucleotide sequence of C3022-C3E-7034
atgaaacacctgtggttcttcctcctgctggtggcagctcccagatgggtgctgagcggacaggtgcagctggtgcagtctggcgccgaagtgaagaaaccaggcgccagcgtgaaggtgtcctgcaaggccagcggctacacctttaccggctactacatgcactgggtgcgccaggctccaggccagggactggaatggatgggccggatcaaccccaatagcggcggcaccaactacgcccagaaattccagggcagagtgaccatgacccgggacaccagcatcagcaccgcctacatggaactgagcggcctgagaagcgacgacaccgccgtgtactactgtgcctctggctctgtgcggcacccttggggacagggaacactcgtgaccgtgtccagcggtggaggcggttcaggcggaggtggcagcggcggtggcgggagtcagcctgtgctgacacagcctccaagcgcctctggcacacctggccagagagtgacaatcagctgcagcggcagcagaagcaacatcggcggcaacatcgtgtcctggtatcagcagttccccggcaccgcccctagactgctgacctacgccgacaaccagaggcctagcggcgtgcccgatagattcagcggctctaagagcggcaccagcgccagcctggccatctctggcctgcagtctgaggacgaggccgactactattgcgccgcctgggacgacagcctgaacggcgtggtgtttggcggaggcaccaagctgacagtgctgggcggaggcggttcagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctgacccagccccacagtgtgtcagagagccctggcaagactgtcaccatctcttgtaaaaggaacaccggaaatattggcagtaactacgtgaattggtatcagcagcatgaagggtctagtccaaccacaatcatctaccgggacgataagagacccgacggggtgtccgatcgattctccggatctatcgaccggtcaagcaagagtgcttcactgaccattagcaatctgaaaacagaggacgaagcagattacttttgccagtcctattcctctggcttcatctttggaggcgggactaaactgaccgtgctgggcgcggccgcaggtgcaggtggtgattacaaagatgatgacgataaaggtgcagcggcgcatcaccatcatcaccac

SEQ ID NO: 165 - DNA, 1599 bp, Artificial Sequence
  nucleotide sequence of C2037-C3E-7035
atgaaacacctgtggttcttcctcctgctggtggcagctcccagatgggtgctgagcggacaggtgcagctggtgcagtctggcgccgaagtgaagaaaccaggcgccagcgtgaaggtgtcctgcaaggccagcggctacacctttaccggctactacatgcactgggtgcgccaggctccaggccagggactggaatggatgggccggatcaaccccaatagcggcggcaccaactacgcccagaaattccagggcagagtgaccatgacccgggacaccagcatcagcaccgcctacatggaactgagccggctgagaagcgacgacaccgccgtgtactactgcgccagcggcaagagcaactacccttggggccagggcacactcgtgaccgtgtctagcggaggcggaggatctggcggcggaggaagtggcggagggggatctcagcctgtgctgacacagcctcctagcgcctctggcacacctggccagagagtgacaatcagctgcagcggcagcaacagcaacatcggcagccggaccgtgaactggtatcagcatctgcctggcaccgcccccagactgctgatctacggcaacaaccagtggcccagcggcgtgcccgatagattcagcggctctaagagcggcagctccgccagcctggccatctctggactgagatccgaggacgaggccgactactactgtgccgcctgggacgatagcctgagcggcgtggtgtttggcggaggcaccaagctgacagtgctgggcggaggcggttcagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaatggcccaggctgtgctcactcagccgtcctctgtttctggcgtacctggccaacgggtgaccattagctgtaaaaggaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgttgggcgcggccgcaggtgcaggtggtgattacaaagatgatgacgataaaggtgcagcggcgcatcaccatcatcaccac

SEQ ID NO: 166 - DNA, 1623 bp, Artificial Sequence
  nucleotide sequence of C3048-C3E-7035
atgaaacacctgtggttcttcctcctgctggtggcagctcccagatgggtgctgagcggacaggtgcagctggtgcagtctggcgccgaagtgaagaaacccggcagcagcgtgaaggtgtcctgcaaggctagcggcggcaccttcagcagatacgccatctcttgggtgcgccaggcccctggacagggcctggaatggatgggcggcatcatccccatcttcggcaccgccaactacgcccagaaattccagggcagagtgaccatcaccgccgaccagagcacccggaccgcctatatggatctgggccggctgagaagcgaggacaccgccgtgtactactgcgccctggaaggcttccgggacagcctgaagagaaaccccttcgacatctggggccagggcacaatggtcaccgtgtctagcggaggcggaggatctggcggcggaggaagtggcggagggggatctcagtctgtcgtgacccagcctcctagcgtgtcagccgccccaggccagaaagtgacaatcagctgttctggcggcaccaccaacatcggcagcaactacgtgtcctggtatcagcagctgcccggaaccgcccccaagttcctgatgtacgagaacaacaagcggcccagcggcatccccgacagattcagcggcagcaagagcggcacaagcgccacactggccatcaccggactgcagacaggcgacgaggccacctactactgctccacctgggacagcagcctgaacaccggcctgtttggcggaggcaccaagctgacagtgctgggcggaggcggttcagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaatggcccaggctgtgctcactcagccgtcctctgtttctggcgtacctggccaacgggtgaccattagctgtaaaaggaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgttgggcgcggccgcaggtgcaggtggtgattacaaagatgatgacgataaaggtgcagcggcgcatcaccatcatcaccac

SEQ ID NO: 167 - DNA, 1599 bp, Artificial Sequence
  nucleotide sequence of C3022-C3E-7035
atgaaacacctgtggttcttcctcctgctggtggcagctcccagatgggtgctgagcggacaggtgcagctggtgcagtctggcgccgaagtgaagaaaccaggcgccagcgtgaaggtgtcctgcaaggccagcggctacacctttaccggctactacatgcactgggtgcgccaggctccaggccagggactggaatggatgggccggatcaaccccaatagcggcggcaccaactacgcccagaaattccagggcagagtgaccatgacccgggacaccagcatcagcaccgcctacatggaactgagcggcctgagaagcgacgacaccgccgtgtactactgtgcctctggctctgtgcggcacccttggggacagggaacactcgtgaccgtgtccagcggtggaggcggttcaggcggaggtggcagcggcggtggcgggagtcagcctgtgctgacacagcctccaagcgcctctggcacacctggccagagagtgacaatcagctgcagcggcagcagaagcaacatcggcggcaacatcgtgtcctggtatcagcagttccccggcaccgcccctagactgctgacctacgccgacaaccagaggcctagcggcgtgcccgatagattcagcggctctaagagcggcaccagcgccagcctggccatctctggcctgcagtctgaggacgaggccgactactattgcgccgcctgggacgacagcctgaacggcgtggtgtttggcggaggcaccaagctgacagtgctgggcggaggcggttcagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaatggcccaggctgtgctcactcagccgtcctctgtttctggcgtacctggccaacgggtgaccattagctgtaaaaggaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgttgggcgcggccgcaggtgcaggtggtgattacaaagatgatgacgataaaggtgcagcggcgcatcaccatcatcaccac

SEQ ID NO: 168 - DNA, 1593 bp, Artificial Sequence
  nucleotide sequence of C2037-C3E-7036
atgaaacacctgtggttcttcctcctgctggtggcagctcccagatgggtgctgagcggacaggtgcagctggtgcagtctggcgccgaagtgaagaaaccaggcgccagcgtgaaggtgtcctgcaaggccagcggctacacctttaccggctactacatgcactgggtgcgccaggctccaggccagggactggaatggatgggccggatcaaccccaatagcggcggcaccaactacgcccagaaattccagggcagagtgaccatgacccgggacaccagcatcagcaccgcctacatggaactgagccggctgagaagcgacgacaccgccgtgtactactgcgccagcggcaagagcaactacccttggggccagggcacactcgtgaccgtgtctagcggaggcggaggatctggcggcggaggaagtggcggagggggatctcagcctgtgctgacacagcctcctagcgcctctggcacacctggccagagagtgacaatcagctgcagcggcagcaacagcaacatcggcagccggaccgtgaactggtatcagcatctgcctggcaccgcccccagactgctgatctacggcaacaaccagtggcccagcggcgtgcccgatagattcagcggctctaagagcggcagctccgccagcctggccatctctggactgagatccgaggacgaggccgactactactgtgccgcctgggacgatagcctgagcggcgtggtgtttggcggaggcaccaagctgacagtgctgggcggaggcggttcagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctcactcagccgtcctctgtttctggcgtacctggccaacgggtgaccattagctgtacgggtaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgttgggcgcggccgcaggtgcaggtggtgattacaaagatgatgacgataaaggtgcagcggcgcatcaccatcatcaccac

SEQ ID NO: 169 - DNA, 1617 bp, Artificial Sequence
  nucleotide sequence of C3048-C3E-7036
atgaaacacctgtggttcttcctcctgctggtggcagctcccagatgggtgctgagcggacaggtgcagctggtgcagtctggcgccgaagtgaagaaacccggcagcagcgtgaaggtgtcctgcaaggctagcggcggcaccttcagcagatacgccatctcttgggtgcgccaggcccctggacagggcctggaatggatgggcggcatcatccccatcttcggcaccgccaactacgcccagaaattccagggcagagtgaccatcaccgccgaccagagcacccggaccgcctatatggatctgggccggctgagaagcgaggacaccgccgtgtactactgcgccctggaaggcttccgggacagcctgaagagaaaccccttcgacatctggggccagggcacaatggtcaccgtgtctagcggaggcggaggatctggcggcggaggaagtggcggagggggatctcagtctgtcgtgacccagcctcctagcgtgtcagccgccccaggccagaaagtgacaatcagctgttctggcggcaccaccaacatcggcagcaactacgtgtcctggtatcagcagctgcccggaaccgcccccaagttcctgatgtacgagaacaacaagcggcccagcggcatccccgacagattcagcggcagcaagagcggcacaagcgccacactggccatcaccggactgcagacaggcgacgaggccacctactactgctccacctgggacagcagcctgaacaccggcctgtttggcggaggcaccaagctgacagtgctgggcggaggcggttcagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctcactcagccgtcctctgtttctggcgtacctggccaacgggtgaccattagctgtacgggtaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgttgggcgcggccgcaggtgcaggtggtgattacaaagatgatgacgataaaggtgcagcggcgcatcaccatcatcaccac

SEQ ID NO: 170 - DNA, 1593 bp, Artificial Sequence
  nucleotide sequence of C3022-C3E-7036
atgaaacacctgtggttcttcctcctgctggtggcagctcccagatgggtgctgagcggacaggtgcagctggtgcagtctggcgccgaagtgaagaaaccaggcgccagcgtgaaggtgtcctgcaaggccagcggctacacctttaccggctactacatgcactgggtgcgccaggctccaggccagggactggaatggatgggccggatcaaccccaatagcggcggcaccaactacgcccagaaattccagggcagagtgaccatgacccgggacaccagcatcagcaccgcctacatggaactgagcggcctgagaagcgacgacaccgccgtgtactactgtgcctctggctctgtgcggcacccttggggacagggaacactcgtgaccgtgtccagcggtggaggcggttcaggcggaggtggcagcggcggtggcgggagtcagcctgtgctgacacagcctccaagcgcctctggcacacctggccagagagtgacaatcagctgcagcggcagcagaagcaacatcggcggcaacatcgtgtcctggtatcagcagttccccggcaccgcccctagactgctgacctacgccgacaaccagaggcctagcggcgtgcccgatagattcagcggctctaagagcggcaccagcgccagcctggccatctctggcctgcagtctgaggacgaggccgactactattgcgccgcctgggacgacagcctgaacggcgtggtgtttggcggaggcaccaagctgacagtgctgggcggaggcggttcagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaattccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctcactcagccgtcctctgtttctggcgtacctggccaacgggtgaccattagctgtacgggtaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgttgggcgcggccgcaggtgcaggtggtgattacaaagatgatgacgataaaggtgcagcggcgcatcaccatcatcaccac

SEQ ID NO: 171 - protein, 533 aa, Artificial Sequence
  amino acid sequence of C2037-C3E-7034
MKHLWFFLLLVAAPRWVLSGQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCASGKSNYPWGQGTLVTVSSGGGGSGGGGSGGGGSQPVLTQPPSASGTPGQRVTISCSGSNSNIGSRTVNWYQHLPGTAPRLLIYGNNQWPSGVPDRFSGSKSGSSASLAISGLRSEDEADYYCAAWDDSLSGVVFGGGTKLTVLGGGGSEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRDDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 172 - protein, 541 aa, Artificial Sequence
  amino acid sequence of C3048-C3E-7034
MKHLWFFLLLVAAPRWVLSGQVQLVQSGAEVKKPGSSVKVSCKASGGTFSRYAISWVRQAPGQGLEWMGGIIPIFGTANYAQKFQGRVTITADQSTRTAYMDLGRLRSEDTAVYYCALEGFRDSLKRNPFDIWGQGTMVTVSSGGGGSGGGGSGGGGSQSVVTQPPSVSAAPGQKVTISCSGGTTNIGSNYVSWYQQLPGTAPKFLMYENNKRPSGIPDRFSGSKSGTSATLAITGLQTGDEATYYCSTWDSSLNTGLFGGGTKLTVLGGGGSEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRDDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 173 - protein, 533 aa, Artificial Sequence
  amino acid sequence of C3022-C3E-7034
MKHLWFFLLLVAAPRWVLSGQVQLVQSGAEVKKPGASVKVSCKASGYTFTGYYMHWVRQAPGQGLEWMGRINPNSGGTNYAQKFQGRVTMTRDTSISTAYMELSGLRSDDTAVYYCASGSVRHPWGQGTLVTVSSGGGGSGGGGSGGGGSQPVLTQPPSASGTPGQRVTISCSGSRSNIGGNIVSWYQQFPGTAPRLLTYADNQRPSGVPDRFSGSKSGTSASLAISGLQSEDEADYYCAAWDDSLNGVVFGGGTKLTVLGGGGSEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRDDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 174 - protein, 533 aa, Artificial Sequence
  amino acid sequence of C2037-C3E-7035
MKHLWFFLLLVAAPRWVLSGQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCASGKSNYPWGQGTLVTVSSGGGGSGGGGSGGGGSQPVLTQPPSASGTPGQRVTISCSGSNSNIGSRTVNWYQHLPGTAPRLLIYGNNQWPSGVPDRFSGSKSGSSASLAISGLRSEDEADYYCAAWDDSLSGVVFGGGTKLTVLGGGGSEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSMAQAVLTQPSSVSGVPGQRVTISCKRNTGNIGSNYVNWYQQLPGTAPKLLIYRDDKRPSGVPDRFSGSKSGTSASLAITGFQAEDEADYYCQSYSSGFIFGGGTKLTVLGAAAGAGGDYKDDDDKGAAAHHHHHH

SEQ ID NO: 175 - protein, 541 aa, Artificial Sequence
  amino acid sequence of C3048-C3E-7035
MKHLWFFLLLVAAPRWVLSGQVQLVQSGAEVKKPGSSVKVSCKASGGTFSRYAISWVRQAPGQGLEWMGGIIPIFGTANYAQKFQGRVTITADQSTRTAYMDLGRLRSEDTAVYYCALEGFRDSLKRNPFDIWGQGTMVTVSSGGGGSGGGGSGGGGSQSVVTQPPSVSAAPGQKVTISCSGGTTNIGSNYVSWYQQLPGTAPKFLMYENNKRPSGIPDRFSGSKSGTSATLAITGLQTGDEATYYCSTWDSSLNTGLFGGGTKLTVLGGGGSEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSMAQAVLTQPSSVSGVPGQRVTISCKRNTGNIGSNYVNWYQQLPGTAPKLLIYRDDKRPSGVPDRFSGSKSGTSASLAITGFQAEDEADYYCQSYSSGFIFGGGTKLTVLGAAAGAGGDYKDDDDKGAAAHHHHHH

SEQ ID NO: 176 - protein, 533 aa, Artificial Sequence
  amino acid sequence of C3022-C3E-7035
MKHLWFFLLLVAAPRWVLSGQVQLVQSGAEVKKPGASVKVSCKASGYTFTGYYMHWVRQAPGQGLEWMGRINPNSGGTNYAQKFQGRVTMTRDTSISTAYMELSGLRSDDTAVYYCASGSVRHPWGQGTLVTVSSGGGGSGGGGSGGGGSQPVLTQPPSASGTPGQRVTISCSGSRSNIGGNIVSWYQQFPGTAPRLLTYADNQRPSGVPDRFSGSKSGTSASLAISGLQSEDEADYYCAAWDDSLNGVVFGGGTKLTVLGGGGSEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSMAQAVLTQPSSVSGVPGQRVTISCKRNTGNIGSNYVNWYQQLPGTAPKLLIYRDDKRPSGVPDRFSGSKSGTSASLAITGFQAEDEADYYCQSYSSGFIFGGGTKLTVLGAAAGAGGDYKDDDDKGAAAHHHHHH

SEQ ID NO: 177 - protein, 531 aa, Artificial Sequence
  amino acid sequence of C2037-C3E-7036
MKHLWFFLLLVAAPRWVLSGQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCASGKSNYPWGQGTLVTVSSGGGGSGGGGSGGGGSQPVLTQPPSASGTPGQRVTISCSGSNSNIGSRTVNWYQHLPGTAPRLLIYGNNQWPSGVPDRFSGSKSGSSASLAISGLRSEDEADYYCAAWDDSLSGVVFGGGTKLTVLGGGGSEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVLGAAAGAGGDYKDDDDKGAAAHHHHHH

SEQ ID NO: 178 - protein, 539 aa, Artificial Sequence
  amino acid sequence of C3048-C3E-7036
MKHLWFFLLLVAAPRWVLSGQVQLVQSGAEVKKPGSSVKVSCKASGGTFSRYAISWVRQAPGQGLEWMGGIIPIFGTANYAQKFQGRVTITADQSTRTAYMDLGRLRSEDTAVYYCALEGFRDSLKRNPFDIWGQGTMVTVSSGGGGSGGGGSGGGGSQSVVTQPPSVSAAPGQKVTISCSGGTTNIGSNYVSWYQQLPGTAPKFLMYENNKRPSGIPDRFSGSKSGTSATLAITGLQTGDEATYYCSTWDSSLNTGLFGGGTKLTVLGGGGSEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVLGAAAGAGGDYKDDDDKGAAAHHHHHH

SEQ ID NO: 179 - protein, 531 aa, Artificial Sequence
  amino acid sequence of C3022-C3E-7036
MKHLWFFLLLVAAPRWVLSGQVQLVQSGAEVKKPGASVKVSCKASGYTFTGYYMHWVRQAPGQGLEWMGRINPNSGGTNYAQKFQGRVTMTRDTSISTAYMELSGLRSDDTAVYYCASGSVRHPWGQGTLVTVSSGGGGSGGGGSGGGGSQPVLTQPPSASGTPGQRVTISCSGSRSNIGGNIVSWYQQFPGTAPRLLTYADNQRPSGVPDRFSGSKSGTSASLAISGLQSEDEADYYCAAWDDSLNGVVFGGGTKLTVLGGGGSEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVLGAAAGAGGDYKDDDDKGAAAHHHHHH

SEQ ID NO: 180 - protein, 269 aa, Artificial Sequence
  amino acid sequence of C3E-7034
GEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRDDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 181 - protein, 269 aa, Artificial Sequence
  amino acid sequence of C3E-7035
GEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSMAQAVLTQPSSVSGVPGQRVTISCKRNTGNIGSNYVNWYQQLPGTAPKLLIYRDDKRPSGVPDRFSGSKSGTSASLAITGFQAEDEADYYCQSYSSGFIFGGGTKLTVLGAAAGAGGDYKDDDDKGAAAHHHHHH

SEQ ID NO: 182 - protein, 267 aa, Artificial Sequence
  amino acid sequence of C3E-7036
GEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVLGAAAGAGGDYKDDDDKGAAAHHHHHH

SEQ ID NO: 183 - protein, 8 aa, Artificial Sequence
  amino acid sequence of C3E-7000_H_CDR1
GVTFNYYG

SEQ ID NO: 184 - protein, 8 aa, Artificial Sequence
  amino acid sequence of C3E-7000_H_CDR2
ITNSGGRI

SEQ ID NO: 185 - protein, 11 aa, Artificial Sequence
  amino acid sequence of C3E-7000_H_CDR3
TLDGRDGWVAY

SEQ ID NO: 186 - protein, 8 aa, Artificial Sequence
  amino acid sequence of C3E-7000_L_CDR1
TGNIGSNY

SEQ ID NO: 187 - protein, 3 aa, Artificial Sequence
  amino acid sequence of C3E-7000_L_CDR2
RDD

SEQ ID NO: 188 - protein, 8 aa, Artificial Sequence
  amino acid sequence of C3E-7000_L_CDR3
QSYSSGFI

SEQ ID NO: 189 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 190 - DNA, 345 bp, Artificial Sequence
  nucleotide sequence of E1018 VH
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcgggctgagatctgacgacacggccgtttattactgtaagagtggtagtgtaaggcctccctggggccagggaaccctggtcaccgtctcctca

SEQ ID NO: 191 - protein, 115 aa, Artificial Sequence
  amino acid sequence of E1018 VH
QVQLVQSGAEVKKPGASVKVSCKASGYTFTGYYMHWVRQAPGQGLEWMGRINPNSGGTNYAQKFQGRVTMTRDTSISTAYMELSGLRSDDTAVYYCKSGSVRPPWGQGTLVTVSS

SEQ ID NO: 192 - DNA, 330 bp, Artificial Sequence
  nucleotide sequence of E1018 VL
cagcctgtgctgactcagccaccctcagcgtctgggacccccgggcagagggtcaccatctcttgttctggaagcaggtccaacatcggaggtaatattgtaagttggtaccagcagttcccaggaacggcccccagactcctcacttatcggaatactcggcggccctcaggggtccctgaccgattctctggctccaagtctggcacctcagcctccctggccatcagtggcctccagtctgaggatgaggctgactattattgtgcagcatgggatgacagcctggggggtgttgtgttcggaggaggcaccaagctgaccgtccta

SEQ ID NO: 193 - protein, 110 aa, Artificial Sequence
  amino acid sequence of E1018 VL
QPVLTQPPSASGTPGQRVTISCSGSRSNIGGNIVSWYQQFPGTAPRLLTYRNTRRPSGVPDRFSGSKSGTSASLAISGLQSEDEADYYCAAWDDSLGGVVFGGGTKLTVL

SEQ ID NO: 194 - DNA, 369 bp, Artificial Sequence
  nucleotide sequence of D1012 VH
caggttcagctggttcagtctggcgccgaagtgaagaaacctggcagcagcgtgaaggtgtcctgcaaagcttctggcggcaccctgagcagatacgccatctcttgggttcgacaggcccctggacaaggcctggaatggatgggaggcatcatccccatcttcggcaccgccaattacgcccagaaattccagggcagagtgaccatcaccgccgaccagtctaccagaaccgcctacatggagctgggcagactgagaagcgaggacaccgccgtgtactactgtgctctggaaggcttccgggacagcctgaagagaaaccccgtggacatctggggcagaggcaccatggttacagtgtctagc

SEQ ID NO: 195 - protein, 123 aa, Artificial Sequence
  amino acid sequence of D1012 VH
QVQLVQSGAEVKKPGSSVKVSCKASGGTLSRYAISWVRQAPGQGLEWMGGIIPIFGTANYAQKFQGRVTITADQSTRTAYMELGRLRSEDTAVYYCALEGFRDSLKRNPVDIWGRGTMVTVSS

SEQ ID NO: 196 - DNA, 330 bp, Artificial Sequence
  nucleotide sequence of D1012 VL
caatctgtggtcacacagcctccaagcgtgtcagctgccccaggccagaaagtgacaatcccttgtagcggcggcaccaccaacatcggcagcaattacgtgtcctggtatcagcagctgcccggaacagcccctaagttcctgatgtacgagaacaacaagcggcccagcggcatccccgatagattttctggcagcaagagcggcaccagcgccacactggctattacaggactggagacagaggacgaggccacctactactgtagcacctgggacagcagcctgaacgccggactttttggaggcggcacagagctgacagttctt

SEQ ID NO: 197 - protein, 110 aa, Artificial Sequence
  amino acid sequence of D1012 VL
QSVVTQPPSVSAAPGQKVTIPCSGGTTNIGSNYVSWYQQLPGTAPKFLMYENNKRPSGIPDRFSGSKSGTSATLAITGLETEDEATYYCSTWDSSLNAGLFGGGTELTVL

SEQ ID NO: 198 - DNA, 1425 bp, Artificial Sequence
  nucleotide sequence of h2B1 Fab HC_1
atgaggcctacctgggcctggtggctgttcctggtgctgctgctggccctgtgggcccccgcccggggccaggtgcagctggtggagtccggcggcggcctggtgaagcccagccagaccctgtccctgacctgcacattcagcggcttttccctgaacacatacgatatgggagtgggatggatcagacagccacctggcaagggcctggagtggatcggcaacatctggtgggacgatgacaagtactataatccctctctgaagagcagagtgaccatctctaaggatacaagcaacaatcaggcctccctgaagctgagctccgtgaccgccgccgacacagccgtgtactattgcgccaggatcgagacagtgcgggtgagccggaagggattcgcacactggggacagggcaccctggtgacagtgtctagcgctagcacaaagggacctagcgtgttcccactggccccttcctctaagtccacctctggaggaacagccgccctgggctgtagggtgaaggactatttccctgagccagtgaccgtgtcctggaactctggggccctgaccagcggagtgcacacatttcctgccgtgctgcagagctccggcctgtacagcctgtctagcgtggtgaccgtgccatcctctagcctgggcacccagacatatatctgcaacgtgaatcacaagccaagcaatacaaaggtcgacaagcgggtggagcccaagtcctgtgacaagacccacacatgcccaccctgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 199 - protein, 475 aa, Artificial Sequence
  amino acid sequence of H2B1 Fab HC_1
MRPTWAWWLFLVLLLALWAPARGQVQLVESGGGLVKPSQTLSLTCTFSGFSLNTYDMGVGWIRQPPGKGLEWIGNIWWDDDKYYNPSLKSRVTISKDTSNNQASLKLSSVTAADTAVYYCARIETVRVSRKGFAHWGQGTLVTVSSASTKGPSVFPLAPSSKSTSGGTAALGCRVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 200 - DNA, 1425 bp, Artificial Sequence
  nucleotide_sequence_of_h2B1_Fab_HC_2
atgaggcctacctgggcctggtggctgttcctggtgctgctgctggccctgtgggcccccgcccggggccaggtgcagctggtggagtccggcggcggcctggtgaagcccagccagaccctgtccctgacctgcacattcagcggcttttccctgaacacatacgatatgggagtgggatggatcagacagccacctggcaagggcctggagtggatcggcaacatctggtgggacgatgacaagtactataatccctctctgaagagcagagtgaccatctctaaggatacaagcaacaatcaggcctccctgaagctgagctccgtgaccgccgccgacacagccgtgtactattgcgccaggatcgagacagtgcgggtgagccggaagggattcgcacactggggacagggcaccctggtgacagtgtctagcgctagcaccaagggacctagcgtgttcccagaggccccttcctctaagtccacctctggaggaacagccgccctgggctgtctggtgaccgactatttccctgagccagtgacagtgtcctggaactctggggccctgaccagcggagtgcacacatttcctgccgtgctggagagctccggcctgtacagcctgtctagcgtggtgaccgtgccatcctctagcctgggcacccagacatatatctgcaacgtgaatcacaagccaagcaatacaaaggtcgacaagagagtggagcccaagtcctgtgacaagacccacacatgcccaccctgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 201 - protein, 475 aa, Artificial Sequence
  amino acid sequence of h2B1 Fab HC_2
MRPTWAWWLFLVLLLALWAPARGQVQLVESGGGLVKPSQTLSLTCTFSGFSLNTYDMGVGWIRQPPGKGLEWIGNIWWDDDKYYNPSLKSRVTISKDTSNNQASLKLSSVTAADTAVYYCARIETVRVSRKGFAHWGQGTLVTVSSASTKGPSVFPEAPSSKSTSGGTAALGCLVTDYFPEPVTVSWNSGALTSGVHTFPAVLESSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 202 - DNA, 711 bp, Artificial Sequence
  nucleotide sequence of h2B1 Fab LC_1
atgcggcccacctgggcatggtggctgttcctggtgctgctgctggccctgtgggcccccgcccggggggccaccagaatgacacagtctccaagctccttttccgcctctacaggcgacagagtgaccatcacatgcaaggccagccagtccgtgggcatcaacgtggattggtaccagcagaagcctggcaagtcccccaagctgctgatctatggggccagcaataggcacaccggcgtgccttctcgcttctctggcagcggcttcggcagggactttaccctgacaatctctagcctgcagagcgaggatttcgccacctactattgtctgcagcacggctccatcccccctacctttggcggaggcacaaaggtggagatcaagagaacagtggcggcgcccagtgtcttcatttttccccctagcgacgaagagctgaagtctgggacagccagtgtggactgttggctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 203 - protein, 237 aa, Artificial Sequence
  amino acid sequence of h2B1 Fab LC_1
MRPTWAWWLFLVLLLALWAPARGATRMTQSPSSFSASTGDRVTITCKASQSVGINVDWYQQKPGKSPKLLIYGASNRHTGVPSRFSGSGFGRDFTLTISSLQSEDFATYYCLQHGSIPPTFGGGTKVEIKRTVAAPSVFIFPPSDEELKSGTASVDCWLNNFYPREAKVQWKVDNALQSGNSQESVTEQDSKDSTYSLSSTLTLSKADYEKHKVYACEVTHQGLSSPVTKSFNRGEC

SEQ ID NO: 204 - DNA, 711 bp, Artificial Sequence
  nucleotide sequence of h2B1 Fab LC_2
atgcggcccacctgggcatggtggctgttcctggtgctgctgctggccctgtgggcccccgcccggggggccaccagaatgacacagtctccaagctccttttccgcctctacaggcgacagagtgaccatcacatgcaaggccagccagtccgtgggcatcaacgtggattggtaccagcagaagcctggcaagtcccccaagctgctgatctatggggccagcaataggcacaccggcgtgccttctcgcttctctggcagcggcttcggcagggactttaccctgacaatctctagcctgcagagcgaggatttcgccacctactattgtctgcagcacggctccatcccccctacctttggcggaggcacaaaggtggagatcaagagaacagtggcggcgcccagtgtcttcatttttccccctagcgacgaacagctgaagtctgggacagccagagtgggctgttggctgaacaacttctaccctagagaggctaaagtgcagtggaaggtcgataacgcactgcagtccggaaattctcaggagagtgtgactgaacaggactcaaaagatagcacctattccctgagaagcacactgactctgagcaaggccgactacgagaagcataaagtgtatgcttgtgaagtcacccaccaggggctgagttcaccagtcacaaaatcattcaacagaggggagtgc

SEQ ID NO: 205 - protein, 237 aa, Artificial Sequence
  amino acid sequence of h2B1 Fab LC_2
MRPTWAWWLFLVLLLALWAPARGATRMTQSPSSFSASTGDRVTITCKASQSVGINVDWYQQKPGKSPKLLIYGASNRHTGVPSRFSGSGFGRDFTLTISSLQSEDFATYYCLQHGSIPPTFGGGTKVEIKRTVAAPSVFIFPPSDEQLKSGTARVGCWLNNFYPREAKVQWKVDNALQSGNSQESVTEQDSKDSTYSLRSTLTLSKADYEKHKVYACEVTHQGLSSPVTKSFNRGEC

SEQ ID NO: 206 - DNA, 1413 bp, Artificial Sequence
  nucleotide sequence of C3E-7034 Fab HC
atgcgccccacctgggcatggtggctgttcctggtgctgctgctggccctgtgggcccctgcccggggcggcgaggtgcagctggtggagagcggcggcggcctggtgcagcccggcggctctctgcggctgagctgcgccgcctccggcgtgacattcaactactatggcatgtcctggatcagacaggccccaggcaagggcctggagtgggtggcctctatcaccaatagcggcggcaggatctactatcccgactccgtgaagggccggtttacaatctctagagagaacacccagaagacactgtacctgcagatgaacagcctgcgggccgaggataccgccgtgtactattgtacactggacggcagagacggatgggtggcatattggggacagggcaccctggtgacagtgagctccgctagcactaaaggcccaagcgtgtttcctctggctccaagctcaaaaagcacttccggggggaccgcatggctgggatgtgatgtgaccgactacttccccgagcctgtgaccgtgtcctggaactctggggccctgaccagcggagtgcacacatttccagccgtgctgcagagctccggcctgtatagcctgtctagcgtggtgacagtgccctccagtagcctgggcactcagacttatatctgtaatgtcaaccacaaaccatcaaacaccaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 207 - protein, 471 aa, Artificial Sequence
  amino acid sequence of C3E-7034 Fab HC
MRPTWAWWLFLVLLLALWAPARGGEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSASTKGPSVFPLAPSSKSTSGGTAWLGCDVTDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 208 - DNA, 714 bp, Artificial Sequence
  nucleotide sequence of C3E-7034 Fab LC
atgcggcctacctgggcctggtggctgttcctggtgctgctgctggccctgtgggccccagccaggggcaactttatgctgacccagccccacagcgtgtccgagtctcctggcaagaccgtgacaatctcctgcaagcggaacacaggcaatatcggctctaactacgtgaattggtatcagcagcacgagggcagctccccaaccacaatcatctaccgggacgataagcggcccgacggcgtgtctgataggttcagcggctccatcgaccgctctagcaagtctgccagcctgaccatcagcaatctgaagacagaggacgaggccgattacttttgtcagtcctattcctctggcttcatctttggaggaggaaccaagctgacagtgctgggccagcccaaggcggcgcccagcgtgactctgtttccacccagctccgagcagctgcaggccaataaggctagactggtctgtctgatttccgacttctaccccggggctgtgacagtcgcatggaaggccgattctagtcctgtgaaagcaggagtcgagaccacaactccatcaaagcagagcaacaacaagtacgcagcctcaagctatctgtctctgacacctgaacagtggaaaagccaccggtcttatagttgtcaggtgactcacgagggctcaacagtggaaaagacagtcgcacccgcagaatgctca

SEQ ID NO: 209 - protein, 238 aa, Artificial Sequence
  amino acid sequence of C3E-7034 Fab LC
MRPTWAWWLFLVLLLALWAPARGNFMLTQPHSVSESPGKTVTISCKRNTGNIGSNYVNWYQQHEGSSPTTIIYRDDKRPDGVSDRFSGSIDRSSKSASLTISNLKTEDEADYFCQSYSSGFIFGGGTKLTVLGQPKAAPSVTLFPPSSEQLQANKARLVCLISDFYPGAVTVAWKADSSPVKAGVETTTPSKQSNNKYAASSYLSLTPEQWKSHRSYSCQVTHEGSTVEKTVAPAECS

SEQ ID NO: 210 - DNA, 1413 bp, Artificial Sequence
  nucleotide sequence of C3E-7036 Fab HC
atgcgccccacctgggcatggtggctgttcctggtgctgctgctggccctgtgggcccctgcccggggcggcgaggtgcagctggtggagagcggcggcggcctggtgcagcccggcggctctctgcggctgagctgcgccgcctccggcgtgacattcaactactatggcatgtcctggatcagacaggccccaggcaagggcctggagtgggtggcctctatcaccaatagcggcggcaggatctactatcccgactccgtgaagggccggtttacaatctctagagagaacacccagaagacactgtacctgcagatgaacagcctgcgggccgaggataccgccgtgtactattgtacactggacggcagagacggatgggtggcatattggggacagggcaccctggtgacagtgagctccgctagcactaaaggaccaagcgtgtttccactggctccatcatccaaaagcacaagcggcgggactgcctggctgggctgtaaggtgaaggactacttccccgagcctgtgaccgtgtcctggaactctggggccctgaccagcggagtgcacacatttccagccgtgctgcagagctccggcctgtatagcctgtctagcgtggtgacagtgccctcctcaagcctgggcactcagacctatatttgtaatgtcaaccataaaccttccaacactaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 211 - protein, 471 aa, Artificial Sequence
  amino acid sequence of C3E-7036 Fab HC
MRPTWAWWLFLVLLLALWAPARGGEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSASTKGPSVFPLAPSSKSTSGGTAWLGCKVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 212 - DNA, 708 bp, Artificial Sequence
  nucleotide sequence of C3E-7036 Fab LC
atgcggcccacctgggcatggtggctgttcctggtgctgctgctggccctgtgggcccctgccaggggcaactttatgctgacccagcctagctccgtgagcggagtgccaggacagagggtgacaatctcctgcaccggcaacacaggcaatatcggctctaactacgtgaattggtatcagcagctgcccggcacagcccctaagctgctgatctaccgggacgataagcggcccagcggagtgccagacaggttctccggctctaagagcggcacctccgcctctctggccatcacaggctttcaggccgaggacgaggccgattactattgtcagtcctattctagcggcttcatctttggaggaggaaccaagctgacagtgctgggccagcccaaggcggcgcccagtgtcacactgtttccccctagctccgaggaactgcaggctaacaccgcaacactggactgtctgatcagcgacttctaccctggagctgtgactgtcgcctggaaggctgattctagtccagtgaaagcaggcgtcgagaccacaactccctctaagcagagtaacaacaagtacgcagcctcaagctatctgtcactgaccccagaacagtggaagagccaccggagctattcctgccaggtcactcacgaaggctccactgtcgagaaaaccgtcgctcccaccgaatgttca

SEQ ID NO: 213 - protein, 236 aa, Artificial Sequence
  amino acid sequence of C3E-7036 Fab LC
MRPTWAWWLFLVLLLALWAPARGNFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVLGQPKAAPSVTLFPPSSEELQANTATLDCLISDFYPGAVTVAWKADSSPVKAGVETTTPSKQSNNKYAASSYLSLTPEQWKSHRSYSCQVTHEGSTVEKTVAPTECS

SEQ ID NO: 214 - DNA, 1425 bp, Artificial Sequence
  nucleotide sequence of h2B1 Fab HC_3
atgaggcctacctgggcctggtggctgttcctggtgctgctgctggccctgtgggcccccgcccggggccaggtgcagctggtggagtccggcggcggcctggtgaagcccagccagaccctgtccctgacctgcacattcagcggcttttccctgaacacatacgatatgggagtgggatggatcagacagccacctggcaagggcctggagtggatcggcaacatctggtgggacgatgacaagtactataatccctctctgaagagcagagtgaccatctctaaggatacaagcaacaatcaggcctccctgaagctgagctccgtgaccgccgccgacacagccgtgtactattgcgccaggatcgagacagtgcgggtgagccggaagggattcgcacactggggacagggcaccctggtgacagtgtctagcgctagcacaaagggacctagcgtgttcccactggccccttcctctaagtccacctctggaggaacagccgccctgggctgtctggtgaaggactatttccctgagccagtgaccgtgtcctggaactctggggccctgaccagcggagtgcacacatttcctgccgtgctgcagagctccggcctgtacagcctgtctagcgtggtgaccgtgccatcctctagcctgggcacccagacatatatctgcaacgtgaatcacaagccaagcaatacaaaggtcgacaagagagtggagcccaagtcctgtgacaagacccacacatgcccaccctgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 215 - protein, 475 aa, Artificial Sequence
  amino acid sequence of h2B1 Fab HC_3
MRPTWAWWLFLVLLLALWAPARGQVQLVESGGGLVKPSQTLSLTCTFSGFSLNTYDMGVGWIRQPPGKGLEWIGNIWWDDDKYYNPSLKSRVTISKDTSNNQASLKLSSVTAADTAVYYCARIETVRVSRKGFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 216 - DNA, 711 bp, Artificial Sequence
  nucleotide_sequence_of_h2B1_Fab_LC_3
atgcggcccacctgggcatggtggctgttcctggtgctgctgctggccctgtgggcccccgcccggggggccaccagaatgacacagtctccaagctccttttccgcctctacaggcgacagagtgaccatcacatgcaaggccagccagtccgtgggcatcaacgtggattggtaccagcagaagcctggcaagtcccccaagctgctgatctatggggccagcaataggcacaccggcgtgccttctcgcttctctggcagcggcttcggcagggactttaccctgacaatctctagcctgcagagcgaggatttcgccacctactattgtctgcagcacggctccatcccccctacctttggcggaggcacaaaggtggagatcaagagaacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 217 - protein, 237 aa, Artificial Sequence
  amino acid sequence of h2B1 Fab LC_3
MRPTWAWWLFLVLLLALWAPARGATRMTQSPSSFSASTGDRVTITCKASQSVGINVDWYQQKPGKSPKLLIYGASNRHTGVPSRFSGSGFGRDFTLTISSLQSEDFATYYCLQHGSIPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - DNA, 1497 bp, Artificial Sequence
  nucleotide sequence of C3E-7034 scFv Fc
atgcgccctacctgggcctggtggctgttcctggtgctgctgctggccctgtgggccccagcccggggcggcgaggtgcagctggtggagtctggcggcggcctggtgcagcccggcggcagcctgcggctgtcctgcgccgcctctggcgtgaccttcaactactatggcatgagctggatcagacaggcccccggcaagggcctggagtgggtggccagcatcaccaattccggcggcaggatctactatcctgactctgtgaagggcaggtttacaatcagccgcgagaacacccagaagacactgtacctgcagatgaacagcctgcgggccgaggataccgccgtgtactattgcacactggacggcagagacggatgggtggcatattggggacagggcaccctggtgacagtgagctccggcggcggcggctctggaggaggaggcagcggcggaggaggctccaactttatgctgacccagccccactctgtgagcgagtcccctggcaagaccgtgacaatctcctgtaagagaaacacaggcaatatcggctctaactacgtgaattggtatcagcagcacgagggctctagcccaaccacaatcatctaccgggacgataagcggcccgacggcgtgagcgatcggttctctggcagcatcgacagatcctctaagtccgcctctctgaccatcagcaatctgaagacagaggacgaggccgattacttttgtcagtcctatagctccggcttcatctttggcggcggcaccaagctgacagtgctggcggccgagcctaaaagtagcgataaaacccatacctgccccccctgcccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 219 - protein, 499 aa, Artificial Sequence
  amino acid sequence of C3E-7034 scFv Fc
MRPTWAWWLFLVLLLALWAPARGGEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRDDKRPDGVSDRFSGSIDRSSKSASLTISNLKTEDEADYFCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 220 - DNA, 1491 bp, Artificial Sequence
  nucleotide sequence of C3E-7036 scFv Fc
atgcggcccacctgggcatggtggctgttcctggtgctgctgctggccctgtgggcccctgccaggggcggcgaggtgcagctggtggagagcggcggcggcctggtgcagcctggaggctctctgaggctgagctgcgcagcctccggcgtgacattcaactactatggcatgtcctggatcagacaggccccaggcaagggcctggagtgggtggcctctatcaccaatagcggcggcagaatctactatcccgactccgtgaagggcaggtttacaatcagccgggagaacacccagaagacactgtacctgcagatgaatagcctgcgggccgaggataccgccgtgtactattgcacactggacggcagagatggatgggtggcatattggggacagggcaccctggtgacagtgagctccggaggaggaggctccggcggcggaggctctggcggcggcggcagcaactttatgctgacccagccttctagcgtgagcggagtgccaggacagagggtgacaatctcctgtaccggcaacacaggcaatatcggctctaactacgtgaattggtatcagcagctgccaggaaccgcccctaagctgctgatctaccgggacgataagcggccctctggagtgccagaccggttcagcggctccaagtctggcaccagcgcctccctggccatcacaggatttcaggccgaggacgaggccgattactattgtcagtcctattcctctggcttcatctttggcggcggcaccaagctgacagtgctggcggccgagcctaaaagtagcgataaaacccatacctgccccccctgcccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 221 - protein, 497 aa, Artificial Sequence
  amino acid sequence of C3E-7036 scFv Fc
MRPTWAWWLFLVLLLALWAPARGGEVQLVESGGGLVQPGGSLRLSCAASGVTFNYYGMSWIRQAPGKGLEWVASITNSGGRIYYPDSVKGRFTISRENTQKTLYLQMNSLRAEDTAVYYCTLDGRDGWVAYWGQGTLVTVSSGGGGSGGGGSGGGGSNFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 222 - DNA, 1512 bp, Artificial Sequence
  nucleotide sequence of h2B1 scFv Fc
atgcggcctacctgggcctggtggctgttcctggtgctgctgctggccctgtgggcccccgcccggggggccacccgcatgacacagtcccctagctccttttctgccagcacaggcgatagagtgaccatcacatgcaaggcctcccagtctgtgggcatcaacgtggactggtaccagcagaagcctggcaagagcccaaagctgctgatctatggggccagcaataggcacaccggagtgccatctagattcagcggctccggcttcggcagagacttcaccctgacaatctctagcctgcagagcgaggacttcgccacatactattgcctgcagcacggctccatcccccctacctttggcggaggcacaaaggtggagatcaagggaggaggaggctccggcggaggaggctctggcggcggcggcagcggcggcggccaggtgcagctggtggagtccggaggaggcctggtgaagccaagccagaccctgtccctgacctgtacattctctggctttagcctgaacacatacgatatgggagtgggatggatcaggcagccaccaggcaagggcctggagtggatcggcaacatctggtgggacgatgacaagtactataatccctccctgaagtctcgcgtgaccatctctaaggatacaagcaacaatcaggcctctctgaagctgtcctctgtgaccgccgccgacacagccgtgtactattgtgcaaggatcgagacagtgcgggtgagccggaagggatttgcacactggggacagggcaccctggtgacagtgagctctgcggccgagcctaaaagtagcgataaaacccatacctgccccccctgcccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 223 - protein, 504 aa, Artificial Sequence
  amino_acid_sequence_of_h2B1_scFv_Fc
MRPTWAWWLFLVLLLALWAPARGATRMTQSPSSFSASTGDRVTITCKASQSVGINVDWYQQKPGKSPKLLIYGASNRHTGVPSRFSGSGFGRDFTLTISSLQSEDFATYYCLQHGSIPPTFGGGTKVEIKGGGGSGGGGSGGGGSGGGQVQLVESGGGLVKPSQTLSLTCTFSGFSLNTYDMGVGWIRQPPGKGLEWIGNIWWDDDKYYNPSLKSRVTISKDTSNNQASLKLSSVTAADTAVYYCARIETVRVSRKGFAHWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 224 - DNA, 1491 bp, Artificial Sequence
  nucleotide sequence of C3E-8015
atgcggcccacctgggcatggtggctgttcctggtgctgctgctggccctgtgggcccctgccaggggcggagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaggtccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctcactcagccgtcctctgtttctggcgtacctggccaacgggtgaccattagctgtacgggtaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgttggcggccgagcctaaaagtagcgataaaacccatacctgccccccctgcccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 225 - protein, 497 aa, Artificial Sequence
  amino acid sequence of C3E-8015
MRPTWAWWLFLVLLLALWAPARGGEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 226 - DNA, 1497 bp, Artificial Sequence
  nucleotide sequence of C3E-8017
atgcgccctacctgggcctggtggctgttcctggtgctgctgctggccctgtgggccccagcccggggcggagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaggtccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctgacccagccccacagtgtgtcagagagccctggcaagactgtcaccatctcttgtaaaaggaacaccggaaatattggcagtaactacgtgaattggtatcagcagcatgaagggtctagtccaaccacaatcatctaccggaacgataagagacccgacggggtgtccgatcgattctccggatctatcgaccggtcaagcaagagtgcttcactgaccattagcaatctgaaaacagaggacgaagcagattacttttgccagtcctattcctctggcttcatctttggaggcgggactaaactgaccgtgctggcggccgagcctaaaagtagcgataaaacccatacctgccccccctgcccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 227 - protein, 499 aa, Artificial Sequence
  amino acid sequence of C3E-8017
MRPTWAWWLFLVLLLALWAPARGGEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 228 - DNA, 1497 bp, Artificial Sequence
  nucleotide sequence of C3E-8018
atgcgccctacctgggcctggtggctgttcctggtgctgctgctggccctgtgggccccagcccggggcggagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcacttcttccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctgacccagccccacagtgtgtcagagagccctggcaagactgtcaccatctcttgtaaaaggaacaccggaaatattggcagtaactacgtgaattggtatcagcagcatgaagggtctagtccaaccacaatcatctaccggaacgataagagacccgacggggtgtccgatcgattctccggatctatcgaccggtcaagcaagagtgcttcactgaccattagcaatctgaaaacagaggacgaagcagattacttttgccagtcctattcctctggcttcatctttggaggcgggactaaactgaccgtgctggcggccgagcctaaaagtagcgataaaacccatacctgccccccctgcccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 229 - protein, 499 aa, Artificial Sequence
  amino acid sequence of C3E-8018
MRPTWAWWLFLVLLLALWAPARGGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 230 - DNA, 1494 bp, Artificial Sequence
  nucleotide sequence of C3E-8025
atgcggcccacctgggcatggtggctgttcctggtgctgctgctggccctgtgggcccctgccaggggcggagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaggtccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctcactcagccgtcctctgtttctggcgtacctggccaacgggtgaccattagctgtacgggtaataccgggaatatcgggtctaactacgtgaactggtatcagcagcttccagggacagctcccaagttgctgatctatcgcgacgacaaaagaccctcaggggtccctgaccgatttagtggcagcaaaagcggtacttccgcttccctggcgataaccggctttcaggccgaagatgaggcagactactattgccagtcatattccagcggcttcatcttcggaggcggaactaagctgacagtgttggcggccgagcctaaaagtagcgataaaacccatacctgccccccctgcccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggcaag

SEQ ID NO: 231 - protein, 498 aa, Artificial Sequence
  amino acid sequence of C3E-8025
MRPTWAWWLFLVLLLALWAPARGGEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 232 - DNA, 1500 bp, Artificial Sequence
  nucleotide sequence of C3E-8027
atgcgccctacctgggcctggtggctgttcctggtgctgctgctggccctgtgggccccagcccggggcggagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcactaggtccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctgacccagccccacagtgtgtcagagagccctggcaagactgtcaccatctcttgtaaaaggaacaccggaaatattggcagtaactacgtgaattggtatcagcagcatgaagggtctagtccaaccacaatcatctaccggaacgataagagacccgacggggtgtccgatcgattctccggatctatcgaccggtcaagcaagagtgcttcactgaccattagcaatctgaaaacagaggacgaagcagattacttttgccagtcctattcctctggcttcatctttggaggcgggactaaactgaccgtgctggcggccgagcctaaaagtagcgataaaacccatacctgccccccctgcccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggcaag

SEQ ID NO: 233 - protein, 500 aa, Artificial Sequence
  amino acid sequence of C3E-8027
MRPTWAWWLFLVLLLALWAPARGGEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 234 - DNA, 1500 bp, Artificial Sequence
  nucleotide sequence of C3E-8028
atgcgccctacctgggcctggtggctgttcctggtgctgctgctggccctgtgggccccagcccggggcggagaagtgcagctggtggaatccggggggggcctggtgcagcctggggggagcctgagactgagttgtgccgcctctggggtgacatttaactactatggcatgtcttggatccgccaggcacctggaaagggcctggagtgggtggccagcatcacttcttccggcgggcgaatctactatcccgacagcgtcaagggcaggttcacaatttcccgcgagaacacacagaaaactctgtacctgcagatgaatagcctgagagccgaagatacagctgtgtactattgcactctggacggcagggatgggtgggtcgcctattgggggcagggaaccctggtgacagtcagctccggaggaggaggatctggcggaggaggcagtgggggaggcgggtcaaactttatgctgacccagccccacagtgtgtcagagagccctggcaagactgtcaccatctcttgtaaaaggaacaccggaaatattggcagtaactacgtgaattggtatcagcagcatgaagggtctagtccaaccacaatcatctaccggaacgataagagacccgacggggtgtccgatcgattctccggatctatcgaccggtcaagcaagagtgcttcactgaccattagcaatctgaaaacagaggacgaagcagattacttttgccagtcctattcctctggcttcatctttggaggcgggactaaactgaccgtgctggcggccgagcctaaaagtagcgataaaacccatacctgccccccctgcccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggcaag

SEQ ID NO: 235 - protein, 500 aa, Artificial Sequence
  amino acid sequence of C3E-8028
MRPTWAWWLFLVLLLALWAPARGGEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 236 - DNA, 1428 bp, Artificial Sequence
  nucleotide sequence of h2B1 Fab HC_4
atgaggcctacctgggcctggtggctgttcctggtgctgctgctggccctgtgggcccccgcccggggccaggtgcagctggtggagtccggcggcggcctggtgaagcccagccagaccctgtccctgacctgcacattcagcggcttttccctgaacacatacgatatgggagtgggatggatcagacagccacctggcaagggcctggagtggatcggcaacatctggtgggacgatgacaagtactataatccctctctgaagagcagagtgaccatctctaaggatacaagcaacaatcaggcctccctgaagctgagctccgtgaccgccgccgacacagccgtgtactattgcgccaggatcgagacagtgcgggtgagccggaagggattcgcacactggggacagggcaccctggtgacagtgtctagcgctagcacaaagggacctagcgtgttcccactggccccttcctctaagtccacctctggaggaacagccgccctgggctgtctggtgaaggactatttccctgagccagtgaccgtgtcctggaactctggggccctgaccagcggagtgcacacatttcctgccgtgctgcagagctccggcctgtacagcctgtctagcgtggtgaccgtgccatcctctagcctgggcacccagacatatatctgcaacgtgaatcacaagccaagcaatacaaaggtcgacaagagagtggagcccaagtcctgtgacaagacccacacatgcccaccctgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggcaag

SEQ ID NO: 237 - protein, 476 aa, Artificial Sequence
  nucleotide_sequence_of_h2B1_Fab_HC_4
MRPTWAWWLFLVLLLALWAPARGQVQLVESGGGLVKPSQTLSLTCTFSGFSLNTYDMGVGWIRQPPGKGLEWIGNIWWDDDKYYNPSLKSRVTISKDTSNNQASLKLSSVTAADTAVYYCARIETVRVSRKGFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 238 - protein, 8 aa, Artificial Sequence
  amino_acid_sequence_of_modified_H_CDR2
  MISC_FEATURE (2)..(2): The first Xaa is any naturally occurring amino acid
  MISC_FEATURE (3)..(3): The second Xaa is any naturally occurring amino acid
  misc_feature (4)..(4): Xaa can be any naturally occurring amino acid
ITXXGGRI

SEQ ID NO: 239 - protein, 3 aa, Artificial Sequence
  amino_acid_sequence_of_modified_L_CDR2
  MISC_FEATURE (2)..(2): Xaa is any naturally occurring amino acid
  misc_feature (5)..(5): Xaa can be any naturally occurring amino acid
RXD

SEQ ID NO: 240 - protein, 118 aa, Artificial Sequence
  amino_acid_sequence_of_VH_of_variant_of_C3E- 7034
  MISC_FEATURE (53)..(54): The first and second Xaa′s are any naturally occurring amino acid
EVQLVESGGGLVQPGGSLRLSCAASGVTFNYYGMSWIRQAPGKGLEWVASITXXGGRIYYPDSVKGRFTISRENTQKTLYLQMNSLRAEDTAVYYCTLDGRDGWVAYWGQGTLVTVSS

SEQ ID NO: 241 - protein, 109 aa, Artificial Sequence
  amino_acid_sequence_of_VL_of_variant_of_C3E- 7034
  MISC_FEATURE (52)..(52): Xaa is any naturally occurring amino acid
NFMLTQPHSVSESPGKTVTISCKRNTGNIGSNYVNWYQQHEGSSPTTIIYRXDKRPDGVSDRFSGSIDRSSKSASLTISNLKTEDEADYFCQSYSSGFIFGGGTKLTVL

SEQ ID NO: 242 - protein, 107 aa, Artificial Sequence
  amino_acid_sequence_of_VL_of_variant_of_C3E- 7035
  MISC_FEATURE (52)..(52): Xaa is any naturally occurring amino acid
QAVLTQPSSVSGVPGQRVTISCKRNTGNIGSNYVNWYQQLPGTAPKLLIYRXDKRPSGVPDRFSGSKSGTSASLAITGFQAEDEADYYCQSYSSGFIFGGGTKLTVL

SEQ ID NO: 243 - protein, 107 aa, Artificial Sequence
  amino_acid_sequence_of_VL_of_variant_of_C3E- 7036
  MISC_FEATURE (52)..(52): Xaa is any naturally occurring amino acid
NFMLTQPSSVSGVPGQRVTISCTGNTGNIGSNYVNWYQQLPGTAPKLLIYRXDKRPSGVPDRFSGSKSGTSASLAITGFQAEDEADYYCQSYSSGFIFGGGTKLTVL

SEQ ID NO: 244 - protein, 269 aa, Artificial Sequence
  amino_acid_sequence_of_C3E-7078
GEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRDDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 245 - protein, 267 aa, Artificial Sequence
  amino_acid_sequence_of_C3E-7085
GEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVLGAAAGAGGDYKDDDDKGAAAHHHHHH

SEQ ID NO: 246 - protein, 269 aa, Artificial Sequence
  amino_acid_sequence_of_C3E-7086
GEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRGDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 247 - protein, 269 aa, Artificial Sequence
  amino_acid_sequence_of_C3E-7087
GEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRQDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 248 - protein, 269 aa, Artificial Sequence
  amino_acid_sequence_of_C3E-7088
GEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 249 - protein, 269 aa, Artificial Sequence
  amino_acid_sequence_of_C3E-7089
GEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRSDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 250 - protein, 269 aa, Artificial Sequence
  amino_acid_sequence_of_C3E-7090
GEVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRADKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 251 - protein, 269 aa, Artificial Sequence
  amino_acid_sequence_of_C3E-7091
GEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRGDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 252 - protein, 269 aa, Artificial Sequence
  amino_acid_sequence_of_C3E-7092
GEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRQDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 253 - protein, 269 aa, Artificial Sequence
  amino_acid_sequence_of_C3E-7093
GEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 254 - protein, 269 aa, Artificial Sequence
  amino_acid_sequence_of_C3E-7094
GEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRSDKRPDGVSDRFSGSIDRSSKSASLTISNLKTEDEADYFCQSYSSGFIFGGGTKLTVLGAAAGAGGDYKDDDDKGAAAHHHHHH

SEQ ID NO: 255 - protein, 269 aa, Artificial Sequence
  amino_acid_sequence_of_C3E-7095
GEVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSSGGGGSGGGGSGGGGSNFMLTQPHSVSESPGKTVTISCKRNTGNIGSNYVNWYQQHEGSSPTTIIYRADKRPDGVSDRFSGSIDRSSKSASLTISNLKTEDEADYFCQSYSSGFIFGGGTKLTVLGAAAGAGGDYKDDDDKGAAAHHHHHH